(19) United States
(12) Patent Application Publication
(10) Pub. No.: US 2026/0217681 A1
(43) Pub. Date: Jul. 30, 2026

(54) HYDROXYLAMINE COMPOUNDS FOR TREATING NEURODEGENERATIVE METABOLIC DISEASES

(71) Applicant: Zevra Therapeutics, Inc., Celebration, FL (US)

(72) Inventors: Sanjib Bera, Celebration, FL (US); Guochen Chi, Celebration, FL (US); Amarraj Chakraborty, Celebration, FL (US); Sven Guenther, Coralville, IA (US); Travis Mickle, Celebration, FL (US)

(21) Appl. No.: 19/430,160

(22) Filed: Dec. 22, 2025

Related U.S. Application Data
(60) Provisional application No. 63/738,122, filed on Dec. 23, 2024.

(51) Int. Cl. C07D 401/14; C07D 401/12

(52) U.S. Cl. CPC C07D 401/14; C07D 401/12

(57) ABSTRACT

The present technology related to a genus of compounds which are prodrugs of arimoclomol. The present technology further relates to compositions comprising the same and methods of treating neurological diseases and disorders by administering said compounds.

### STATEMENT OF RELATED APPLICATIONS

This application claim priority to U.S. Patent Application No. 63/738,122, filed on Dec. 23, 2024, the entire contents of which are incorporated herein by reference.

### STATEMENT OF GOVERNMENTAL FUNDING

No Federal funds were used in developing instant technology.

### BACKGROUND OF THE INVENTION

The lysosomal storage diseases are a rare group of diseases, characterized by the accumulation of substances in the lysosomal compartment and resulting destabilization hereof, with a resulting devastating effect for affected individuals. Substances accumulate in the lysosomal compartment due to deficiencies in the enzymes involved in their catabolism.

Niemann-Pick disease and Gaucher's Disease are two lysosomal disorders characterized by build-up of fatty substances in the cells. Niemann-Pick disease is caused by the build-up of cholesterol in neurons. Gaucher disease is caused by a buildup of lipids in organs such as the spleen and liver. Neither Niemann-Pick disease or Gaucher's disease can be cured. The only treatment is to reduce the build-up of the problem-substance through oral medication and/or enzyme replacement therapy.

Currently, oral treatments for lysosomal storage disorders are accomplished by multiple doses of drugs such as arimoclomol and/or salts thereof and miglustat.

Currently, there exists a need in the art for prodrug-forms of arimoclomol, or compositions thereof, with slower absorption and/or longer half-life that result in longer release profiles that provide more consistent plasma levels of the drugs. There further exists a need in the art for forms of arimoclomol, or compositions thereof, that can provide therapeutically effective plasma levels with 1 or 2 oral dosages per day.

### SUMMARY OF THE INVENTION

The present technology provides hydroxylamine compound, which are prodrugs of arimoclomol, with improved pharmacokinetics. The present technology further provides compositions comprising hydroxylamine compounds. The present technology still further provides methods for treating neurodegenerative or metabolic diseases and/or disorders using said hydroxylamine compounds and compositions thereof.

In certain aspects, the present technology relates to a genus of compounds having a structure of Formula I:

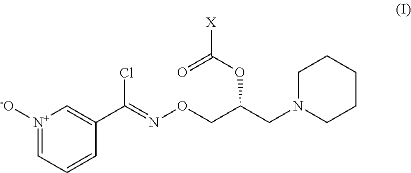

wherein X is selected from the group consisting of R, [O—R], [NR¹R²], and [NRH]
wherein O is oxygen, N is nitrogen, and
R, R¹ and R² are independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol
or a pharmaceutically acceptable salt thereof.

In a further aspect, the present technology relates to a compound of Formula I, wherein the compound of Formula I is a compound having a structure of Formula IA:

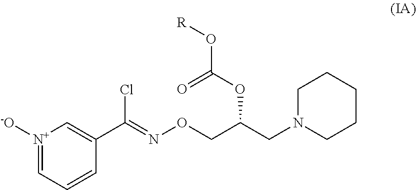

wherein R is selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a still further aspect, the present technology relates to a compound of Formula IA, wherein the compound of IA is a compound having a structure selected from:

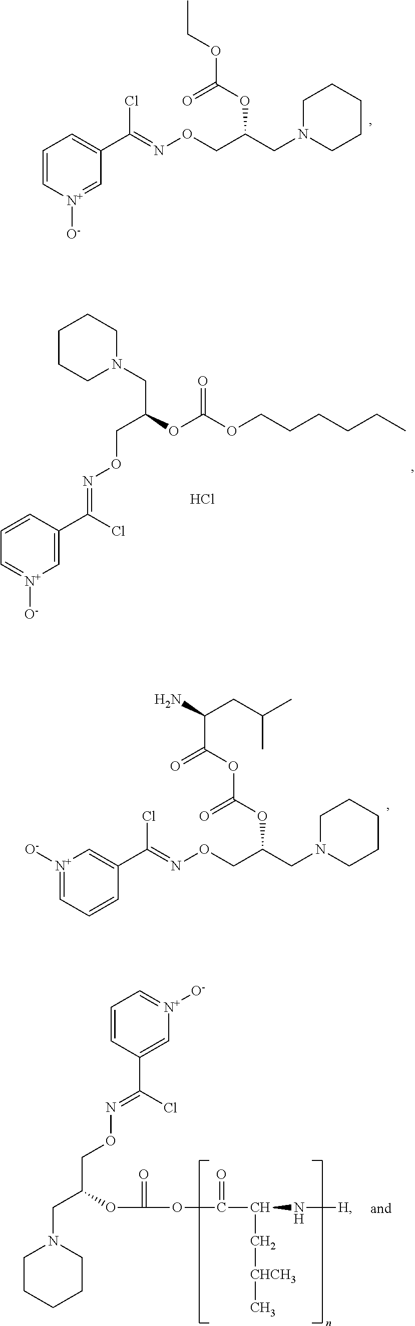

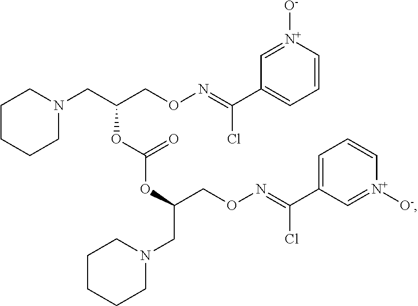

wherein n is 1-50;
or a pharmaceutically acceptable salt thereof.

In a further aspect, the present technology relates to a compound of Formula IA, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In a further aspect, the present technology relates to a compound of Formula I, wherein the compound of Formula I is a compound having a structure of Formula IB:

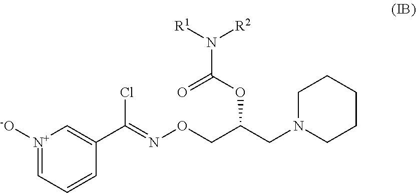

wherein R¹ and R² are independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a still further aspect, the present technology relates to a compound of Formula IB, wherein the compound of Formula IB is a compound having a structure selected from:

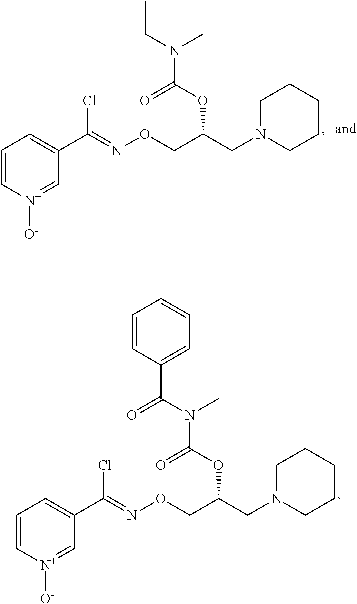 [SMILES: CCN(C)C(=O)O[C@@H](CO/N=C(\Cl)c1ccc[n+]([O-])c1)CN1CCCCC1 ; CN(C(=O)O[C@@H](CO/N=C(\Cl)c1ccc[n+]([O-])c1)CN1CCCCC1)C(=O)c1ccccc1]

or a pharmaceutically acceptable salt thereof.

In a further aspect, the present technology relates to a compound of Formula I, wherein the compound of Formula I is a compound of Formula IC:

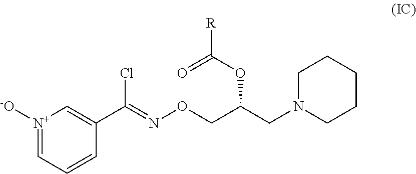

wherein R is selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a still further aspect, the present technology relates to a compound of Formula IC, wherein the compound of Formula IC is a compound having a structure selected from:

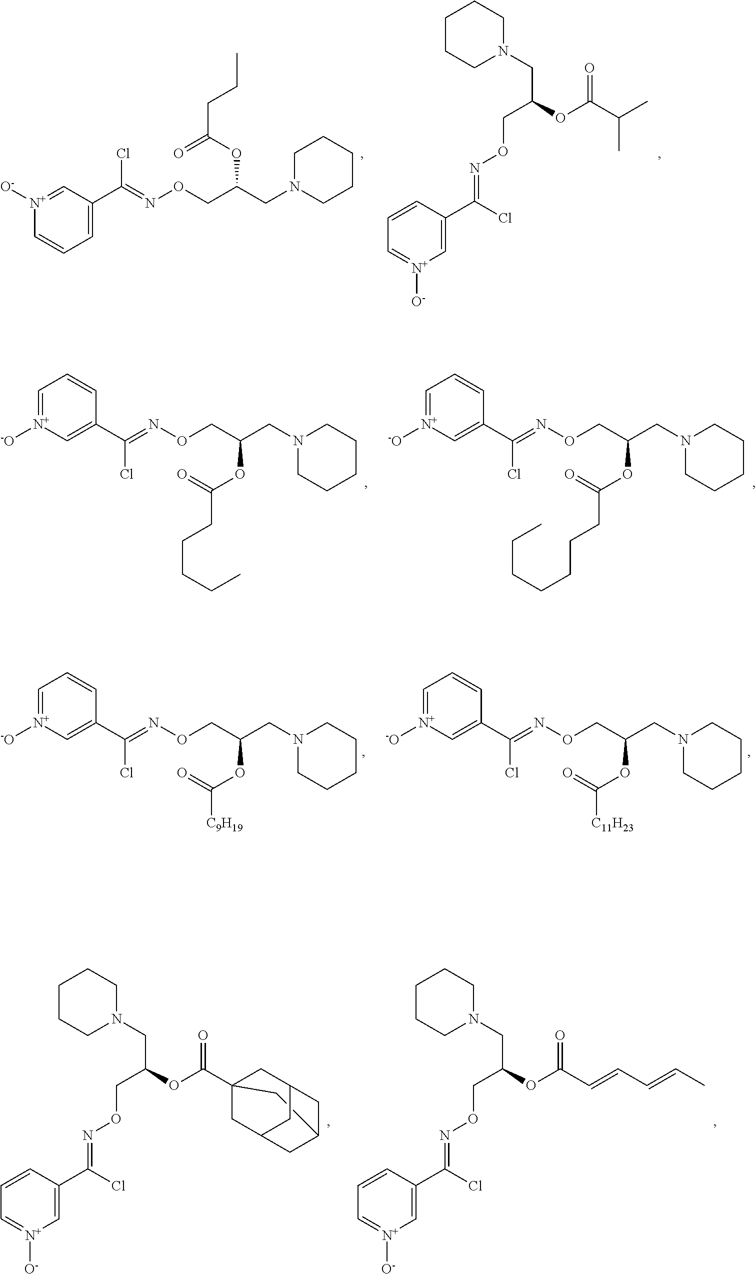

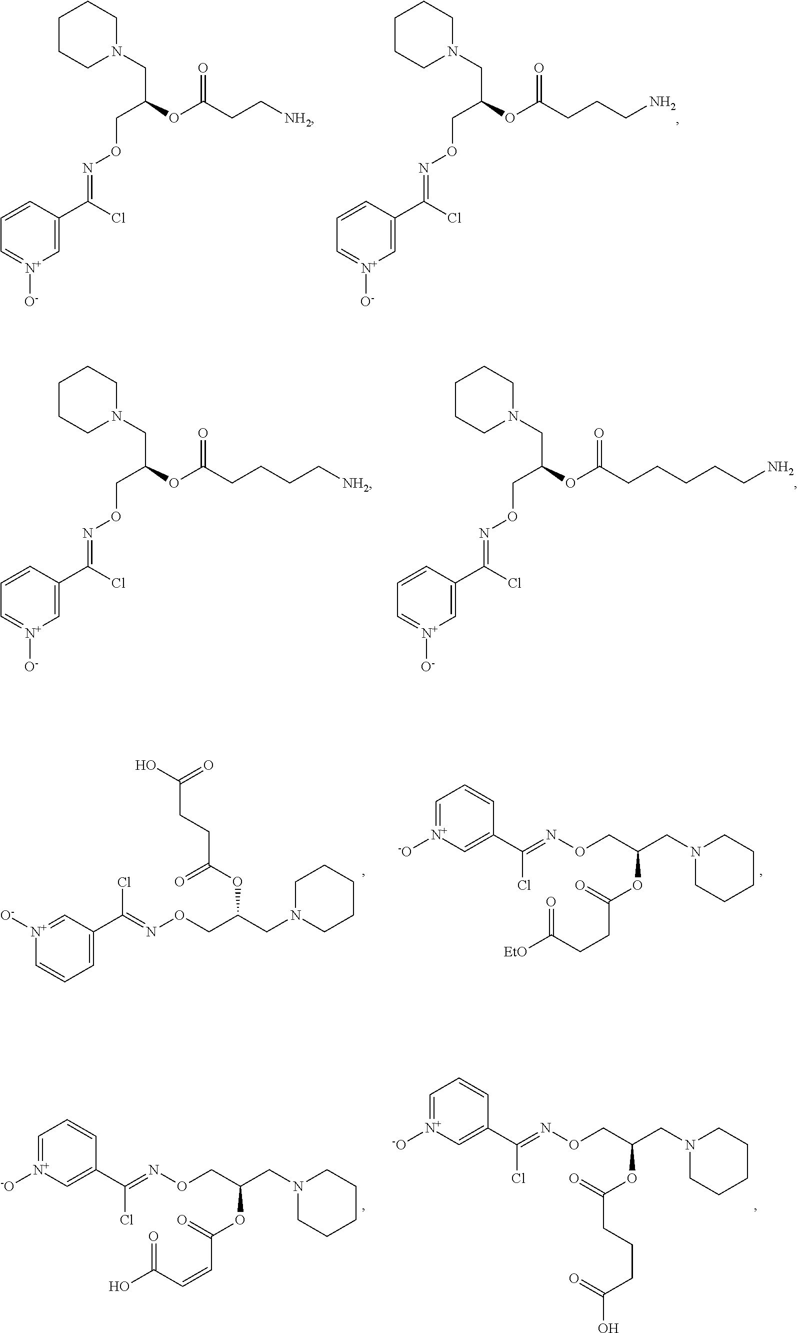

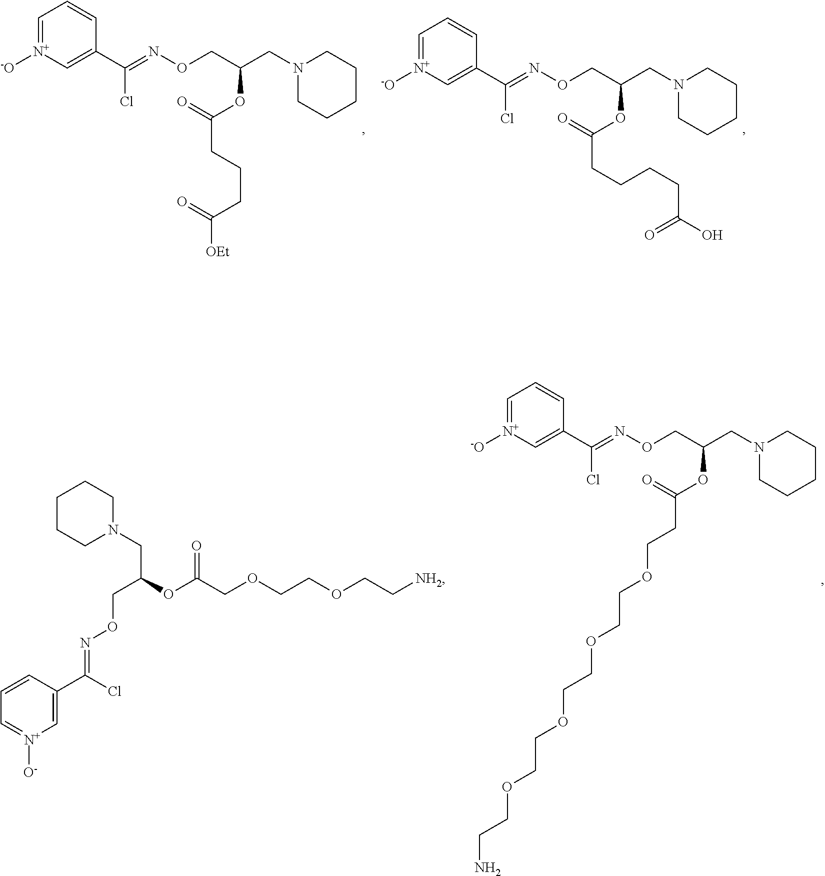

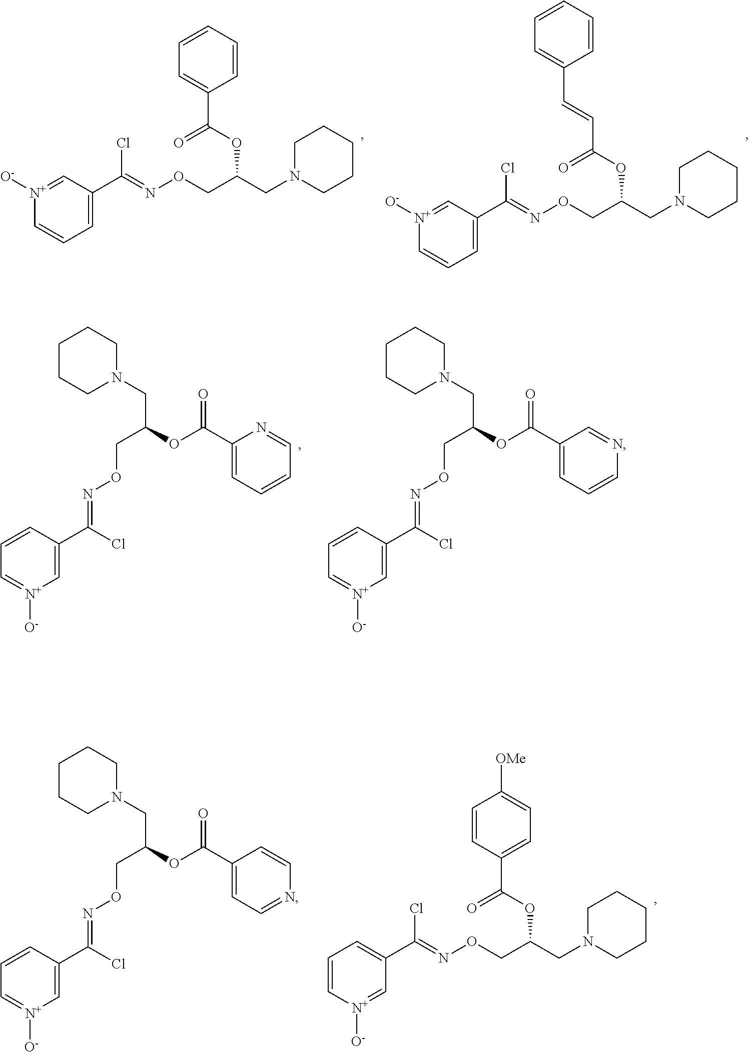

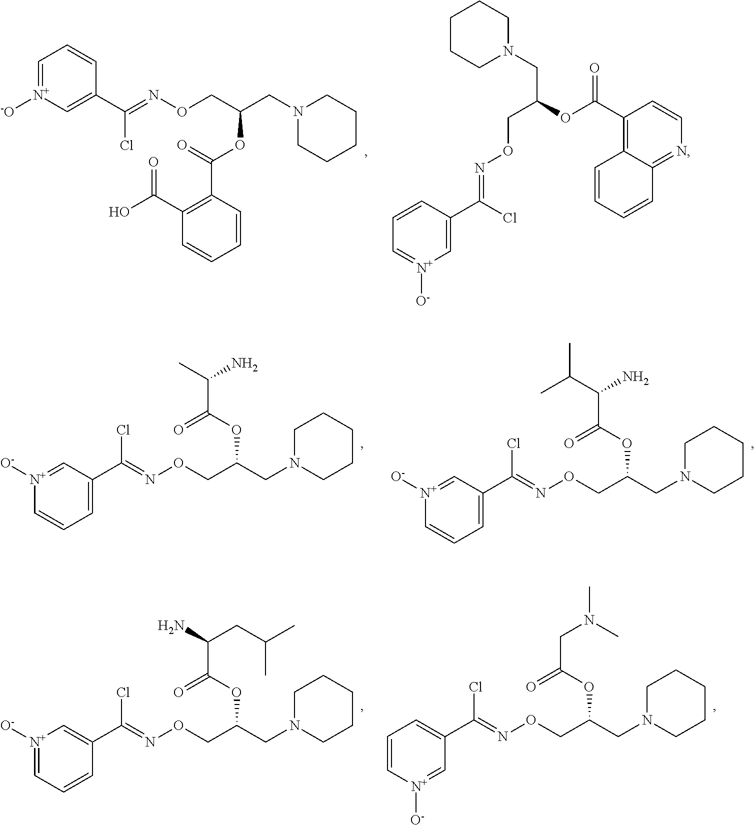

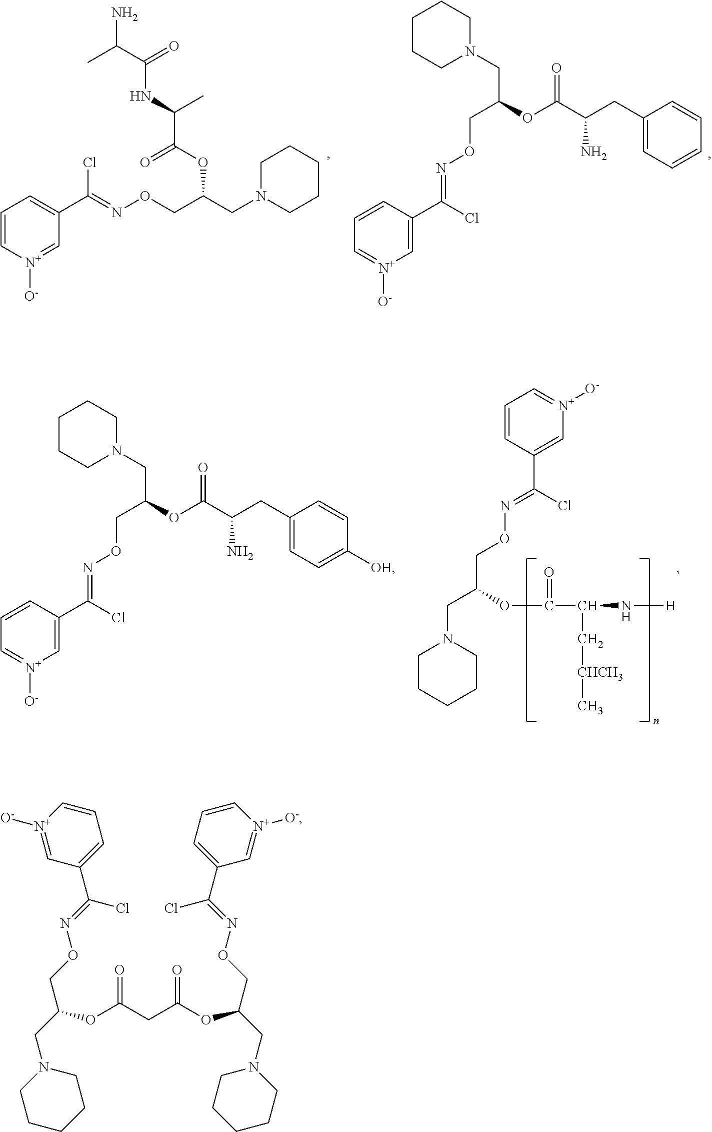

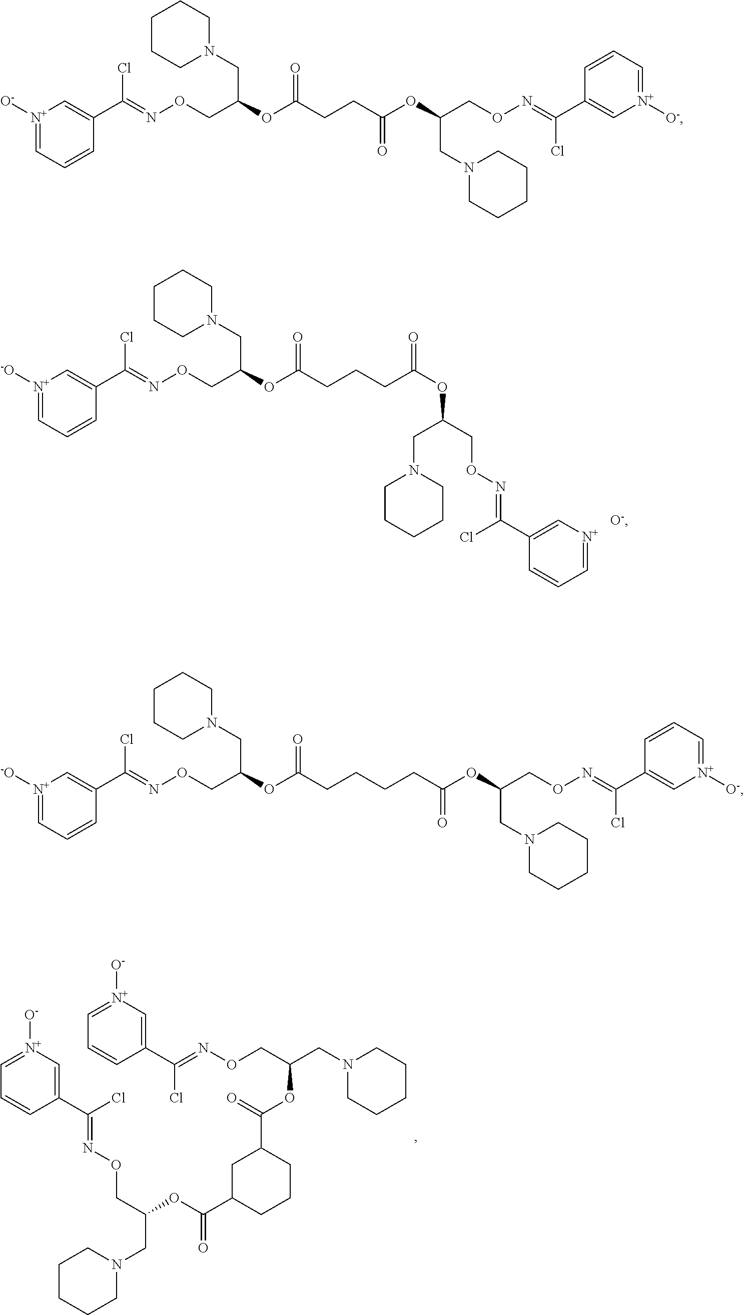

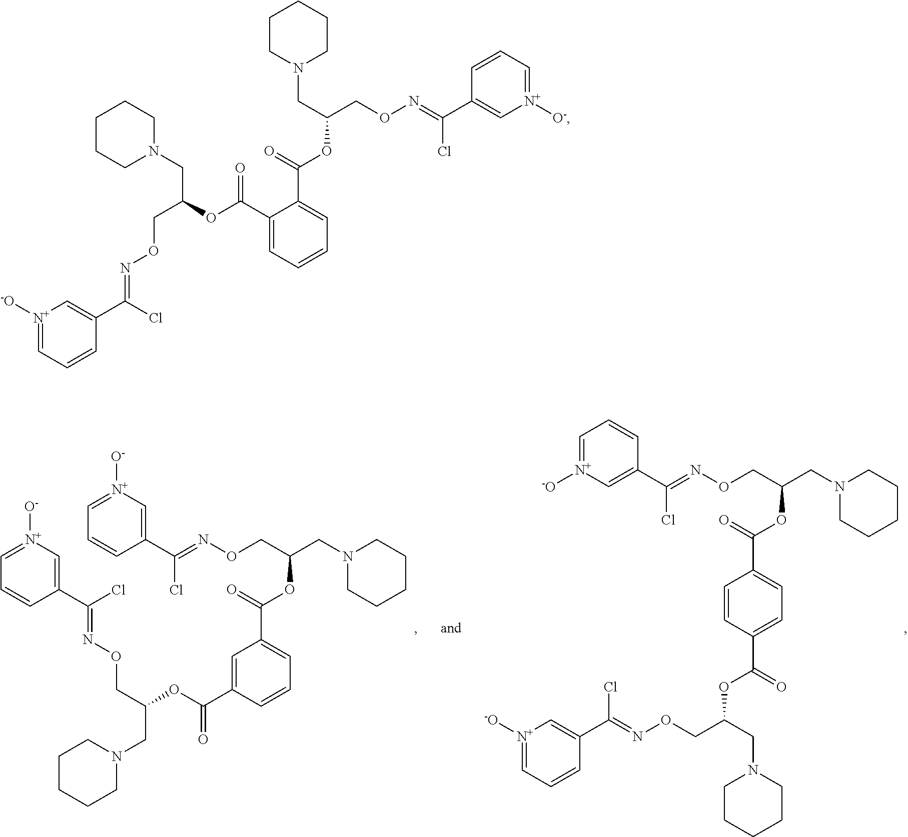

wherein n is 1 to 50;
or a pharmaceutically acceptable salt thereof.

In a further aspect, the present technology relates to a compound of Formula IC, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In a still further aspect, the present technology relates to a compound of Formula IC, wherein the compound of IC is a compound selected from:

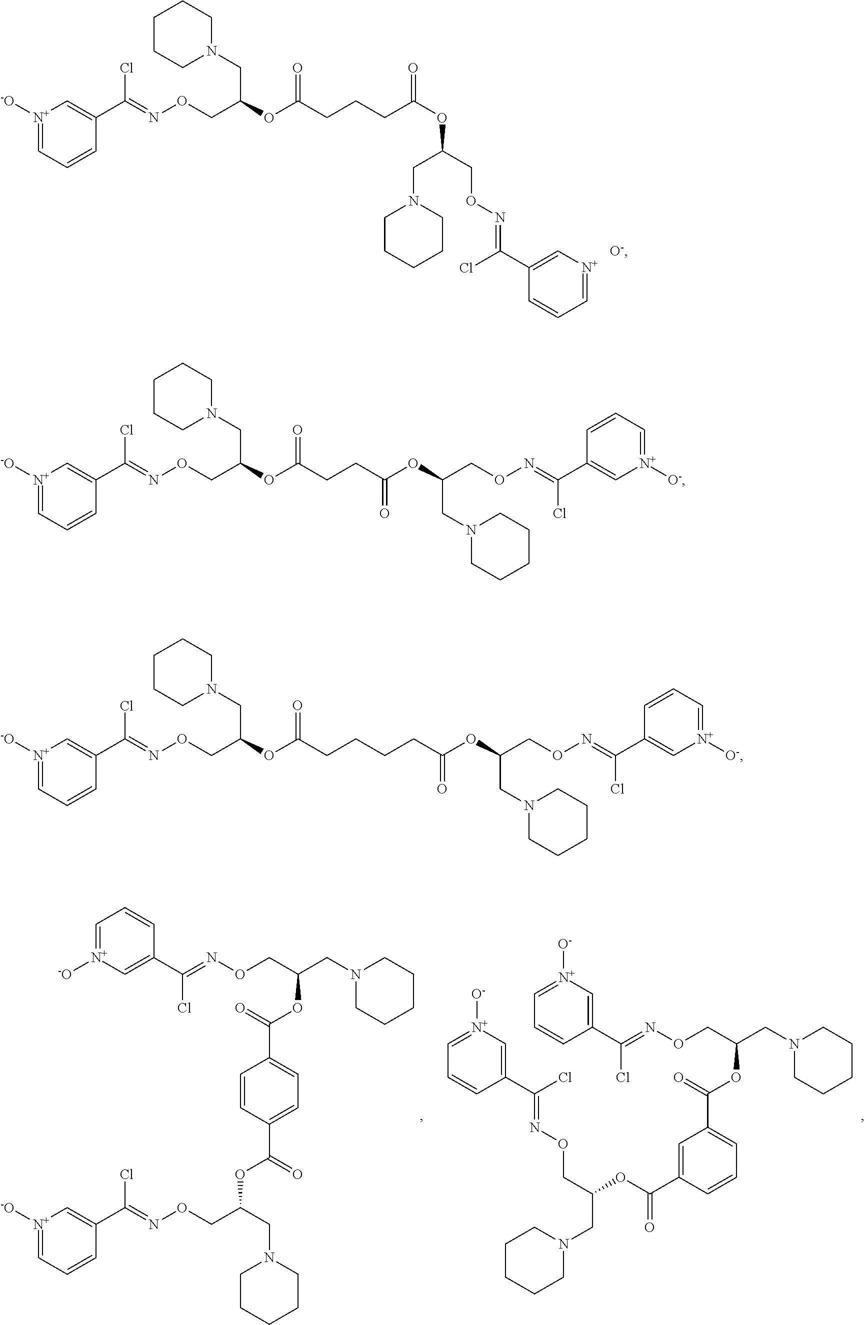

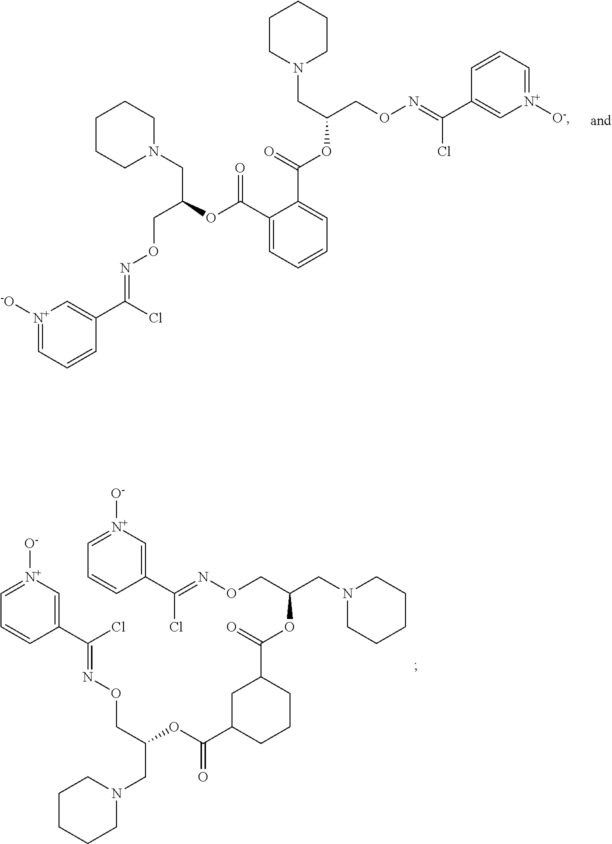

or a pharmaceutically acceptable salt thereof.

In a further aspect, the present technology relates to a compound of Formula I, wherein the compound of Formula I is a compound of Formula ID:

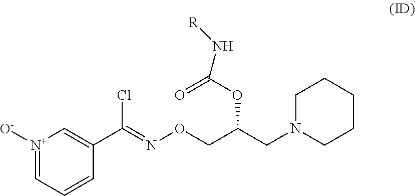

wherein R is selected from the group consisting of alkenyl, alkenyl, aminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a still further aspect, the present technology relates to a compound of Formula ID, wherein the compound of Formula ID is a compound having a structure selected from:

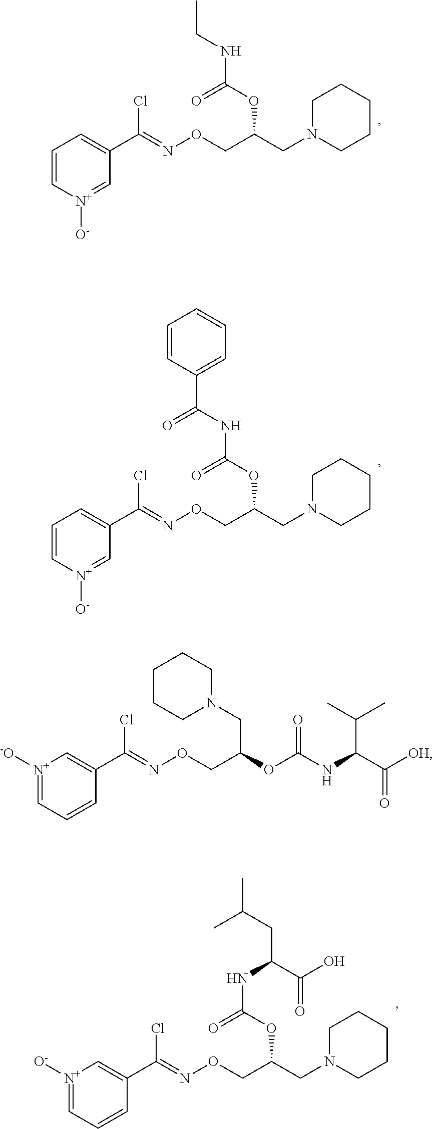

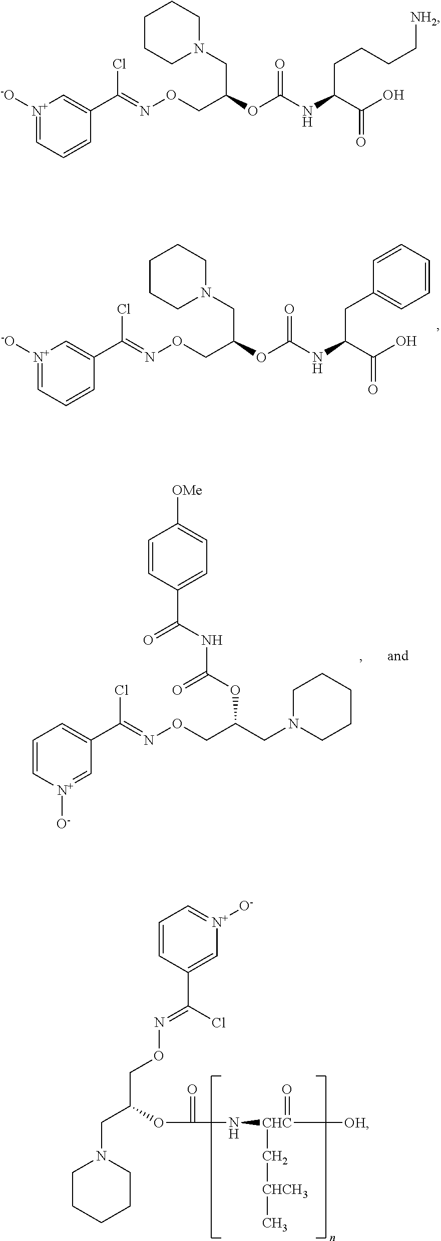

wherein n is 1-50;
or a pharmaceutically acceptable salt thereof.

In a further aspect, the present technology relates to a compound of Formula I, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In certain aspects, the present technology relates to a compound of Formula II:

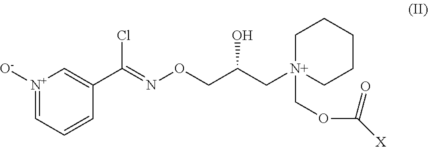

wherein X is selected from the group consisting of R, [O—R], [NR¹R²], and [NRH]
wherein O is oxygen, N is nitrogen, H is hydrogen, and
R, R¹ and R² are independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol
or a pharmaceutically acceptable salt thereof.

In a still further aspect, the present technology relates to a compound of Formula II, wherein the compound of Formula II has a structure selected from

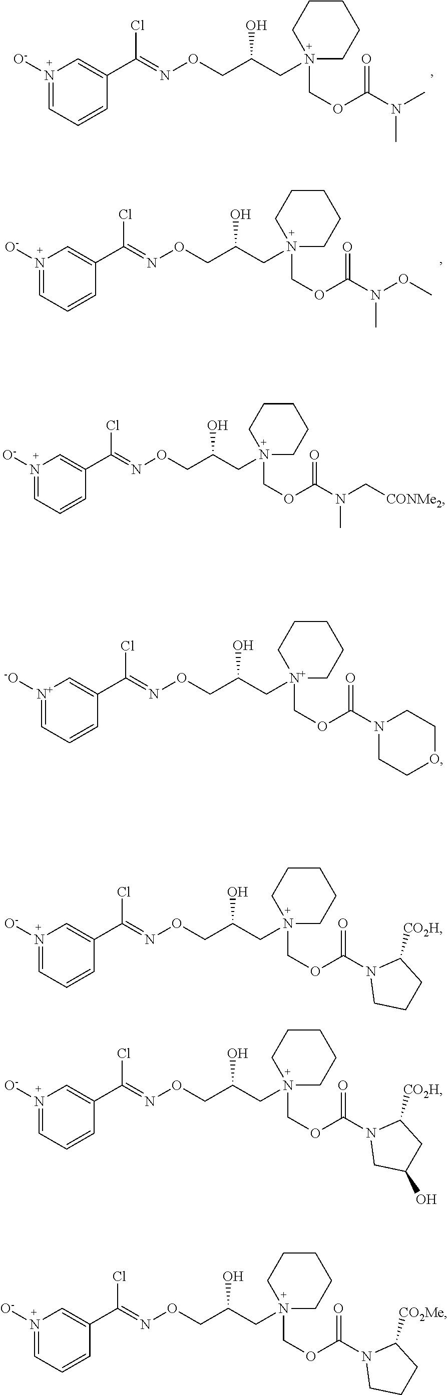

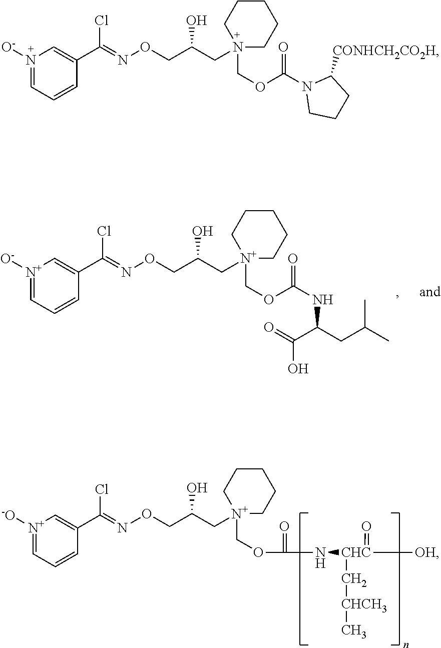

wherein n is 1-50; or a pharmaceutically acceptable salt thereof.

In a further aspect, the present technology relates to a compound of Formula II, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In certain aspects, the present technology relates to a compound of Formula III:

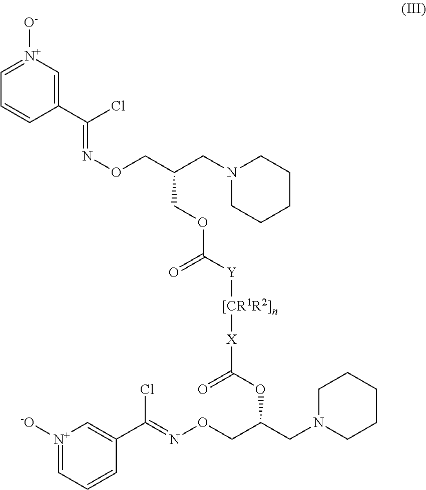

wherein X is selected from the group consisting of O, N, or absent,
Y is selected from the group consisting of O, N, or absent,
n is 1-10,
R¹ and R² are selected independently from each other and for each CR¹R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a further aspect, the present technology relates to the compound of Formula IV, wherein n is alternatively 1-3, alternatively 3-5, alternatively 5-7, alternatively 7-9, alternatively 1-5, alternatively 5-10, alternatively 8-10, alternatively 3-8.

In certain aspects, the present technology relates to a compound of Formula IV:

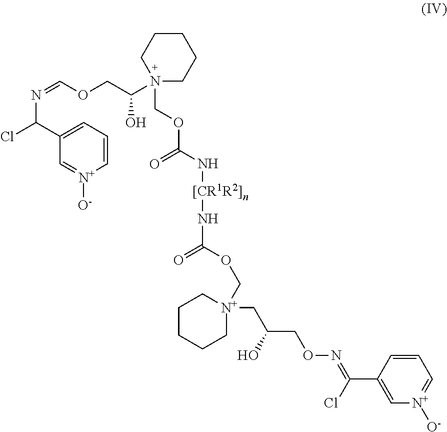

n is 1-10,
R¹ and R² are selected independently from each other and for each CR₁R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a further aspect, the present technology relates to the compound of Formula V, wherein n is alternatively 1-3, alternatively 3-5, alternatively 5-7, alternatively 7-9, alternatively 1-5, alternatively 5-10, alternatively 8-10, alternatively 3-8.

In certain aspects, the present technology relates to a compound of Formula V

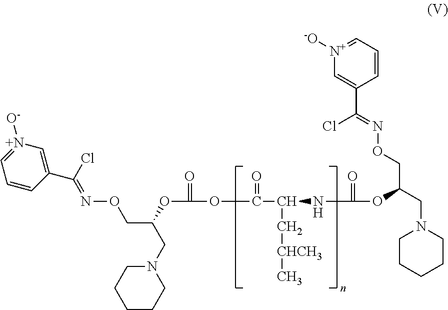

wherein n is 1-50,
or a pharmaceutically acceptable salt thereof.

In a further aspect, the present technology relates to the compound of Formula V, wherein n is alternatively 1-3, alternatively 3-5, alternatively 5-7, alternatively 7-9, alternatively 1-5, alternatively 5-10, alternatively 8-10, alternatively 3-8.

In certain aspects, the present technology relates to a compound of Formula VI:

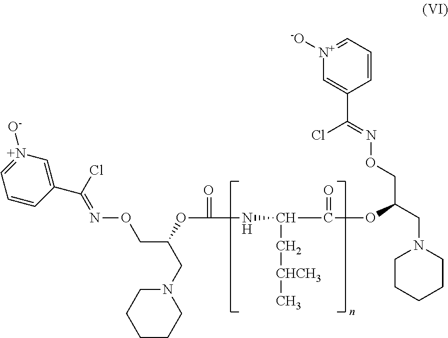

wherein n is 1-50;
or a pharmaceutically acceptable salt thereof.

In a further aspect, the present technology relates to the compound of Formula V, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In certain aspects, the present technology relates to a compound of Formula VII:

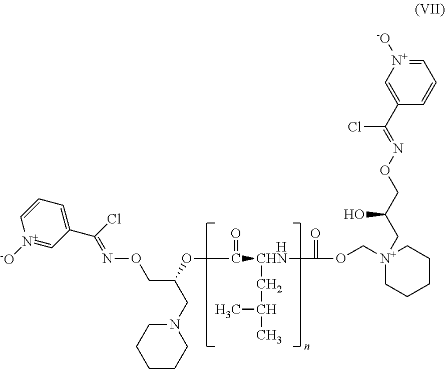

wherein n is 1-50,
or a pharmaceutically acceptable salt thereof.

In certain aspects, the present technology relates to a compound having a structure selected from:

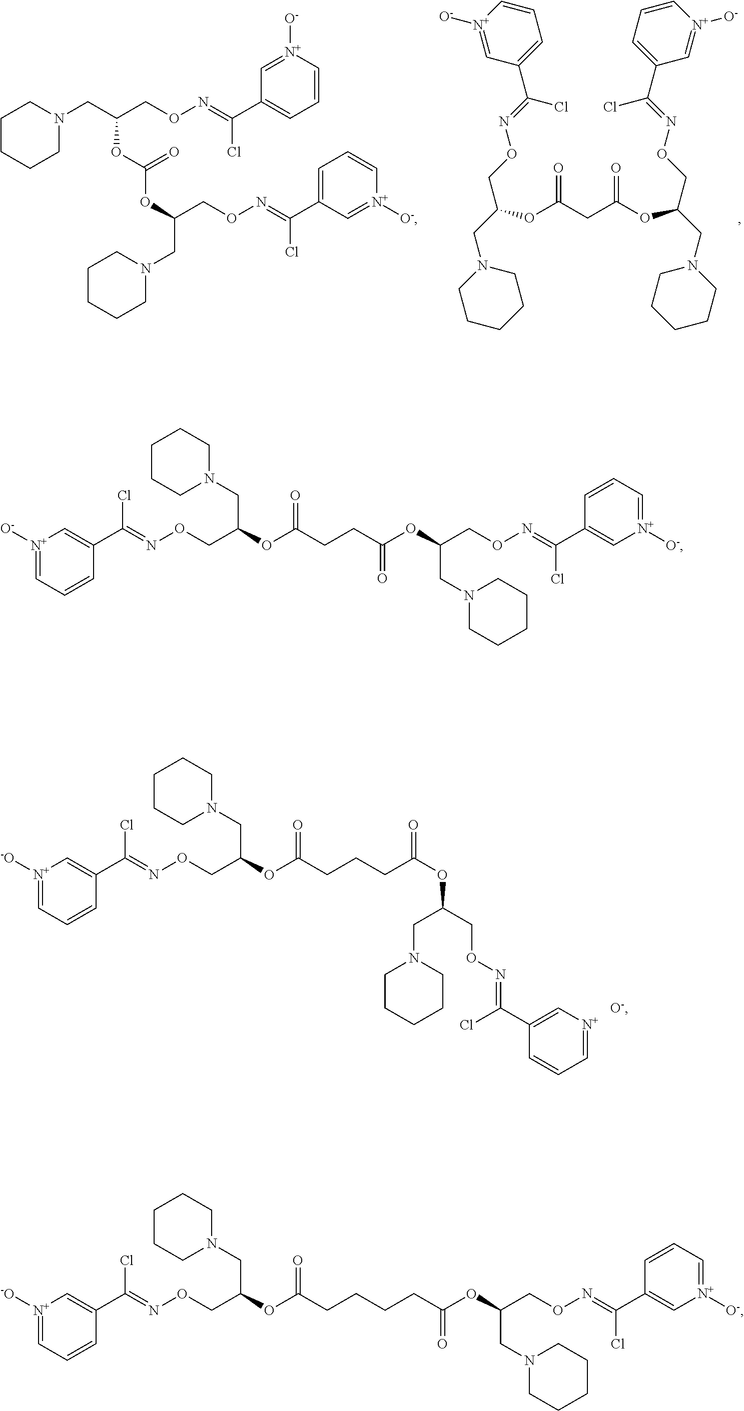

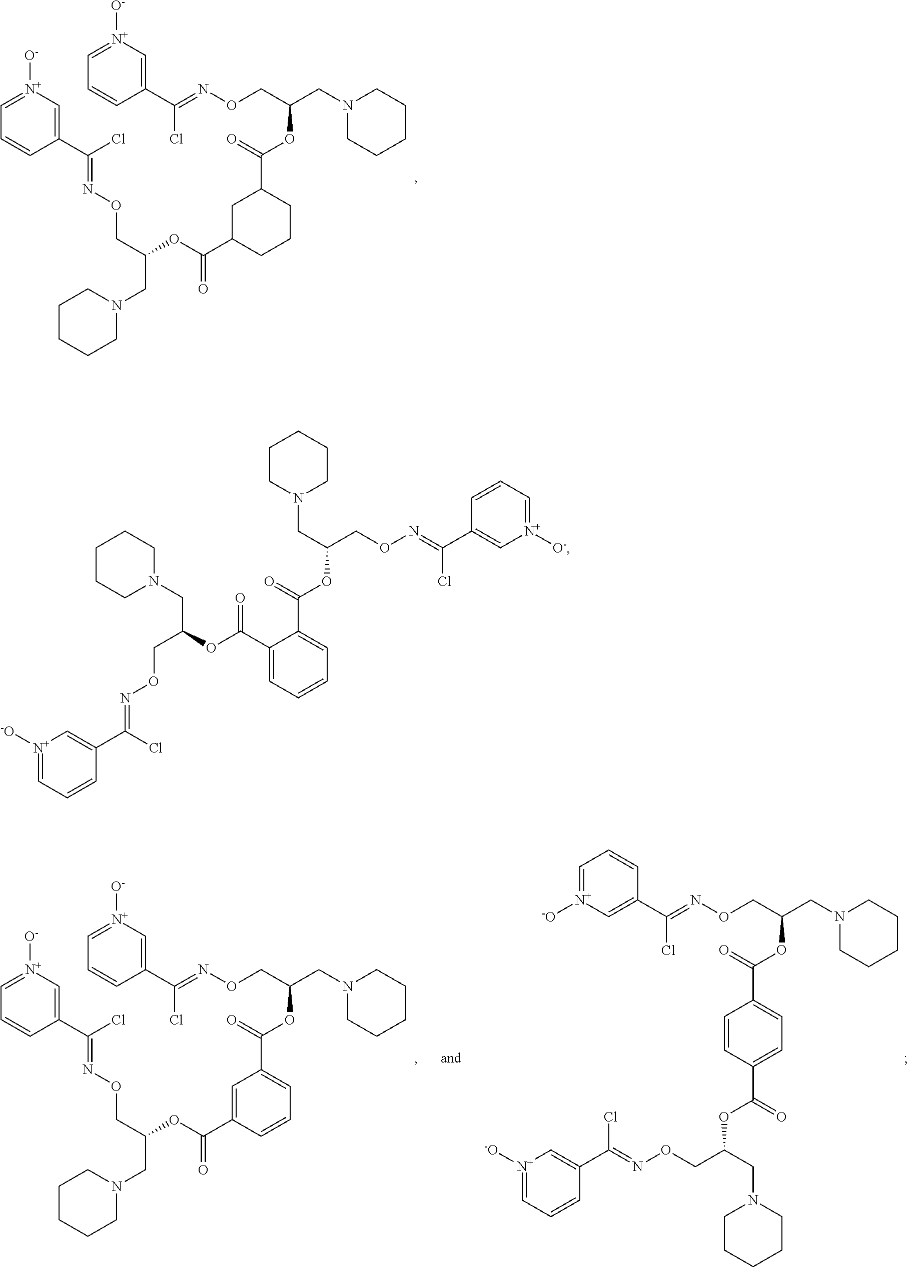

or a pharmaceutically acceptable salt thereof.

In certain aspects, the present technology relates to a compound selected from:

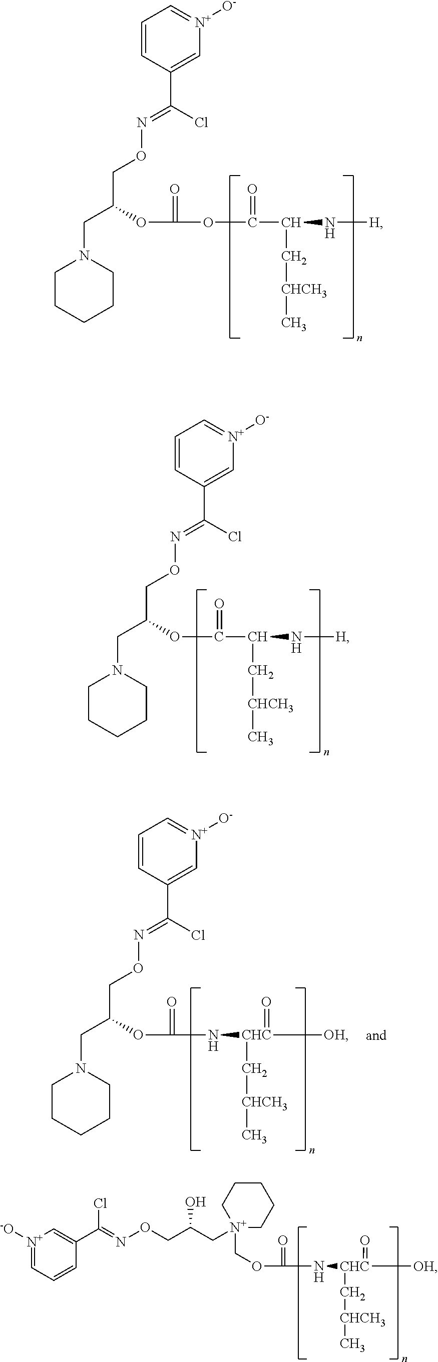

wherein n is 1-50; or a pharmaceutically acceptable salt thereof. Alternatively, n is 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In a further aspect, the present technology relates to a compound of Formula I to VII, or pharmaceutically acceptable salts thereof, wherein the pharmaceutically acceptable salt is selected from sulfate, citrate, acetate, oxalate, chloride, hydrochloride, bromide, hydrobromide, iodide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In certain aspects, the present technology relates to a composition comprising:
a therapeutically effective amount of a compound of Formula I:

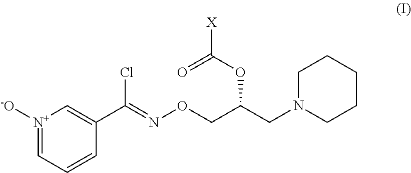

wherein X is selected from the group consisting of R, [O—R], [NR¹R²], and [NRH], where
O is oxygen, N is nitrogen, and H is hydrogen;
R, R¹ and R² are independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a further aspect, the present technology relates to a composition comprising a therapeutically effective amount of a compound of Formula I, wherein the compound is a compound having a structure of Formula IA:

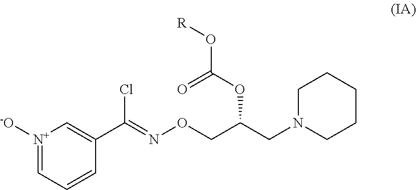

wherein R is selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a still further aspect, the present technology relates to a composition comprising a therapeutically effective amount of a compound of Formula IA, wherein the compound of Formula IA is a compound having a structure selected from:

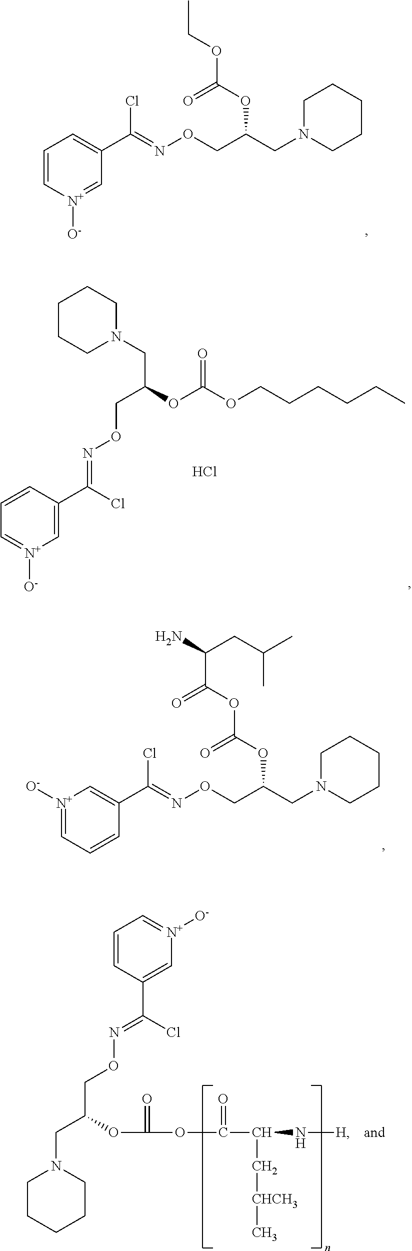

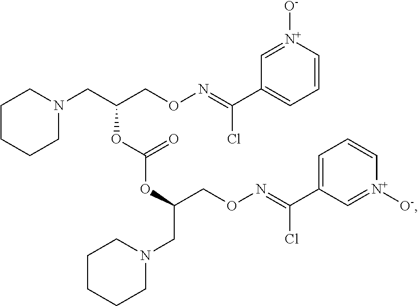

wherein n is 1-50;
or a pharmaceutically acceptable salt thereof.

In a further aspect, the present technology relates to a composition comprising a therapeutically effective amount of a compound of Formula IA, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In a further aspect, the present technology relates to a composition comprising a therapeutically effective amount of a compound of Formula I, wherein the compound of Formula I is a compound having a structure of Formula IB:

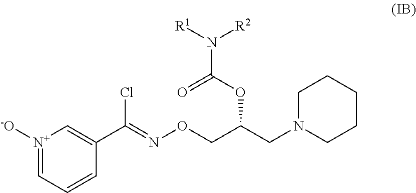

wherein R¹ and R₂ are independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a still further aspect, the present technology relates to a composition comprising a therapeutically effective amount of a compound of Formula IB, wherein the compound of Formula IB is a compound having a structure selected from:

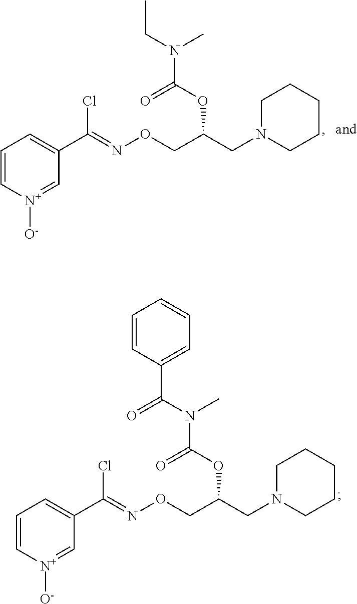 [SMILES: CCN(C)C(=O)O[C@@H](CO/N=C(\Cl)c1ccc[n+]([O-])c1)CN1CCCCC1 ; CN(C(=O)O[C@@H](CO/N=C(\Cl)c1ccc[n+]([O-])c1)CN1CCCCC1)C(=O)c1ccccc1]

or a pharmaceutically acceptable salt thereof.

In a further aspect, the present technology relates to a composition comprising a therapeutically effective amount of a compound of Formula I, wherein the compound of Formula I is a compound of Formula IC:

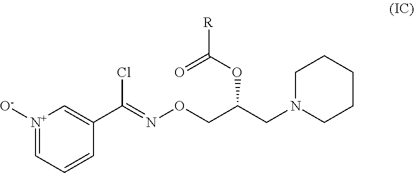

wherein R is selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a further aspect, the present technology relates to a composition comprising a therapeutically effective amount of a compound of Formula IC, wherein the compound of Formula IC is a compound having a structure selected from:

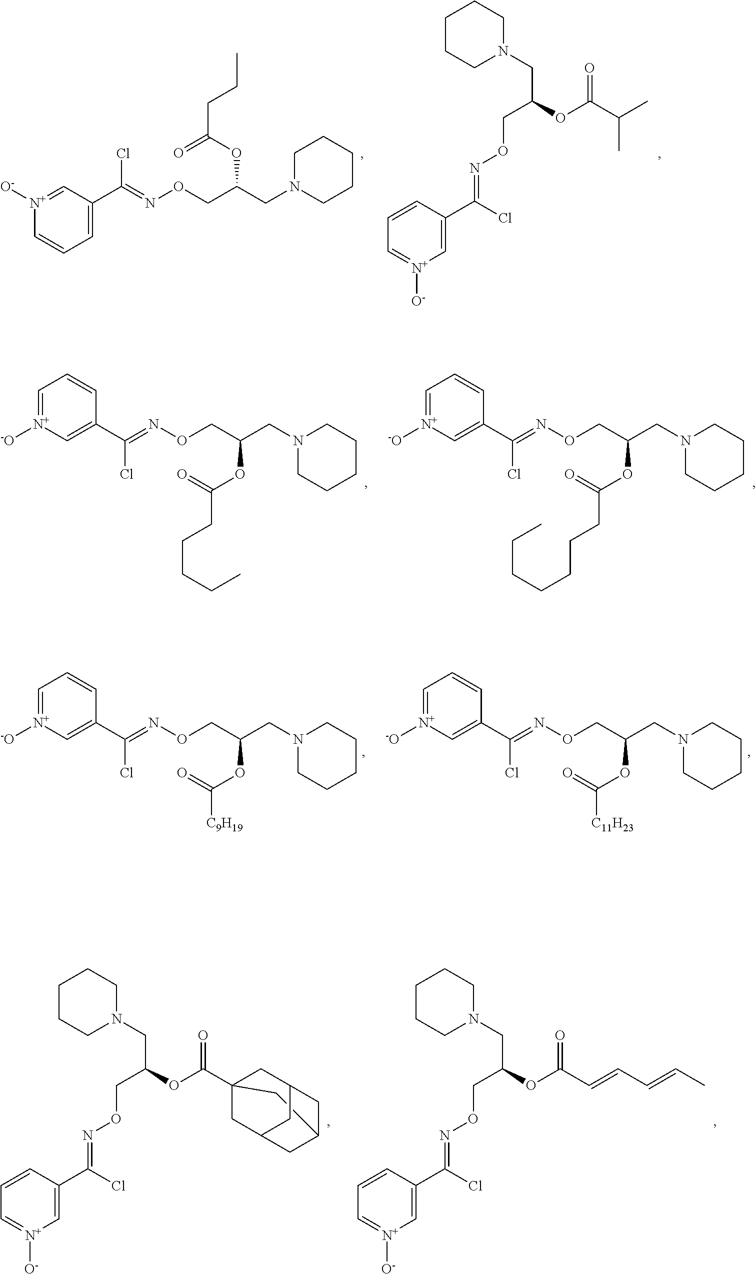

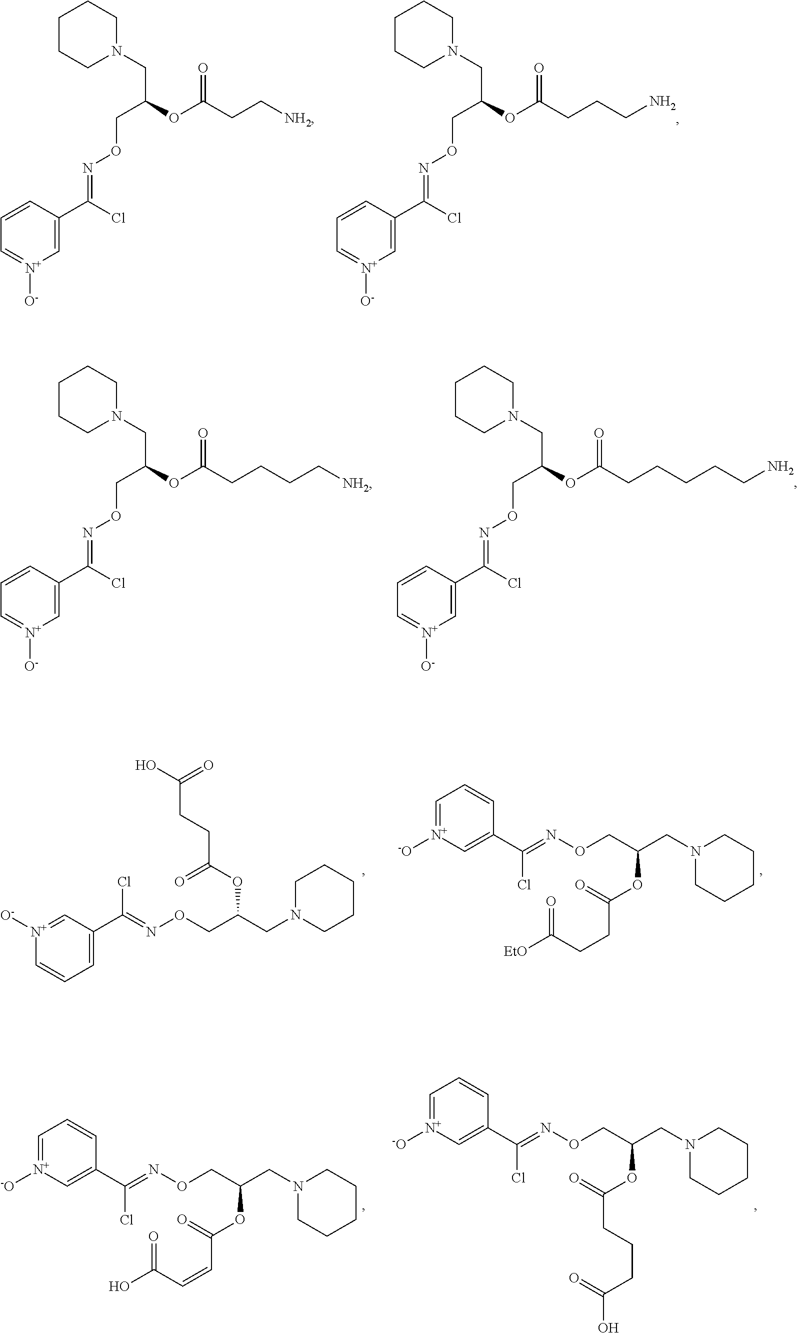

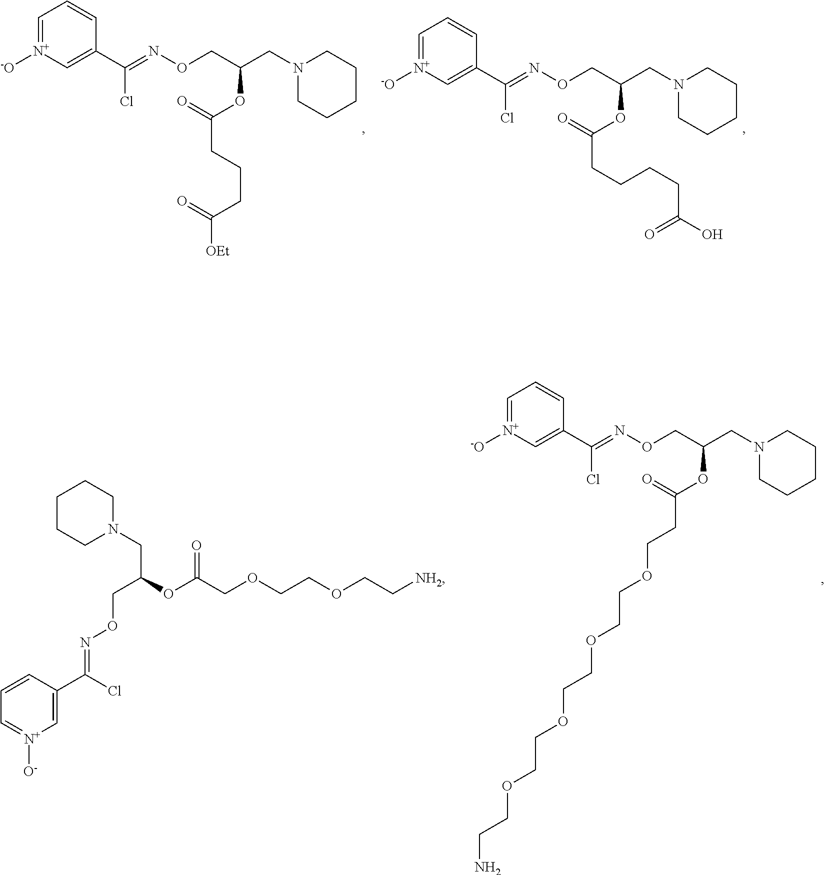

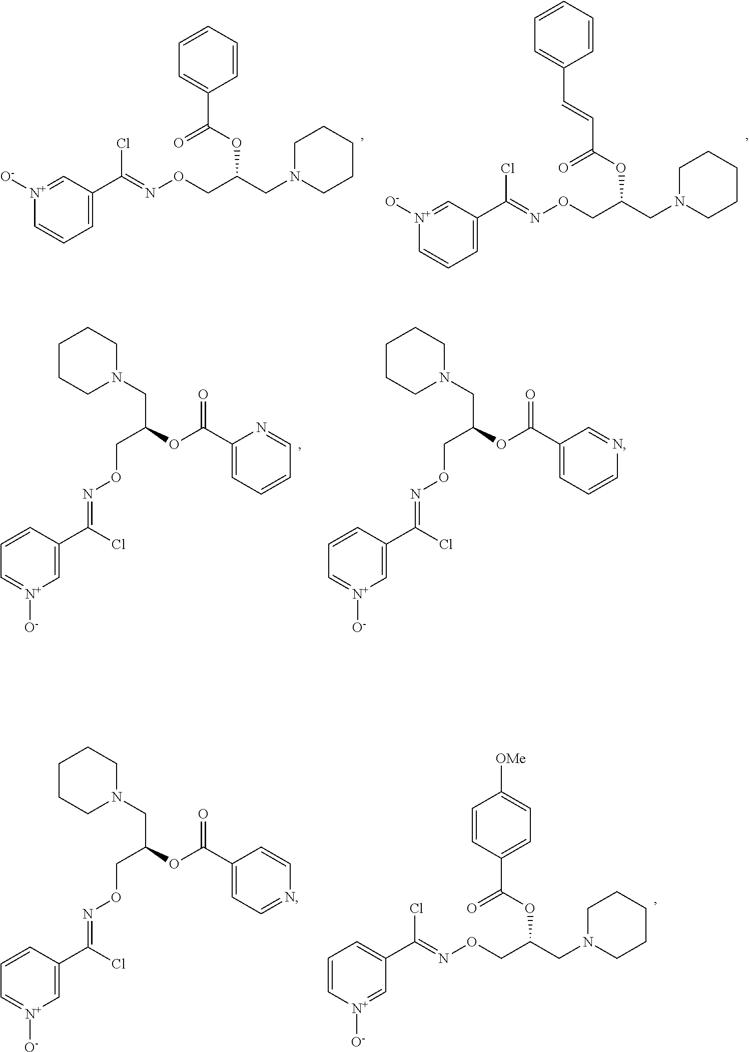

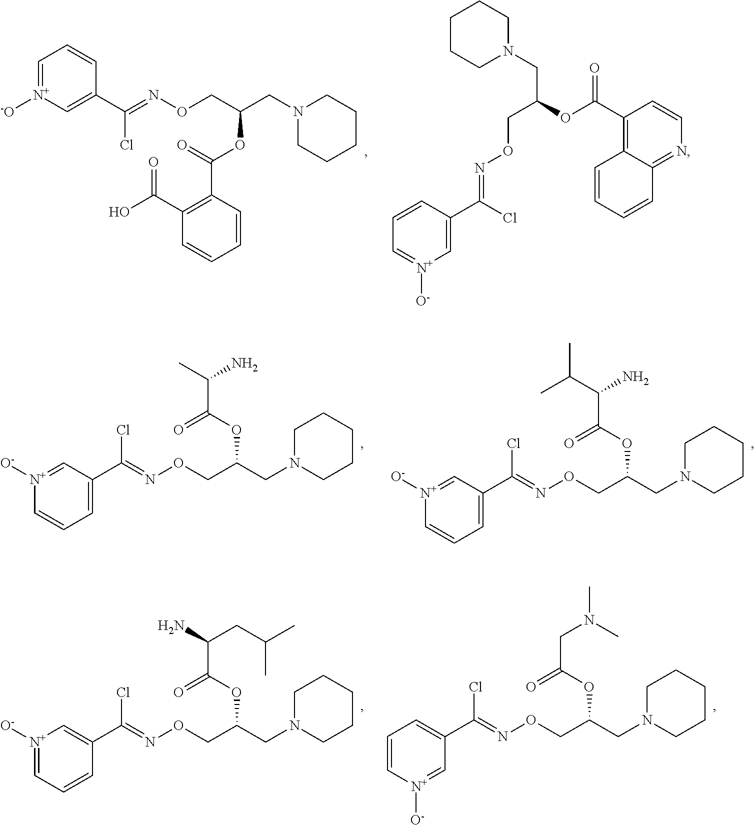

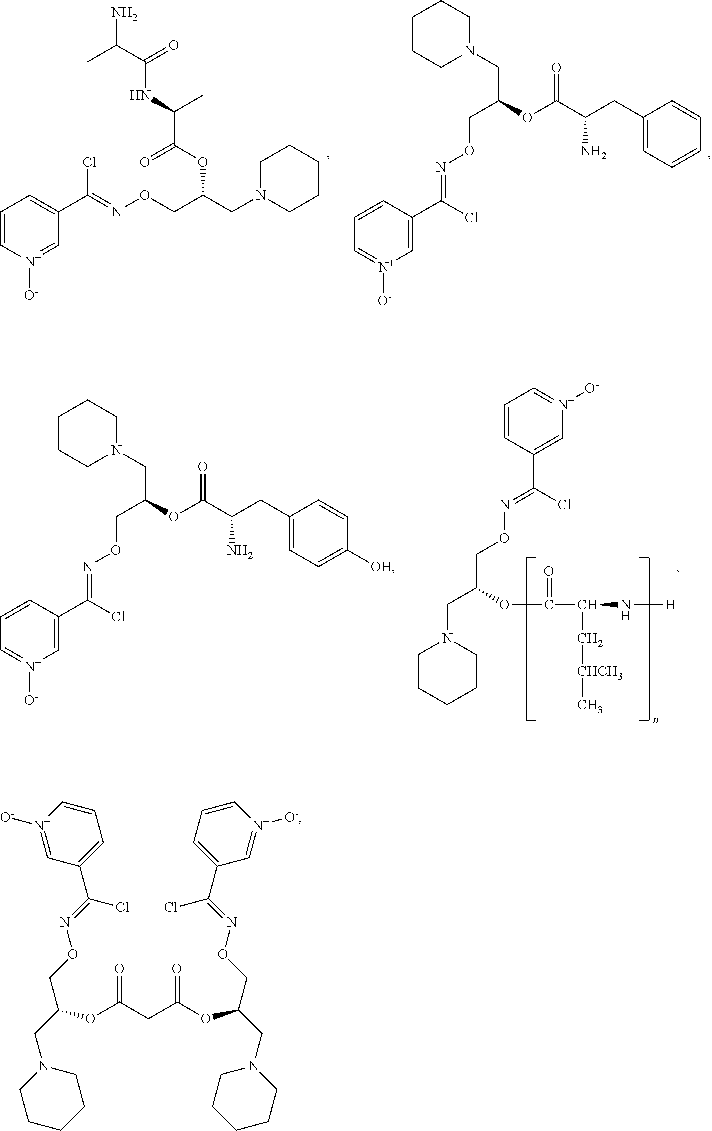

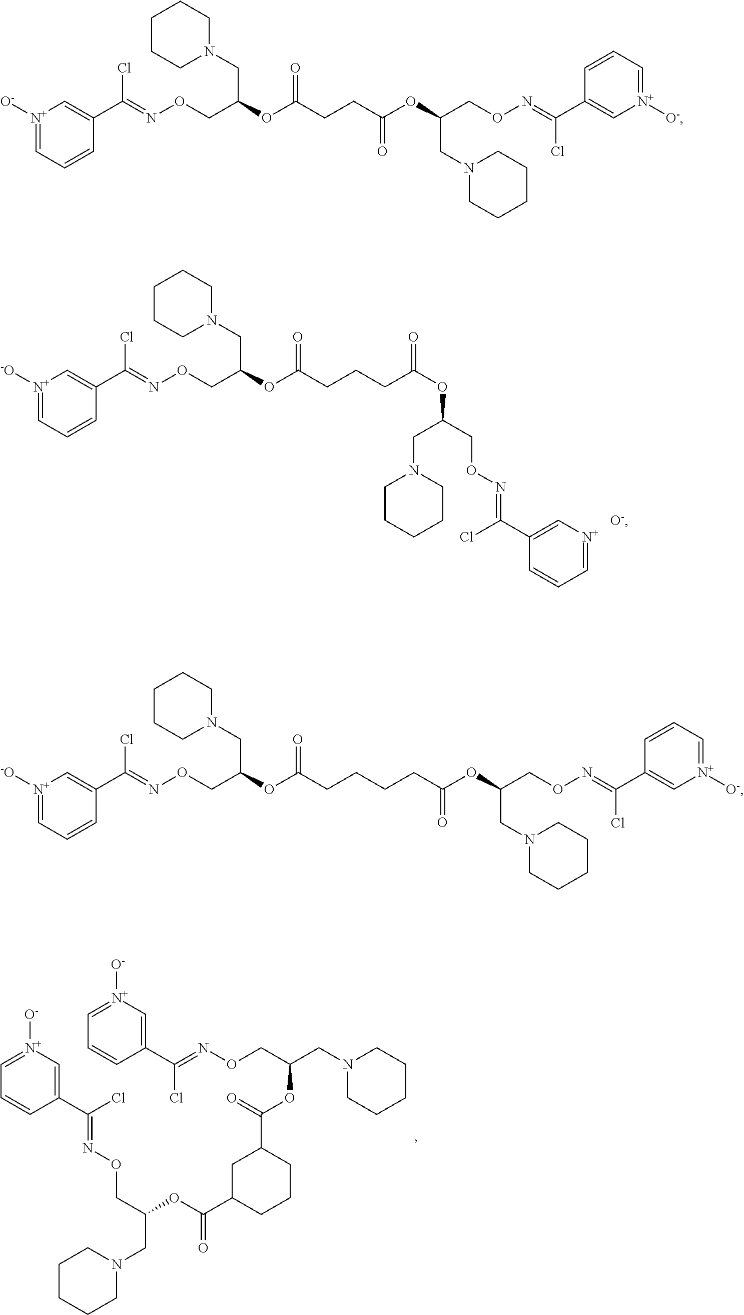

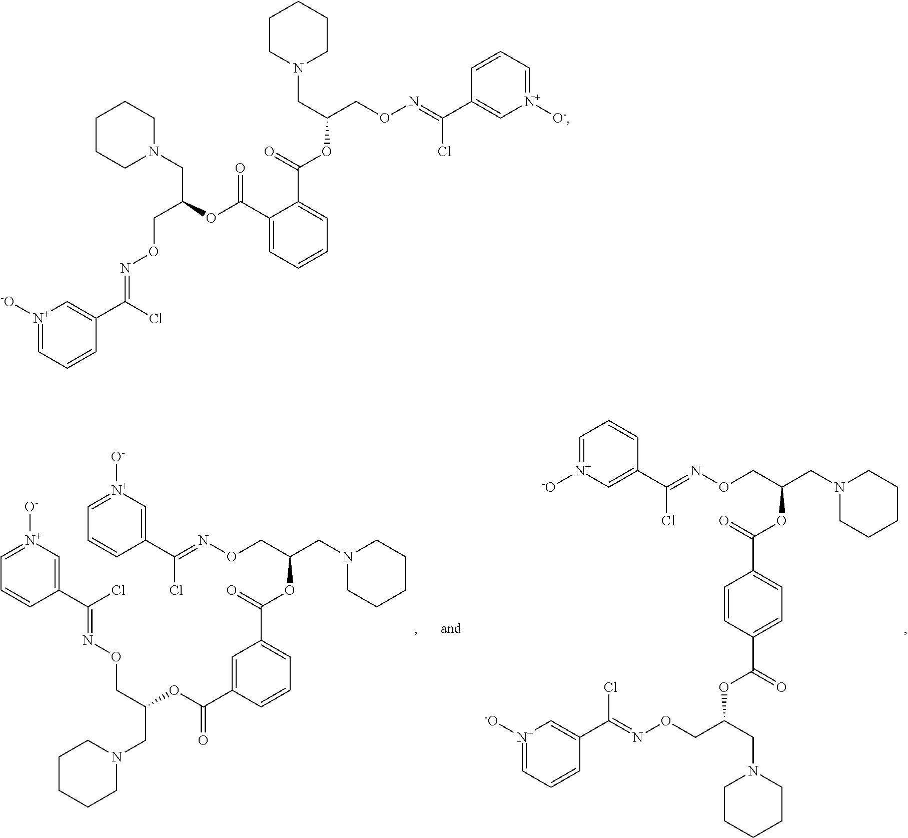

wherein n is 1-50;
or a pharmaceutically acceptable salt thereof.

In a still further aspect, the present technology relates to a composition comprising a therapeutically effective amount of a compound of Formula IC, wherein the compound of Formula IC is a compound having a structure selected from:

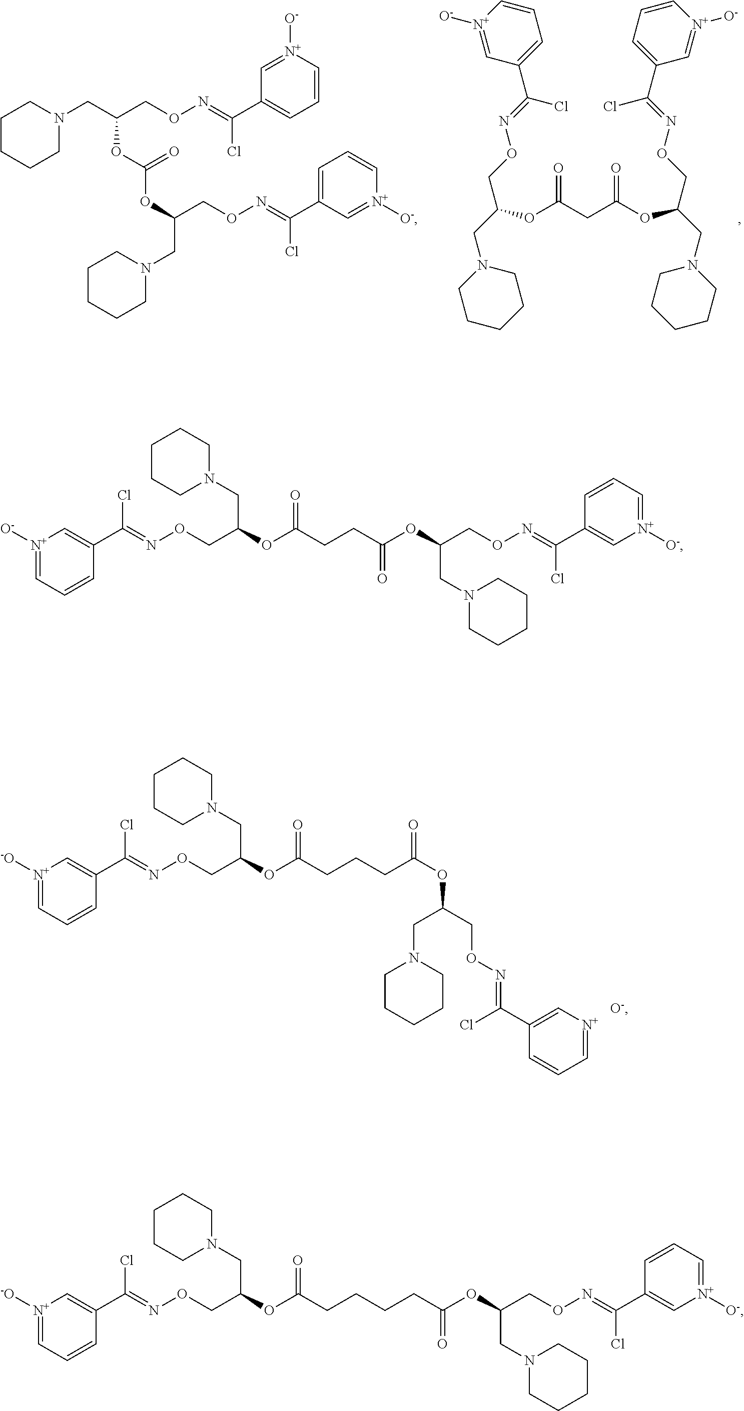

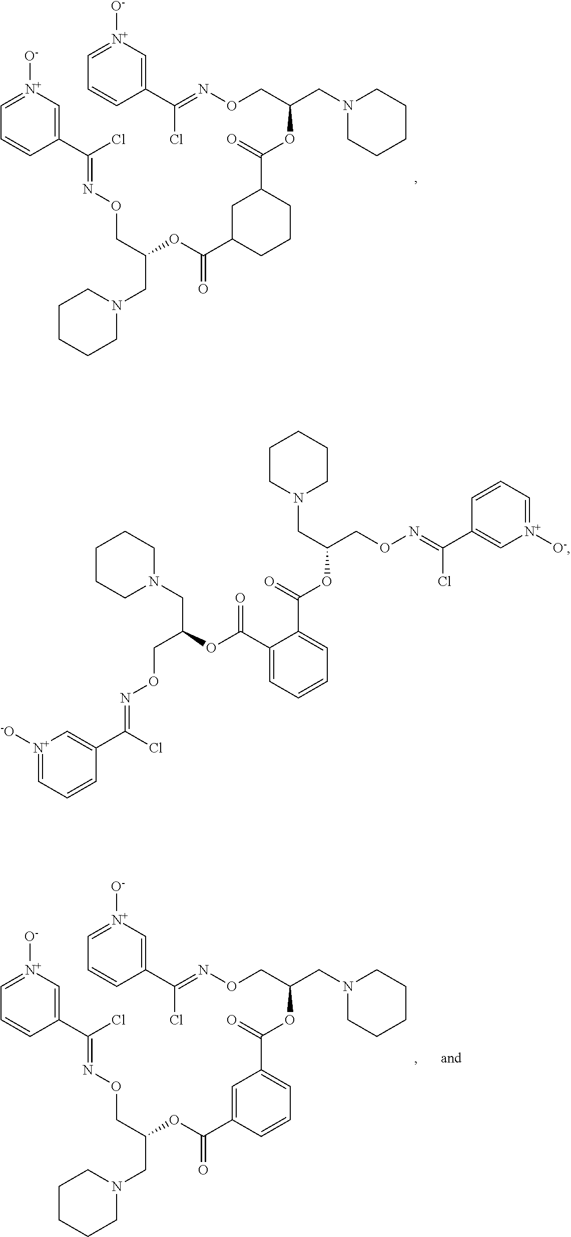

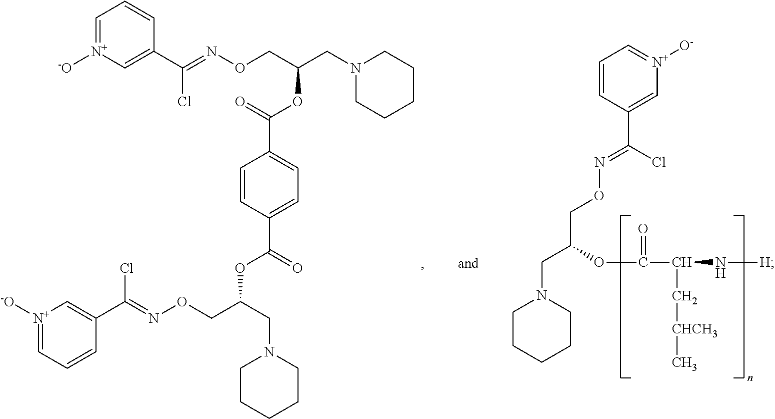

wherein n is 1-50
or a pharmaceutically acceptable salt thereof.

In a further aspect, the present technology relates to a composition comprising a therapeutically effective amount of a compound of Formula ID, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In a further aspect, the present technology relates to a composition comprising a therapeutically effective amount of a compound of Formula I, wherein the compound of Formula I is a compound of Formula ID:

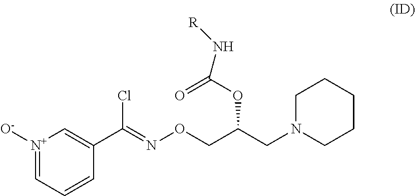

wherein R is selected from the group consisting of alkenyl, alkenyl, aminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a still further aspect, the present technology relates to a composition comprising a therapeutically effective amount of a compound of Formula ID, wherein the compound of Formula ID is a compound having a structure selected from:

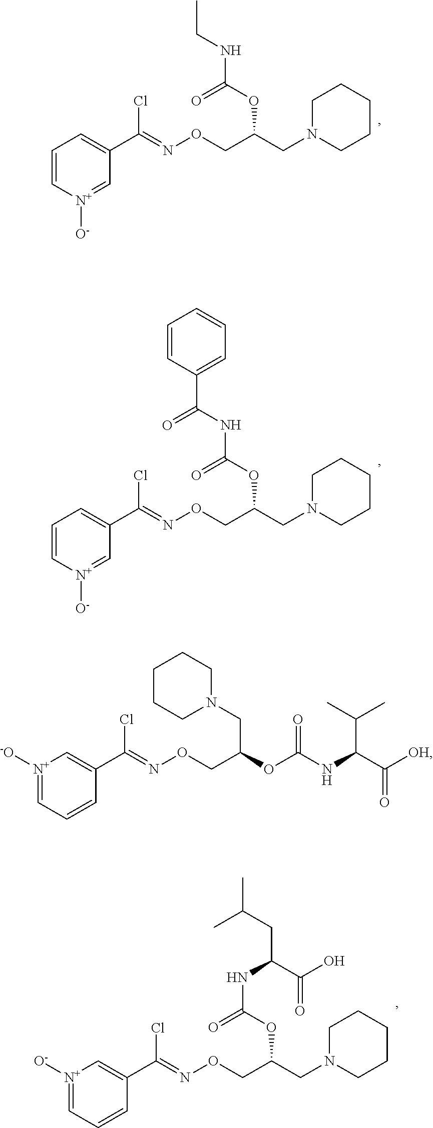

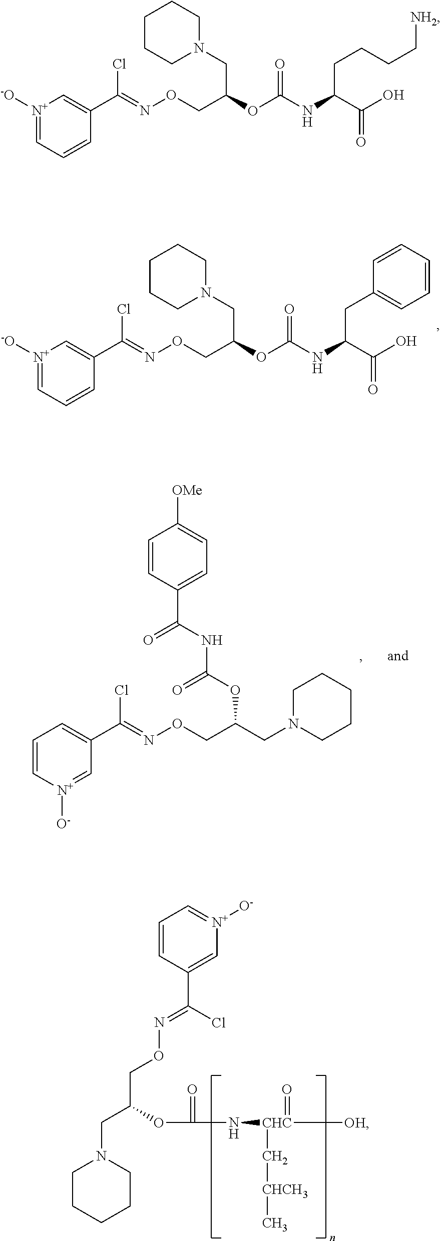

wherein n is 1-50;
or a pharmaceutically acceptable salt thereof.

In a further aspect, the present technology relates to a composition comprising a therapeutically effective amount of a compound of Formula ID, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In a still further aspect, the present technology relates to a composition comprising:
a therapeutically effective amount of a compound of Formula II:

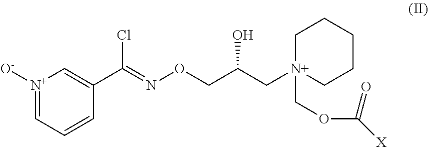

wherein X is selected from the group consisting of R, [O—R], [NR¹R²], and [NRH]
wherein O is oxygen, N is nitrogen, H is hydrogen, and
R, R¹ and R² are independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol
or a pharmaceutically acceptable salt thereof.

In a still further aspect, the present technology relates to a composition comprising a therapeutically effective amount of a compound of Formula II, wherein the compound of Formula II is selected from:

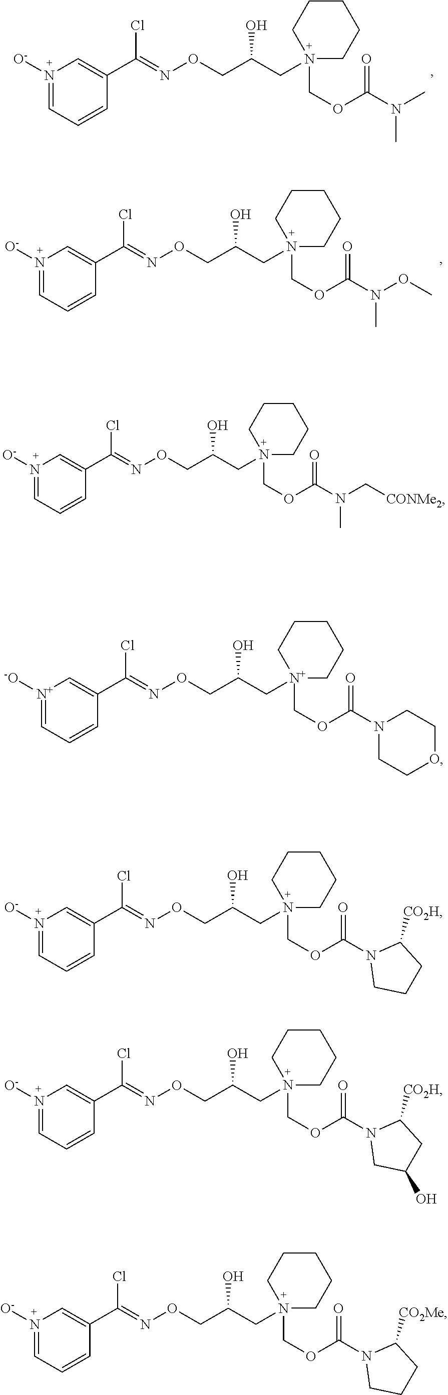

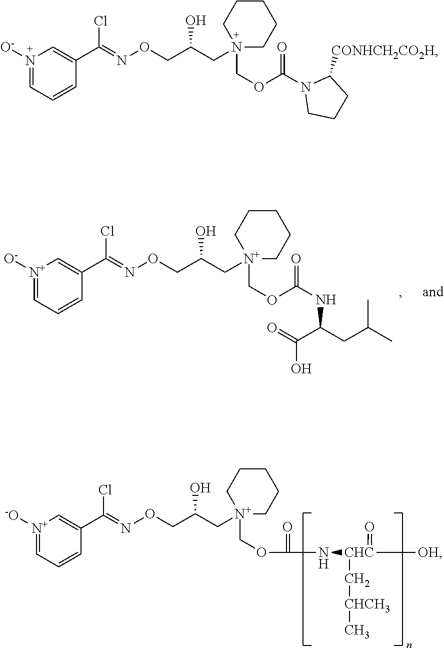

wherein n is 1-50;
or a pharmaceutically acceptable salt thereof.

In a further aspect, the present technology relates to a composition comprising a therapeutically effective amount of a compound of Formula II, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In certain aspects, the present technology relates to a composition comprising a therapeutically effective amount of a compound having a structure selected from:

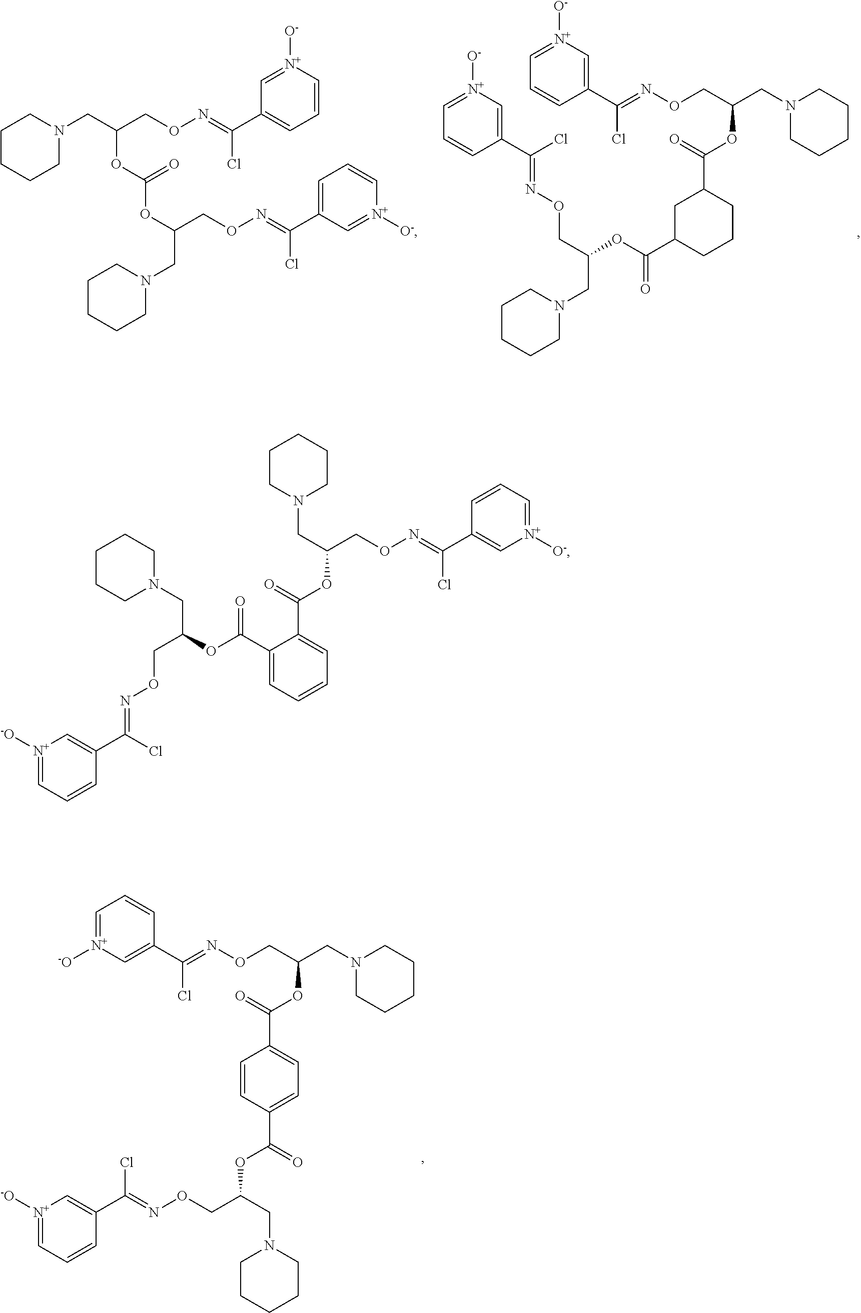

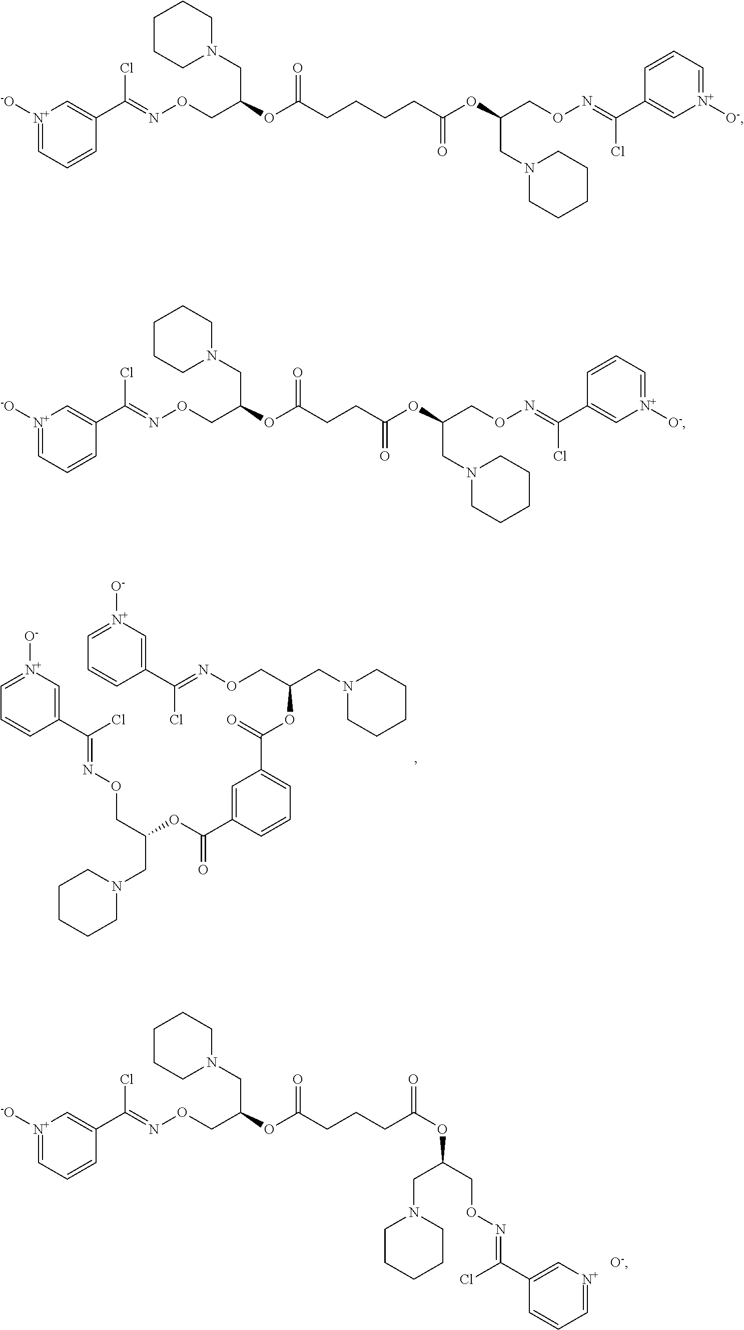

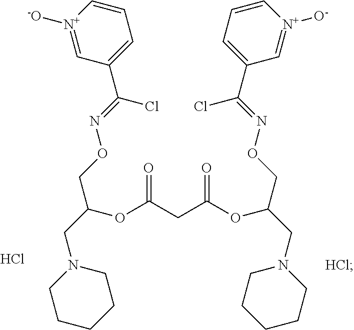

or a pharmaceutically acceptable salt thereof.

In certain aspects, the present technology relates to a composition comprising a therapeutically effective amount of a compound selected from:

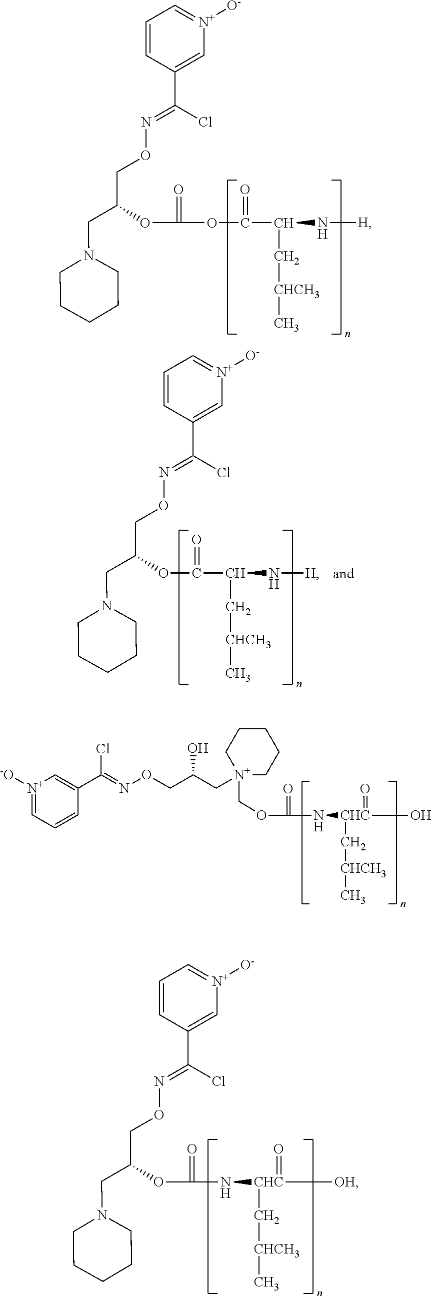

wherein n is 1-50; or a pharmaceutically acceptable salt thereof. Alternatively, n is 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In certain aspects, the present technology relates to a composition comprising a therapeutically effective amount of a compound of Formula III:

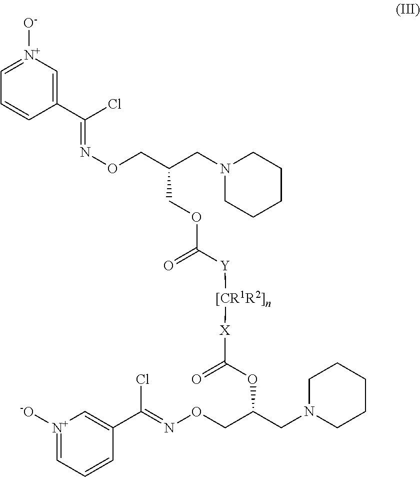

wherein X is selected from the group consisting of O, N, or absent,
Y is selected from the group consisting of O, N, or absent,
n is 1-10,
R¹ and R² are selected independently from each other and for each CR¹R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a further aspect, the present technology relates to a composition comprising a therapeutically effective amount of a compound of Formula IV, wherein n is alternatively 1-3, alternatively 3-5, alternatively 5-7, alternatively 7-9, alternatively 1-5, alternatively 5-10, alternatively 8-10, alternatively 3-8.

In certain aspects, the present technology relates to a composition comprising a therapeutically effective amount of a compound of Formula IV:

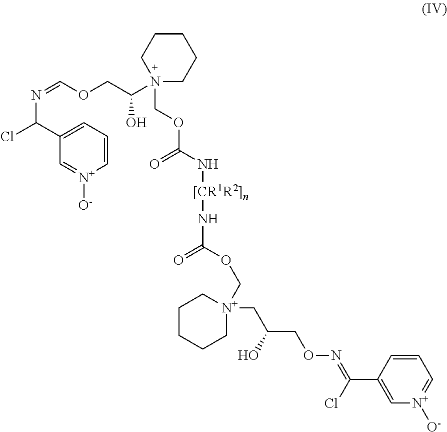

n is 1-10,
R¹ and R² are selected independently from each other and for each CR₁R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a further aspect, the present technology relates to a composition comprising a therapeutically effective amount of s compound of Formula IV, wherein n is alternatively 1-3, alternatively 3-5, alternatively 5-7, alternatively 7-9, alternatively 1-5, alternatively 5-10, alternatively 8-10, alternatively 3-8.

In certain aspects, the present technology relates to a composition comprising a therapeutically effective amount of a compound of Formula V

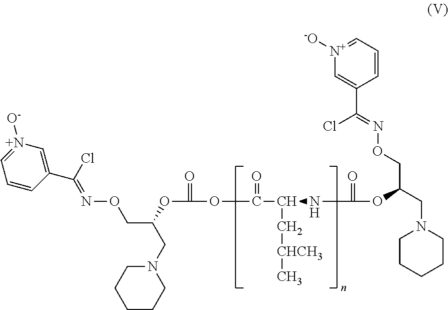

wherein n is 1-50,
or a pharmaceutically acceptable salt thereof.

In a further aspect, the present technology relates to a composition comprising a therapeutically effective amount of a compound of Formula V, wherein n is alternatively 1-3, alternatively 3-5, alternatively 5-7, alternatively 7-9, alternatively 1-5, alternatively 5-10, alternatively 8-10, alternatively 3-8.

In certain aspects, the present technology relates to a composition comprising a therapeutically effective amount of a compound of Formula VI:

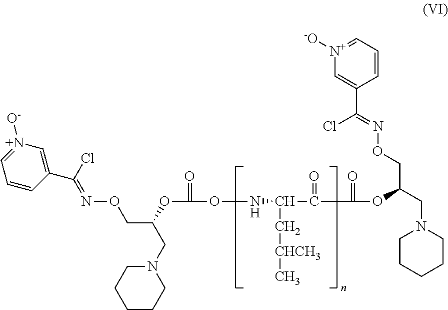

wherein n is 1-50;
or a pharmaceutically acceptable salt thereof.

In a further aspect, the present technology relates to a composition comprising a therapeutically effective amount of a compound of Formula VI, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In certain aspects, the present technology relates to a composition comprising a therapeutically effective amount of a compound of Formula VII:

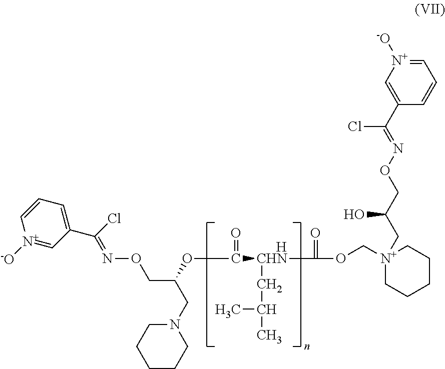

wherein n is 1-50, or a pharmaceutically acceptable salt thereof.

In a further aspect, the present technology relates to a composition comprising a therapeutically effective amount of a compound of Formula VII, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In a still further aspect, the present technology relates to a composition comprising a therapeutically effective amount of a compound described above, wherein the pharmaceutically acceptable salt of the compound of Formula I to VII selected from: sulfate, citrate, acetate, oxalate, chloride, hydrochloride, bromide, hydrobromide, iodide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate. In a still further aspect, the pharmaceutically acceptable salt of the compound of Formula I to VII is citrate.

In a still further aspect, the present technology relates to a composition comprising a therapeutically effective amount of a compound described above, wherein the compound is present as a stereoisomer of said compound. In a still further aspect, wherein the compound of Formula I is present as a stereoisomer and comprises a sub-structure selected from (+)-(R)—N-[2-hydroxy-3-(1-piperidinyl)-propoxy]-pyridine-1-oxide-3-carboximidoyl chloride, and (−)-(S)—N-[2-hydroxy-3-(1-piperidinyl)-propoxy]-pyridine-1-oxide-3-carboximidoyl chloride, (Z)—(R)—N-[2-hydroxy-3-(1-piperidinyl)-propoxy]-pyridine-1-oxide-3-carboximidoyl chloride, (E)-(R)—N-[2-hydroxy-3-(1 piperidinyl)-propoxy]-pyridine-1-oxide-3-carboximidoyl chloride, (Z)—(S)—N-[2-hydroxy-3-(1-piperidinyl)-propoxy]-pyridine-1-oxide-3-carboximidoyl chloride; and (E)-(S)—N-[2-hydroxy-3-(1-piperidinyl)-propoxy]-pyridine-1-oxide-3-carboximidoyl chloride.

In a still further aspect, the present technology relates to a composition comprising a therapeutically effective amount of a compound of Formula I to Formula VII, wherein the compound of Formula I to Formula VII is present in a form selected from polymorphs, crystals, and combinations thereof.

In a still further aspect, the present technology relates to a composition comprising a therapeutically effective amount of a compound of Formula I to Formula VII, wherein the composition further comprises at least one excipient. In a still further aspect, the at least one excipient is selected from antiadherents, binders, coatings, disintegrants, gel forming agents, fillers, flavors and colors, glidants, lubricants, preservatives, sorbents and sweeteners.

In a still further aspect, the present technology relates to a composition comprising a therapeutically effective amount of a compound of Formula I to Formula VII, wherein the composition further comprises at least one additional active pharmaceutical ingredient. In certain aspects, the at least one additional active pharmaceutical ingredient is selected from bimoclomol, miglustat, eliglustat, N-acetyl-L-leucine and 2-hydroxypropyl-β-cyclodextrin, and combinations thereof. In a still further aspect, the at least one additional active pharmaceutical ingredient is selected from miglustat, N-acetyl-L-leucine, and combinations thereof.

In a still further aspect, the present technology relates to a composition comprising a therapeutically effective amount of a compound of Formula I to Formula VII, wherein further the composition comprises at least two additional active pharmaceutical ingredients, wherein the at least two additional active pharmaceutical ingredients is a combination of miglustat and N-acetyl-L-leucine.

In a still further aspect, the present technology relates to a composition comprising a therapeutically effective amount of a compound of Formula I to Formula VII, wherein the composition is formulated for oral administration.

In a still further aspect, the present technology relates to a composition comprising a therapeutically effective amount of a compound of Formula I to Formula VII, wherein the composition is a solid oral dosage formulation. In a still further aspect, the solid oral dosage formulation is selected from a sublingual, a gummy, a chewable tablet, a rapidly dissolving tablet, a tablet, a capsule, a caplet, a troche, a lozenge, an oral powder, a thin strip, an oral thin film (OTF), an oral strip, and a rectal film.

In a still further aspect, the present technology relates to a composition comprising a therapeutically effective amount of a compound of Formula I to Formula VII, wherein the composition is a liquid oral dosage formulation. In a still further aspect, the liquid oral dosage formulation is selected from syrups, emulsions, solutions, gels and suspensions. In certain aspects, the liquid oral solution is added to a food item such as applesauce, water, milk, juice or similar.

In a still further aspect, the present technology relates to a composition comprising a therapeutically effective amount of a compound of Formula I to Formula VII, wherein the therapeutically effective amount of the compound of Formula I ranges from about 1 to about 5000 mg per day. In a still further aspect, the therapeutically effective amount ranges from about 50 mg to about 2500 mg, alternatively about 100 mg to about 2000 mg, alternatively about 200 mg to about 1500 mg, alternatively about 500 mg to about 1000 mg per day.

In a still further aspect, the present technology relates to a composition comprising a therapeutically effective amount of a compound of Formula I to Formula VII, wherein the therapeutically effective amount of the compound of Formula I is administered as a unit dose. In certain aspects, the therapeutically effective amount of the compound of Formula I to Formula VII is administered in 1-3 unit doses. In certain aspects, the therapeutically effective amount of the compound of Formula I to Formula VII is administered in 1-2 unit doses; alternatively, 1 unit dose.

In certain aspects, the present technology relates to an extended-release composition comprising:
a therapeutically effective amount of a compound of Formula I:

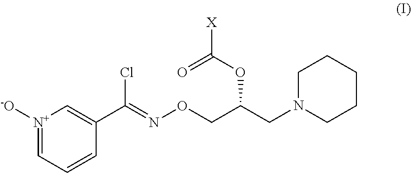

wherein X is selected from the group consisting of R, [O—R], [NR¹R²], and [NRH], where
O is oxygen, N is nitrogen, and H is hydrogen;
R, R¹ and R² are independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof; and
a release controlling agent.

In a still further aspect, the present technology relates to an extended-release composition comprising therapeutically effective amount of a compound of Formula I, wherein the compound is a compound having a structure of Formula IA:

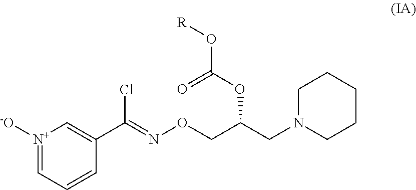

wherein R is selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a still further aspect, the present technology relates to an extended-release composition comprising a therapeutically effective amount of a compound of Formula IA, wherein the compound of Formula IA is a compound having a structure selected from:

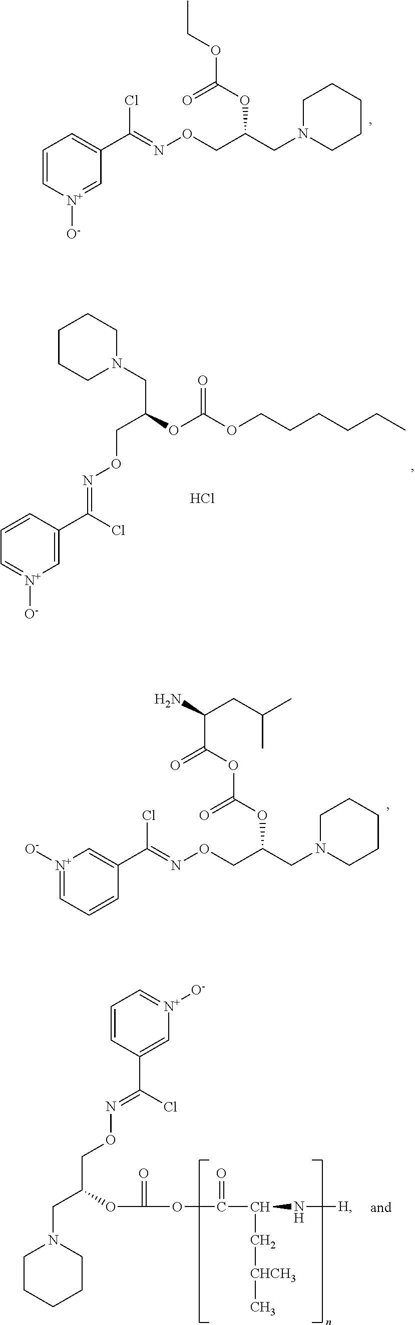

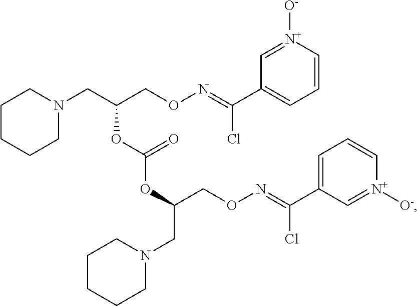

wherein n is 1-50;
or a pharmaceutically acceptable salt thereof.

In a further aspect, the present technology relates to an extended-release composition comprising a therapeutically effective amount of a compound of Formula IA, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In a further aspect, the present technology relates to an extended-release composition comprising a therapeutically effective amount of a compound of Formula I, wherein the compound of Formula I is a compound having a structure of Formula IB:

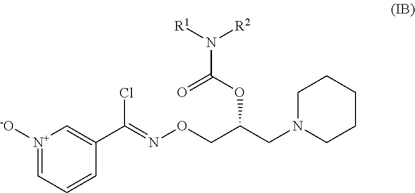

wherein R¹ and R₂ are independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a still further aspect, the present technology relates to an extended-release composition comprising a therapeutically effective amount of a compound of Formula IB, wherein the compound of Formula IB is a compound having a structure selected from:

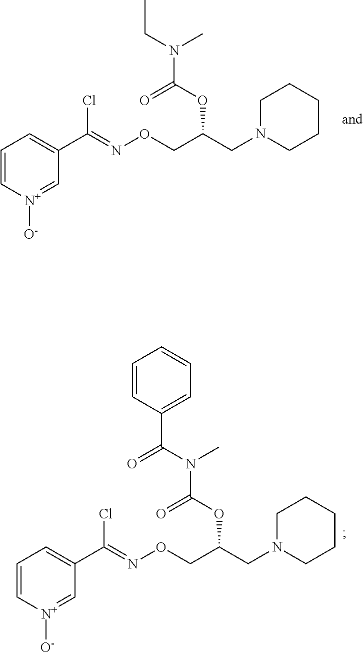 [SMILES: CCN(C)C(=O)O[C@@H](CO/N=C(\Cl)c1ccc[n+]([O-])c1)CN1CCCCC1 ; CN(C(=O)O[C@@H](CO/N=C(\Cl)c1ccc[n+]([O-])c1)CN1CCCCC1)C(=O)c1ccccc1]

or a pharmaceutically acceptable salt thereof.

In a further aspect, the present technology relates to an extended-release composition comprising a therapeutically effective amount of a compound of Formula I, wherein the compound of Formula I is a compound of Formula IC:

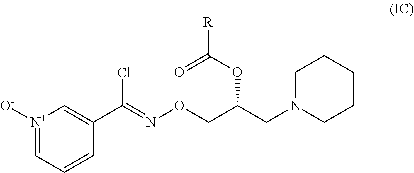

wherein R is selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a still further aspect, the present technology relates to an extended-release composition comprising a therapeutically effective amount of a compound of Formula IC, wherein the compound of Formula IC is a compound having a structure selected from:

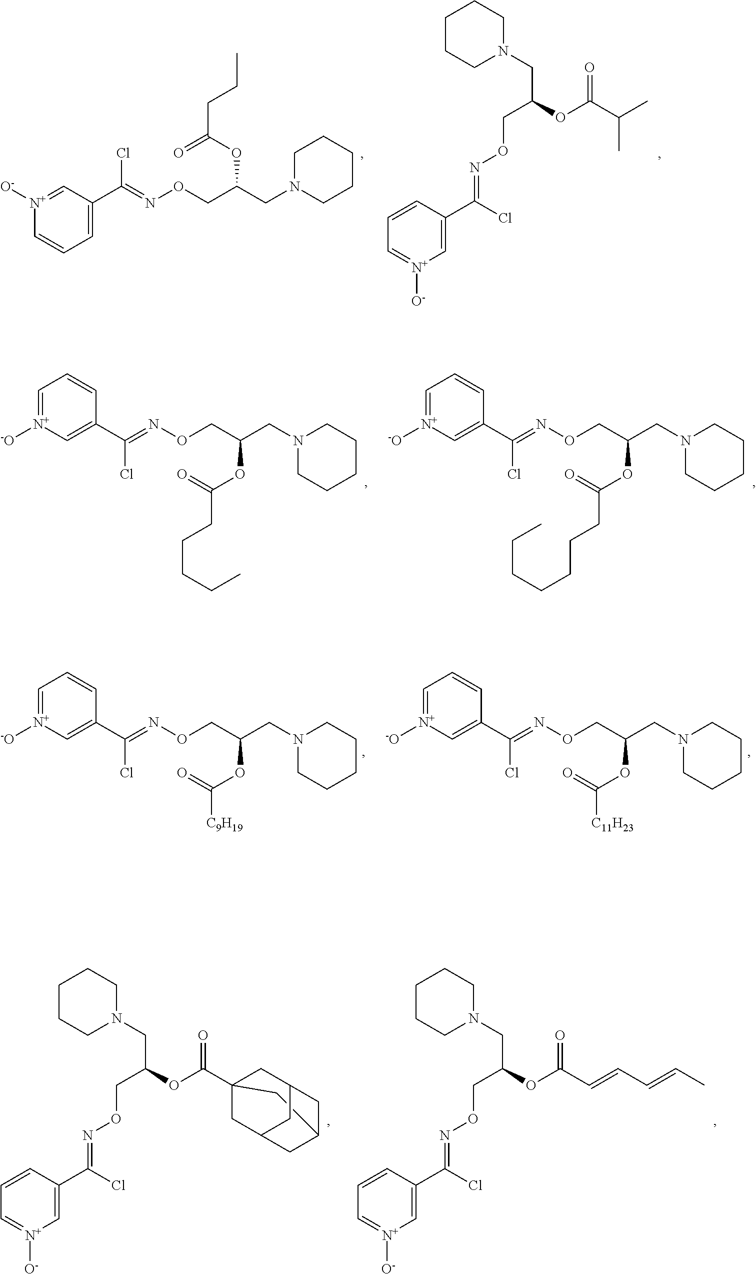

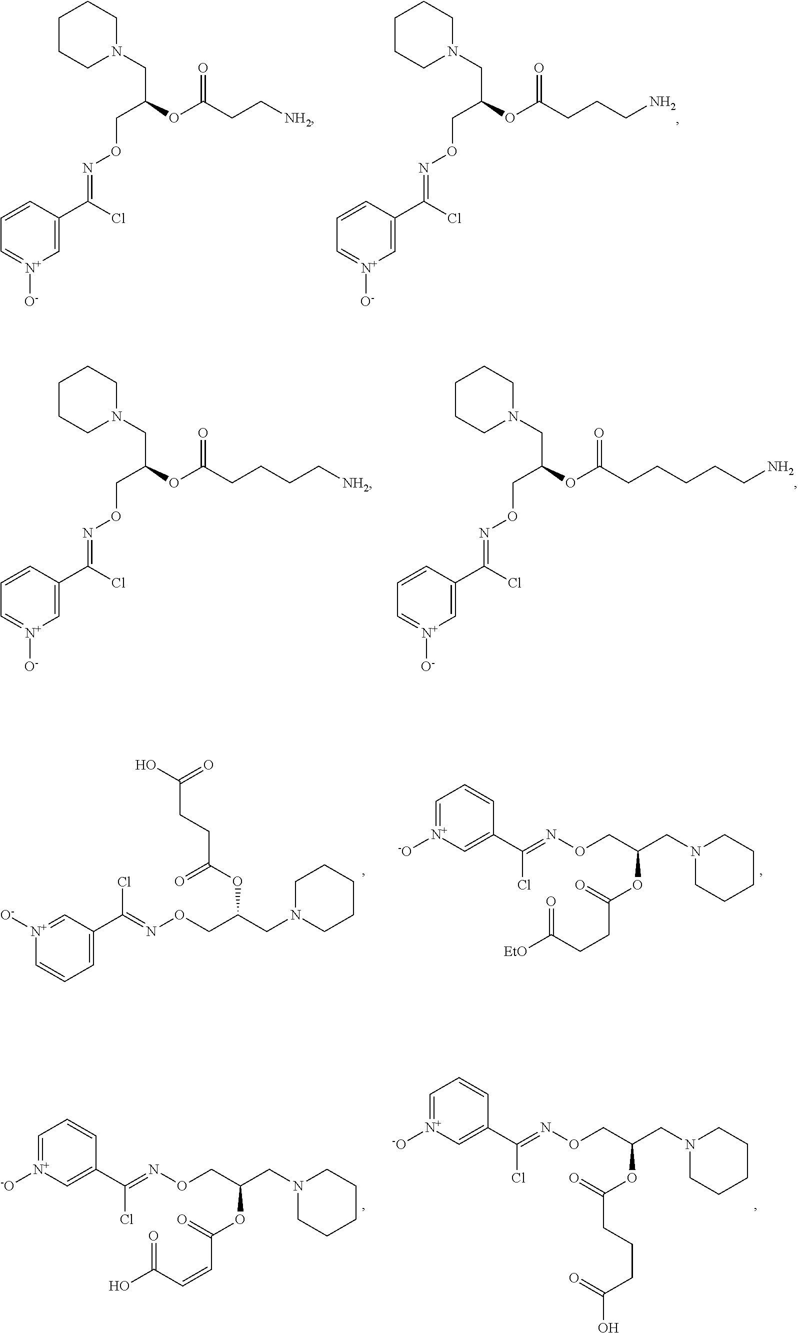

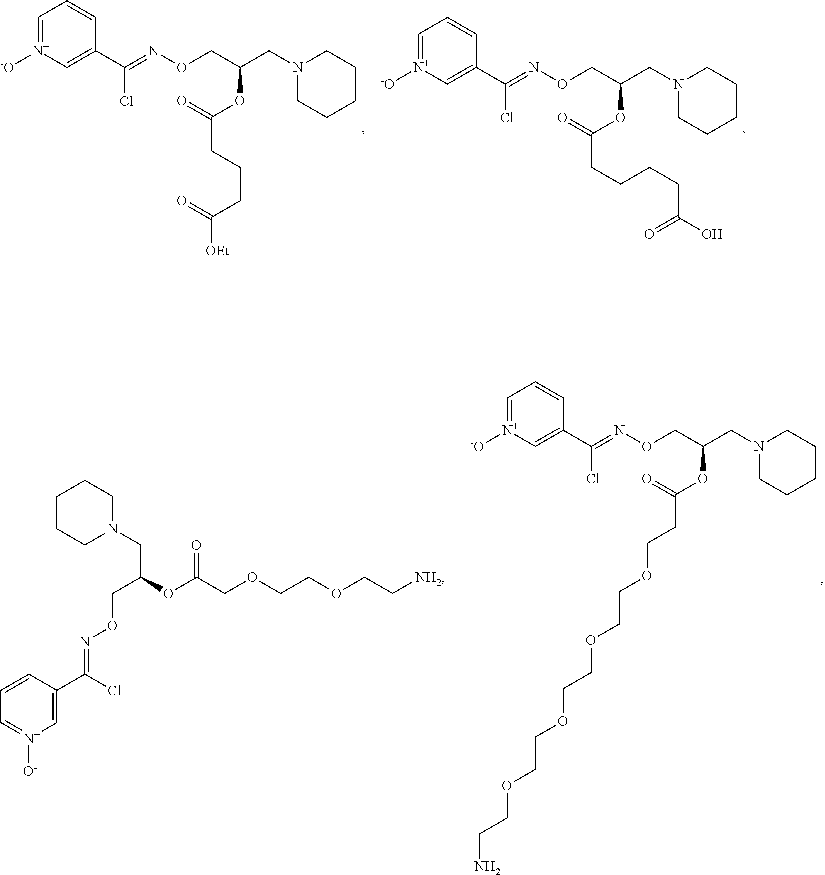

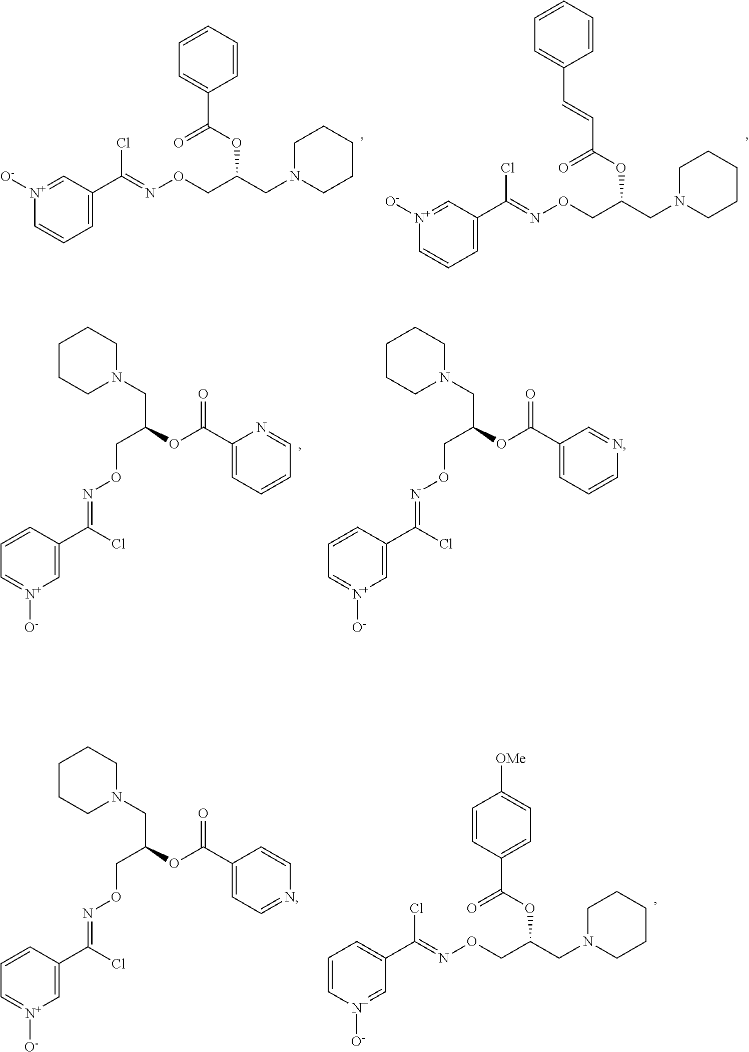

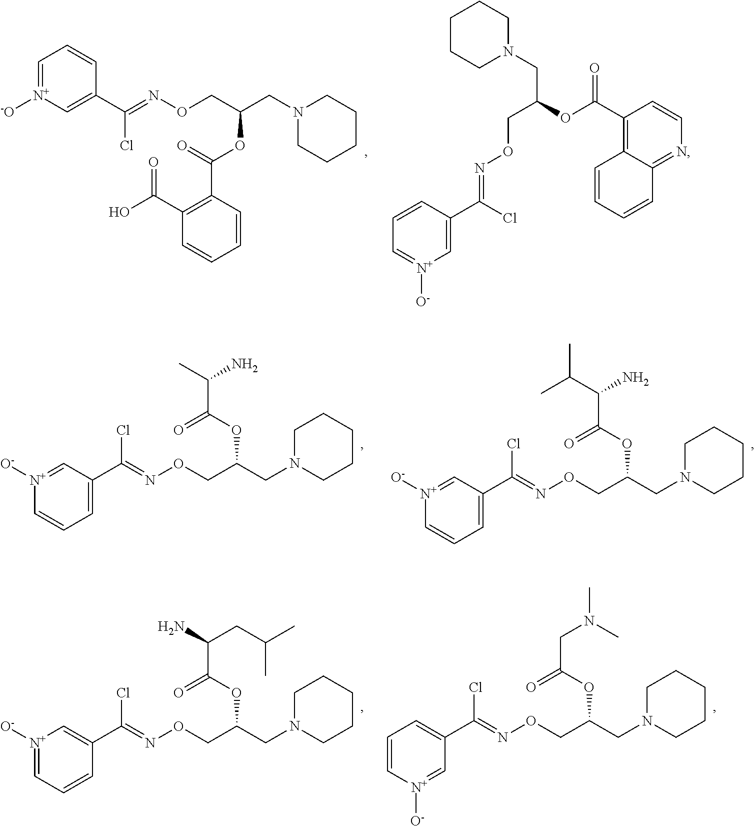

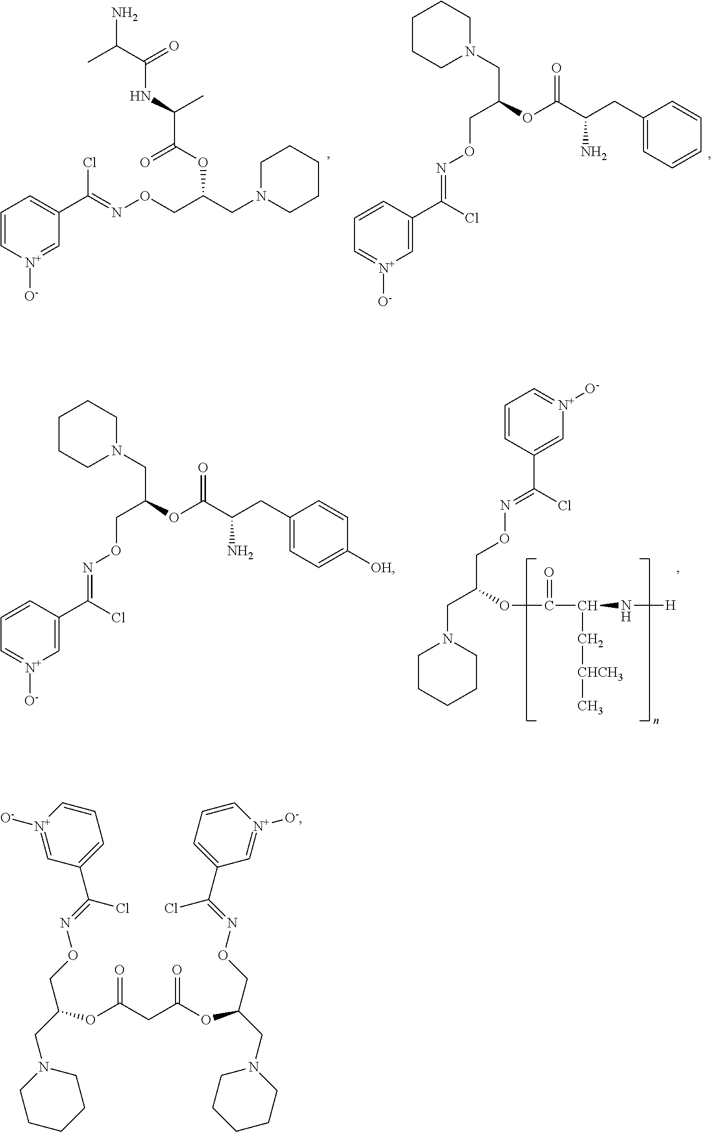

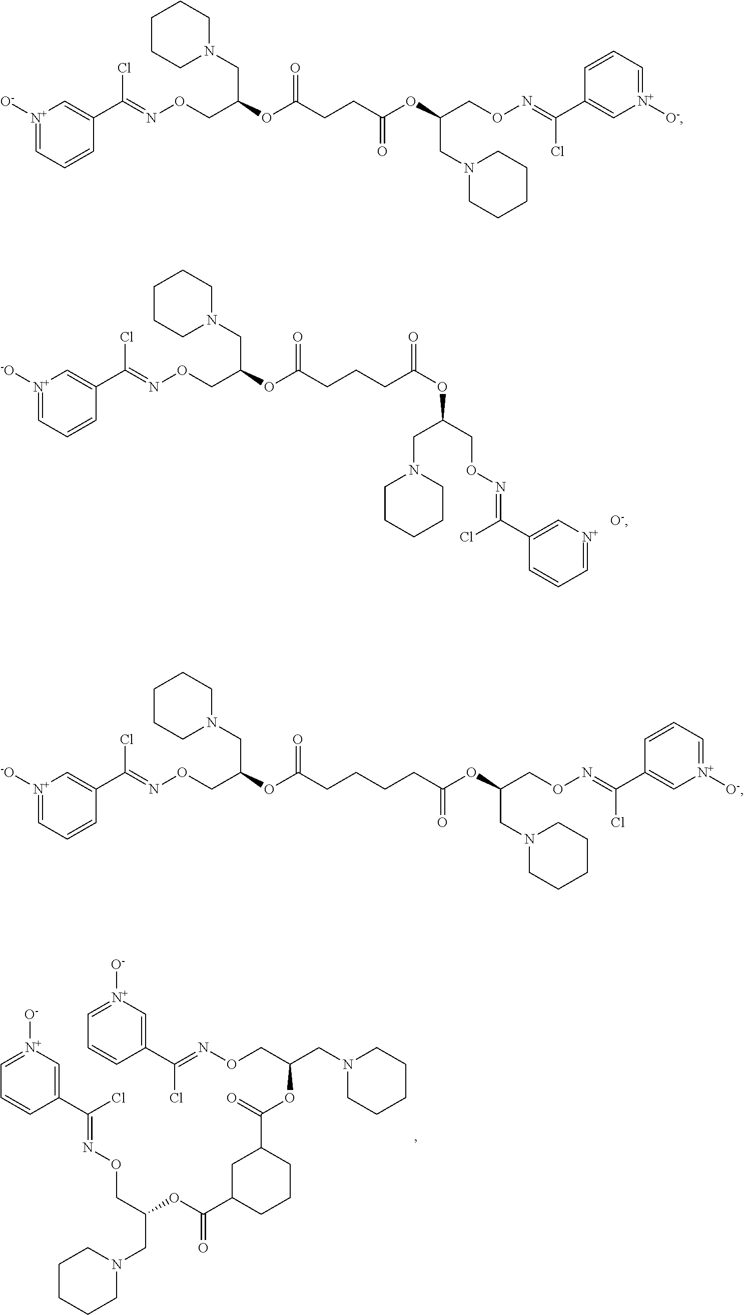

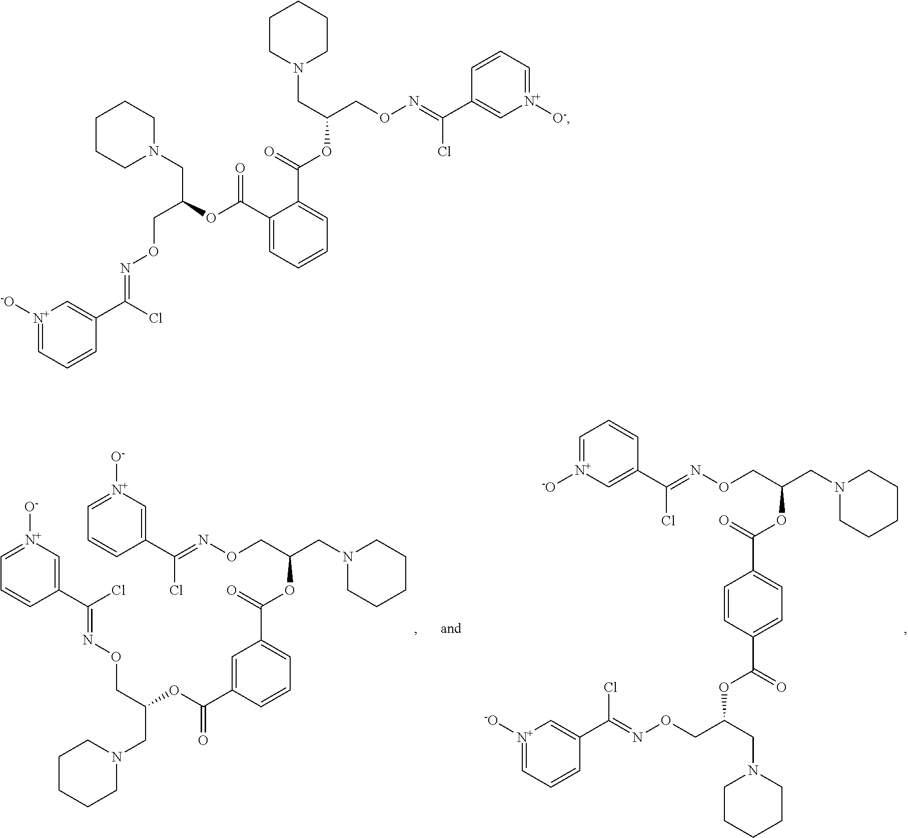

wherein n is 1-50;
or a pharmaceutically acceptable salt thereof.

In a further aspect, the present technology relates to an extended-release composition comprising a therapeutically effective amount of a compound of Formula IC, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In a still further aspect, the present technology relates to an extended-release composition comprising a therapeutically effective amount of a compound of Formula IC, wherein the compound of Formula IC is a compound having a structure selected from:

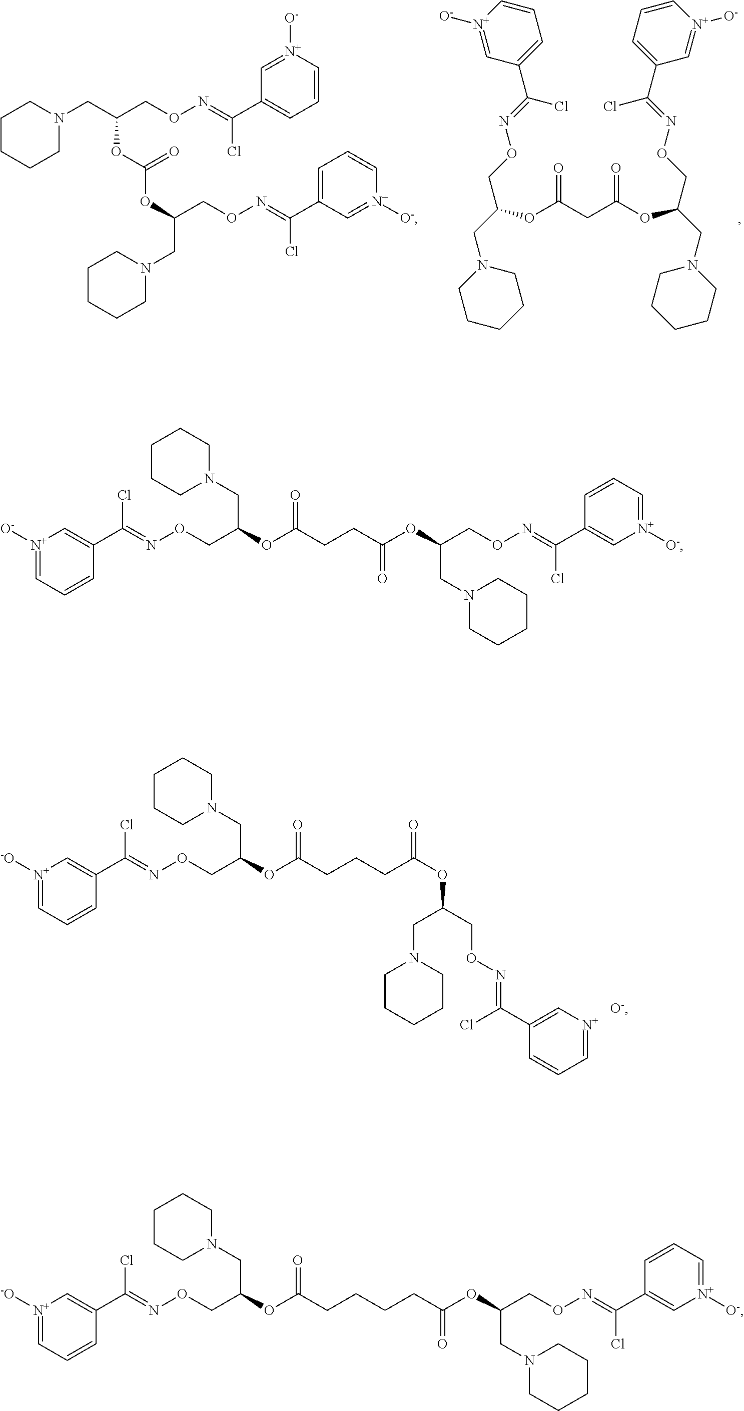

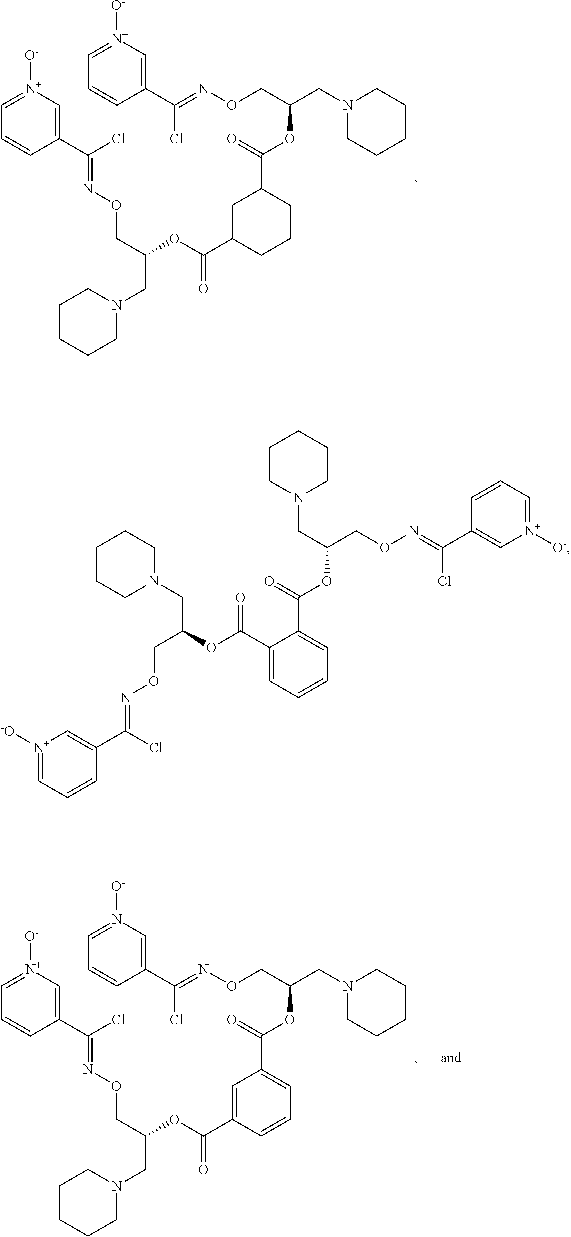

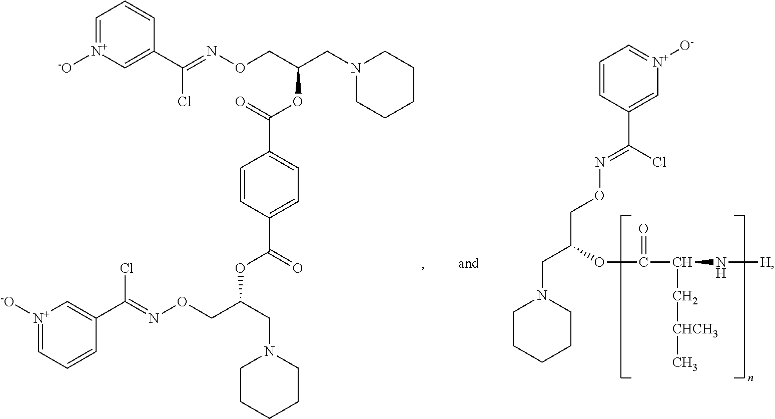

wherein n is 1 to 50;
or a pharmaceutically acceptable salt thereof.

In a further aspect, the present technology relates to an extended-release composition comprising a therapeutically effective amount of a compound of Formula I, wherein the compound of Formula I is a compound of Formula ID:

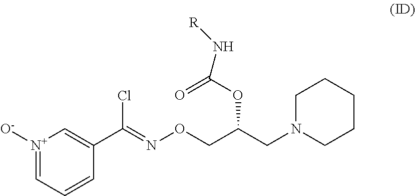

wherein R is selected from the group consisting of, alkenyl, alkenyl, aminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a still further aspect, the present technology relates to a composition comprising a therapeutically effective amount of a compound of Formula ID, wherein the compound of Formula ID is a compound having a structure selected from:

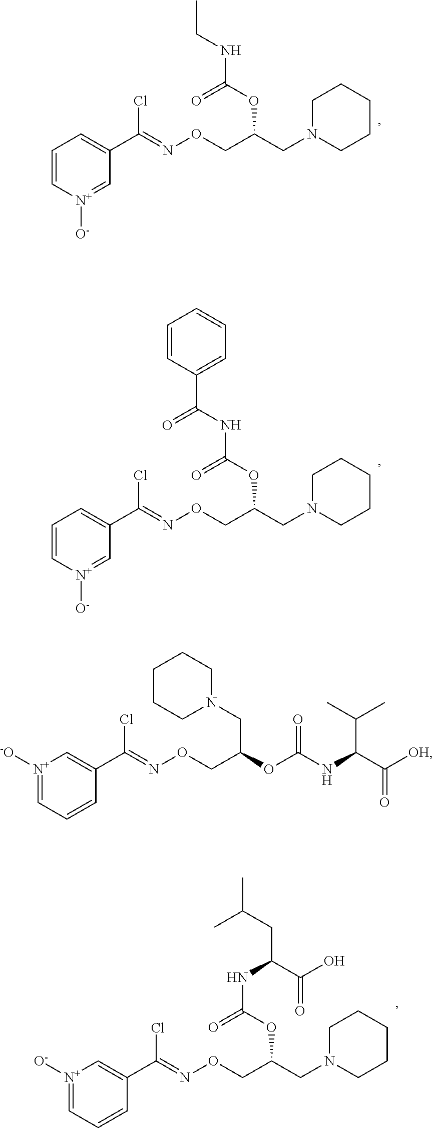

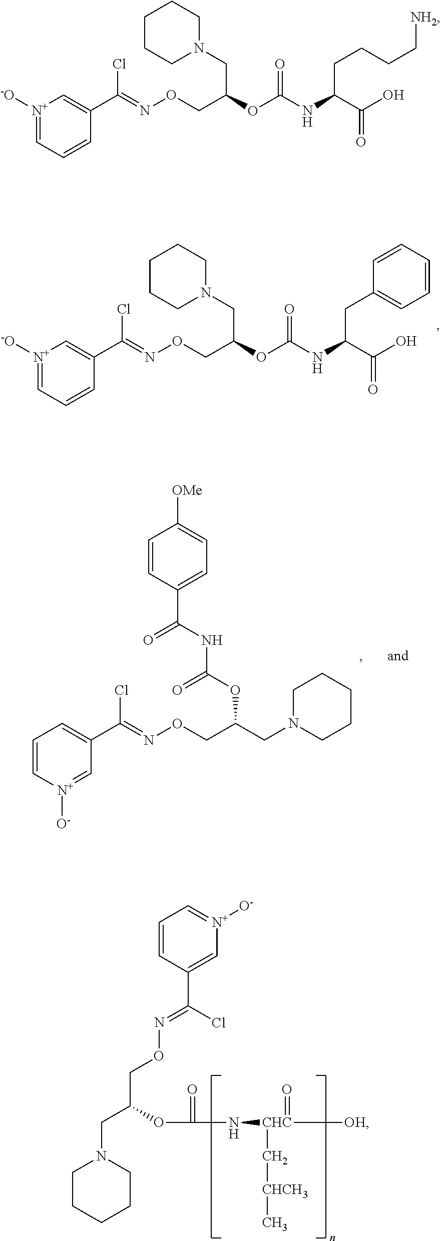

wherein n is 1-50;
or a pharmaceutically acceptable salt thereof.

In a further aspect, the present technology relates to an extended-release composition comprising a therapeutically effective amount of a compound of Formula ID, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In certain aspects, the present technology relates to an extended-release composition comprising:
a therapeutically effective amount of a compound of Formula II:

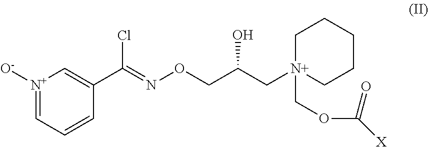

wherein X is selected from the group consisting of R, [O—R], [NR¹R²], and [NRH]
wherein O is oxygen, N is nitrogen, H is hydrogen, and
R, R¹ and R² are independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol
or a pharmaceutically acceptable salt thereof.

In a further aspect, the present technology relates to an extended-release composition comprising therapeutically effective amount of a compound of Formula II, wherein the compound of Formula II is selected from:

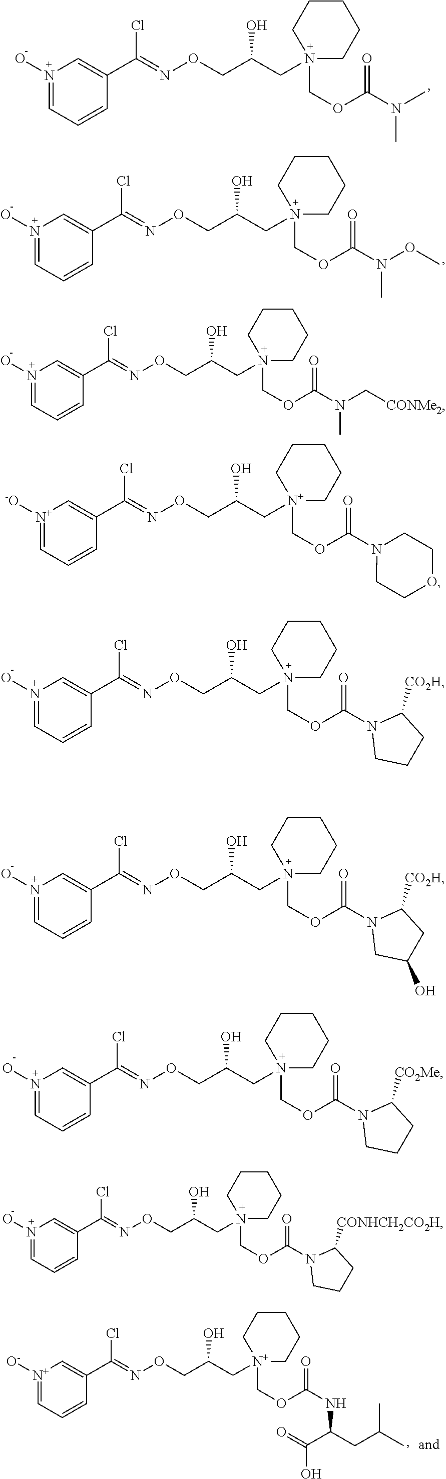

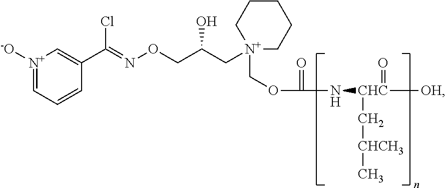

wherein n is 1-50;
or a pharmaceutically acceptable salt thereof.

In a further aspect, the present technology relates to an extended-release composition comprising a therapeutically effective amount of a compound of Formula II, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In certain aspect, the present technology relates to an extended-release composition compositing comprising a therapeutically effective amount of a compound selected from:

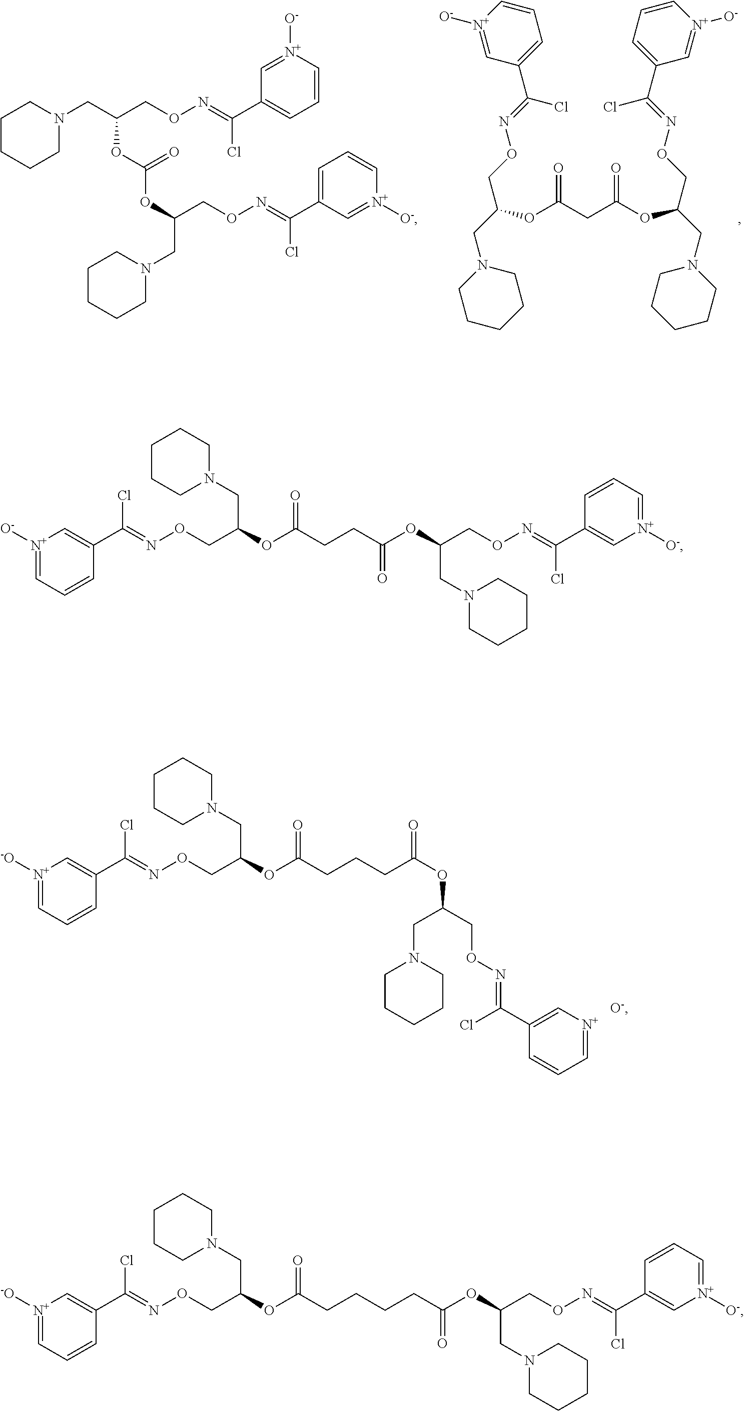

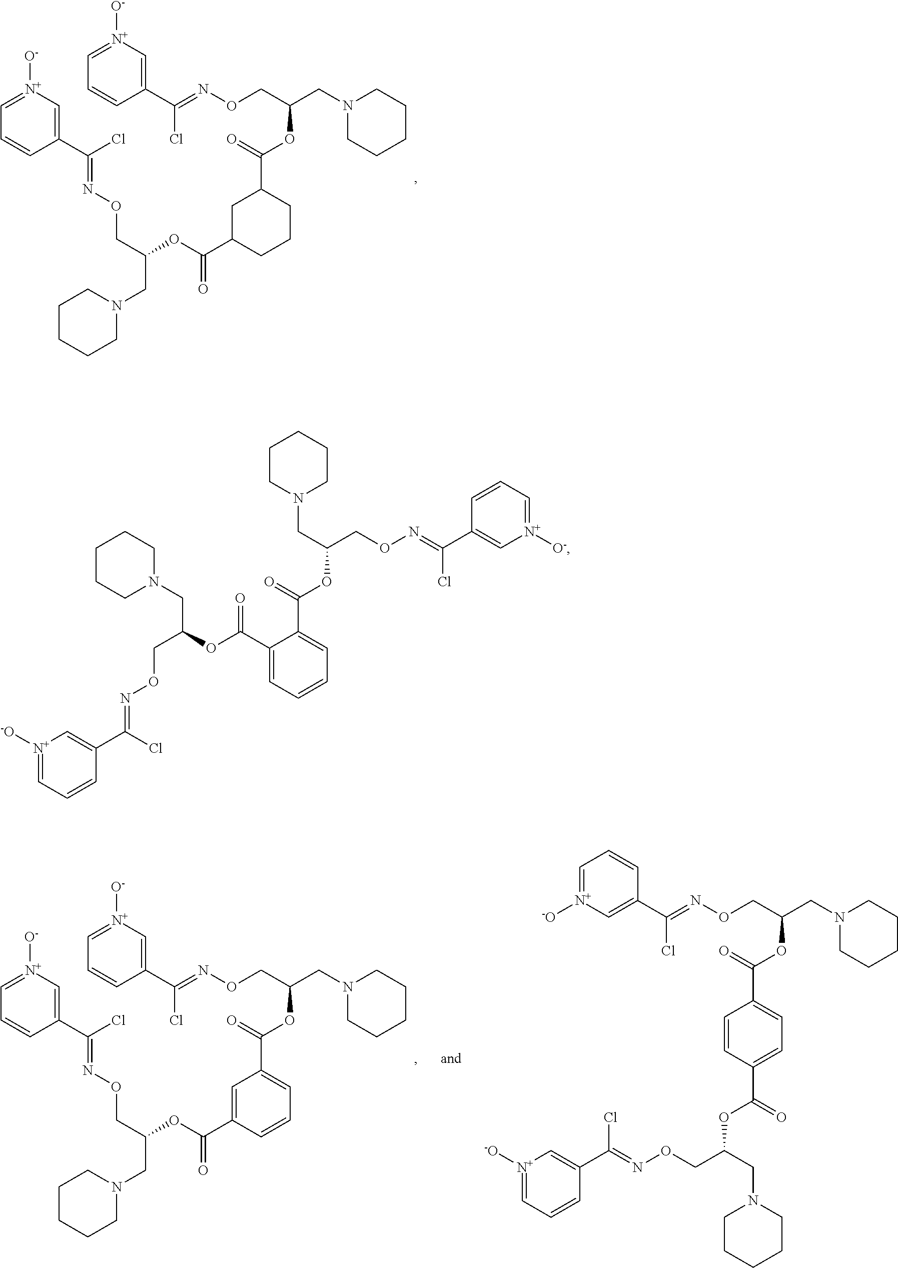

or a pharmaceutically acceptable salt thereof.

In certain aspects, the present technology relates to an extended-release composition comprising a therapeutically effective amount of a compound selected from

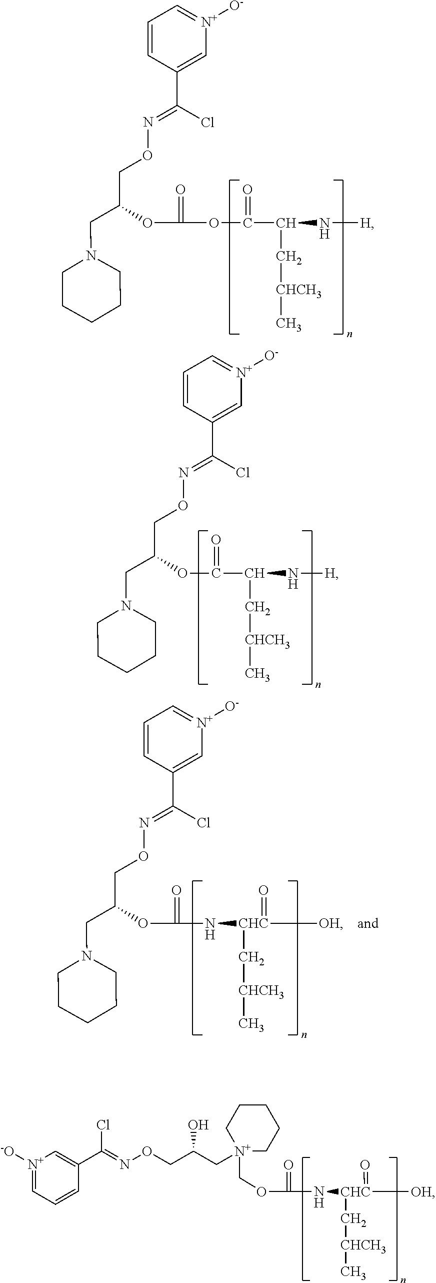

wherein n is 1-50; or a pharmaceutically acceptable salt thereof. Alternatively, n is 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In certain aspects, the present technology relates to an extended-release composition comprising a therapeutically effective amount of a compound of Formula III:

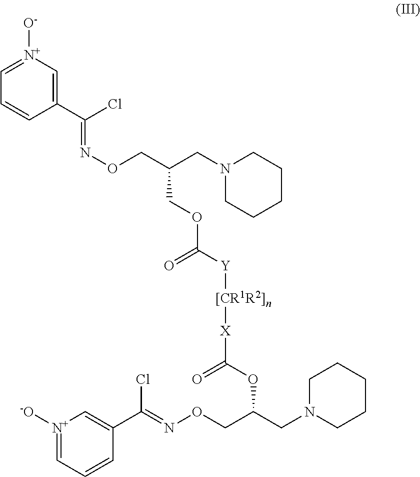

wherein X is selected from the group consisting of O, N, or absent,
Y is selected from the group consisting of O, N, or absent,
n is 1-10,
R¹ and R² are selected independently from each other and for each CR¹R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a further aspect, the present technology relates to an extended-release composition comprising a therapeutically effective amount of a compound of Formula IV, wherein n is alternatively 1-3, alternatively 3-5, alternatively 5-7, alternatively 7-9, alternatively 1-5, alternatively 5-10, alternatively 8-10, alternatively 3-8.

In certain aspects, the present technology relates to an extended-release composition comprising a therapeutically effective amount of a compound of Formula IV:

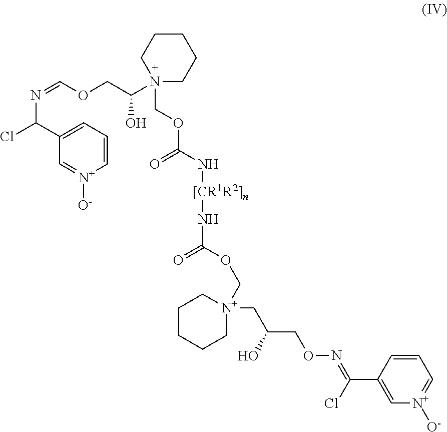

n is 1-10,
R¹ and R² are selected independently from each other and for each CR₁R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a further aspect, the present technology relates to an extended-release composition comprising a therapeutically effective amount of s compound of Formula IV, wherein n is alternatively 1-3, alternatively 3-5, alternatively 5-7, alternatively 7-9, alternatively 1-5, alternatively 5-10, alternatively 8-10, alternatively 3-8.

In certain aspects, the present technology relates to an extended-release composition comprising a therapeutically effective amount of a compound of Formula V

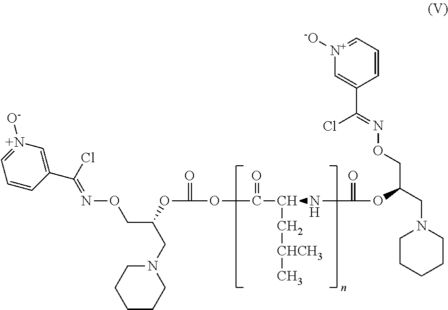

wherein n is 1-50,
or a pharmaceutically acceptable salt thereof.

In a further aspect, the present technology relates to an extended-release composition comprising a therapeutically effective amount of a compound of Formula V, wherein n is alternatively 1-3, alternatively 3-5, alternatively 5-7, alternatively 7-9, alternatively 1-5, alternatively 5-10, alternatively 8-10, alternatively 3-8.

In certain aspects, the present technology relates to an extended-release composition comprising a therapeutically effective amount of a compound of Formula VI:

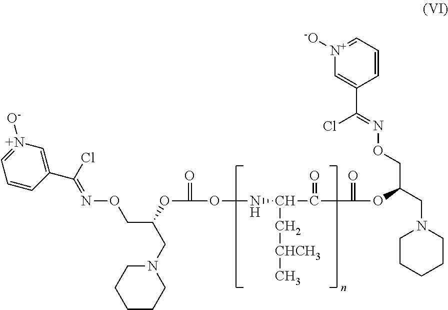

wherein n is 1-50;
or a pharmaceutically acceptable salt thereof.

In a further aspect, the present technology relates to an extended-release composition comprising a therapeutically effective amount of a compound of Formula VI, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In certain aspects, the present technology relates to an extended-release composition comprising a therapeutically effective amount of a compound of Formula VII:

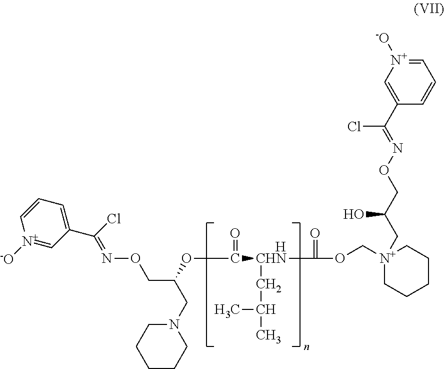

wherein n is 1-50, or a pharmaceutically acceptable salt thereof.

In a further aspect, the present technology relates to an extended-release composition comprising a therapeutically effective amount of a compound of Formula VII, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In a still further aspect, the present technology relates to an extended-release composition comprising a therapeutically effective amount of a compound of Formula I to Formula VII, wherein the pharmaceutically acceptable salt is selected from: sulfate, citrate, acetate, oxalate, chloride, hydrochloride, bromide, hydrobromide, iodide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

In a further aspect, the present technology relates to an extended-release composition comprising a therapeutically effective amount of a compound of Formula I to Formula VII, wherein the compound of Formula I to Formula VII is present as a stereoisomer of said compound. wherein the compound of Formula I to Formula VII is present as a stereoisomer and comprises a sub-structure selected from (+)-(R)—N-[2-hydroxy-3-(1-piperidinyl)-propoxy]-pyridine-1-oxide-3-carboximidoyl chloride, and (−)-(S)—N-[2-hydroxy-3-(1-piperidinyl)-propoxy]-pyridine-1-oxide-3-carboximidoyl chloride, (Z)—(R)—N-[2-hydroxy-3-(1-piperidinyl)-propoxy]-pyridine-1-oxide-3-carboximidoyl chloride, (E)-(R)—N-[2-hydroxy-3-(1 piperidinyl)-propoxy]-pyridine-1-oxide-3-carboximidoyl chloride, (Z)—(S)—N-[2-hydroxy-3-(1-piperidinyl)-propoxy]-pyridine-1-oxide-3-carboximidoyl chloride; and (E)-(S)—N-[2-hydroxy-3-(1-piperidinyl)-propoxy]-pyridine-1-oxide-3-carboximidoyl chloride.

In a further aspect, the present technology relates to an extended-release composition comprising an effective amount of a compound of Formula I or Formula II, wherein the compound of Formula I or the compound of Formula II is present as a racemate.

In certain aspects, the present technology relates to an extended-release composition comprising a therapeutically effective amount of a compound of Formula I to Formula VII, wherein the compound of Formula I to Formula VII is present in a form selected from polymorphs, crystals, and combinations thereof.

In certain aspects, the present technology relates to an extended-release composition comprising a therapeutically effective amount of a compound of Formula I to Formula VII, wherein the composition further comprises at least one excipient. In a still further aspect, the at least one excipient is selected from antiadherents, binders, coatings, disintegrants, gel forming agents, fillers, flavors and colors, glidants, lubricants, preservatives, sorbents and sweeteners. In a further aspect, the present technology relates to an extended-release composition comprising a therapeutically effective amount of a compound of Formula I or Formula II, wherein the at least one additional active pharmaceutical ingredient is selected from bimoclomol, miglustat, eliglustat, N-acetyl-L-leucine and 2-hydroxypropyl-β-cyclodextrin, and combinations thereof. In a further aspect, the at least one additional active pharmaceutical ingredient is selected from miglustat, N-acetyl-L-leucine, and combinations thereof. In a still further aspect, the at least one additional active pharmaceutical ingredient is a combination of miglustat and N-acetyl-L-leucine.

In a still further aspect, the present technology relates to an extended-release composition comprising a therapeutically effective amount of a compound of Formula I to Formula VII, wherein the composition is formulated for oral administration. In a still further aspect, the composition is a solid oral dosage formulation. In a still further aspect, the solid oral dosage formulation is selected from a sublingual, a gummy, a chewable tablet, a rapidly dissolving tablet, a tablet, a capsule, a caplet, a troche, a lozenge, an oral powder, a solution, a thin strip, an oral thin film (OTF), an oral strip, and a rectal film.

In a further aspect, the present technology relates to an extended-release composition comprising a therapeutically effective amount of a compound of Formula I to Formula VII, wherein the composition further comprises a release controlling agent selected from polymer coating, polymer matrix, and combinations thereof. In a still further aspect, the release controlling agent is a polymer coating selected from polyethylene oxides (PEO), hydroxypropyl cellulose (HPC), methylcellulose (MC), hydroxypropyl methylcellulose (HPMC), hydroxyethyl cellulose (HEC), ethylcellulose (EC), and sodium carboxymethyl cellulose (CMC). In a still further aspect, the release controlling agent is a polymer matrix selected from hydroxypropylmethylcellulose (HPMC), ethylcellulose (EC), methylcellulose, hypromellose acetate succinate, hypromellose phthalate, cellulose acetate, glycerin monostearate, glyceryl monooleate, glyceryl palmitate, glyceryl behenate, hydrogenated vegetable oil, guar gum, polyvinyl alcohol, alginates, xanthan gum, carnauba wax, yellow wax, white wax, zein, carrageenan, carbomers and agar In a still further aspect, the release controlling agent is a combination of a polymer coating and a polymer matrix.

In a further aspect, the present technology relates to an extended-release composition comprising a therapeutically effective amount of a compound of Formula I to Formula VII, wherein the composition is a liquid oral dosage formulation. In a still further aspect, the liquid oral dosage formulation is selected from syrups, emulsions, solutions, and suspensions.

In a still further aspect, the present technology relates to an extended-release composition comprising a therapeutically effective amount of a compound of Formula I to Formula VII, wherein the therapeutically effective amount of the compound of Formula I to Formula VII ranges from about 1 to about 5000 mg per day.

In a further aspect, the present technology relates to an extended-release composition comprising a therapeutically effective amount of a compound of Formula I to Formula VII, wherein the therapeutically effective amount of the compound of Formula I is administered as a unit dose. In certain aspects, the therapeutically effective amount of the compound of Formula I to Formula VII is administered in 1-2 unit doses. In a still further aspect, the therapeutically effective amount of the compound of Formula I to Formula VII is administered in 1 unit dose.

In certain aspects, the present technology relates to an extended-release composition comprising a therapeutically effective amount of a compound of Formula I to Formula VII; wherein the extended-release composition further comprises at least one additional active pharmaceutical ingredient and the at least one additional active pharmaceutical ingredient is designed to be immediately released upon administration. In a further aspect, the at least one additional active pharmaceutical ingredient is selected from arimoclomol, bimoclomol, miglustat, eligustat, and N-acetyl-L-leucine.

In certain aspects, the present technology relates to a method of treating a neurodegenerative or metabolic disease or disorder in a patient in need thereof, the method comprising:
administering to the patient in need thereof a composition comprising a therapeutically effective amount of a compound having a structure of Formula I:

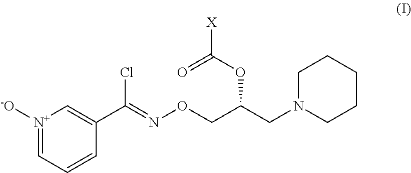

wherein X is selected from the group consisting of R, [O—R], [NR¹R²], and [NRH]
wherein O is oxygen, N is nitrogen, H is hydrogen, and
R, R¹ and R² are independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol,
or a pharmaceutically acceptable salt thereof.

In a further aspect, the present technology relates to a method of treating a neurodegenerative or metabolic disease or disorder, the method comprising administering to the patient in need thereof a composition comprising a therapeutically effective amount of the compound of Formula I, wherein the compound is a compound having a structure of Formula IA:

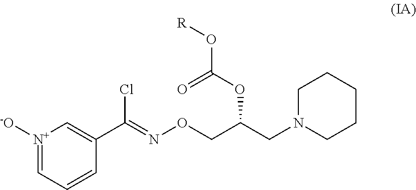

wherein R is selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol; or a pharmaceutically acceptable salt thereof.

In a still further aspect, the present technology relates to a method of treating a neurodegenerative or metabolic disease or disorder, the method comprising administering to the patient in need thereof a therapeutically effective amount of a compound of Formula IA, wherein the compound of Formula IA is a compound having a structure selected from:

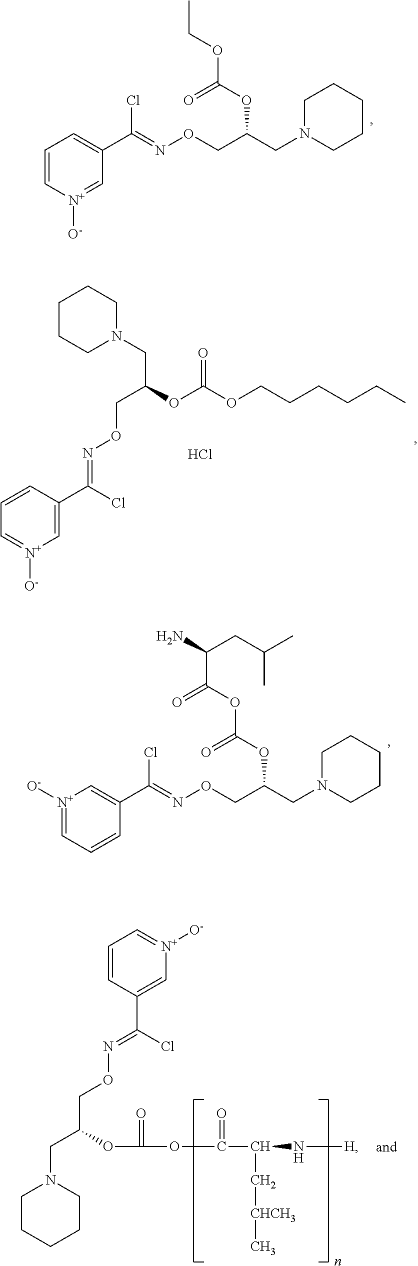

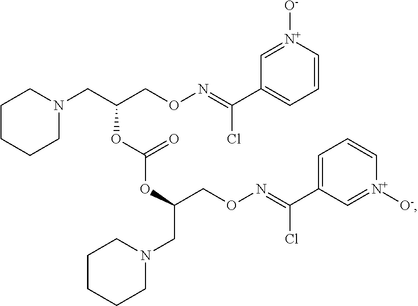

wherein n is 1-50;
or a pharmaceutically acceptable salt thereof.

In a further aspect, the present technology relates to a method of treating a neurodegenerative or metabolic disease or disorder, wherein the method comprises administering to the patient in need thereof a composition comprising a compound of Formula IA, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In a further aspect, the present technology relates to a method of treating a neurodegenerative or metabolic disease or disorder, wherein the method comprises administering to the patient in need thereof a composition comprising a compound of Formula I, wherein the compound of Formula I is a compound having a structure of Formula IB:

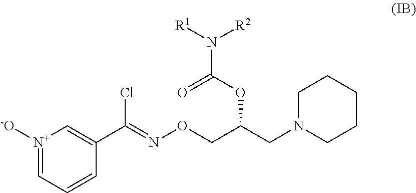

wherein R¹ and R² are independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol; or
or a pharmaceutically acceptable salt thereof.

In a further aspect, the present technology relates to a method of treating a neurodegenerative or metabolic disease or disorder, wherein the method comprises administering to the patient in need thereof a composition comprising a compound of Formula IB, wherein the compound of Formula IB is a compound having a structure selected from:

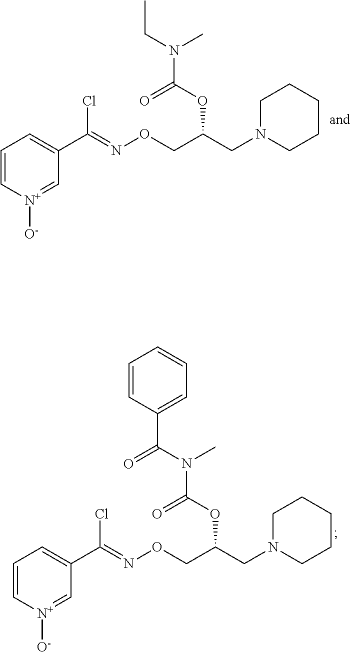 [SMILES: CCN(C)C(=O)O[C@@H](CO/N=C(\Cl)c1ccc[n+]([O-])c1)CN1CCCCC1 ; CN(C(=O)O[C@@H](CO/N=C(\Cl)c1ccc[n+]([O-])c1)CN1CCCCC1)C(=O)c1ccccc1]

or a pharmaceutically acceptable salt thereof.

In a further aspect, the present technology relates to a method of treating a neurodegenerative or metabolic disease or disorder, wherein the method comprises administering to the patient in need thereof a composition comprising a compound of Formula I, wherein the compound of Formula I is a compound having a structure of Formula IC:

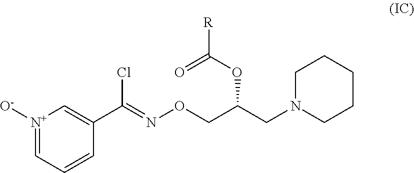

wherein R is selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a further aspect, the present technology relates to a method of treating a neurodegenerative or metabolic disease or disorder, wherein the method comprises administering to the patient in need thereof a composition comprising a compound of Formula IC, wherein the compound of Formula IC is a compound having a structure selected from:

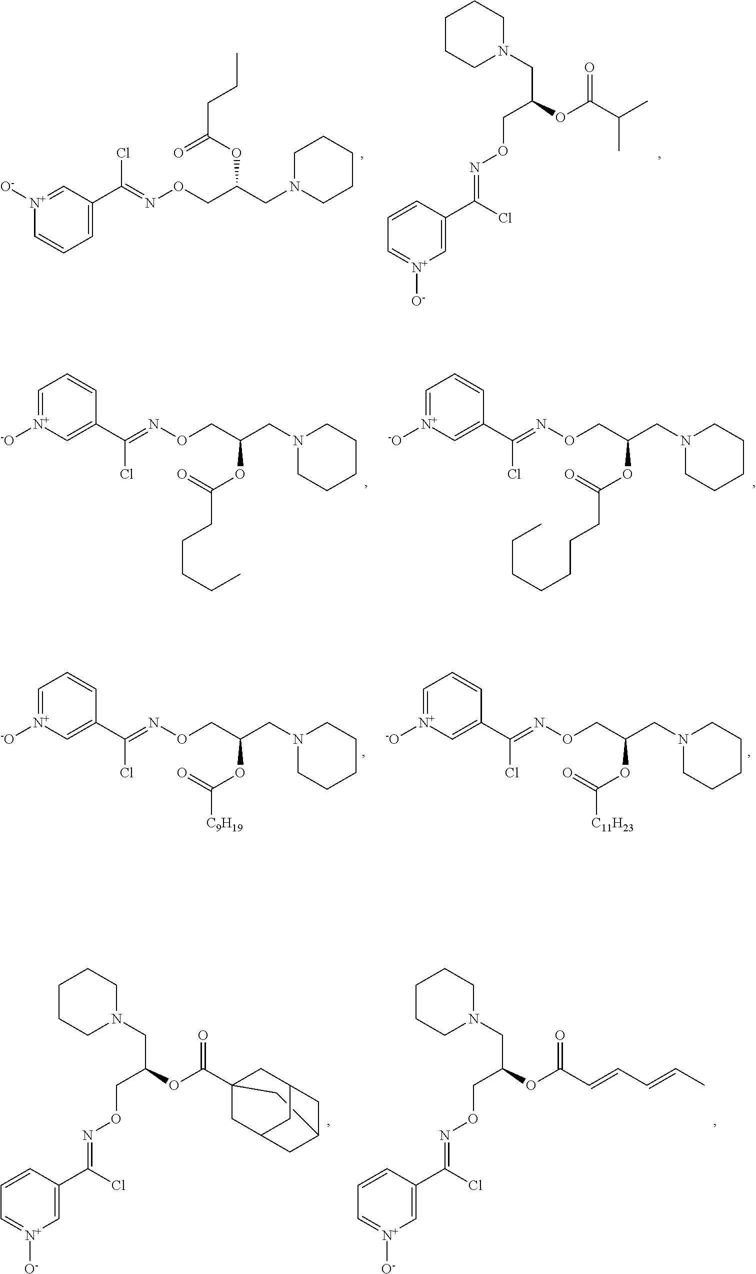

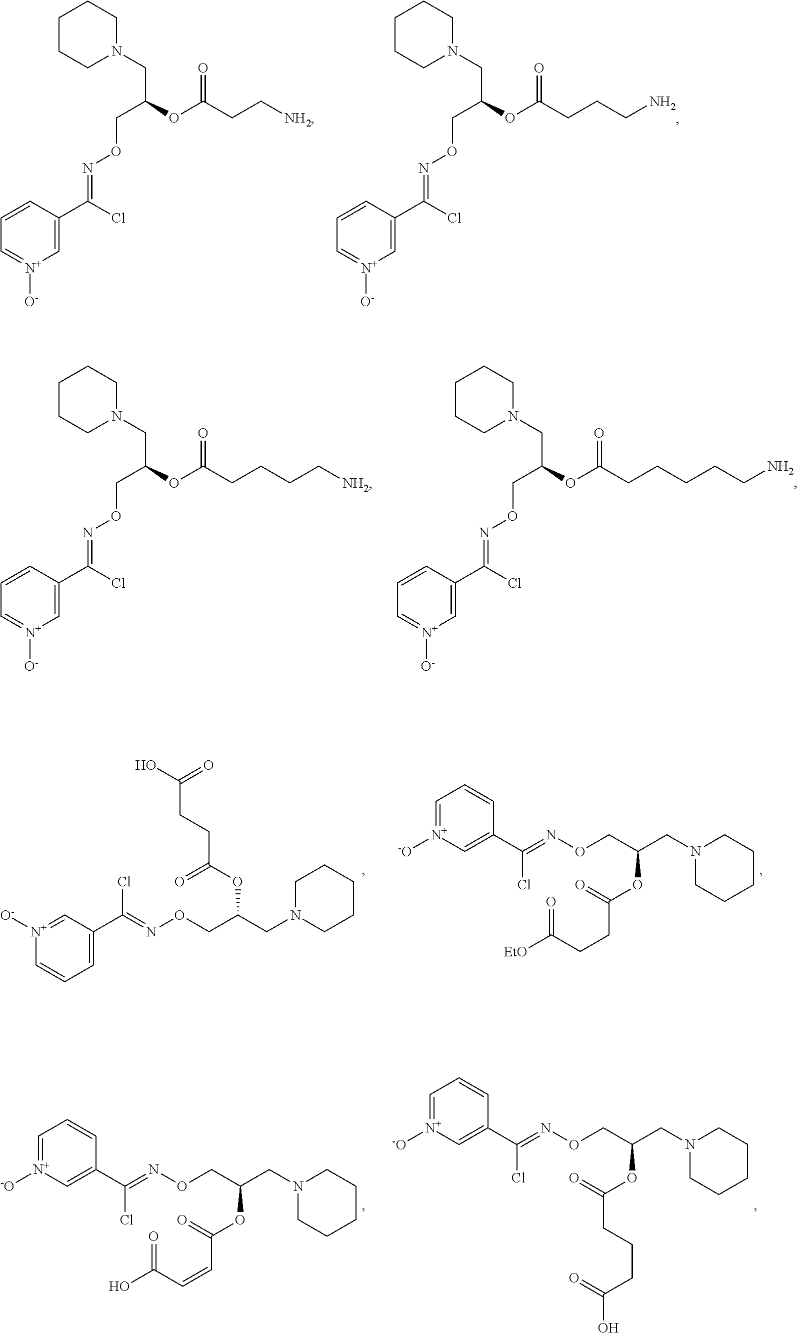

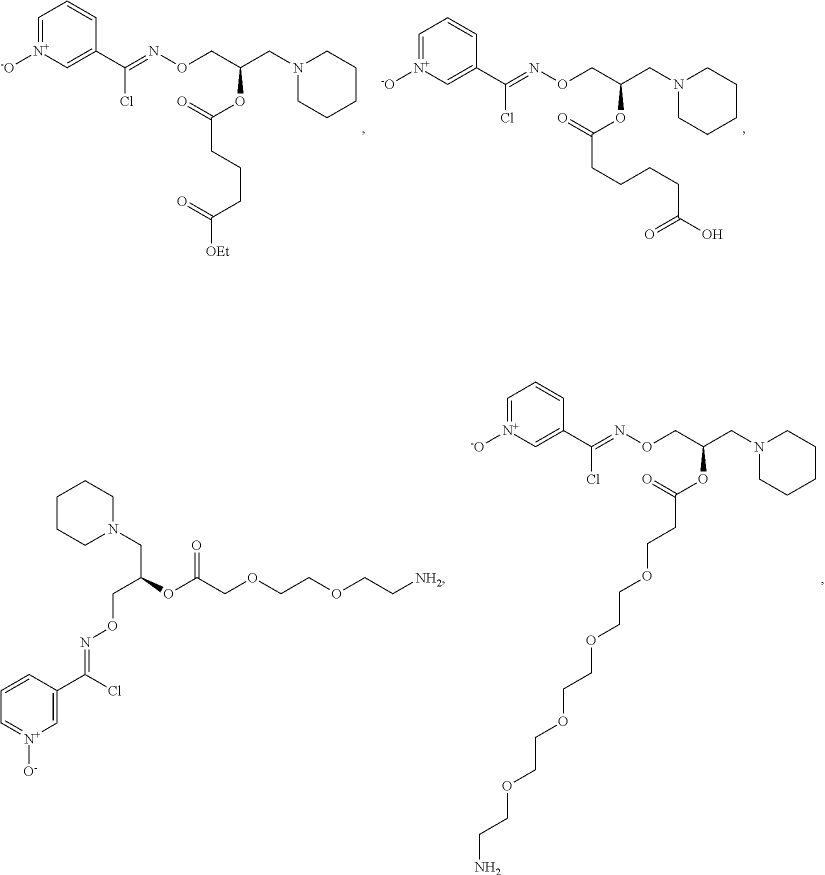

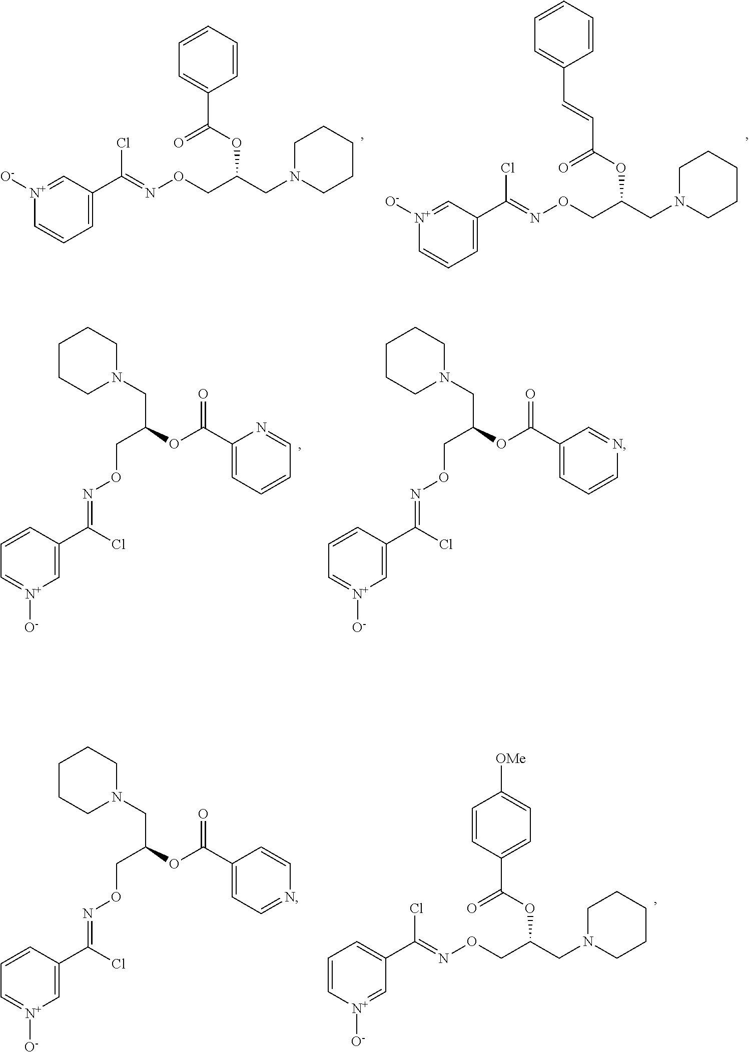

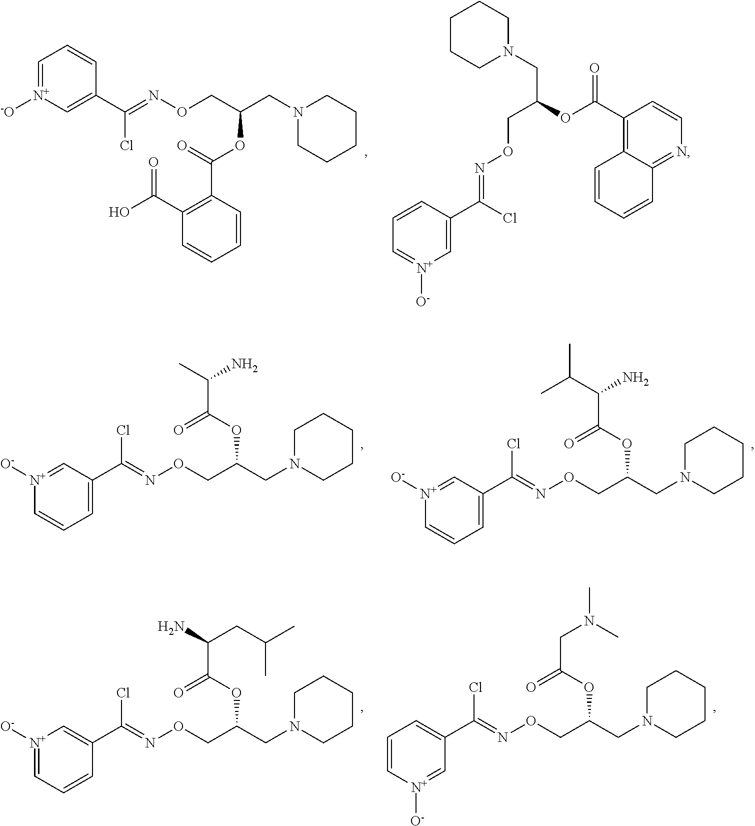

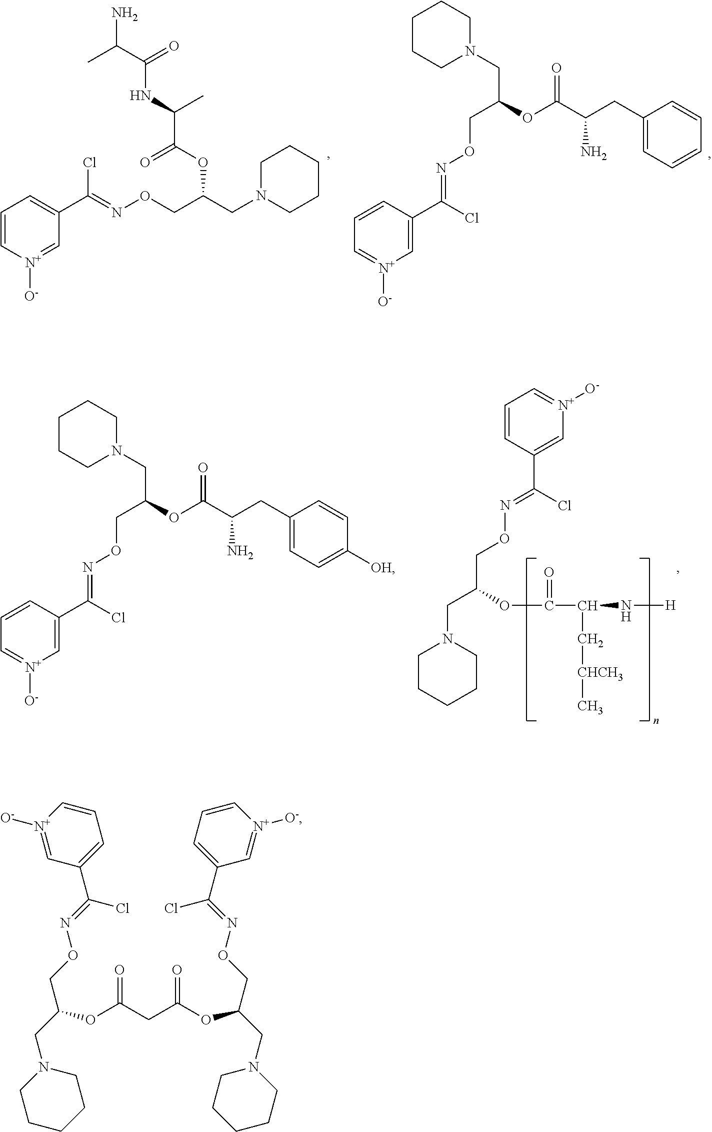

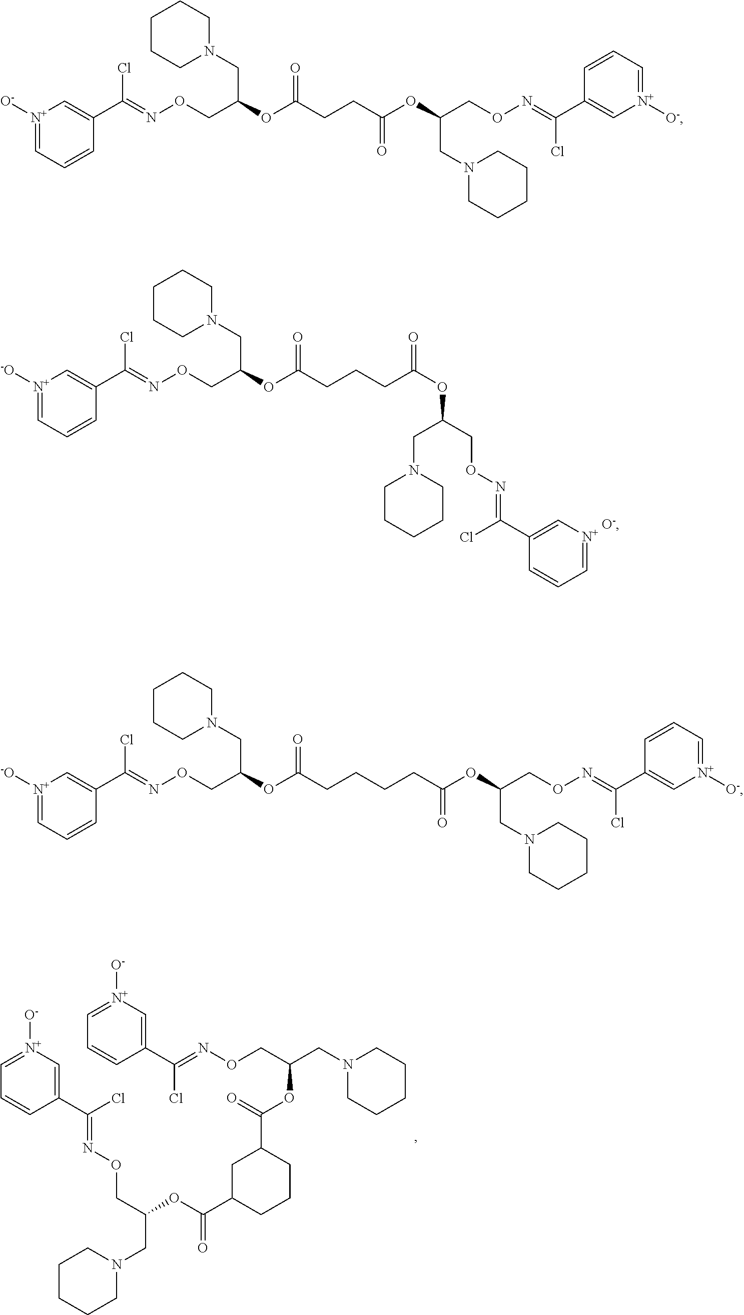

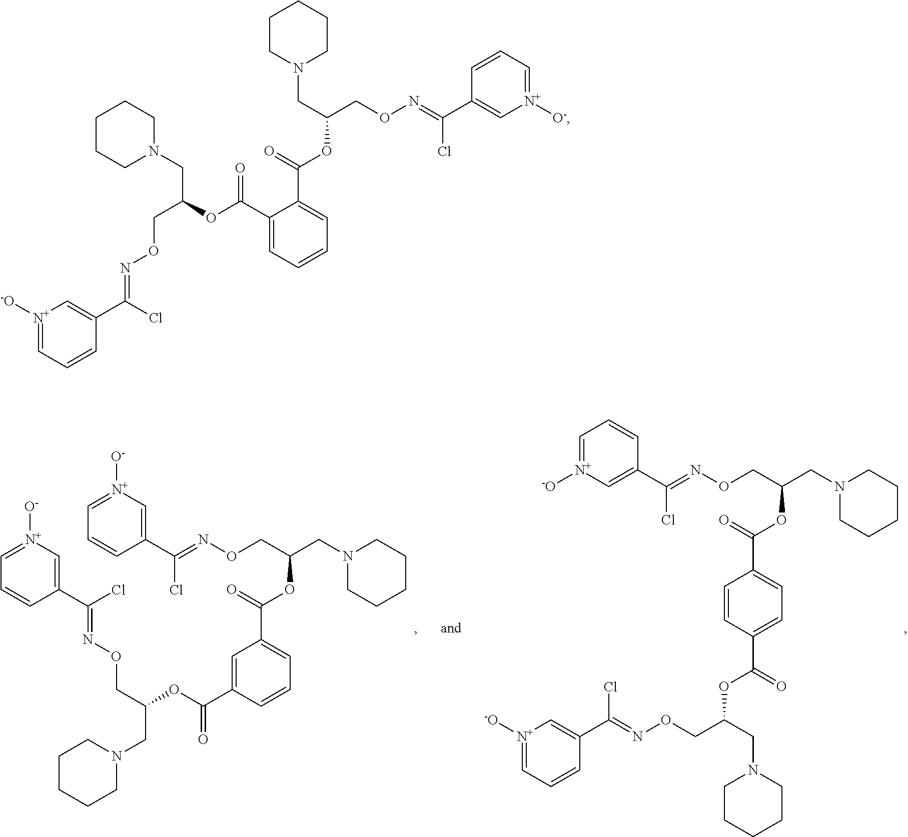

wherein n is 1-50;
or a pharmaceutically acceptable salt thereof.

In a further aspect, the present technology relates to a method of treating a neurodegenerative or metabolic disease or disorder, wherein the method comprises administering to the patient in need thereof a composition comprising a compound of Formula IC, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In a further aspect, the present technology relates to a method of treating a neurodegenerative or metabolic disease or disorder, wherein the method comprises administering to the patient in need thereof a composition comprising a compound of Formula IC, wherein the compound of Formula IC is a compound having a structure selected from:

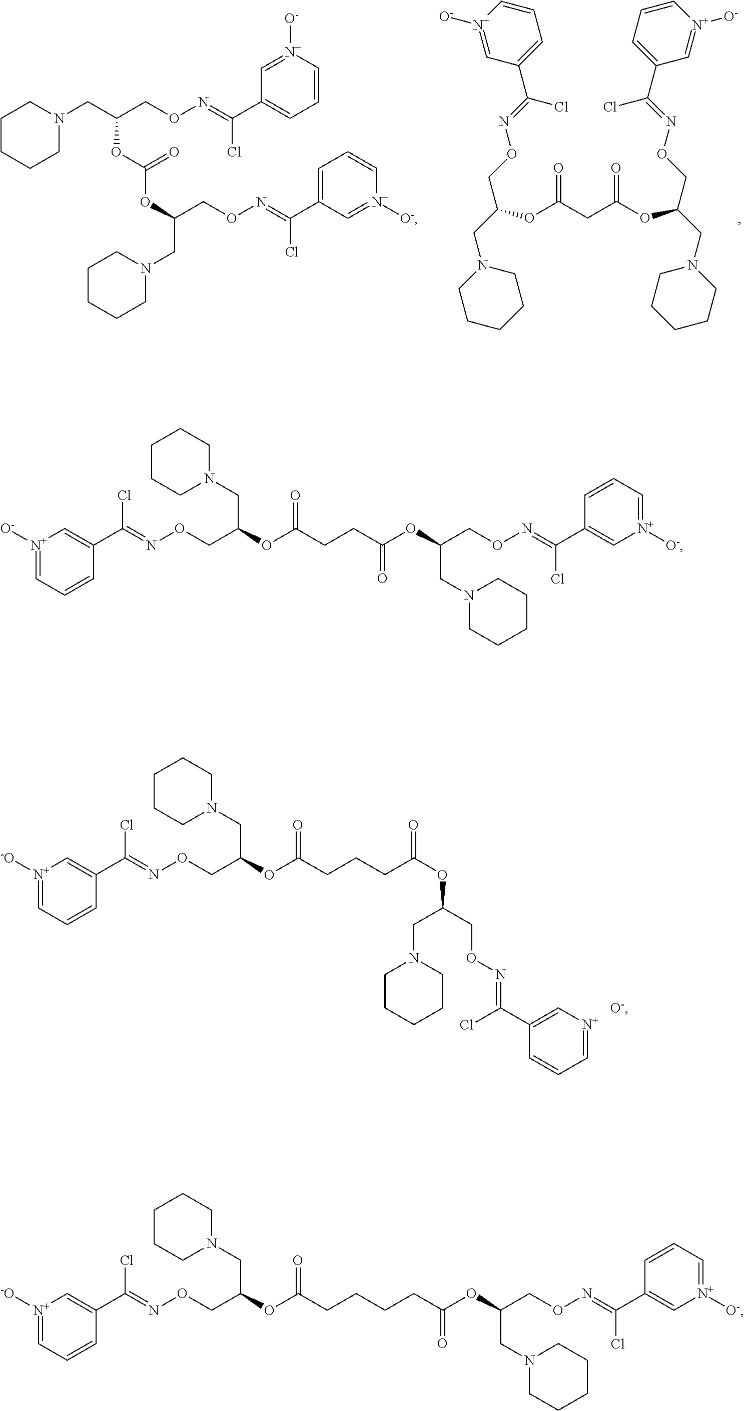

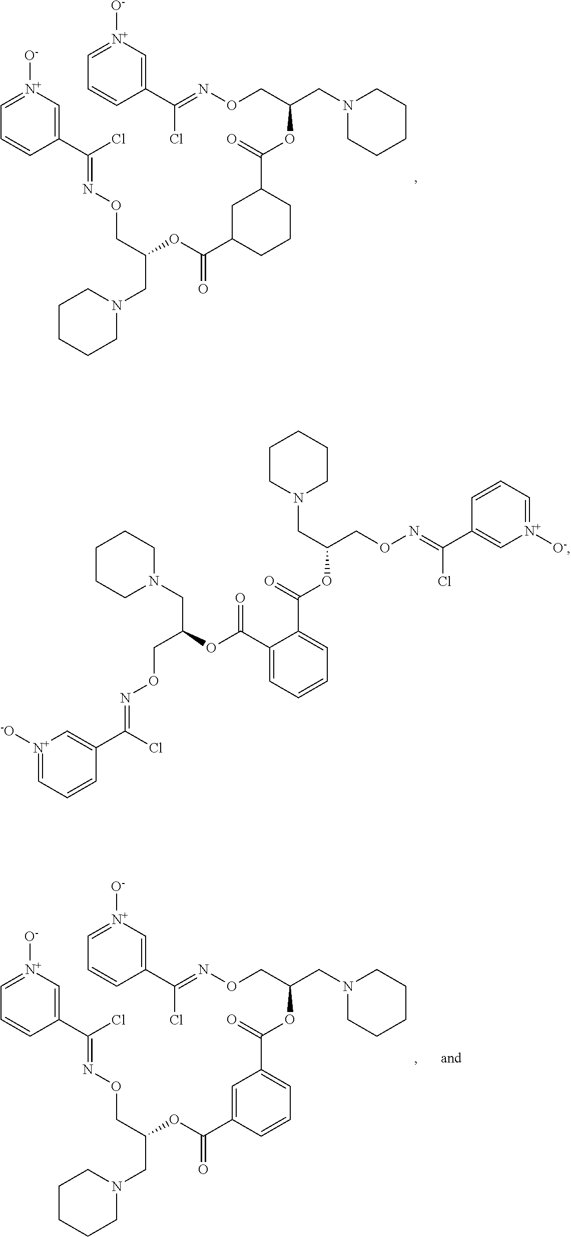

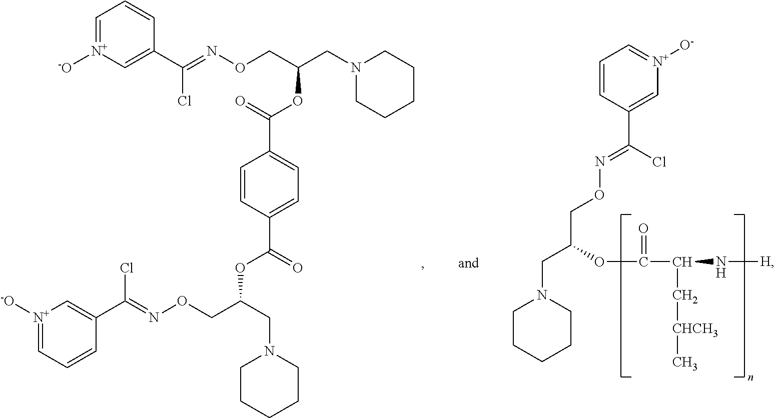

wherein n is 1-50;
or a pharmaceutically acceptable salt thereof.

In a further aspect, the present technology relates to a method of treating a neurodegenerative or metabolic disease or disorder, wherein the method comprises administering to the patient in need thereof a composition comprising a compound of Formula I, wherein the compound of Formula I is a compound having a structure of Formula ID:

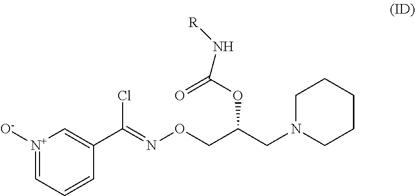

wherein R is selected from the group consisting of alkenyl, alkenyl, aminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a further aspect, the present technology relates to a method of treating a neurodegenerative or metabolic disease or disorder, wherein the method comprises administering to the patient in need thereof a composition comprising a compound of Formula ID, wherein the compound of Formula ID is a compound having a structure selected from:

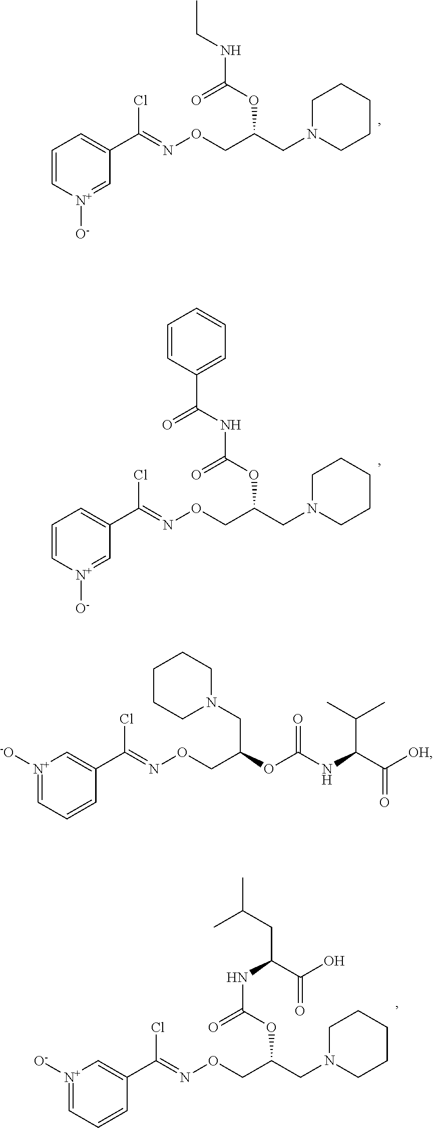

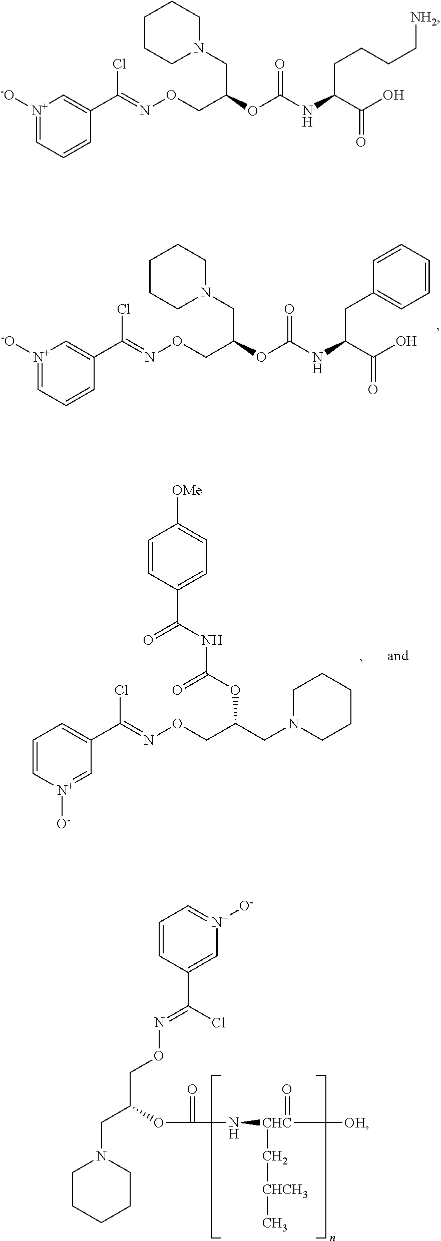

wherein n is 1-50;
or a pharmaceutically acceptable salt thereof.

In a further aspect, the present technology relates to a method of treating a neurodegenerative or metabolic disease or disorder, wherein the method comprises administering to the patient in need thereof a composition comprising a compound of Formula ID, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In certain aspects, the present technology relates to a method of treating a neurodegenerative or metabolic disease or disorder, the method comprising:
administering to the patient in need thereof a therapeutically effective amount of the compound of Formula II:

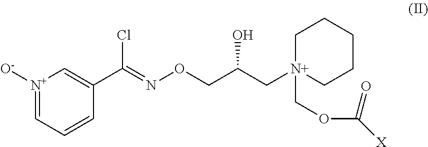

wherein R¹ and R² are independently selected from alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a further aspect, the present technology relates to a method of treating a neurodegenerative or metabolic disease or disorder, wherein the method comprises administering to the patient in need thereof a composition comprising a compound of Formula II, wherein the compound of Formula II has a structure selected from:

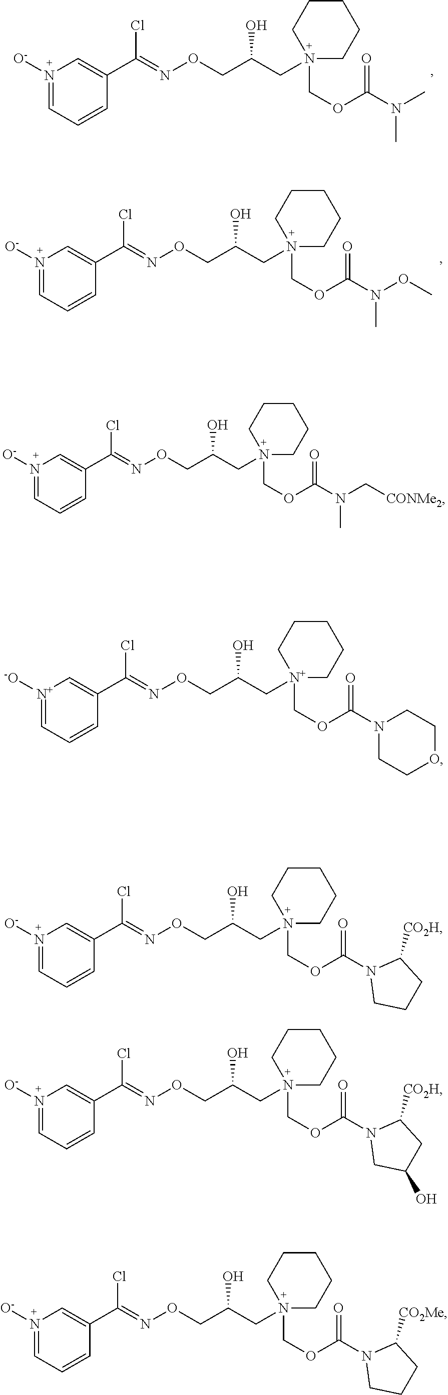

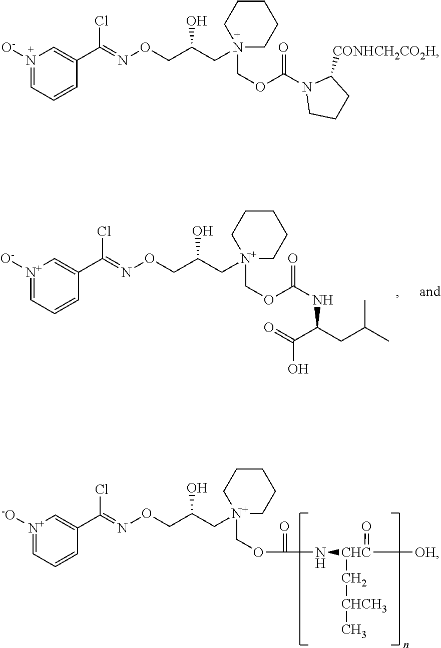

wherein n is 1-50;
or a pharmaceutically acceptable salt thereof.

In a further aspect, the present technology relates to a method of treating a neurodegenerative or metabolic disease or disorder, wherein the method comprises administering to the patient in need thereof a composition comprising a compound of Formula II, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In certain aspects, the present technology relates to a method of treating a neurodegenerative or metabolic disease or disorder, wherein the method comprises administering to the patient in need thereof a composition comprising therapeutically effective amount of a compound of Formula III:

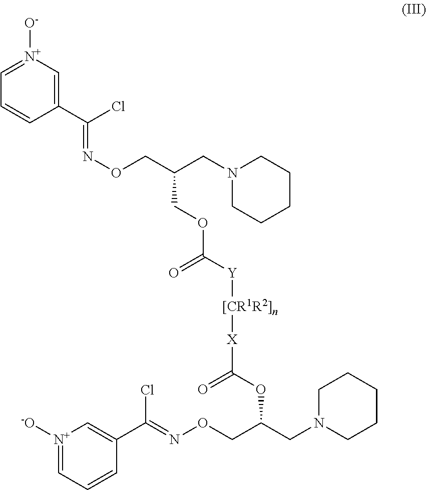

wherein X is selected from the group consisting of O, N, or absent,
Y is selected from the group consisting of O, N, or absent,
n is 1-10,
R¹ and R² are selected independently from each other and for each CR¹R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a further aspect, the present technology relates to a method of treating a neurodegenerative or metabolic disease or disorder, wherein the method comprises administering to the patient in need thereof a composition comprising therapeutically effective amount of a compound of Formula IV, wherein n is alternatively 1-3, alternatively 3-5, alternatively 5-7, alternatively 7-9, alternatively 1-5, alternatively 5-10, alternatively 8-10, alternatively 3-8.

In certain aspects, the present technology relates to a method of treating a neurodegenerative or metabolic disease or disorder, wherein the method comprises administering to the patient in need thereof a composition comprising therapeutically effective amount of a compound of Formula IV:

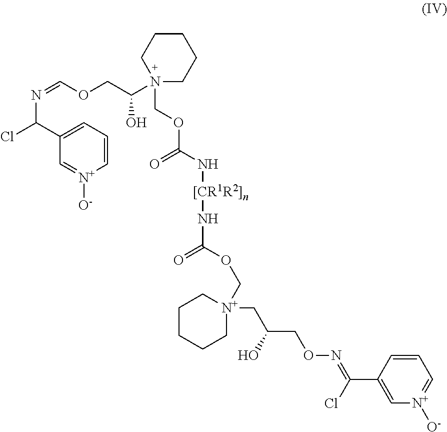

n is 1-10,
R¹ and R² are selected independently from each other and for each CR₁R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a further aspect, the present technology relates to a method of treating a neurodegenerative or metabolic disease or disorder, wherein the method comprises administering to the patient in need thereof a composition comprising therapeutically effective amount of a compound of Formula IV, wherein n is alternatively 1-3, alternatively 3-5, alternatively 5-7, alternatively 7-9, alternatively 1-5, alternatively 5-10, alternatively 8-10, alternatively 3-8.

In certain aspects, the present technology relates to a method of treating a neurodegenerative or metabolic disease or disorder, wherein the method comprises administering to the patient in need thereof a composition comprising therapeutically effective amount of a compound of Formula V

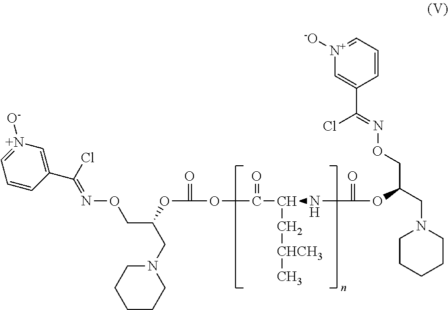

wherein n is 1-50,
or a pharmaceutically acceptable salt thereof.

In a further aspect, the present technology relates to a method of treating a neurodegenerative or metabolic disease or disorder, wherein the method comprises administering to the patient in need thereof a composition comprising therapeutically effective amount of a compound of Formula V, wherein n is alternatively 1-3, alternatively 3-5, alternatively 5-7, alternatively 7-9, alternatively 1-5, alternatively 5-10, alternatively 8-10, alternatively 3-8.

In certain aspects, the present technology relates to an extended-release composition comprising a therapeutically effective amount of a compound of Formula VI:

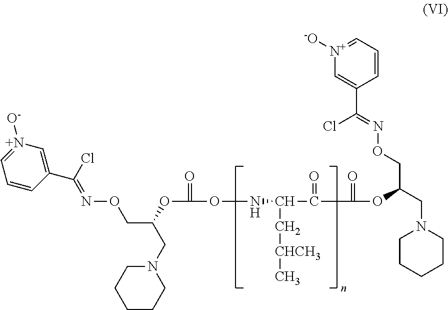

wherein n is 1-50;
or a pharmaceutically acceptable salt thereof.

In a further aspect, the present technology relates to a method of treating a neurodegenerative or metabolic disease or disorder, wherein the method comprises administering to the patient in need thereof a composition comprising therapeutically effective amount of a compound of Formula VI, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In certain aspects, the present technology relates to a method of treating a neurodegenerative or metabolic disease or disorder, wherein the method comprises administering to the patient in need thereof a composition comprising therapeutically effective amount of a compound of Formula VII:

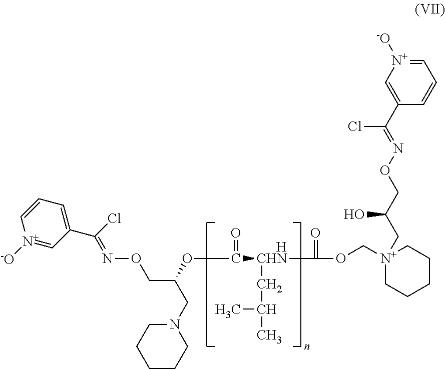

wherein n is 1-50, or a pharmaceutically acceptable salt thereof.

In a further aspect, the present technology relates to a method of treating a neurodegenerative or metabolic disease or disorder, wherein the method comprises administering to the patient in need thereof a composition comprising therapeutically effective amount of a compound of Formula VII, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In certain aspects, the present technology relates to a method of treating a neurodegenerative or metabolic disease or disorder, wherein the method comprises administering to the patient in need thereof a composition comprising therapeutically effective amount of a compound selected from:

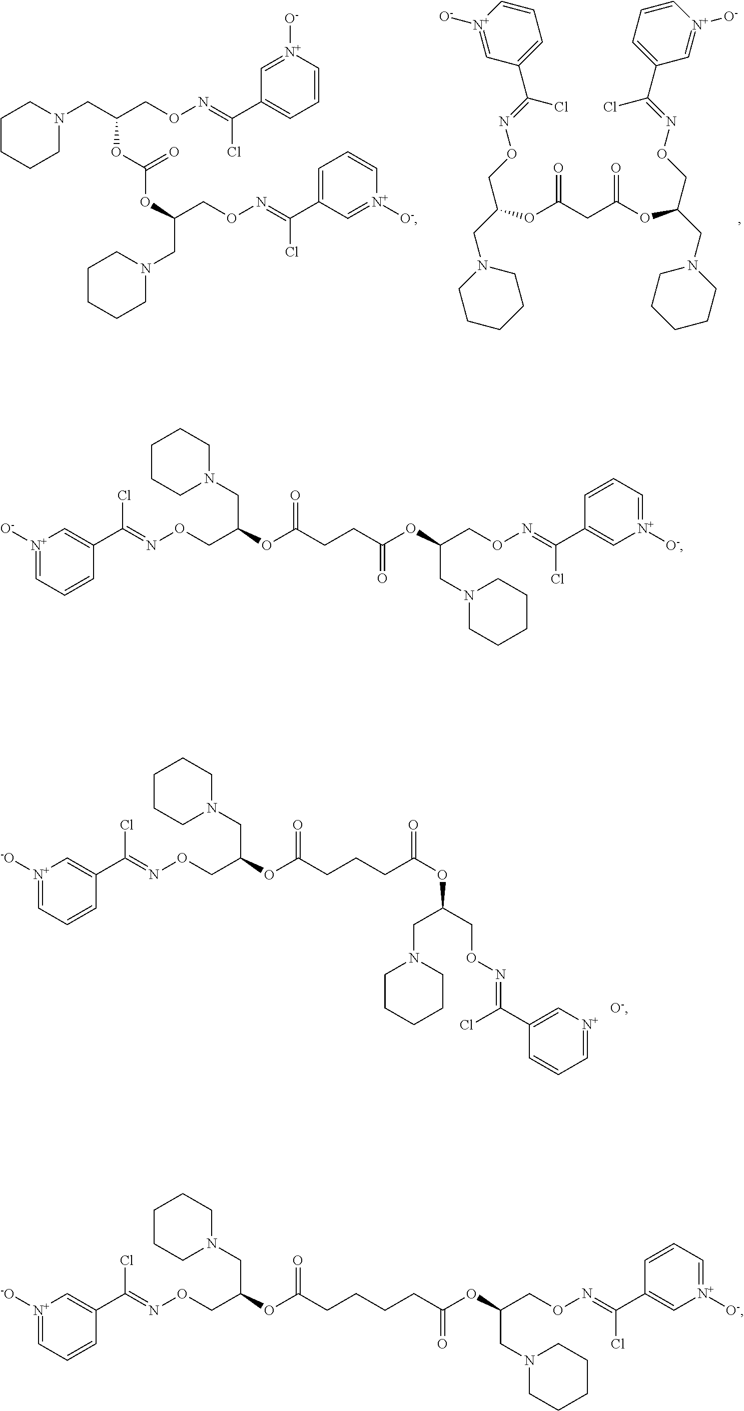

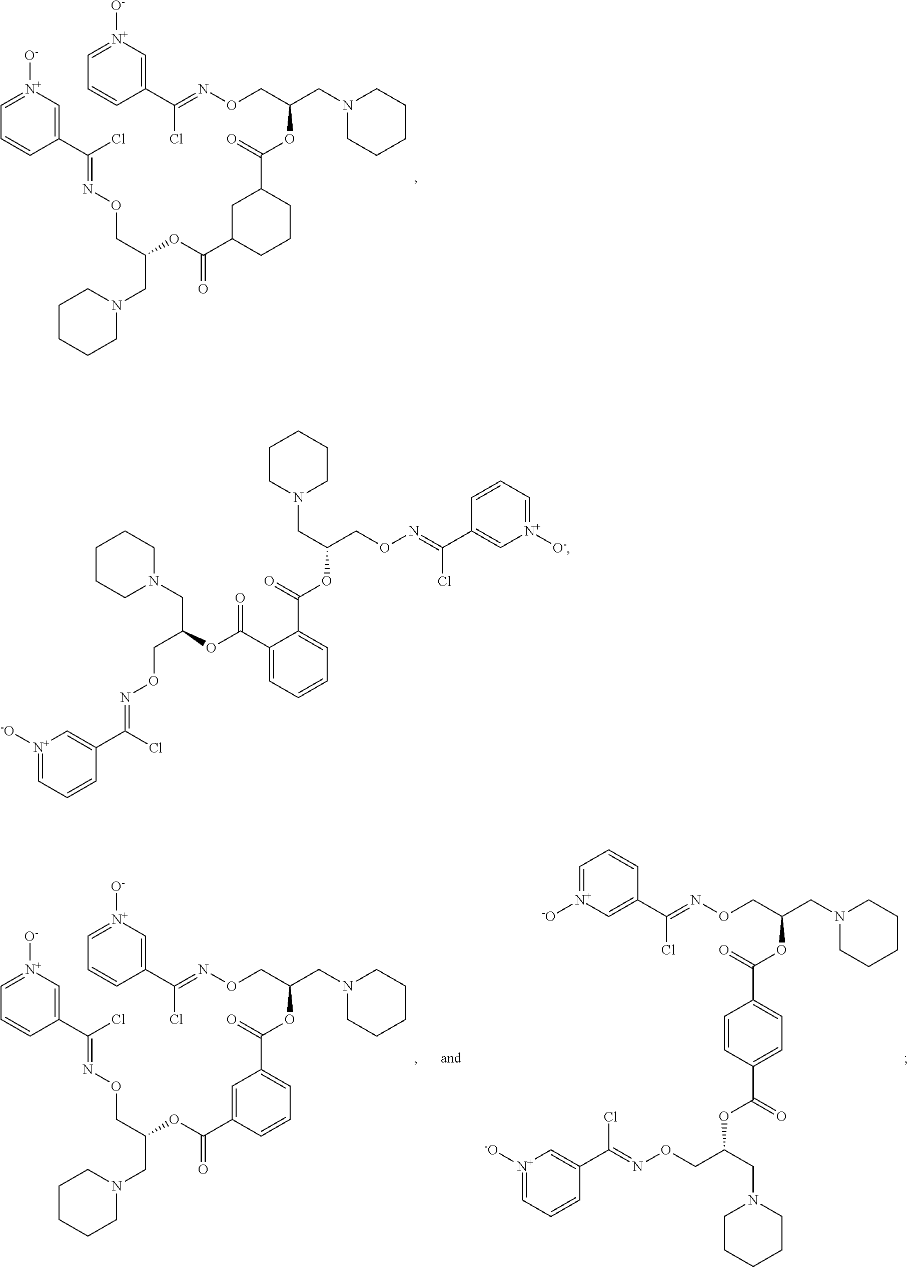

or a pharmaceutically acceptable salt thereof.

In certain aspects, the present technology relates to a method of treating a neurodegenerative or metabolic disease or disorder, wherein the method comprises administering to the patient in need thereof a composition comprising therapeutically effective amount of a compound selected from,

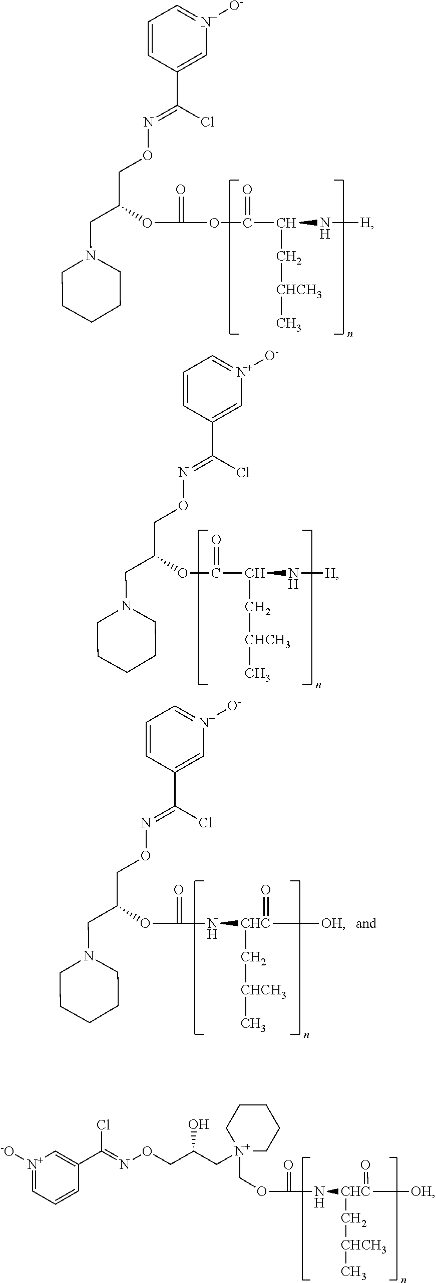

wherein n is 1-50; or a pharmaceutically acceptable salt thereof. Alternatively, n is 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In a further aspect, the present technology relates to a method of treating a neurodegenerative or metabolic disease or disorder, wherein the method comprises administering to the patient in need thereof the composition comprising the compound of Formula I to Formula VII, wherein the pharmaceutically acceptable salt is selected from: sulfate, citrate, acetate, oxalate, chloride, hydrochloride, bromide, hydrobromide, iodide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate. In certain aspects, the pharmaceutically acceptable salt is citrate.

In a further aspect, the present technology relates to a method of treating a neurodegenerative or metabolic disease or disorder, wherein the method comprises administering to the patient in need thereof the composition comprising the compound of Formula I to Formula VII, wherein the neurodegenerative disease, or metabolic disorder is a lysosomal storage disorder. In certain aspects the lysosomal storage disorder is selected from sphingolipidoses, mucopolysaccharidoses (MPS), lysosomal glycogen storage disorders, oligosaccharidoses, mucolipidoses, lipid storage disorders, glycoproteinoses, neuronal ceroid lipofuscinoses (NCL), peroxisomal biogenesis disorders (Zellweger spectrum disorders), and other lysosomal storage disorders.

In a further aspect, the present technology relates to a method of treating a neurodegenerative or metabolic disease or disorder, wherein the method comprises administering to the patient in need thereof the composition comprising the compound of Formula I to Formula VII, wherein the sphingolipidosis is selected from Gaucher disease type 1, Gaucher disease type 2, Gaucher disease type 3, Fabry disease, Niemann-pick disease type A, Niemann-pick disease type B, Niemann-pick disease type C, Tay-Sachs disease, Sandhoff disease, GM1 gangliosidosis, metachromatic leukodystrophy (MLD), Krabbe disease, and Farber lipogranulomatosis.

In a further aspect, the present technology relates to a method of treating a neurodegenerative or metabolic disease or disorder, wherein the method comprises administering to the patient in need thereof the composition comprising the compound of Formula I to Formula VII, wherein the mucopolysaccharidosis is selected from MPS I, Hurler syndrome, Hurler-Scheie syndrome, Scheie syndrome, MPS II, Hunter syndrome), MPS III, Sanfilippo syndrome type A, Sanfilippo syndrome type B, Sanfilippo syndrome type C, Sanfilippo syndrome type D, MPS IV, Morquio syndrome type A, Morquio syndrome type B), MPS VI, Maroteaux-Lamy syndrome, MPS VII, Sly syndrome, MPS IX, and Natowicz syndrome.

In a further aspect, the present technology relates to a method of treating a neurodegenerative or metabolic disease or disorder, wherein the method comprises administering to the patient in need thereof the composition comprising the compound of Formula I to Formula VII, wherein the lysosomal glycogen storage disorder is selected from Pompe disease and glycogen storage disease type II.

In a further aspect, the present technology relates to a method of treating a neurodegenerative or metabolic disease or disorder, wherein the method comprises administering to the patient in need thereof the composition comprising the compound of Formula I to Formula VII, wherein the oligosaccharidosis is selected from Schindler disease, Sialidosis Type I, Sialidosis type II, aspartylglucosaminuria, alpha-mannosidosis, beta-mannosidosis, and fucosidosis.

In a further aspect, the present technology relates to a method of treating a neurodegenerative or metabolic disease or disorder, wherein the method comprises administering to the patient in need thereof the composition comprising the compound of Formula I to Formula VII, wherein the mucolipidosis is selected from mucolipidosis II, I-cell disease, mucolipidosis III, pseudo-Hurler polydystrophy, and mucolipidosis IV.

In a further aspect, the present technology relates to a method of treating a neurodegenerative or metabolic disease or disorder, wherein the method comprises administering to the patient in need thereof the composition comprising the compound of Formula I to Formula VII, wherein the lipid storage disorder is selected from Wolman disease, cholesteryl ester storage disease, and Farber disease.

In a further aspect, the present technology relates to a method of treating a neurodegenerative or metabolic disease or disorder, wherein the method comprises administering to the patient in need thereof the composition comprising the compound of Formula I to Formula VII, wherein the glycoproteinosis is selected from alpha-mannosidosis, beta-mannosidosis, fucosidosis, aspartylglucosaminuria.

In a further aspect, the present technology relates to a method of treating a neurodegenerative or metabolic disease or disorder, wherein the method comprises administering to the patient in need thereof the composition comprising the compound of Formula I to Formula VII, wherein the neuronal ceroid lipofuscinosis is selected from infantile NCL, CLN1, CLN5, late infantile NCL, CLN2, juvenile NCL, CLN3, adult NCL, Kufs disease, CLN4, congenital NCL, and CLN10.

In a further aspect, the present technology relates to a method of treating a neurodegenerative or metabolic disease or disorder, wherein the method comprises administering to the patient in need thereof the composition comprising the compound of Formula I to Formula VII, wherein the peroxisomal biogenesis disorder is selected from Zellweger syndrome, neonatal adrenoleukodystrophy, and infantile Refsum disease.

In a further aspect, the present technology relates to a method of treating a neurodegenerative or metabolic disease or disorder, wherein the method comprises administering to the patient in need thereof the composition comprising the compound of Formula I to Formula VII, wherein the other lysosomal storage disorder is selected from Danon disease, cystinosis, sialic acid storage disease, Salla disease, lysosomal acid lipase deficiency (LAL-D), multiple sulfatase deficiency, pycnodysostosis, galactosialidosis, hyaluronidase deficiency (MPS IX), Hermansky-Pudlak syndrome, and pseudoxanthoma elasticum (PXE).

In a further aspect, the present technology relates to a method of treating a neurodegenerative or metabolic disease or disorder, wherein the method comprises administering to the patient in need thereof the composition comprising the compound of Formula I to Formula VII, wherein the neurodegenerative or metabolic disease or disorder is Niemann Pick Disease Type C, and wherein further the Niemann Pick disease Type C is Niemann Pick disease Type C 1 or Niemann Pick disease Type C 2.

In a further aspect, the present technology relates to a method of treating a neurodegenerative or metabolic disease or disorder, wherein the method comprises administering to the patient in need thereof the composition comprising the compound of Formula I to Formula VII in a therapeutically effective amount, wherein the therapeutically effective amount is between 1 mg and 5000 mg per day. In certain aspects, the therapeutically effective amount is between 500 mg and 2000 mg.

In a further aspect, the present technology relates to a method of treating a neurodegenerative or metabolic disease or disorder, wherein the method comprises administering to the patient in need thereof the composition comprising the compound of Formula I to Formula VII, wherein the therapeutically effective amount is administered in 1 to 2 unit doses. In certain aspects, the therapeutically effective amount is administered in 1 unit dose.

In a further aspect, the present technology relates to a method of treating a neurodegenerative or metabolic disease or disorder, wherein the method comprises administering to the patient in need thereof the composition comprising the compound of Formula I to Formula VII, wherein the method further comprises administering a therapeutically effective amount of at least one additional active pharmaceutical ingredient selected from bimoclomol, miglustat, eligustat, N-acetyl-L-leucine, arimoclomol, and 2-hydroxypropyl-β-cyclodextrin, and combinations thereof. In a still further aspect, the at least one additional active pharmaceutical ingredient is selected from N-acetyl-L-leucine, 2-hydroxypropyl-β-cyclodextrin, and combinations thereof.

In certain aspects, the present disclosure provides a method of treating Niemann-Pick Disease, Type C, in a patient in need thereof less than 2 years of age, the method comprising the steps of: administering to the patient in need thereof a composition comprising a therapeutically effective amount of a compound having a structure of Formula I to Formula VII, or a pharmaceutically acceptable salt thereof, wherein the therapeutically effective amount ranges from about 0.1 mg/kg/day to about 10 mg/kg/day of arimoclomol freebase.

In a further aspect, the present disclosure provides a method of treating Niemann-Pick Disease, Type C, in a patient in need thereof less than 2 years of age, the method comprising the steps of: administering to the patient in need thereof a composition comprising a therapeutically effective amount of a compound having a structure of Formula I to Formula VII, or a pharmaceutically acceptable salt thereof, wherein the therapeutically effective amount ranges from about 1 mg/kg/day to about 8 mg/kg/day. In an embodiment, the therapeutically effective amount is 6 mg/kg/day of arimoclomol freebase.

In a still further aspect, the present disclosure provides a method of treating Niemann-Pick Disease, Type C, in a patient in need thereof less than 2 years of age, the method comprising the steps of: administering to the patient in need thereof a composition comprising a therapeutically effective amount of a compound having a structure of Formula I to Formula VII, or a pharmaceutically acceptable salt thereof, wherein the therapeutically effective amount is administered in 1 to 3 unit doses. In an embodiment, the therapeutically effective amount is administered in 3 unit doses. In an alternative embodiment, the unit dosage is 2 mg/kg/day of arimoclomol freebase.

In yet another aspect, the present disclosure provides a method of treating Niemann-Pick Disease, Type C, in a patient in need thereof less than 2 years of age, the method comprising the steps of: administering to the patient in need thereof a composition comprising a therapeutically effective amount of a compound having a structure of Formula I to Formula VII, or a pharmaceutically acceptable salt thereof, wherein the composition is liquid oral dosage formulation. In an alternative embodiment, the liquid oral dosage formulation comprises about 0.1 mg/mL to about 5 mg/mL of the compound of Formula I-VII, or pharmaceutically acceptable salt thereof. In an embodiment, the liquid oral dosage formulation comprises about 3.1 mg/mL of the compound of Formula I-VII, or pharmaceutically acceptable salt thereof. In a further aspect, the present disclosure provides a method of treating Niemann-Pick Disease, Type C, in a patient in need thereof less than 2 years of age, the method comprising the steps of: administering to the patient in need thereof a composition comprising a therapeutically effective amount of a compound having a structure of Formula I to Formula VII, or a pharmaceutically acceptable salt thereof, wherein the composition is liquid oral dosage formulation wherein the liquid oral dosage formulation made by emptying the contents of a 47 mg of arimoclomol citrate capsule into 15 mL of water. The liquid oral formulation may be administered orally or through a feeding tube. In this embodiment, the amount of the liquid oral formulation administered to the patient in need thereof is measured in 0.2 mL increments and remaining solution is discarded and not saved for later use. Alternatively the contents of a 31 mg arimoclomol citrate capsule may be emptied into 10 mL of water; alternatively, the contents of a 62 mg of arimoclomol citrate capsule may be emptied into 20 mL of water.

### BRIEF SUMMARY OF THE DRAWINGS

FIG. 1 shows the plasma concentration of arimoclomol following administration of ethyl-O—CO-ARIM compared to administration of arimoclomol for 6 hours.

FIG. 2 shows the plasma concentration of arimoclomol following administration of hexyl-O—CO-ARIM compared to administration of arimoclomol for 6 hours.

FIG. 3 shows the plasma concentration of arimoclomol following administration of benzoyl-ARIM and cinnamoyl-ARIM compared to administration of arimoclomol for 6 hours.

FIG. 4 shows the plasma concentration of arimoclomol following administration of ethyl-O—CO-ARIM compared to administration of arimoclomol for 6 hours.

FIG. 5 shows the plasma concentration of arimoclomol following administration of butyryl-ARIM and Val-ARIM compared to administration of arimoclomol for 6 hours.

FIG. 6 shows the plasma concentration of arimoclomol following administration of ethylsuccinyl-ARIM compared to administration of arimoclomol for 6 hours.

FIG. 7 shows the plasma concentration of arimoclomol following administration of glutaryl-ARIM and ethyloxyglutaryl-ARIM compared to administration of arimoclomol for 6 hours.

FIG. 8 shows the plasma concentration of arimoclomol following administration of octanoyl-ARIM and hexanoyl-ARIM compared to administration of arimoclomol for 6 hours.

FIG. 9 shows the plasma concentration of arimoclomol following administration of decanoyl-ARIM and dodecanoyl-ARIM compared to administration of arimoclomol for 6 hours.

FIG. 10 shows the plasma concentration of arimoclomol following administration of amino-PEG2-acetyl-ARIM and sorboyl-ARIM compared to administration of arimoclomol for 6 hours.

FIG. 11 shows the plasma concentration of arimoclomol following administration of anisoyl-ARIM compared to administration of arimoclomol for 6 hours.

FIG. 12 shows the plasma concentration of arimoclomol following administration of amino-PEG4-propanoyl-ARIM compared to administration of arimoclomol for 6 hours.

FIG. 13 shows the plasma concentration of arimoclomol following administration of Phe-ARIM and Tyr-ARIM compared to administration of arimoclomol for 6 hours.

FIG. 14 shows the plasma concentration of arimoclomol following administration of succinyl-ARIM compared to administration of arimoclomol for 6 hours.

FIG. 15 shows the plasma concentration of arimoclomol following administration of adipoyl-ARIM compared to administration of arimoclomol for 6 hours.

FIG. 16 shows the plasma concentration of arimoclomol following administration of maleoyl-ARIM compared to administration of arimoclomol for 6 hours.

FIG. 17 shows the plasma concentration of arimoclomol following administration of benzoyl-NH—CO-ARIM compared to administration of arimoclomol for 6 hours.

FIG. 18 shows the plasma concentration of arimoclomol following administration of N-methoxy-N(Me)CO—OCH2-ARIM compared to administration of arimoclomol for 6 hours.

FIG. 19 shows the plasma concentration of arimoclomol following administration of ARIM-succinyl-ARIM compared to administration of arimoclomol for 6 hours.

FIG. 20 shows the plasma concentration of arimoclomol following administration of ARIM-glutaryl-ARIM compared to administration of arimoclomol for 6 hours.

FIG. 21 shows the plasma concentration of arimoclomol following administration ARIM-adipoyl-ARIM compared to administration of arimoclomol for 6 hours.

FIG. 22 shows the plasma concentration of arimoclomol following administration of ARIM-terephthaloyl-ARIM compared to administration of arimoclomol for 6 hours.

FIG. 23 shows the plasma concentration of arimoclomol following administration of ARIM-(cyclohexane-1,3-dicarbonyl)-ARIM compared to administration of arimoclomol for 6 hours.

### DETAILED DESCRIPTION

The present disclosure provides prodrug compounds of arimoclomol having a structure of Formula I, their pharmaceutically acceptable salts thereof, compositions comprising the same, and methods of treating neurodegenerative and/or metabolic disorders by administering therapeutically effective amounts of the same.

Currently, arimoclomol is administered either in the free base form or as a pharmaceutical salt. These compounds are immediately released from the pharmaceutical composition and reach peak plasma levels in about 0.5-4 hours depending on whether the patient is fasting or has recently eaten. Effectively treating diseases such as Niemann-Pick disease Type C and Gaucher Disease require administering 2-3 unit doses of arimoclomol per day in order to maintain effective plasma levels in the patient. This leads to inconsistent plasma levels that reach a maximum and then decrease until the next dose is administered. This results in less effective treatment and progression of the disease

The inventors discovered that arimoclomol can be modified at either the 2-hydroxy or N-piperidine position with substituent groups that are cleaved by enzymes which release unconjugated arimoclomol . . . .

The presently described compounds unexpectedly display much better pharmacokinetics characterized by either lower maximum plasma concentrations (Cₘₐₓ) or Cₘₐₓ values comparable to unmodified arimoclomol, longer times to maximum plasma concentration (Tₘₐₓ), and longer half-life (t½). This results in a longer elimination phase with higher plasma concentrations over a longer period of time as measured by Area Under the Curve (AUC). These improved pharmacokinetics allow for dosages that result in therapeutically effective plasma levels for longer periods of time per dose, meaning patients need fewer doses per day, even only a single dose per day.

Reference will now be made in detail to exemplary embodiments of the claimed invention. While the claimed invention will be described in conjunction with the exemplary embodiments, it will be understood that it is not intended to limit the claimed invention to those embodiments. To the contrary, it is intended to cover alternatives, modifications, and equivalents, as may be included within the spirit and scope of the claimed invention, as defined by the appended claims.

Those of ordinary skill in the art may make modifications and variations to the embodiments described herein without departing from the spirit or scope of the claimed invention. In addition, although certain methods and materials are described herein, other methods and materials that are similar or equivalent to those described herein can also be used to practice the claimed invention.

In addition, any of the compositions or methods provided, disclosed, or described herein can be combined with one or more of any of the other compositions and methods provided, disclosed, or described herein.

### Definitions

“A” and “an” as it relates to the present technology, means the singular form, but includes the plural form unless clear from the context.

“About” as it related to the present technology means, as it applied to measured quantities, +/−10% of the stated measured value; for example “about 100 mg” means 100 mg+/−10%, i.e. 90-110 mg. Unless specifically stated or obvious from context, as used herein, the term “about” is understood as within a range of normal tolerance in the art, for example, within two standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

As used herein, the term “or” means, and is used interchangeably with, the term “and/or,” unless context clearly indicates otherwise.

As used herein, the term “such as” means, and is used interchangeably with, the phrase “such as, for example” or “such as but not limited.”

As used herein, the term “subject” means a human or animal, including but not limited to a human or animal patient.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless otherwise defined, all technical and scientific terms used herein have the same meaning as commonly understood by one of ordinary skill in the art to which the methods described herein belong. Any reference to standard methods (e.g., ASTM, TAPPI, AATCC, etc.) refers to the most recent available version of the method at the time of filing of this disclosure unless otherwise indicated.

“Arimoclomol” as it related to the present technology means N-[2-hydroxy-3-(1-piperidinyl)-propoxy]-pyridine-1-oxide-3-carboximidoyl chloride, its stereoisomers and the acid addition salts thereof. Stereoisomers of arimoclomol include, but are not limited to (+)-(R)—N-[2-hydroxy-3-(1-piperidinyl)-propoxy]-pyridine-1-oxide-3-carboximidoyl chloride, (−)-(S)—N-[2-hydroxy-3-(1-piperidinyl)-propoxy]-pyridine-1-oxide-3-carboximidoyl chloride,(Z)—(R)—N-[2-hydroxy-3-(1-piperidinyl)-propoxy]-pyridine-1-oxide-3-carboximidoyl chloride,(E)-(R)—N-[2-hydroxy-3-(1-piperidinyl)-propoxy]-pyridine-1-oxide-3 carboximidoyl chloride,(Z)—(S)—N-[2-hydroxy-3-(1-piperidinyl)-propoxy]-pyridine-1-oxide-3-carboximidoyl chloride; and (E)-(S)—N-[2-hydroxy-3-(1-piperidinyl)-propoxy]-pyridine-1-oxide-3-carboximidoyl chloride.

“ARIM” as it relates to the present technology refers to an arimoclomol moiety that is part of a larger molecular structure.

“ELIG” as it relates to the present technology refers to an eliglustat moiety that is part of a larger molecular structure.

“MIG” as it relates to the present technology refers to an miglustat moiety that is part of a larger molecular structure.

“Cholesterol,” as it relates to the present technology means free cholesterol or cholest-5-en-3β-ol. “Cholesterol” as it relates to the present technology includes, and may be used interchangeably with “unesterified cholesterol” as defined below.

“Coordinated Lysosomal Expression and Regulation” and/or “CLEAR” as it relates to the present technology means a transcriptional network directed primarily by certain transcription factors including, for example, TFEB and MiT/TFE factors (TFE3, MITF, TFEC) that bind to promoter sequences enriched in genes related to lysosomal and autophagic functions to coordinate processes that include but are not limited to lysosomal biogenesis, autophagy, autophagosome-lysosome fusion, lipid catabolism, and cellular stress-adaptation pathways.

“Drug” or “medicament” as it related to the present technology includes biologically, physiologically, or pharmacologically active substances that act locally or systemically in the human or animal body.

“Esterified cholesterol” as it relates to the present technology means a form of cholesterol where the hydroxyl group of the cholesterol molecule has been conjugated with a fatty acid to form an ester-bond that links the cholesterol molecule and the fatty acid. In most animal tissues, an enzyme acyl-CoA:cholesterol acyltransferase (ACAT) or sterol O-acyl-transferase (SOAT) synthesizes cholesterol esters from CoA esters of fatty acids and cholesterol. ACAT exists in two forms, both of which are intracellular enzymes found in the endoplasmic reticulum and are characterized by multiple transmembrane domains and a catalytic histidine residue in a hydrophobic domain.

“Niemann-Pick disease type C Clinical Severity Scale” and/or “NPCCSS” as it relates to the present technology means a composite clinical severity scale (hereafter “NPCCSS”; see Yanjanin et al.). A full “17-domain NPCCSS score” incorporates clinical signs and symptoms in nine major (ambulation, cognition, eye movement, fine motor, hearing, memory, seizures, speech, swallowing) and eight minor (auditory brainstem response, behavior, gelastic cataplexy, hyperreflexia, incontinence, narcolepsy, psychiatric, respiratory problems) domains to determine a score which describes the severity of the subject's NPC progression (a higher score, the more progressed/severe the disease is). An abridged “5-domain NPCCSS score” is successfully used by clinicians and incorporates clinical signs and symptoms from the major domains of ambulation, cognition, fine motor, speech and swallowing (see Cortina-Borja). A “4-domain NPCCSS score” is used based on Ambulation, Fine Motor Skills, Speech, and Swallow domains.

“Impaired cholesterol trafficking” as it relates to the present technology means a reduced ability to remove cholesterol from the cell through the normal lysosomal pathway.

“Including,” as it relates to the present technology means, and is used interchangeably with, the phrase “including but not limited to.”

“Isoform 1” as it relates to the present technology means a mature form of NPC1 protein

“Lipid storage disorders” or “lipidoses” are a subgroup of the lysosomal storage disorders in which harmful amounts of lipids accumulate in the intracellular space due to reduced expression or function of the enzymes needed to metabolize lipids. Over time, this excessive storage of lipids can cause permanent cellular and tissue damage, particularly in the brain, peripheral nervous system, liver, spleen and bone marrow.

Lipids are a broad group of naturally-occurring molecules which include fats, waxes, sterols, fat-soluble vitamins (such as vitamins A, D, E and K), monoglycerides, diglycerides, phospholipids, and others. The main biological functions of lipids include energy storage, as structural components of cell membranes, and as important signaling molecules.

Lipids may be broadly defined as hydrophobic or amphiphilic small molecules; the amphiphilic nature of some lipids allows them to form structures such as vesicles, liposomes, or membranes in an aqueous environment. Biological lipids originate entirely or in part from two distinct types of biochemical subunits: ketoacyl and isoprene groups. Using this approach, lipids may be divided into eight categories: fatty acyls, glycerolipids, glycerophospholipids, sphingolipids, saccharolipids and polyketides (derived from condensation of ketoacyl subunits); and sterol lipids and prenol lipids (derived from condensation of isoprene subunits).

Although the term lipid is sometimes used as a synonym for fats, fats are a subgroup of lipids called triglycerides. Lipids also encompass molecules such as fatty acids and their derivatives (including tri-, di-, and monoglycerides and phospholipids), as well as other sterol-containing metabolites such as cholesterol.

Several lysosomal storage disorders characterized by the accumulation of lipids (i.e., lipid storage disorders) have been characterized; these are outlined herein below.

### Pathophysiology of Niemann-Pick Disease

“Lysosomal Sphingolipid Hydrolysis” as it relates to the present technology means a multitude of enzymes are involved in the lysosomal catabolism of sphingolipids (or glycophingolipids). These enzymes, or more specifically hydrolases, are each responsible for the degradation of a specific sphingolipid.

The lysosomal sphingolipid hydrolases interact with sphingolipid activator proteins (SAP or saposins) to stimulate the activity of said hydrolases. SAPs are considered to facilitate the enzyme/substrate interaction between water-soluble enzymes and membrane-bound substrates.

Further, the lipid composition of late endosomal and lysosomal compartments is characterized by the presence of negatively charged phospholipids such as BMP and PI (phosphatidylinositol), which also stimulates the activity of some hydrolases. The BMP-dependent lysosomal hydrolases include sialidase, α-galactosidase A, glucosylceramidase, 3-galactosylceramidase, arylsulfatase A, acid ceramidase and Sphingomyelinase.

“Maturation,” “maturing,” or “mature” as it relates to the present technology means the process and/or characteristic of a protein reaching its folded state and optionally being subjected to post-translational modifications including, for example, glycosylation.

“Mechanism of Action of Arimoclomol” or “Arimoclomol MOA” as it related to the present technology means the biological pathway by which arimoclomol treats the NPC Disease State. Arimoclomol targets NPC etiology by both NPC1-independent and NPC1-dependent pathways: (1) via the NPC1-independent pathway, arimoclomol upregulates expression of certain CLEAR genes other than NPC1, thereby improving overall cell health by, for example, increasing lysosomal biogenesis and/or autophagy flux; (2) via the NPC1-dependent pathway, CLEAR gene upregulation increases production of the NPC1 protein. Though still mutated, overproducing the reduced function protein improves lysosomal function and cholesterol elimination.

“Myelin Basic Protein” and/or “MBP” as it relates to the present technology means a protein believed to be important in the process of myelination of nerves in the nervous system. The myelin sheath is a multi-layered membrane, unique to the nervous system, that functions as an insulator to greatly increase the velocity of axonal impulse conduction. MBP maintains the correct structure of myelin, interacting with the lipids in the myelin membrane.

“Patient” as it relates to the present technology means a human or animal subject in need of treatment.

“Pharmaceutically acceptable salt” as it relates to the present technology means a salt which is not harmful to the subjects. Such salts include pharmaceutically acceptable basic or acid addition salts as well as pharmaceutically acceptable metal salts, ammonium salts and alkylated ammonium salts.

Acid addition salts include, but are not limited to, salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 66, 2, (1977) which is incorporated herein by reference.

Other pharmaceutically acceptable salts include, but are not limited to, acetate, l-aspartate, besylate, bicarbonate, carbonate, d-camsylate, l-camsylate, citrate, edisylate, formate, fumarate, gluconate, hydrobromide/bromide, hydrochloride/chloride, d-lactate, l-lactate, d,l-lactate, d,l-malate, l-malate, mesylate, pamoate, phosphate, succinate, sulfate, bisulfate, d-tartrate, l-tartrate, d,l-tartrate, meso-tartrate, benzoate, gluceptate, d-glucuronate, hibenzate, isethionate, malonate, methylsulfate, 2-napsylate, nicotinate, nitrate, orotate, stearate, tosylate, thiocyanate, acefyllinate, aceturate, aminosalicylate, ascorbate, borate, butyrate, camphorate, camphocarbonate, decanoate, hexanoate, cholate, cypionate, dichloroacetate, edentate, ethyl sulfate, furate, fusidate, galactarate, galacturonate, gallate, gentisate, glutamate, glutarate, glycerophosphate, heptanoate, hydroxybenzoate, hippurate, phenylpropionate, iodide, xinafoate, lactobionate, laurate, maleate, mandelate, methanesulfonate, myristate, napadisilate, oleate, oxalate, palmitate, picrate, pivalate, propionate, pyrophosphate, salicylate, salicylsulfate, sulfosalicylate, tannate, terephthalate, thiosalicylate, tribrophenate, valerate, valproate, adipate, 4-acetamidobenzoate, camsylate, octanoate, estolate, esylate, glycolate, thiocyanate, or undecylenate. In the preferred embodiments, the anionic salt form is selected from chloride, hydrogen carbonate (bicarbonate), iodide, bromide, citrate, acetate, formate, salicylate, hydrogen sulfate (bisulfate), hydroxide, nitrate, hydrogen sulfite (bisulfite), propionate, benzene sulfonate, hypophosphite, phosphate, bromate, iodate, chlorate, fluoride, nitrite.

“Rearing” as it relates to the present technology means a functional behavior in rodents that plays an important role in exploring and interacting with the environment. As such, rearing is a complex behavior that involves aspects such as locomotion, balance, exploratory drive, spatial awareness, cognitive mapping, sequence learning, and decision making. Therefore, the ability to rear is not simply an indicator of muscle function but a broader marker of brain health in rodents. The same brain regions (particularly the cerebellum, hippocampus, and midbrain) that are responsible for controlling rearing behavior in rodents are involved in recruiting muscles during movements related to fine motor function and swallow in humans. Rearing activity in NPC mice is therefore an appropriate indicator of neuronal health in brain regions relevant to functional endpoints like the Fine Motor Skills and Swallow domains of the NPCCSS in human NPC patients

“Saposin Deficiency” as it relates to the present technology means a disease (in both humans and mice) caused by prosaposin/saposin deficiencies which leads to severe neurological deficits.

Human patients with point mutations in the saposin A, B and C show phenotypes of Krabbe disease, metachromatic leukodystrophy and Gaucher disease, indicating that their primary in vivo substrates are galactosylceramide, sulfatide and glucosylceramide, respectively.

Krabbe disease, atypical, due to saposin A deficiency: An inherited biochemical disorder which results in neurological regression within a few months of birth. Death usually occurs during the first few years of life. The disorder is similar to Krabbe disease but is differentiated by the genetic origin of the biochemical defect. Krabbe disease involves a defect in the galactocerebrosidase gene whereas atypical Krabbe disease involves a defect in the prosaposin gene which causes a deficiency of saposin A.

Saposin B, previously known as SAP-1 and sulfatide activator, stimulates the hydrolysis of a wide variety of substrates including cerebroside sulfate, GM1 ganglioside, and globotriaosylceramide by arylsulfatase A, acid beta-galactosidase, and alpha-galactosidase, respectively. Human saposin B deficiency, transmitted as an autosomal recessive trait, results in tissue accumulation of cerebroside sulfate and a clinical picture resembling metachromatic leukodystrophy (activator-deficient metachromatic leukodystrophy) although with normal arylsulfatase activity. Saposin B deficiency is a heterogeneous disease with a spectrum similar to that in metachromatic leukodystrophy.

Saposin (SAP-) C is required for glucosylceramide degradation, and its deficiency results in a variant form of Gaucher disease; non-neuronopathic Gaucher disease due to SAP-C deficiency. Very high levels of chitotriosidase activity, chemokine CCL18, and increased concentration of glucosylceramide in plasma and normal β-glucosidase activity in skin fibroblasts are observed in the patients. A missense mutation, p.L349P, located in the SAP-C domain and another mutation, p.M1L, located in the initiation codon of the prosaposin precursor protein has been identified.

In a few non-neuronopathic Gaucher patients, a mutation in both Saposin C and saposin D has been identified.

Combined saposin C and D deficiencies in mice lead to a neuronopathic phenotype with glucosylceramide and alpha-hydroxy ceramide accumulation.

In mice, saposin D deficiency is associated with ceramide accumulation, partial loss of Purkinje cells and impaired urinary system function. This phenotype does not mimic the embryonic lethality exhibited by mice with complete deficiency of acid ceramidase, saposin D's cognate enzyme

Two mutations are known in humans that result in complete inactivation of all four saposins and prosaposin. Total saposin deficiency is a devastating disease with involvement of multiple organs and multiple sphingolipids. Combined saposin deficiency (or prosaposin deficiency) has been reported in a case presenting with a severe neurovisceral dystrophy which caused death as a neonate. Multiple sphingolipids were elevated in the urine, with globotriaosylceramide showing the greatest increase. A novel mutation in the PSAP gene was identified, being homozygous for a splice-acceptor site mutation two bases upstream of exon 10. This mutation led to a premature stop codon and yielded low levels of transcript.

“Transcription Factor EB” as it relates to the present technology means protein that in humans is encoded by the TFEB gene. TFEB is a master gene for lysosomal biogenesis. It encodes a transcription factor that coordinates expression of lysosomal hydrolases, membrane proteins and genes involved in autophagy. Upon nutrient depletion and under aberrant lysosomal storage conditions such as in lysosomal storage diseases, TFEB translocate from the cytoplasm to the nucleus, resulting in the activation of its target genes.

“Treating” as it related to the present technology means any of the following: (1) delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

“Unesterified cholesterol” as it relates to the present technology means “free cholesterol,” or cholest-5-en-3β-ol.

“Upregulate,” “upregulation,” or “upregulates” as it relates to the present technology means a process by which gene expression increases. Upregulation can be determined by measuring an increase in the presence of nucleotide strands such as DNA and/or RNA or an increase of the quantity of protein encoded by the gene which is expressed by the cell.

### Hydroxylamine Compounds

In an embodiment, the present disclosure provides a genus of compounds having a structure of Formula I:

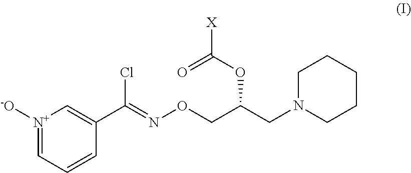

wherein X is selected from the group consisting of R, [O—R], [NR¹R²], and [NRH]
wherein O is oxygen, N is nitrogen, H is hydrogen, and
R, R¹ and R² are independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

“Pharmaceutically acceptable salt” as it relates to the present technology means a salt which is not harmful to the subjects. Such salts include pharmaceutically acceptable basic or acid addition salts as well as pharmaceutically acceptable metal salts, ammonium salts and alkylated ammonium salts.

Acid addition salts include, but are not limited to, salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 66, 2, (1977) which is incorporated herein by reference.

Other pharmaceutically acceptable salts include, but are not limited to, acetate, l-aspartate, besylate, bicarbonate, carbonate, d-camsylate, l-camsylate, citrate, edisylate, formate, fumarate, gluconate, hydrobromide/bromide, hydrochloride/chloride, d-lactate, l-lactate, d,l-lactate, d,l-malate, l-malate, mesylate, pamoate, phosphate, succinate, sulfate, bisulfate, d-tartrate, l-tartrate, d,l-tartrate, meso-tartrate, benzoate, gluceptate, d-glucuronate, hybenzate, isethionate, malonate, methylsulfate, 2-napsylate, nicotinate, nitrate, orotate, stearate, tosylate, thiocyanate, acefyllinate, aceturate, aminosalicylate, ascorbate, borate, butyrate, camphorate, camphocarbonate, decanoate, hexanoate, cholate, cypionate, dichloroacetate, edentate, ethyl sulfate, furate, fusidate, galactarate, galacturonate, gallate, gentisate, glutamate, glutarate, glycerophosphate, heptanoate, hydroxybenzoate, hippurate, phenylpropionate, iodide, xinafoate, lactobionate, laurate, maleate, mandelate, methanesulfonate, myristate, napadisilate, oleate, oxalate, palmitate, picrate, pivalate, propionate, pyrophosphate, salicylate, salicylsulfate, sulfosalicylate, tannate, terephthalate, thiosalicylate, tribrophenate, valerate, valproate, adipate, 4-acetamidobenzoate, camsylate, octanoate, estolate, esylate, glycolate, thiocyanate, or undecylenate. In the preferred embodiments, the anionic salt form is selected from chloride, hydrogen carbonate (bicarbonate), iodide, bromide, citrate, acetate, formate, salicylate, hydrogen sulfate (bisulfate), hydroxide, nitrate, hydrogen sulfite (bisulfite), propionate, benzene sulfonate, hypophosphite, phosphate, bromate, iodate, chlorate, fluoride, nitrite.

In a further embodiment, the present disclosure provides a compound of Formula I, wherein the compound has a structure of Formula IA:

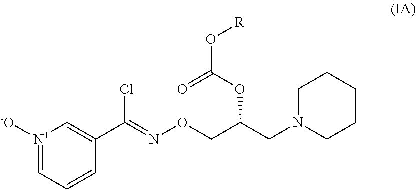

where R is selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a still further embodiment, the present disclosure provides a compound of Formula IA, wherein the compound of IA is a compound having a structure selected from:

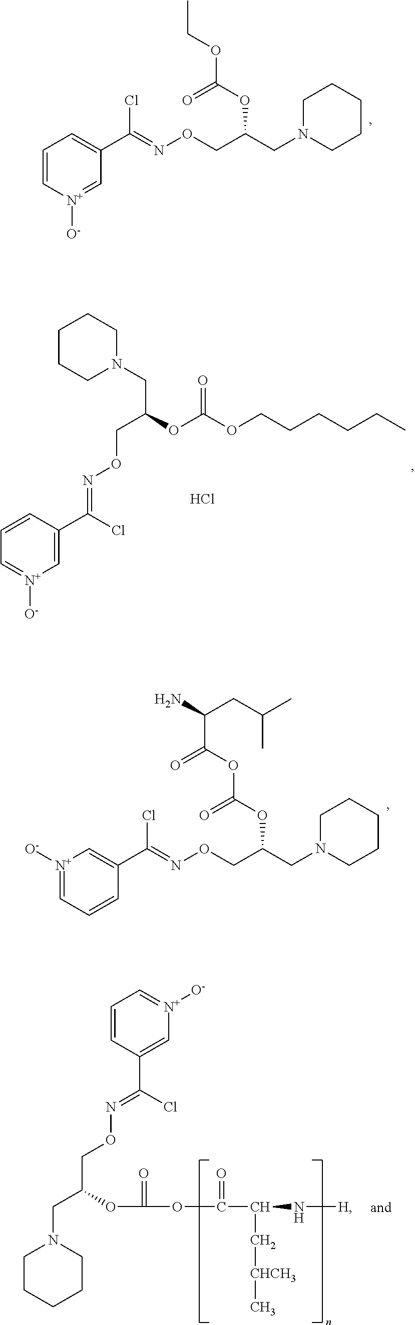

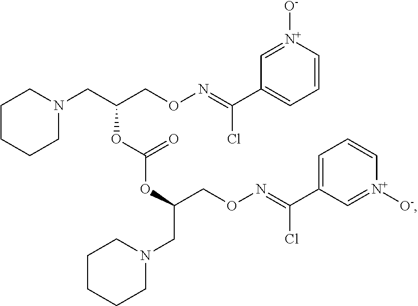

wherein n is 1-50;
or a pharmaceutically acceptable salt thereof.

In a still further embodiment, the present disclosure provides a compound of Formula IA, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In a further embodiment, the present disclosure provides a compound of Formula I, wherein the compound of IB is a compound having a structure of Formula IB:

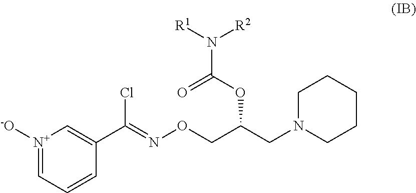

wherein R¹ and R² are independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a still further embodiment, the present disclosure provides a compound of Formula IB, wherein the compound of IB is a compound having a structure selected from:

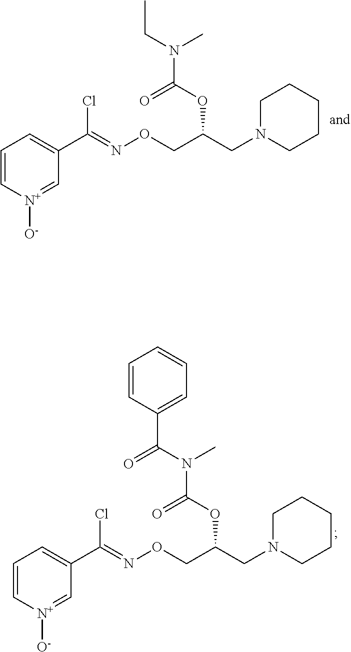 [SMILES: CCN(C)C(=O)O[C@@H](CO/N=C(\Cl)c1ccc[n+]([O-])c1)CN1CCCCC1 ; CN(C(=O)O[C@@H](CO/N=C(\Cl)c1ccc[n+]([O-])c1)CN1CCCCC1)C(=O)c1ccccc1]

or a pharmaceutically acceptable salt thereof.

In a further embodiment, the present disclosure provides a compound of Formula I, wherein the compound of Formula I is a compound of Formula IC:

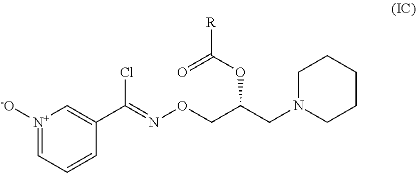

wherein R is selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a still further embodiment, the present disclosure provides a compound of Formula IC, wherein the compound of IC is a compound having a structure selected from:

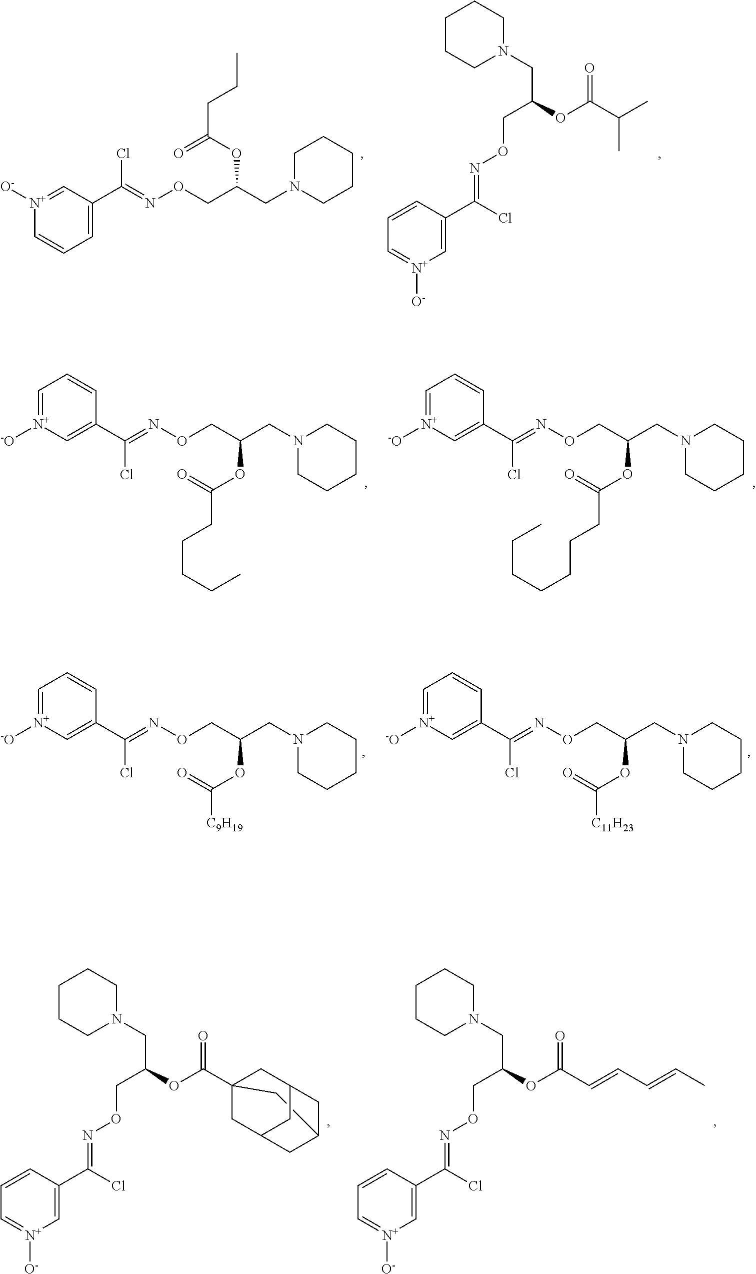

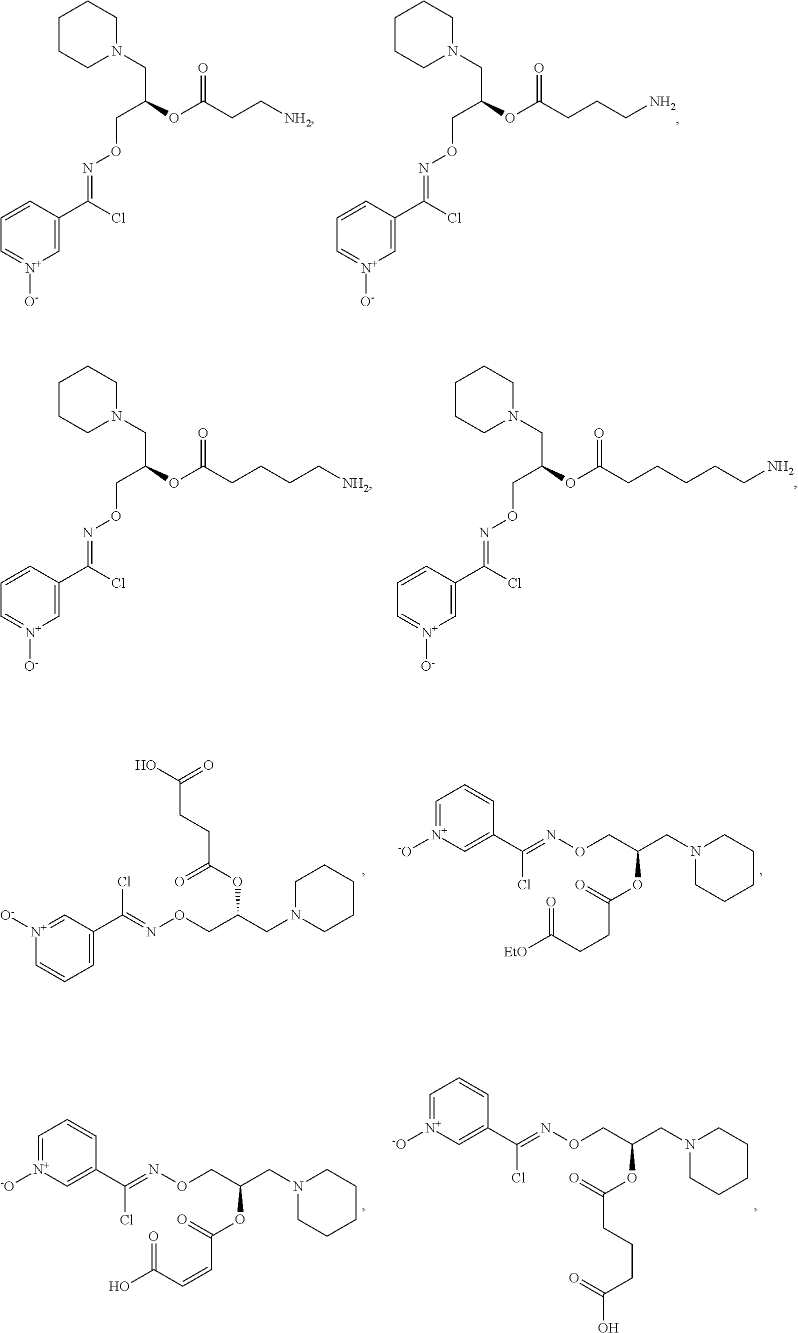

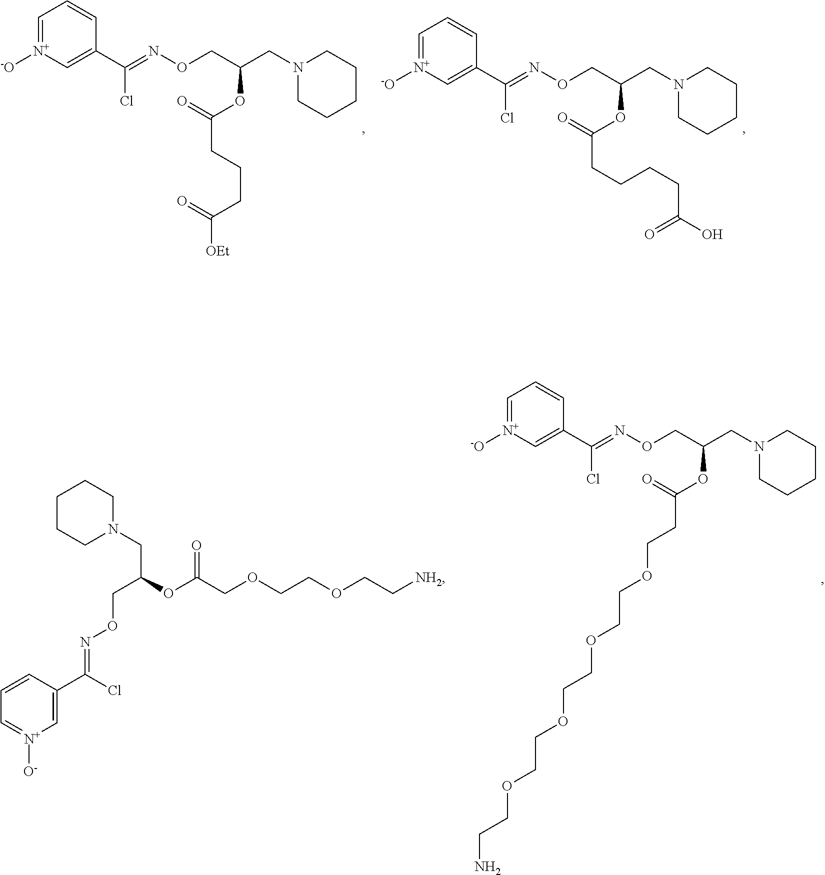

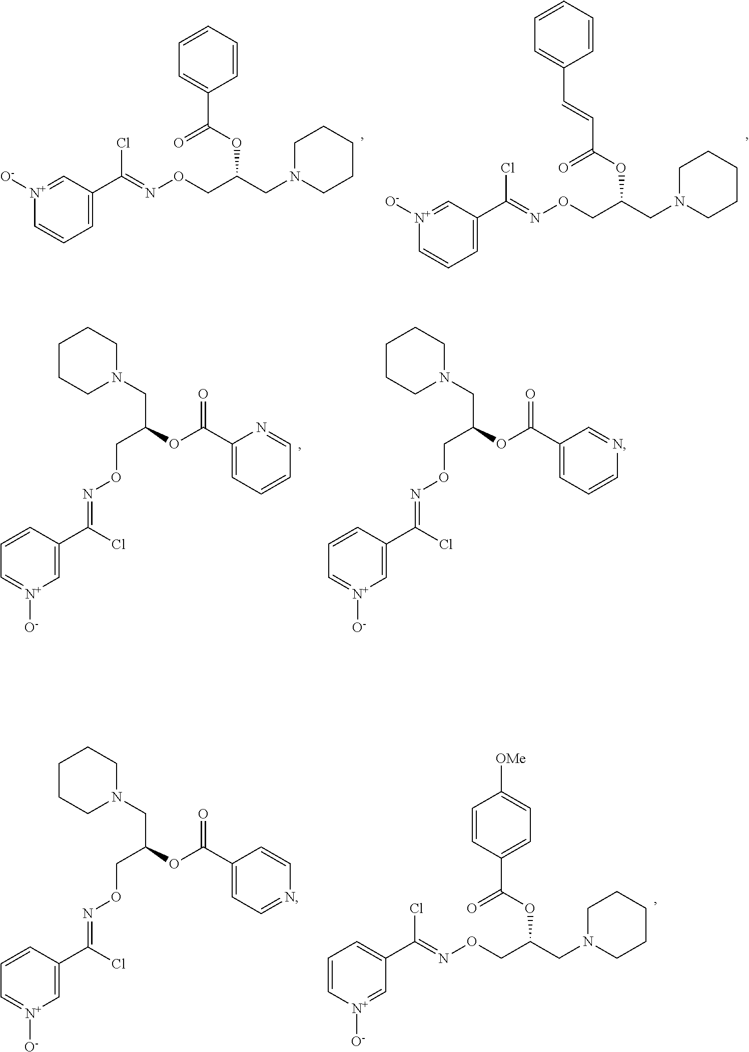

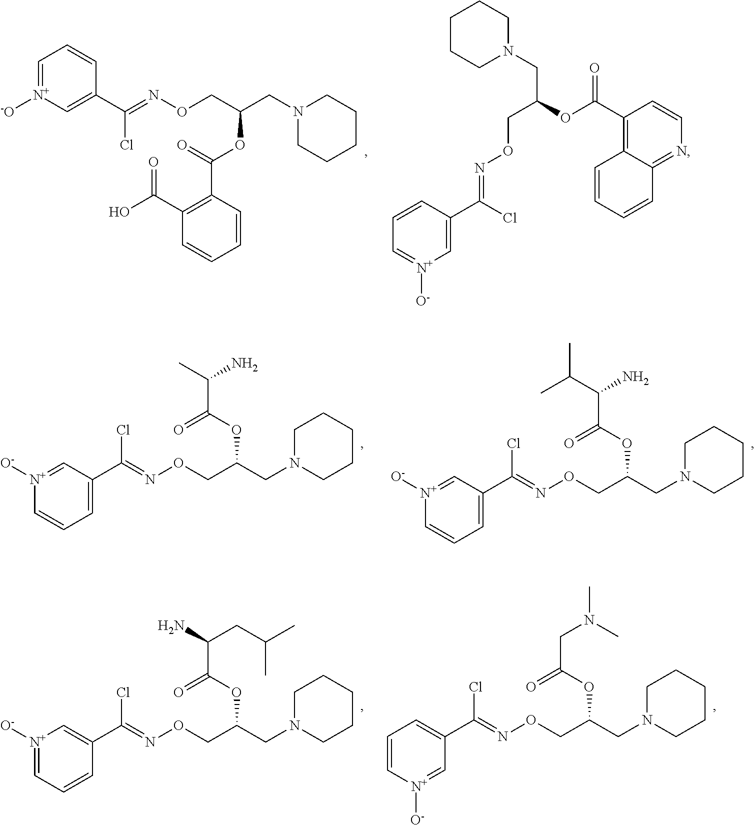

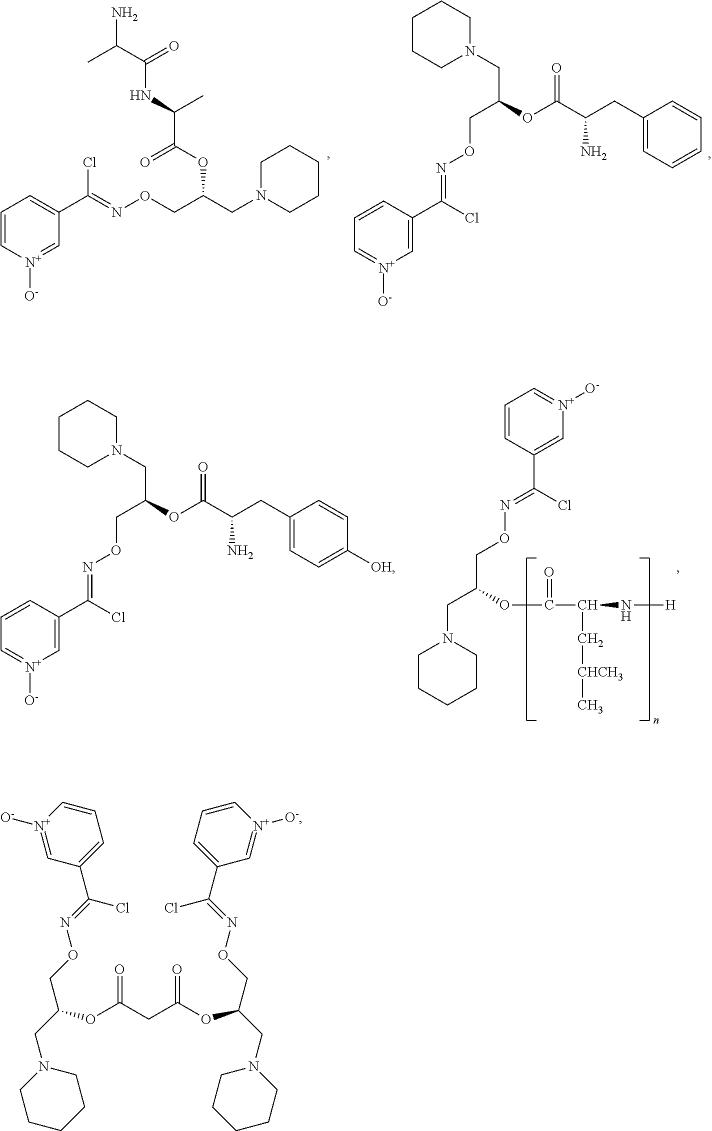

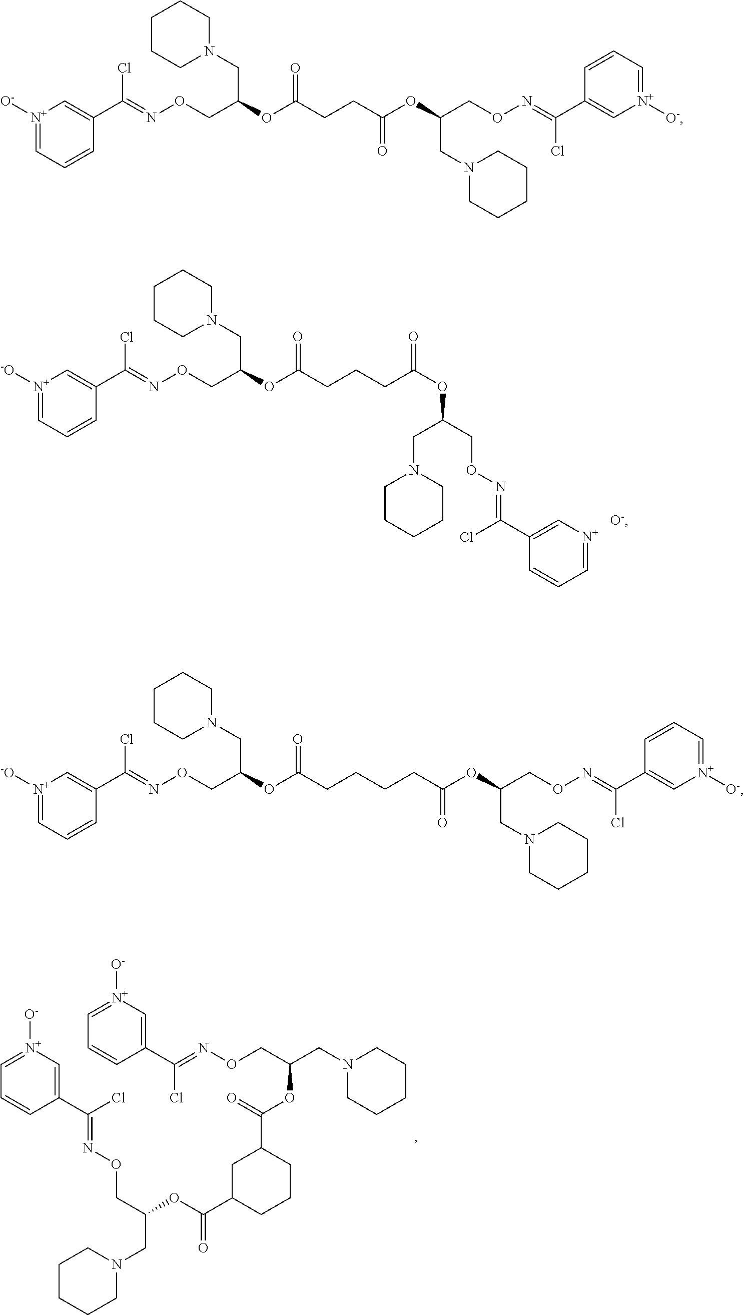

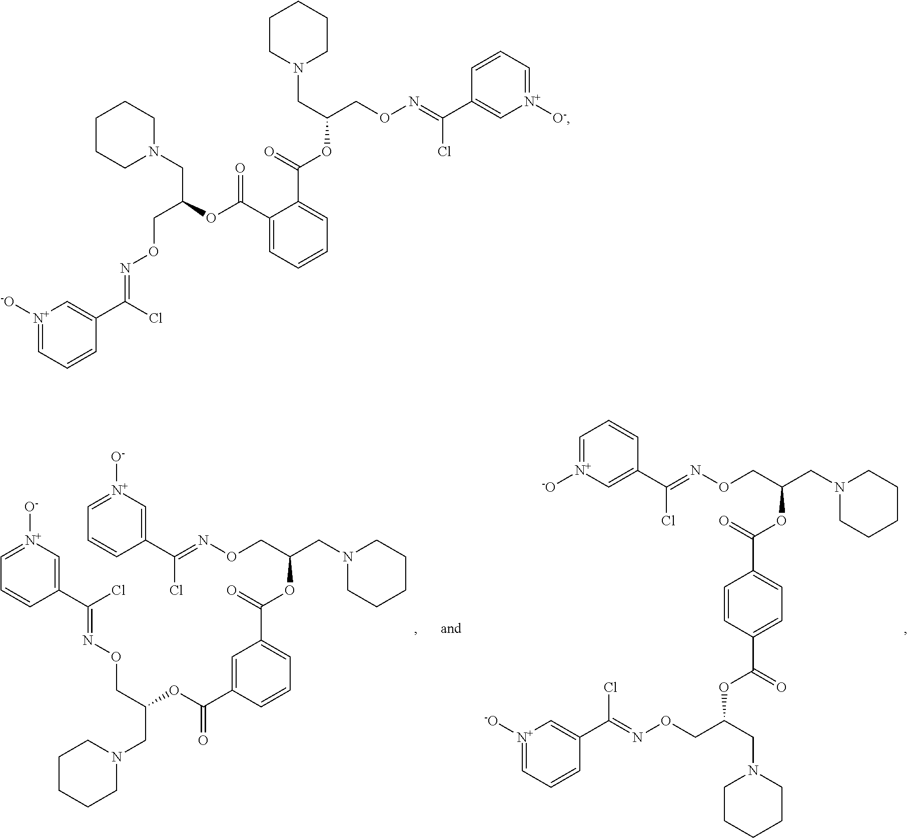

wherein n is 1-50;
or a pharmaceutically acceptable salt thereof.

In a still further embodiment, the present disclosure provides a compound of Formula IC, wherein the compound of IC is a compound having a structure selected from:

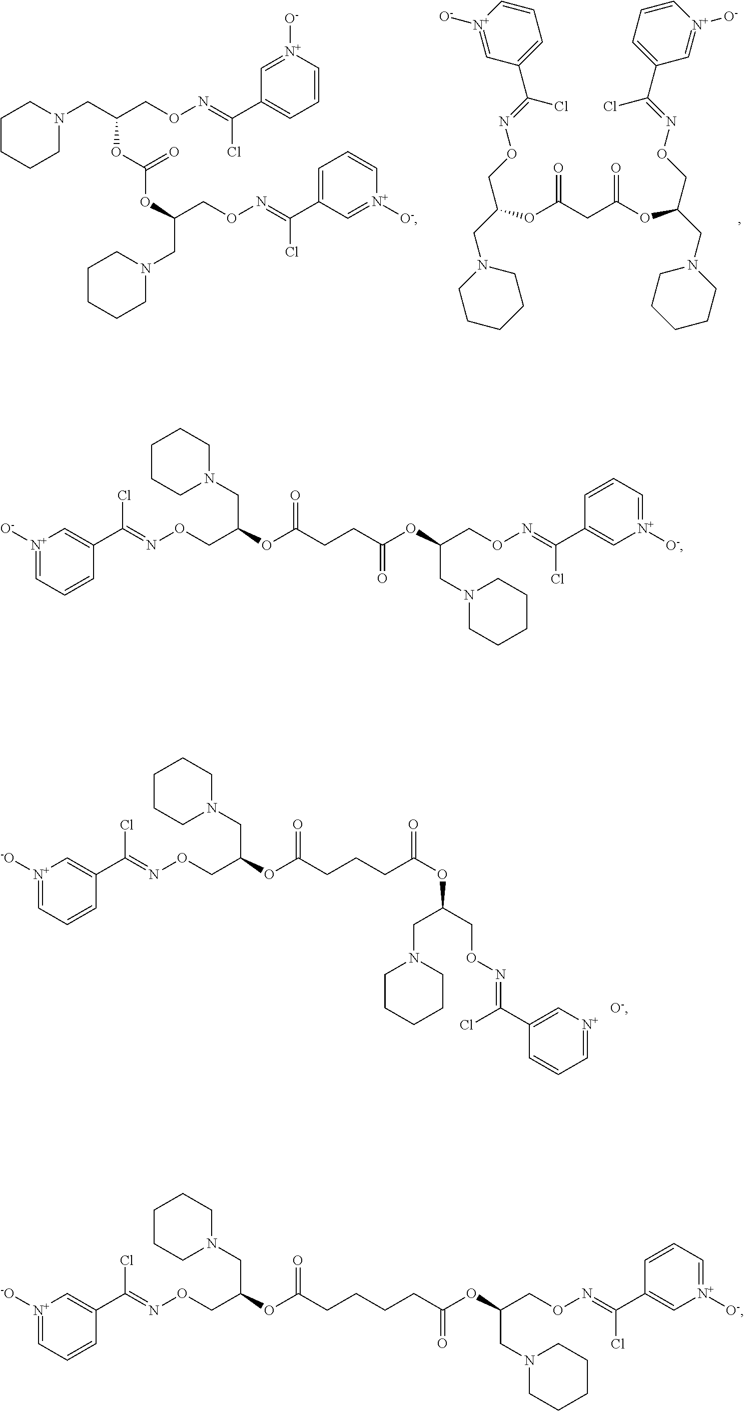

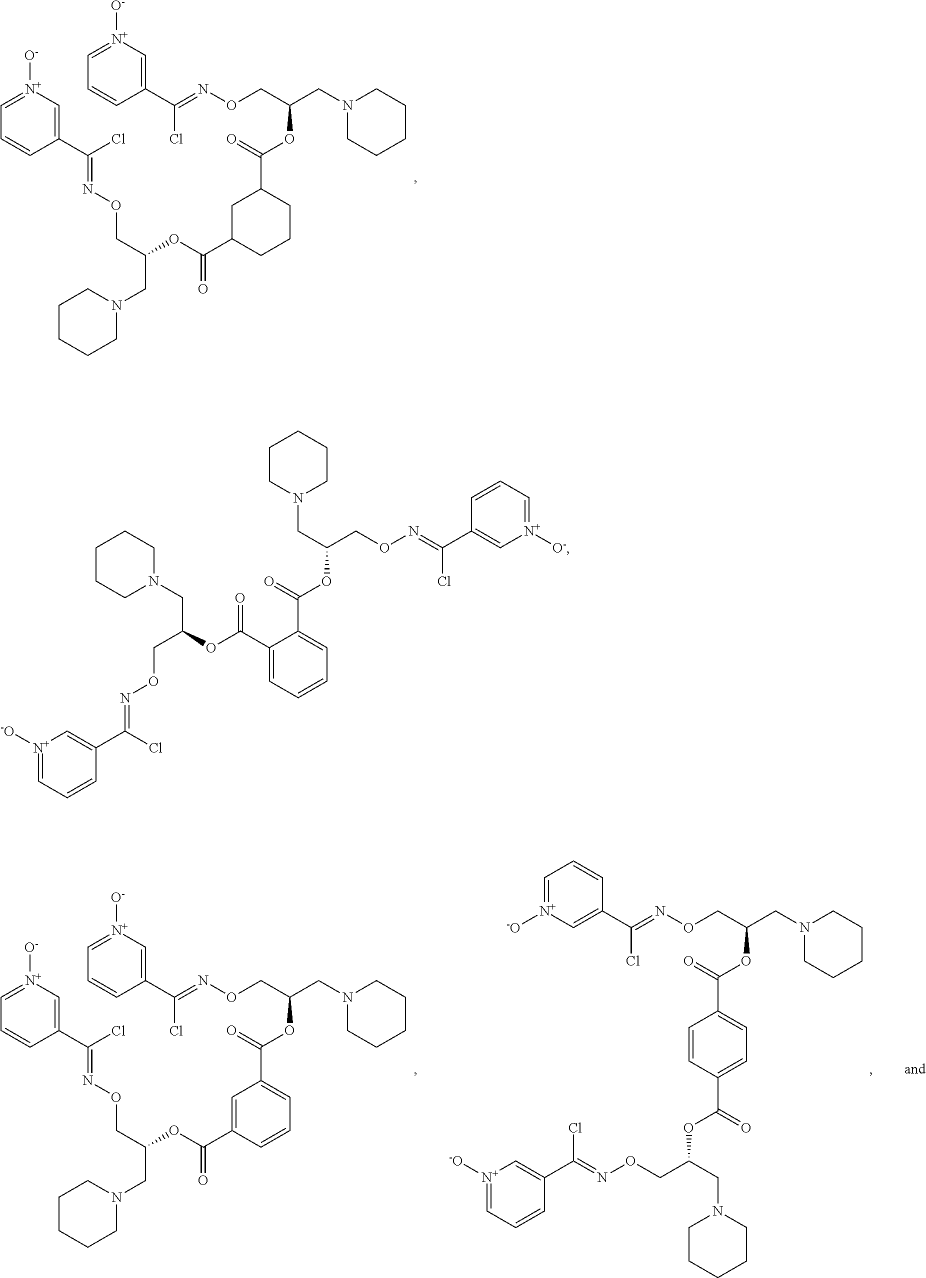

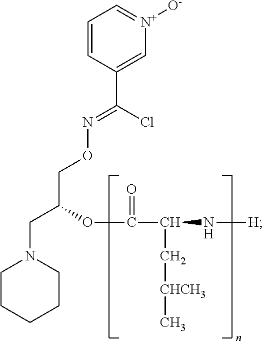

wherein n is 1-50;
or a pharmaceutically acceptable salt thereof.

In a still further embodiment, the present disclosure provides the compound of Formula IC, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In a further embodiment, the present disclosure provides a compound of Formula I, wherein the compound of Formula ID:

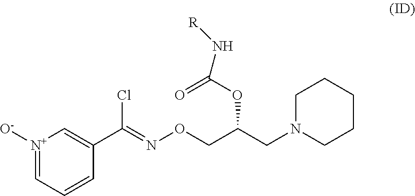

wherein R is selected from the group consisting of alkenyl, alkenyl, aminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a still further embodiment, the present disclosure provides a compound of Formula ID, wherein the compound of ID is a compound having a structure selected from:

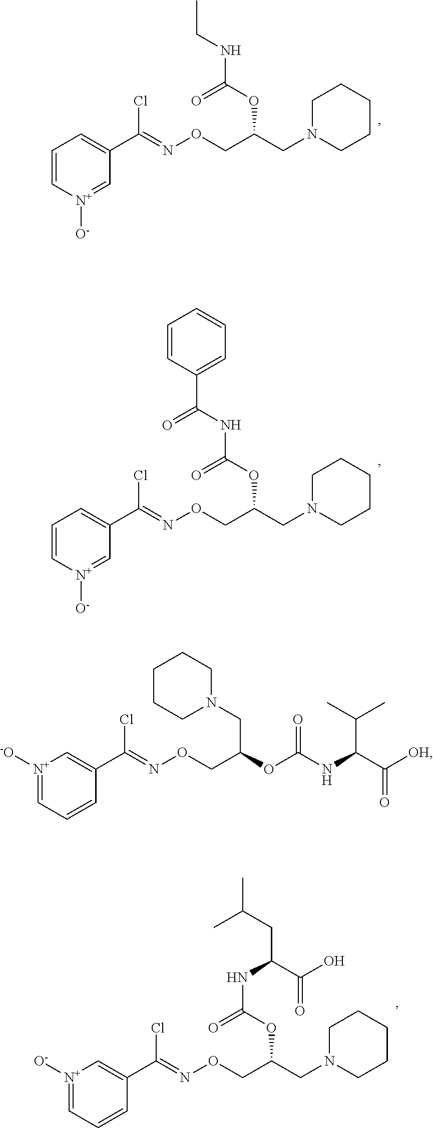

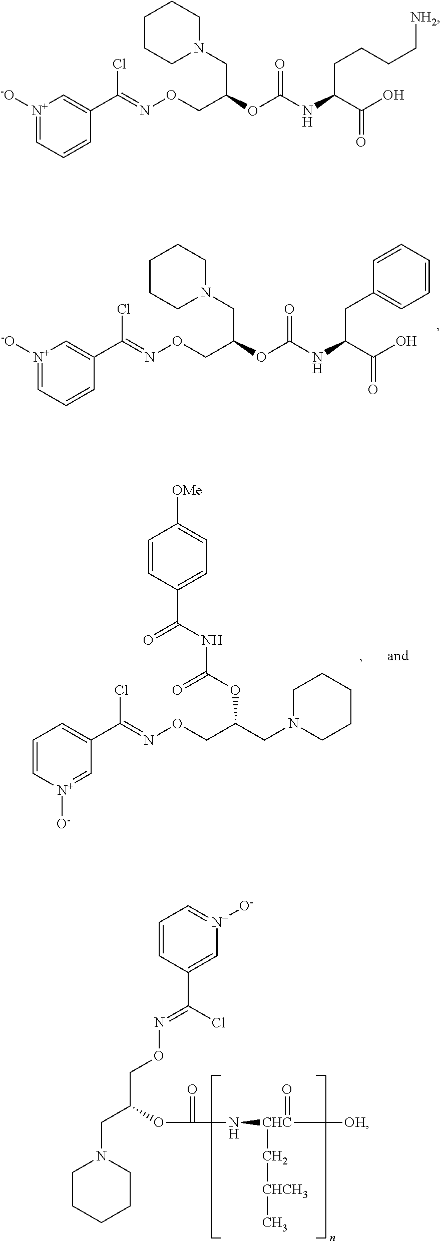

wherein n is 1-50;
or a pharmaceutically acceptable salt thereof.

In a still further embodiment, the present disclosure provides a compound of Formula ID, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In an embodiment, the present disclosure provides a compound of Formula II:

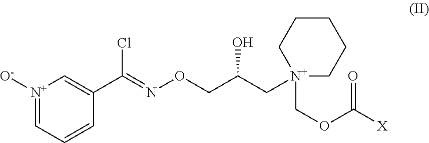

wherein R¹ and R² are independently selected from alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a further embodiment, the present disclosure provides a compound of Formula II, wherein the compound of Formula II has a structure selected from:

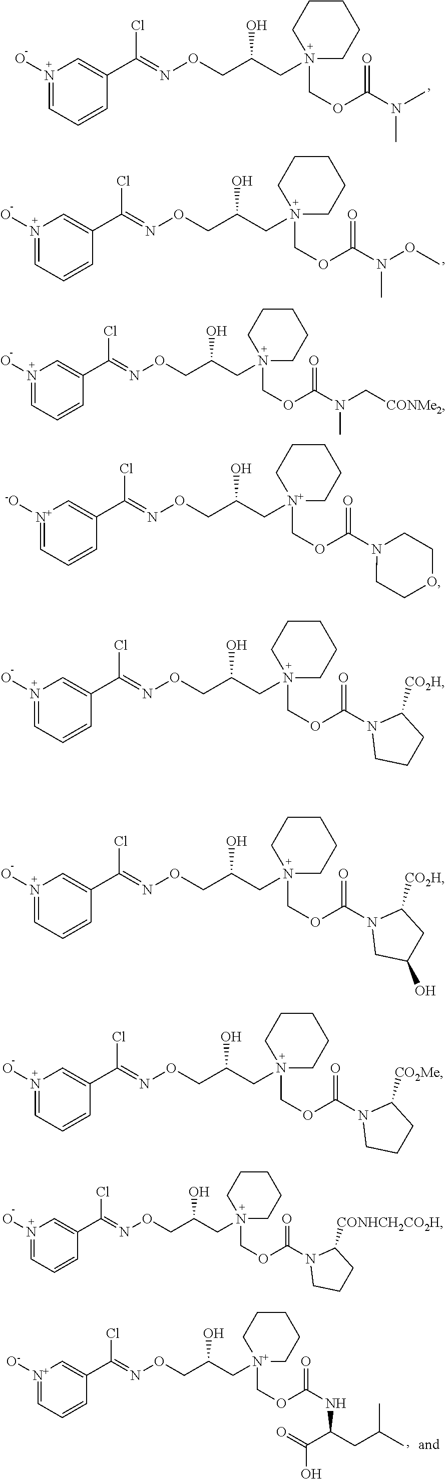

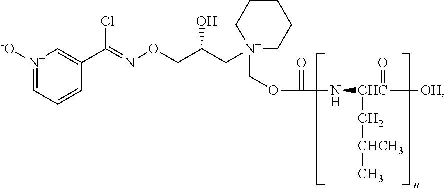

wherein n is 1-50;
or a pharmaceutically acceptable salt thereof.

In a still further embodiment, the present disclosure provides a compound of Formula IA, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50

In an embodiment, the present disclosure provides a compound of Formula III:

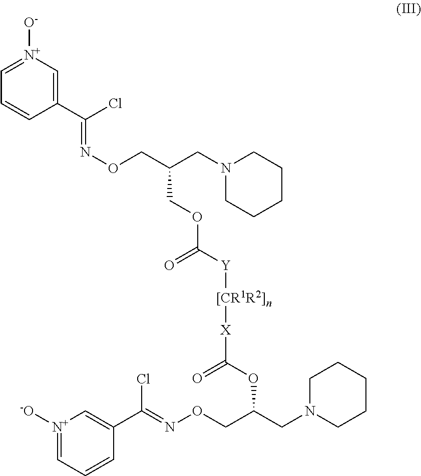

wherein X is selected from the group consisting of O, N, or absent,
Y is selected from the group consisting of O, N, or absent,
n is 1-10,
R¹ and R² are selected independently from each other and for each CR¹R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a further embodiment, the present disclosure provides a compound of Formula III, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In an embodiment, the present disclosure provides a compound of Formula IV:

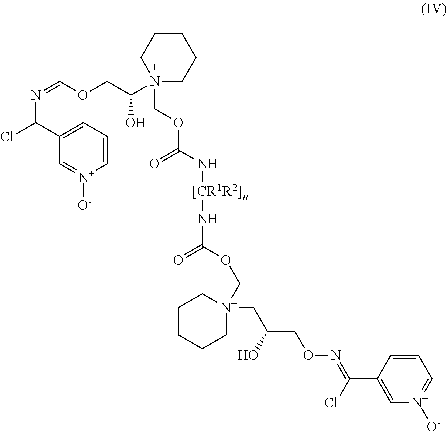

n is 1-10,
R¹ and R² are selected independently from each other and for each CR₁R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a further embodiment, the present disclosure provides compound of Formula IV, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In an embodiment, the present disclosure provides a compound of Formula V

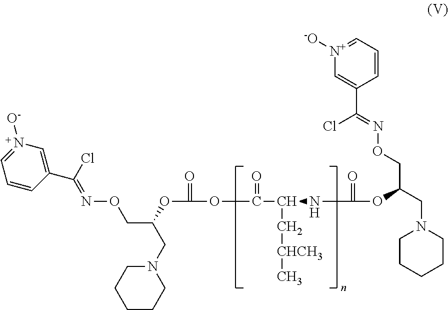

wherein n is 1-50,
or a pharmaceutically acceptable salt thereof.

In a further embodiment, the present disclosure provides a compound of Formula V, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In an embodiment, the present disclosure provides a compound of Formula VI:

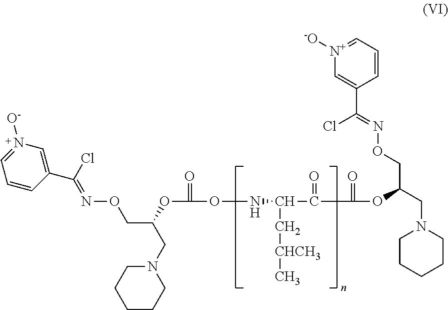

wherein n is 1-50;
or a pharmaceutically acceptable salt thereof.

In a further embodiment, the present disclosure provides a compound of Formula VI, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In an embodiment, the present disclosure provides a compound of Formula VII:

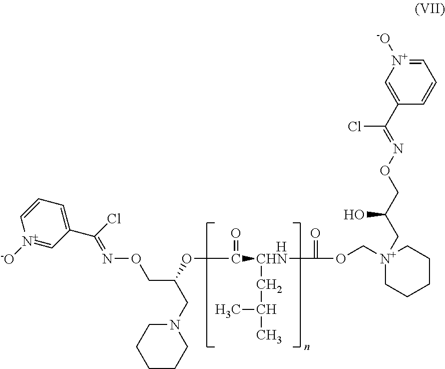

wherein n is 1-50,
or a pharmaceutically acceptable salt thereof.

In certain embodiment, the present disclosure provides a compound of Formula I to Formula VII, wherein the pharmaceutically acceptable salt is selected from sulfate, citrate, acetate, oxalate, chloride, hydrochloride, bromide, hydrobromide, iodide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate. In a still further embodiment, the pharmaceutically acceptable salt is citrate.

In an embodiment, the present disclosure provides a compound having a structure selected from:

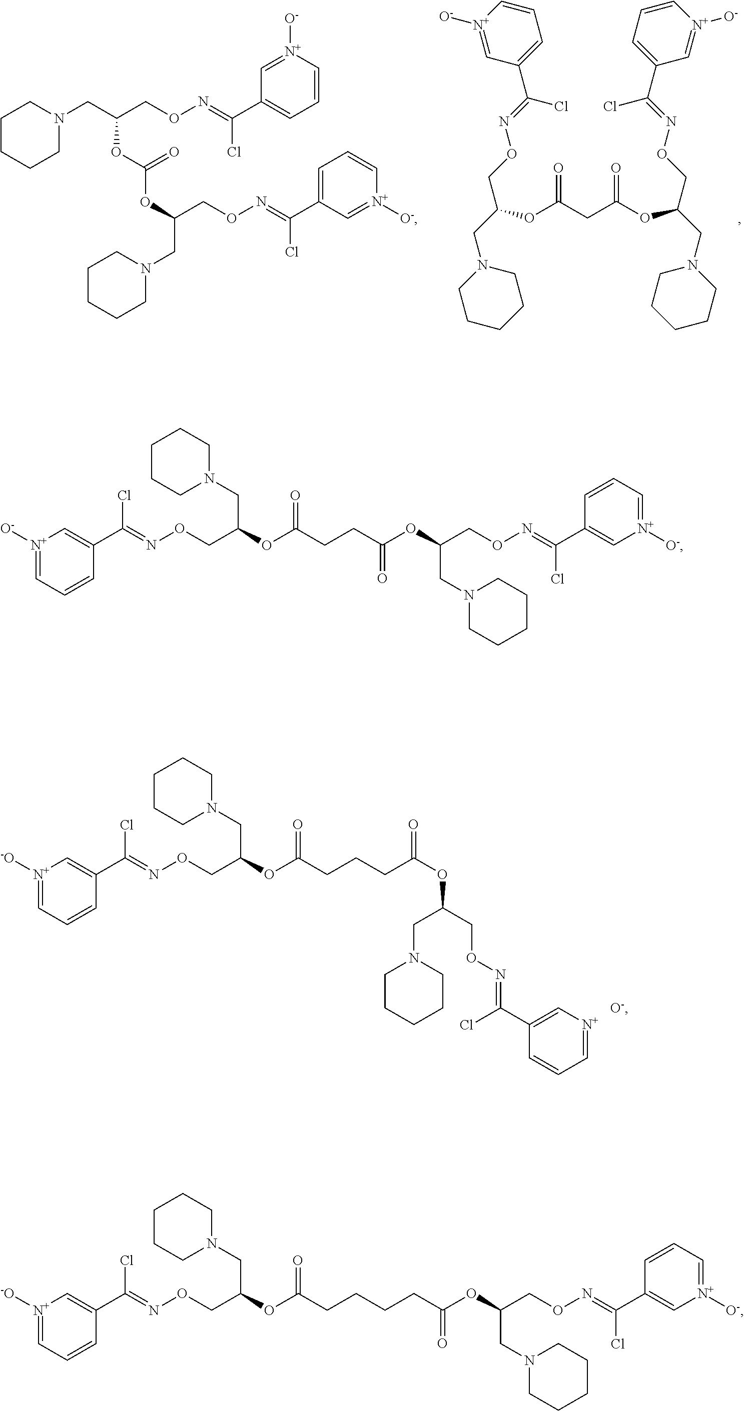

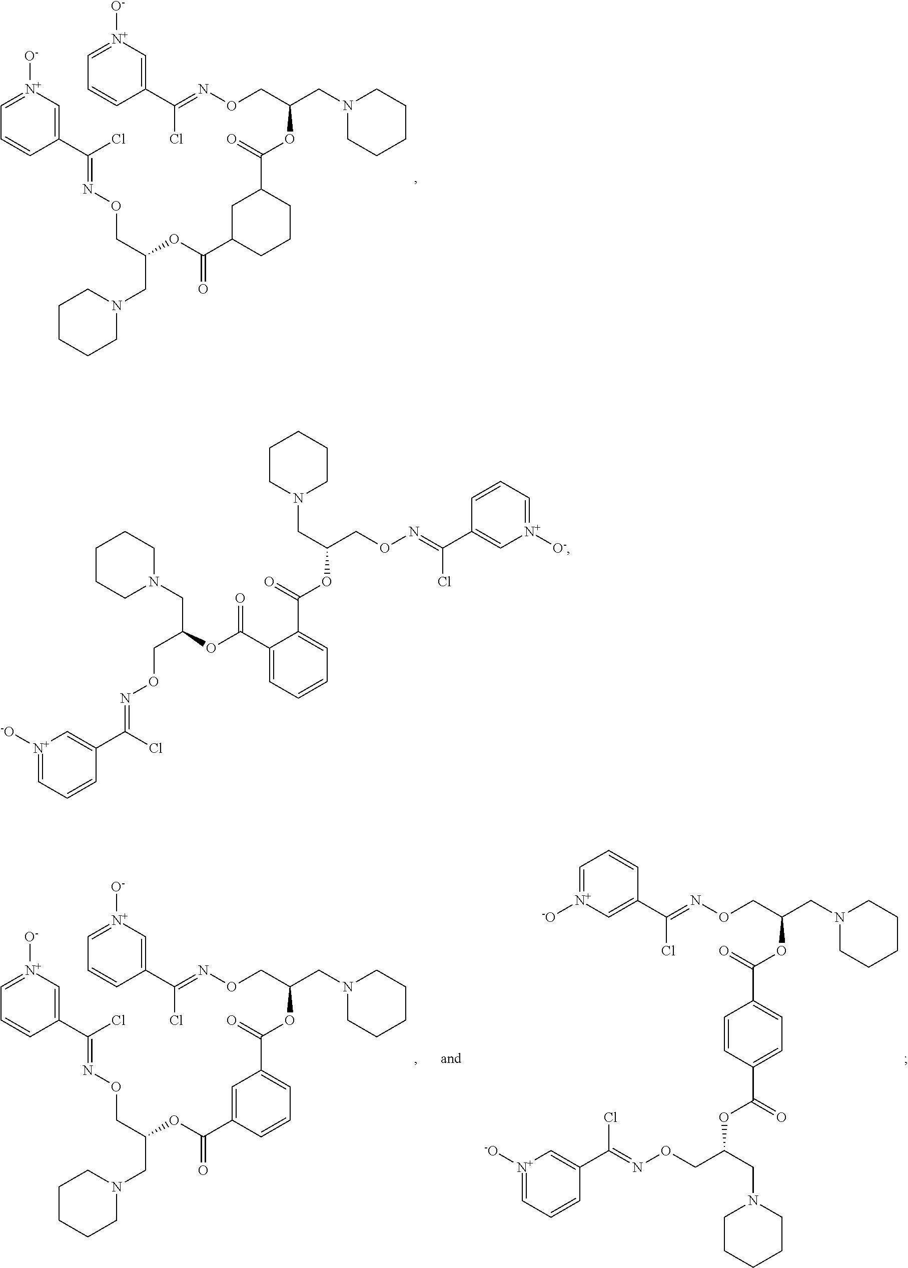

or a pharmaceutically acceptable salt thereof.

In another embodiment, the present disclosure provides a compound having a structure selected from

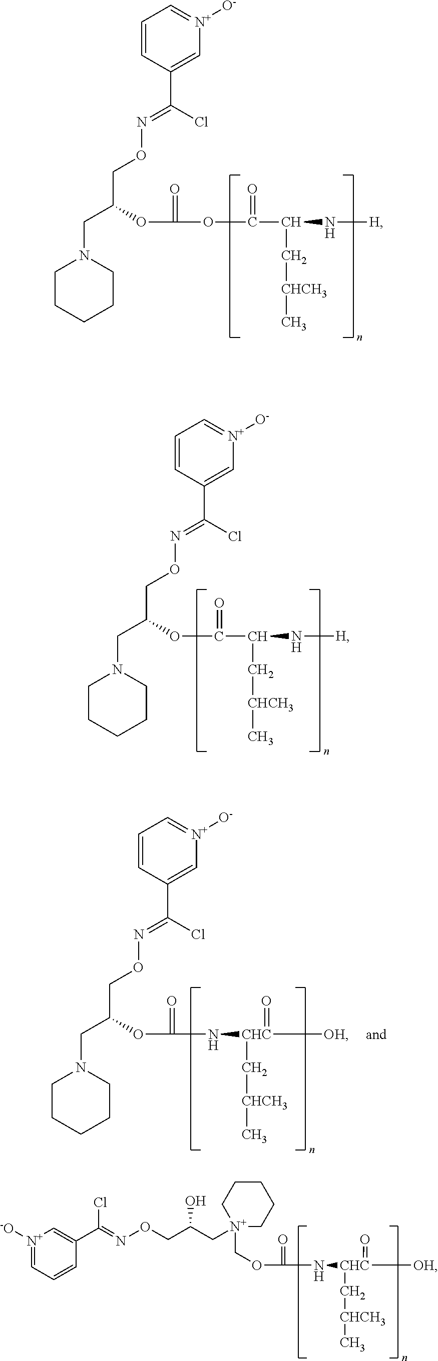

wherein n is 1-50; or a pharmaceutically acceptable salt thereof. Alternatively, n is 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

### Synthesis of Hydroxylamine Compounds

The compounds discussed above are generally synthesized by joining the free base form of arimoclomol to the X group of Formula I via a carbonyl group and to the X group of Formula II via an acyloxy methyl group. In some embodiments, compounds of Formula I and II are synthesized according to the following schemes.

### Synthesis of Arimoclomol Free Base 1:

To a suspension of arimoclomol citrate (5.0 g) in DCM (150 mL) was slowly added 5% aqueous NaHCO₃ (150 mL) at room temperature and stirred at room temperature for 1 h. The mixture was washed with DCM (2×50 mL). The combined DCM parts were washed with brine (100 mL), dried over anhydrous Na₂SO₄ and evaporated to dryness to give free base 1 (2.4 g, 78%)

### Synthesis of Ester Conjugates of Arimoclomol:

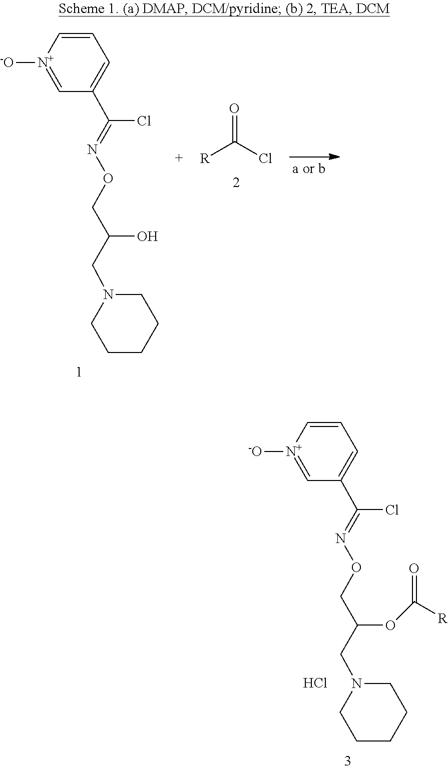

Compounds synthesized according Scheme 1 are shown in Table 1:

TABLE 1
Ester conjugates of arimoclomol.

| Compound | Structure | Name |
|---|---|---|
| 3a | 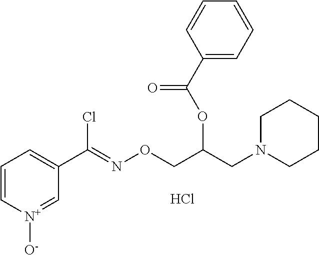 [SMILES: O=C(OC(CO/N=C(\Cl)c1ccc[n+]([O-])c1)CN1CCCCC1)c1ccccc1 ; Cl] | benzoyl-ARIM HCl |
| 3b | 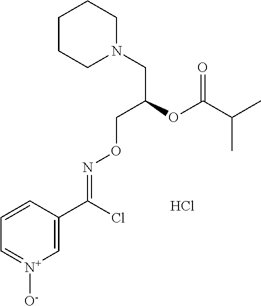 [SMILES: CC(C)C(=O)O[C@@H](CO/N=C(\Cl)c1ccc[n+]([O-])c1)CN1CCCCC1 ; Cl] | isobutyryl-ARIM HCl |
| 3c | 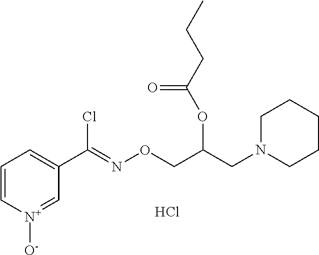 [SMILES: CCCC(=O)OC(CO/N=C(\Cl)c1ccc[n+]([O-])c1)CN1CCCCC1 ; Cl] | butyryl-ARIM HCl |
| 3d | 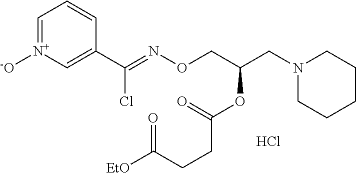 | ethyloxysuccinyl-ARIM HCl |
| 3e | 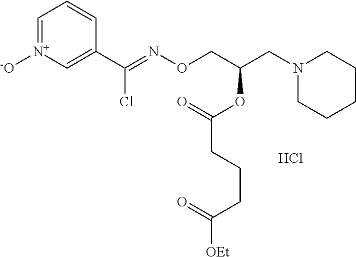 | ethyloxyglutaryl-ARIM HCl |
| 3f | 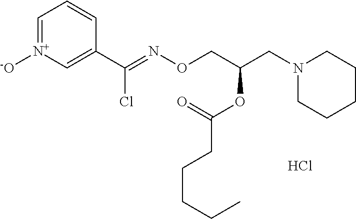 | hexanoyl-ARIM HCl |
| 3g | 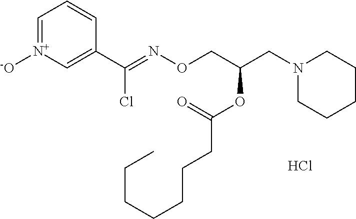 | octanoyl-ARIM HCl |
| 3h | 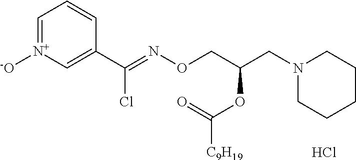 | decanoyl-ARIM HCl |
| 3i | 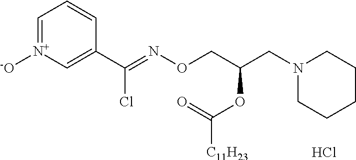 | dodecanoyl-ARIM HCl |
| 3j | 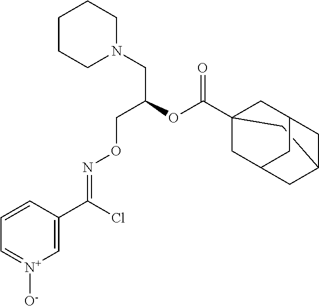 [SMILES: O=C(O[C@@H](CO/N=C(\Cl)c1ccc[n+]([O-])c1)CN1CCCCC1)C12CC3CC(CC(C3)C1)C2] | adamantly-CO-ARIM |

Synthesis of Ester Conjugates of Arimoclomol from Acid Chlorides 3a-j:

Method A (benzoate ester 3a): A solution of benzoyl chloride (0.16 mL, 1.35 mmol) in DCM (1 mL) was added dropwise to a solution of 1 (0.17 g, 0.54 mmol) and DMAP (0.03 g) in DCM/pyridine (8 mL, 3:1) at 0° C. After the addition, the reaction mixture was stirred overnight at room temperature. The reaction was quenched with a few drops of water and solvents were evaporated under reduced pressure. The residue was dissolved in EtOAc (80 mL) and washed with 5% aq. NaHCO₃ (40 mL) and brine (40 mL). The organic part was dried over Na₂SO₄ and evaporated to dryness. The crude product was purified by preparative HPLC.

The purified ester was dissolved in 1,4-dioxane (8 mL) and to the solution was added 4N HCl in dioxane (4 mL). The mixture was stirred in the room temp for 30 mins. The solvent was evaporated under reduced pressure. The residue was co-evaporated with IPAC and dried to give 3a as white solid (0.2 g, 81%)

Method B: To a solution of arimoclomol free base (1 eq) in DCM (10 mL/mmol) was added TEA (5 eq) and the mixture was cooled down to 0-5° C. A solution of acid chloride (2-2.5 eq) in DCM was added dropwise to the reaction mixture. After the addition, the reaction mixture was slowly brought to room temperature and then stirred for 6 h or overnight. The reaction mixture was diluted with DCM (50 mL), washed with 5% aq. NaHCO₃ (40 mL) and brine (40 mL), dried over anhydrous Na₂SO₄, and evaporated to dryness to afford the ester conjugate. The crude product was purified by preparative HPLC

The purified ester conjugate was dissolved in CH₃CN (4 mL) and to the solution was added 4N HCl/dioxane (2 mL). The reaction mixture was stirred at room temperature for 30 min., and solvent was evaporated. The resulting residue was co-evaporated with IPAc and dried to give 3b-i.

Synthesis of Ester Conjugates of Arimoclomol from Acids 5a-s:

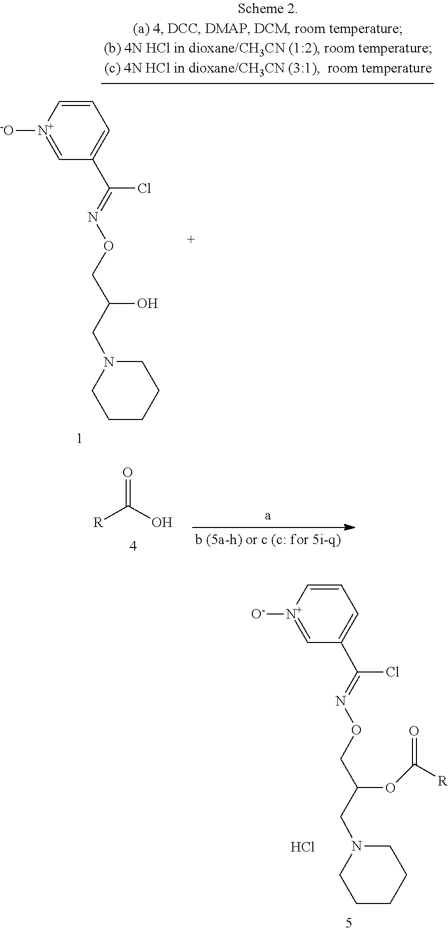

Compounds synthesized according to Scheme 2 are shown in Table 2:

TABLE 2
Ester conjugates of arimoclomol.

| Compound | Structure | Name |
|---|---|---|
| 5a | 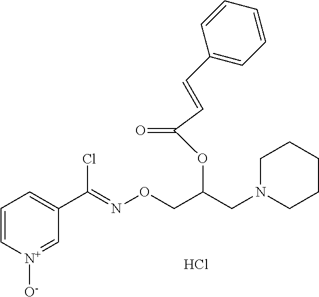 [SMILES: O=C(/C=C/c1ccccc1)OC(CO/N=C(\Cl)c1ccc[n+]([O-])c1)CN1CCCCC1 ; Cl] | cinnamoyl-ARIM HCl |
| 5b | 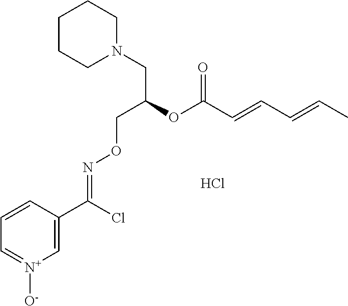 [SMILES: C/C=C/C=C/C(=O)O[C@@H](CO/N=C(\Cl)c1ccc[n+]([O-])c1)CN1CCCCC1 ; Cl] | sorboyl-ARIM HCl |
| 5c | 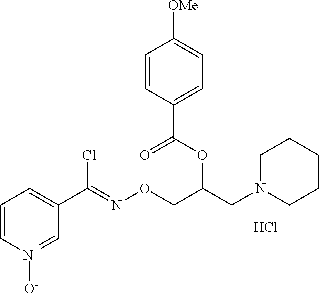 [SMILES: COc1ccc(C(=O)OC(CO/N=C(\Cl)c2ccc[n+]([O-])c2)CN2CCCCC2)cc1 ; Cl] | anisoyl-ARIM HCl |
| 5d | 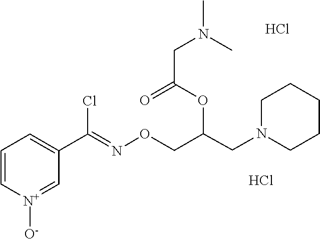 [SMILES: CN(C)CC(=O)OC(CO/N=C(\Cl)c1ccc[n+]([O-])c1)CN1CCCCC1 ; Cl ; Cl] | N,N-dimethylglycyl-ARIM 2 HCl |
| 5e | 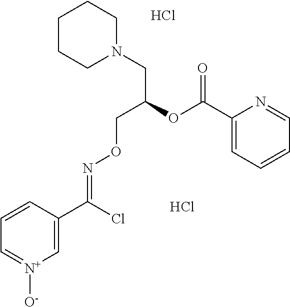 [SMILES: O=C(O[C@@H](CO/N=C(\Cl)c1ccc[n+]([O-])c1)CN1CCCCC1)c1ccccn1 ; Cl ; Cl] | picolinoyl-ARIM 2 HCl |
| 5f | 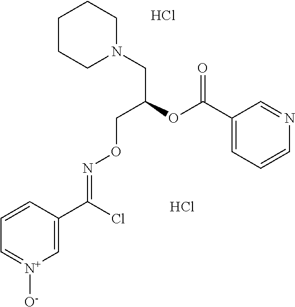 [SMILES: O=C(O[C@@H](CO/N=C(\Cl)c1ccc[n+]([O-])c1)CN1CCCCC1)c1cccnc1 ; Cl ; Cl] | nicotinoyl-ARIM 2 HCl |
| 5g | 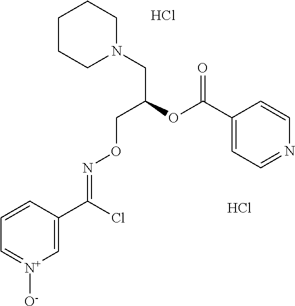 [SMILES: O=C(O[C@@H](CO/N=C(\Cl)c1ccc[n+]([O-])c1)CN1CCCCC1)c1ccncc1 ; Cl ; Cl] | isonicotinoyl-ARIM 2 HCl |
| 5h | 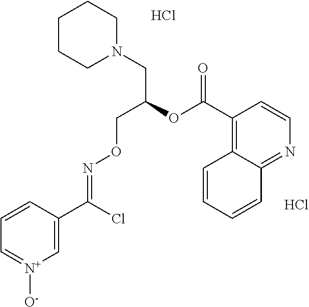 [SMILES: O=C(O[C@@H](CO/N=C(\Cl)c1ccc[n+]([O-])c1)CN1CCCCC1)c1ccnc2ccccc12 ; Cl ; Cl] | cinchoninoyl-ARIM 2 HCl |
| 5i | 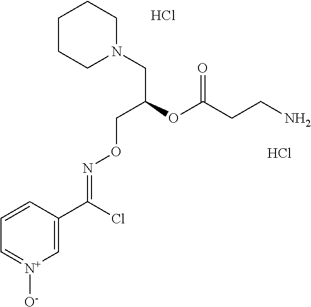 [SMILES: NCCC(=O)O[C@@H](CO/N=C(\Cl)c1ccc[n+]([O-])c1)CN1CCCCC1 ; Cl ; Cl] | β-Ala-ARIM 2 HCl |
| 5j | 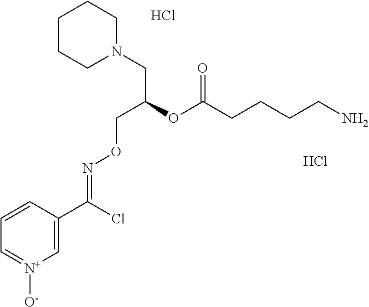 [SMILES: NCCCCC(=O)O[C@@H](CO/N=C(\Cl)c1ccc[n+]([O-])c1)CN1CCCCC1 ; Cl ; Cl] | 5-aminopentanoyl-ARIM 2 HCl |
| 5k | 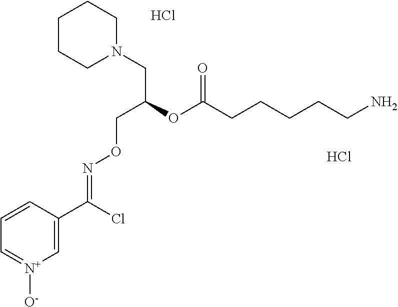 [SMILES: NCCCCCC(=O)O[C@@H](CO/N=C(\Cl)c1ccc[n+]([O-])c1)CN1CCCCC1 ; Cl ; Cl] | 6-aminohexanoyl-ARIM 2 HCl |
| 5l | 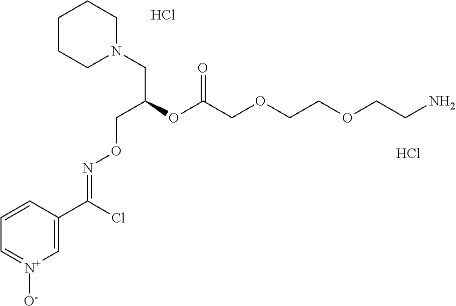 [SMILES: NCCOCCOCC(=O)O[C@@H](CO/N=C(\Cl)c1ccc[n+]([O-])c1)CN1CCCCC1 ; Cl ; Cl] | amino-PEG2-acetyl-ARIM 2 HCl |
| 5m | 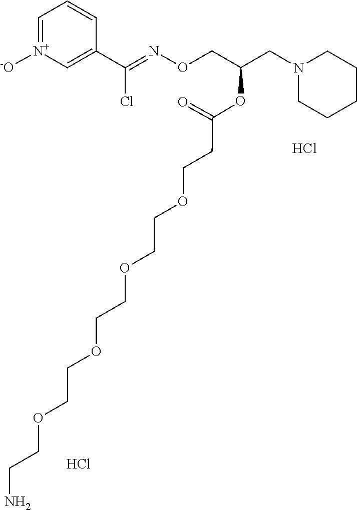 | amino-PEG4-propanoyl-ARIM 2 HCl |
| 5n | 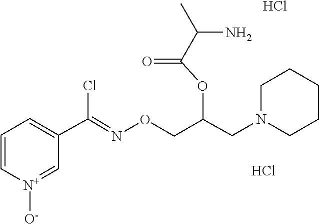 [SMILES: CC(N)C(=O)OC(CO/N=C(\Cl)c1ccc[n+]([O-])c1)CN1CCCCC1 ; Cl ; Cl] | Ala-ARIM 2 HCl |
| 50 | 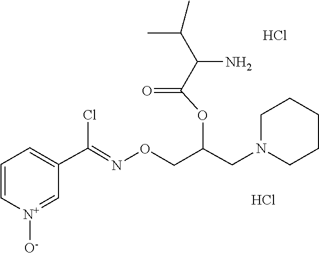 [SMILES: CC(C)C(N)C(=O)OC(CO/N=C(\Cl)c1ccc[n+]([O-])c1)CN1CCCCC1 ; Cl ; Cl] | Val-ARIM 2 HCl |
| 5p | 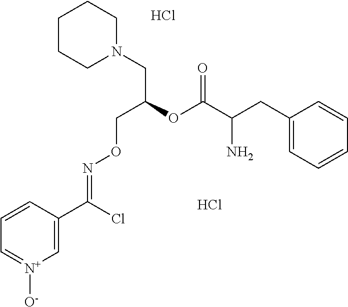 [SMILES: NC(Cc1ccccc1)C(=O)O[C@@H](CO/N=C(\Cl)c1ccc[n+]([O-])c1)CN1CCCCC1 ; Cl ; Cl] | Phe-ARIM 2 HCl |
| 5q | 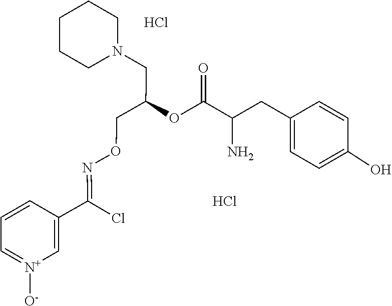 [SMILES: NC(Cc1ccc(O)cc1)C(=O)O[C@@H](CO/N=C(\Cl)c1ccc[n+]([O-])c1)CN1CCCCC1 ; Cl ; Cl] | Tye-ARIM 2 H |
| 5r | 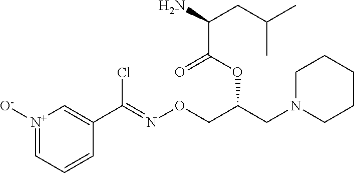 [SMILES: CC(C)C[C@H](N)C(=O)O[C@@H](CO/N=C(\Cl)c1ccc[n+]([O-])c1)CN1CCCCC1] | Ile-ARIM 2 HCl |
| 5s | 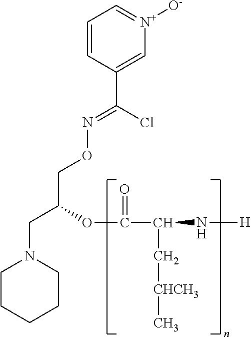 | Leuₙ-ARIM |

To a solution of arimoclomol free base 1 (1 mmol), acid/protected amino acid (1.1-1.2 mmol) and DMAP (0.1 mmol) in DCM (10 mL) was added a solution of DCC (1.1-1.2 mmol) dropwise at room temperature. The reaction mixture was stirred at room temperature overnight and the suspension was filtered. The filtrate was evaporated to dryness and the crude product was purified by preparative HPLC to give the ester conjugate 5.

The purified ester was dissolved in CH₃CN or 1,4-dioxane (6 mL), and 4N HCl/dioxane (3 mL) was added. The reaction mixture was stirred at room temperature for 1 h. Solvent was evaporated under reduced pressure. The resulting residue was co-evaporated with IPAC and dried to give the corresponding hydrochloride salt 5a-h.

To a solution of Boc-protected amino acid ester in CH₃CN (3 mL) was added 4N HCl/dioxane (9 mL) and the reaction mixture was stirred at room temperature for 1-3 h. Solvent was evaporated under reduced pressure. The residue was co-evaporated with IPAC and dried to give amino acid esters Si-s.

### Synthesis of Monoester of Dicarboxylic Acid 7a-e:

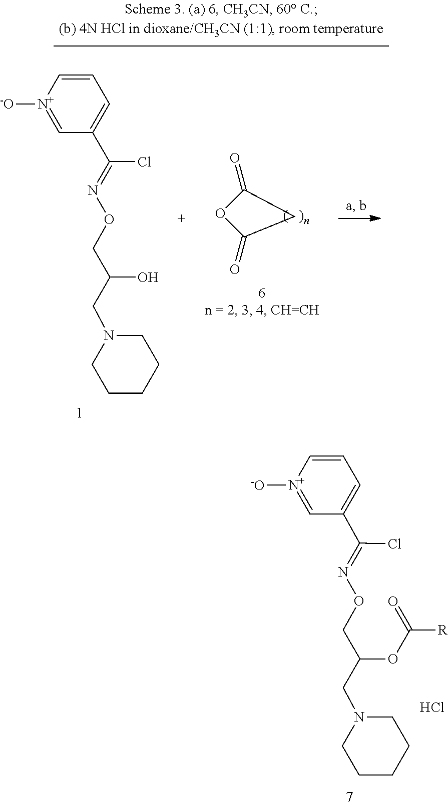

Compounds synthesized according to Scheme 3 are shown in Table 3:

TABLE 3
Mono-ester conjugates of dicarboxylic acids

| Compound | Structure | Name |
|---|---|---|
| 7a | 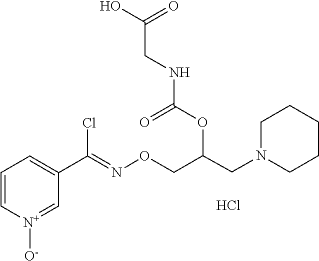 [SMILES: O=C(O)CNC(=O)OC(CO/N=C(\Cl)c1ccc[n+]([O-])c1)CN1CCCCC1 ; Cl] | cinnamoyl-ARIM HCl |
| 7b | 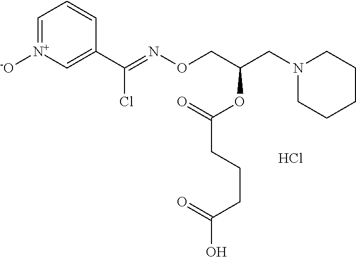 | sorboyl-ARIM HCl |
| 7c | 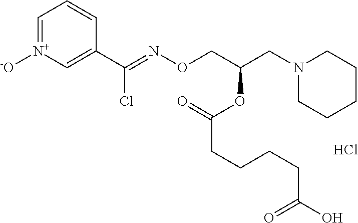 | anisoyl-ARIM HCl |
| 7d | 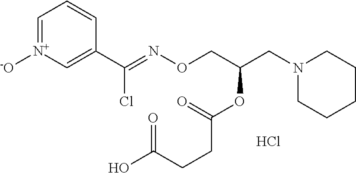 | N,N-dimethylglycyl-ARIM 2 HCl |
| 7e | 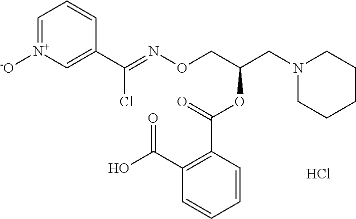 | picolinoyl-ARIM 2 HCl |

A solution of arimoclomol free base 1 (1 eq) and acid anhydride 6 (1.2 eq) in CH₃CN was heated at 50° C. for 1-2 h. The solvent evaporated under reduced pressure and the residue dried under vacuum. The product was redissolved in CH₃CN (5 mL) and 4N HCl/dioxane (5 mL) was added. After stirring at the room temperature for 30 min., solvent was evaporated. The residue was co-evaporated with IPAC and dried to give 7a-e as white solid.

Synthesis of Carbonate conjugates of Arimoclomol 9a-e:

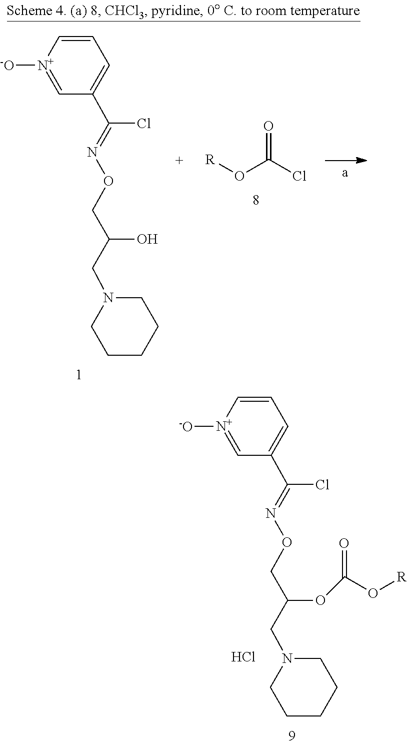

Compounds synthesized according to Scheme 4 are shown in Table 4:

TABLE 4
Carbonate prodrugs of arimoclomol

| Compound | Structure | Name |
|---|---|---|
| 9a | 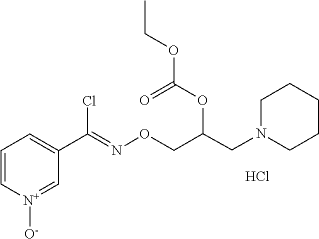 [SMILES: CCOC(=O)OC(CO/N=C(\Cl)c1ccc[n+]([O-])c1)CN1CCCCC1 ; Cl] | ethyl-O-CO-ARIM HCl |
| 9b | 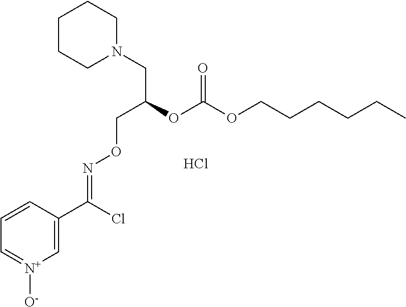 [SMILES: CCCCCCOC(=O)O[C@@H](CO/N=C(\Cl)c1ccc[n+]([O-])c1)CN1CCCCC1 ; Cl] | hexyl-O-CO-ARIM HCl |
| 9c | 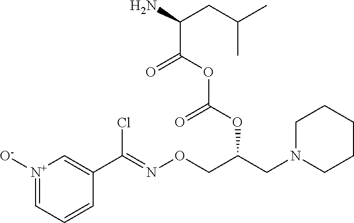 [SMILES: CC(C)C[C@H](N)C(=O)OC(=O)O[C@@H](CO/N=C(\Cl)c1ccc[n+]([O-])c1)CN1CCCCC1] | Leu-O-CO-AIM |
| 9d | 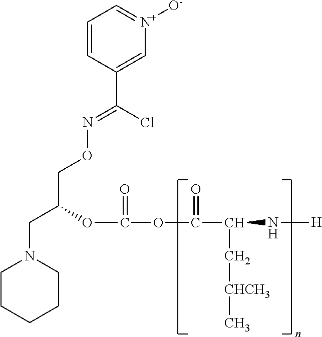 | Leuₙ-O-CO-ARIM |
| 9e | 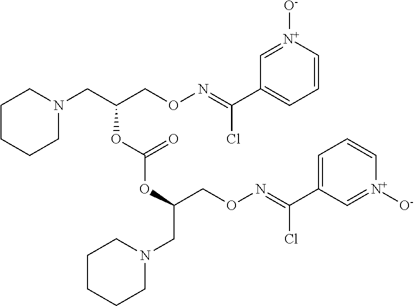 [SMILES: O=C(O[C@@H](CO/N=C(\Cl)c1ccc[n+]([O-])c1)CN1CCCCC1)O[C@@H](CO/N=C(\Cl)c1ccc[n+]([O-])c1)CN1CCCCC1] | ARIM-CO-ARIM |

A solution of alkyl chloroformate 8 in CHCl₃ (2 mL) was added dropwise to a solution of arimoclomol free base 1 (1 mmol) in CHCl₃ (8 mL) and pyridine (1 mL) at 0° C. The reaction mixture was stirred at 0-5° C. for 30 min. followed by 2 h at room temperature. The reaction was quenched with water and solvent was evaporated under reduced pressure. The residue was taken in EtOAc, washed with 5% aq. NaHCO₃ and brine, dried over Na₂SO₄, and evaporated to dryness. The crude product was purified by preparative HPLC to give the carbonate conjugate.

Pure carbonate was dissolved in CH₃CN (5 mL) and 4N HCl in dioxane (2.5 mL) was added to the solution. The mixture was stirred at room temperature for 30 min. Solvent was evaporated under reduced pressure. The resulting residue was co-evaporated with IPAc and dried to give compound 9a-e.

### Synthesis of Carbamate Conjugates of Arimoclomol 12a-f:

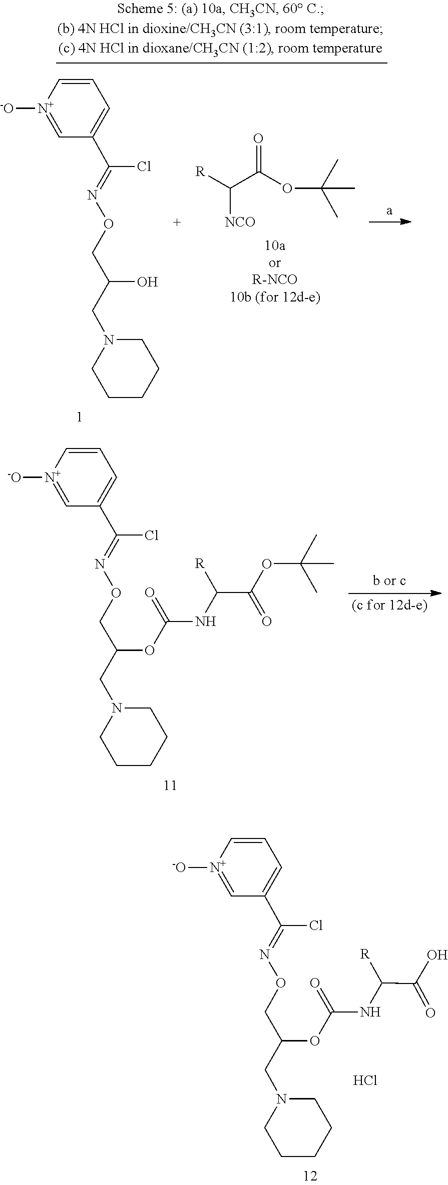

Compounds synthesized according to Scheme 5 are shown in Table 5:

TABLE 5
Carbamate prodrugs of arimoclomol

| Compound | Structure | Name |
|---|---|---|
| 12a | 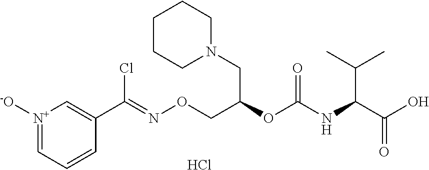 | ARIM-CO-Val HCl |
| 12b | 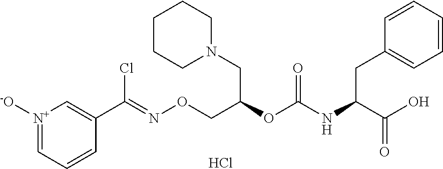 | ARIM-CO-Phe HCl |
| 12c | 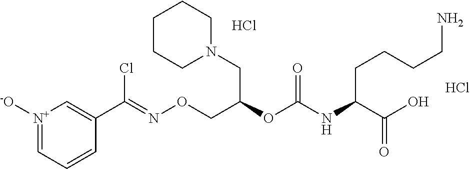 | ARIM-CO-Lys 2 HCl |
| 12d | 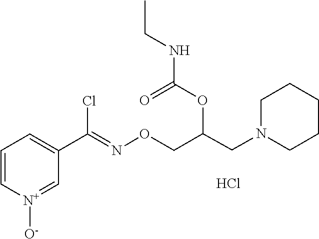 [SMILES: CCNC(=O)OC(CO/N=C(\Cl)c1ccc[n+]([O-])c1)CN1CCCCC1 ; Cl] | ethyl-NH—CO-ARIM HCl |
| 12e | 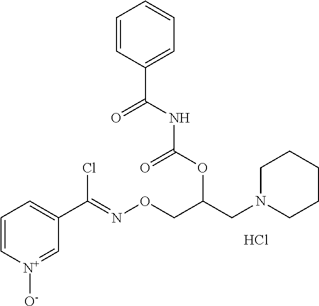 [SMILES: O=C(NC(=O)c1ccccc1)OC(CO/N=C(\Cl)c1ccc[n+]([O-])c1)CN1CCCCC1 ; Cl] | benzoyl-NH—CO-ARIM HCl |
| 12f | 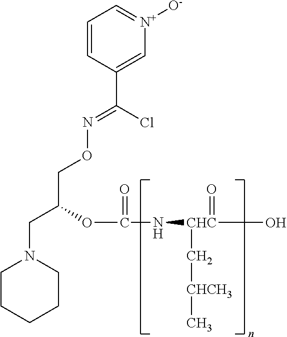 | ARIM-CO-Leuₙ |

A solution of arimoclomol free base 1 (1 eq) and isocyanate 10a or 10b (1.5 eq) in CH₃CN (5 ml-) was heated at 60° C. for 6-8 h. Solvent was evaporated, and the crude product was purified by preparative HPLC to afford 11a-d and 11f. Similarly, the reaction of arimoclomol free base 1 with benzoyl isocyanate (10b) in DCM at room temperature produced the corresponding carbamate 12e.

To a solution of 11a-c and 11f in 1,4-dioxane (3 ml-) was added 4N HCl/dioxane (9 ml-) and the reaction mixture was stirred overnight at room temperature. The solvent was evaporated under vacuum. The resulting residue was co-evaporated with IPAc and dried to give 12a-c and 12f.

### Synthesis of Bis-Arimoclomol Esters 14a-i:

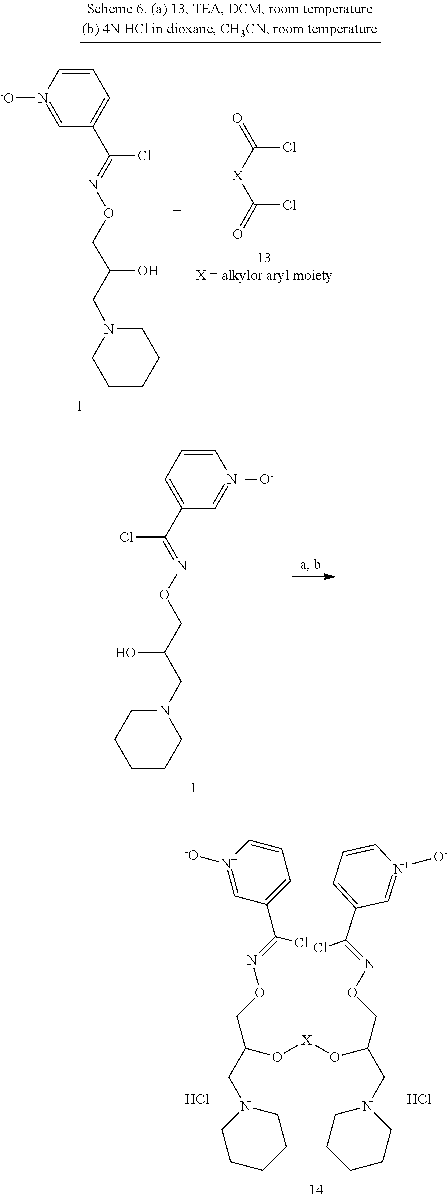

Compounds synthesized according to Scheme 6 are shown in Table 6:

TABLE 6
Bis-arimoclomol ester prodrugs

| Compound | Structure | Name |
|---|---|---|
| 14a | 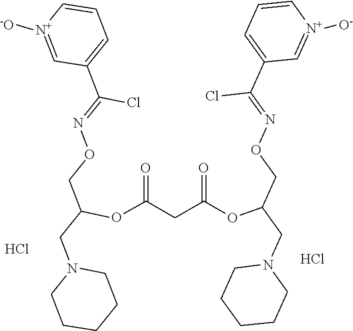 | ARIM-malonyl- ARIM 2 HCl |
| 14b | 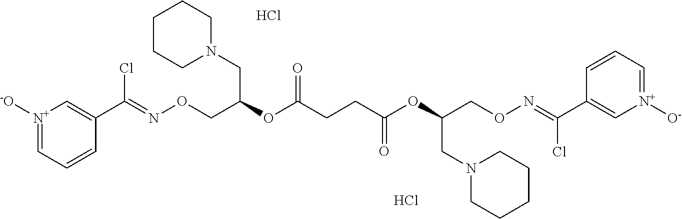 | ARIM-succinyl- ARIM 2 HCl |
| 14c | 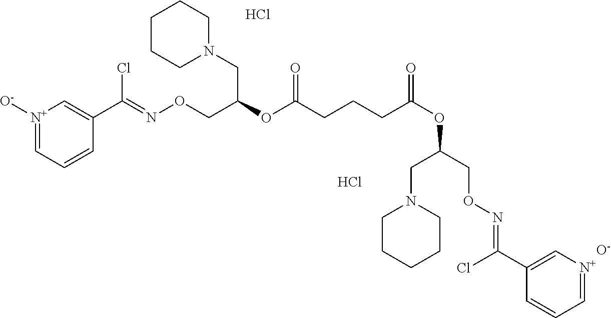 | ARIM-glutaryl- ARIM 2 HCl |
| 14d | 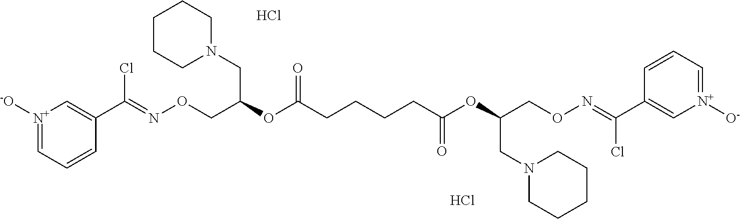 | ARIM-adipoyl- ARIM 2 HCl |
| 14e | 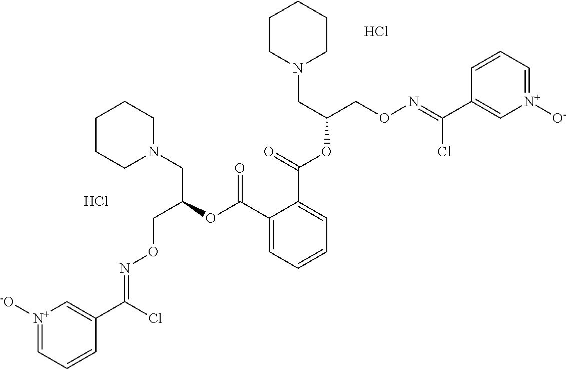 | ARIM- phthaloyl-ARIM 2 HCl |
| 14f | 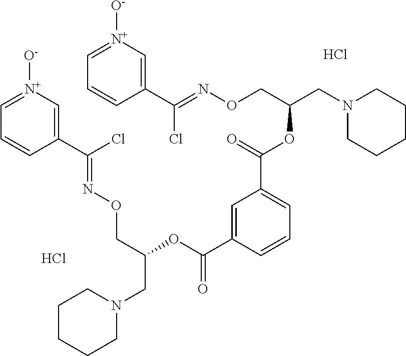 [SMILES: O=C(O[C@@H](CO/N=C(\Cl)c1ccc[n+]([O-])c1)CN1CCCCC1)c1cccc(C(=O)O[C@@H](CO/N=C(\Cl)c2ccc[n+]([O-])c2)CN2CCCCC2)c1 ; Cl ; Cl] | ARIM- isophthaloyl- ARIM 2 HCl |
| 14g | 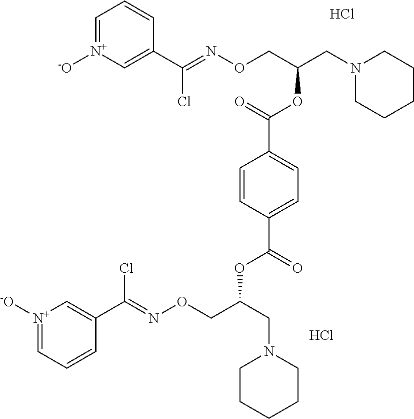 | ARIM- terephthaoyl- ARIM 2 HCl |
| 14h | 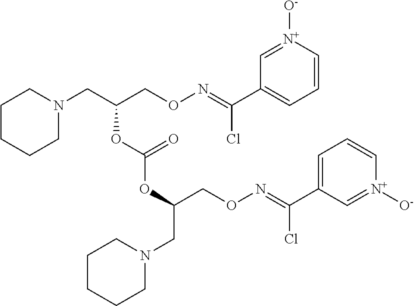 [SMILES: O=C(O[C@@H](CO/N=C(\Cl)c1ccc[n+]([O-])c1)CN1CCCCC1)O[C@@H](CO/N=C(\Cl)c1ccc[n+]([O-])c1)CN1CCCCC1] | ARIM-CO- ARIM |
| 14i | 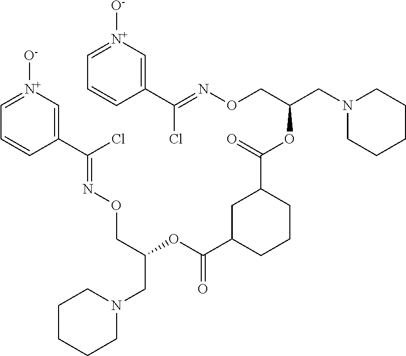 [SMILES: O=C(O[C@@H](CO/N=C(\Cl)c1ccc[n+]([O-])c1)CN1CCCCC1)C1CCCC(C(=O)O[C@@H](CO/N=C(\Cl)c2ccc[n+]([O-])c2)CN2CCCCC2)C1] | ARIM- (cyclohexane- 1,3-dicarbonyl)- ARIM |

To a solution of acid chloride 13 (2 mmol) in DCM (6 ml-) TEA (3 mmol) was added dropwise under inert condition. A solution of arimoclomol free base 1 (1 mmol) in DCM (4 ml-) was added dropwise. The reaction mixture was stirred overnight at room temperature, then solvent was removed under reduced pressure. The residue was dissolved in DCM (50 ml-) and washed with 5% aq. NaHCO₃ and brine, dried over Na₂SO₄, and evaporated to dryness. The crude product was purified by preparative HPLC to give the diester.

The purified diester was dissolved in CH₃CN (6 mL) and 4NHCl/dioxane (3 mL) was added. After stirring at room temperature for 30 min., solvent was evaporated under reduced pressure. The residue was co-evaporated with IPAC and dried to produce HCl salt 14.

Synthesis of Carboxymethyl Conjugates at Arimoclomol Piperidine N-10 (18a-i)

Prodrugs with carboxymethyl at the arimoclomol piperidine N-10 having the structure of Formula II are generally synthesized by joining arimoclomol freebase or optionally benzyl-protected (at C-8 hydroxyl) arimoclomol and a chloromethyl carbamate.

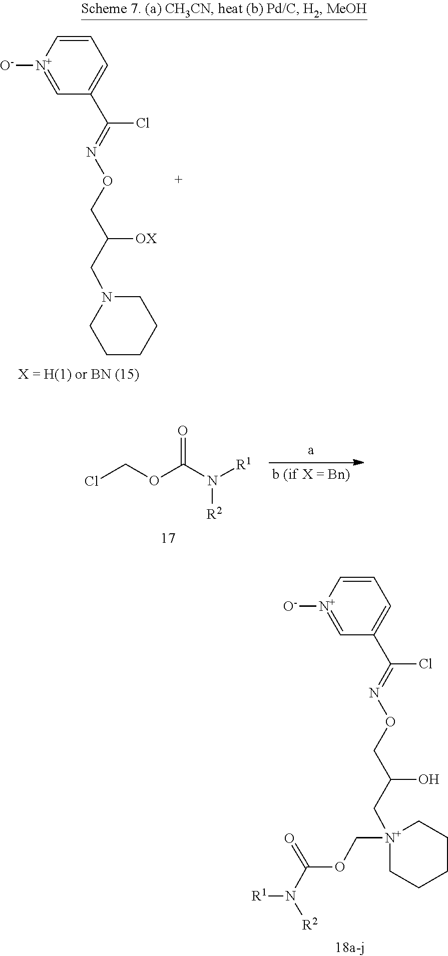

Compounds synthesized according to Scheme 7 are shown in Table 7:

TABLE 7
Prodrugs with carboxymethyl at arimoclomol piperidine N-10

| Compound | Structure | Name |
|---|---|---|
| 18a | 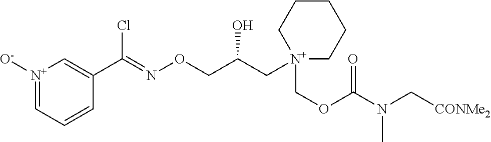 | N,N- dimethylacetamidomethyl- N(Me—CO—OCH₂-ARIM |
| 18b | 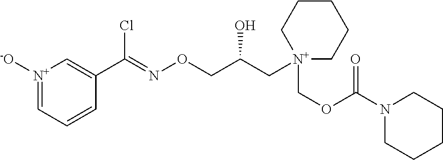 | N-morpholinyl-CO—OCH₂- ARIM |
| 18c | 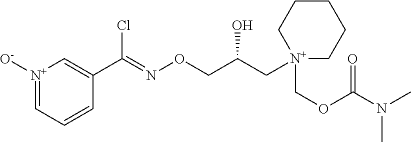 [SMILES: CN(C)C(=O)OC[N+]1(C[C@@H](O)CO/N=C(\Cl)c2ccc[n+]([O-])c2)CCCCC1] | methyl-N(Me)—CO—OCH₂- ARIM |
| 18d | 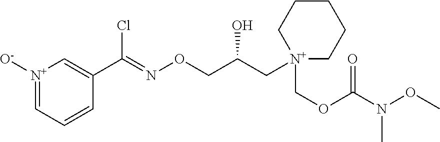 [SMILES: CON(C)C(=O)OC[N+]1(C[C@@H](O)CO/N=C(\Cl)c2ccc[n+]([O-])c2)CCCCC1] | N-methoxy-N(Me)—CO—OCH₂- ARIM |
| 18e | 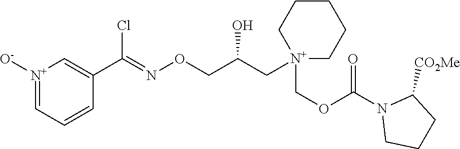 [SMILES: COC(=O)[C@@H]1CCCN1C(=O)OC[N+]1(C[C@@H](O)CO/N=C(\Cl)c2ccc[n+]([O-])c2)CCCCC1] | N-(2- methoxycarbonylpyrrolidinyl)- CO—OCH₂-ARIM |
| 18f | 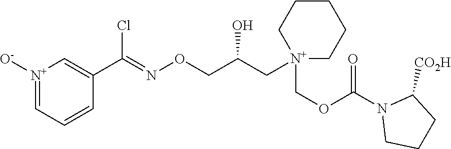 [SMILES: O=C(O)[C@@H]1CCCN1C(=O)OC[N+]1(C[C@@H](O)CO/N=C(\Cl)c2ccc[n+]([O-])c2)CCCCC1] | N-(2-carboxypyrrolidinyl)- CO—OCH₂-ARIM |
| 18g | 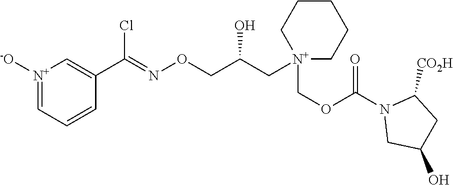 | N-(2-carboxy-4- hydroxypyrrolidinyl)-CO—OCH₂- ARIM |
| 18h | 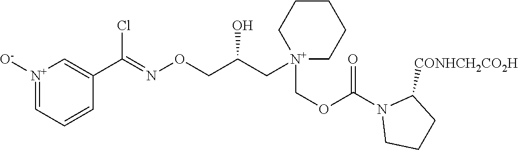 | N-(2- glycinylcarbonylpyrrolidinyl)- CO—OCH₂-ARIM |
| 18i | 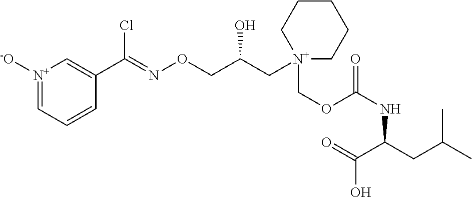 | ARIM-CH₂O—CO-Leu |
| 18j | 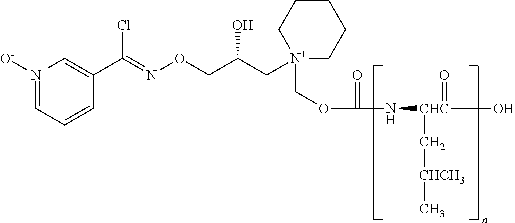 | ARIM-CH₂O—CO-Leuₙ |

A suspension of arimoclomol freebase in 80% acetic acid is heated at 80° C. until the reaction is complete. The reaction mixture is cooled to room temperature and solvent evaporated under reduced pressure. The resulting residue is dissolved in DCM and washed with 5% aq. NaHCO₃ solution and water. The organic phase is dried over anhydrous Na₂SO₄ and evaporated to dryness to produce compound 15. The crude product is purified by flash column chromatography.

### Synthesis of Chloromethyl Carbamate 17

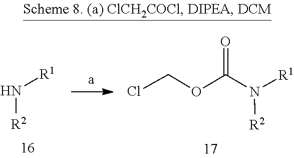

Compound 16 in DCM is cooled in an ice-water bath. DIPEA is added and the resulting mixture is stirred for 5 min. Chloromethyl chloroformate in DCM is added. The mixture is stirred at 0-10° C. until the reaction is complete. Subsequently, 5% of aq. NH₄Cl is added to quench the reaction. The DCM layer is dried over Na₂SO₄. The solvent is evaporated and the residue purified by silica gel chromatography.

### Synthesis of Carboxymethyl Conjugates at Arimoclomol N-10

To compound 1 or 15 in CH₃CN is added compound 17. The mixture is heated until the reaction is complete. Solvent is evaporated, and the crude product is purified by preparative HPLC to afford compound 18 (if starting material is 1) or protected 18 (if starting material is 15).

### Optional Deprotection of Protected Compound 18

To protected 18 in MeOH is added Pd/C (10% Pd, 100% wt/wt) and the mixture is stirred under hydrogen until the reaction is complete. The suspension is filtered through celite, washed with MeOH and the combined filtrates are evaporated under reduced pressure. The resulting residue is purified by preparative HPLC to give 18.

### II. Pharmaceutical Compositions

“Pharmaceutical composition” as it relates to the present technology means a composition comprising at least one active pharmaceutical ingredient (API) and, optionally, one or more excipients as defined herein.

In one embodiment, the present disclosure describes a composition comprising:
a therapeutically effective amount of a compound of Formula I:

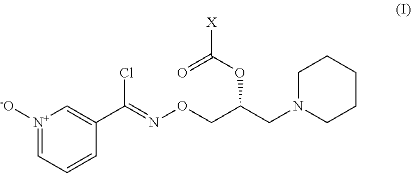

wherein X is selected from the group consisting of R, [O—R], [NR¹R²], and [NRH], where
O is oxygen, N is nitrogen, and H is hydrogen;
R, R¹ and R² are independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

“Pharmaceutically effective amount” as it relates to the present technology means an amount that has a pharmacological effect. “Pharmaceutically effective amount” may be used interchangeably with “therapeutically effective amount,” which as used herein, means an amount effective for treating a disease or condition. A “therapeutically acceptable salt” as used herein is a pharmaceutically acceptable salt of arimoclomol in the composition of the present technology, which, when used in a therapeutically effective amount, is effective for treating a disease, condition, or syndrome.

In a further embodiment, the present disclosure provides a composition comprising a therapeutically effective amount of a compound of Formula I, wherein the compound of Formula I is a compound having a structure of Formula IA:

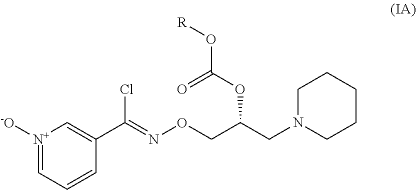

wherein R is selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a still further embodiment, the present disclosure provides a composition comprising a therapeutically effective amount of a compound of Formula IA, wherein the compound of Formula IA is a compound having a structure selected from:

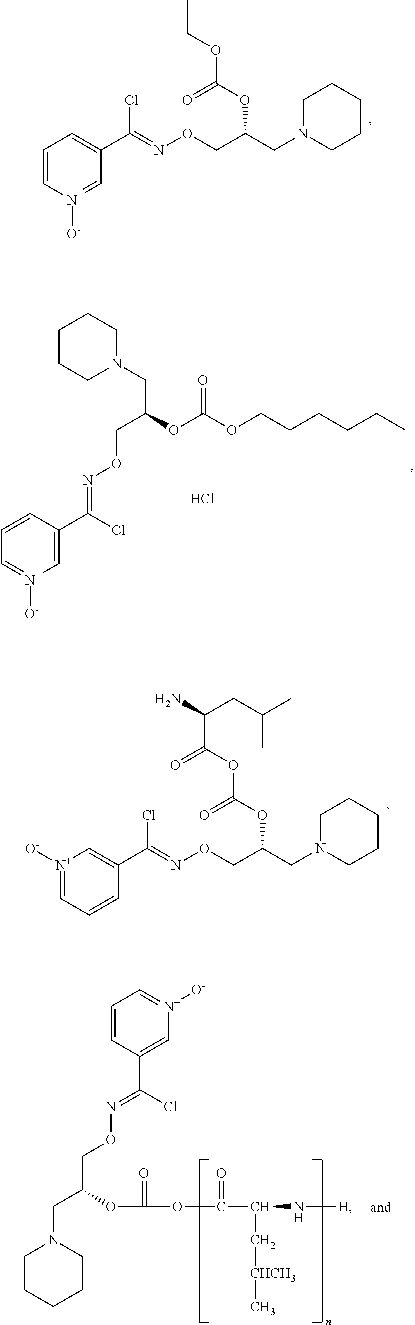

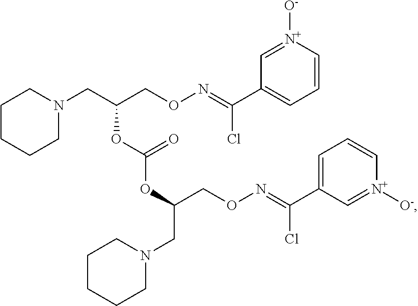

wherein n is 1-50;
or a pharmaceutically acceptable salt thereof.

In a still further embodiment, the present disclosure provides a composition comprising a therapeutically effective amount of a compound of Formula IA, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In a further embodiment, the present disclosure provides a composition comprising a therapeutically effective amount of a compound of Formula I, wherein the compound of Formula I is a compound having a structure of Formula IB:

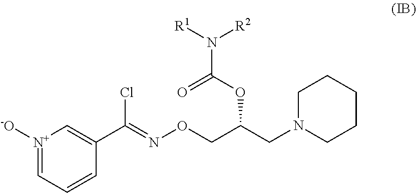

wherein R¹ and R² are independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a further embodiment, the present disclosure provides a composition comprising a therapeutically effective amount of a compound of Formula IB, wherein the compound of Formula IB is a compound having a structure selected from:

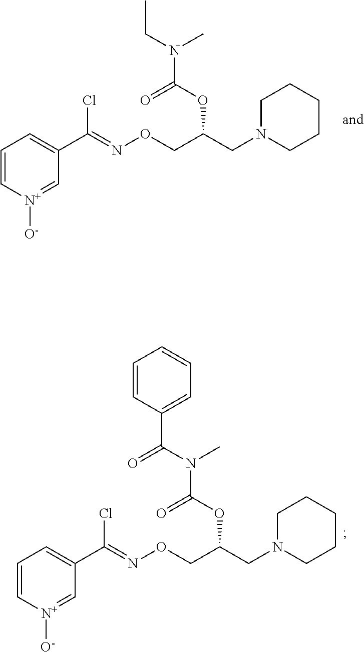 [SMILES: CCN(C)C(=O)O[C@@H](CO/N=C(\Cl)c1ccc[n+]([O-])c1)CN1CCCCC1 ; CN(C(=O)O[C@@H](CO/N=C(\Cl)c1ccc[n+]([O-])c1)CN1CCCCC1)C(=O)c1ccccc1]

or a pharmaceutically acceptable salt thereof.

In a further embodiment, the present disclosure provides a composition comprising a therapeutically effective amount of a compound of Formula I, wherein the compound of Formula I is a compound of Formula IC:

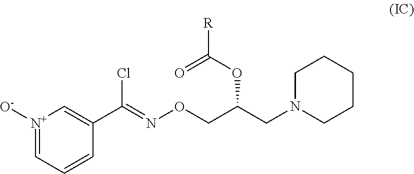

wherein R is selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a further embodiment, the present disclosure provides a composition comprising a therapeutically effective amount of a compound of Formula IC, wherein the compound of Formula IC is a compound having a structure selected from:

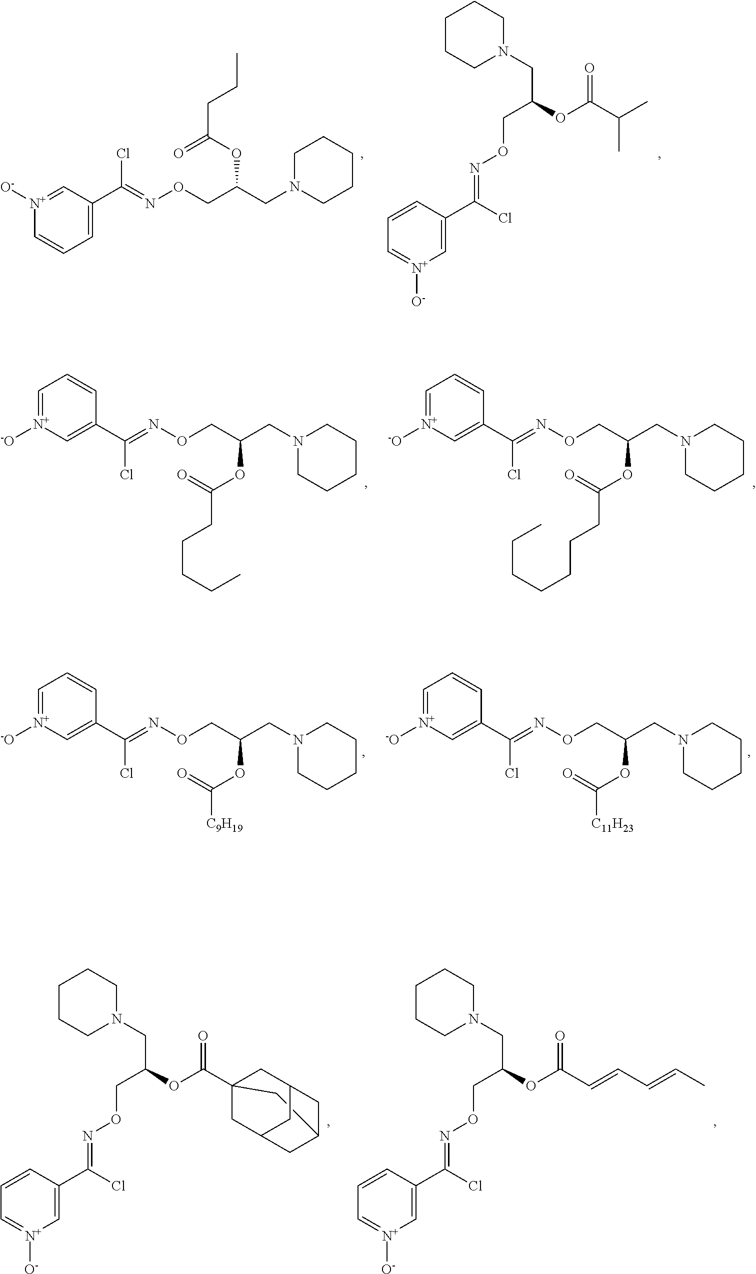

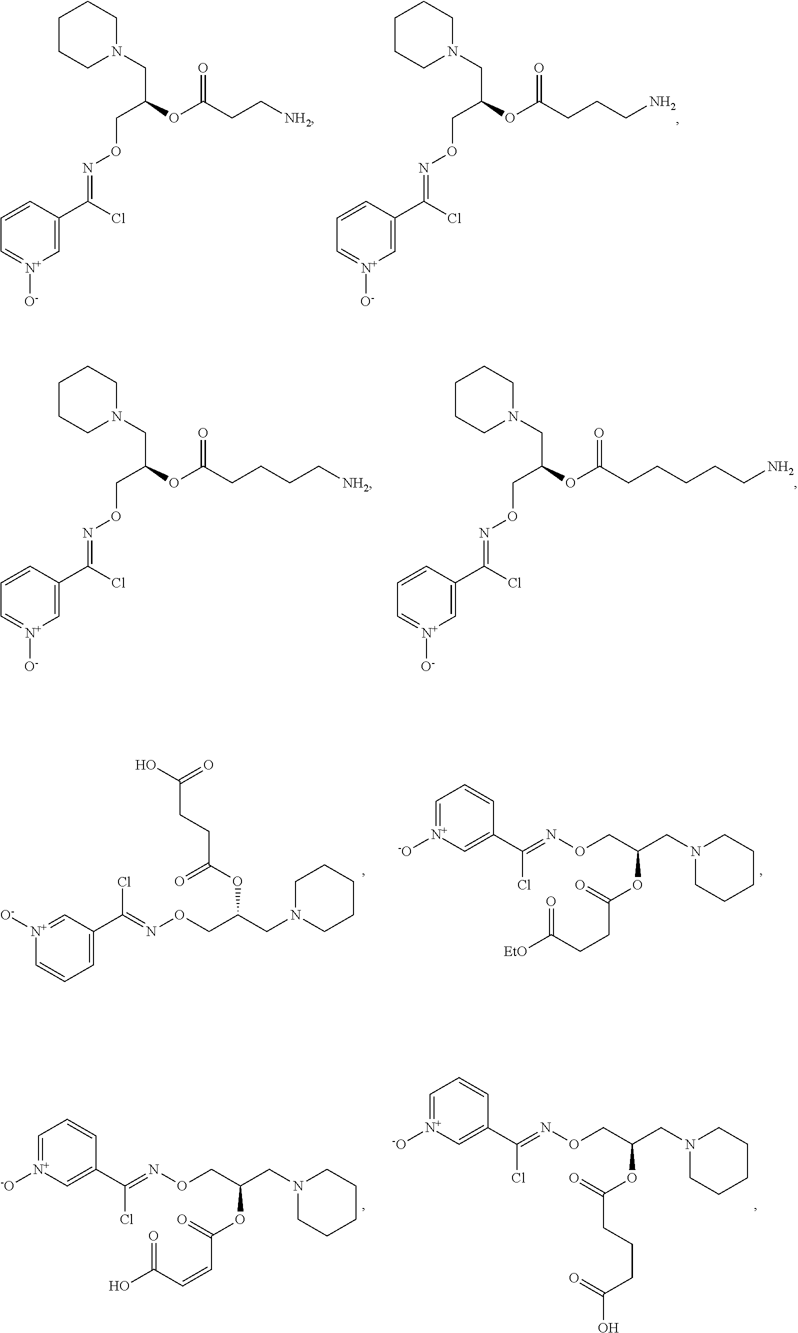

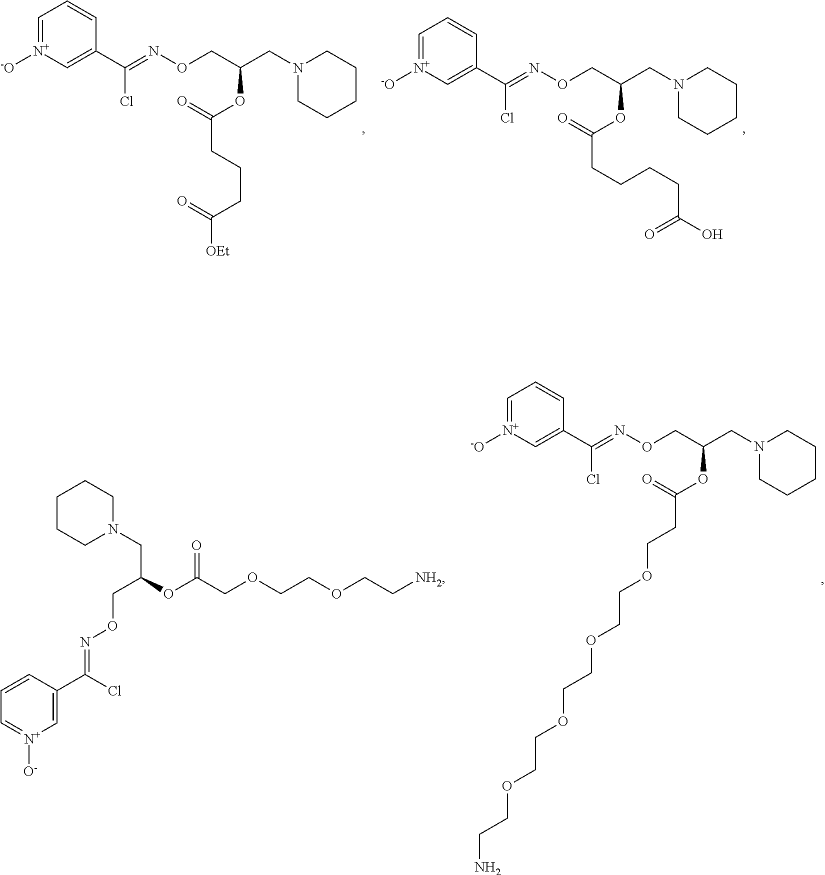

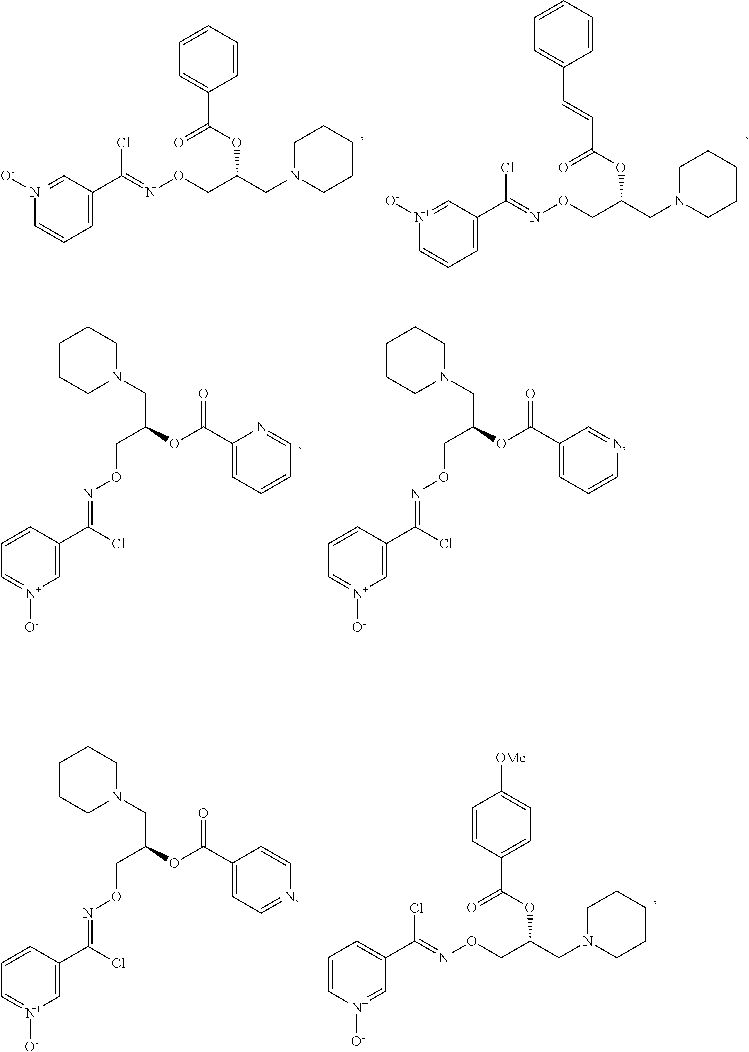

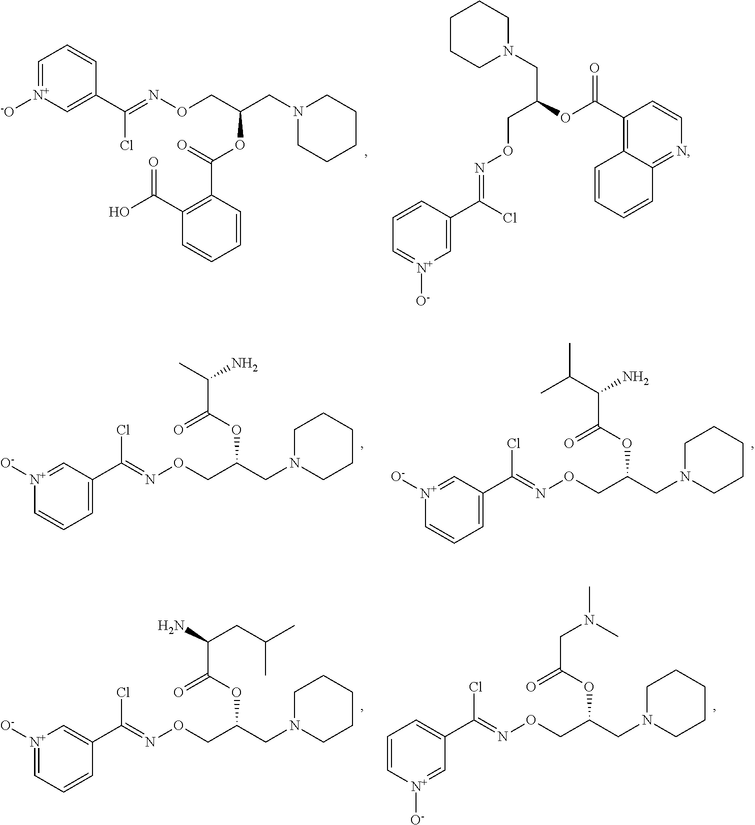

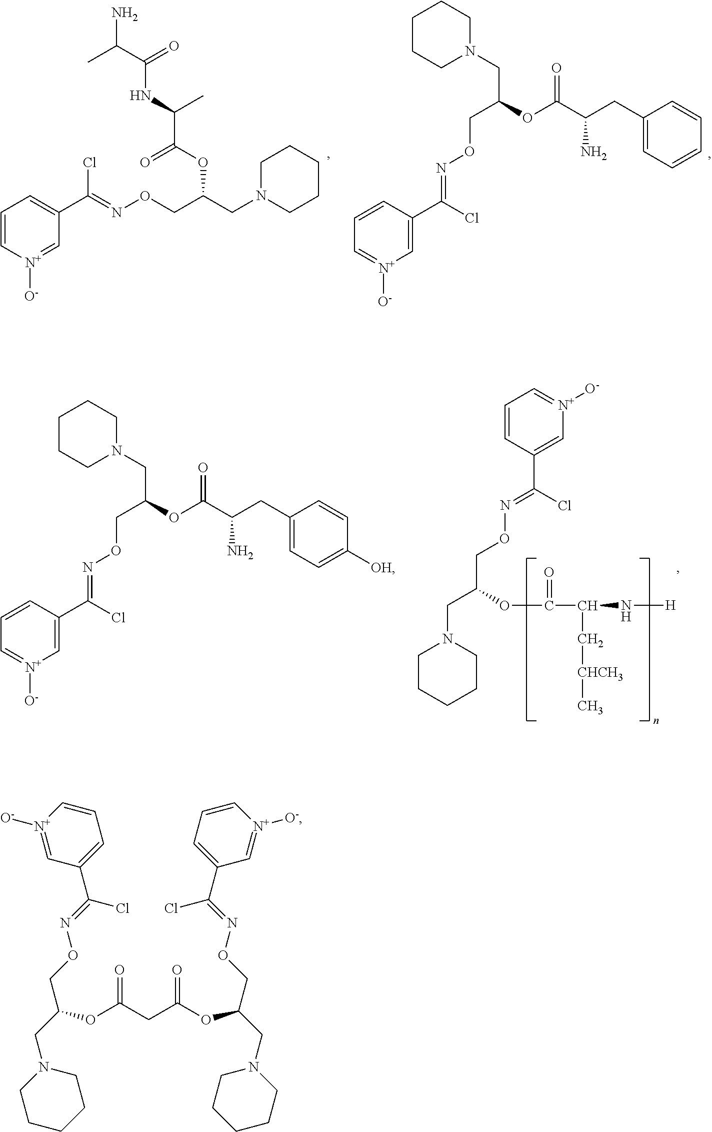

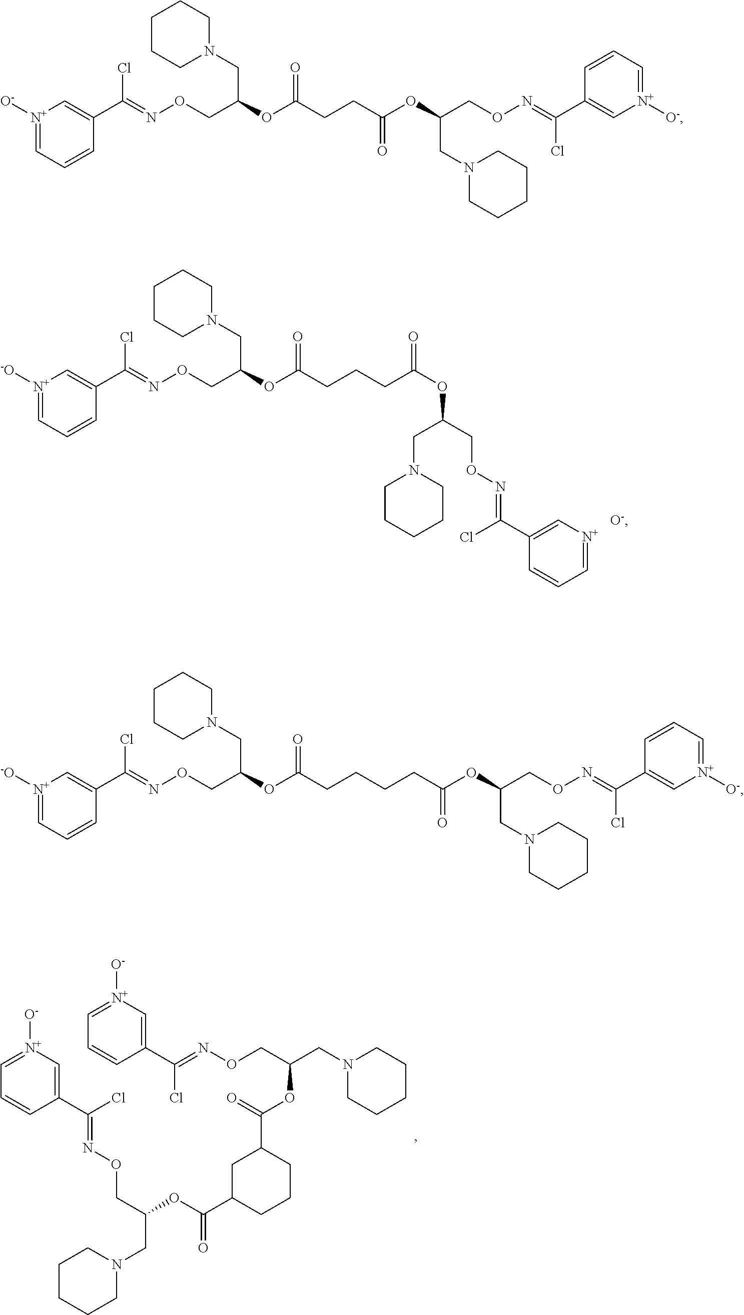

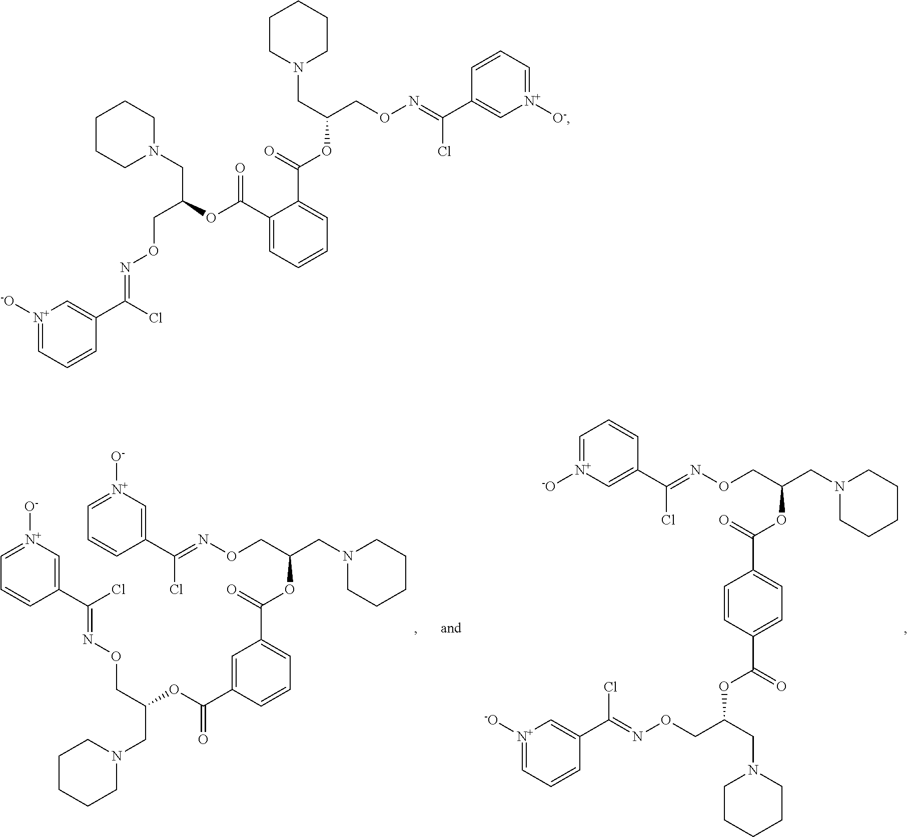

wherein n is 1-50;
or a pharmaceutically acceptable salt thereof.

In a still further embodiment, the present disclosure provides a composition comprising a therapeutically effective amount of a compound of Formula IC, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In a still further embodiment, the present disclosure provides a composition comprising a therapeutically effective amount of a compound of Formula IC, wherein the compound of Formula IC is a compound having a structure selected from:

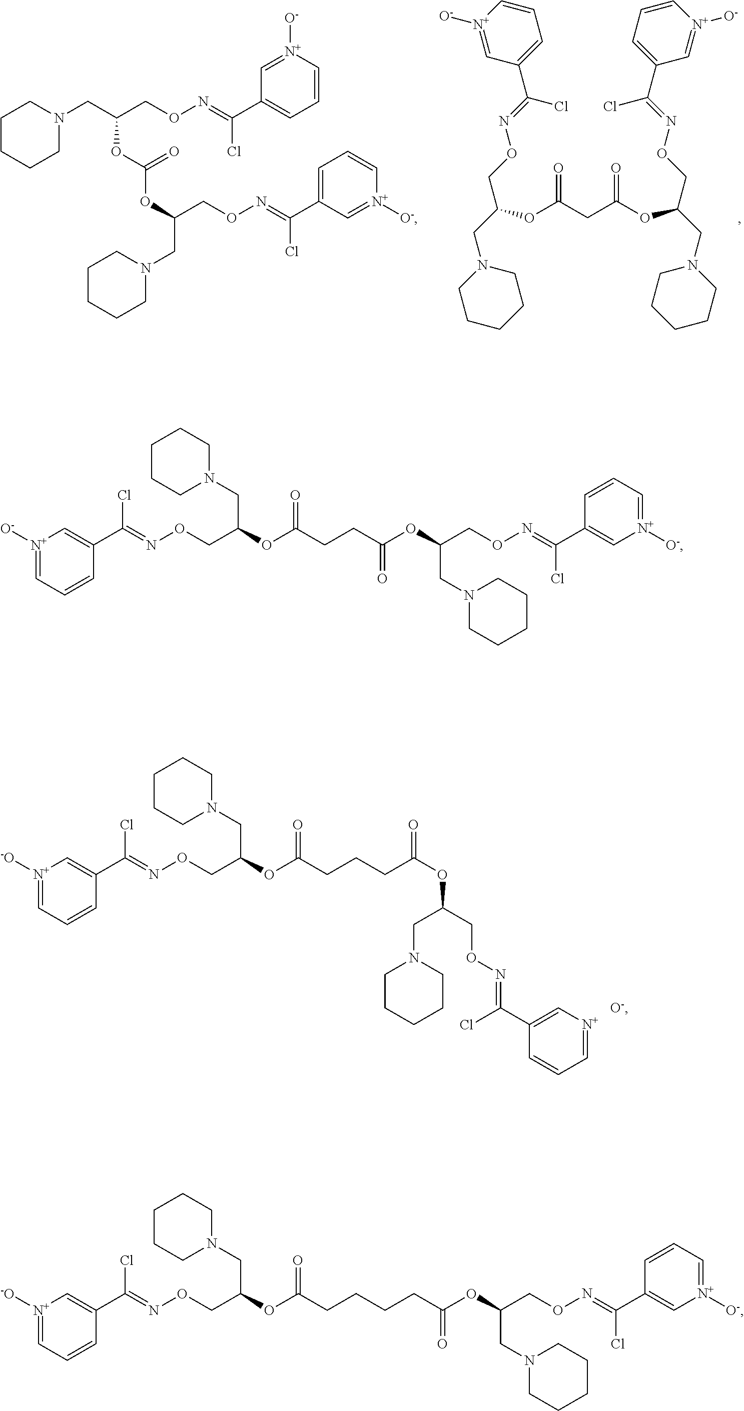

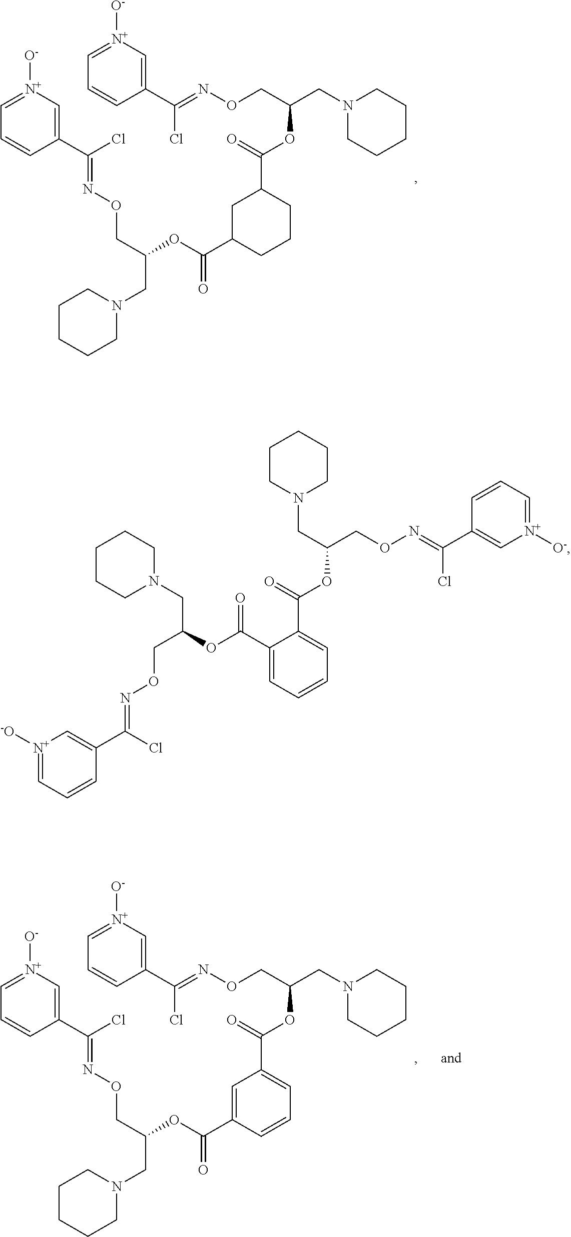

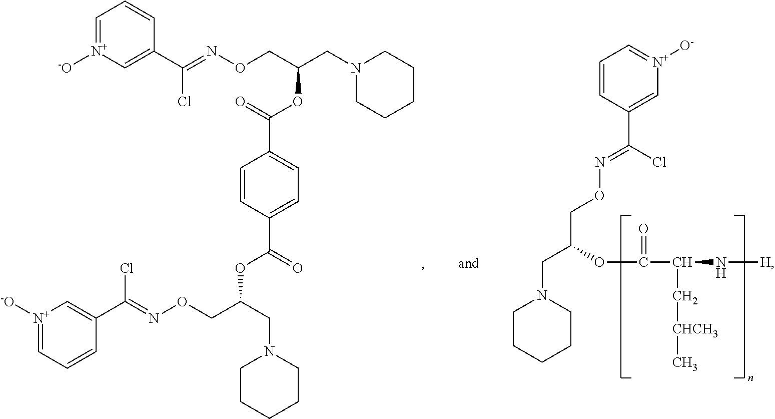

wherein n is 1-50
or a pharmaceutically acceptable salt thereof.

In a further embodiment, the present disclosure provides a composition comprising a therapeutically effective amount of a compound of Formula I, wherein the compound of Formula I is a compound of Formula ID:

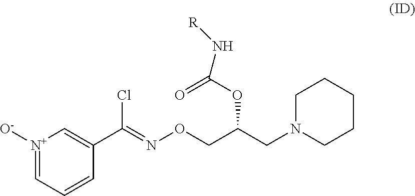

wherein R is selected from the group consisting of alkenyl, alkenyl, aminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a further embodiment, the present disclosure provides a composition comprising a therapeutically effective amount of a compound of Formula ID, wherein the compound of Formula ID is a compound having a structure selected from:

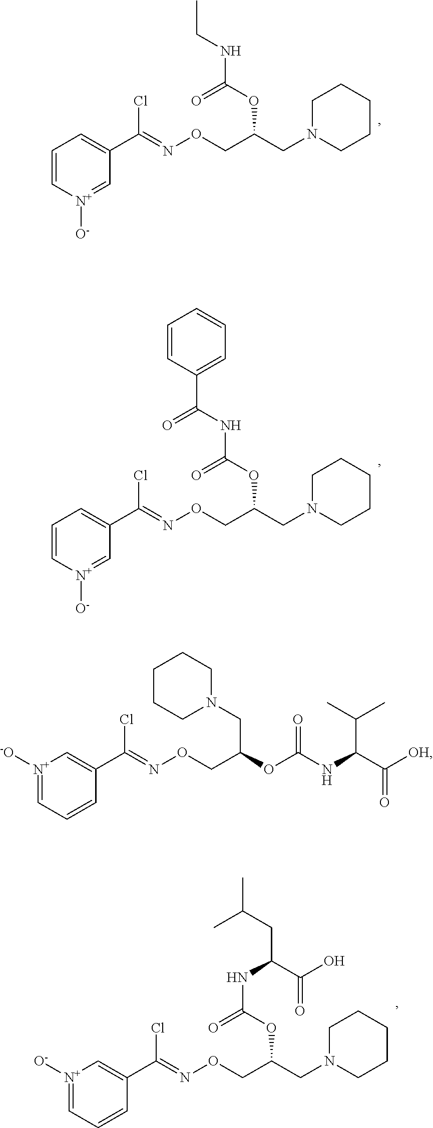

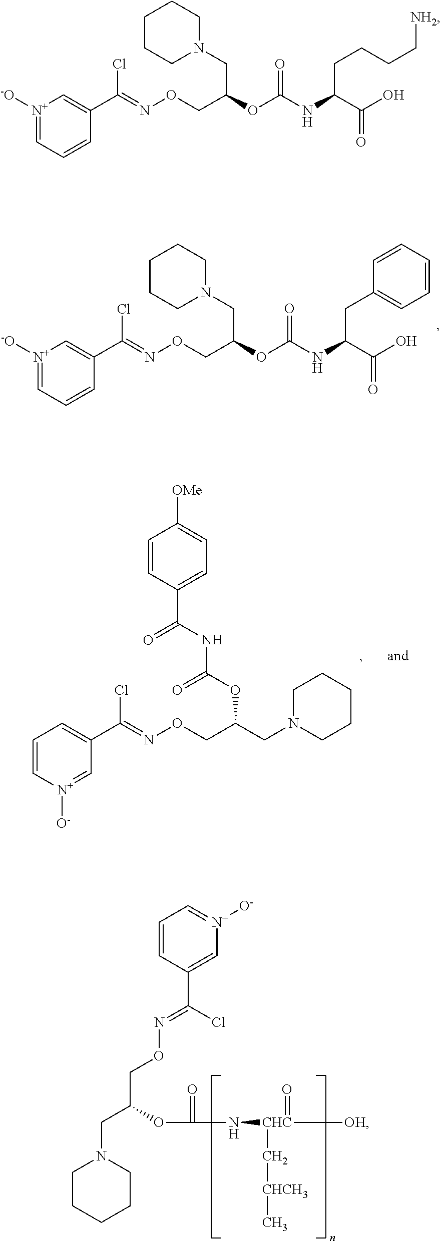

wherein n is 1-50;
or a pharmaceutically acceptable salt thereof.

In a still further embodiment, the present disclosure describes the composition comprising the therapeutically effective amount of the compound of Formula IC, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In an embodiment, the present disclosure describes a composition comprising:
a therapeutically effective amount of a compound of Formula II:

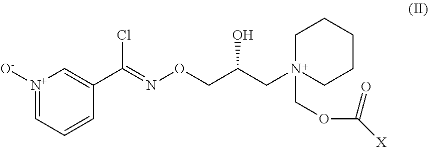

wherein X is selected from the group consisting of R, [O—R], [NR¹R²], and [NRH]
wherein O is oxygen, N is nitrogen, H is hydrogen, and
R, R¹ and R² are independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a still further embodiment, the present disclosure provides a composition comprising a therapeutically effective amount of a compound of Formula II, wherein the compound of Formula II is selected from:

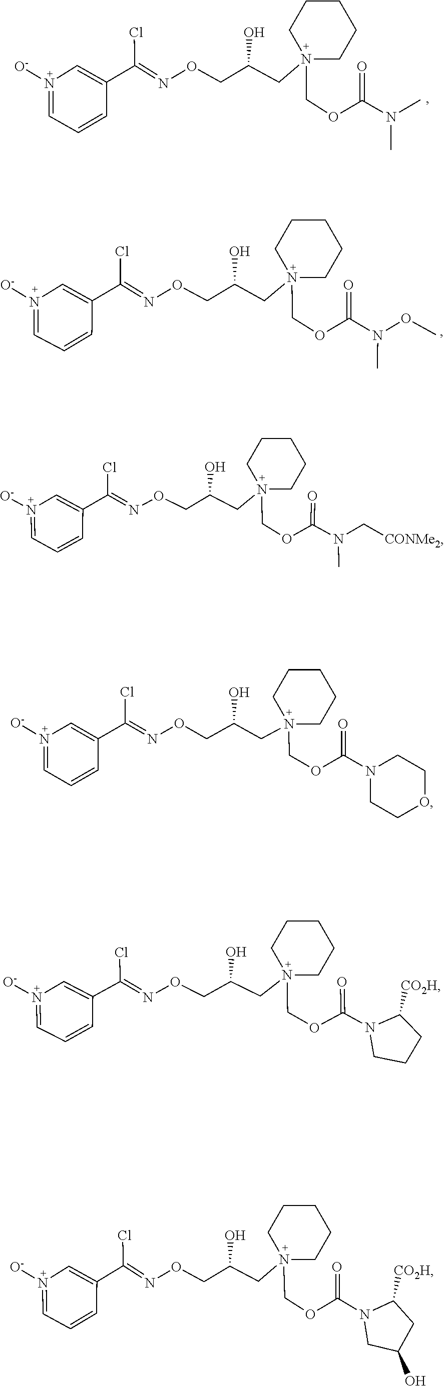

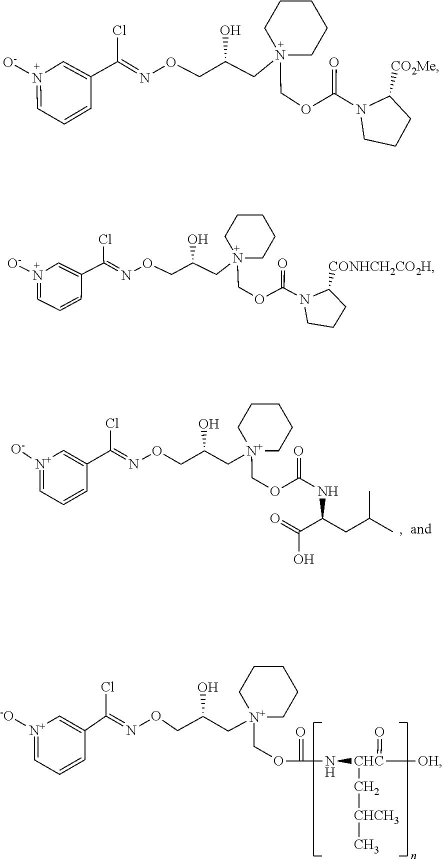

wherein n is 1-50;
or a pharmaceutically acceptable salt thereof.

In a still further embodiment, the present disclosure provides a composition comprising a therapeutically effective amount of a compound of Formula II, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In an embodiment, the present disclosure provides a composition comprising a therapeutically effective amount of a compound of Formula III:

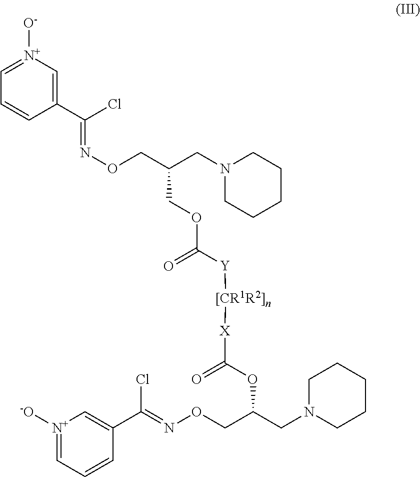

wherein X is selected from the group consisting of O, N, or absent,
Y is selected from the group consisting of O, N, or absent,
n is 1-10,
R¹ and R² are selected independently from each other and for each CR¹R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a further embodiment, the present disclosure provides a composition comprising a therapeutically effective amount of a compound of Formula III, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In an embodiment, the present disclosure provides a composition comprising a therapeutically effective amount of a compound of Formula IV:

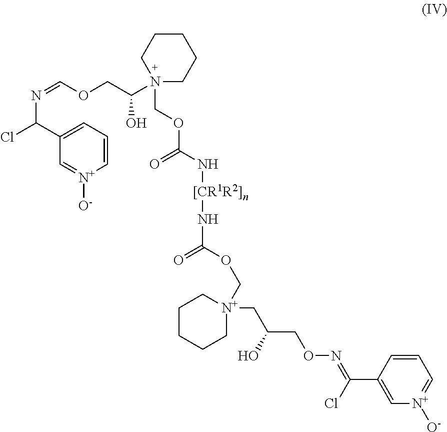

n is 1-10,
R¹ and R² are selected independently from each other and for each CR₁R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a further embodiment, the present disclosure provides a composition comprising a therapeutically effective amount of a compound of Formula IV, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In an embodiment, the present disclosure provides a composition comprising a therapeutically effective amount of a compound of Formula V

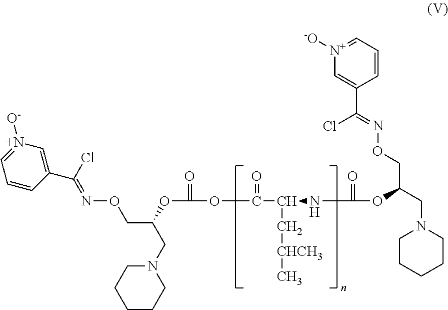

wherein n is 1-50,
or a pharmaceutically acceptable salt thereof.

In a further embodiment, the present disclosure provides a composition comprising a therapeutically effective amount of a compound of Formula V, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In an embodiment, the present disclosure provides a composition comprising a therapeutically effective amount of a compound of Formula VI:

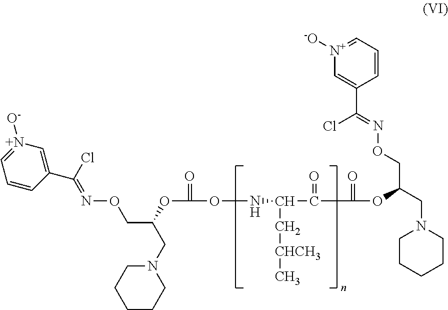

wherein n is 1-50;
or a pharmaceutically acceptable salt thereof.

In a further embodiment, the present disclosure provides a composition comprising a therapeutically effective amount of a compound of Formula VI, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In an embodiment, the present disclosure provides a composition comprising a therapeutically effective amount of a compound of Formula VII:

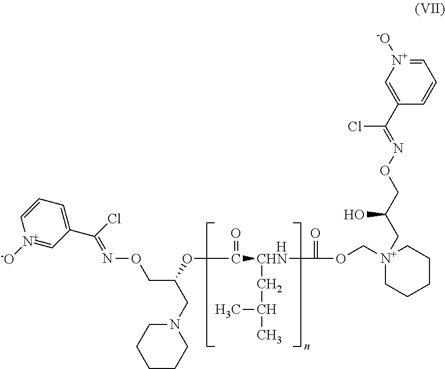

wherein n is 1-50,
or a pharmaceutically acceptable salt thereof.

In certain embodiment, the present disclosure provides a composition comprising a therapeutically effective amount of a compound of Formula I to Formula VII, wherein the pharmaceutically acceptable salt is selected from sulfate, citrate, acetate, oxalate, chloride, hydrochloride, bromide, hydrobromide, iodide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate. In a still further embodiment, the pharmaceutically acceptable salt is citrate.

In an embodiment, the present disclosure provides a composition comprising a therapeutically effective amount of a compound having a structure selected from

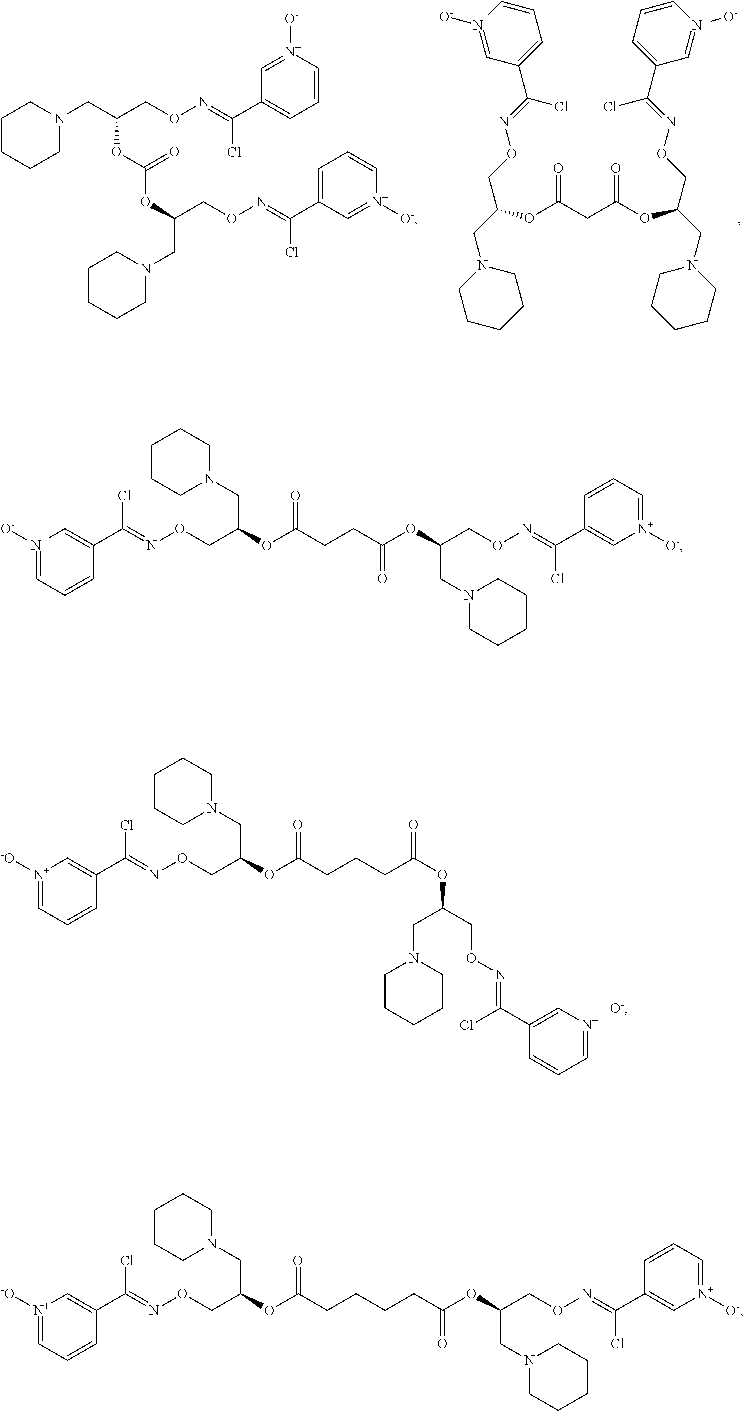

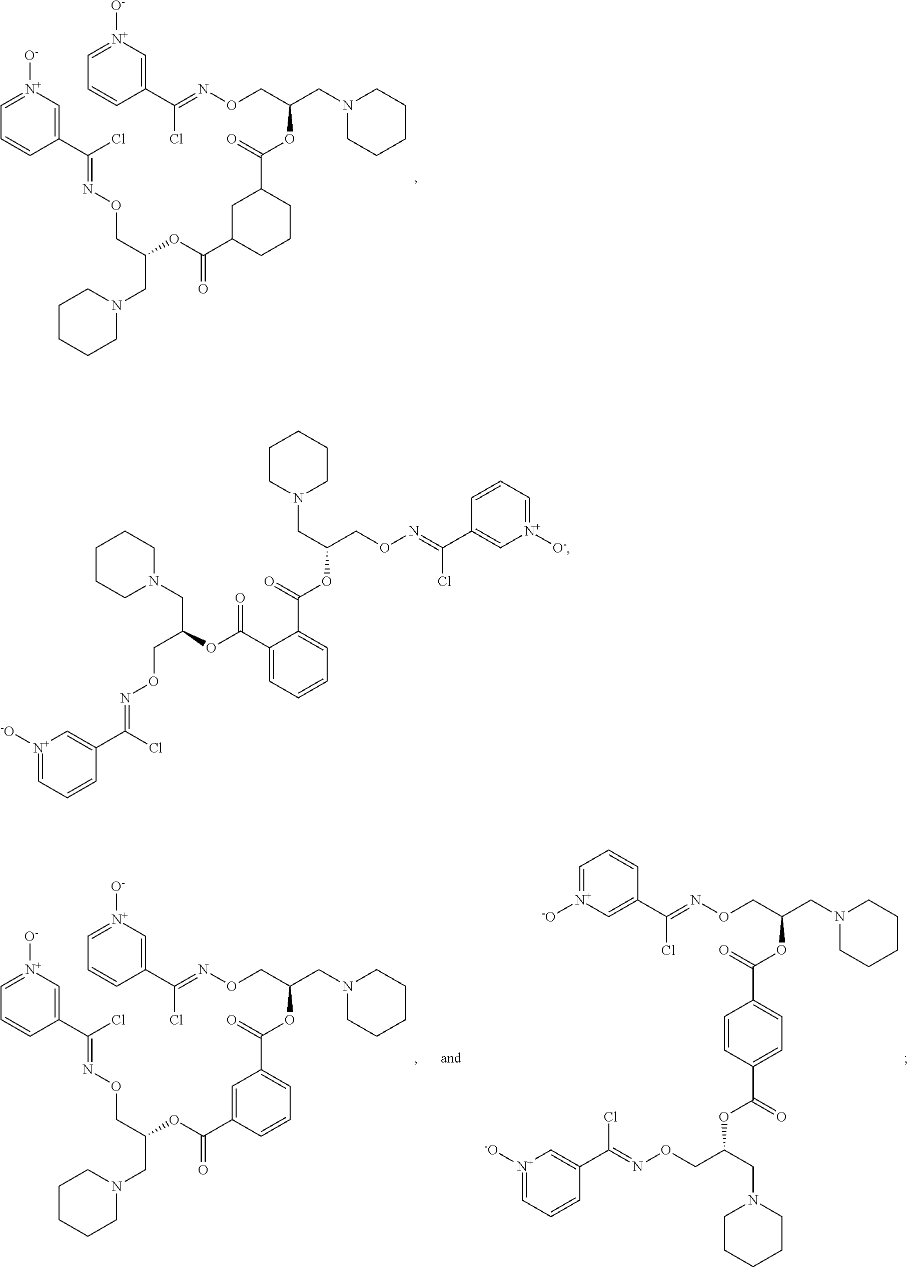

or a pharmaceutically acceptable salt thereof.

In an embodiment, the present disclosure provides a composition comprising a therapeutically effective amount of a compound having a structure selected from

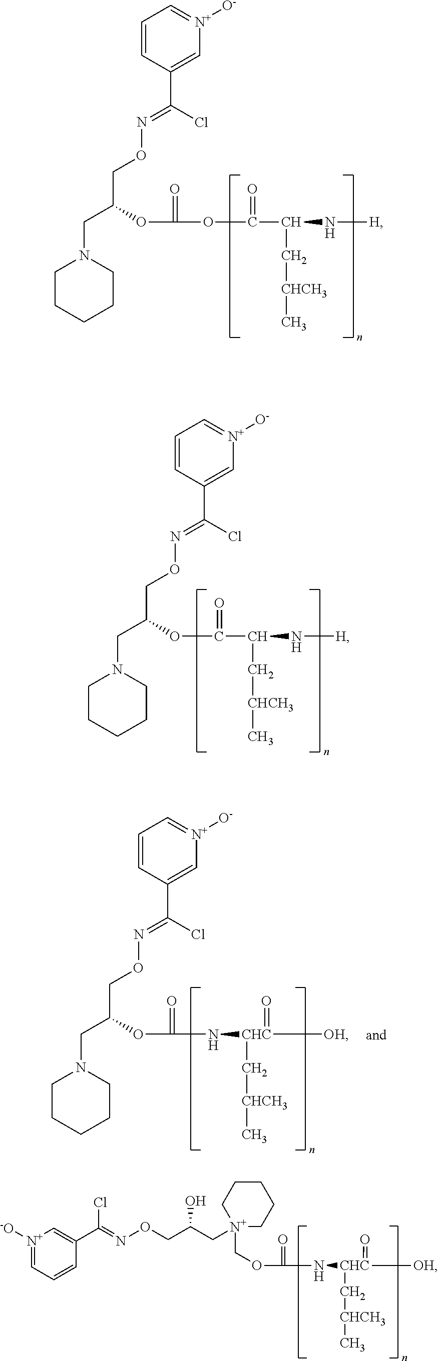

wherein n is 1-50; or a pharmaceutically acceptable salt thereof. Alternatively, n is 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In a further embodiment, the present disclosure provides a composition comprising a therapeutically effective amount of a compound of Formula I to Formula VII, wherein the compound of Formula I to Formula VII is present as a stereoisomer of said compound. In a still further embodiment, the compound of Formula I or Formula II is present as a stereoisomer and comprises a sub-structure selected from (+)-(R)—N-[2-hydroxy-3-(1-piperidinyl)-propoxy]-pyridine-1-oxide-3-carboximidoyl chloride, and (−)-(S)—N-[2-hydroxy-3-(1-piperidinyl)-propoxy]-pyridine-1-oxide-3-carboximidoyl chloride, (Z)—(R)—N-[2-hydroxy-3-(1-piperidinyl)-propoxy]-pyridine-1-oxide-3-carboximidoyl chloride, (E)-(R)—N-[2-hydroxy-3-(1 piperidinyl)-propoxy]-pyridine-1-oxide-3-carboximidoyl chloride, (Z)—(S)—N-[2-hydroxy-3-(1-piperidinyl)-propoxy]-pyridine-1-oxide-3-carboximidoyl chloride; and (E)-(S)—N-[2-hydroxy-3-(1-piperidinyl)-propoxy]-pyridine-1-oxide-3-carboximidoyl chloride.

In a still further embodiment, the present disclosure provides a composition comprising a therapeutically effective amount of a compound of Formula I to Formula VII, wherein the at least one excipient is selected from antiadherents, binders, coatings, disintegrants, gel forming agents, fillers, flavors and colors, glidants, lubricants, preservatives, sorbents and sweeteners.

“Excipients” as it relates to the present technology means pharmaceutically inert compounds which may include one or more of the following types: antiadherents, binders, coatings, disintegrants, gel forming agents, fillers, flavors and colors, glidants, lubricants, preservatives, sorbents and sweeteners.

In a further embodiment, the present disclosure provides a composition comprising a therapeutically effective amount of a compound of Formula I to Formula VII, wherein the at least one additional active pharmaceutical ingredient is selected from bimoclomol, miglustat, eliglustat, N-acetyl-L-leucine and 2-hydroxypropyl-β-cyclodextrin, and combinations thereof. In a still further embodiment, the at least one additional active pharmaceutical ingredient is selected from miglustat, N-acetyl-L-leucine, and combinations thereof. In a still further embodiment, the at least one additional active pharmaceutical ingredient is a combination of miglustat and N-acetyl-L-leucine.

In a further embodiment, the present disclosure provides a composition comprising a therapeutically effective amount of a compound of Formula I to Formula VII, the pharmaceutical composition is formulated for parenteral administration.

“Parenteral administration” as it relates to the present technology, means administration by injection, infusion, intravenous such as through a drip line. Parenteral administration may also mean through dermal absorptions such as from a skin patch.

In a further embodiment, the present disclosure provides a composition comprising a therapeutically effective amount of a compound of Formula I to Formula VII, wherein the composition is formulated for oral administration. In a still further embodiment, the composition is a solid oral dosage formulation.

“Solid oral dosage formulation” as it relates to the present technology means dosage forms that include but are not limited to sublingual, gummy, chewable tablet, rapidly dissolving tablet, tablet, capsule, caplet, troche, lozenge, powder, oral thin film (OTF), oral strip, rectal film, or suppository. In some embodiments, the dosage forms are to be administered orally. Preferred oral administration forms are capsule, tablet, solutions and OTF. Solid oral dosage formulations can optionally include one or more of the following types of excipients: antiadherents, binders, coatings, disintegrants, gel forming agents, fillers, flavors and colors, glidants, lubricants, preservatives, sorbents and sweeteners.

Other compounds which may be included by admixture are, for example, medically inert ingredients, e.g., solid and liquid diluents, such as lactose, dextrose, saccharose, cellulose, starch or calcium phosphate for tablets or capsules, olive oil or ethyl oleate for soft capsules and water or vegetable oil for suspensions or emulsions; lubricating agents such as silica, talc, stearic acid, magnesium or calcium stearate, hydrogenated oils, sodium stearyl fumarate, and/or polyethylene glycols; gelling agents such as colloidal clays, polyethylene oxide, hydroxypropyl methyl cellulose, or carbomers; thickening agents such as gum tragacanth or sodium alginate, binding agents such as starches, arabic gums, gelatin, methylcellulose, carboxymethylcellulose or polyvinylpyrrolidone (povidone); disintegrating agents such as starch, alginic acid, alginates, crospovidone, or sodium starch glycolate; effervescing mixtures; dyestuff; sweeteners; wetting agents such as lecithin, polysorbates, poloxamer, sorbitan monoesters, glyceryl monooleates, or laurylsulfates; and other therapeutically acceptable accessory ingredients, such as humectants, preservatives, buffers and antioxidants, which are known additives for such formulations. In a still further embodiment, the solid oral dosage formulation is selected from a sublingual, a gummy, a chewable tablet, a rapidly dissolving tablet, a tablet, a capsule, a caplet, a troche, a lozenge, an oral powder, a solution, a thin strip, an oral thin film (OTF), an oral strip, and a rectal film.

In a still further embodiment, the present disclosure provides a composition comprising a therapeutically effective amount of a compound of Formula I to Formula VII, wherein the composition is a liquid oral dosage formulation. “Liquid oral dosage form” as it relates to the present technology means solutions, syrups, emulsions, or suspensions. The syrups may contain as carrier, for example, saccharose or saccharose with glycerol and/or mannitol and/or sorbitol and/or dextrin. In particular a syrup for diabetic subjects can contain as carriers only products, for example sorbitol, which does not metabolize to glucose, or which metabolizes to only a very small amount of glucose. The suspensions and the emulsions may contain a carrier, for example a natural gum, agar, sodium alginate, pectin, methylcellulose, carboxymethylcellulose or polyvinyl alcohol. Liquid oral formulations of the present technology can also be included in a solution, a suspension or a slurry in an aqueous liquid or a non-aqueous liquid. The formulation can be an emulsion, such as an oil-in-water liquid emulsion or a water-in-oil liquid emulsion. The oils can be administered by adding the purified and sterilized liquids to a prepared enteral formula, which is then placed in the feeding tube of a subject who is unable to swallow. In a still further embodiment the liquid oral dosage formulation is selected from syrups, emulsions, solutions, and suspensions.

In a still further embodiment, the present disclosure provides a composition comprising a therapeutically effective amount of a compound of Formula I to Formula VII, wherein the therapeutically effective amount of the compound of Formula I ranges from about 1 to about 5000 mg per day. In a still further embodiment, the therapeutically effective amount ranges from about 50 mg to about 2500 mg, alternatively about 100 mg to about 2000 mg, alternatively about 200 mg to about 1500 mg, alternatively about 500 mg to about 1000 mg per day.

In a still further embodiment, the present disclosure provides a composition comprising a therapeutically effective amount of a compound of Formula I to Formula VII, wherein the therapeutically effective amount of the compound of Formula I is administered as a unit dose. “Unit dose form” as it related to the present technology means a single entity of a solid therapeutic dosage form (e.g., 1 capsule, 1 tablet) or a single volume dispensed from a non-solid dosage form (e.g., 5 mL of a liquid or syrup). Such a unit dose form can be from about 0.1 mg to about 400 mg per day, alternatively from about 0.1 mg to about 300 mg per day, about 0.1 mg to about 200 mg per day, alternatively about 0.1 mg to about 100 mg per day.

Alternatively, the solid or liquid oral dosage may be opened and the contents sprinkled on 15-20 mL soft food such as applesauce and/or pudding. Alternatively, the solid oral dosage may be opened and the contents dissolved in 15-20 mL of water.

In an embodiment, the present disclosure provides an extended-release composition comprising:
a therapeutically effective amount of a compound of Formula I:

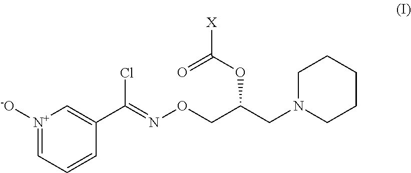

wherein X is selected from the group consisting of R, [O—R], [NR¹R²], and [NRH], where
O is oxygen, N is nitrogen, and H is hydrogen;
R, R¹ and R² are independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof; and
a release controlling agent.

“Extended-release” as it relates to the present disclosure means either a compound (arimoclomol prodrug) of the present invention designed to release the active pharmaceutical ingredient (API) at a delayed or reduced rate compared to free-base arimoclomol, or pharmaceutically acceptable salt thereof, and/or may additionally comprise one or more modifications to the pharmaceutical composition designed to release the active pharmaceutical ingredient (API) at a delayed or reduced rate compared to an unmodified pharmaceutical composition. This delayed or reduced rate of release may result in slower absorption and/or a longer elimination half-life of the API and/or its active metabolites. The delayed or reduced rate of release may be engendered through drug product formulation and/or through the prodrug metabolism.

In a still further embodiment, the present disclosure provides an extended-release composition comprising a therapeutically effective amount of a compound of Formula I, wherein the compound is a compound having a structure of Formula IA:

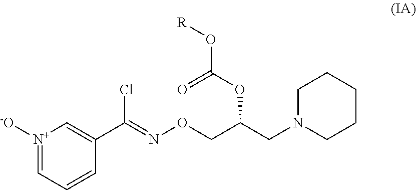

wherein R is selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a still further embodiment, the present disclosure provides an extended-release composition comprising a therapeutically effective amount of a compound of Formula IA, wherein the compound of Formula IA is a compound having a structure selected from:

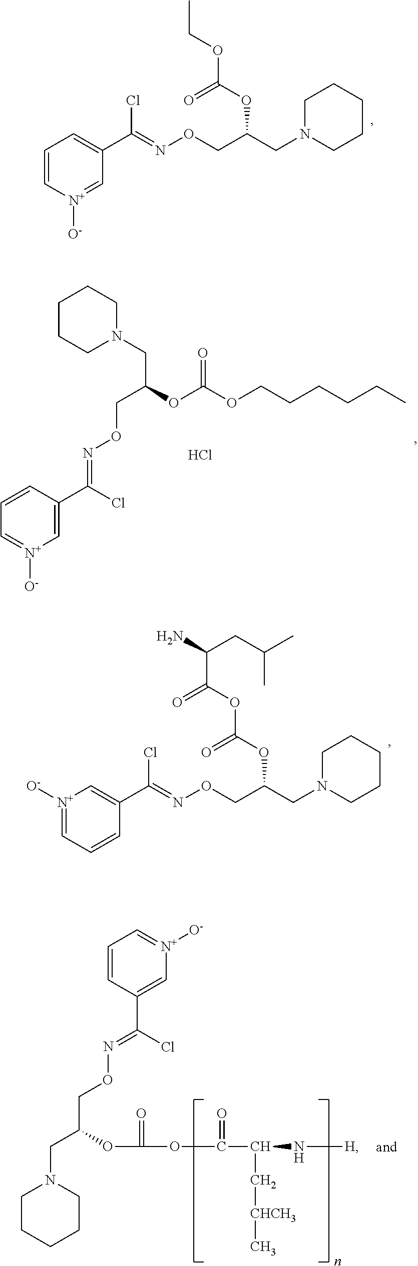

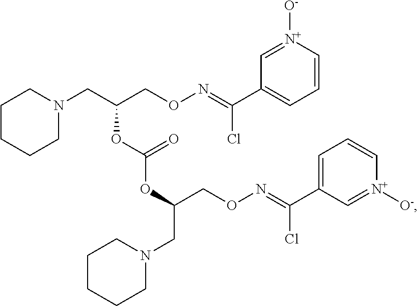

wherein n is 1-50;
or a pharmaceutically acceptable salt thereof.

In a still further embodiment, the present disclosure provides an extended-release composition comprising a therapeutically effective amount of a compound of Formula IA, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In a still further embodiment, the present disclosure provides an extended-release composition comprising a therapeutically effective amount of a compound of Formula I, wherein the compound of Formula I is a compound having a structure of Formula IB:

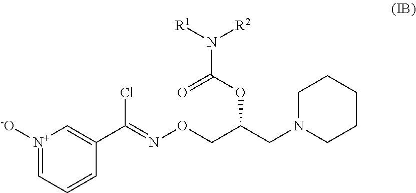

wherein R¹ and R² are independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a still further embodiment, the present disclosure provides an extended-release composition comprising a therapeutically effective amount of a compound of Formula IB, wherein the compound of Formula IB is a compound having a structure selected from:

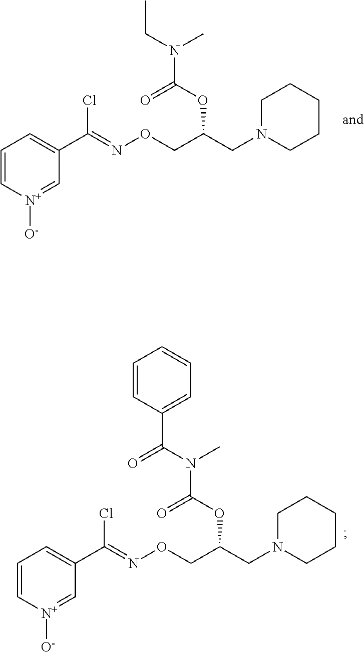 [SMILES: CCN(C)C(=O)O[C@@H](CO/N=C(\Cl)c1ccc[n+]([O-])c1)CN1CCCCC1 ; CN(C(=O)O[C@@H](CO/N=C(\Cl)c1ccc[n+]([O-])c1)CN1CCCCC1)C(=O)c1ccccc1]

or a pharmaceutically acceptable salt thereof.

In a still further embodiment, the present disclosure provides an extended-release composition comprising a therapeutically effective amount of a compound of Formula I, wherein the compound of Formula I is a compound of Formula IC:

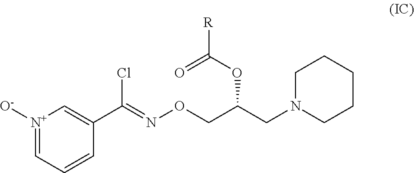

wherein R is selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a still further embodiment, the present disclosure provides an extended-release composition comprising a therapeutically effective amount of a compound of Formula IC, wherein the compound of Formula IC is a compound having a structure selected from:

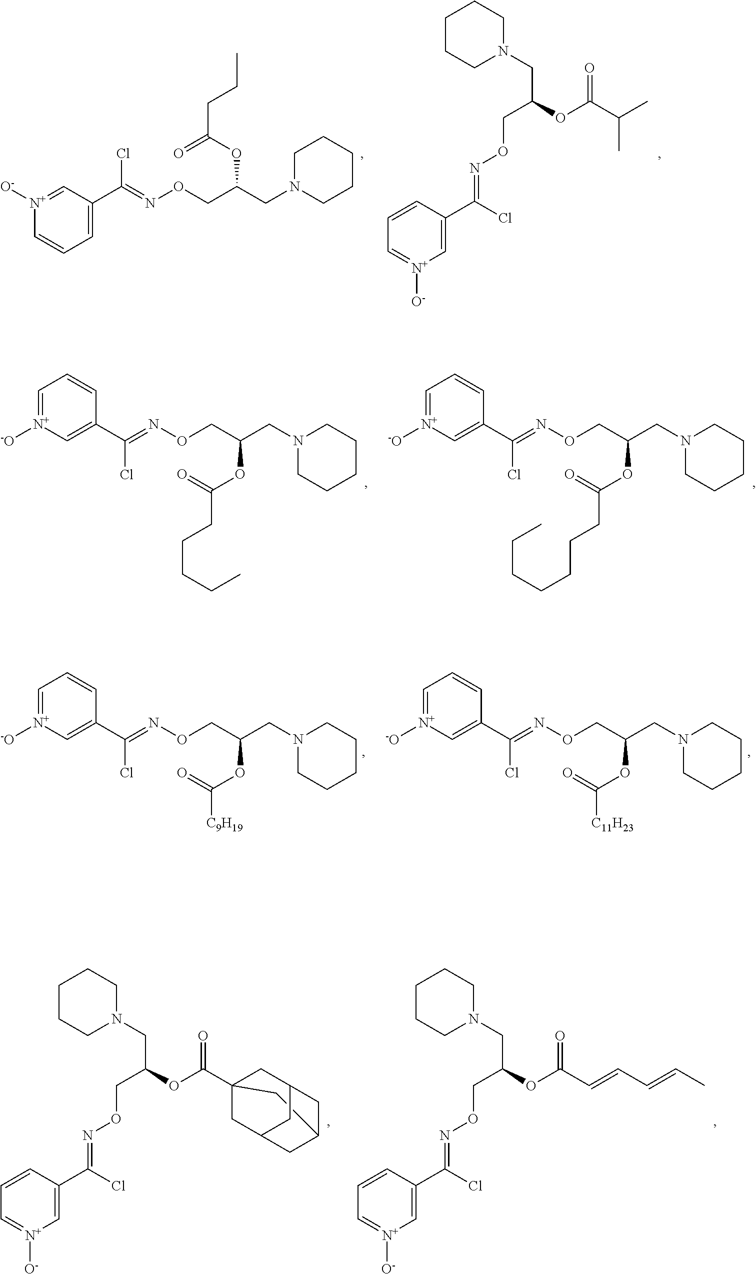

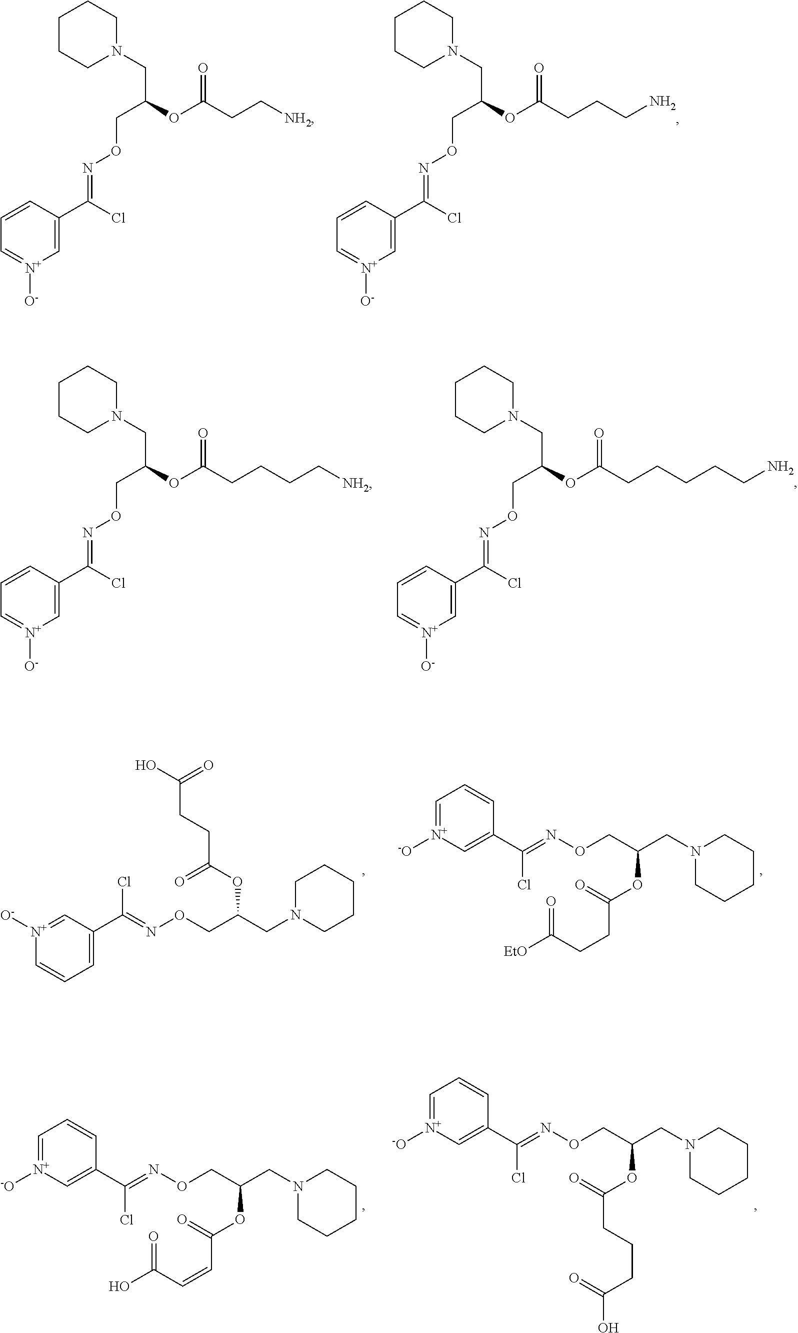

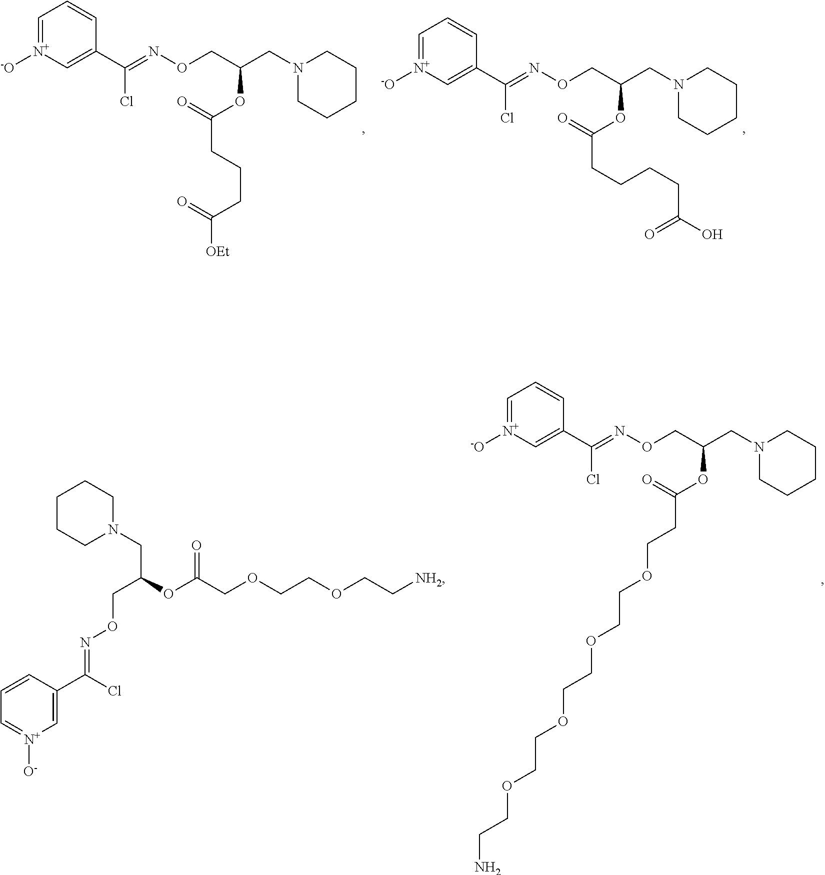

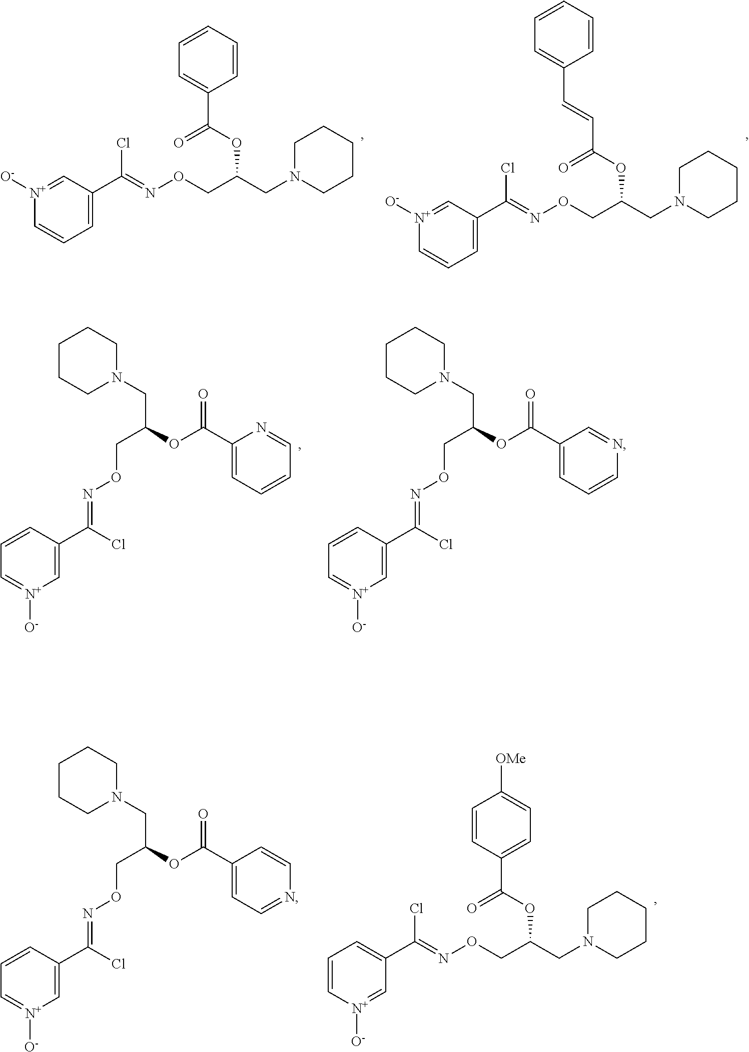

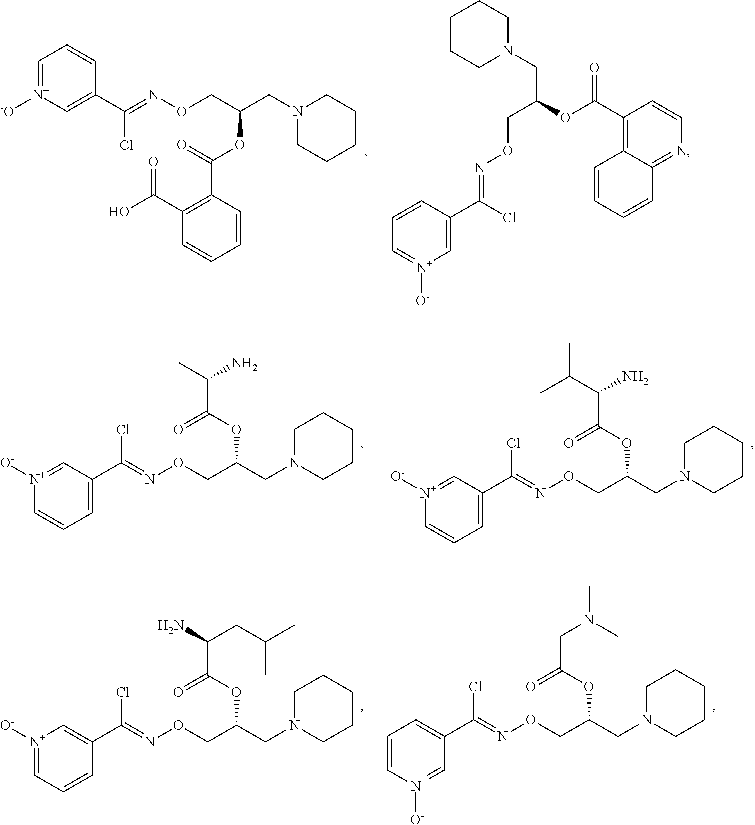

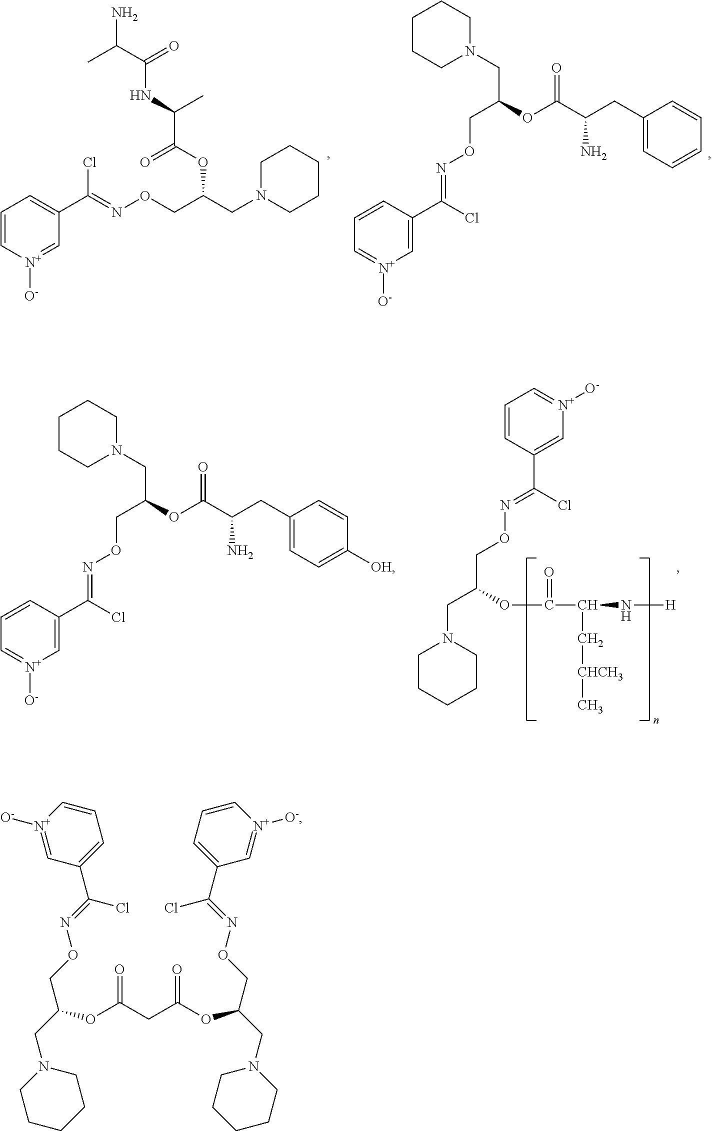

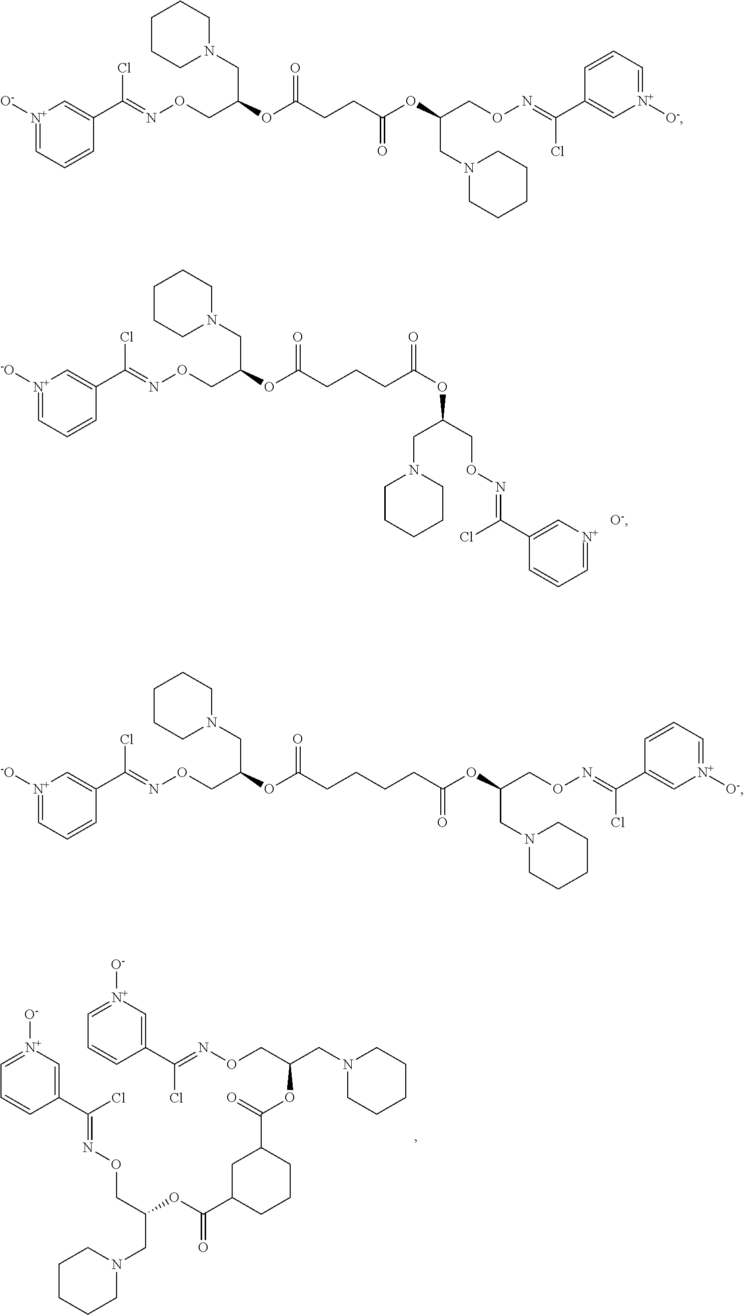

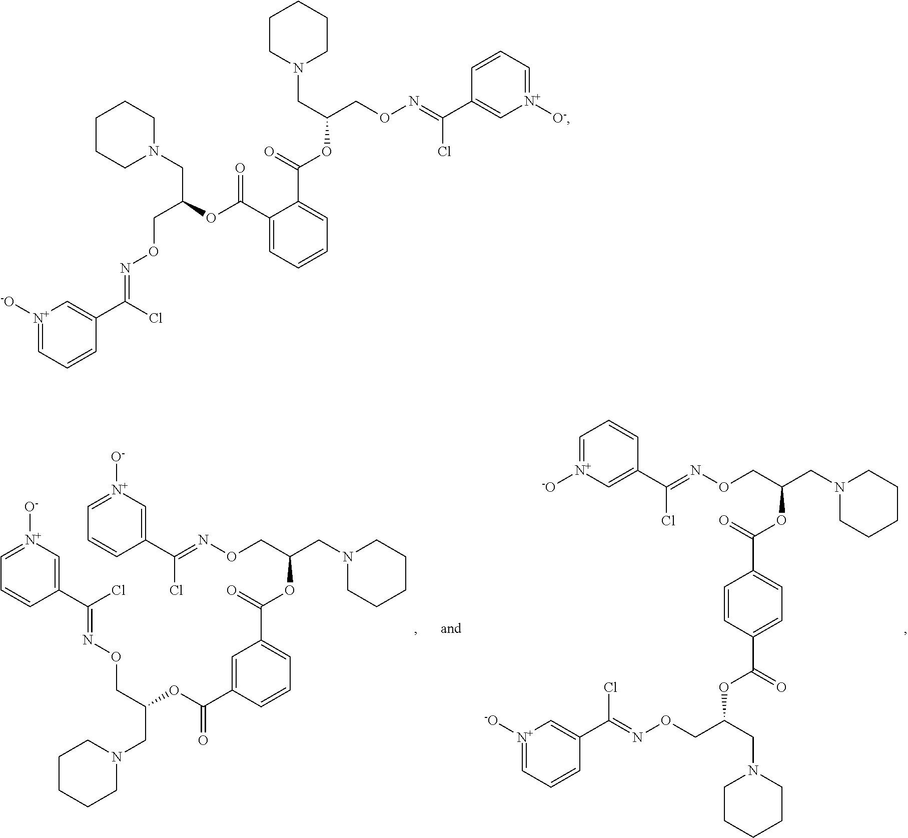

wherein n is 1-50;
or a pharmaceutically acceptable salt thereof.

In a still further embodiment, the present disclosure describes the extended-release composition comprising the therapeutically effective amount of the compound of Formula IC, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In a still further embodiment, the present disclosure describes the extended-release composition comprising the therapeutically effective amount of the compound of Formula IC, wherein the compound of Formula IC is a compound having a structure selected from:

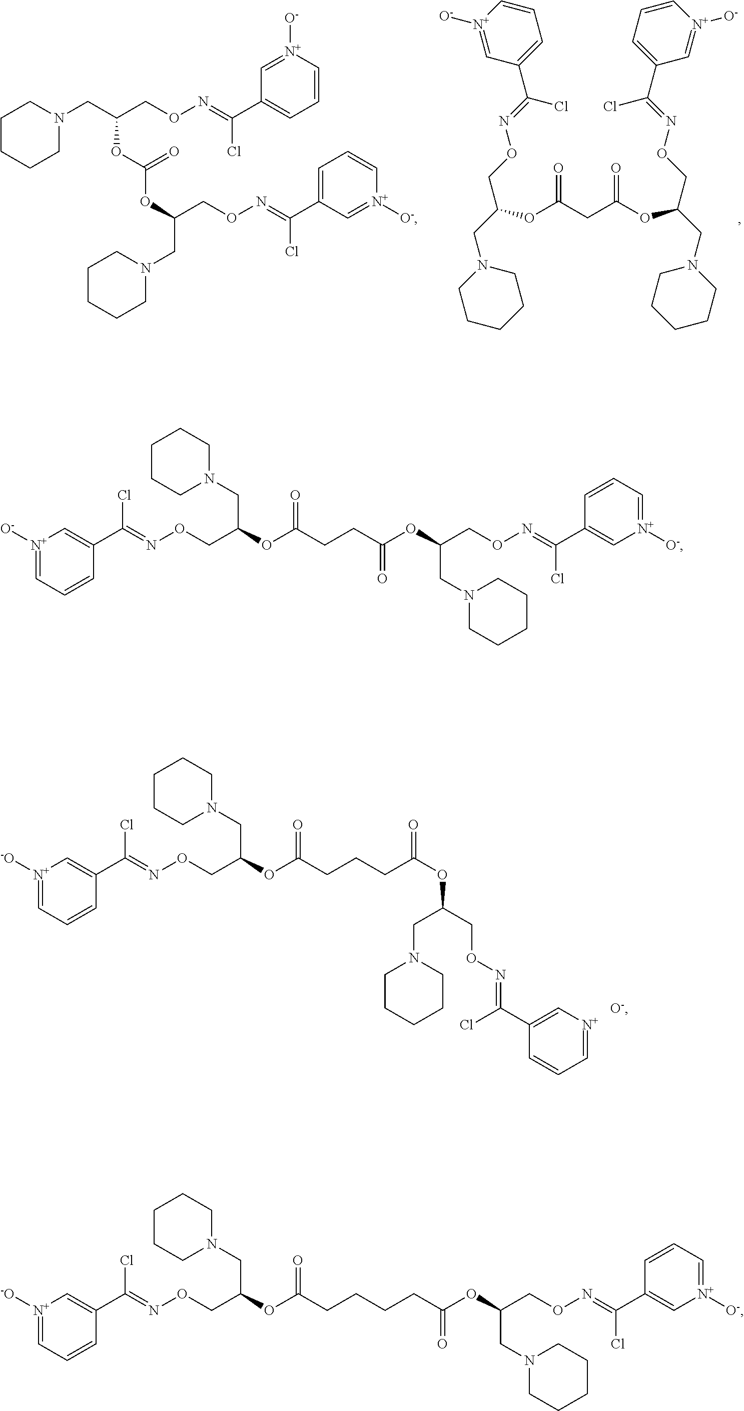

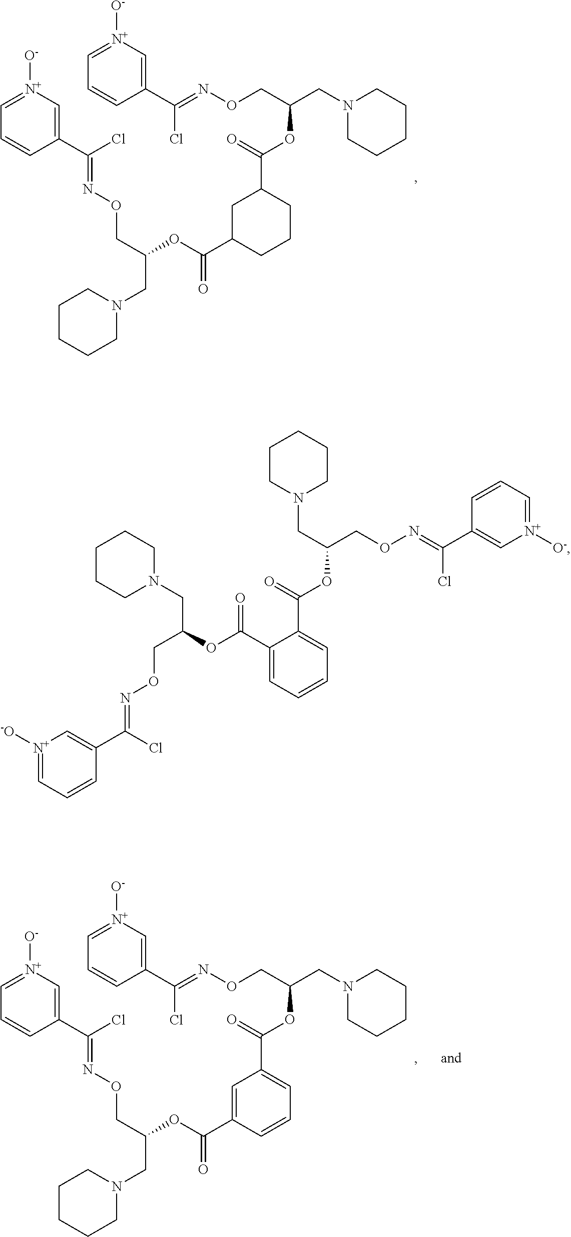

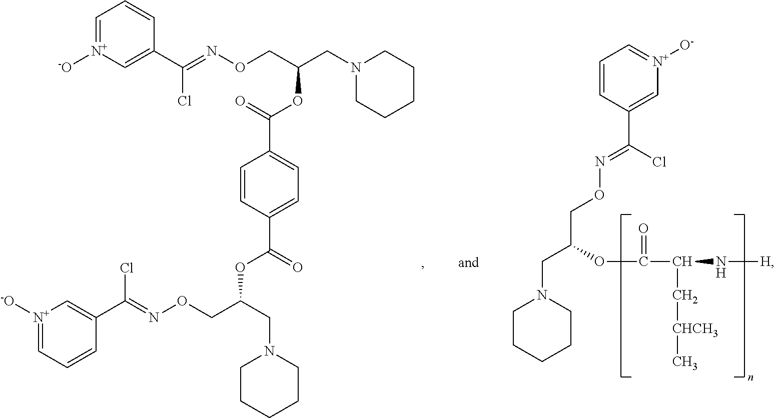

wherein n is 1-50;
or a pharmaceutically acceptable salt thereof.

In a still further embodiment, the present disclosure provides an extended-release composition comprising a therapeutically effective amount of a compound of Formula I, wherein the compound of Formula I is a compound of Formula ID:

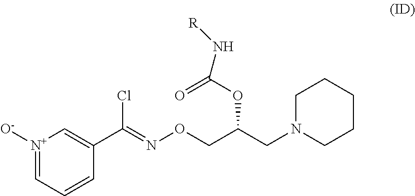

wherein R is selected from the group consisting of, alkenyl, alkenyl, aminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a still further embodiment, the present disclosure provides an extended-release composition comprising a therapeutically effective amount of a compound of Formula ID, wherein the compound of Formula ID is a compound having a structure selected from:

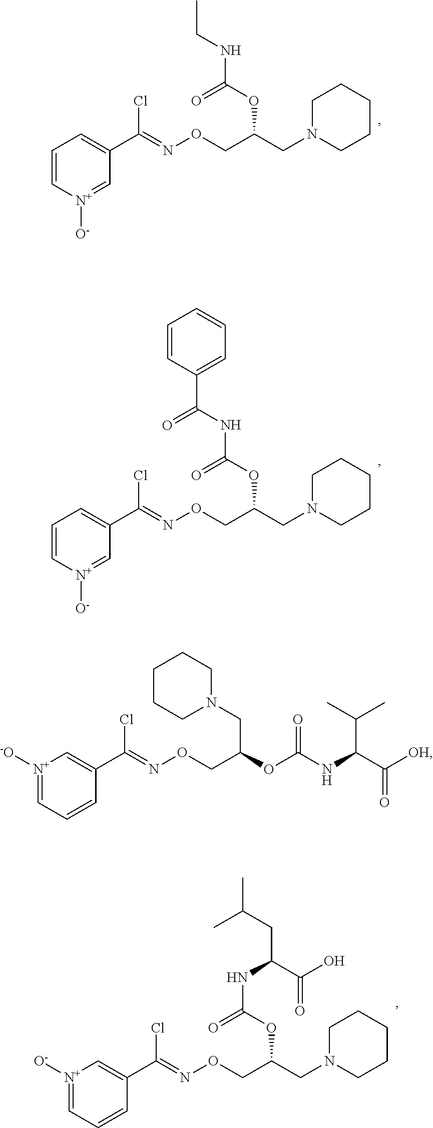

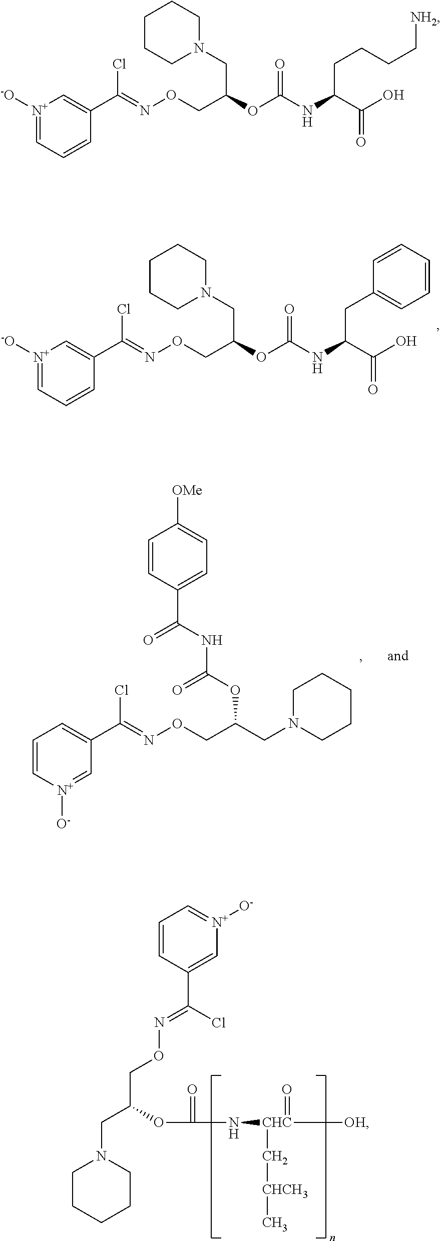

wherein n is 1-50;
or a pharmaceutically acceptable salt thereof.

In a still further embodiment, the present disclosure describes the extended-release composition comprising the therapeutically effective amount of the compound of Formula IC, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In an embodiment, the present disclosure describes an extended-release composition comprising:
a therapeutically effective amount of a compound of Formula II:

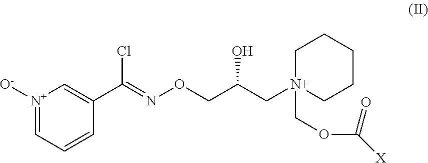

wherein X is selected from the group consisting of R, [O—R], [NR¹R²], and [NRH]
wherein O is oxygen, N is nitrogen, H is hydrogen, and
R, R¹ and R² are independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol
or a pharmaceutically acceptable salt thereof.

In a still further embodiment, the present disclosure provides an extended-release composition comprising a therapeutically effective amount of a compound of Formula II, wherein the compound of Formula II is selected from:

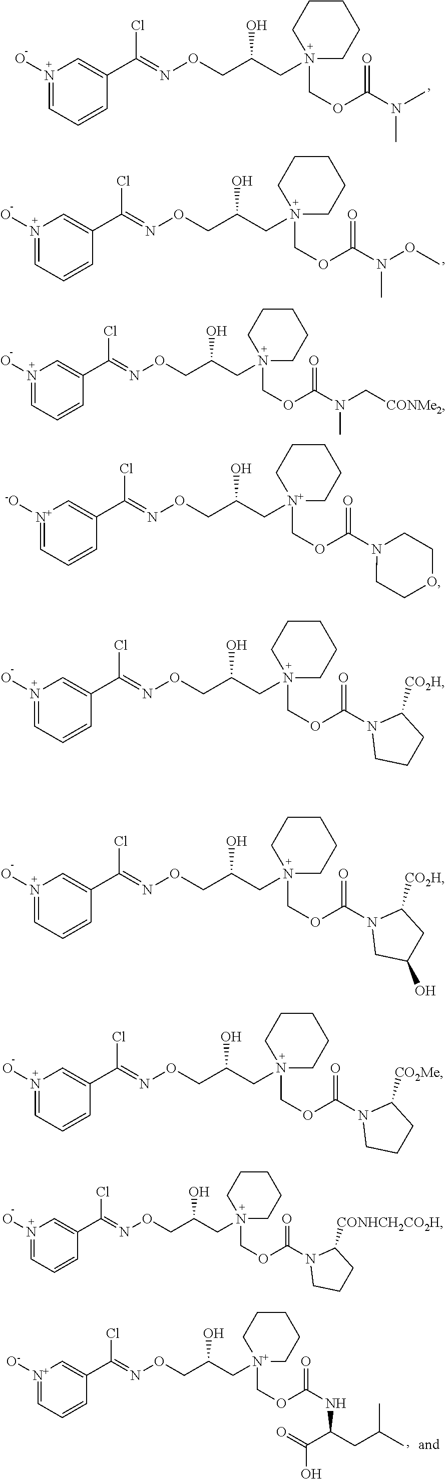

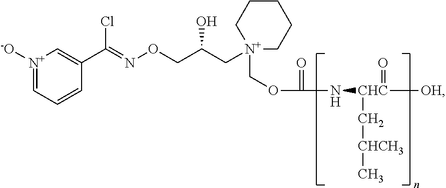

wherein n is 1-50;
or a pharmaceutically acceptable salt thereof.

In a still further embodiment, the present disclosure provides an extended-release composition comprising a therapeutically effective amount of a compound of Formula II, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In an embodiment, the present disclosure provides an extended-release composition comprising a therapeutically effective amount of a compound of Formula III:

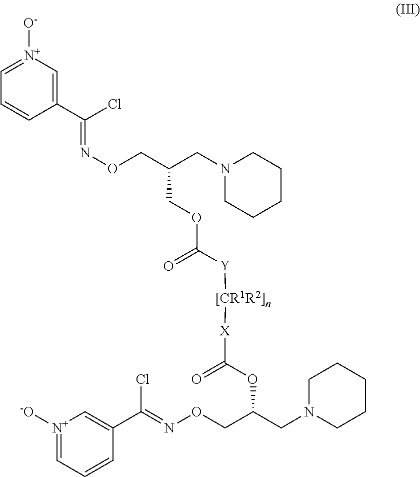

wherein X is selected from the group consisting of O, N, or absent,
Y is selected from the group consisting of O, N, or absent,
n is 1-10,
R¹ and R² are selected independently from each other and for each CR¹R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a further embodiment, the present disclosure provides an extended-release composition comprising a therapeutically effective amount of a compound of Formula III, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In an embodiment, the present disclosure provides an extended-release composition comprising a therapeutically effective amount of a compound of Formula IV:

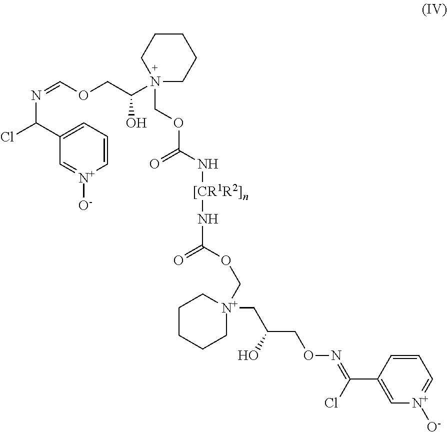

n is 1-10,
R¹ and R² are selected independently from each other and for each CR₁R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a further embodiment, the present disclosure provides an extended-release composition comprising a therapeutically effective amount of a compound of Formula IV, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In an embodiment, the present disclosure provides an extended-release composition comprising a therapeutically effective amount of a compound of Formula V

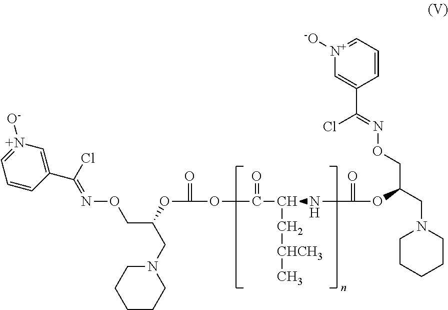

wherein n is 1-50,
or a pharmaceutically acceptable salt thereof.

In a further embodiment, the present disclosure provides an extended-release composition comprising a therapeutically effective amount of a compound of Formula V, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In an embodiment, the present disclosure provides an extended-release composition comprising a therapeutically effective amount of a compound of Formula VI:

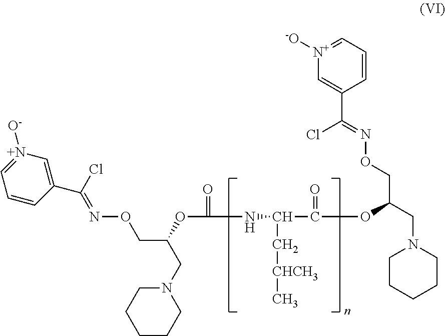

wherein n is 1-50;
or a pharmaceutically acceptable salt thereof.

In a further embodiment, the present disclosure provides an extended-release composition comprising a therapeutically effective amount of a compound of Formula VI, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In an embodiment, the present disclosure provides a composition comprising a therapeutically effective amount of a compound of Formula VII:

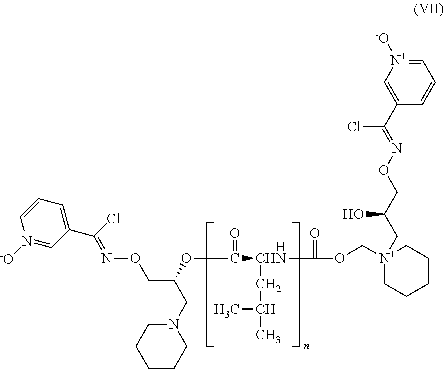

wherein n is 1-50,
or a pharmaceutically acceptable salt thereof.

In certain embodiment, the present disclosure provides an extended-release composition comprising a therapeutically effective amount of a compound of Formula I to Formula VII, wherein the pharmaceutically acceptable salt is selected from sulfate, citrate, acetate, oxalate, chloride, hydrochloride, bromide, hydrobromide, iodide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate. In a still further embodiment, the pharmaceutically acceptable salt is citrate.

In an embodiment, the present disclosure provides an extended-release composition comprising a therapeutically effective amount of a compound having a structure selected from:

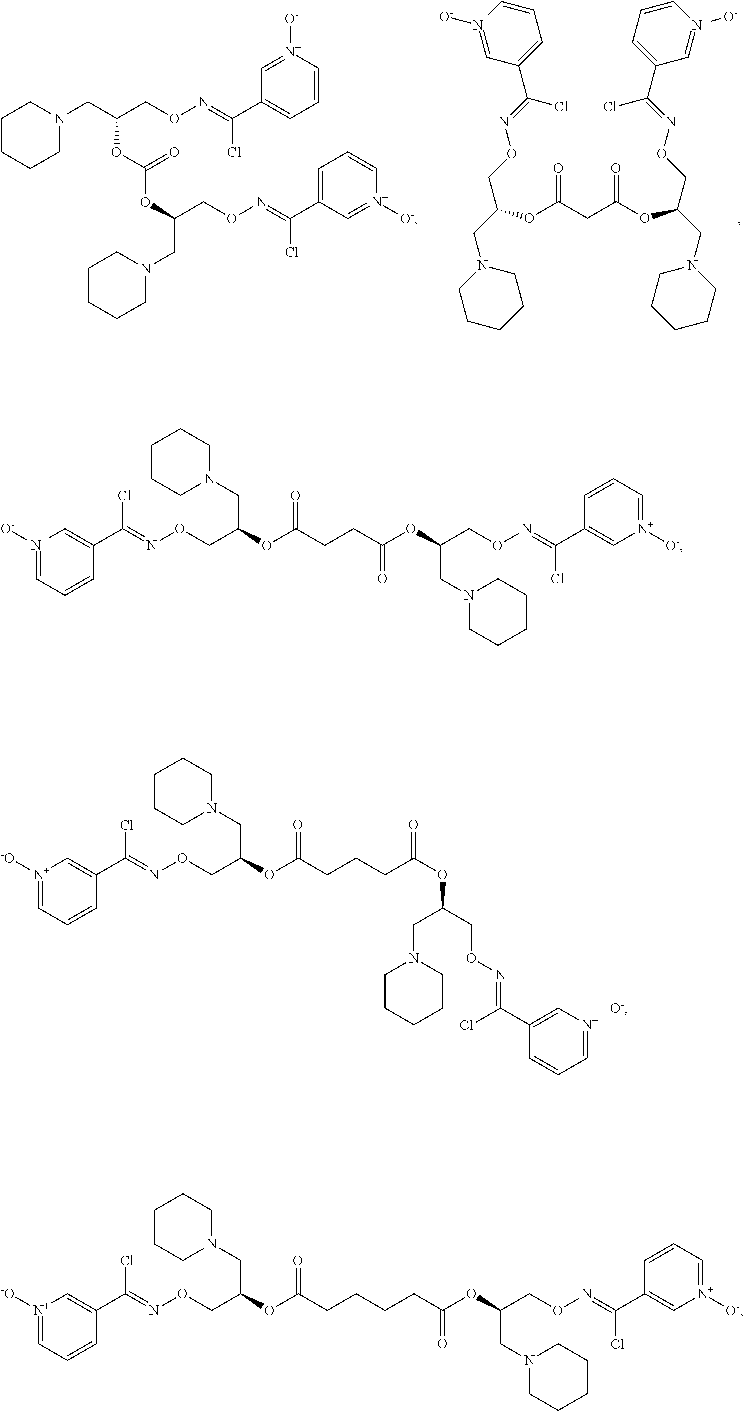

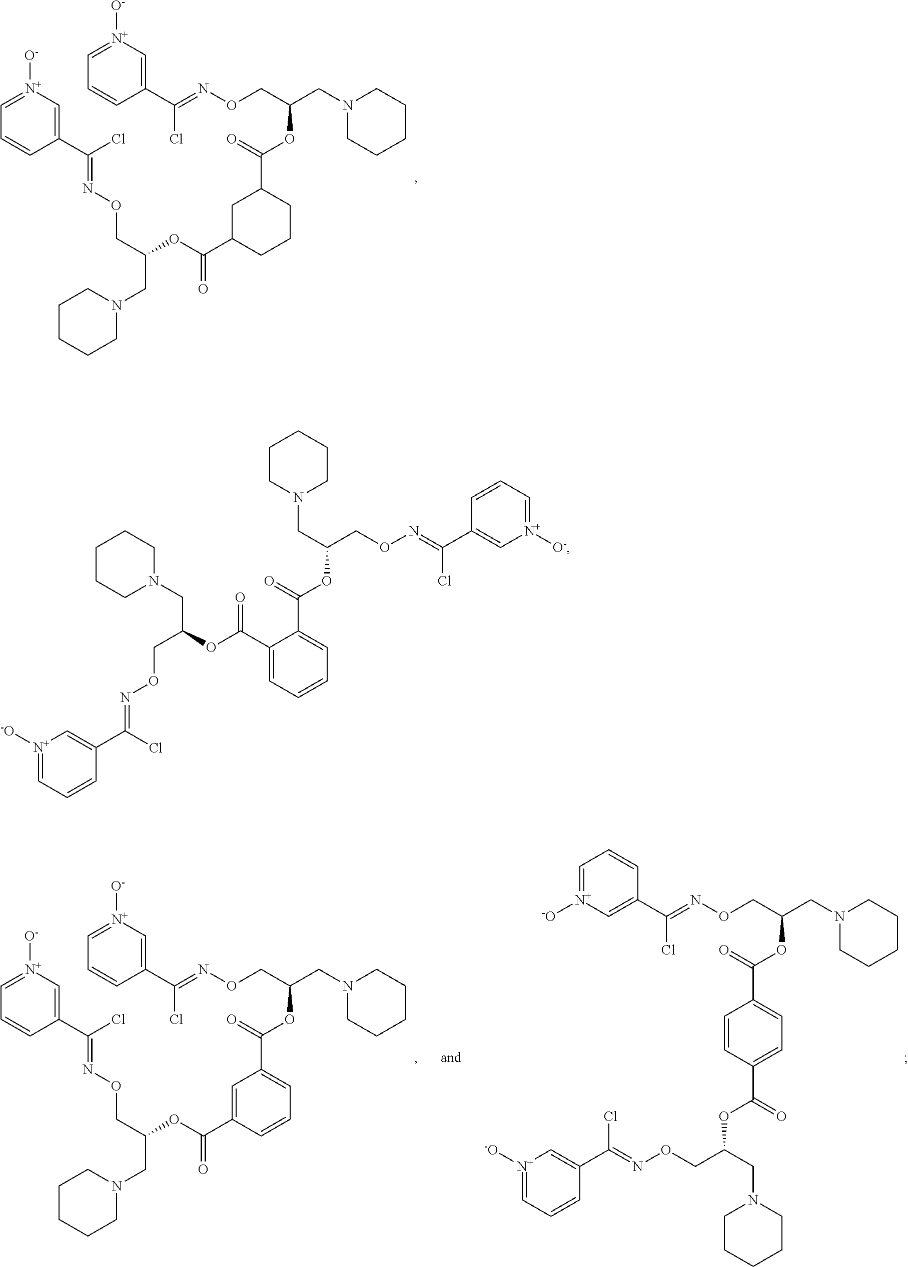

or a pharmaceutically acceptable salt thereof.

In an embodiment, the present disclosure provides the extended release composition comprising the therapeutically effective amount of the compound having a structure selected from

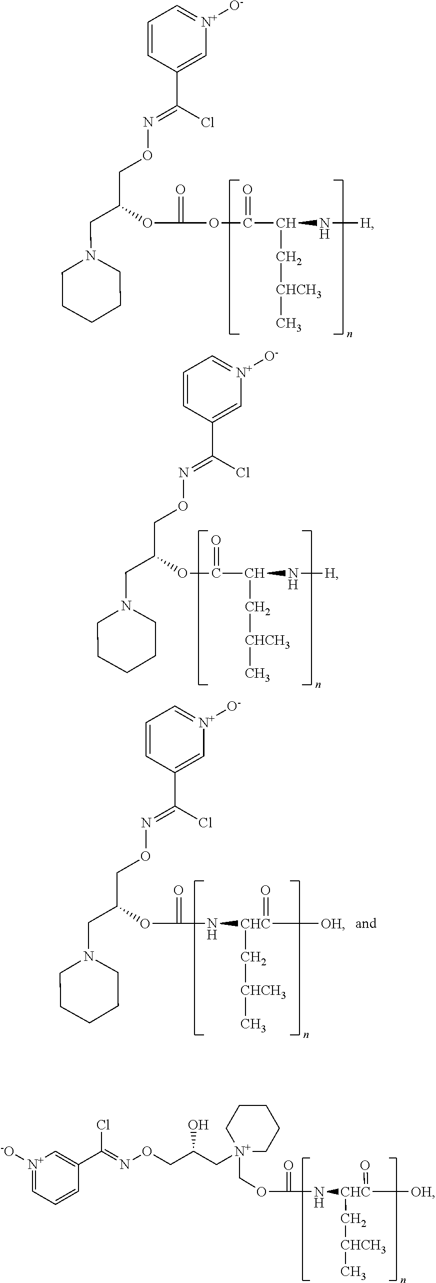

wherein n is 1-50; or a pharmaceutically acceptable salt thereof. Alternatively, n is 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In a still further embodiment, the present disclosure describes the extended-release composition comprising the therapeutically effective amount of the compound of Formula I or Formula II, wherein the first pharmaceutically acceptable salt is a pharmaceutically acceptable salt of the compound of Formula I selected from: sulfate, citrate, acetate, oxalate, chloride, hydrochloride, bromide, hydrobromide, iodide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate. In a further aspect, the pharmaceutically acceptable salt of the compound of Formula I is citrate.

In a further embodiment, the present technology relates to an extended-release composition comprising a therapeutically effective amount of a compound of Formula I or Formula II, wherein the compound of Formula I or Formula II is present as a stereoisomer of said compound. In a still further embodiment, the compound of Formula I or Formula II is present as a stereoisomer and comprises a sub-structure selected from (+)-(R)—N-[2-hydroxy-3-(1-piperidinyl)-propoxy]-pyridine-1-oxide-3-carboximidoyl chloride, and (−)-(S)—N-[2-hydroxy-3-(1-piperidinyl)-propoxy]-pyridine-1-oxide-3-carboximidoyl chloride, (Z)—(R)—N-[2-hydroxy-3-(1-piperidinyl)-propoxy]-pyridine-1-oxide-3-carboximidoyl chloride, (E)-(R)—N-[2-hydroxy-3-(1 piperidinyl)-propoxy]-pyridine-1-oxide-3-carboximidoyl chloride, (Z)—(S)—N-[2-hydroxy-3-(1-piperidinyl)-propoxy]-pyridine-1-oxide-3-carboximidoyl chloride; and (E)-(S)—N-[2-hydroxy-3-(1-piperidinyl)-propoxy]-pyridine-1-oxide-3-carboximidoyl chloride.

In a still further embodiment, the present disclosure describes the extended-release composition comprising the therapeutically effective amount of the compound of Formula I or Formula II, wherein the compound of Formula 1 is present in a form selected from polymorphs, crystals, and combinations thereof.

In a still further embodiment, the present disclosure describes the extended-release composition comprising the therapeutically effective amount of the compound of Formula I or Formula II, wherein the composition further comprises at least one excipient. In a further embodiment, the at least one excipient is selected from antiadherents, binders, coatings, disintegrants, gel forming agents, fillers, flavors and colors, glidants, lubricants, preservatives, sorbents and sweeteners.

In a still further embodiment, the present disclosure describes the extended-release composition comprising the therapeutically effective amount of the compound of Formula I or Formula II, wherein the at least one additional active pharmaceutical ingredient is selected from bimoclomol, miglustat, eliglustat, N-acetyl-L-leucine and 2-hydroxypropyl-β-cyclodextrin, and combinations thereof. In a further embodiment, the at least one additional active pharmaceutical ingredient is selected from miglustat, N-acetyl-L-leucine, and combinations thereof. In a further embodiment, the at least one additional active pharmaceutical ingredient is a combination of miglustat and N-acetyl-L-leucine.

In a still further embodiment, the present disclosure describes the extended-release composition comprising the therapeutically effective amount of the compound of Formula I or Formula II, wherein the composition is formulated for oral administration. In a further embodiment, the composition is a solid oral dosage formulation. In a further embodiment, the solid oral dosage formulation is selected from a sublingual, a gummy, a chewable tablet, a rapidly dissolving tablet, a tablet, a capsule, a caplet, a troche, a lozenge, an oral powder, a solution, a thin strip, an oral thin film (OTF), an oral strip, and a rectal film.

In a still further embodiment, the present disclosure describes the extended-release composition comprising the therapeutically effective amount of the compound of Formula I or Formula II, wherein the composition further comprises a release controlling agent selected from polymer coating, polymer matrix, and combinations thereof. In a further embodiment, the release controlling agent is a polymer coating selected from polyethylene oxides (PEO), hydroxypropyl cellulose (HPC), methylcellulose (MC), hydroxypropyl methylcellulose (HPMC), hydroxyethyl cellulose (HEC), ethylcellulose (EC), and sodium carboxymethyl cellulose (CMC) In a further embodiment, the release controlling agent is a polymer matrix selected from hydroxypropylmethylcellulose (HPMC), ethylcellulose (EC), methylcellulose, hypromellose acetate succinate, hypromellose phthalate, cellulose acetate, glycerin monostearate, glyceryl monooleate, glyceryl palmitate, glyceryl behenate, hydrogenated vegetable oil, guar gum, polyvinyl alcohol, alginates, xanthan gum, carnauba wax, yellow wax, white wax, zein, carrageenan, carbomers and agar In a further embodiment, the release controlling agent is a combination of the polymer coating and the polymer matrix materials listed above.

In a still further embodiment, the present disclosure describes the extended-release composition comprising the therapeutically effective amount of the compound of Formula I or Formula II, wherein the composition is a liquid oral dosage formulation. In a further embodiment, the liquid oral dosage formulation is selected from syrups, emulsions, solutions, and suspensions.

In a still further embodiment, the present disclosure describes the extended-release composition comprising the therapeutically effective amount of the compound of Formula I or Formula II, wherein the therapeutically effective amount of the compound of Formula I ranges from about 1 to about 5000 mg per day. In a still further aspect, the therapeutically effective amount ranges from about 50 mg to about 2500 mg, alternatively about 100 mg to about 2000 mg, alternatively about 200 mg to about 1500 mg, alternatively about 500 mg to about 1000 mg per day.

In a still further embodiment, the present disclosure describes the extended-release composition comprising the therapeutically effective amount of the compound of Formula I or Formula II, wherein the therapeutically effective amount of the compound of Formula I is administered as a unit dose. In a still further embodiment, the therapeutically effective amount is administered in 1 unit dose.

In certain embodiments, the present disclosure provides for the extended-release composition comprising a therapeutically effective amount of a compound described above, or a pharmaceutically acceptable salt thereof; wherein the extended release composition further comprises a therapeutically effective amount of at least one additional active pharmaceutical ingredient designed to release immediately upon administration. In certain embodiments, the at least one additional active pharmaceutical ingredient is selected from arimoclomol, bimoclomol, miglustat, eligustat, and N-acetyl-L-leucine.

### III. Methods of Treating Neurodegenerative or Metabolic Disorders Using Arimoclomol Prodrugs

In one embodiment, the present technology provides a method of treating a neurodegenerative or metabolic disease or disorder in a patient in need thereof, the method comprising:
administering to the patient in need thereof a composition comprising a therapeutically effective amount of a compound having a structure of Formula I:

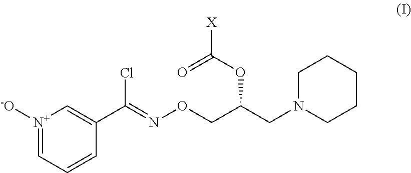

wherein X is selected from the group consisting of R, [O—R], [NR¹R²], and [NRH]
wherein O is oxygen, N is nitrogen, H is hydrogen, and
R, R¹ and R² are independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol,
or a pharmaceutically acceptable salt thereof.

In a further embodiment, the present disclosure provides method of treating a neurodegenerative or metabolic disease or disorder, the method comprising administering to the patient in need thereof a composition comprising a therapeutically effective amount of a compound of Formula I, wherein the compound of Formula I is a compound having a structure of Formula IA:

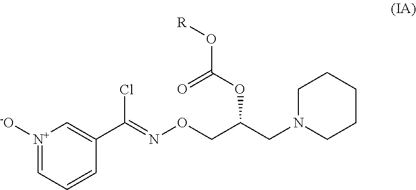

wherein R is selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol.

In a further embodiment, the present disclosure provides method of treating a neurodegenerative or metabolic disease or disorder, the method comprising administering to the patient in need thereof a composition comprising a therapeutically effective amount of a compound of Formula IA, wherein the compound of Formula IA is a compound having a structure selected from:

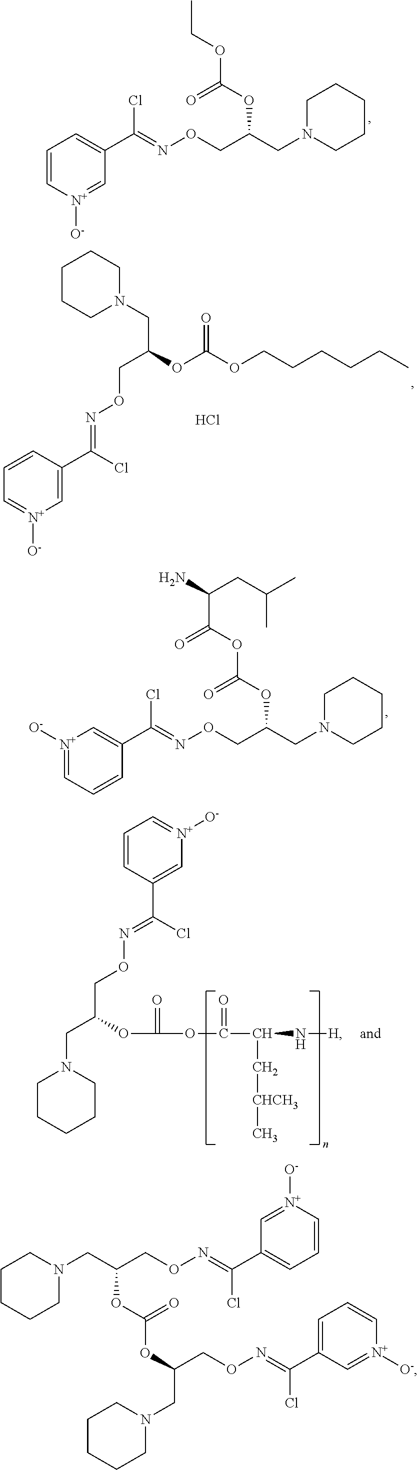

wherein n is 1-50;
or a pharmaceutically acceptable salt thereof.

In a further embodiment, the present disclosure provides method of treating a neurodegenerative or metabolic disease or disorder, the method comprising administering to the patient in need thereof a composition comprising a therapeutically effective amount of a compound of Formula IA, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In a further embodiment, the present disclosure provides method of treating a neurodegenerative or metabolic disease or disorder, the method comprising administering to the patient in need thereof a composition comprising a therapeutically effective amount of a compound of Formula I, wherein the compound of Formula I is a compound having a structure of Formula IB:

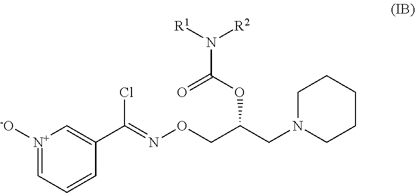

wherein R¹ and R² are independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol; or
or a pharmaceutically acceptable salt thereof.

In a further embodiment, the present disclosure provides method of treating a neurodegenerative or metabolic disease or disorder, the method comprising administering to the patient in need thereof a composition comprising a therapeutically effective amount of a compound of Formula IB, wherein the compound of Formula IB is a compound having a structure selected from:

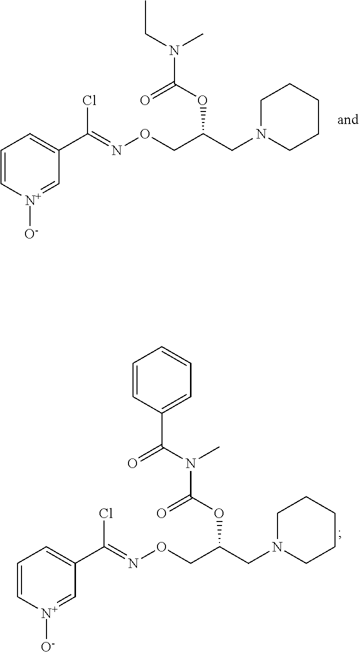 [SMILES: CCN(C)C(=O)O[C@@H](CO/N=C(\Cl)c1ccc[n+]([O-])c1)CN1CCCCC1 ; CN(C(=O)O[C@@H](CO/N=C(\Cl)c1ccc[n+]([O-])c1)CN1CCCCC1)C(=O)c1ccccc1]

or a pharmaceutically acceptable salt thereof.

In a further embodiment, the present disclosure provides method of treating a neurodegenerative or metabolic disease or disorder, the method comprising administering to the patient in need thereof a composition comprising a therapeutically effective amount of a compound of Formula I, wherein the compound of Formula I is a compound having a structure of Formula IC:

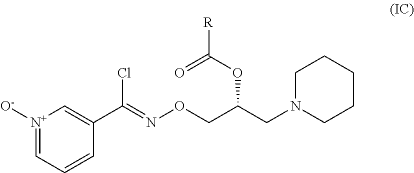

wherein R is selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a further embodiment, the present disclosure provides method of treating a neurodegenerative or metabolic disease or disorder, the method comprising administering to the patient in need thereof a composition comprising a therapeutically effective amount of a compound of Formula IC, wherein the compound of Formula IC is a compound having a structure selected from:

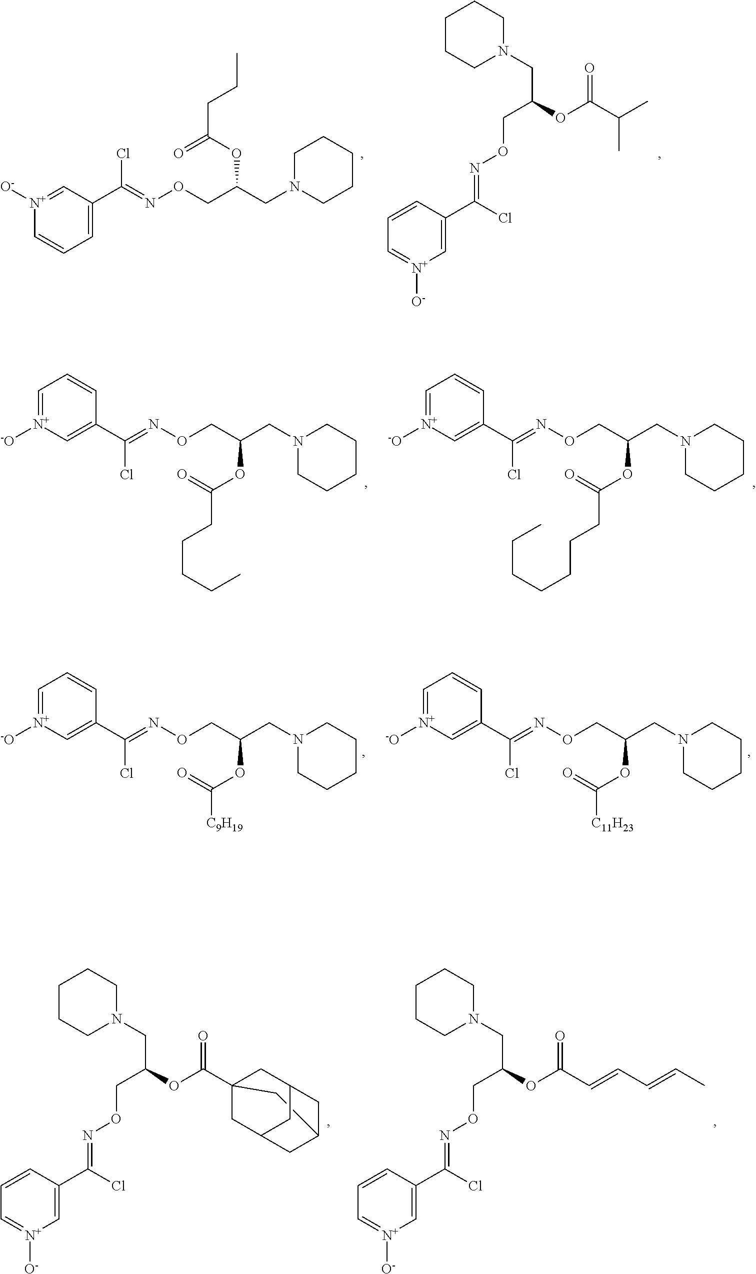

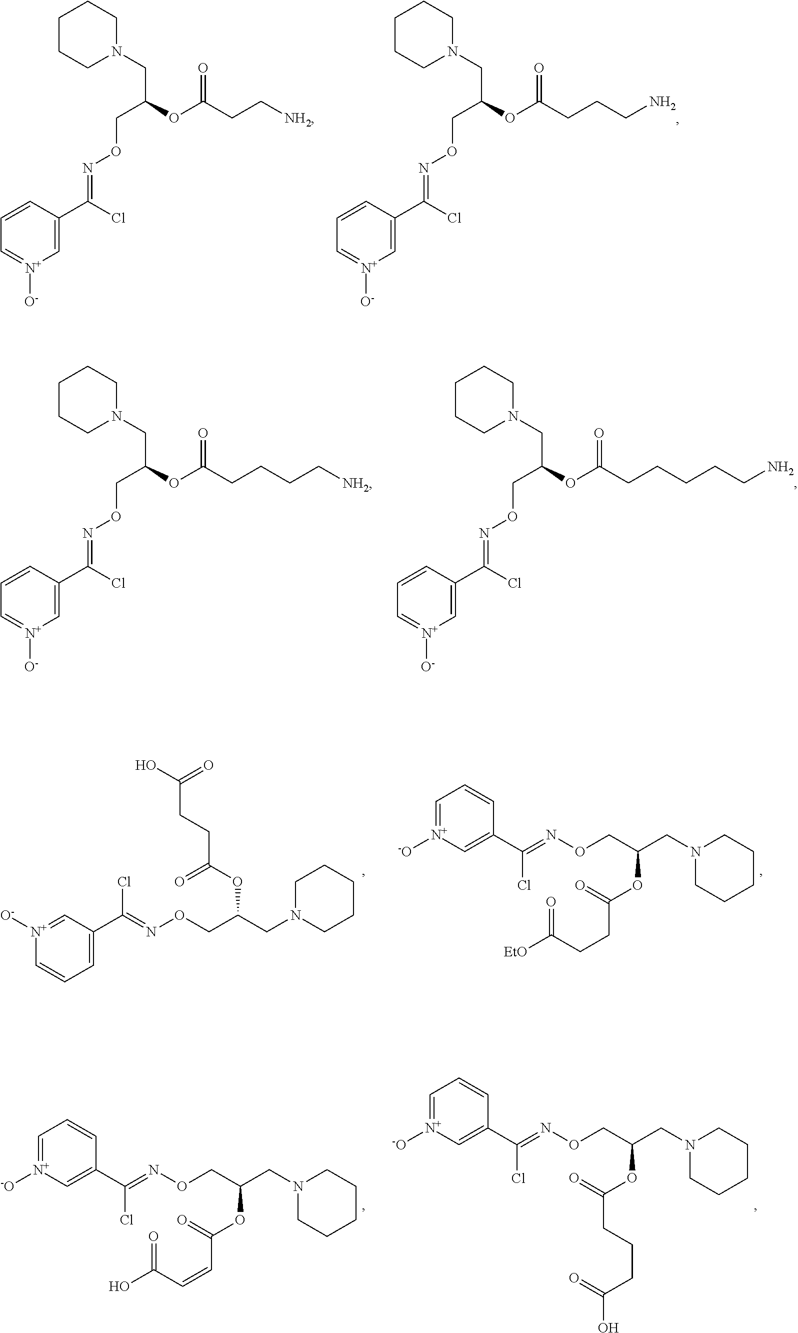

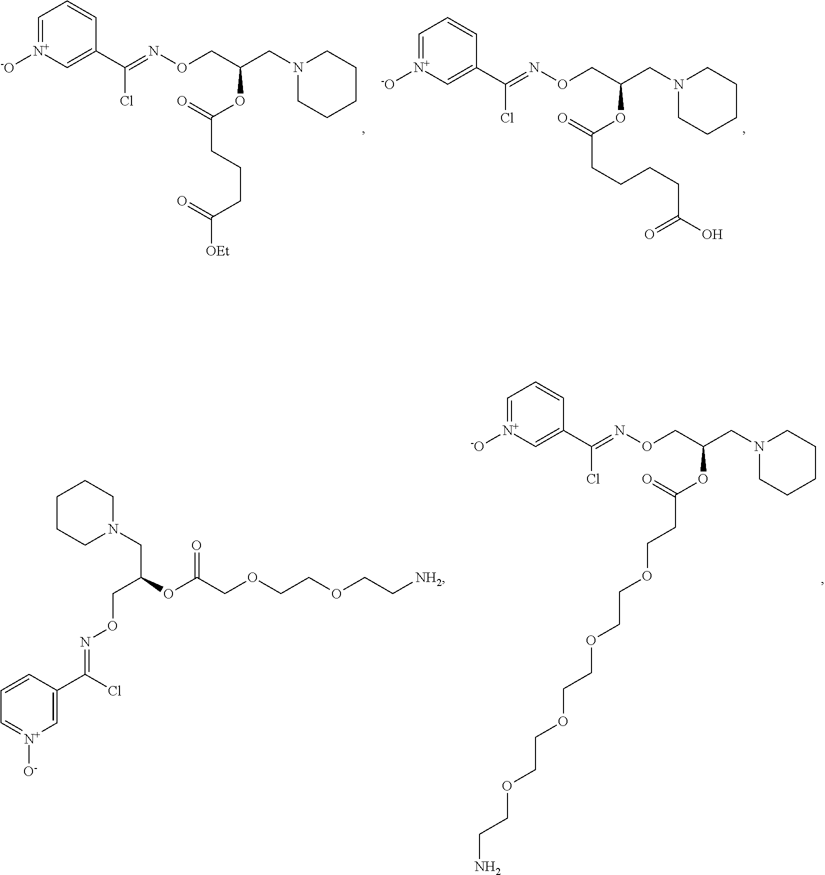

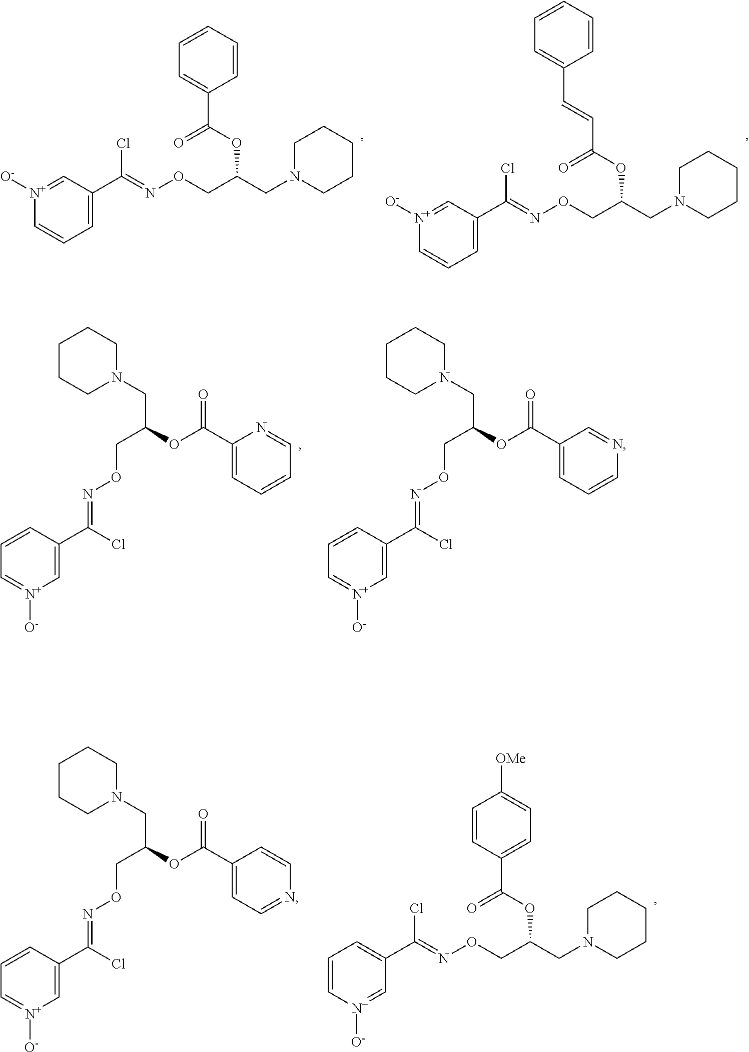

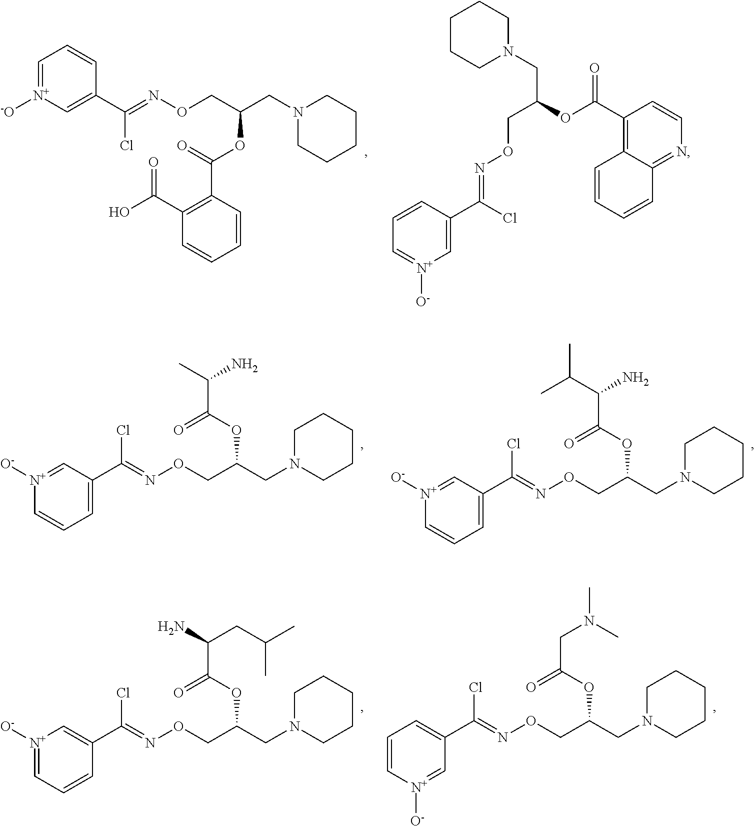

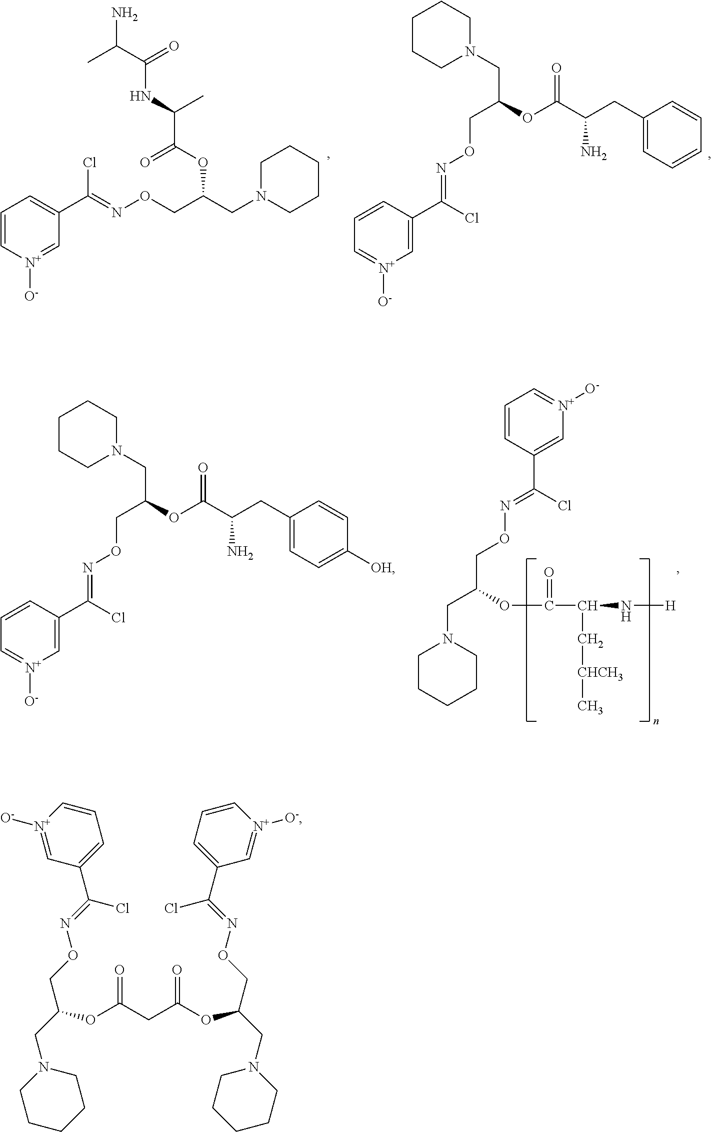

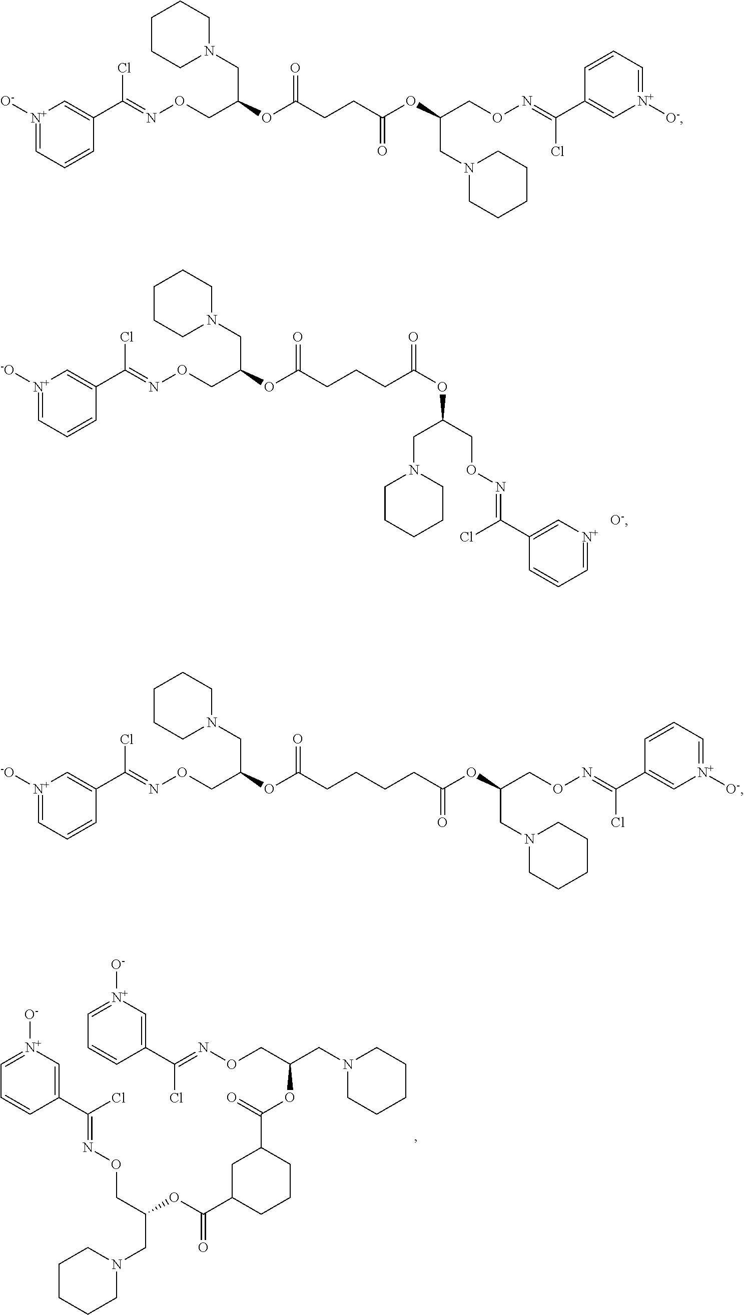

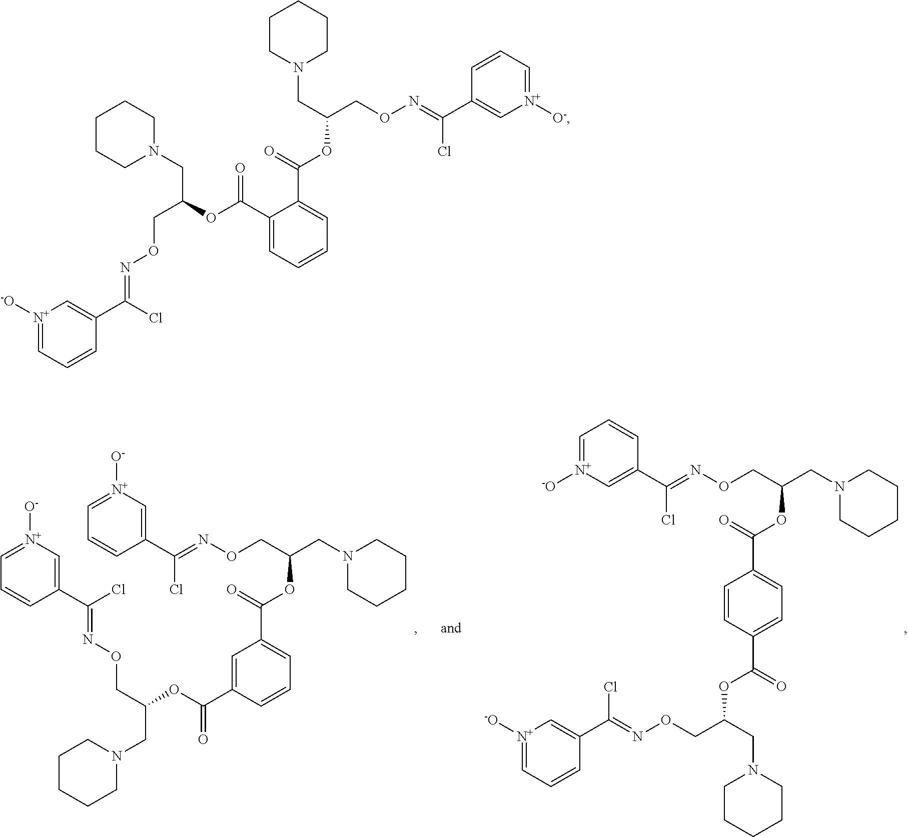

wherein n is 1-50;
or a pharmaceutically acceptable salt thereof.

In a further embodiment, the present disclosure provides method of treating a neurodegenerative or metabolic disease or disorder, the method comprising administering to the patient in need thereof a composition comprising a therapeutically effective amount of a compound of Formula IC, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In a further embodiment, the present disclosure provides method of treating a neurodegenerative or metabolic disease or disorder, the method comprising administering to the patient in need thereof a composition comprising a therapeutically effective amount of a compound of Formula IC, the method comprising administering to the patient in need thereof a therapeutically effective amount of the compound of Formula IC, wherein the compound of Formula IC is a compound having a structure selected from:

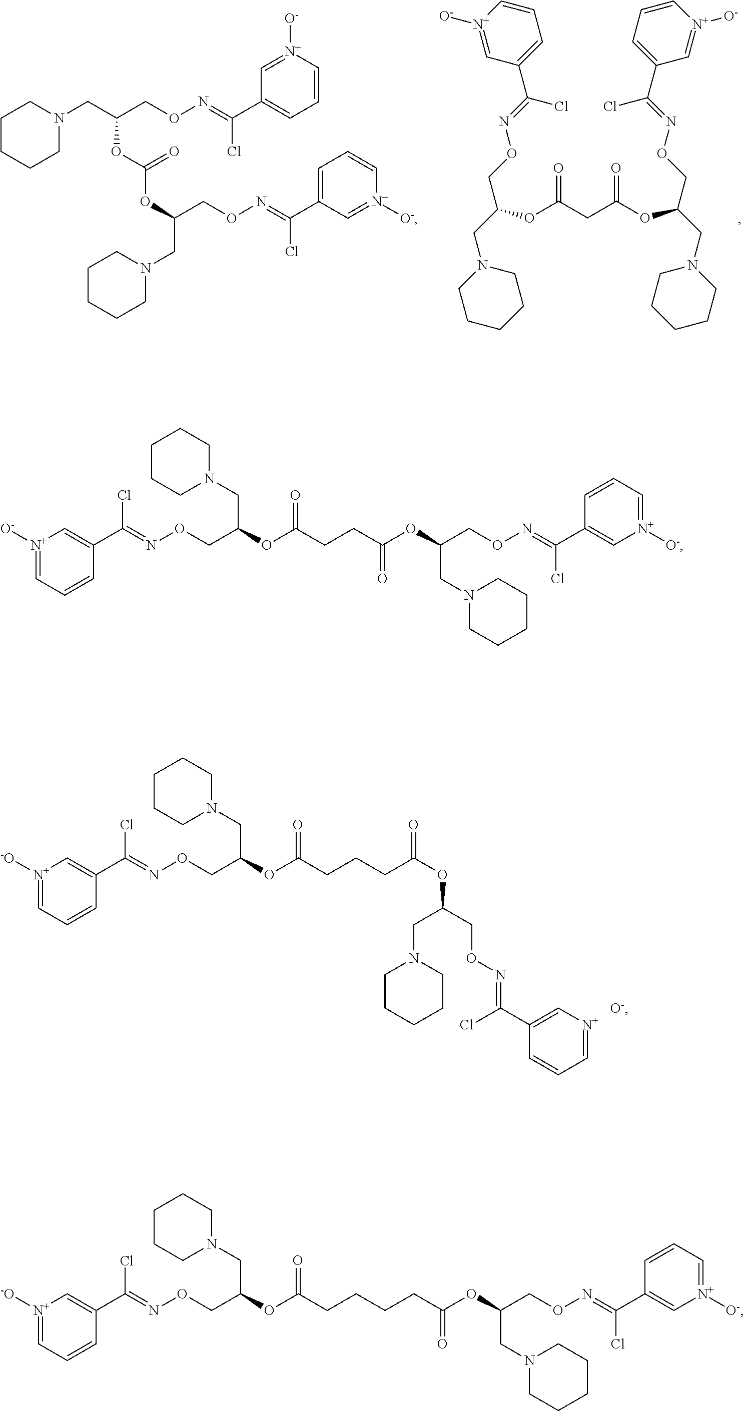

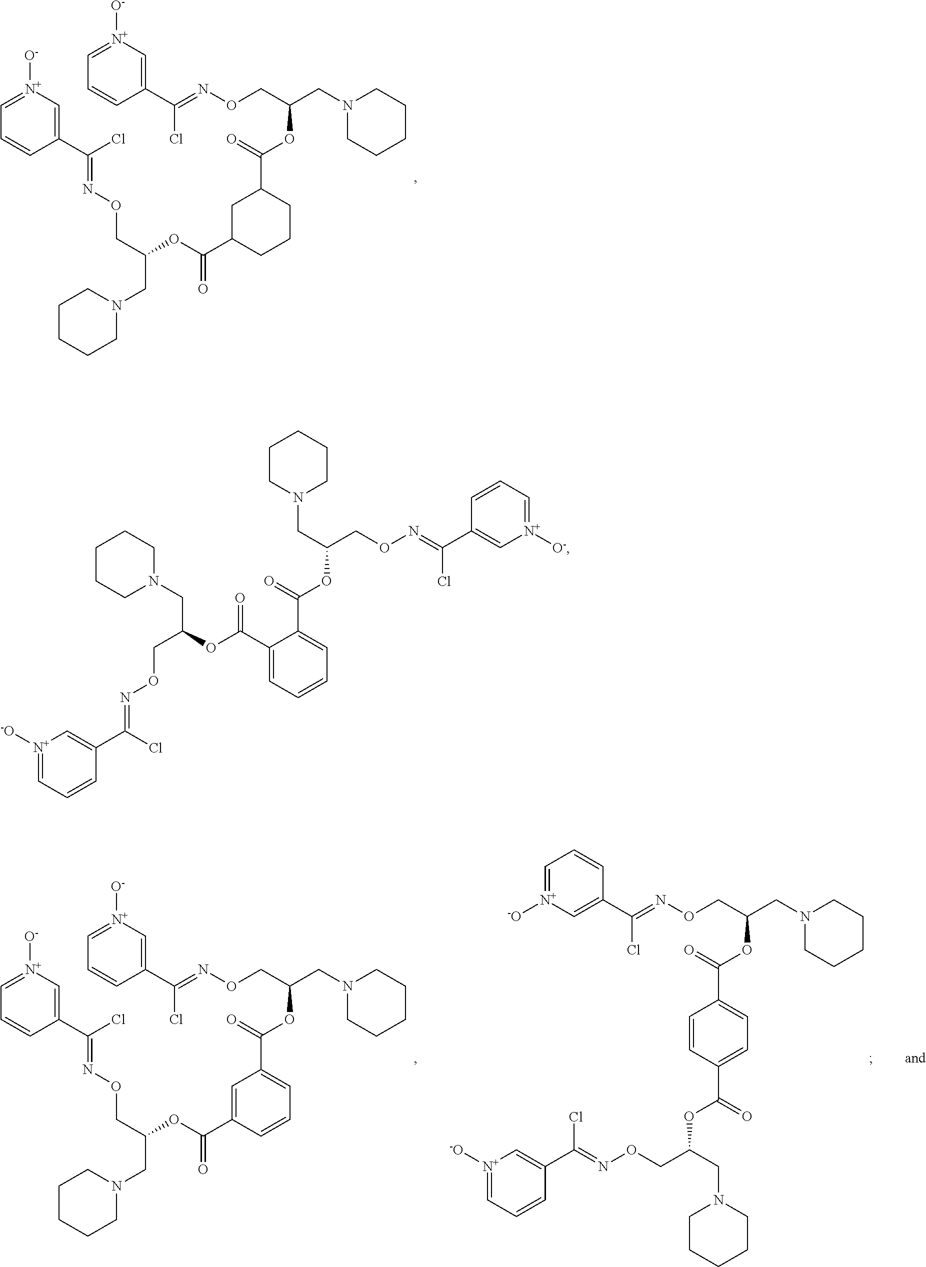

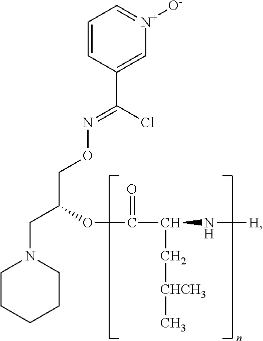

wherein n is 1-50;
or a pharmaceutically acceptable salt thereof.

In a further embodiment, the present disclosure provides method of treating a neurodegenerative or metabolic disease or disorder, the method comprising administering to the patient in need thereof a composition comprising a therapeutically effective amount of a compound of Formula I, wherein the compound of Formula I is a compound having a structure of Formula ID:

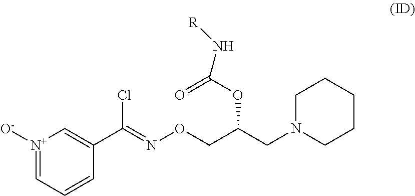

wherein R is selected from the group consisting of, alkenyl, alkenyl, aminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a further embodiment, the present disclosure provides method of treating a neurodegenerative or metabolic disease or disorder, the method comprising administering to the patient in need thereof a composition comprising a therapeutically effective amount of a compound of Formula ID, wherein the compound of Formula ID is a compound having a structure selected from:

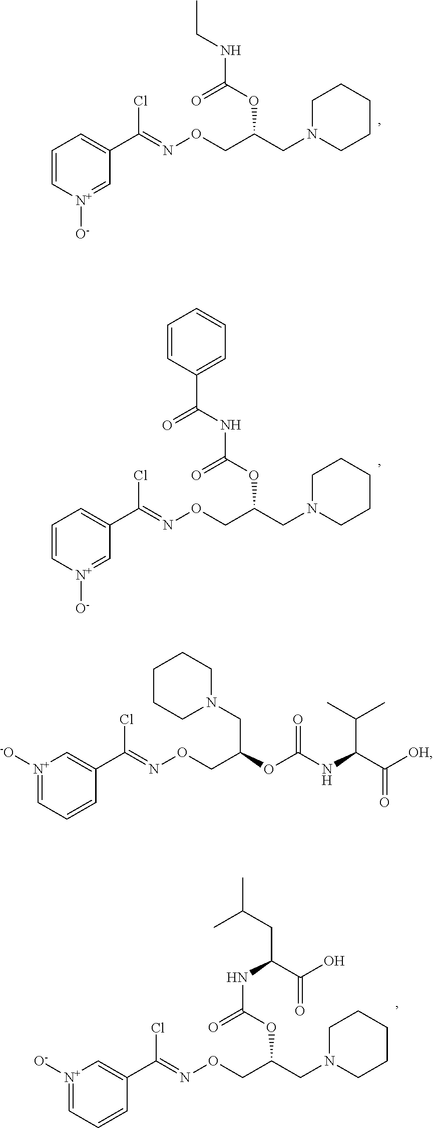

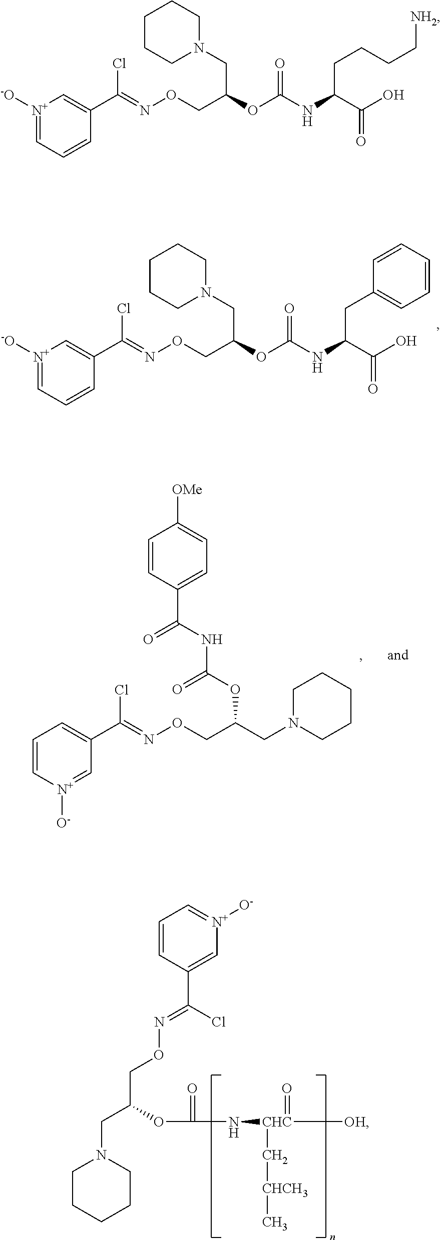

wherein n is 1-50;
or a pharmaceutically acceptable salt thereof.

In a further embodiment, the present disclosure provides method of treating a neurodegenerative or metabolic disease or disorder, the method comprising administering to the patient in need thereof a composition comprising a therapeutically effective amount of a compound of Formula ID, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In an embodiment, the present disclosure describes a method of treating a neurodegenerative or metabolic disorder in a patient in need thereof, the method comprising:
administering to the patient in need thereof a therapeutically effective amount of a compound of Formula II:

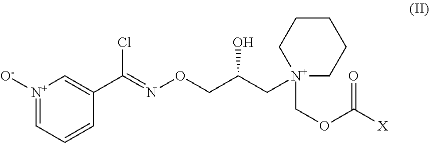

wherein R¹ and R² are independently selected from alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a further embodiment, the present disclosure provides method of treating a neurodegenerative or metabolic disease or disorder, the method comprising administering to the patient in need thereof a composition comprising a therapeutically effective amount of a compound of Formula II, wherein the compound of Formula II has a structure selected from:

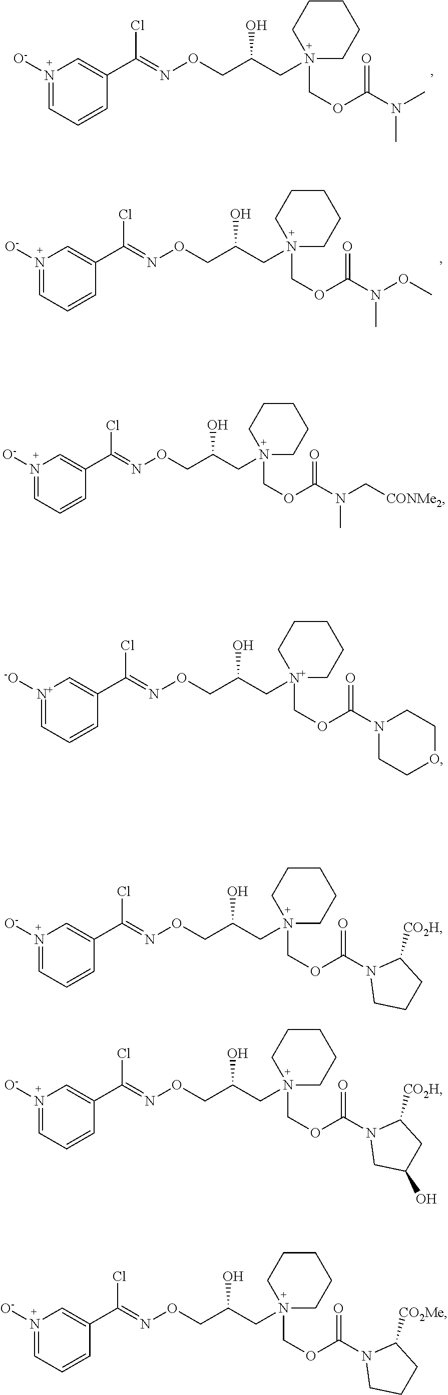

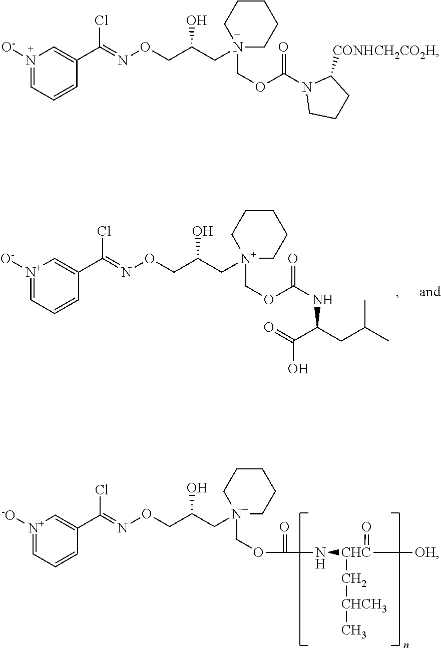

wherein n is 1-50;
or a pharmaceutically acceptable salt thereof.

In a further embodiment, the present disclosure provides method of treating a neurodegenerative or metabolic disease or disorder, the method comprising administering to the patient in need thereof a composition comprising a therapeutically effective amount of a compound of Formula II, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In an embodiment, the present disclosure provides method of treating a neurodegenerative or metabolic disease or disorder, the method comprising administering to the patient in need thereof a composition comprising a therapeutically effective amount of a compound of Formula III:

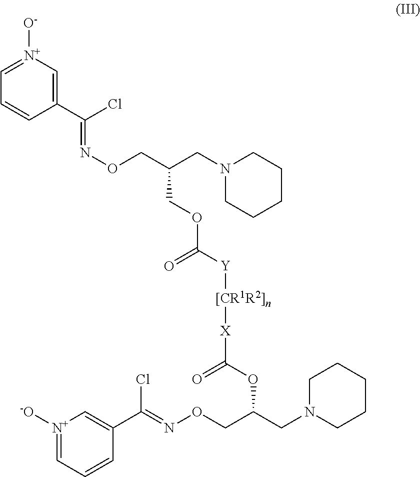

wherein X is selected from the group consisting of O, N, or absent,
Y is selected from the group consisting of O, N, or absent,
n is 1-10,
R¹ and R² are selected independently from each other and for each CR¹R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a further embodiment, the present disclosure provides method of treating a neurodegenerative or metabolic disease or disorder, the method comprising administering to the patient in need thereof a composition comprising a therapeutically effective amount of a compound of Formula III, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In an embodiment, the present disclosure provides method of treating a neurodegenerative or metabolic disease or disorder, the method comprising administering to the patient in need thereof a composition comprising a therapeutically effective amount of a compound of Formula IV:

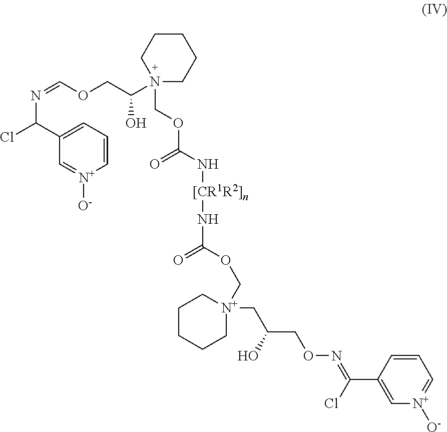

n is 1-10,
R¹ and R² are selected independently from each other and for each CR₁R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

In a further embodiment, the present disclosure provides method of treating a neurodegenerative or metabolic disease or disorder, the method comprising administering to the patient in need thereof a composition comprising a therapeutically effective amount of a compound of Formula IV, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In an embodiment, the present disclosure provides method of treating a neurodegenerative or metabolic disease or disorder, the method comprising administering to the patient in need thereof a composition comprising a therapeutically effective amount of a compound of Formula V

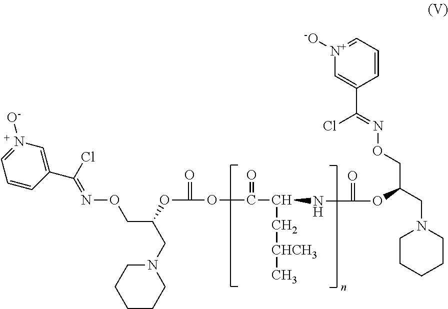

wherein n is 1-50,
or a pharmaceutically acceptable salt thereof.

In a further embodiment, the present disclosure provides method of treating a neurodegenerative or metabolic disease or disorder, the method comprising administering to the patient in need thereof a composition comprising a therapeutically effective amount of a compound of Formula V, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In an embodiment, the present disclosure provides method of treating a neurodegenerative or metabolic disease or disorder, the method comprising administering to the patient in need thereof a composition comprising a therapeutically effective amount of a compound of Formula VI:

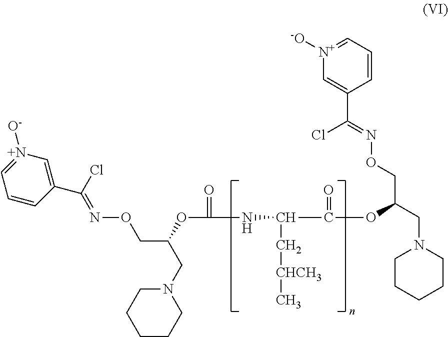

wherein n is 1-50;
or a pharmaceutically acceptable salt thereof.

In a further embodiment, the present disclosure provides method of treating a neurodegenerative or metabolic disease or disorder, the method comprising administering to the patient in need thereof a composition comprising a therapeutically effective amount of a compound of Formula VI, wherein n is alternatively 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In an embodiment, the present disclosure provides method of treating a neurodegenerative or metabolic disease or disorder, the method comprising administering to the patient in need thereof a composition comprising a therapeutically effective amount of a compound of Formula VII:

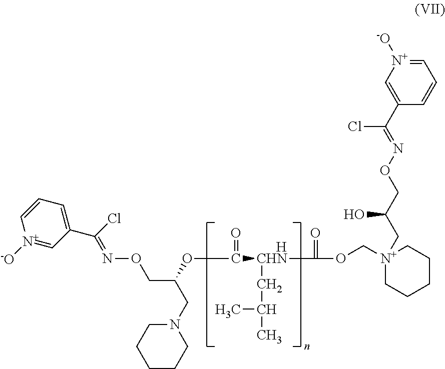

wherein n is 1-50,
or a pharmaceutically acceptable salt thereof.

In certain embodiment, the present disclosure provides method of treating a neurodegenerative or metabolic disease or disorder, the method comprising administering to the patient in need thereof a composition comprising a therapeutically effective amount of a compound of Formula I to Formula VII, wherein the pharmaceutically acceptable salt is selected from sulfate, citrate, acetate, oxalate, chloride, hydrochloride, bromide, hydrobromide, iodide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate. In a still further embodiment, the pharmaceutically acceptable salt is citrate.

In a further embodiment, the present disclosure describes the method of treating a neurodegenerative or metabolic disease or disorder, the method comprising administering to the patient in need thereof a composition comprising a therapeutically effective amount of a compound having a structure selected from:

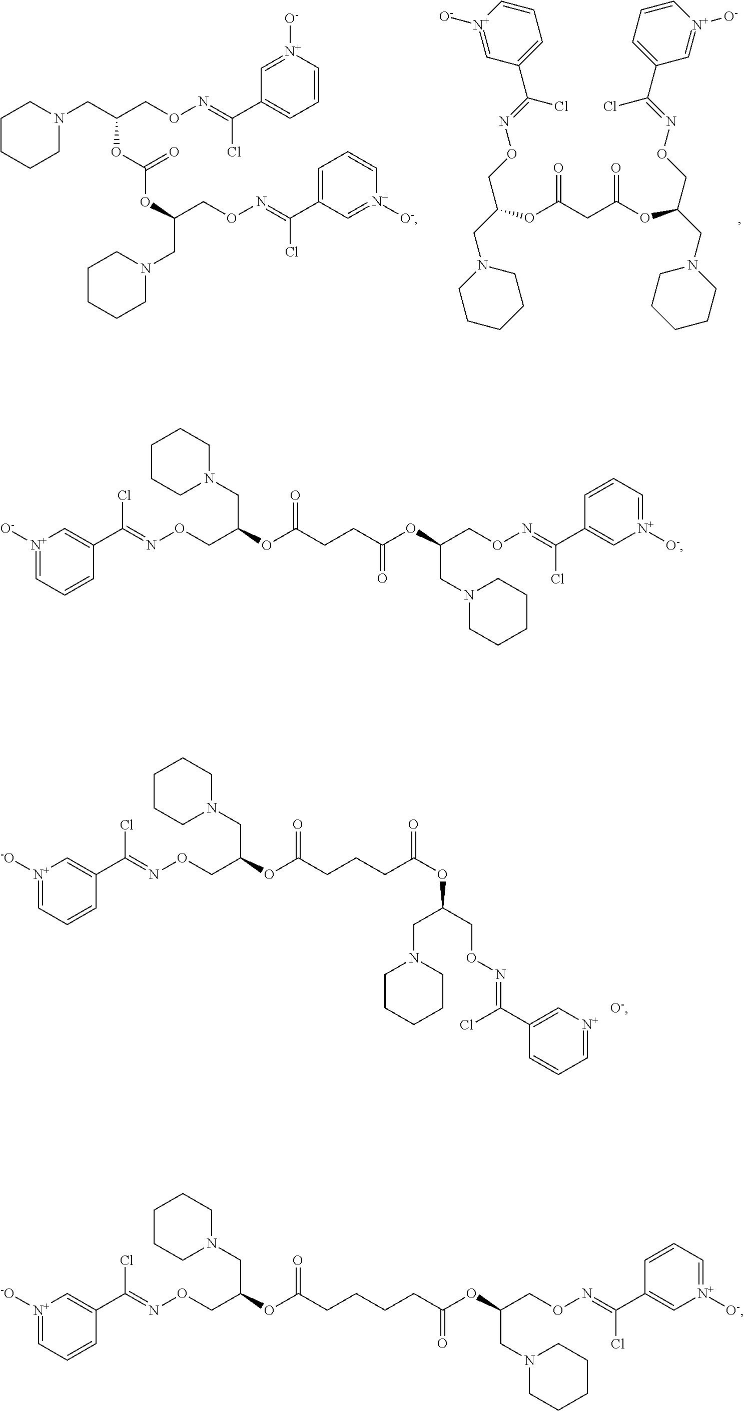

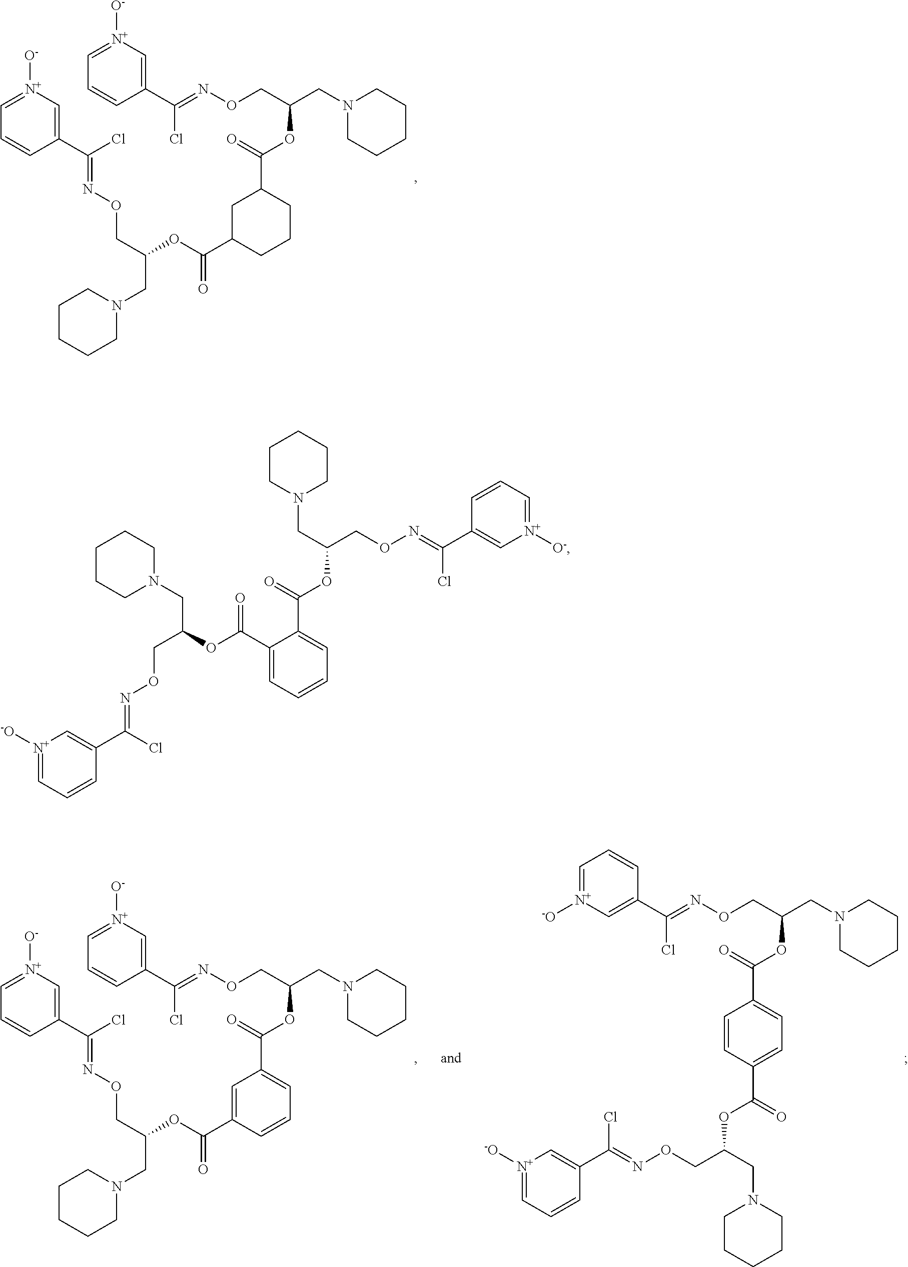

or a pharmaceutically acceptable salt thereof.

In a further embodiment, the present disclosure describes the method of treating a neurodegenerative or metabolic disease or disorder, the method comprising administering to the patient in need thereof the composition comprising the therapeutically effective amount of a compound having a structure selected from

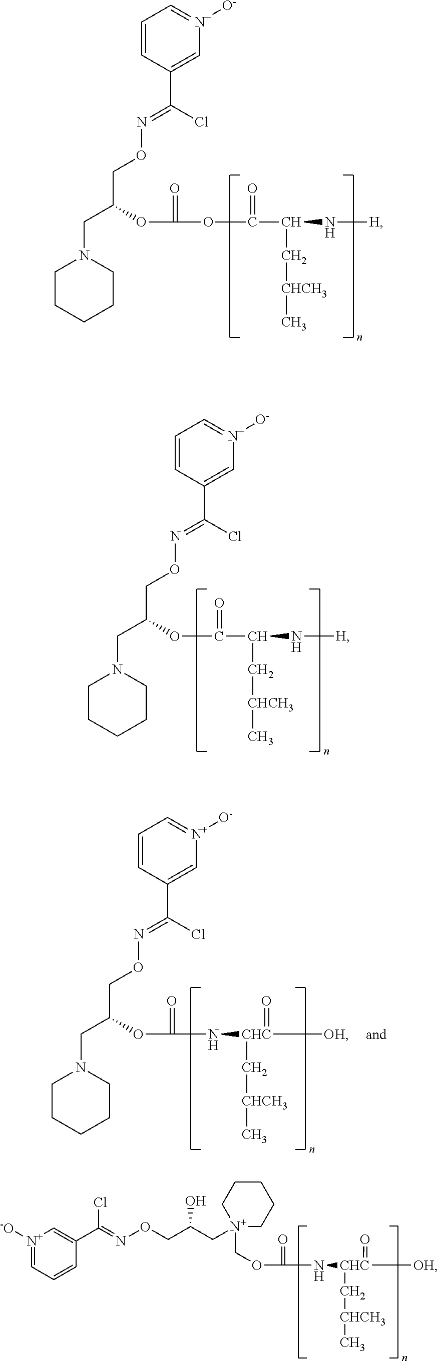

wherein n is 1-50; or a pharmaceutically acceptable salt thereof. Alternatively, n is 1-10, alternatively 11-20, alternatively 21-30, alternatively 31-40, alternatively 41-50, alternatively 1-12, alternatively 5-15, alternatively 8-17, alternatively 13-22, alternatively 19-27, alternatively 25-38, alternatively 33-49, alternatively 44-50.

In a further embodiment, the present disclosure provides a method of treating a neurodegenerative or metabolic disease or disorder, the method comprising administering to the patient in need thereof a composition comprising a therapeutically effective amount of a compound of Formula I to Formula VII, wherein the pharmaceutically acceptable salt is selected from: sulfate, citrate, acetate, oxalate, chloride, hydrochloride, bromide, hydrobromide, iodide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate. In a further embodiment, the pharmaceutically acceptable salt is citrate.

In a further embodiment, the present disclosure provides a method of treating a neurodegenerative or metabolic disease or disorder, the method comprising administering to the patient in need thereof a composition comprising a therapeutically effective amount of a compound of Formula I to Formula VII, wherein the neurodegenerative disease, or metabolic disorder is a lysosomal storage disorder.

“Lysosomal storage disorder” or “Lysosomal Storage Disorders” as it related to the present technology means a disease or disorder associated with reduced lysosomal function. Lysosomal storage diseases (LSDs) are a group of approximately 40 rare inherited metabolic disorders that result from defects in lysosomal function. LSDs are caused by lysosomal dysfunction usually as a consequence of deficiency of a single enzyme required for the metabolism of lipids, glycoproteins or mucopolysaccharides. Although each disorder results from different gene mutations that translate into a deficiency in enzyme activity, they all share a common biochemical characteristic—all lysosomal disorders originate from an abnormal accumulation of substances inside the lysosome. Individually LSDs occur with incidences of less than 1:100000, however, as a group the incidence is about 1:5000-1:10000. Most of these disorders are autosomal recessively inherited, however a few are X-linked recessively inherited, such as Fabry disease.

The lysosomal storage diseases are generally classified by the nature of the primary stored material involved, and can be broadly broken into the following: lipid storage disorders (or lipidoses), mainly sphingolipidoses (including Gaucher's and Niemann-Pick diseases); gangliosidosis (including Tay-Sachs disease); leukodystrophies; mucopolysaccharidoses (including Hunter syndrome and Hurler disease); glycoprotein storage disorders (glycoproteinosis); and nucolipidoses. Depending on the severity of the disease, patients either die at a young and unpredictable age, many within a few months or years of birth, whereas others survive into early adulthood finally succumbing to the various pathologies of their particular disorder. The symptoms of LSD vary, depending on the particular disorder and can be mild to severe. They can include developmental delay, movement disorders, seizures, dementia, deafness and/or blindness. Some people with LSD have enlarged livers (hepatomegaly) and enlarged spleens (splenomegaly), pulmonary and cardiac problems, and abnormal bone growth. The majority of patients are initially screened by an enzyme assay, which is the most efficient method to arrive at a definitive diagnosis. In some families where the disease-causing mutation(s) is known and in certain genetic isolates, mutation analysis may be performed. As there may be numerous different mutations, sequencing of the gene encoding the particular affected enzyme is sometimes necessary to confirm the diagnosis. Prenatal diagnosis may be useful when there is a known genetic risk factor. FEB and TFE3 activate expression of genes responsible for lysosomal protein synthesis. Lysosomes are organelles responsible for removing waste from the cell. Genes activated by TFEB and TFE3 are collectively known as the CLEAR network of genes that include NPC1. In cells unaffected by NPC, normally assembled and maturated proteins migrate to the lysosomal membrane where they play a critical role in transporting cholesterol and other lipids out of the lysosome. In healthy cells, this process supports elimination of cellular waste (autophagy) and promotes healthy neuronal function.

In a further embodiment, the present disclosure provides a method of treating a neurodegenerative or metabolic disease or disorder, the method comprising administering to the patient in need thereof a composition comprising a therapeutically effective amount of a compound of Formula I to Formula VII, wherein the lysosomal storage disorder is selected from sphingolipidoses, mucopolysaccharidoses (MPS), lysosomal glycogen storage disorders, oligosaccharidoses, mucolipidoses, lipid storage disorders, glycoproteinoses, neuronal ceroid lipofuscinoses (NCL), peroxisomal biogenesis disorders (Zellweger spectrum disorders), and other lysosomal storage disorders.

In a further embodiment, the present disclosure provides a method of treating a neurodegenerative or metabolic disease or disorder, the method comprising administering to the patient in need thereof a composition comprising a therapeutically effective amount of a compound of Formula I to Formula VII, wherein the sphingolipidosis is selected from Gaucher disease type 1, Gaucher disease type 2, Gaucher disease type 3, Fabry disease, Niemann-pick disease type A, Niemann-pick disease type B, Niemann-pick disease type C, Tay-Sachs disease, Sandhoff disease, GM1 gangliosidosis, metachromatic leukodystrophy (MLD), Krabbe disease, and Farber lipogranulomatosis.

“Gaucher Disease” as it relates to the present technology means a disease caused by a defect in the glucosylceramidase enzyme (also known as glucocerebrosidase and acid β-glucosidase); a 55.6 KD, 497 amino acids long protein. Glucosylceramidase is responsible for the conversion of glycosylceramide (or glucocerebroside) to ceramide; the defect thus leads to an accumulation of glycosylceramide. Its activity is stimulated by BMP and is dependent on saposins. Gaucher's disease is the most common of the lysosomal storage diseases. Fatty material can collect in the spleen, liver, kidneys, lungs, brain and bone marrow. Symptoms may include enlarged spleen and liver, liver malfunction, skeletal disorders and bone lesions that may be painful, severe neurologic complications, swelling of lymph nodes and (occasionally) adjacent joints, distended abdomen, a brownish tint to the skin, anemia, low blood platelets and yellow fatty deposits on the sclera. Persons affected most seriously may also be more susceptible to infection. The disease shows autosomal recessive inheritance and therefore affects both males and females. Different mutations of glucosylceramidase determine the remaining activity of the enzyme, and, to a large extent, the phenotype. Research suggests that heterozygotes for particular glucosylceramidase mutations are at an increased risk of Parkinson's disease and particular malignancies (non-Hodgkin lymphoma, melanoma and pancreatic cancer). Glycosylceramide is a cell membrane constituent of red and white blood cells. The macrophages that clear these cells are unable to eliminate the waste product, which accumulates in fibrils, and turn into Gaucher cells, which appear on light microscopy to resemble crumpled-up paper.

Gaucher's disease has three common clinical subtypes. Each type has been linked to particular mutations. In all, there are about 80 known mutations. Type I (or nonneuropathic type) Gaucher Disease is the most common form of the disease occurring in approximately 1 in 50,000 live births. It occurs most often among persons of Ashkenazi Jewish heritage, 100 times the occurrence in the general populace. Symptoms may begin early in life or in adulthood and include enlarged liver and grossly enlarged spleen (together hepatosplenomegaly); the spleen can rupture and cause additional complications. Skeletal weakness and bone disease may be extensive. Spleen enlargement and bone marrow replacement cause anemia, thrombocytopenia and leukopenia. The brain is not affected, but there may be lung and, rarely, kidney impairment. Patients in this group usually bruise easily (due to low levels of platelets) and experience fatigue due to low numbers of red blood cells. Depending on disease onset and severity, type 1 patients may live well into adulthood. Many patients have a mild form of the disease or may not show any symptoms. Type II (or acute infantile neuropathic Gaucher's disease) typically begins within 6 months of birth and has an incidence rate of approximately 1 in 100,000 live births. Symptoms include an enlarged liver and spleen, extensive and progressive brain damage, eye movement disorders, spasticity, seizures, limb rigidity, and a poor ability to suck and swallow. Affected children usually die by age 2. Type III (the chronic neuropathic Gaucher Disease) can begin at any time in childhood or even in adulthood and occurs in approximately 1 in 100,000 live births. It is characterized by slowly progressive but milder neurologic symptoms compared to the acute or type 2 version. Major symptoms include an enlarged spleen and/or liver, seizures, poor coordination, skeletal irregularities, eye movement disorders, blood disorders including anemia and respiratory problems. Patients often live into their early teen years and adulthood.

The National Gaucher Foundation states that around 1 in 100 people in the general U.S. population is a carrier for type 1 Gaucher's disease, giving a prevalence of 1 in 40,000; among Ashkenazi Jews the rate of carriers is considerably higher, at roughly 1 in 15. Type 2 Gaucher's disease shows no particular preference for any ethnic group. Type 3 Gaucher's disease is especially common in the population of the Northern Swedish region of Norrbotten where the incidence of the disease is 1 in 50,000. For type 1 and most type 3 patients, enzyme replacement treatment with intravenous recombinant glucosylceramidase can decrease liver and spleen size, reduce skeletal abnormalities, and reverse other manifestations. The rarity of the disease means that dose-finding studies have been difficult to conduct, so there remains controversy over the optimal dose and dosing frequency. Due to the low incidence, this has become an orphan drug in many countries. Successful bone marrow transplantation cures the non-neurological manifestations of the disease, because it introduces a monocyte population with active glucosylceramidase. However, this procedure carries significant risk and is rarely performed in Gaucher patients. Surgery to remove the spleen (splenectomy) may be required on rare occasions if the patient is anemic or when the enlarged organ affects the patient's comfort. Blood transfusion may benefit some anemic patients. Other patients may require joint replacement surgery to improve mobility and quality of life. Other treatment options include antibiotics for infections, antiepileptics for seizures, bisphosphonates for bone lesions, and liver transplants. Substrate reduction therapy may prove to be effective in stopping Type 2, as it can cross through the blood barrier into the brain. There is currently no effective treatment for the severe brain damage that may occur in patients with types 2 and 3 Gaucher disease.

In a further embodiment, the present disclosure provides a method of treating a neurodegenerative or metabolic disease or disorder, the method comprising administering to the patient in need thereof a composition comprising a therapeutically effective amount of a compound of Formula I to Formula VII, wherein the mucopolysaccharidosis is selected from MPS I, Hurler syndrome, Hurler-Scheie syndrome, Scheie syndrome, MPS II, Hunter syndrome), MPS III, Sanfilippo syndrome type A, Sanfilippo syndrome type B, Sanfilippo syndrome type C, Sanfilippo syndrome type D, MPS IV, Morquio syndrome type A, Morquio syndrome type B), MPS VI, Maroteaux-Lamy syndrome, MPS VII, Sly syndrome, MPS IX, and Natowicz syndrome.

In a further embodiment, the present disclosure provides a method of treating a neurodegenerative or metabolic disease or disorder, the method comprising administering to the patient in need thereof a composition comprising a therapeutically effective amount of a compound of Formula I to Formula VII, wherein the lysosomal glycogen storage disorder is selected from Pompe disease and glycogen storage disease type II.

In a further embodiment, the present disclosure provides a method of treating a neurodegenerative or metabolic disease or disorder, the method comprising administering to the patient in need thereof a composition comprising a therapeutically effective amount of a compound of Formula I to Formula VII, wherein the oligosaccharidosis is selected from Schindler disease, Sialidosis Type I, Sialidosis type II, aspartylglucosaminuria, alpha-mannosidosis, beta-mannosidosis, and fucosidosis.

“Sialidosis” or “Mucolipidosis type 1” as it relates to the present technology means a caused by a defect in the sialidase enzyme (or alpha-neuraminidase). Sialidase is responsible for the conversion of GM3 to lactosylceramide; the defect thus leads to an accumulation of GM3. Its activity is stimulated by BMP and is dependent on saposins. Sialidosis is inherited in an autosomal recessive manner. Symptoms are either present at birth or develop within the first year of life. In many affected infants, excessive swelling throughout the body is noted at birth. These infants are often born with coarse facial features, such as a flat nasal bridge, puffy eyelids, enlargement of the gums, and excessive tongue size (macroglossia). Many infants with are also born with skeletal malformations such as hip dislocation. Infants often develop sudden involuntary muscle contractions (called myoclonus) and have red spots in their eyes (cherry red macules). They are often unable to coordinate voluntary movement (called ataxia). Tremors, impaired vision, and seizures also occur. Tests reveal abnormal enlargement of the liver (hepatomegaly) and spleen (splenomegaly) and extreme abdominal swelling. Infants generally lack muscle tone (hypotonia) and have mental retardation that is either initially or progressively severe. Many patients suffer from failure to thrive and from recurrent respiratory infections. Most infants with ML I die before the age of 1 year. Sialidosis may be sub-categorized according to the age at which symptoms begin and the types of symptoms present. The effects of the disease may range from mild to severe.

Sialidosis is a rare disorder that has no racial predilection. Very little population data are available, but a study from the Netherlands reported a frequency of approximately 1 case in 2,175,000 live births. However, this rate may not apply to all populations, some of which could have a higher incidence; moreover, missed clinical recognition is an important factor when newborn screening is not an option.

“Metachromatic Leukodystrophy (MLD)” or “Arylsulfatase A deficiency” as it relates to the present technology means a disease caused by a defect in the arylsulfatase A enzyme (or cerebroside-sulfatase). Arylsulfatase A is responsible for the conversion of sulfatide (or cerebroside 3-sulfate) to galactosylceramide; the defect thus leads to an accumulation of sulfatide. Its activity is stimulated by BMP and is dependent on saposins. It is a lysosomal storage disease which is commonly listed in the family of leukodystrophies. Leukodystrophies affect the growth and/or development of myelin, the fatty covering which acts as an insulator around nerve fibers throughout the central and peripheral nervous systems. Like many other genetic disorders that affect lipid metabolism, there are several forms of MLD, which are late infantile, juvenile, and adult: In the late infantile form, which is the most common form MLD, affected children begin having difficulty walking after the first year of life. Symptoms include muscle wasting and weakness, muscle rigidity, developmental delays, progressive loss of vision leading to blindness, convulsions, impaired swallowing, paralysis, and dementia. Children may become comatose. Untreated, most children with this foil of MLD die by age 5, often much sooner. Children with the juvenile loan of MLD (onset between 3-10 years of age) usually begin with impaired school performance, mental deterioration, and dementia and then develop symptoms similar to the late infantile form but with slower progression. Age of death is variable, but normally within 10 to 15 years of symptom onset. The adult form commonly begins after age 16 as a psychiatric disorder or progressive dementia. Adult-onset MLD progresses more slowly than the late infantile and juvenile forms, with a protracted course of a decade or more. In rare cases the body can compensate for the deficiency and the person will exhibit no symptoms.

In a further embodiment, the present disclosure provides a method of treating a neurodegenerative or metabolic disease or disorder, the method comprising administering to the patient in need thereof a composition comprising a therapeutically effective amount of a compound of Formula I to Formula VII, wherein the mucolipidosis is selected from mucolipidosis II, I-cell disease, mucolipidosis III, pseudo-Hurler polydystrophy, and mucolipidosis IV.

In a further embodiment, the present disclosure provides a method of treating a neurodegenerative or metabolic disease or disorder, the method comprising administering to the patient in need thereof a composition comprising a therapeutically effective amount of a compound of Formula I to Formula VII, wherein the lipid storage disorder is selected from Wolman disease, cholesteryl ester storage disease, and Farber disease.

In a further embodiment, the present disclosure provides a method of treating a neurodegenerative or metabolic disease or disorder, the method comprising administering to the patient in need thereof a composition comprising a therapeutically effective amount of a compound of Formula I to Formula VII, wherein the glycoproteinosis is selected from alpha-mannosidosis, beta-mannosidosis, fucosidosis, aspartylglucosaminuria.

In a further embodiment, the present disclosure provides a method of treating a neurodegenerative or metabolic disease or disorder, the method comprising administering to the patient in need thereof a composition comprising a therapeutically effective amount of a compound of Formula I to Formula VII, wherein the neuronal ceroid lipofuscinosis is selected from infantile NCL, CLN1, CLN5, late infantile NCL, CLN2, juvenile NCL, CLN3, adult NCL, Kufs disease, CLN4, congenital NCL, and CLN10.

In a further embodiment, the present disclosure provides a method of treating a neurodegenerative or metabolic disease or disorder, the method comprising administering to the patient in need thereof a composition comprising a therapeutically effective amount of a compound of Formula I to Formula VII, wherein the peroxisomal biogenesis disorder is selected from Zellweger syndrome, neonatal adrenoleukodystrophy, and infantile Refsum disease.

In a further embodiment, the present disclosure provides a method of treating a neurodegenerative or metabolic disease or disorder, the method comprising administering to the patient in need thereof a composition comprising a therapeutically effective amount of a compound of Formula I to Formula VII, wherein the other lysosomal storage disorder is selected from Danon disease, cystinosis, sialic acid storage disease, Salla disease, lysosomal acid lipase deficiency (LAL-D), multiple sulfatase deficiency, pycnodysostosis, galactosialidosis, hyaluronidase deficiency (MPS IX), Hermansky-Pudlak syndrome, and pseudoxanthoma elasticum (PXE).

In a further embodiment, the present disclosure provides a method of treating a neurodegenerative or metabolic disease or disorder, the method comprising administering to the patient in need thereof a composition comprising a therapeutically effective amount of a compound of Formula I to Formula VII, wherein the neurodegenerative or metabolic disorder is selected from Niemann Pick disease Type C 1 or Niemann Pick disease Type C 2.

“Nieman-Pick Disease” or “NP” as it relates to the present technology means a group of rare genetic diseases of varying severity. These are inherited metabolic disorders in which sphingomyelin accumulates in lysosomes in cells of many organs. NP types A, A/B, and B are caused by mutations in the SMPD1 gene, which causes a deficiency of an acid sphingomyelinase (ASM). NP type C is now considered a separate disease, as SMPD1 is not involved, and there is no deficiency in ASM. State. Arimoclomol targets NPC etiology by both NPC1-independent and NPC1-dependent pathways: (1) via the NPC1-independent pathway, arimoclomol upregulates expression of all CLEAR genes, thereby mitigating the deleterious effects of impaired cholesterol trafficking and improving overall cell health; (2) via the NPC1-dependent pathway, CLEAR gene upregulation increases production of the NPC1 protein. Though still mutated, overproducing the reduced function protein improves lysosomal function and cholesterol elimination.

“Nieman-Pick Disease Type C” or “NPC” as it relates to the present technology means a rare progressive genetic disorder characterized by an inability of the body to transport cholesterol and other fatty substances (lipids) inside of cells. This leads to the abnormal accumulation of these substances within various tissues of the body, including brain tissue.

“Nieman-Pick Type C Protein 1” or “NPC1” as it relates to the present technology means the gene encoding the Niemann-Pick type C protein 1, also referred to in the art as the NPC intracellular cholesterol transporter 1. The term “NPC1” refers to the protein product of the NPC1 gene. In patients with NPC, the NPC1 protein has substantially diminished quantity and functionality because mutations in NPC genes prevent most NPC1 protein from completing assembly and maturation in the endoplasmic reticulum and Golgi apparatus. Furthermore, the small amount of NPC1 protein that does localize in the lysosome is thought to be less effective, leading to cholesterol accumulation and neuronal death.

“NPC1^{-/-}” as it relates to the present technology means a mouse model that possesses a null allele mutation in the NPC1 gene which results in expression of a non-functional mutant NPC1 protein.

“NPC1^{nmf164}” as it relates to the present technology means a mouse model that possesses a point mutation in the NPC1 genes which results in expression of a mutant NPC1 protein with reduced function compared to wild-type NPC1 protein.

“NPC1-dependent” as it relates to the present technology means a biological pathway that involves the NPC1 gene and/or the NPC1 protein.

“NPC1-independent” as it relates to the present technology means a biological pathway that does not involve the NPC1 gene or NPC1 protein.

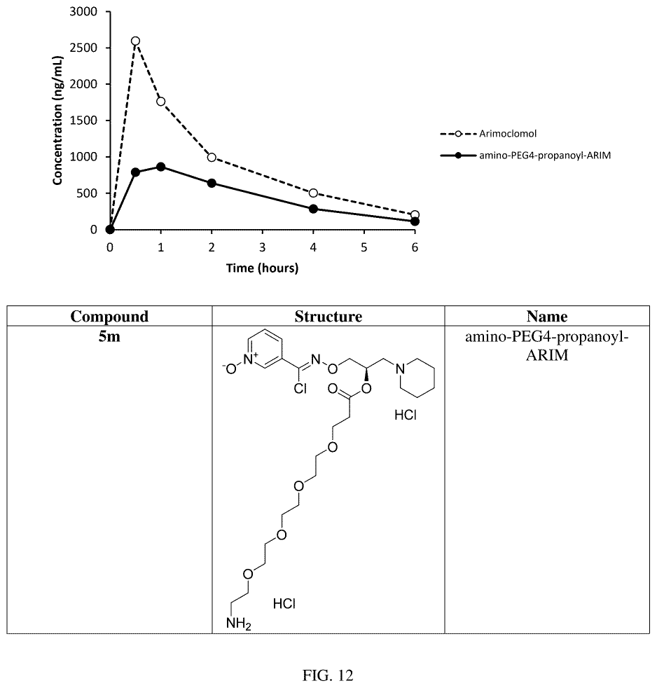

In certain patients with NPC, the NPC1 protein has substantially diminished quantity and functionality because mutations in NPC genes prevent most NPC1 protein from completing assembly and maturation in the endoplasmic reticulum and Golgi apparatus (FIG. 12, Panel B). Furthermore, the small amount of NPC1 protein that does localize in the lysosome is thought to be less effective, leading to cholesterol accumulation and neuronal death.

“Fabry Disease” as it relates to the present technology means a disease is caused by a defect in the a-galactosidase A enzyme. a-galactosidase A is responsible for the conversion of globotriaosylceramide to lactosylceramide; the defect thus leads to an accumulation of globotriaosylceramide (also abbreviated as Gb3, GL-3, or ceramide trihexoside). Its activity is stimulated by BMP and is dependent on saposins. Fabry disease is also known as Anderson-Fabry disease, Angiokeratoma corporis diffusum, Ruiter-Pompen-Wyers syndrome, Ceramide trihexosidosis, and Sweeley-Klionsky disease. It is an X-linked recessive (inherited) disease that affects hemizygous males, as well as both heterozygous and homozygous females; males tend to experience the most severe clinical symptoms, while females vary from virtually no symptoms to those as serious as males. This variability is thought to be due to X-inactivation patterns during embryonic development of the female. Symptoms include anhidrosis (lack of sweating), fatigue, angiokeratomas (benign cutaneous injury of capillaries), burning extremity pain and ocular involvement. Angiokeratomas are tiny, painless papules that appear at any region of the body, but are predominant on the thighs, buttocks, lower abdomen, and groin. Cosmetic ocular involvement may be present showing cornea verticillata (also known as vortex keratopathy). Keratopathy may be the presenting feature in asymptomatic carriers and must be differentiated from other causes of vortex keratopathy (e.g. drug deposition in the cornea). Other ocular findings that can be seen include conjunctival aneurysms, posterior spoke-like cataracts, papilloedema, macular edema, optic atrophy and retinal vascular dilation. Kidney complications are a common and serious effect of the disease; renal insufficiency and renal failure may worsen throughout life. Proteinuria is often the first sign of kidney involvement. Cardiac complications may also occur; heart related effects worsen with age and may lead to increased risk of heart disease. Cerebrovascular effects lead to an increased risk of stroke. Other symptoms include tinnitus, vertigo, nausea, and diarrhea. Symptoms are typically first experienced in early childhood and can be very difficult to understand; the rarity of Fabry disease to many clinicians sometimes leads to misdiagnoses or ignorance. Manifestations of the disease usually increase in number and severity as an individual age.

In a further embodiment, the present disclosure provides a method of treating a neurodegenerative or metabolic disease or disorder, the method comprising administering to the patient in need thereof a composition comprising a therapeutically effective amount of a compound of Formula I to Formula VII, wherein the lysosomal storage disorder is selected from sphingolipidoses, mucopolysaccharidoses (MPS), lysosomal glycogen storage disorders, oligosaccharidoses, mucolipidoses, lipid storage disorders, glycoproteinoses, neuronal ceroid lipofuscinoses (NCL), peroxisomal biogenesis disorders (Zellweger spectrum disorders), and other lysosomal storage disorders.

In a further embodiment, the present disclosure provides a method of treating a neurodegenerative or metabolic disease or disorder, the method comprising administering to the patient in need thereof a composition comprising a therapeutically effective amount of a compound of Formula I to Formula VII, wherein the sphingolipidosis is selected from Gaucher disease type 1, Gaucher disease type 2, Gaucher disease type 3, Fabry disease, Niemann-pick disease type A, Niemann-pick disease type B, Niemann-pick disease type C, Tay-Sachs disease, Sandhoff disease, GM1 gangliosidosis, metachromatic leukodystrophy (MLD), Krabbe disease, and Farber lipogranulomatosis.

“Krabbe Disease” as it relates to the present technology means a disease caused by a defect in the β-galactosylceramidase enzyme. β-galactosylceramidase is responsible for the conversion of galactosylceramide to ceramide; the defect thus leads to an accumulation of galactosylceramide. Its activity is stimulated by BMP and is dependent on saposins.

Krabbe disease is also known as globoid cell leukodystrophy or galactosylceramide lipidosis. It is a rare, often fatal degenerative autosomal recessive disorder that affects the myelin sheath of the nervous system. It occurs in about 1 in 100,000 births. A higher prevalence, about 1 in 6,000 has been reported in some Arab communities in Israel. Krabbe disease is caused by mutations in the GALC gene, which causes a deficiency of the galactosylceramidase enzyme. The lipid buildup affects the growth of the nerve's protective myelin sheath (the covering that insulates many nerves) and causes severe degeneration of motor skills. Infants with Krabbe disease are normal at birth. Symptoms begin between the ages of 3 and 6 months with irritability, fevers, limb stiffness, seizures, feeding difficulties, vomiting, and slowing of mental and motor development. In the first stages of the disease, doctors often mistake the symptoms for those of cerebral palsy. Other symptoms include muscle weakness, spasticity, deafness, optic atrophy and blindness, paralysis, and difficulty when swallowing. Prolonged weight loss may also occur. There are also juvenile- and adult-onset cases of Krabbe disease, which have similar symptoms but slower progression. In infants, the disease is generally fatal before age 2. Patients with late-onset Krabbe disease tend to have a slower progression of the disease and live significantly longer.

“Farber Disease” as it relates to the present technology means a disease associated with a defect is the acid ceramidase enzyme, which is responsible for the conversion of ceramide to sphingosine (and fatty acid); the defect thus leads to an accumulation of ceramide. Its activity is stimulated by BMP and is dependent on saponins. Acid ceramidase is also known as N-acylsphingosine amidohydrolase and is coded by the gene ASAH1. It is a heterodimeric protein consisting of a nonglycosylated alpha subunit and a glycosylated beta subunit that is cleaved to the mature enzyme post-translationally. Farber disease is also known as Farber's lipogranulomatosis, ceramidase deficiency, Fibrocytic dysmucopolysaccharidosis, and Lipogranulomatosis. It is an extremely rare autosomal recessive disease characterized by abnormalities in the joints, liver, throat, tissues and central nervous system. The liver, heart, and kidneys may also be affected. Symptoms are typically seen in the first few weeks of life and include impaired motor and mental ability and difficulty with swallowing. Other symptoms may include arthritis, swollen lymph nodes and joints, hoarseness, nodules under the skin (and sometimes in the lungs and other parts of the body), chronic shortening of muscles or tendons around joints, and vomiting. Affected persons may require the insertion of a breathing tube. In severe cases, the liver and spleen are enlarged. Currently there is no specific treatment for Farber disease. Corticosteroids can help relieve pain. Nodes can be treated with bone marrow transplants, in certain instances, or may be surgically reduced or removed. Most children with the classic form of Farber's disease die by age 2, usually from lung disease. Individuals having a milder form of the disease may live into their teenage years.

“Tay-Sachs Disease” as it relates to the present disclosure, means a disease is caused by a genetic mutation in the HEXA gene on chromosome 15, which codes a subunit of the hexosaminidase enzyme known as hexosaminidase A. It is inherited in an autosomal recessive manner. The mutation disrupts the activity of the enzyme, which results in the build-up of the molecule GM2 ganglioside within cells, leading to toxicity. Diagnosis may be supported by measuring the blood hexosaminidase A level or genetic testing. Tay-Sachs disease is a type of GM2 gangliosidosis and sphingolipidosis. Tay-Sachs disease is caused by mutations in the HEXA gene on chromosome 15, which encodes the alpha-subunit of beta-N-acetylhexosaminidase A, a lysosomal enzyme. Hexosaminidase A is a vital hydrolytic enzyme, found in the lysosomes, that breaks down sphingolipids. When hexosaminidase A is no longer functioning properly, the lipids accumulate in the brain and interfere with normal biological processes. Hexosaminidase A specifically breaks down fatty acid derivatives called gangliosides; these are made and biodegraded rapidly in early life as the brain develops. Patients with and carriers of Tay-Sachs can be identified by a simple blood test that measures hexosaminidase A activity. In a further embodiment, the present technology describes a method of treating a neurodegenerative or metabolic disease or disorder, the method comprising administering to the patient in need thereof a therapeutically effective amount of the compound of Formula I, wherein the mucopolysaccharidosis is selected from MPS I, Hurler syndrome, Hurler-Scheie syndrome, Scheie syndrome, MPS II, Hunter syndrome), MPS III, Sanfilippo syndrome type A, Sanfilippo syndrome type B, Sanfilippo syndrome type C, Sanfilippo syndrome type D, MPS IV, Morquio syndrome type A, Morquio syndrome type B), MPS VI, Maroteaux-Lamy syndrome, MPS VII, Sly syndrome, MPS IX, and Natowicz syndrome.

In a further embodiment, the present disclosure provides a method of treating a neurodegenerative or metabolic disease or disorder, the method comprising administering to the patient in need thereof a composition comprising a therapeutically effective amount of a compound of Formula I to Formula VII, wherein the lysosomal glycogen storage disorder is selected from Pompe disease and glycogen storage disease type II.

In a further embodiment, the present disclosure provides a method of treating a neurodegenerative or metabolic disease or disorder, the method comprising administering to the patient in need thereof a composition comprising a therapeutically effective amount of a compound of Formula I to Formula VII, wherein the oligosaccharidosis is selected from Schindler disease, Sialidosis Type I, Sialidosis type II, aspartylglucosaminuria, alpha-mannosidosis, beta-mannosidosis, and fucosidosis.

In a further embodiment, the present disclosure provides a method of treating a neurodegenerative or metabolic disease or disorder, the method comprising administering to the patient in need thereof a composition comprising a therapeutically effective amount of a compound of Formula I to Formula VII, wherein the mucolipidosis is selected from mucolipidosis II, I-cell disease, mucolipidosis III, pseudo-Hurler polydystrophy, and mucolipidosis IV.

In a further embodiment, the present disclosure provides a method of treating a neurodegenerative or metabolic disease or disorder, the method comprising administering to the patient in need thereof a composition comprising a therapeutically effective amount of a compound of Formula I to Formula VII, wherein the lipid storage disorder is selected from Wolman disease, cholesteryl ester storage disease, and Farber disease.

In a further embodiment, the present disclosure provides a method of treating a neurodegenerative or metabolic disease or disorder, the method comprising administering to the patient in need thereof a composition comprising a therapeutically effective amount of a compound of Formula I to Formula VII, wherein the glycoproteinosis is selected from alpha-mannosidosis, beta-mannosidosis, fucosidosis, aspartylglucosaminuria.

In a further embodiment, the present disclosure provides a method of treating a neurodegenerative or metabolic disease or disorder, the method comprising administering to the patient in need thereof a composition comprising a therapeutically effective amount of a compound of Formula I to Formula VII, wherein the neuronal ceroid lipofuscinosis is selected from infantile NCL, CLN1, CLN5, late infantile NCL, CLN2, juvenile NCL, CLN3, adult NCL, Kufs disease, CLN4, congenital NCL, and CLN10.

In a further embodiment, the present disclosure provides a method of treating a neurodegenerative or metabolic disease or disorder, the method comprising administering to the patient in need thereof a composition comprising a therapeutically effective amount of a compound of Formula I to Formula VII, wherein the peroxisomal biogenesis disorder is selected from Zellweger syndrome, neonatal adrenoleukodystrophy, and infantile Refsum disease.

In a further embodiment, the present disclosure provides a method of treating a neurodegenerative or metabolic disease or disorder, the method comprising administering to the patient in need thereof a composition comprising a therapeutically effective amount of a compound of Formula I to Formula VII, wherein the other lysosomal storage disorder is selected from Danon disease, cystinosis, sialic acid storage disease, Salla disease, lysosomal acid lipase deficiency (LAL-D), multiple sulfatase deficiency, pycnodysostosis, galactosialidosis, hyaluronidase deficiency (MPS IX), Hermansky-Pudlak syndrome, and pseudoxanthoma elasticum (PXE).

In a further embodiment, the present disclosure provides a method of treating a neurodegenerative or metabolic disease or disorder, the method comprising administering to the patient in need thereof a composition comprising a therapeutically effective amount of a compound of Formula I to Formula VII, wherein the Niemann Pick disease Type C is Niemann Pick disease Type C 1 or Niemann Pick disease Type C 2.

In a further embodiment, the present disclosure provides a method of treating a neurodegenerative or metabolic disease or disorder, the method comprising administering to the patient in need thereof a composition comprising a therapeutically effective amount of a compound of Formula I to Formula VII, wherein the therapeutically effective amount is between 1 mg and 5000 mg per day. In a still further embodiment, the therapeutically effective amount is between 500 mg and 2000 mg, alternatively from about 50 mg to about 2500 mg, alternatively about 100 mg to about 2000 mg, alternatively about 200 mg to about 1500 mg, alternatively about 500 mg to about 1000 mg per day.

In a further embodiment, the present disclosure provides a method of treating a neurodegenerative or metabolic disease or disorder, the method comprising administering to the patient in need thereof a composition comprising a therapeutically effective amount of a compound of Formula I to Formula VII, wherein the therapeutically effective amount is administered in 1 to 3 unit doses.

In a further embodiment, the present disclosure provides a method of treating a neurodegenerative or metabolic disease or disorder, the method comprising administering to the patient in need thereof a composition comprising a therapeutically effective amount of a compound of Formula I to Formula VII, wherein the therapeutically effective amount is selected from an equivalent of 47 mg t.i.d. of arimoclomol citrate, and equivalent of 62 mg t.i.d. arimoclomol citrate, an equivalent of 93 mg t.i.d. of arimoclomol citrate. and an equivalent of 124 mg t.i.d. of arimoclomol citrate. Table 8 describes how the aforementioned equivalents are selected based on patient weight.

TABLE 8
Recommended dosing schedule for patients 2 years and older according
to MIPLYFFA® insert.

| | Recommended Equivalent |
|---|---|
| Patient Body Weight | of Arimoclomol base |
| 8 kg to 22 kg | 47 mg t.i.d. |
| >22-38 kg | 62 mg t.i.d. |
| >38-55 kg | 93 mg t.i.d. |
| >55 kg | 124 mg t.i.d. |

In a further embodiment, the present disclosure provides a method of treating a neurodegenerative or metabolic disease or disorder, the method comprising administering to the patient in need thereof a composition comprising a therapeutically effective amount of a compound of Formula I to Formula VII, wherein the therapeutically effective amount is administered in 1 unit dose.

In a further embodiment, the present disclosure provides a method of treating a neurodegenerative or metabolic disease or disorder, the method comprising administering to the patient in need thereof a composition comprising a therapeutically effective amount of a compound of Formula I to Formula VII, wherein the therapeutically effective amount is between 1 mg and 5000 mg per day. In a further embodiment, he therapeutically effective amount is between 500 mg and 2000 mg, alternatively from about 50 mg to about 2500 mg, alternatively about 100 mg to about 2000 mg, alternatively about 200 mg to about 1500 mg, alternatively about 500 mg to about 1000 mg per day.

In a further embodiment, the present disclosure provides a method of treating a neurodegenerative or metabolic disease or disorder, the method comprising administering to the patient in need thereof a composition comprising a therapeutically effective amount of a compound of Formula I to Formula VII, wherein the therapeutically effective amount is administered in 1 to 2 unit doses.

In a further embodiment, the present disclosure provides a method of treating a neurodegenerative or metabolic disease or disorder, the method comprising administering to the patient in need thereof a composition comprising a therapeutically effective amount of a compound of Formula I to Formula VII, wherein the therapeutically effective amount is administered in 1 unit dose.

In a further embodiment, the present disclosure provides a method of treating a neurodegenerative or metabolic disease or disorder, the method comprising administering to the patient in need thereof a composition comprising a therapeutically effective amount of a compound of Formula I to Formula VII, wherein the method further comprises administering a therapeutically effective amount of at least one additional active pharmaceutical ingredient selected from bimoclomol, miglustat, eligustat, N-acetyl-L-leucine, arimoclomol, and 2-hydroxypropyl-p-cyclodextrin, and combinations thereof. In a still further embodiment, the at least one additional active pharmaceutical ingredient is selected from N-acetyl-L-leucine, 2-hydroxypropyl-p-cyclodextrin, and combinations thereof.

In an embodiment, the present disclosure provides a method of treating Niemann-Pick Disease, Type C, in a patient in need thereof less than 2 years of age, the method comprising the steps of: administering to the patient in need thereof a composition comprising a therapeutically effective amount of a compound having a structure of Formula I to Formula VII: or a pharmaceutically acceptable salt thereof, wherein the therapeutically effective amount ranges from about 0.1 mg/kg/day to about 10 mg/kg/day of arimoclomol freebase. Dosing based on patient body weight for patients under 2 years of age are listed in Table 9.

TABLE 9
Recommended dosage for patients less than 2 years of age.

| | | Arimoclomol base |
|---|---|---|
| Patient Body Weight | Arimoclomol citrate dose | dose |
| 8-15 kg | 50 mg t.i.d. | 31 mg t.i.d. |
| >15-22 kg | 75 mg t.i.d. | 47 mg t.i.d. |
| >22-38 kg | 100 mg t.i.d. | 62 mg t.i.d. |
| >38-55 kg | 150 mg t.i.d. | 93 mg t.i.d. |
| >55 kg | 200 mg t.i.d. | 124 mg t.i.d. |

In yet another embodiment, the present disclosure provides a method of treating Niemann-Pick Disease, Type C, in a patient in need thereof less than 2 years of age, the method comprising the steps of: administering to the patient in need thereof a composition comprising a therapeutically effective amount of a compound having a structure of Formula I to Formula VII, or a pharmaceutically acceptable salt thereof, wherein the therapeutically effective amount ranges from about 1 mg/kg/day to about 8 mg/kg/day. In a embodiment, the therapeutically effective amount is 6 mg/kg/day of arimoclomol freebase.

In yet another embodiment, the present disclosure provides a method of treating Niemann-Pick Disease, Type C, in a patient in need thereof less than 2 years of age, the method comprising the steps of: administering to the patient in need thereof a composition comprising a therapeutically effective amount of a compound having a structure of Formula I to Formula VII, or a pharmaceutically acceptable salt thereof, wherein the therapeutically effective amount is administered in 1 to 3 unit doses. In an embodiment, the therapeutically effective amount is administered in 3 unit doses. In an alternative embodiment, the unit dosage is 2 mg/kg/day of arimoclomol freebase.

In yet another embodiment, the present disclosure provides a method of treating Niemann-Pick Disease, Type C, in a patient in need thereof less than 2 years of age, the method comprising the steps of: administering to the patient in need thereof a composition comprising a therapeutically effective amount of a compound having a structure of Formula I to Formula VII, or a pharmaceutically acceptable salt thereof, wherein the composition is liquid oral dosage formulation. In an alternative embodiment, the liquid oral dosage formulation comprises about 0.1 mg/mL to about 5 mg/mL of the compound of Formula I to Formula VII, or pharmaceutically acceptable salt thereof. In an embodiment, the liquid oral dosage formulation comprises about 3.1 mg/mL of the compound of Formula I to Formula VII, or pharmaceutically acceptable salt thereof.

In yet another embodiment, the present disclosure provides a method of treating Niemann-Pick Disease, Type C, in a patient in need thereof less than 2 years of age, the method comprising the steps of: administering to the patient in need thereof a composition comprising a therapeutically effective amount of a compound having a structure of Formula I to Formula VII, or a pharmaceutically acceptable salt thereof, wherein the composition is liquid oral dosage formulation wherein the liquid oral dosage formulation made by emptying the contents of a 47 mg of arimoclomol citrate capsule into 15 mL of water. The liquid oral formulation may be administered orally or through a feeding tube. In this embodiment, the amount of the liquid oral formulation administered to the patient in need thereof is measured in 0.2 mL increments and remaining solution is discarded and not saved for later use. Alternatively the contents of a 31 mg arimoclomol citrate capsule may be emptied into 10 mL of water; alternatively, the contents of a 62 mg of arimoclomol citrate capsule may be emptied into 20 mL of water. In certain embodiments, the amount of the liquid oral formulation administered is determined according patient body weight according to Table 10.

TABLE 10
Amount of 3.1 mg/mL arimoclomol citrate liquid formulation
administered based on body weight.

| | Recommended dose (exact dose based on |
|---|---|
| Patient body | weight should be calculated by your |
| weight | doctor) |
| 5 kg | 3.2 mL 3 times a day |
| 6 kg | 3.8 mL 3 times a day |
| 7 kg | 4.4 mL 3 times a day |
| 8 kg | 5.2 mL 3 times a day |
| 9 kg | 5.8 mL 3 times a day |
| 10 kg | 6.4 mL 3 times a day |
| 11 kg | 7.0 mL 3 times a day |
| 12 kg | 7.6 mL 3 times a day |
| 13 kg | 8.4 mL 3 times a day |
| 14 kg | 9.0 mL 3 times a day |
| 15 kg | 9.6 mL 3 times a day |
| 16 kg | 10.2 mL 3 times a day |
| 17 kg | 10.8 mL 3 times a day |

In an embodiment, the present disclosure provides for a kit comprising: a composition comprising a therapeutically effective amount of a prodrug of arimoclomol; and instructions for use. In a further embodiment, the kit further comprises a composition comprising a therapeutically effective amount of at least one additional active pharmaceutical ingredient. In certain embodiments, the at least one additional active pharmaceutical ingredient is selected from arimoclomol, bimoclomol, miglustat, eligustat, and N-acetyl-L-leucine.

### EXAMPLES

The presently described technology and its advantages will be better understood by reference to the following examples. These examples are provided to describe specific embodiments of the present technology. By providing these specific examples, it is not intended limit the scope and spirit of the present technology. It will be understood by those skilled in the art that the full scope of the presently described technology encompasses the subject matter defined by the claims appending this specification, and any alterations, modifications, or equivalents of those claims.

### Example 1—Pharmacokinetic Comparison of Arimoclomol Prodrugs to Arimoclomol

Compounds 14a, 14 h, 7b, and 3c or arimoclomol were orally administered and plasma levels were monitored for 6 hours.

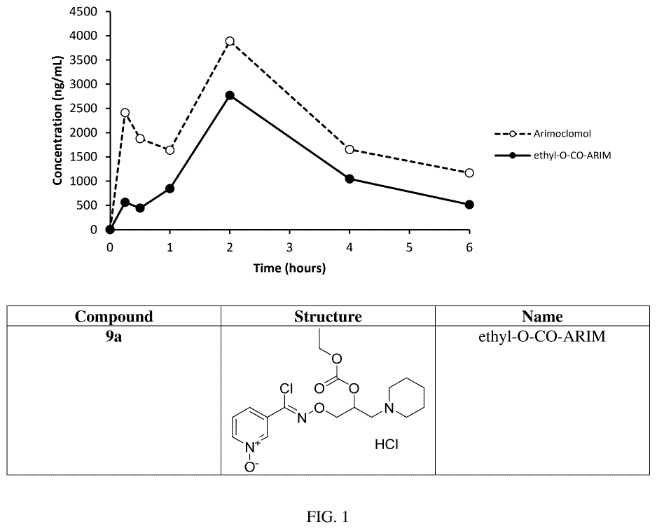

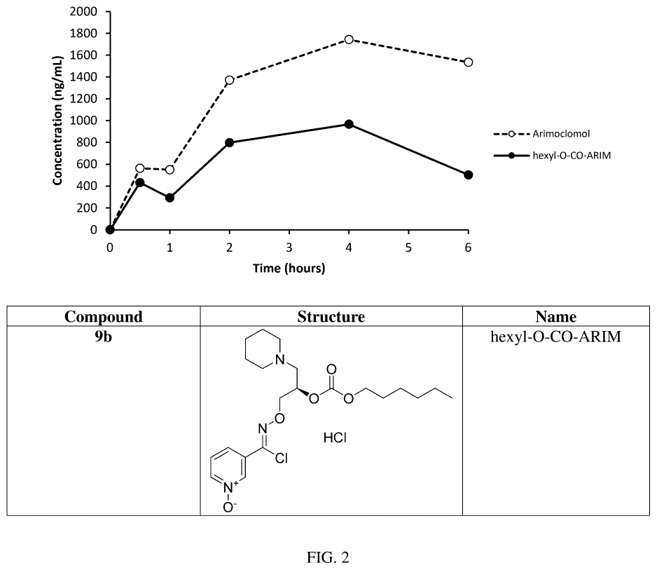

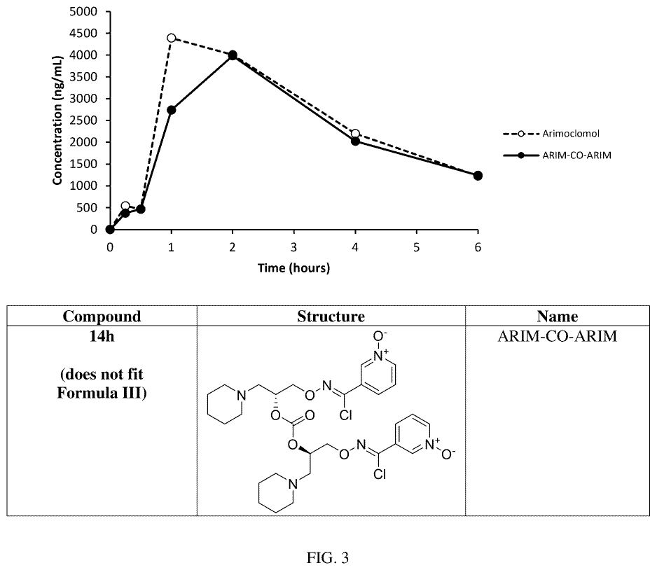

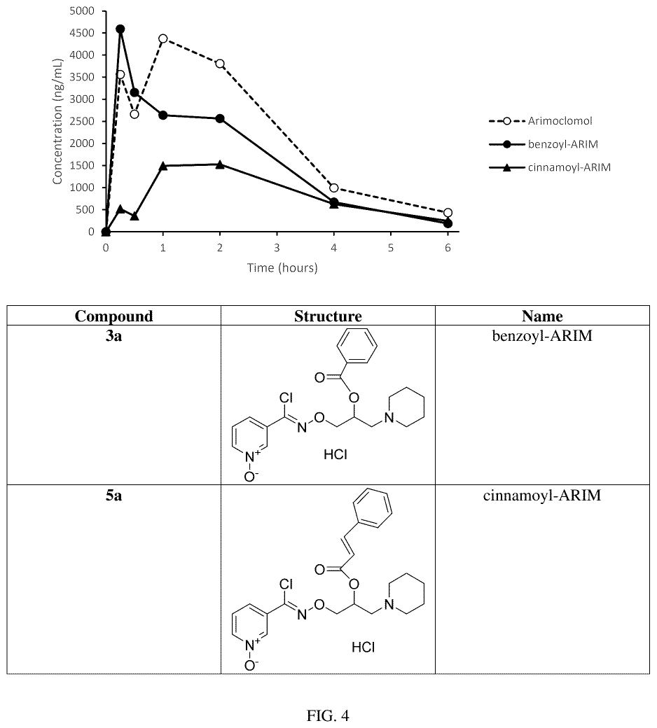

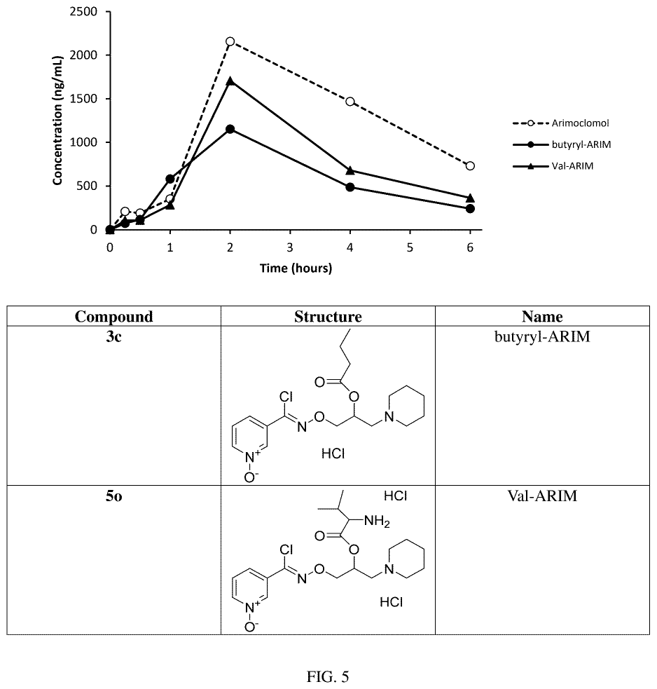

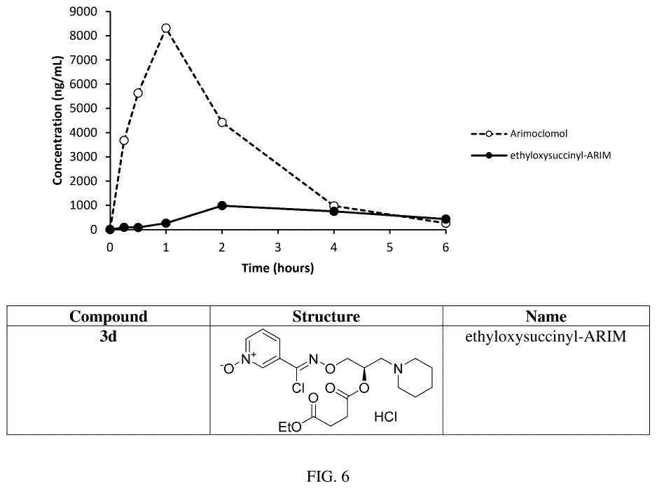

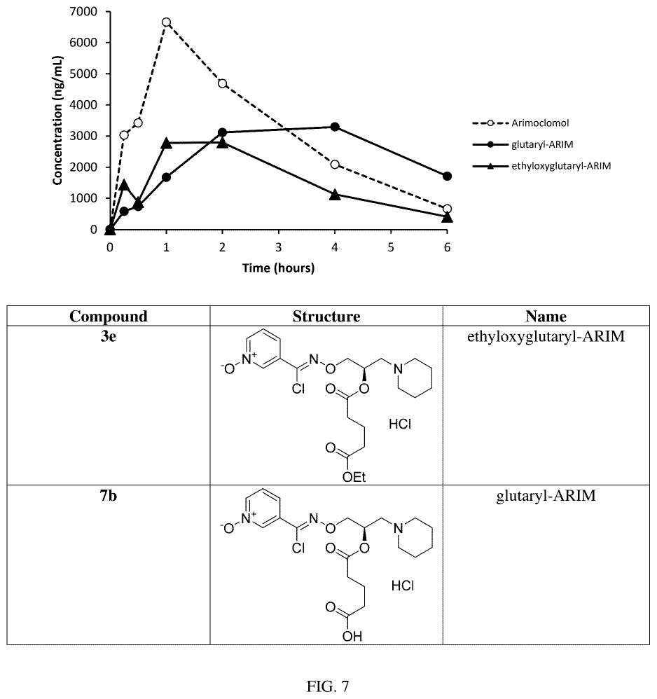

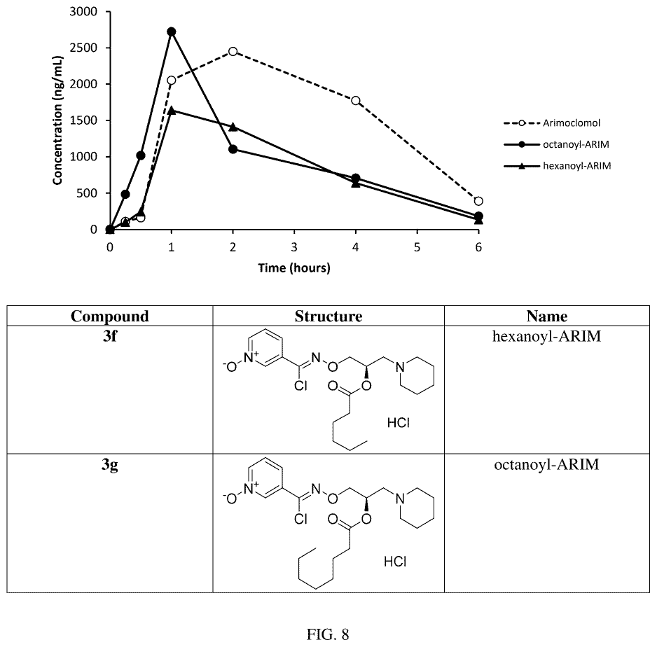

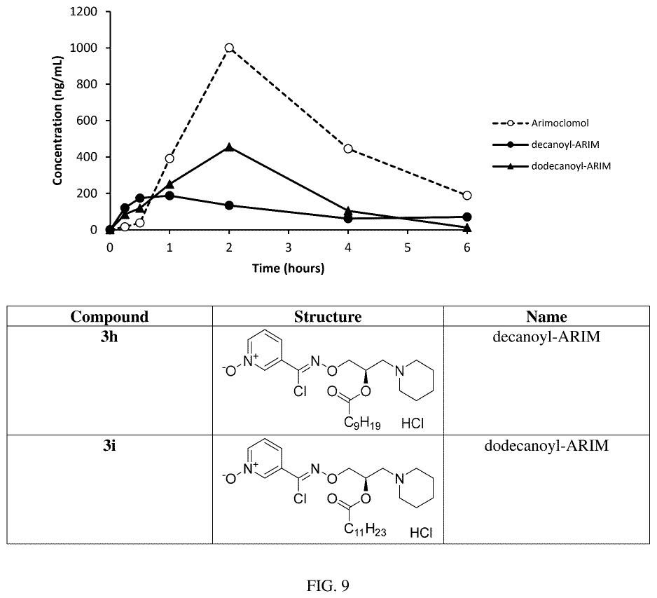

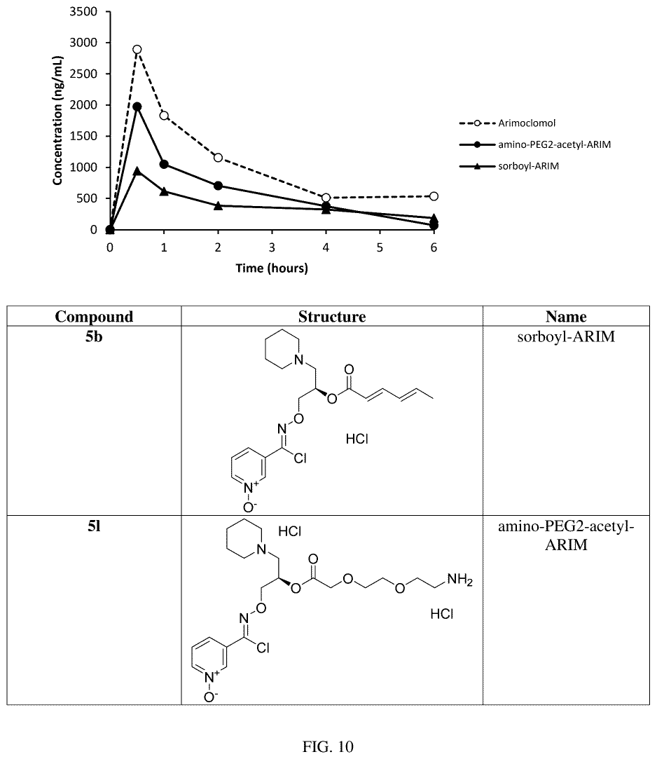

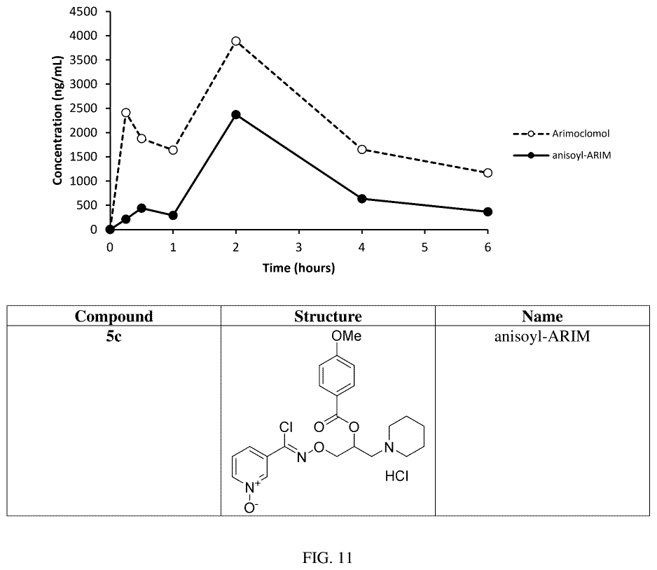

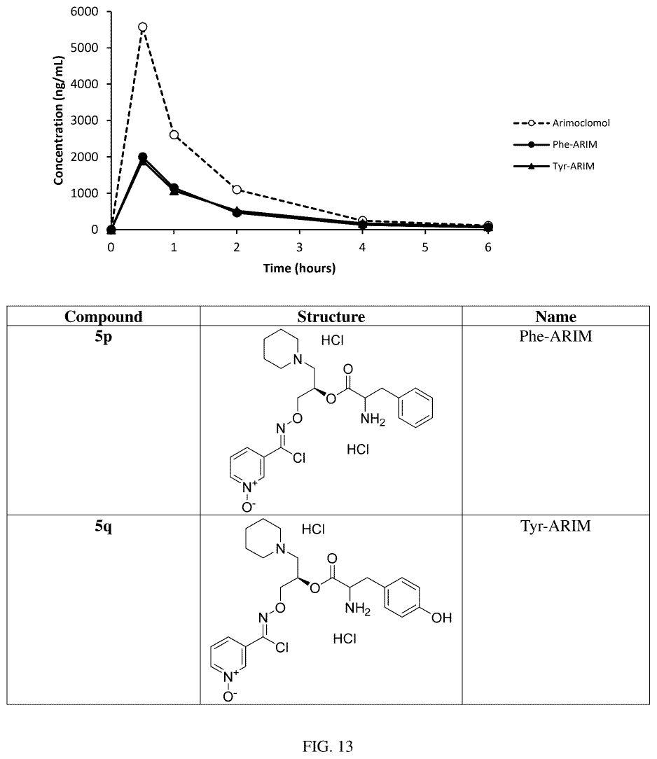

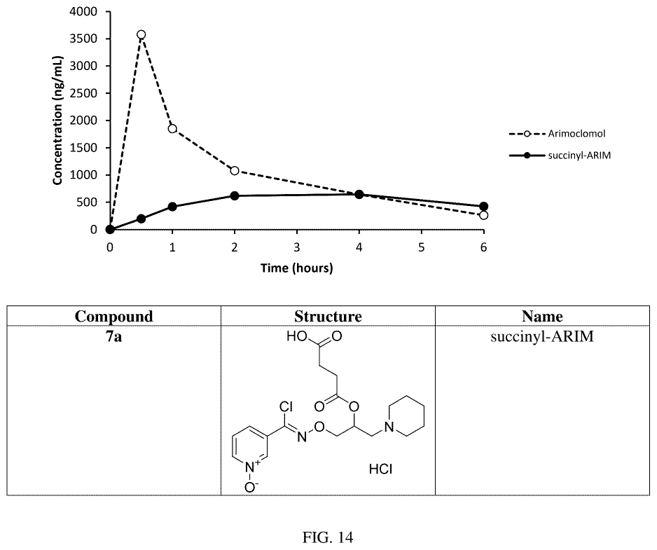

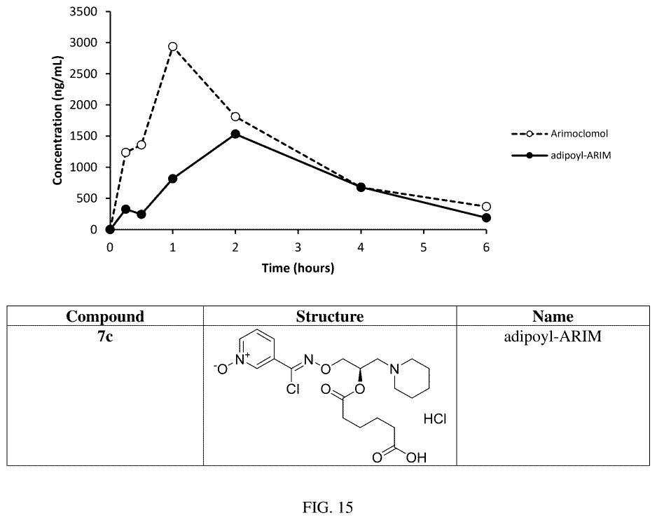

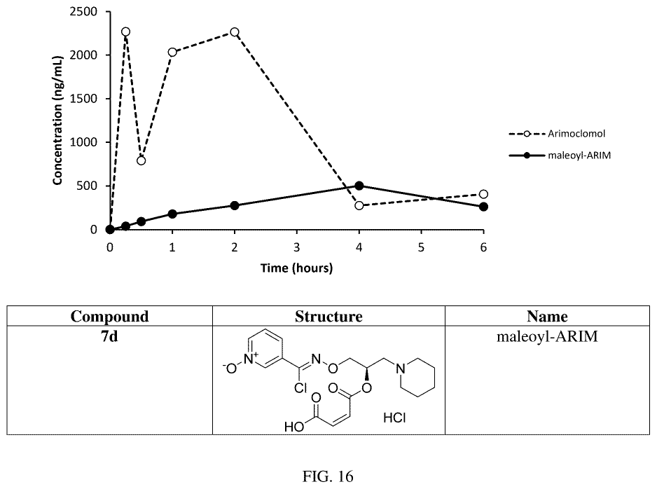

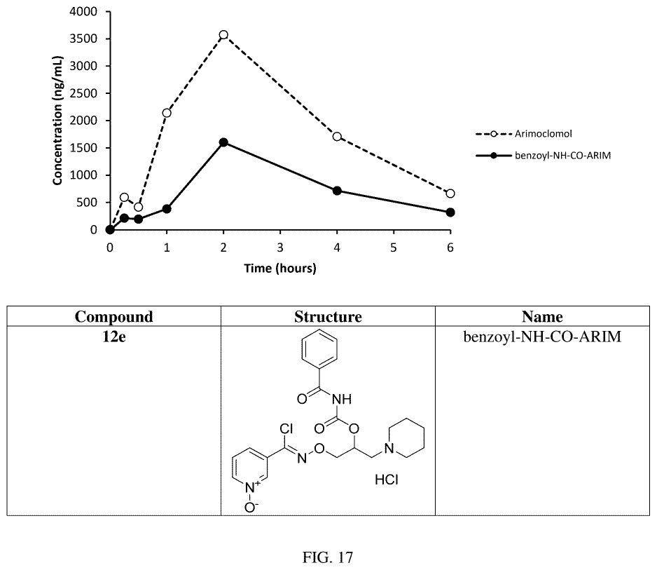

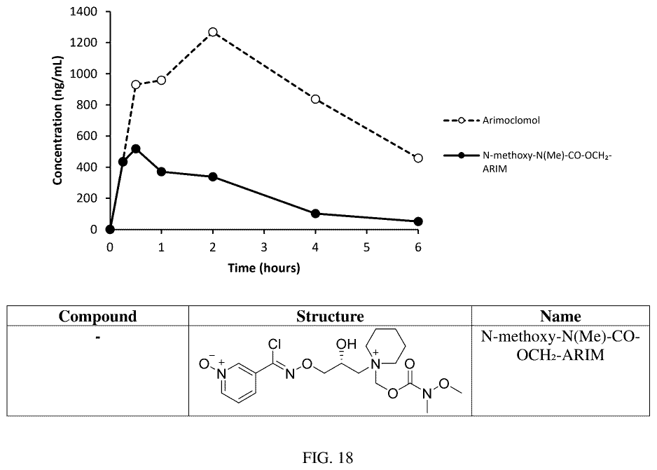

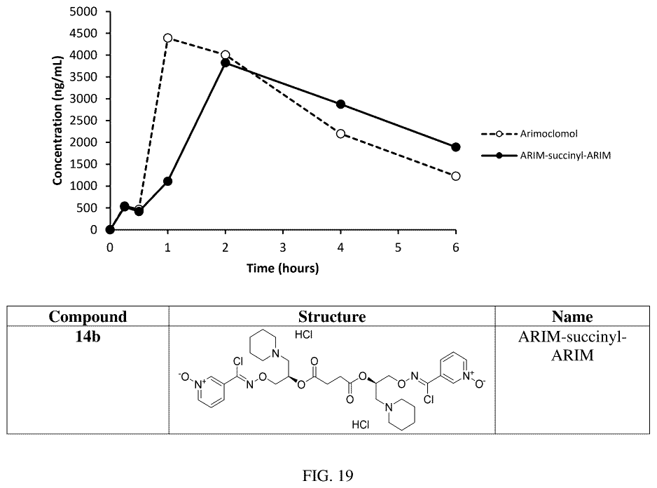

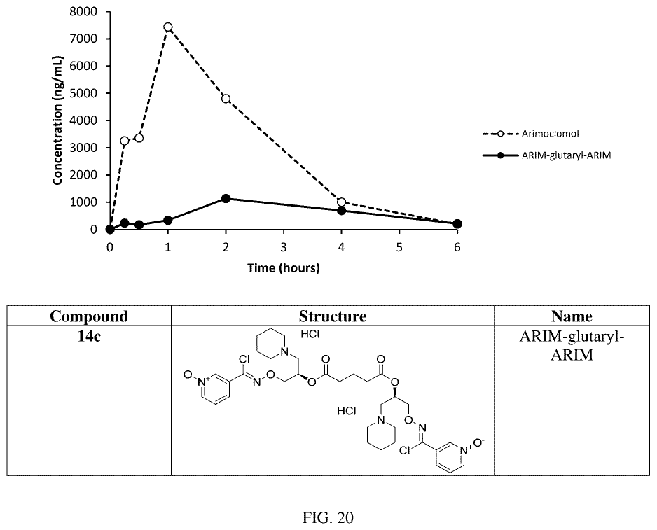

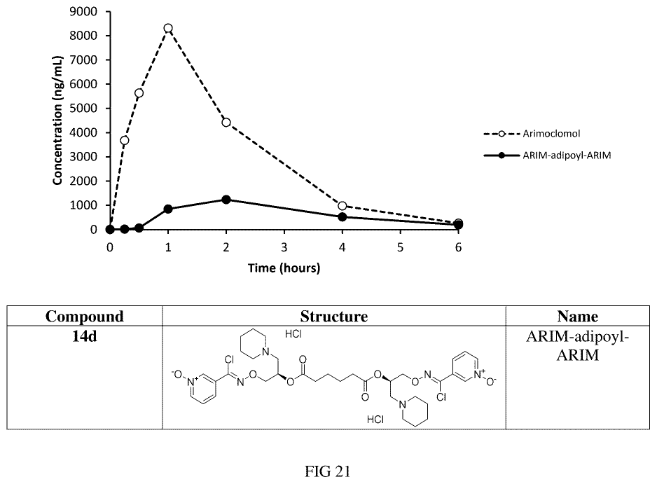

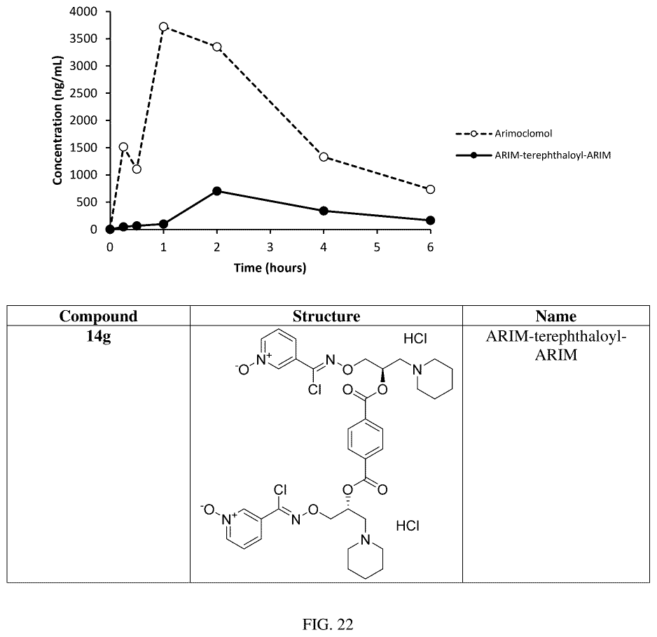

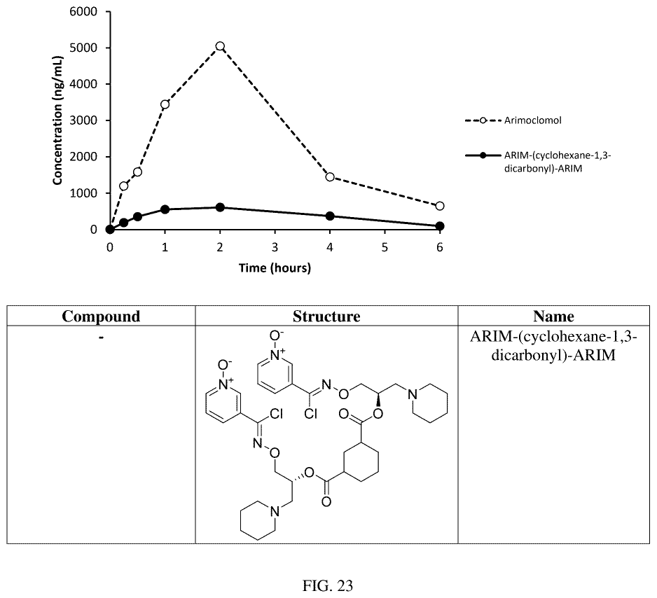

FIGS. 1-23 show the concentration of arimoclomol measured in plasma of rats for 6 hours after administration of various compounds described above compared to plasma concentrations measured in rats after administration or arimoclomol. A person skilled in the art will appreciate that the compounds named in FIG. 1-23 release arimoclomol free-base following hydrolysis and that the various conjugations modulate the rate of the hydrolysis.

### Example 2—Treatment of Niemann-Pick Disease, Type C, in Patients Under 2 Years of Age

### Study Design

Data regarding neonatal presentation of NPC is very limited. Although most patients present during childhood with one or more neurological manifestations, very early-onset patients are usually diagnosed based on isolated systemic manifestations [3]. NPC is a very rare disease and children from newborn to 2 years of age are considered particularly fragile and vulnerable. All patients in this paediatric substudy were to receive active treatment with arimoclomol as it was not considered ethical to expose these children to placebo.

Based on feasibility, 3-5 patients were expected to be enrolled during the recruitment period preferably at the open sites participating in the main study. The very low number of patients diagnosed in the neonatal and infantile period was confirmed in a paper where a medical center reported 10 patients with neonatal onset over a period of 22 years [3].

The selection criteria for this paediatric substudy were chosen to exclude newborns (less than 6 months of age) and children with perinatal diseases other than NPC which can cause severe development failure and to avoid interference with the assessment of safety of arimoclomol during the trial.

In addition to the scheduled study visits in the paediatric substudy, the investigator was encouraged to arrange for the patient to attend the center for unscheduled visits, performing the relevant assessments. These visits could be arranged at the discretion of the investigator at any point during the paediatric substudy, to closely follow and monitor the safety of the patients.

Arimoclomol dosing was based on the patient's actual weight at a given visit to determine a dose that was as accurate as possible and accounted for the expected weight gain during the conduct of the paediatric substudy. PK sampling was to take place to analyse AUC and confirm when the target AUC had been attained.

### Dosing Regimen and Preparation

Based on population PK simulations, a starting dose of 3.2 mg arimoclomol citrate/kg t.i.d. was selected for all patients 6 to less than 24 months of age.

One 100 mg capsule was to be opened per administration, and the full content of the capsule to be dispersed into 20 mL of water resulting in a suspension/solution with a concentration of 5 mg/mL. It was also optional to disperse the IMP suspension/solution in other liquids, such as apple juice, and soft foodstuff, e.g., yogurt or apple sauce. In the dispersed state, arimoclomol could also be administered via a gastric tube (if applicable). For full administration of the dose, the tube should be flushed with 5 mL of water. Arimoclomol was to be administered orally using a 10 mL syringe with 0.2 mL graduations. The appropriate amount was withdrawn according to the formula below and the calculated number of mLs was rounded down to nearest 0.2 mL. Arimoclomol was to be administered orally t.i.d. The patient's dose in mL is based on the patient's body weight in kg (measured with 1 decimal) and calculated using the equation:

$$Weight ( kg ) \times 3.2 mg kg / 5 mg ml = mL of solution to be withdrawn ( rounded down to nearest 0.2 mL ) ,$$

$$administered t . i . d .$$

The initial dose was based on 3.2 mg arimoclomol citrate/kg and the patient's body weight in kg. If the initial PK sample demonstrated that a dosing adjustment was needed, the adjusted dose should be applied (instead of 3.2 mg/kg) in the above equation going forward. If PK sampling needed to be repeated, for any reason, an unscheduled visit was to be performed.

Once an enrolled patient turned 24 months of age, the dosing of arimoclomol was to be based on Table 1 (similar to dosing in the main study). This change in dose had to occur as soon as possible but no later than the patient's next clinic visit. The patient's weight was measured at each visit and the IMP dose was adjusted as required. If required, arimoclomol could be dispersed in 10-20 mL (i.e., 1-2 tablespoons) liquid (water, apple juice) or soft foodstuff (yoghurt or apple sauce) or administered via a gastric tube. If 100 mg capsules in solution were still used after a patient turned 24 months of age, the amount of mL solution to administer t.i.d. is shown in Table 3:

TABLE 11
Arimoclomol Dosing Schedule According to Patient Bodyweight

| | | | Solution of |
|---|---|---|---|
| | | | 100 mg |
| | | | arimoclomol |
| | | Corresponding | citrate |
| Patient | | dose of | capsule in 20 |
| body | | arimoclomol | ml. liquid |
| weight | Arimoclomol citrate dose | base^{a} | (5 mg/ml.) |
| 8-15 kg | 50 mg t.i.d. (150 mg/day) | 31 mg tid. | 10 mL t.i.d. |
| >15-22 kg | 75 mg t.i.d. (225 mg/day) | 47 mg ti.d. | 15 mL t.i.d. |
| >22-38 kg | 100 mg t.i.d. (300 mg/day) | 62 mg t.i.d. | 20 mL t.i.d. |
| >38-55 kg | 150 mg ta.d. (450 mg/day) | 39 mg t.i.d. | 30 mL^{b} t.i.d. |
| >55 kg | 200 mg ta.d. (600 mg/day) | 124 mg t.i.d. | 40 mL^{b} t.i.d. |

t.i.d. = 3 times per day.
^{a}Conversion factor from arimoclomol citrate to arimoclomol free base: [citrate dose] * 0.620241.
^{b}Two 100 mg arimoclomol citrate capsules are dissolved in 40 mL liquid (5 mg/mL).

If a patient who was receiving 150 mg IMP per day (50 mg t.i.d.) required a dose reduction, a dose of 75 mg per day (25 mg t.i.d.) was to be administered. This corresponds to 5 mL solution of 100 mg arimoclomol citrate capsule in 20 mL liquid (5 mg/mL).

### PK Assessments

AUC_{0.8,SS} was determined from PK samples at Visit 2 to assess if the exposure level was acceptable and to describe PK in patients aged 6 to <24 months at study enrolment.

Additional PK data collected at Visits 6 and 8 were to be incorporated into a population PK model. PK samples were collected through venipuncture. At Visit 2, PK sampling to confirm dose selection was performed at 2 time-points within the time window of 6-8 hours following the first dose of arimoclomol with a minimum 0.25 hours between the 2 samples. PK sampling had to be performed prior to the second dose of arimoclomol. At Visits 6 and 8, PK samples for population PK were taken 5 min (±5 min) prior to the first daily dose of arimoclomol and 30 min (±5 min) following arimoclomol dosing. The results are presented in Table 2:

TABLE 12
PK data obtained from patients under 2 years of age.

| Patient | | Age | Bodyweight | Dose | CL | K_{TR} | K_{A} | AUC_{(0-8 h, ss)}_{^{a}} | C_{max, ss}_{^{a}} | C_{min, ss}_{^{a}} |
|---|---|---|---|---|---|---|---|---|---|---|
| No. | Visit | (years) | (kg) | (μg) | (L/h) | (1/h) | (1/h) | (h.ug/L) | (ug/L) | (ug/L) |
| 1 | 2 | 2.019 | 11.8 | 31,011.6 | 18.111 | 19.454 | 0.455 | 1,712.3 | 374.8 | 118.8 |
| | 8 | 3.038 | 13.3 | 31,011.6 | 19.473 | 19.454 | 0.458 | 1,592.5 | 357.4 | 106.5 |
| 2 | 2 | 1.212 | 7.65 | 15,183.28 | 8.357 | 20.8 | 0.339 | 1,816.9 | 294.2 | 173.3 |
| | 4 (+1) | 1.288 | 7.80 | 15,183.28 | 8.471 | 20.8 | 0.34 | 1,792.3 | 291.1 | 170.4 |
| | 4 (+2) | 1.327 | 7.75 | 15,183.28 | 8.461 | 20.8 | 0.34 | 1,794.4 | 291.4 | 170.6 |
| | 5 (+1) | 1.500 | 7.50 | 15,381.75 | 8.393 | 20.8 | 0.34 | 1,832.7 | 297.2 | 174.5 |
| | 6 | 1.731 | 8.30 | 20,219.56 | 8.893 | 20.8 | 0.341 | 2,273.7 | 373.1 | 213.6 |
| | 8 | 2.192 | 9.20 | 31,011.6 | 14.649 | 20.8 | 0.342 | 2,117.0 | 387.5 | 173.5 |
| 3 | 2 | 1.731 | 9.7 | 19,227.19 | 12.862 | 17.783 | 0.331 | 1,494.9 | 261.5 | 129.2 |
| | 6 | 2.212 | 11.1 | 31,011.6 | 21.525 | 17.783 | 0.332 | 1,440.7 | 283.8 | 105.8 |
| | 8 | 2.654 | 12.0 | 31,011.6 | 22.5 | 17.783 | 0.333 | 1,378.3 | 274.7 | 99.6 |
| 4 | 2 | 1.404 | 9.97 | 19,227.19 | 6.758 | 24.073 | 0.299 | 2,844.8 | 431.5 | 288.4 |
| | 6 | 1.904 | 10.5 | 21,087.89 | 7.057 | 24.073 | 0.3 | 2,988 | 456.7 | 300.4 |
| | 8 | 2.385 | 11.0 | 31,011.6 | 11.282 | 24.073 | 0.301 | 2,748.8 | 457 | 249.6 |
| 5 | 2 | 1.615 | 10.3 | 19,847.42 | 9.081 | 17.204 | 0.289 | 2,185.5 | 343.5 | 212.1 |

^{a}Simulated values have units referring to arimoclomol freebase.
CL = clearance; K_{TR} = transit compartment rate constant; K_{A} = absorption rate constant; AUC_{(0-8 h), ss} = area under the plasma concentration curve from time 0 to 8 hours at steady state; C_{max, ss} = maximum plasma concentration at steady state; C_{min, ss} = minimum plasma concentration at steady state.

The population PK model's ability to predict arimoclomol concentrations for each patient was assessed with a range visual predictive check (VPC) which compared the observed data with simulated concentration-time profiles for 1000 virtual patients of equal age and weight profiles for the study duration. For 3 of the patients (Patient No. 2, 4, and 5) simulations from the model were consistent with observed data. For the 2 remaining patients (Patient No. 1 and 3) the observations were below the lower limit of the 90% prediction interval at visit 2, whereas improved consistency between predictions and observations were seen at later visits.

### Bayley III Scale Scoring

The Bailey III score, a three-part scale for assessing the developmental delay of children, was used to assess the development of the patients. It was implemented as it targets children between the age of 1 months and 42 months.

The Bayley III score was assessed by means of the Bayley Scales of Infant Development—Third Edition (BSID-III). This child development assessment tool was used for the following reasons: universality of use; availability of age-equivalent scores for severely impaired children; nonverbal content as on the cognitive scale. Also, the BSID-III is deemed valid as test instrument in children from cultures in western countries. The BSID-III assesses developmental functioning in infants and young children aged 1-42 months. The test consists of scores in 5 domains of function in infants: cognitive, communication, physical, social/emotional, and adaptive behavior. The following domains could be administered independently: cognitive, language, and motor scales as sub scores. The total score as well as these sub-scores were recorded separately. The BSID-III assessment was also applicable for pediatric substudy patients older than 42 months. This was justified by significant developmental delay in the patient population including all domains monitored by the scoring tool. The BSID-III was only to be administered by trained professionals who had experience and training in the test. As the test situation could be a burden for the patient it was recommended to start with the motor and cognitive scales and only continue with language if possible. The scales for social/emotional and adaptive behavior were conducted with parent questionnaires.

### Patient No. 1

With scaled scores, the patient appeared to be below average development compared to age expectations with regards to the cognitive domain and the expressive communication, receptive communication, fine motor and gross motor subscales at baseline and declined further for all but the gross motor subscale during the study. The change in GSVs indicated that the patient was largely stable in the cognitive domain, expressive communication, fine motor and gross motor subscales from baseline to visit 6, after which decline in all domains/subscales were seen through visit. However, for receptive communication the pattern varied more with an initial decline at visit 6, then a return to baseline level at visit 8 after which a decline was recorded at visit 9.

Overall, the patient declined in functioning over the course of study participation.

### Patient No. 2

This patient appeared to be below average development compared to age expectations with regards to the cognitive domain and the expressive communication, receptive communication, and gross motor subscales at baseline. For the fine motor subscale, the patient started out within age expectations at baseline but declined subsequently. The change in GSVs indicated that the patient was largely stable in all domains and subscales assessed from baseline to visit 11, and even with slight improvements in expressive and receptive communication from visit 9 and 10, respectively. Substantial and marked decline were seen for all domain and subscales, except receptive communication, between visits 11 and 12. However, notes at visit 12 indicate that the patient was tired and did not participate fully in the assessment, suggesting that this decline in scores was likely an artifact rather than a meaningful regression. The patient generally appeared to have stable developmental function over the course of study participation.

### Patient No. 3

At baseline, scaled scores for this patient indicated broadly age-appropriate developmental function for all domain and subscales assessed. For the gross motor subscale, a mild and gradual decrease in scaled scores falling below age expectations at visits 8 through 10 was seen. However, changes in GSVs indicated consistent improvements for all domains and subscales during the substudy compared to baseline levels. Overall, the patient appeared to gain developmental skills over the course of the substudy.

### Patient No. 4

Baseline scaled scores indicated age-appropriate abilities for the cognitive domain, receptive communication and fine motor subscales, and underdeveloped abilities compared to age expectations for the expressive communication and gross motor subscales. Data were only available from 2 visits (visit 2 and 8), however, changes in GSVs indicated modest improvement in the cognitive domain, and marked improvements in expressive communication and gross motor subscales from visit 2 to visit 8, while fine motor suggested stable abilities and receptive communication abilities declined from visit 2 to visit 8. Although limited data are available, the patient appeared with largely stable and potentially improved developmental function over the course of the substudy.

### Patient No. 5

Only baseline (visit 2) Bayley III scores were available for this patient, thus GSVs could not be calculated. Scaled scores for expressive communication, receptive communication, fine motor and gross motor subscales suggested average abilities compared to age expectations at baseline. Scaled scores for the cognitive domain suggested underdeveloped functioning compared to age expectations. Overall, the patient appeared to have broadly age-appropriate developmental function at baseline.

In summary, the evaluation of Bayley III scores indicated that 2 of the patients (Patient No. 3 and 5) were generally within the age-appropriate development window at baseline whereas 2 of the patients were generally underdeveloped compared to age expectations at baseline (Patient No. 1 and 2). The remaining patient (Patient No. 4) was age appropriate with regards to the cognitive domain, receptive communication, and fine motor subscales, and underdeveloped in the gross motor and expressive communication subscales at baseline. During the pediatric substudy, 1 patient gained developmental skills (Patient No. 3), 2 patients were largely stable over the course of the substudy (Patient No. 2 and 4), 1 patient generally declined over the course of the substudy (Patient ID 9901), and 1 patient only had data available from the baseline visit (Patient No. 5).

### 10.1 Disposition of Patients

During the recruitment period (30 Oct. 2018-24 Jan. 2024), 6 patients were screened for eligibility: 1 patient was a screening failure and 5 patients were enrolled in the pediatric substudy. 4 of the 5 patients participated in the substudy for more than 12 months. Of the 5 patients enrolled, 2 completed the substudy and 3 were withdrawn prior to the planned end of study visit. For 2 of these patients (Patient No. 1 and 4) their withdrawal from trial was due to withdrawal of consent by their LAR after 1029 and 483 days in the substudy, respectively. For the last patient (Patient No. 5), the criteria for discontinuation of arimoclomol treatment was met due to AEs, the treatment interruption exceeded 4 weeks and the patient was later withdrawn from the substudy after 128 days.

Overall, change in Bayley III scores (growth scale values) provided an indication of individual performance compared to baseline. During the substudy, 1 patient gained developmental skills, 2 patients were largely stable, and 1 patient generally declined. Results were inconclusive for 1 patient as Bayley III was only assessed at 1 timepoint.

What is claimed:

1. A compound having a structure of Formula I:

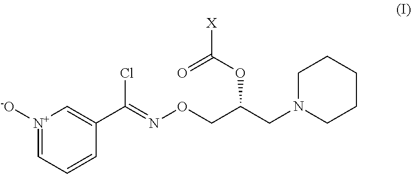

wherein X is selected from the group consisting of R, [O—R], [NR¹R²], and [NRH]
wherein O is oxygen, N is nitrogen, H is hydrogen, and
R, R¹ and R² are independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol,
or a pharmaceutically acceptable salt thereof.

2. The compound of 1, wherein the compound is a compound having a structure of Formula IA:

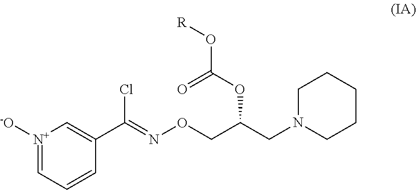

wherein R is selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

3. The compound of 2, wherein the compound of Formula IA has a structure selected from the group consisting of:

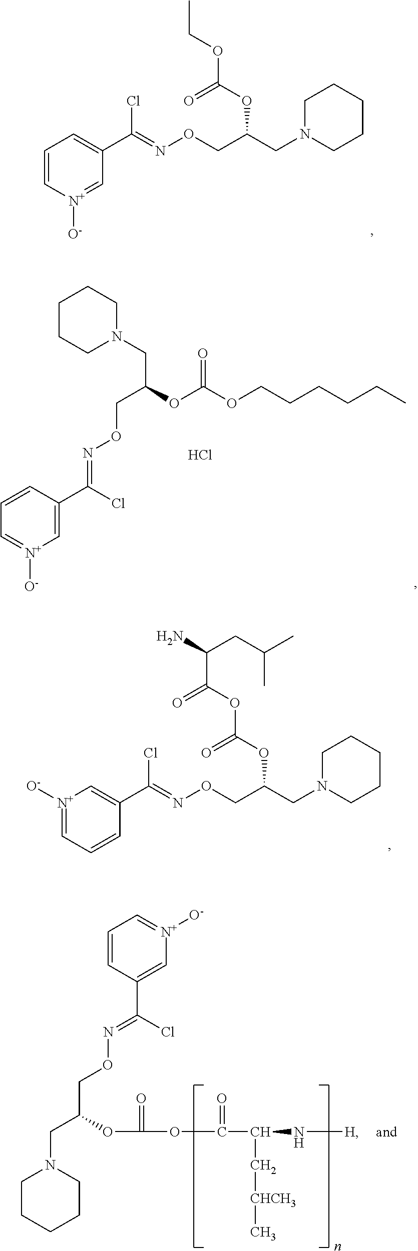

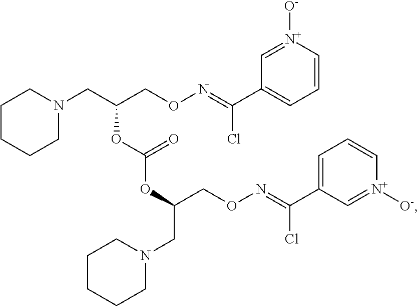

wherein n is 1-50;
or a pharmaceutically acceptable salt thereof.

4. The compound of 1, wherein the compound is a compound having a structure of Formula IB:

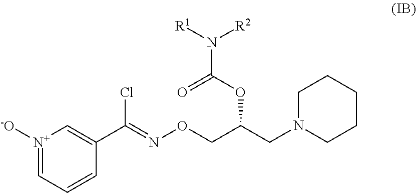

wherein R¹ and R² are independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

5. The compound of 4, wherein the compound of Formula IB has a structure selected from the group consisting of:

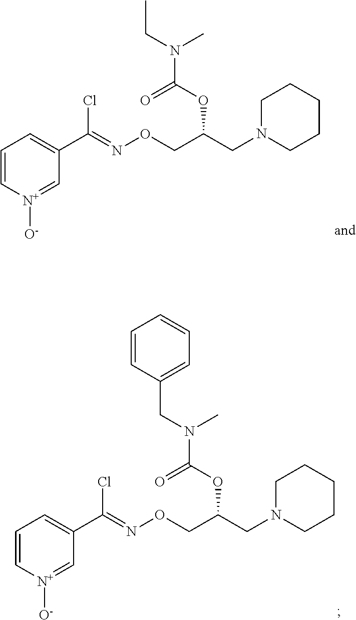 [SMILES: CCN(C)C(=O)O[C@@H](CO/N=C(\Cl)c1ccc[n+]([O-])c1)CN1CCCCC1 ; CN(Cc1ccccc1)C(=O)O[C@@H](CO/N=C(\Cl)c1ccc[n+]([O-])c1)CN1CCCCC1]

or a pharmaceutically acceptable salt thereof.

6. The compound of 1, wherein the compound of Formula I is a compound of Formula IC:

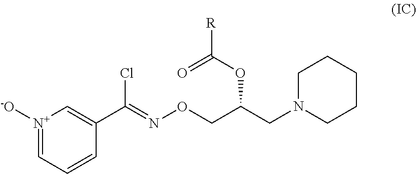

wherein R is selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

7. The compound of claim 6, wherein the compound of Formula IC is selected from the group consisting of:

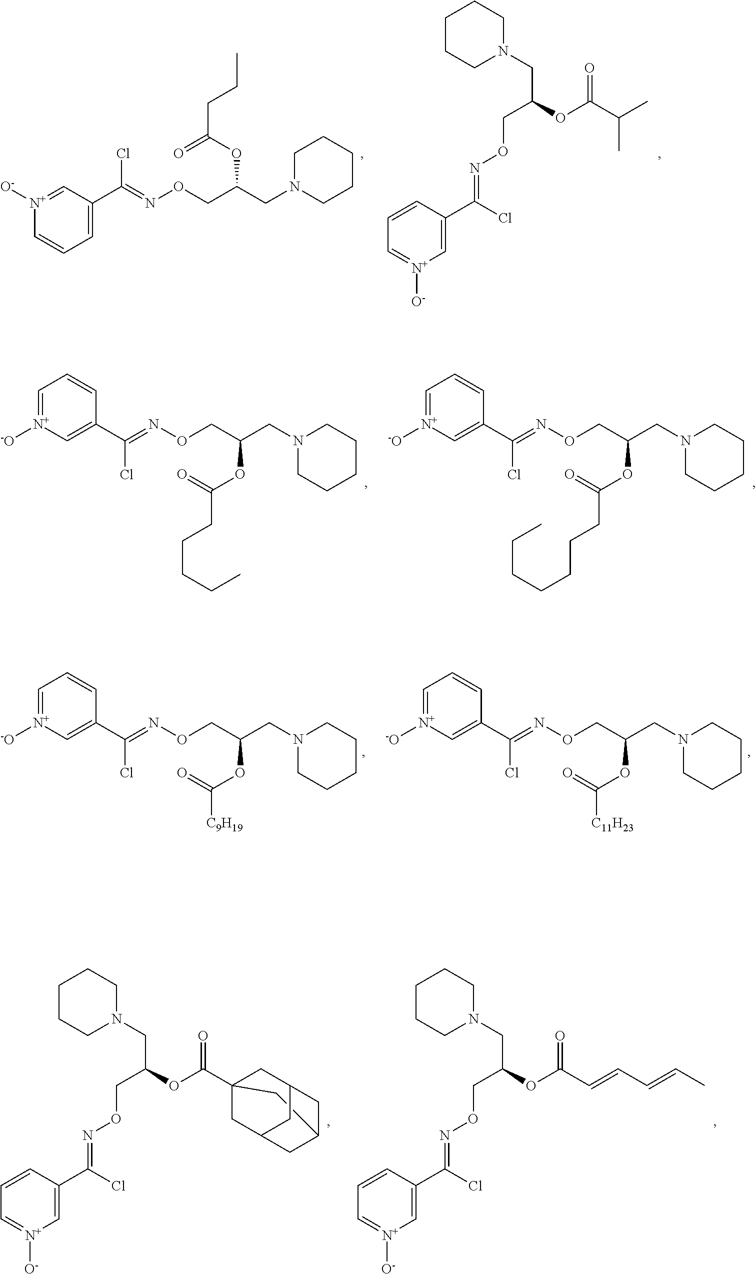

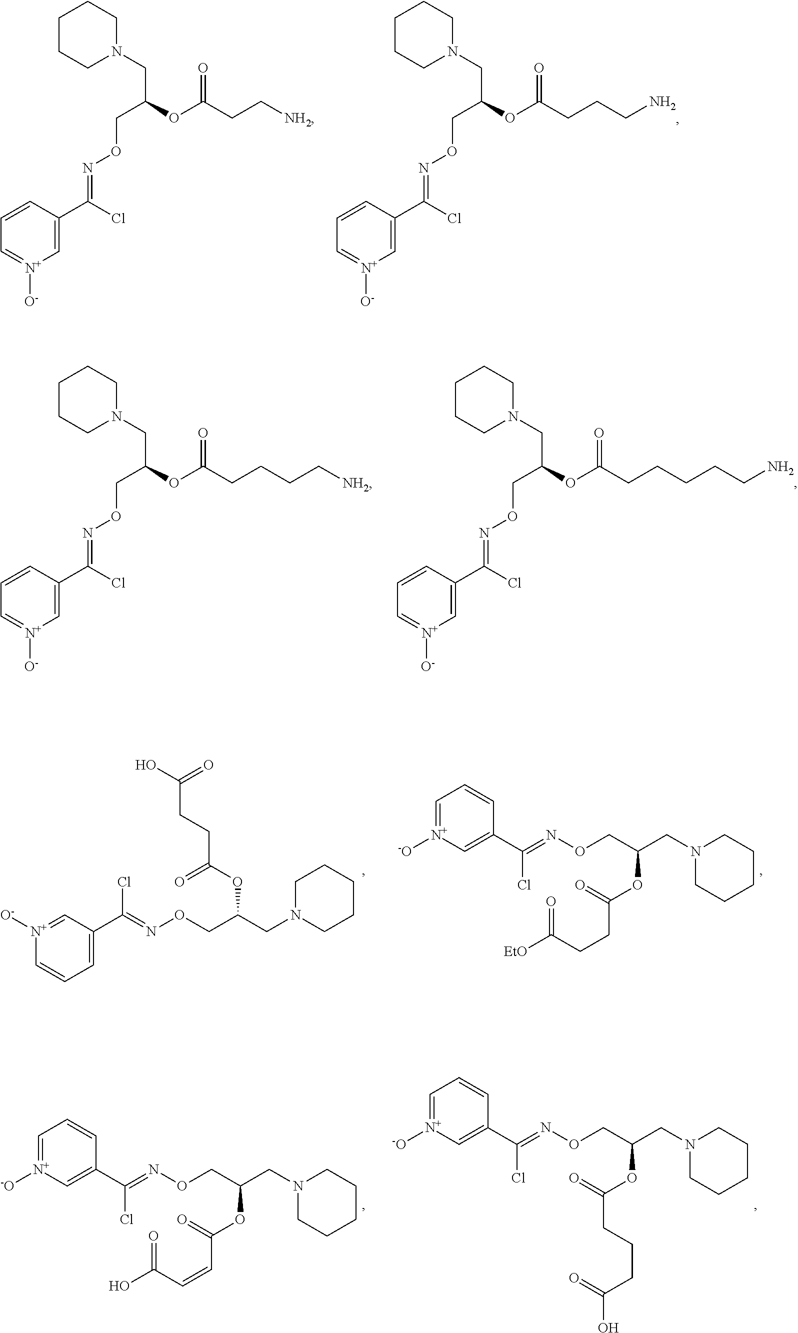

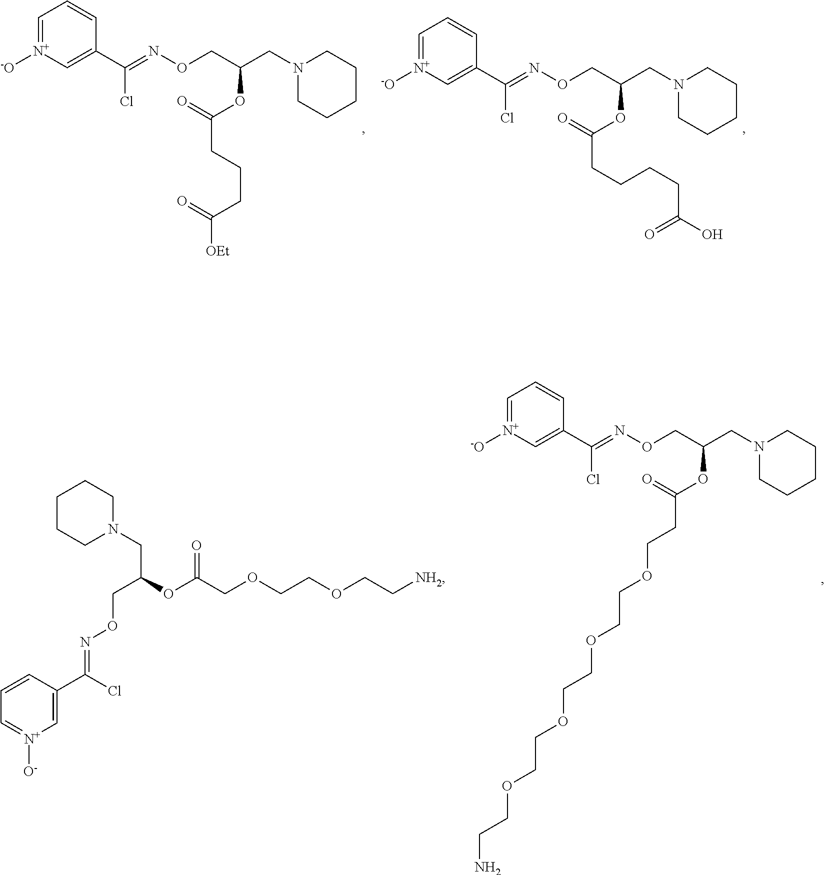

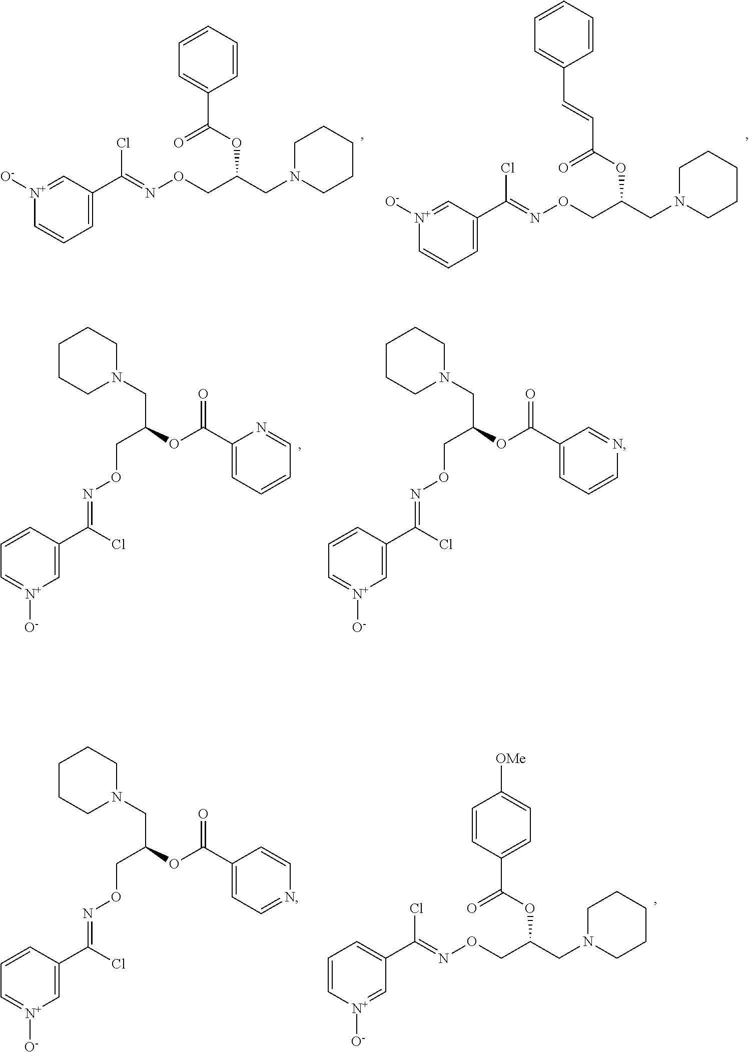

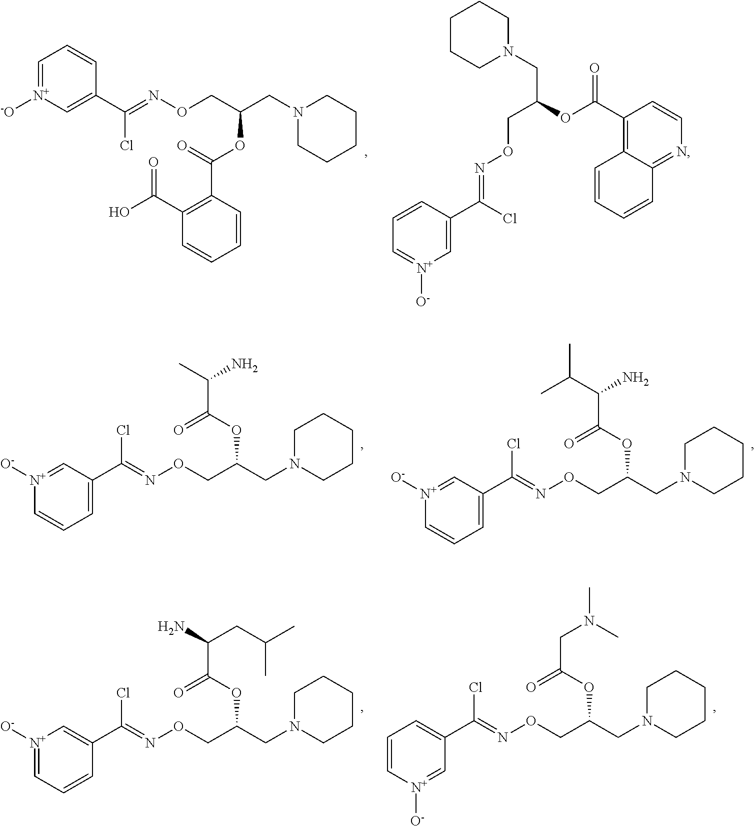

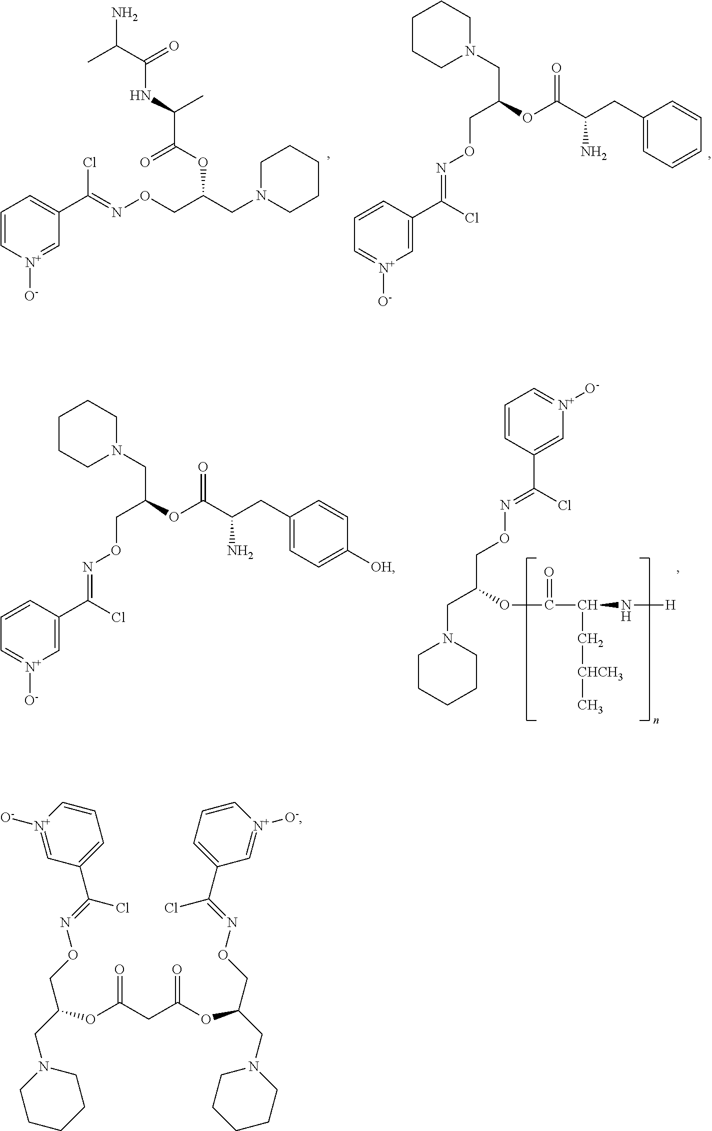

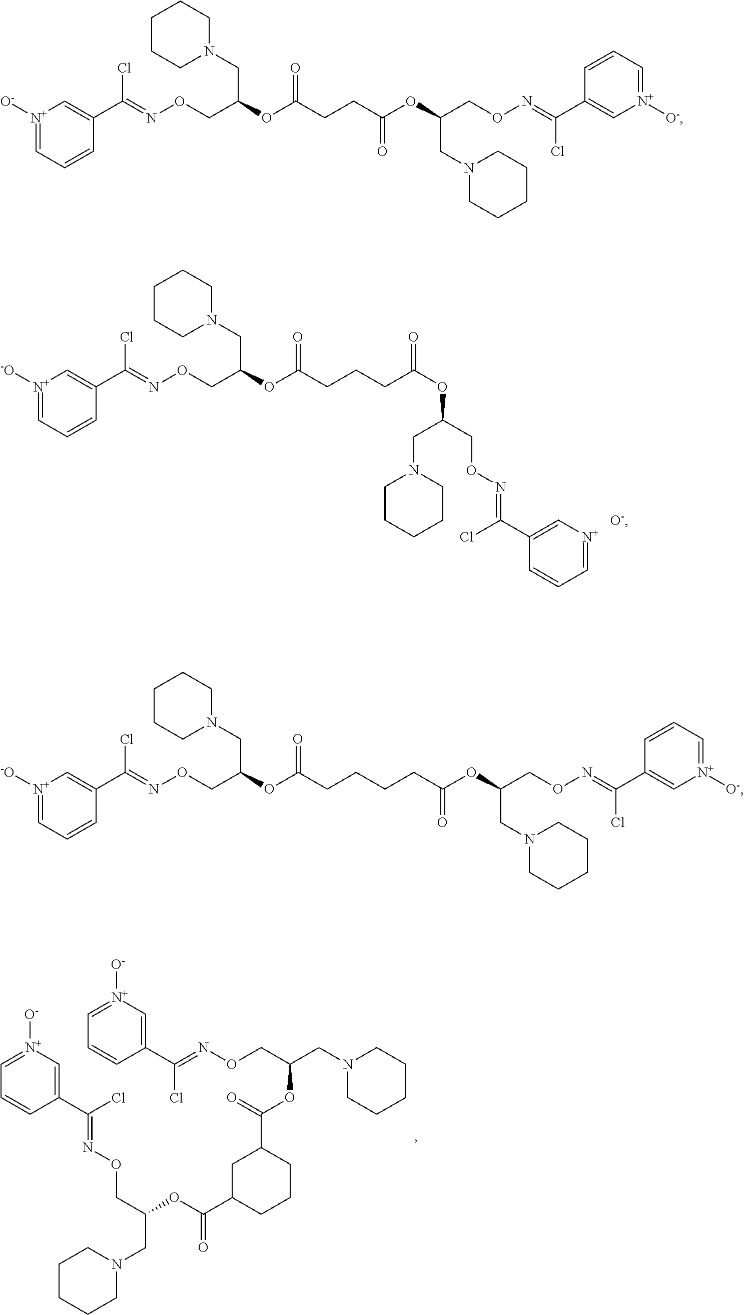

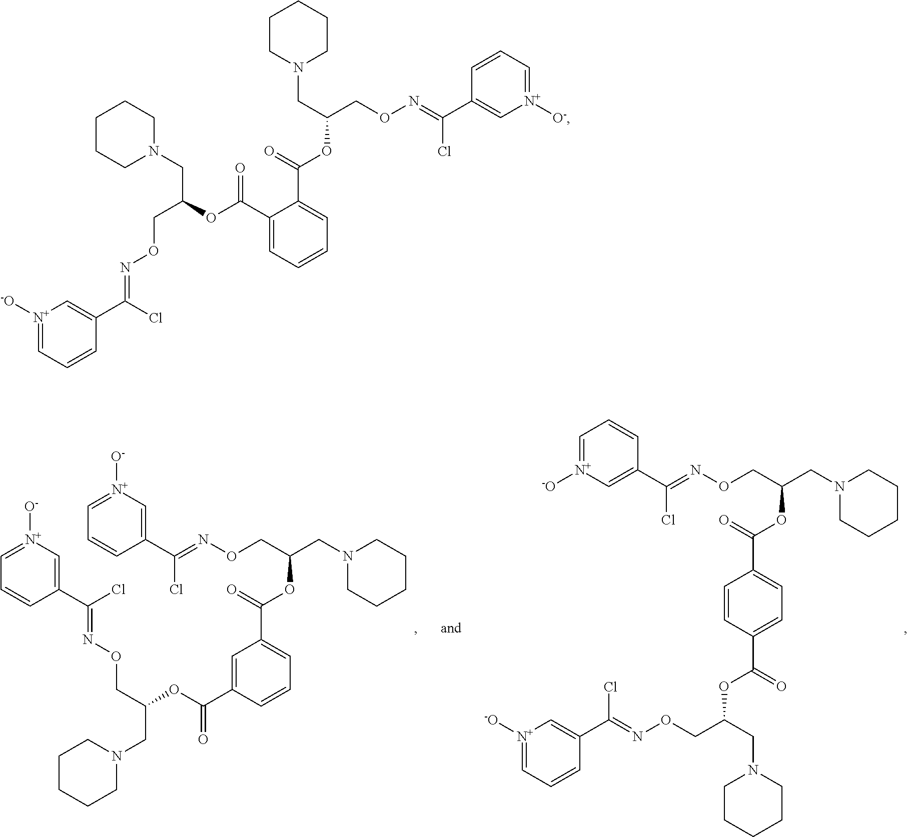

wherein n is 1-50;
or a pharmaceutically acceptable salt thereof.

8. The compound of 7, wherein the compound of Formula IC has a structure selected from the group consisting of:

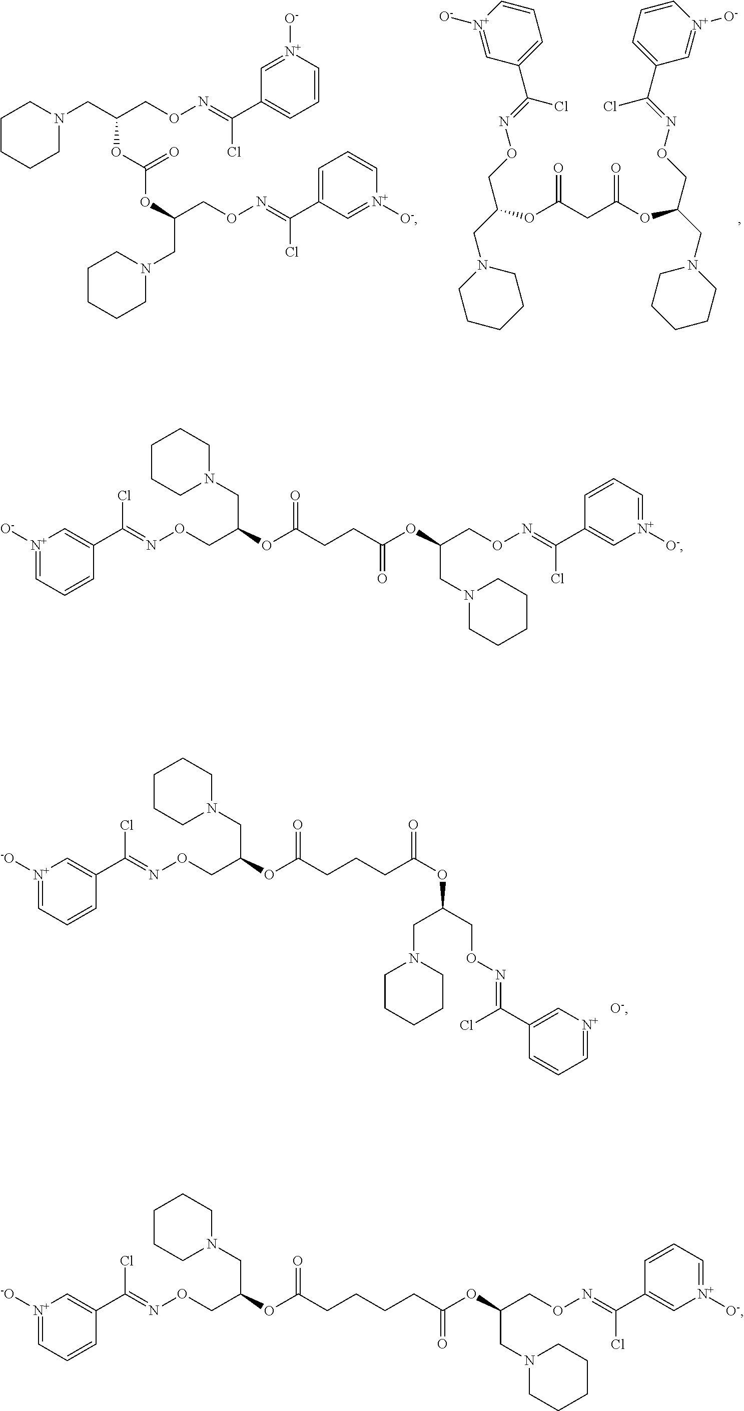

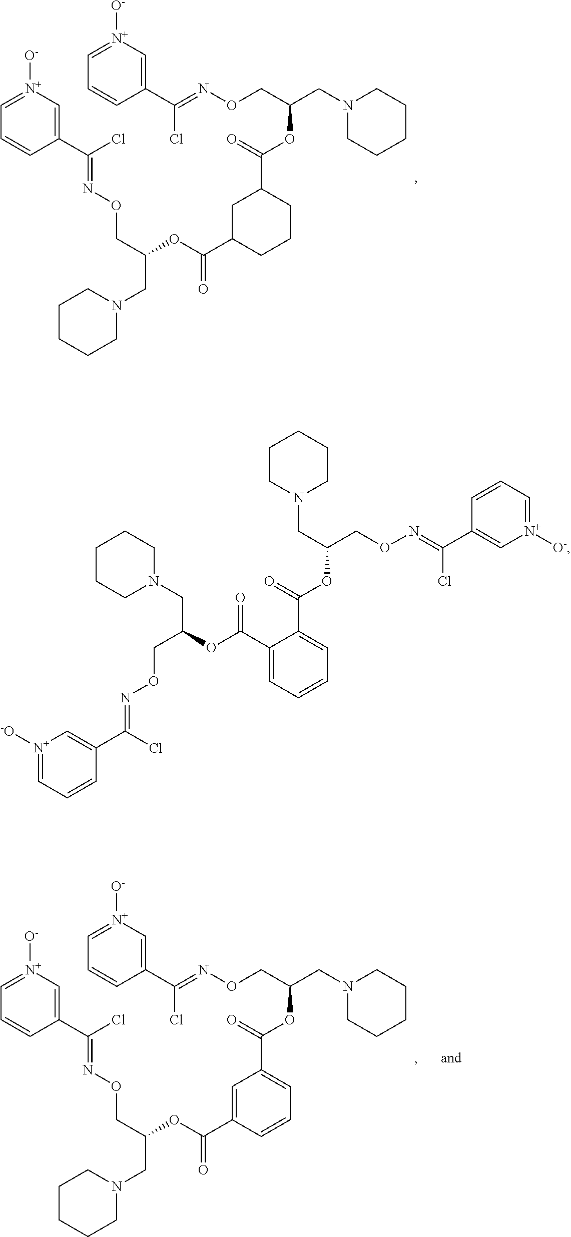

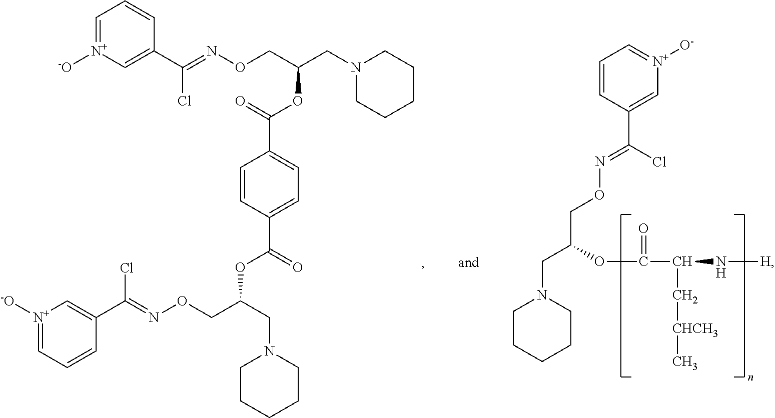

wherein n is 1-50;
or a pharmaceutically acceptable salt thereof.

9. The compound of 1, wherein the compound of Formula I is a compound of Formula ID:

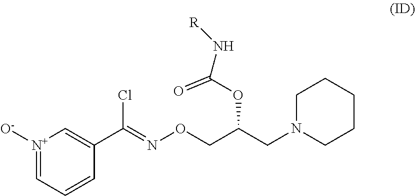

wherein R is selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

10. The compound of 9, wherein the compound of Formula ID has a structure selected from the group consisting of:

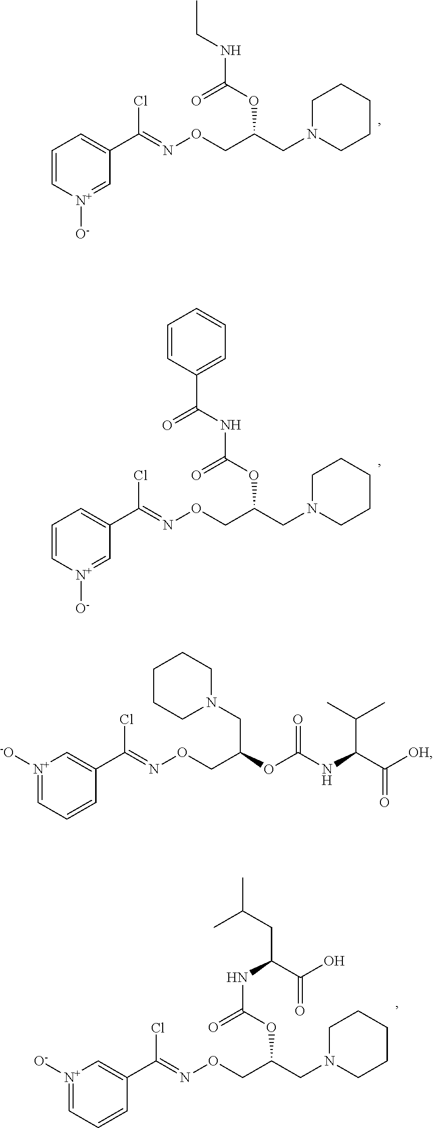

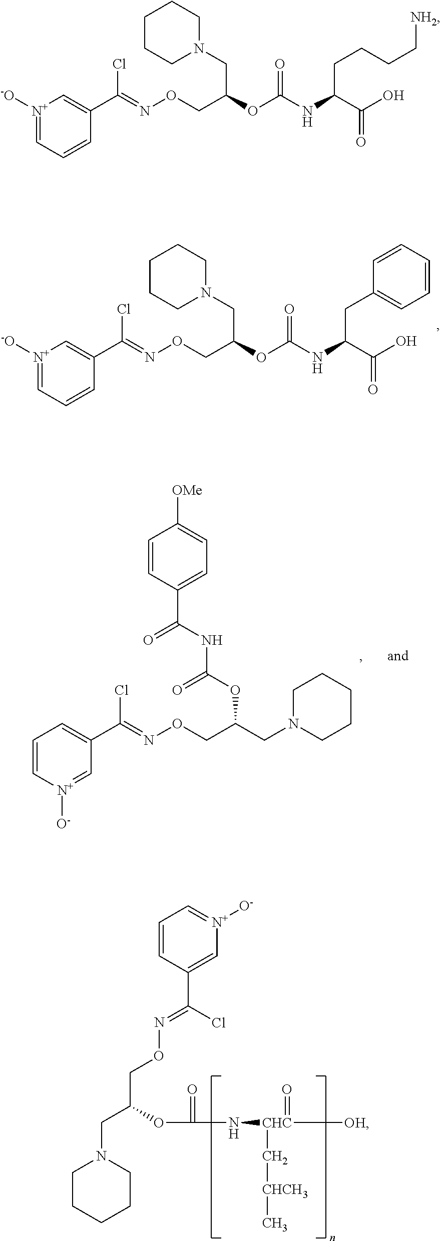

wherein n is 1-50;
or a pharmaceutically acceptable salt thereof.

11. A compound having a structure of Formula II:

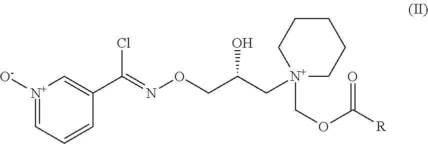

wherein R¹ and R² are independently selected from the group consisting of H, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

12. The compound of 11, wherein the compound of Formula II is selected from the group consisting of

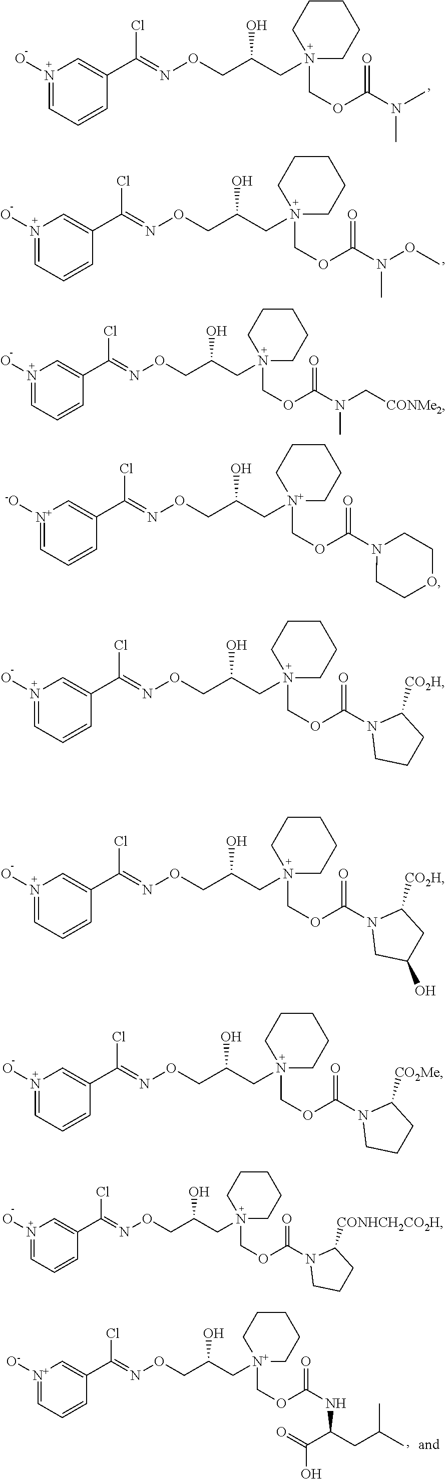

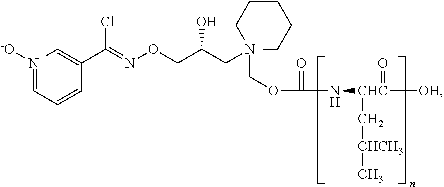

wherein n is 1-50;
or a pharmaceutically acceptable salt thereof.

13. A compound having a structure of Formula III:

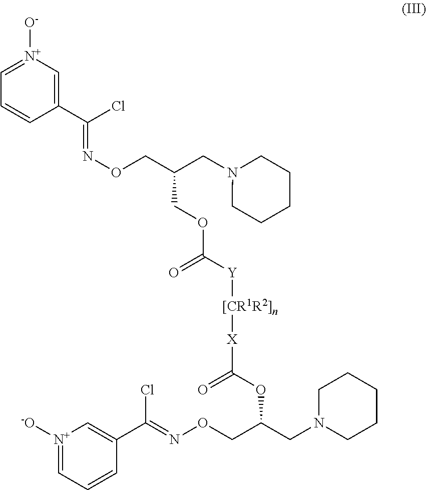

wherein X is selected from the group consisting of O, N, or absent,
Y is selected from the group consisting of O, N, or absent,
n is 1-10,
R¹ and R² are selected independently from each other and for each CR¹R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

14. A compound of Formula IV:

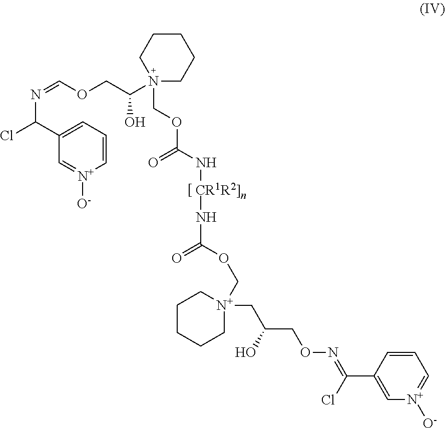

n is 1-10,
R¹ and R² are selected independently from each other and for each CR₁R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

15. A compound of Formula V

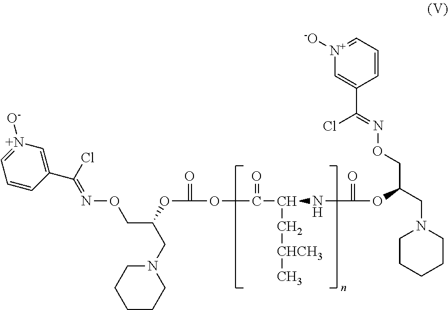

wherein n is 1-50,
or a pharmaceutically acceptable salt thereof.

16. A compound of Formula VI:

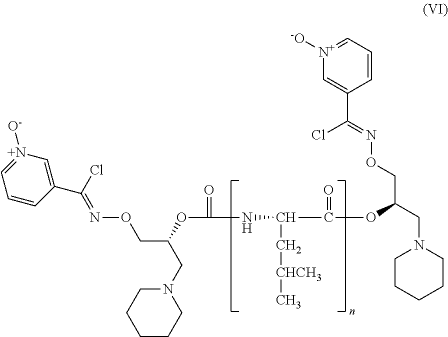

wherein n is 1-50;
or a pharmaceutically acceptable salt thereof.

17. A compound of Formula VII:

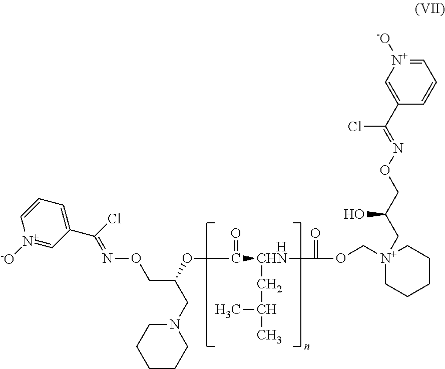

wherein n is 1-50,
or a pharmaceutically acceptable salt thereof.

18. A compound having a structure selected from the group consisting of:

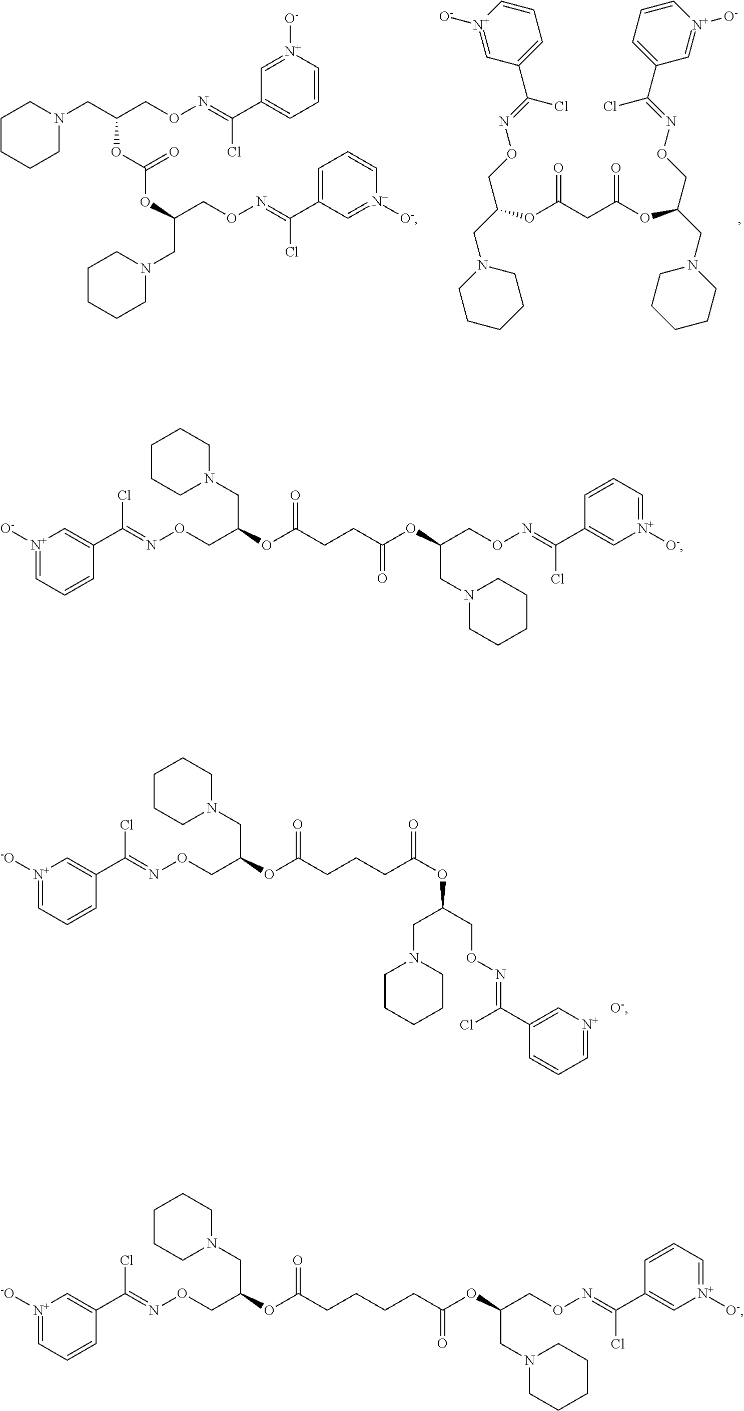

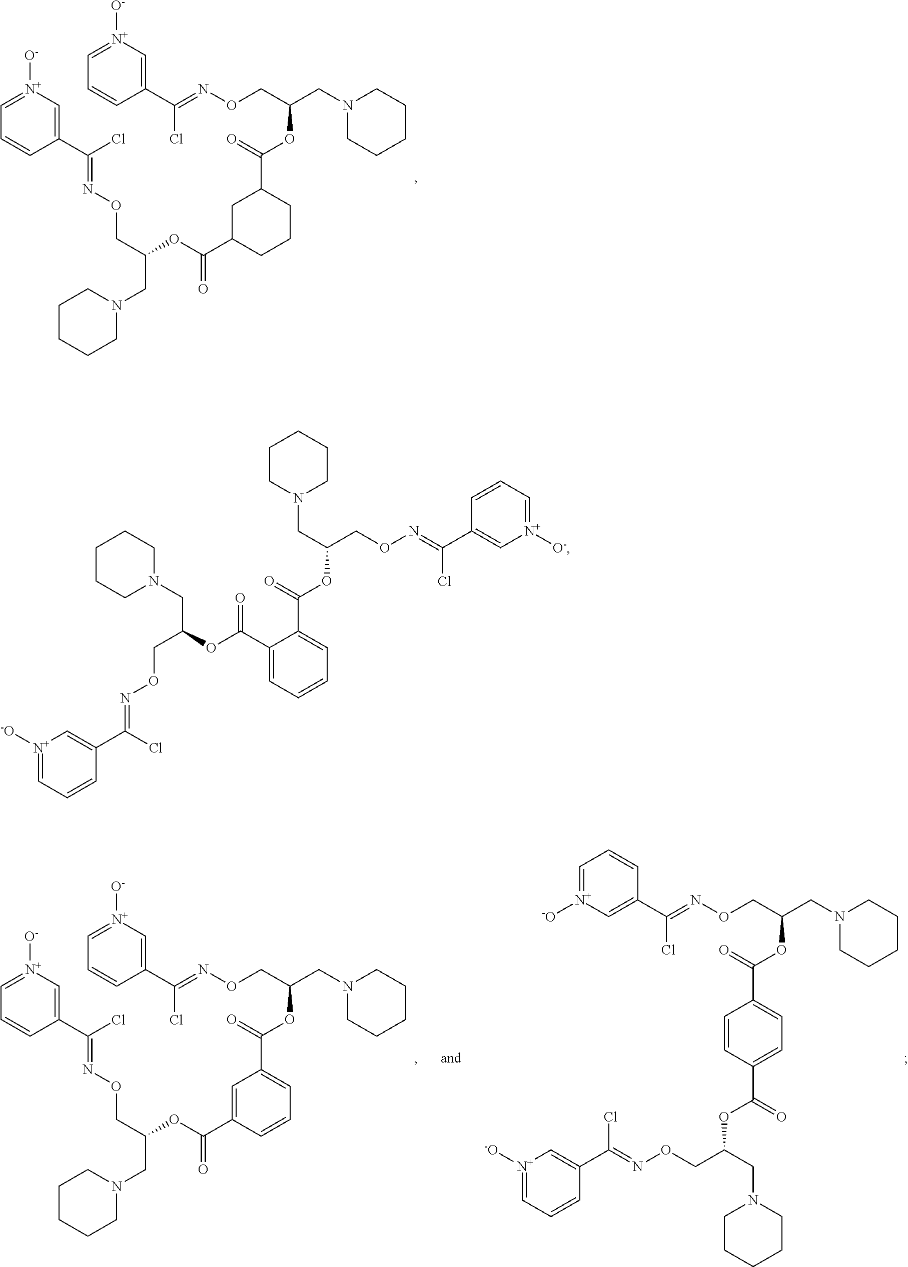

or a pharmaceutically acceptable salt thereof.

19. A compound selected from the group consisting of:

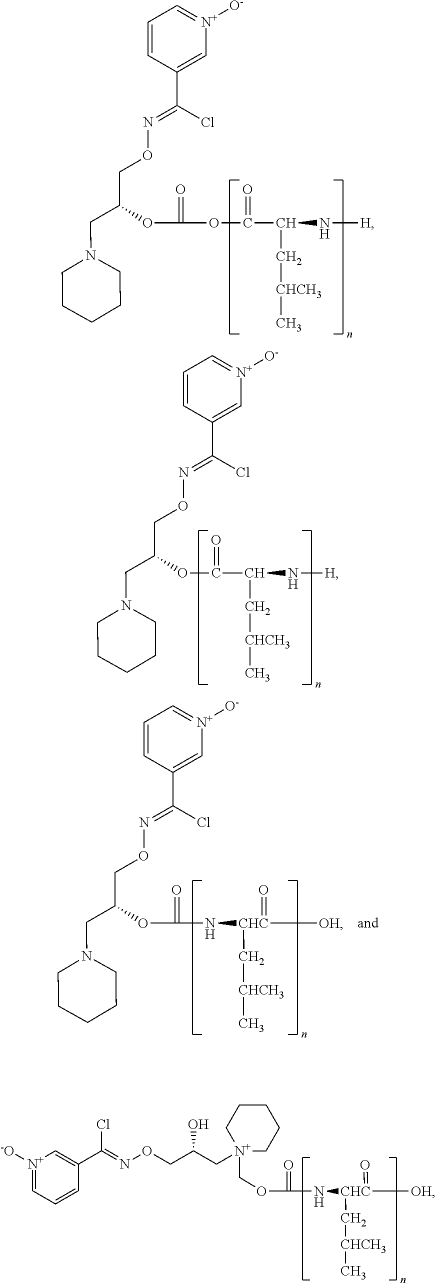

wherein n is 1-50;
or a pharmaceutically acceptable salt thereof.

20. The compound of claims 1-19, wherein the pharmaceutically acceptable salt is selected from: sulfate, citrate, acetate, oxalate, chloride, hydrochloride, bromide, hydrobromide, iodide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

21. A composition comprising:
a therapeutically effective amount of a compound of Formula I:

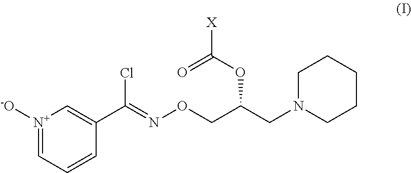

wherein X is selected from the group consisting of R, [O—R], [NR¹R²], and [NRH], where O is oxygen, N is nitrogen, and H is hydrogen;
R, R¹ and R² are independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

22. The composition of 21, wherein the compound is a compound having a structure of Formula IA:

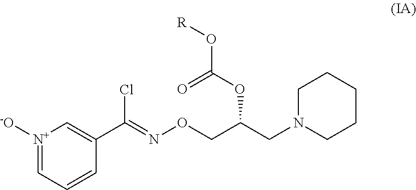

wherein R is selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

23. The composition of 22, wherein the compound of Formula IA is a compound having a structure selected from the group consisting of:

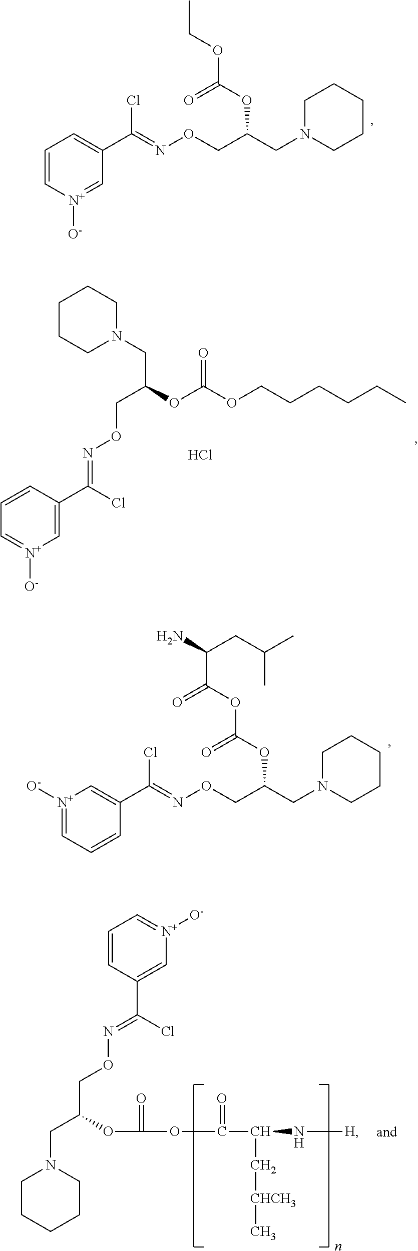

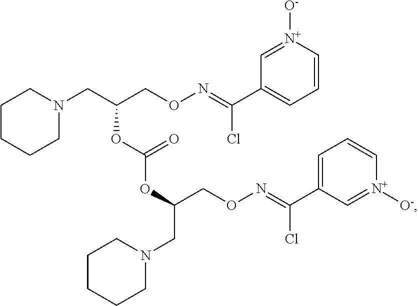

wherein n is 1-50;
or a pharmaceutically acceptable salt thereof.

24. The composition of 21, wherein the compound of Formula I is a compound having a structure of Formula IB:

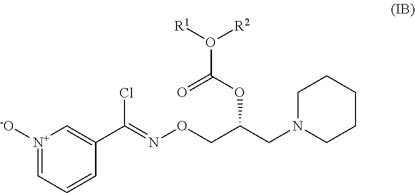

wherein R¹ and R₂ are independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

25. The composition of 24, wherein the compound of Formula IB is a compound having a structure selected from the group consisting of:

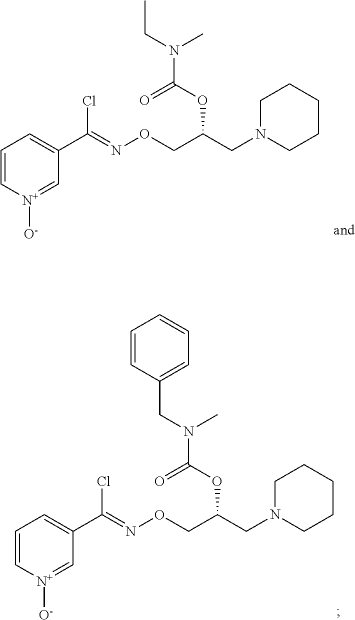 [SMILES: CCN(C)C(=O)O[C@@H](CO/N=C(\Cl)c1ccc[n+]([O-])c1)CN1CCCCC1 ; CN(Cc1ccccc1)C(=O)O[C@@H](CO/N=C(\Cl)c1ccc[n+]([O-])c1)CN1CCCCC1]

or a pharmaceutically acceptable salt thereof.

26. The composition of 21, wherein the compound of Formula I is a compound of Formula IC:

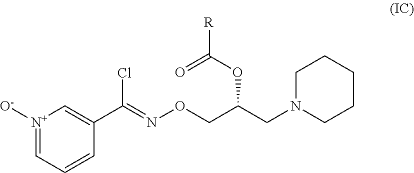

wherein R is selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

27. The composition of 26, wherein the compound of Formula IC is a compound having a structure selected from the group consisting of:

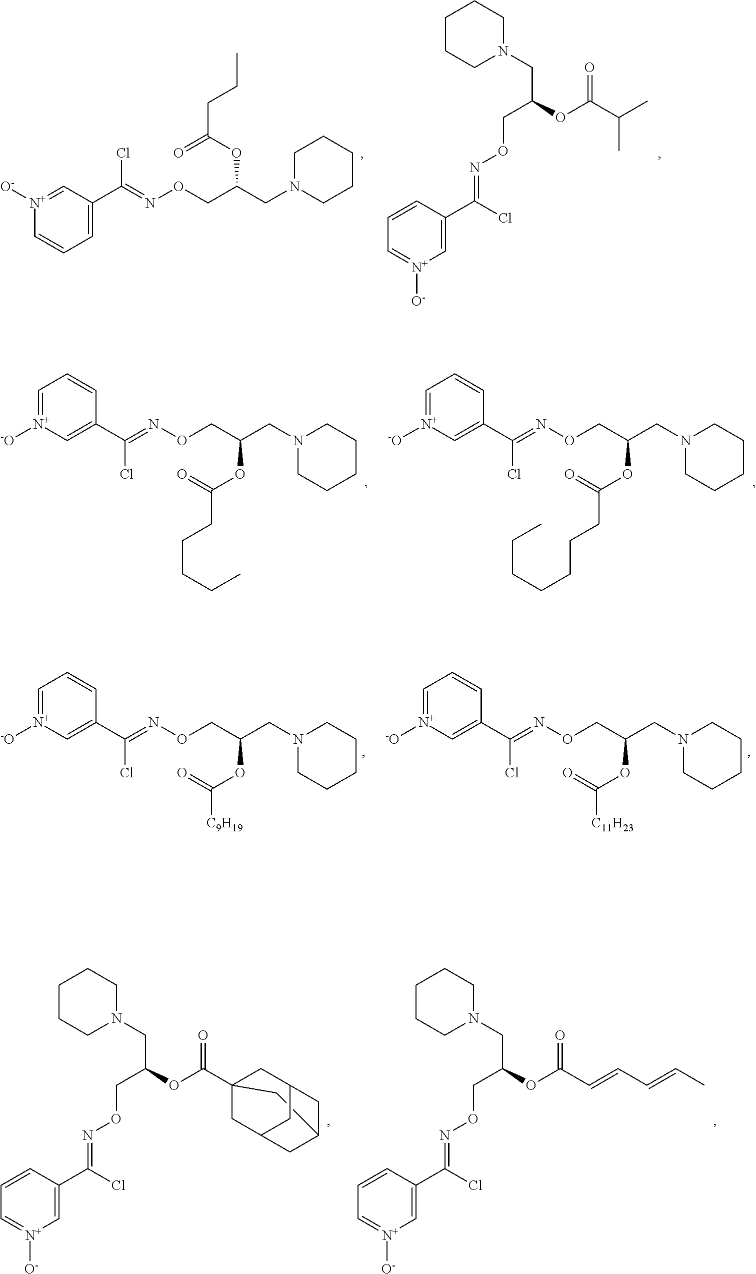

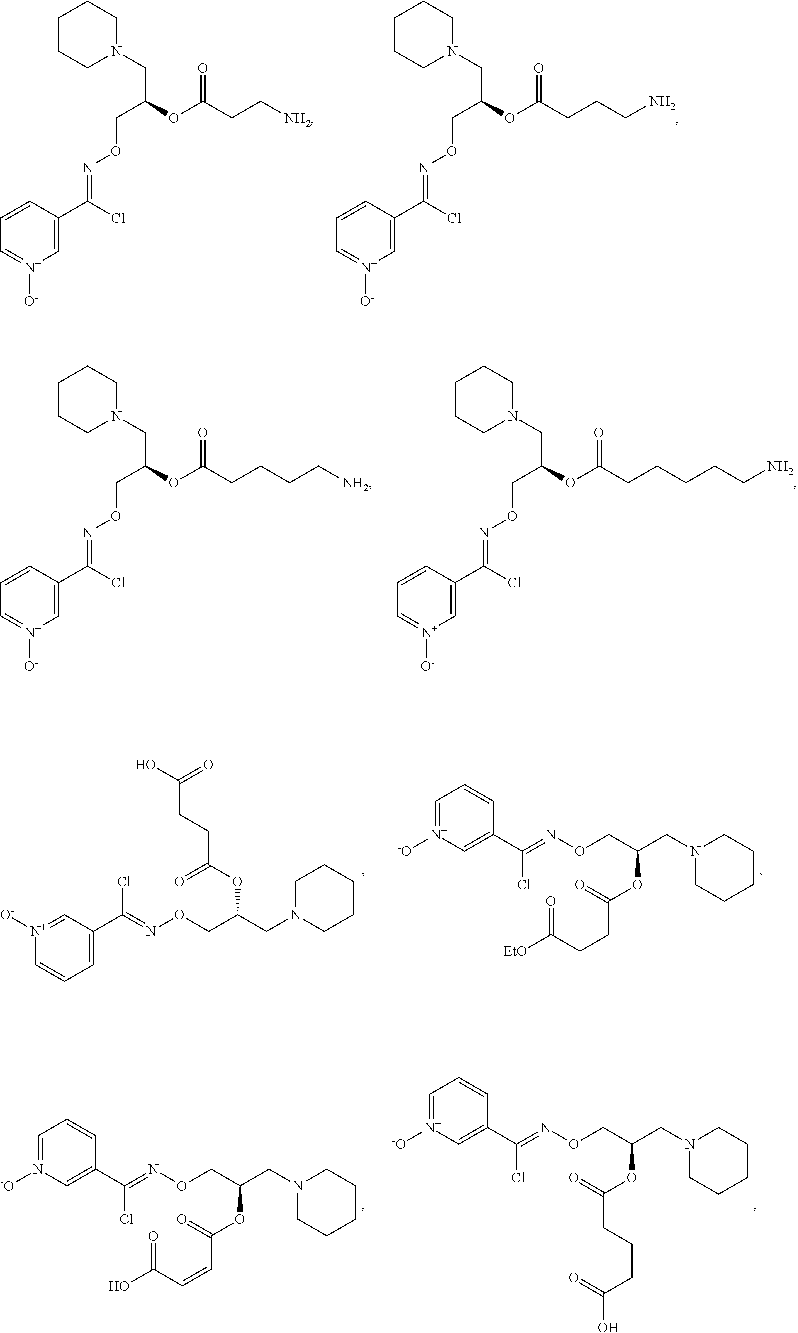

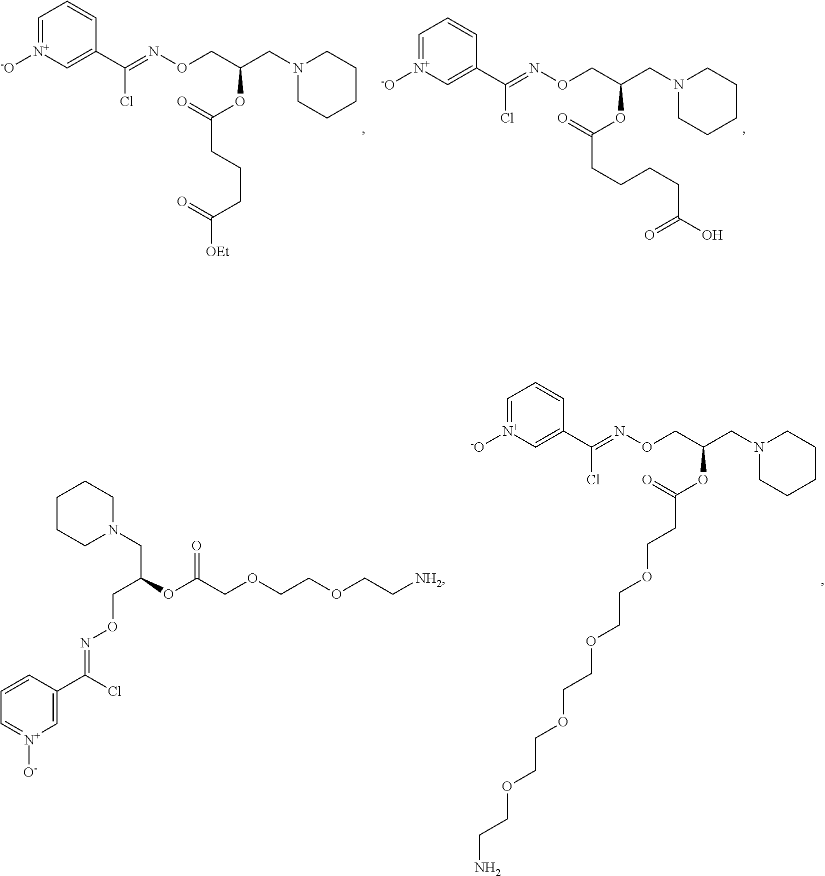

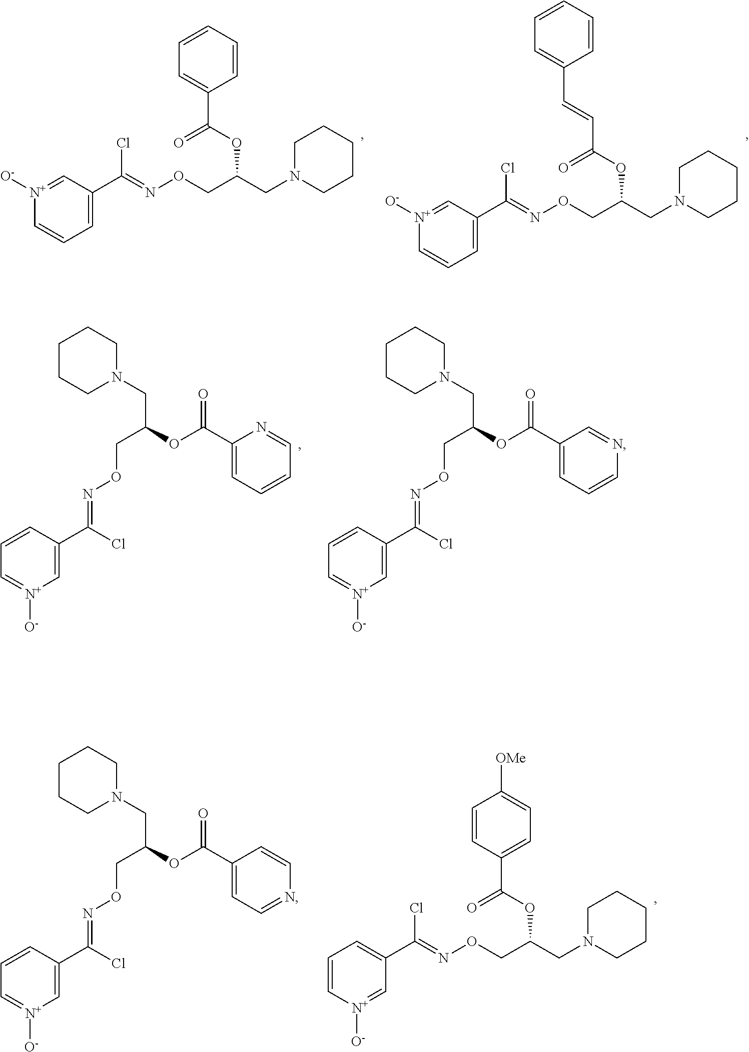

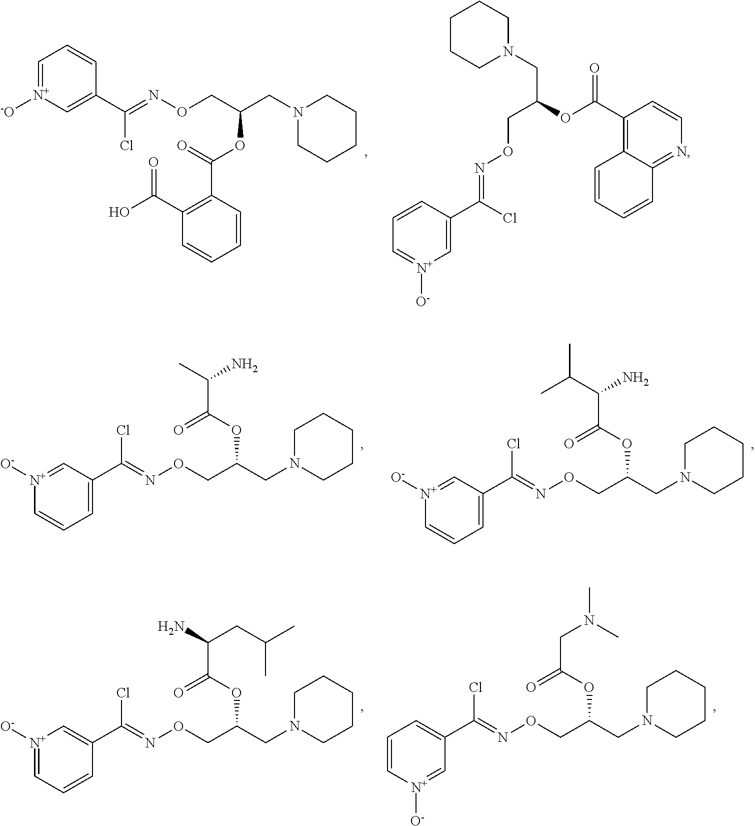

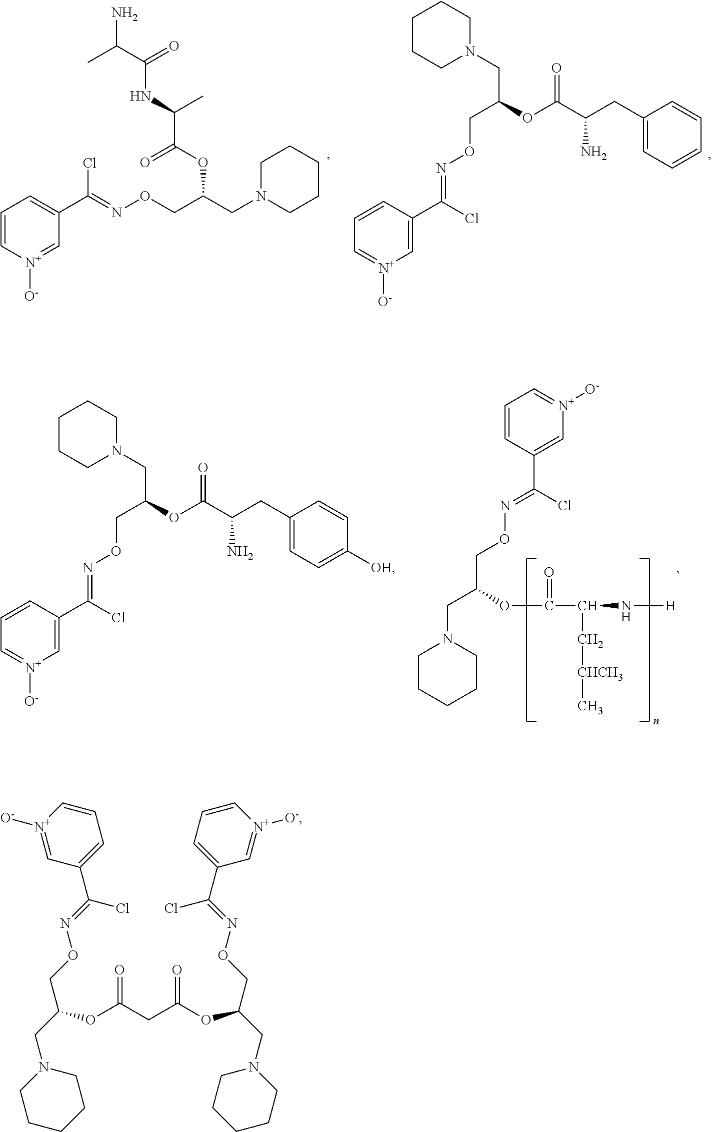

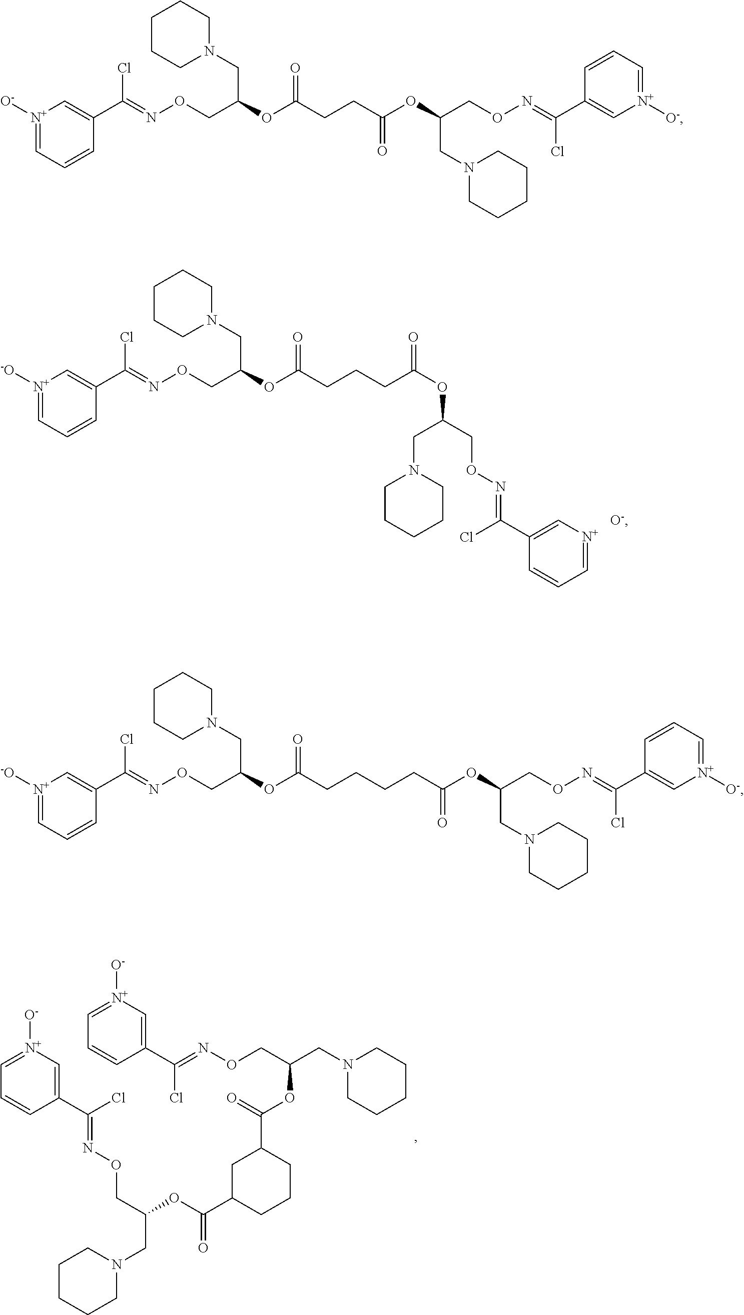

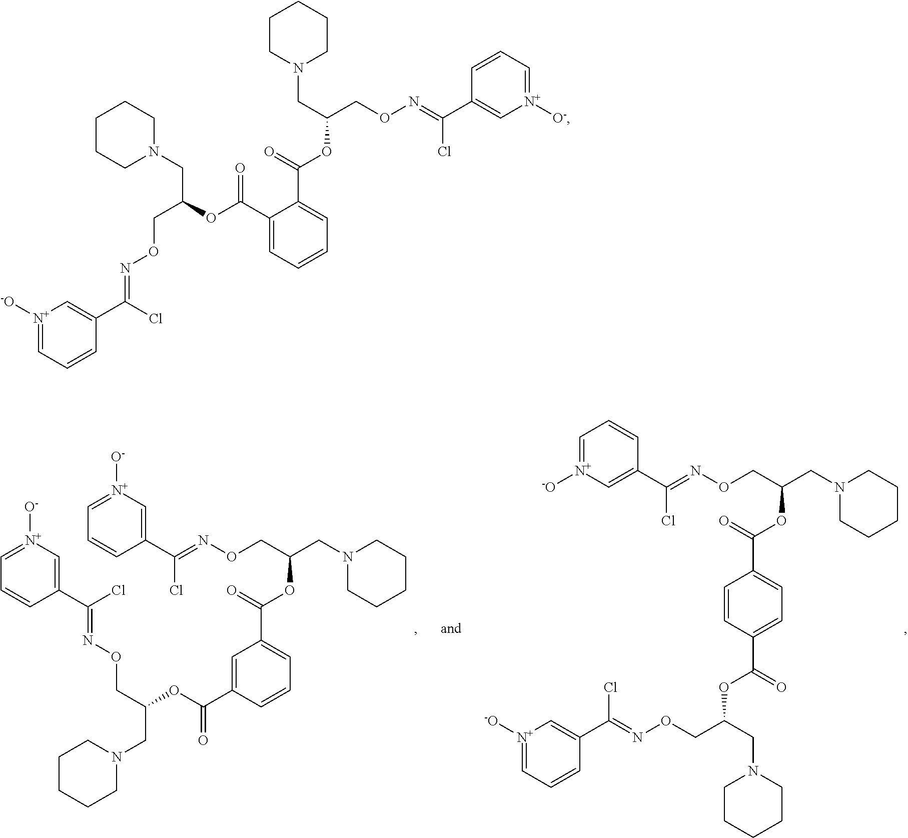

wherein n is 1-50;
or a pharmaceutically acceptable salt thereof.

28. The composition of 27, wherein the compound of Formula IC has a structure selected from:

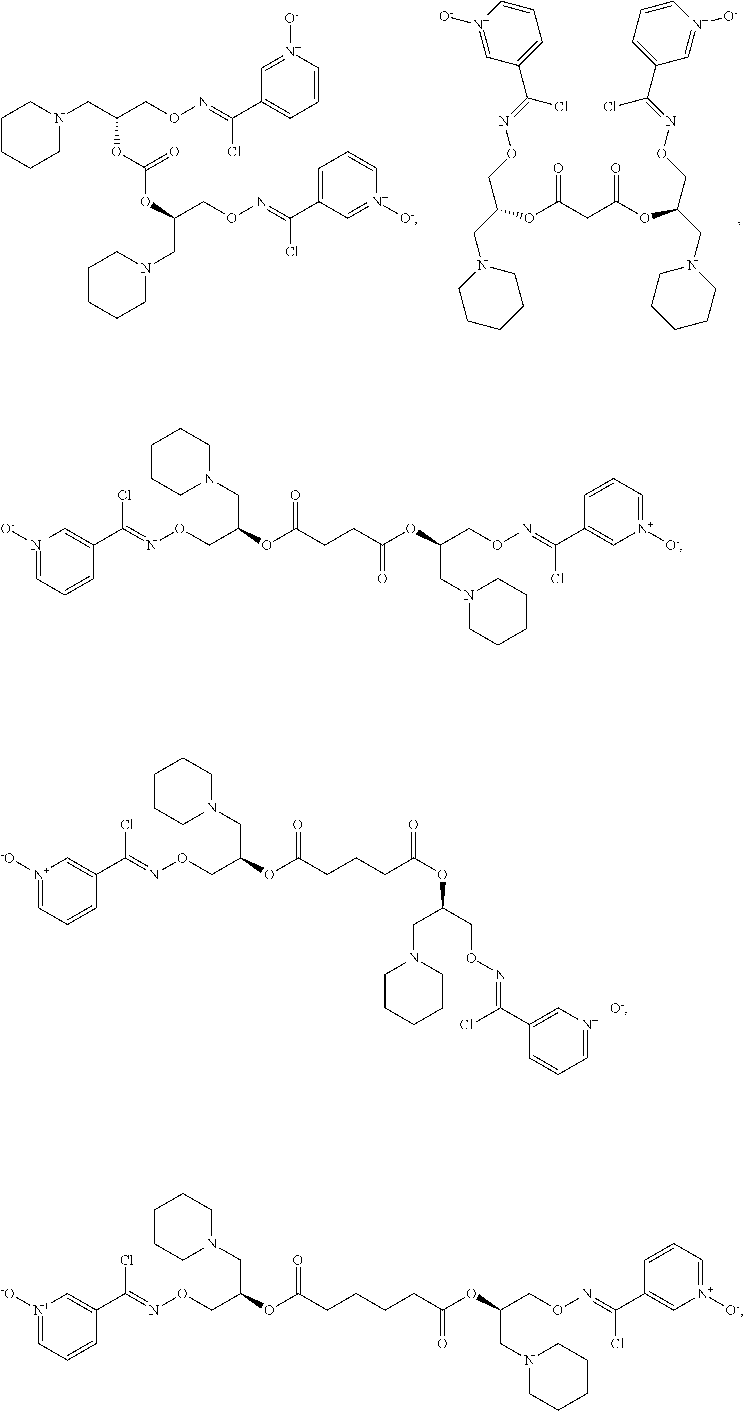

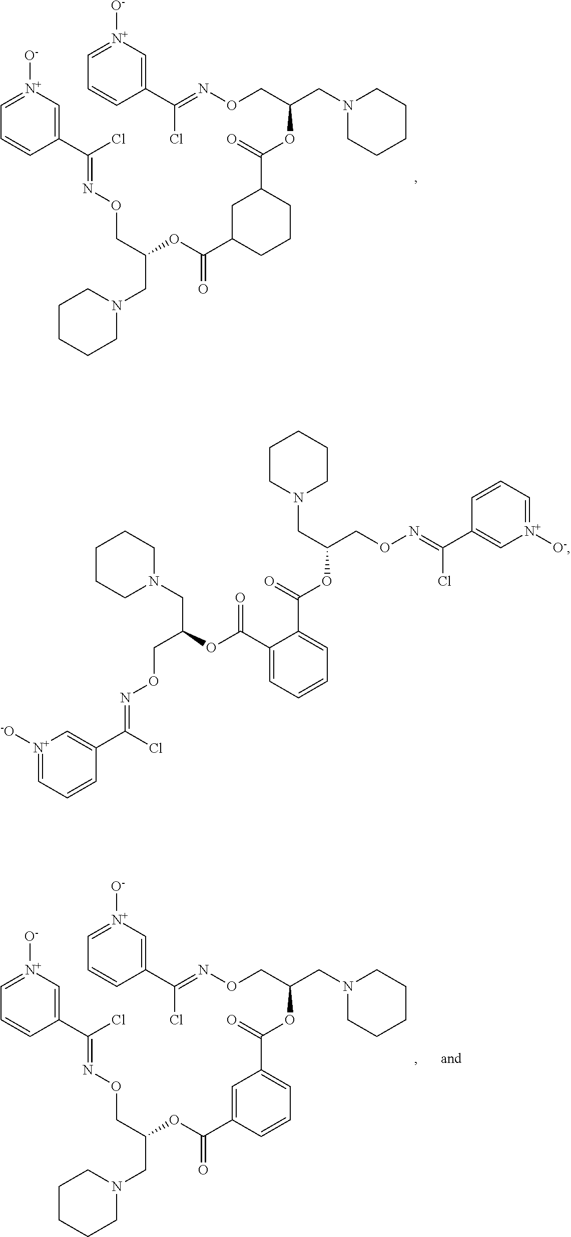

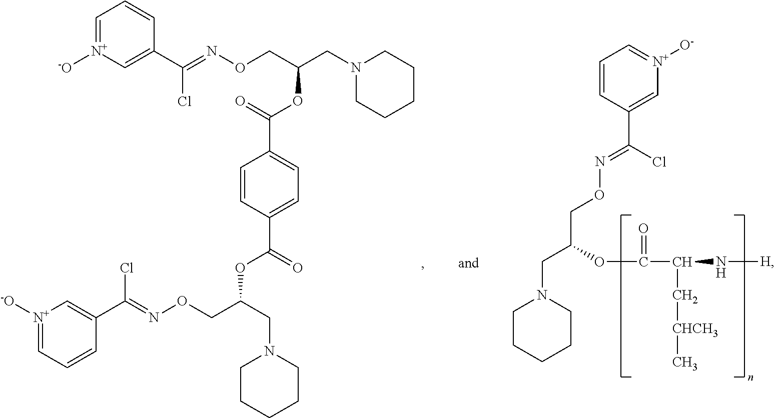

wherein n is 1-50
or a pharmaceutically acceptable salt thereof.

29. The composition of 21, wherein the compound of Formula I is a compound of Formula ID:

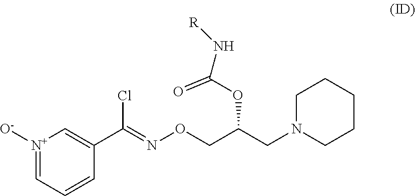

wherein R is selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

30. The composition of 29, wherein the compound of Formula ID is a compound having a structure selected from the group consisting of:

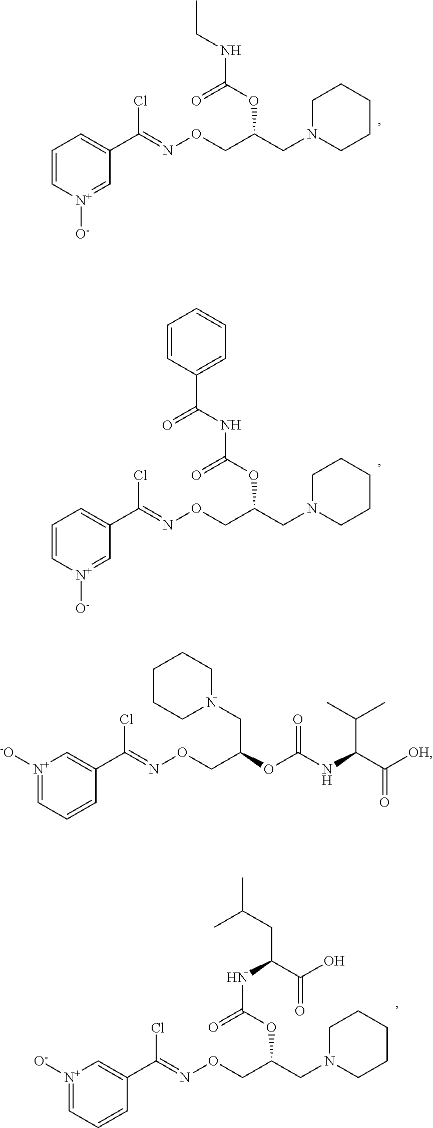

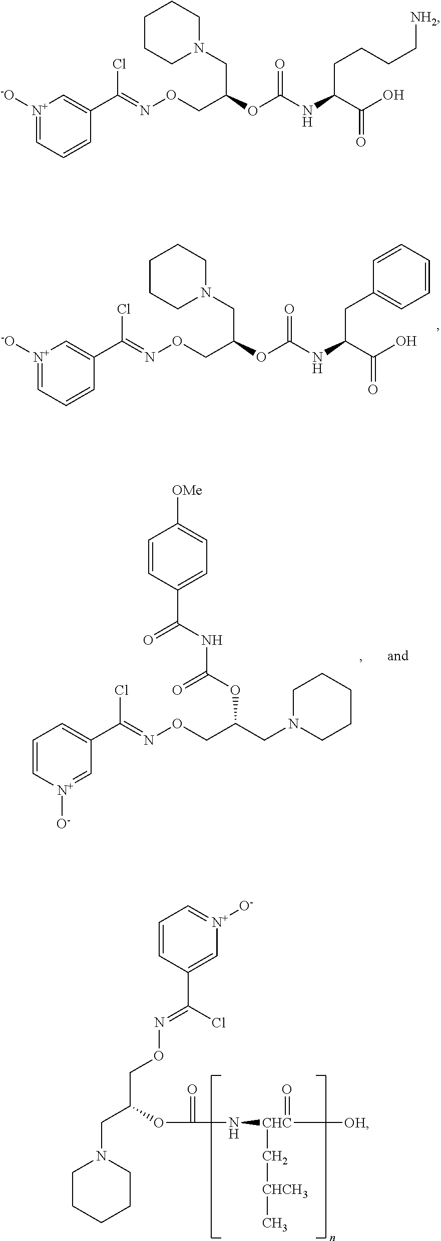

wherein n is 1-50;
or a pharmaceutically acceptable salt thereof.

31. A composition comprising:
a therapeutically effective amount of a compound of Formula II:

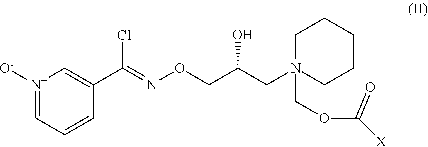

wherein X is selected from the group consisting of R, [O—R], [NR¹R²], and [NRH]
wherein O is oxygen, N is nitrogen, H is hydrogen, and
R, R¹ and R² are independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol
or a pharmaceutically acceptable salt thereof.

32. The composition of 31, wherein the compound of Formula II is selected from the group consisting of:

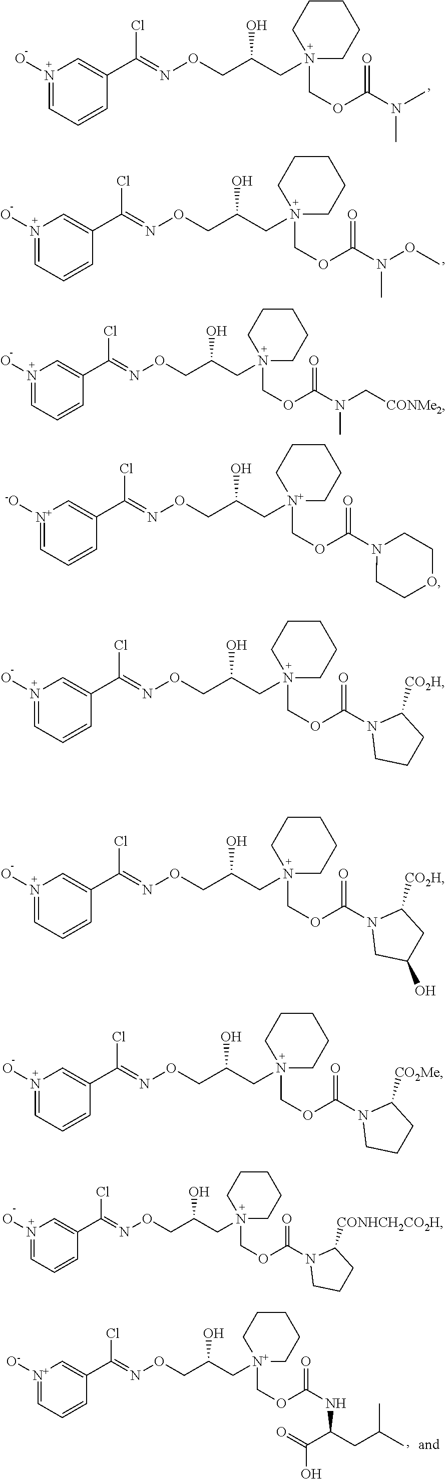

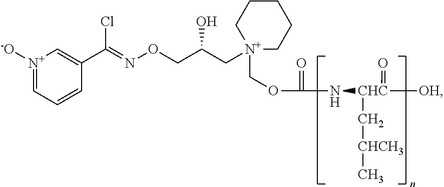

wherein n is 1-50;
or a pharmaceutically acceptable salt thereof

33. A composition comprising:
a therapeutically effective amount of a compound of Formula III:

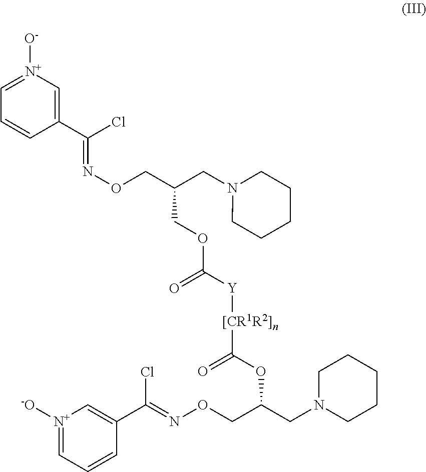

wherein X is selected from the group consisting of O, N, or absent,
Y is selected from the group consisting of O, N, or absent,
n is 1-10,
R¹ and R² are selected independently from each other and for each CR¹R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

34. A composition comprising:
a therapeutically effective amount of a compound of Formula IV:

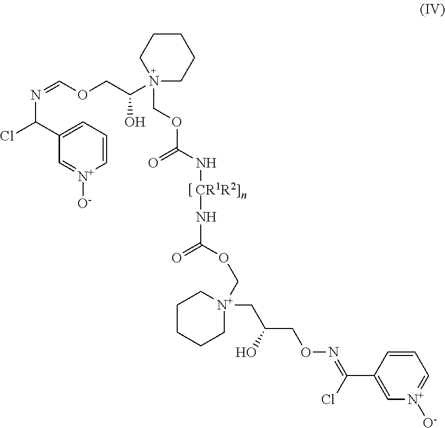

n is 1-10,
R¹ and R² are selected independently from each other and for each CR₁R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

35. A composition comprising:
a therapeutically effective amount of a compound of Formula V

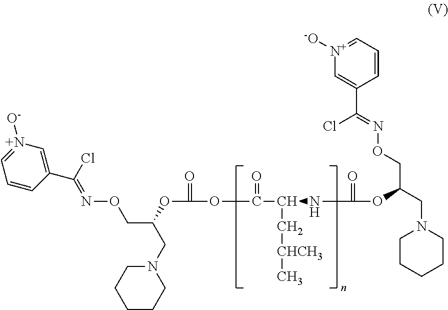

wherein n is 1-50,
or a pharmaceutically acceptable salt thereof.

36. A composition comprising:
a therapeutically effective amount of a compound of Formula VI:

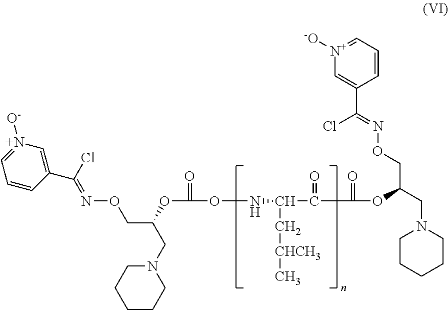

wherein n is 1-50;
or a pharmaceutically acceptable salt thereof.

37. A composition comprising:
a therapeutically effective amount of a compound of Formula VII:

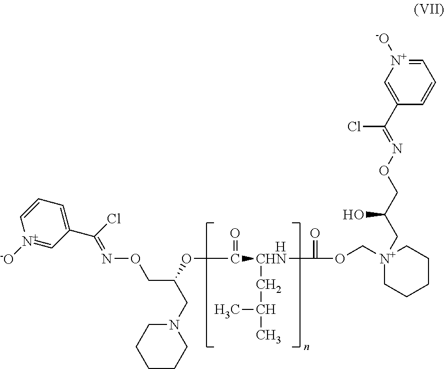

wherein n is 1-50,
or a pharmaceutically acceptable salt thereof.

38. A composition comprising:
a therapeutically effective amount of a compound of selected from the group consisting of:

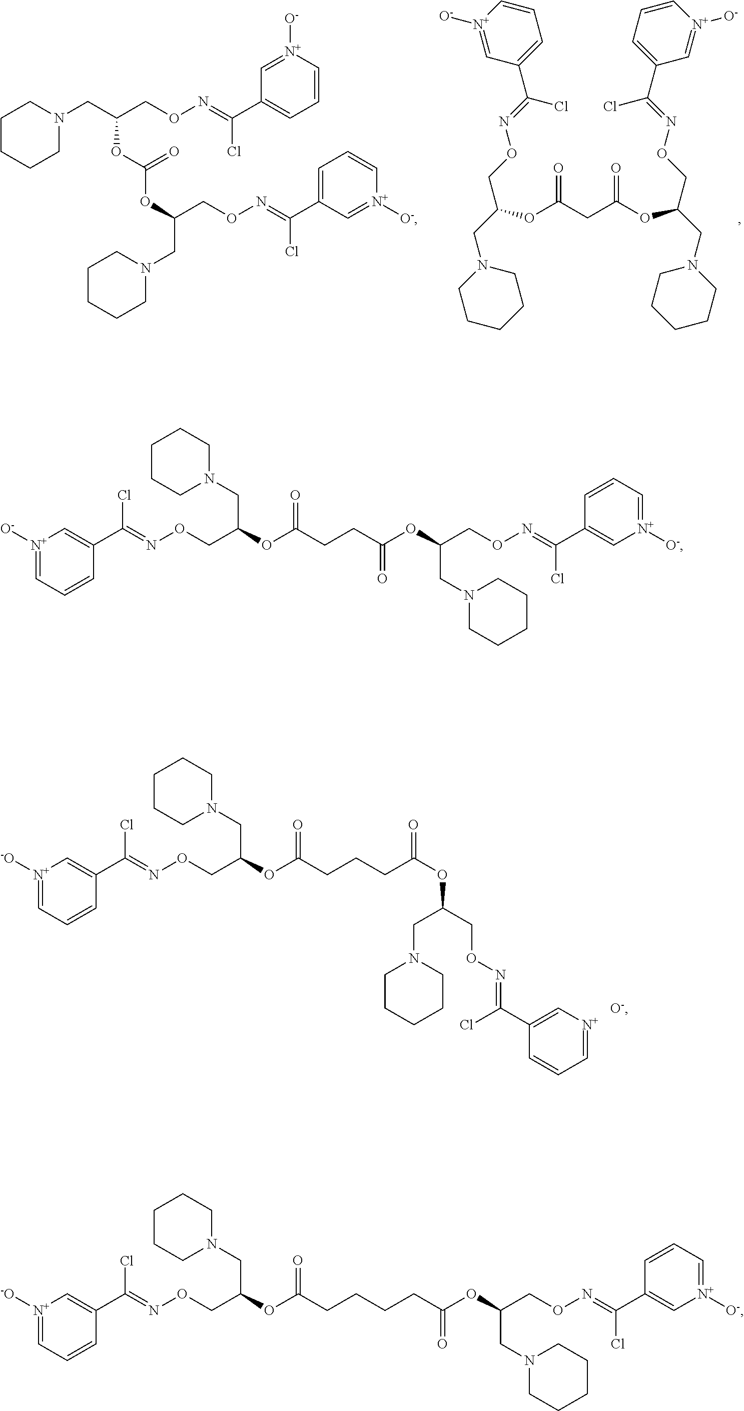

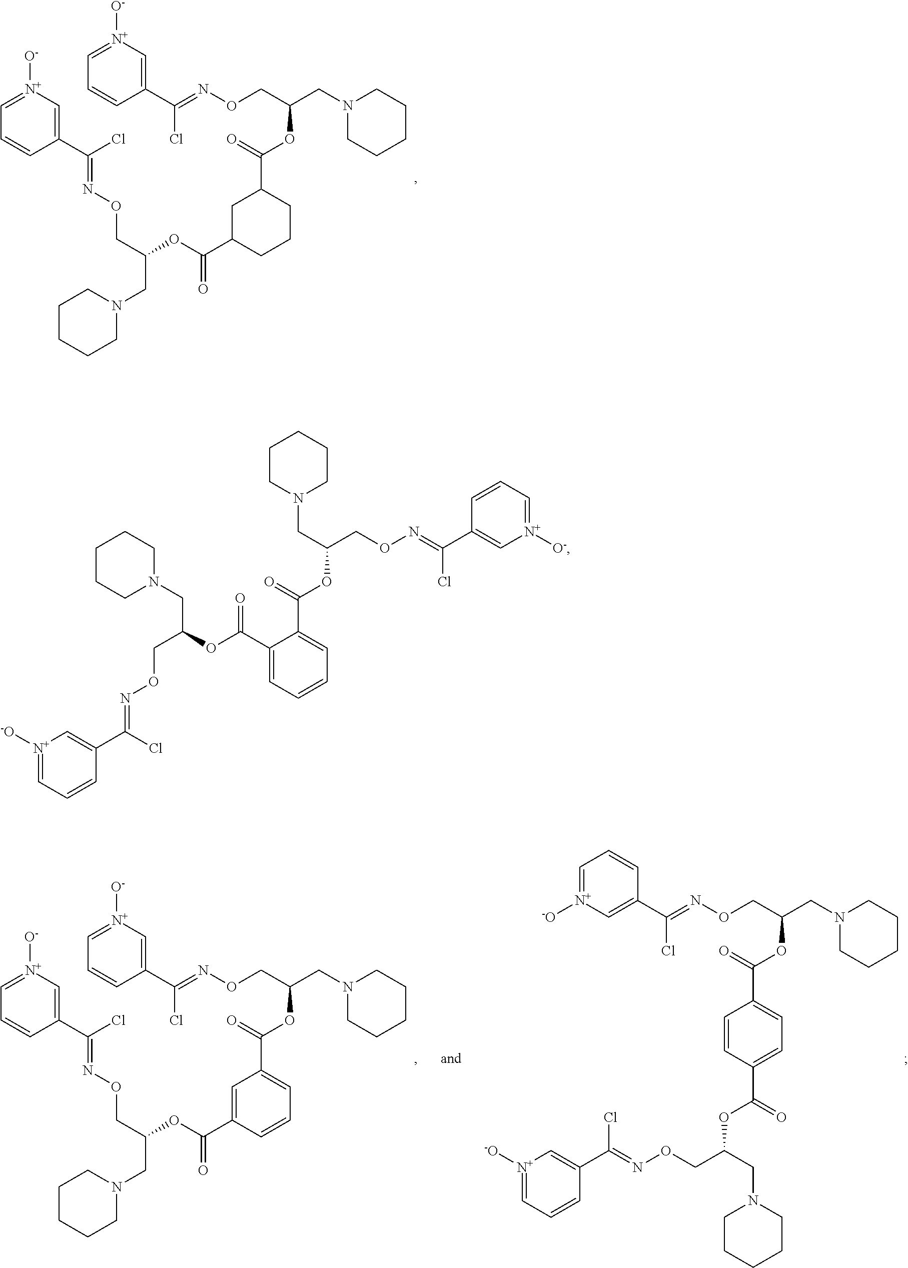

or a pharmaceutically acceptable salt thereof.

39. A composition comprising:
a therapeutically effective amount of a compound of selected from the group consisting of:

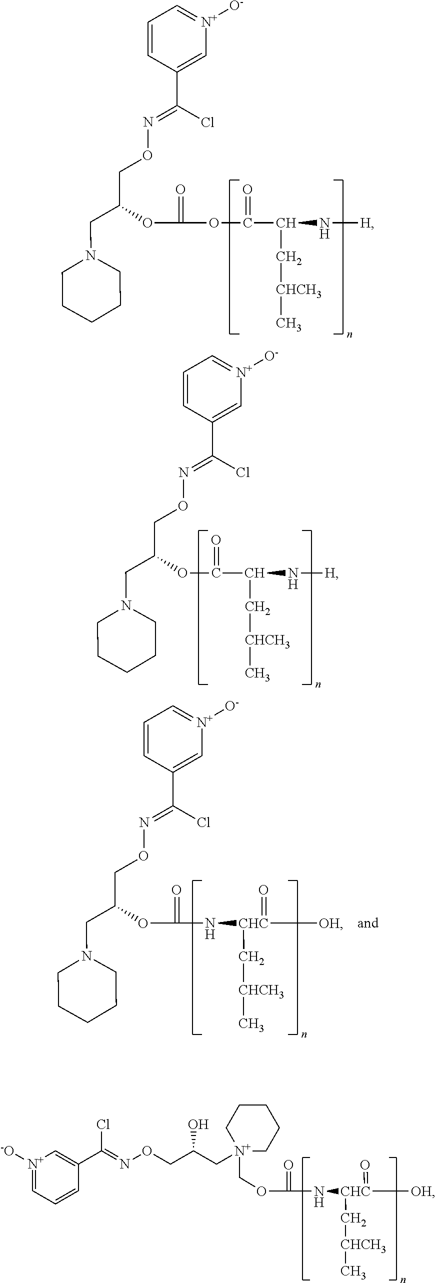

wherein n is 1-50;
or a pharmaceutically acceptable salt thereof.

40. The composition of 21-39, wherein the first pharmaceutically acceptable salt is a pharmaceutically acceptable salt of the compound of Formula I or Formula II is selected from: sulfate, citrate, acetate, oxalate, chloride, hydrochloride, bromide, hydrobromide, iodide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

41. The composition of 21-40, wherein the compound of Formula I is present as a stereoisomer and comprises a sub-structure selected from the group consisting of (+)-(R)—N-[2-hydroxy-3-(1-piperidinyl)-propoxy]-pyridine-1-oxide-3-carboximidoyl chloride, and (−)-(S)—N-[2-hydroxy-3-(1-piperidinyl)-propoxy]-pyridine-1-oxide-3-carboximidoyl chloride, (Z)—(R)—N-[2-hydroxy-3-(1-piperidinyl)-propoxy]-pyridine-1-oxide-3-carboximidoyl chloride, (E)-(R)—N-[2-hydroxy-3-(1 piperidinyl)-propoxy]-pyridine-1-oxide-3-carboximidoyl chloride, (Z)—(S)—N-[2-hydroxy-3-(1-piperidinyl)-propoxy]-pyridine-1-oxide-3-carboximidoyl chloride; and (E)-(S)—N-[2-hydroxy-3-(1-piperidinyl)-propoxy]-pyridine-1-oxide-3-carboximidoyl chloride.

42. The composition of 21-41, wherein the compound of Formula 1 or Formula II is present in a form selected from the group consisting of polymorphs, crystals, and combinations thereof.

43. The composition of 21-42, wherein the composition further comprises at least one excipient.

44. The composition of 43, wherein the at least one excipient is selected from the group consisting of antiadherents, binders, coatings, disintegrants, gel forming agents, fillers, flavors and colors, glidants, lubricants, preservatives, sorbents and sweeteners.

45. The composition of 21-44, wherein the at least one additional active pharmaceutical ingredient is selected from the group consisting of bimoclomol, miglustat, eliglustat, N-acetyl-L-leucine and 2-hydroxypropyl-β-cyclodextrin, and combinations thereof.

46. The composition of 45, wherein the at least one additional active pharmaceutical ingredient is selected from arimoclomol, miglustat, N-acetyl-L-leucine, and combinations thereof.

47. The composition of 46, wherein the at least one additional active pharmaceutical ingredient is a combination of arimoclomol, miglustat and N-acetyl-L-leucine.

48. The composition of 21-47, wherein the composition is formulated for oral administration.

49. The composition of 48, wherein the composition is a solid oral dosage formulation.

50. The composition of 49, wherein the solid oral dosage formulation is selected from the group consisting of a sublingual, a gummy, a chewable tablet, a rapidly dissolving tablet, a tablet, a capsule, a caplet, a troche, a lozenge, an oral powder, a solution, a thin strip, an oral thin film (OTF), an oral strip, and a rectal film.

51. The composition of 21-48, wherein the composition is a liquid oral dosage formulation.

52. The composition of 51, wherein the liquid oral dosage formulation is selected from the group consisting of syrups, emulsions, solutions, and suspensions.

53. The composition of 21-52, wherein the therapeutically effective amount of the compound of Formula I to Formula VII ranges from about 1 to about 5000 mg per day.

54. The composition of 53, wherein the therapeutically effective amount of the compound of Formula I to Formula VII is administered as a unit dose.

55. A composition comprising:
a therapeutically effective amount of a compound of Formula I:

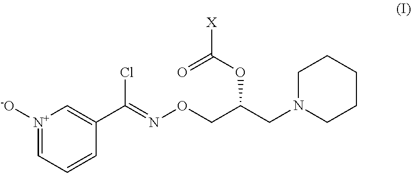

wherein X is selected from the group consisting of R, [O—R], [NR¹R²], and [NRH], where O is oxygen, N is nitrogen, and H is hydrogen;
R, R¹ and R² are independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof;
and an extended-release agent.

56. The composition of 55, wherein the compound is a compound having a structure of Formula IA:

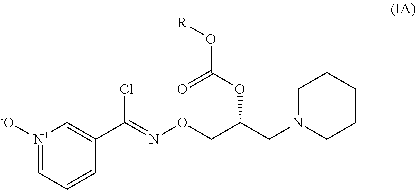

wherein R is selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

57. The composition of 56, wherein the compound of Formula IA is a compound having a structure selected from the group consisting of:

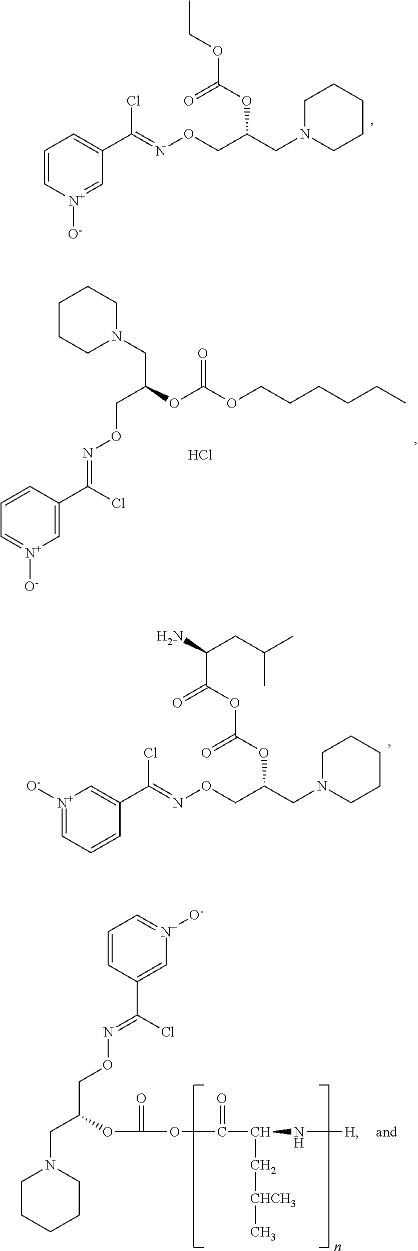

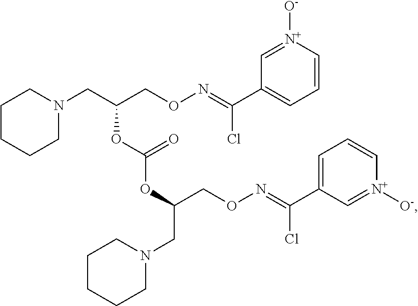

wherein n is 1-50
or a pharmaceutically acceptable salt thereof.

58. The composition of 57, wherein the compound of Formula I is a compound having a structure of Formula IB:

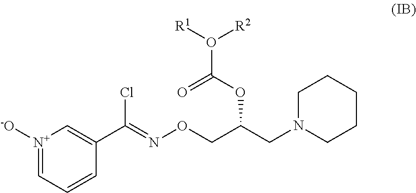

wherein R¹ and R² are independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

59. The composition of 58, wherein the compound of Formula IB is a compound having a structure selected from the group consisting of:

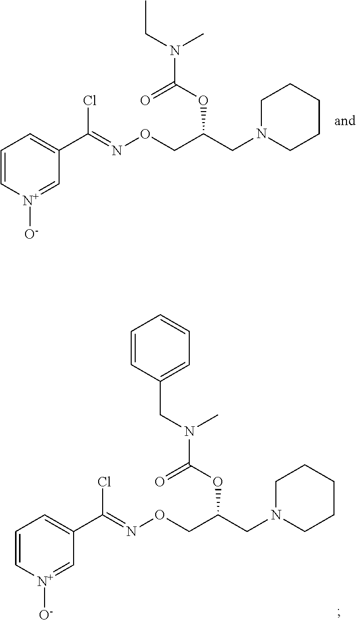 [SMILES: CCN(C)C(=O)O[C@@H](CO/N=C(\Cl)c1ccc[n+]([O-])c1)CN1CCCCC1 ; CN(Cc1ccccc1)C(=O)O[C@@H](CO/N=C(\Cl)c1ccc[n+]([O-])c1)CN1CCCCC1]

or a pharmaceutically acceptable salt thereof.

60. The composition of claim 55, wherein the compound of Formula I is a compound of Formula IC:

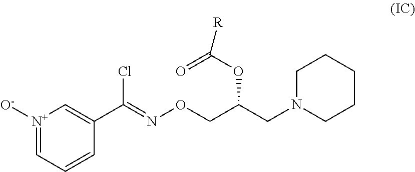

wherein R is independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

61. The composition of 60, wherein the compound of Formula IC is a compound having a structure selected from the group consisting of:

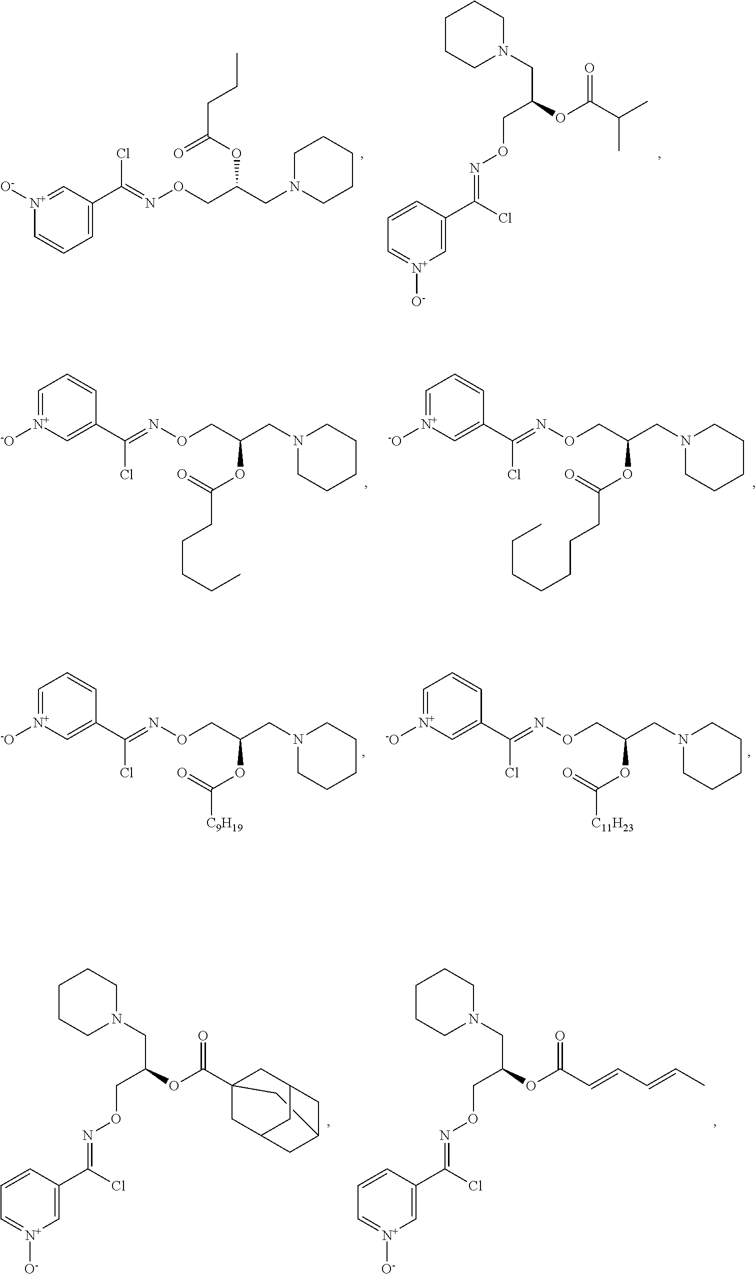

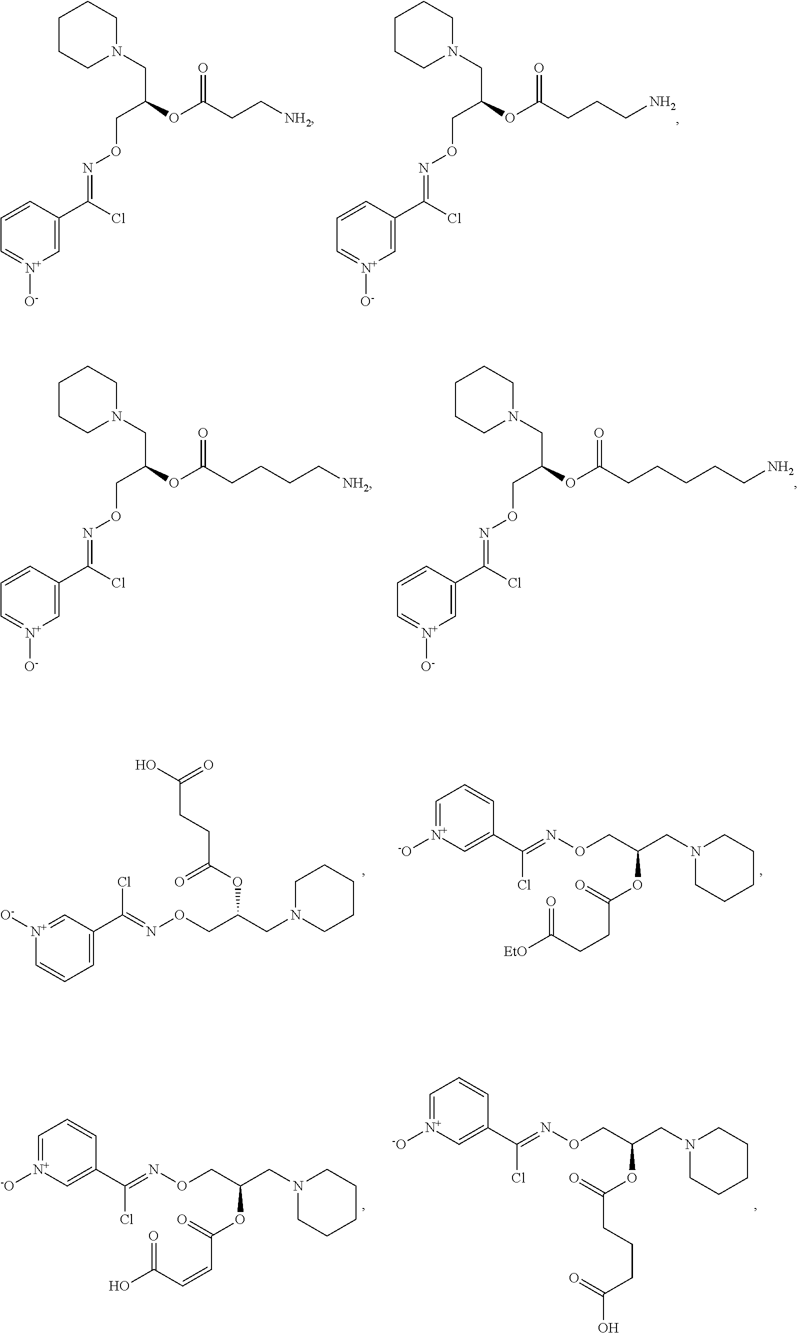

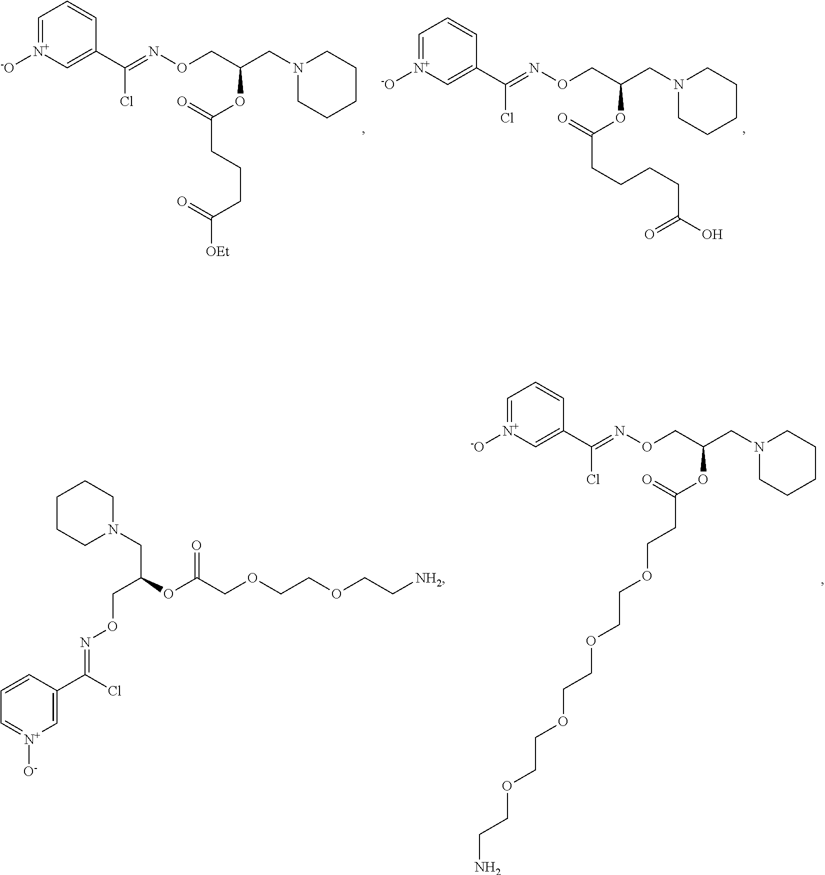

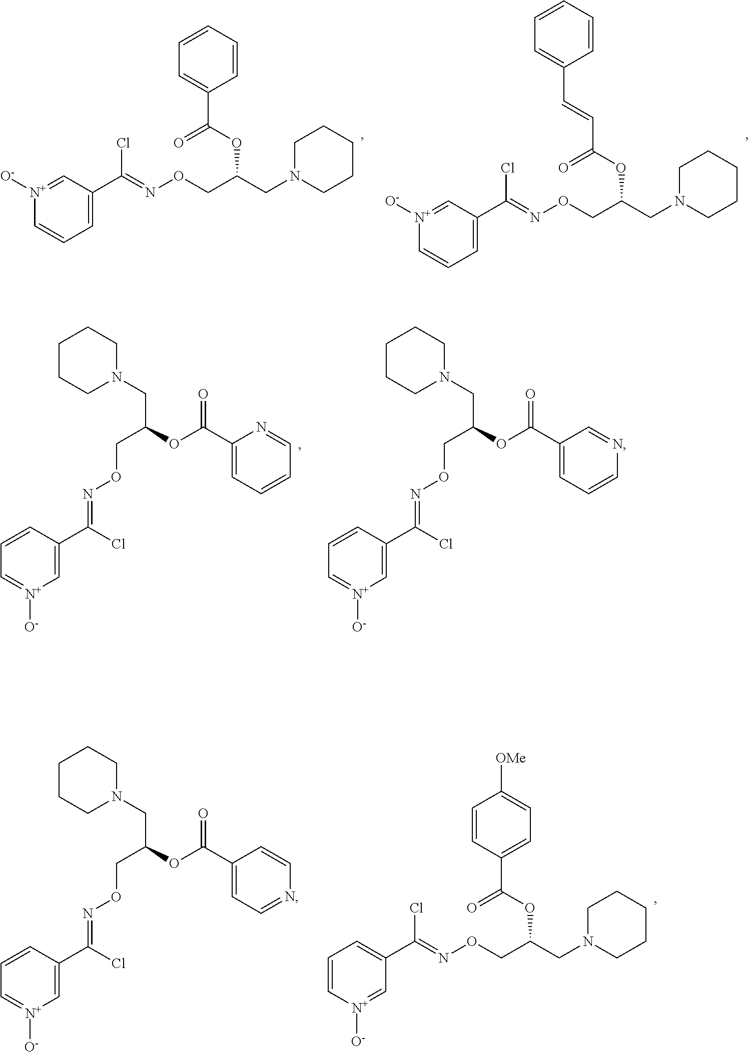

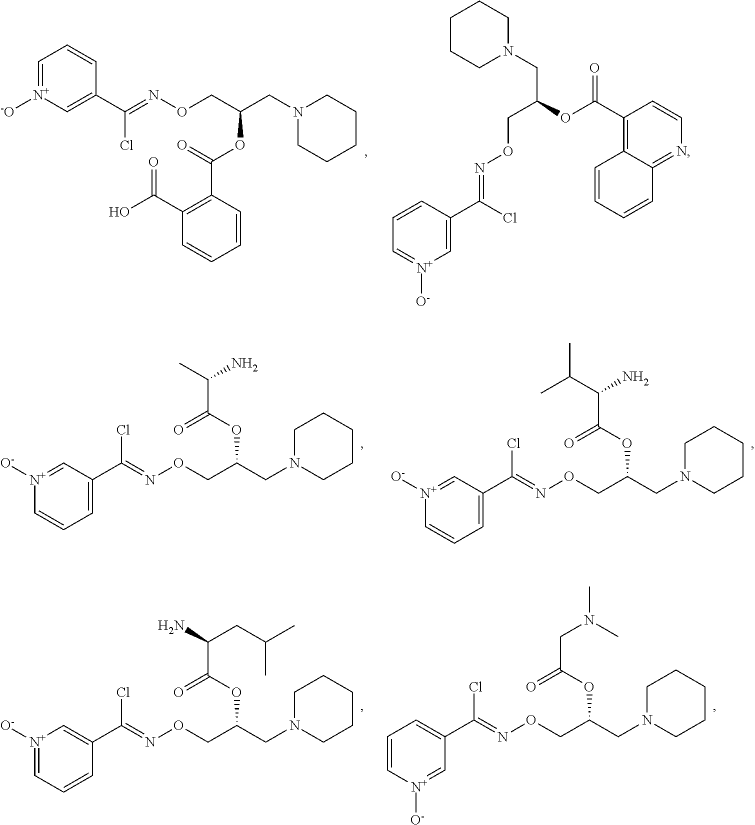

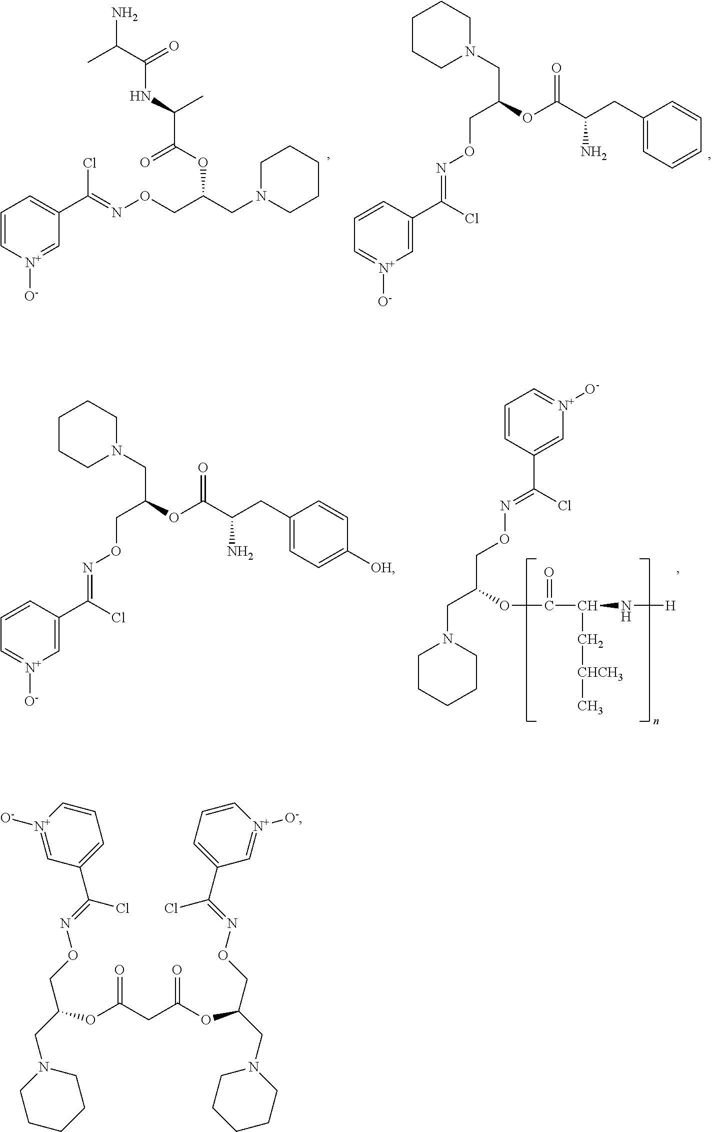

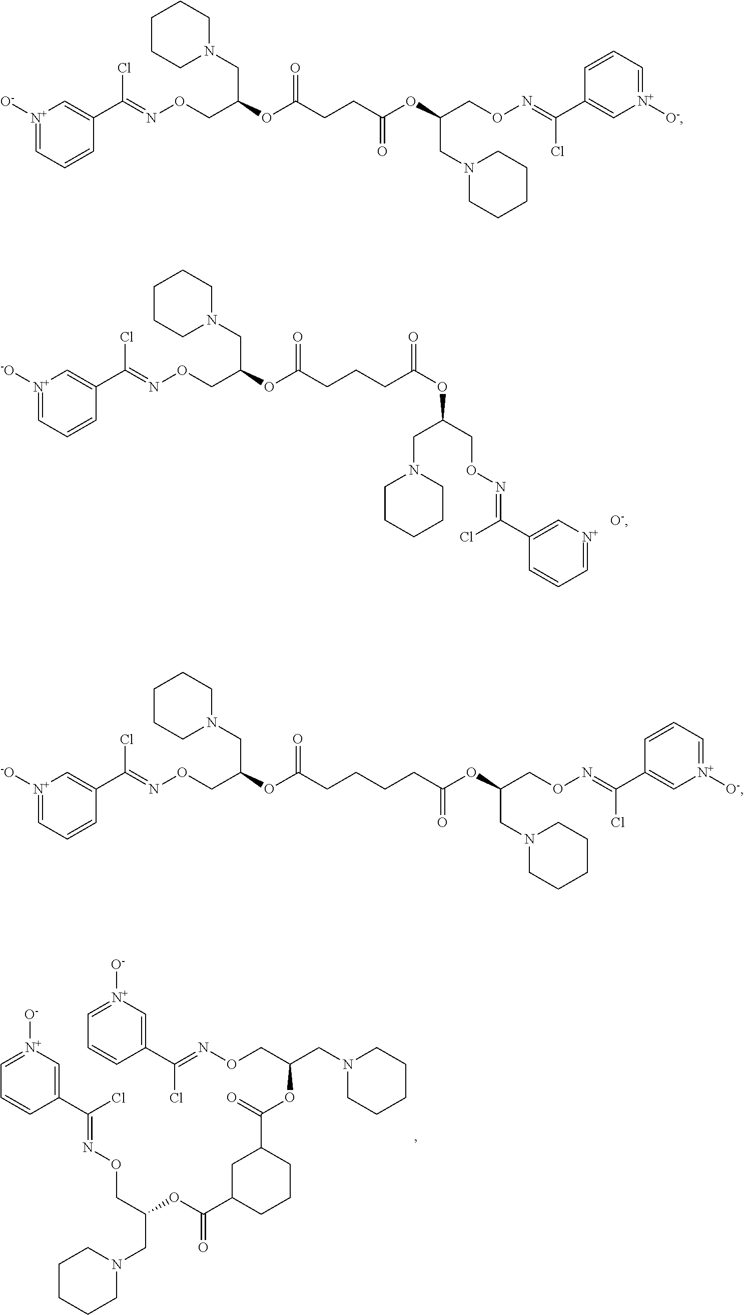

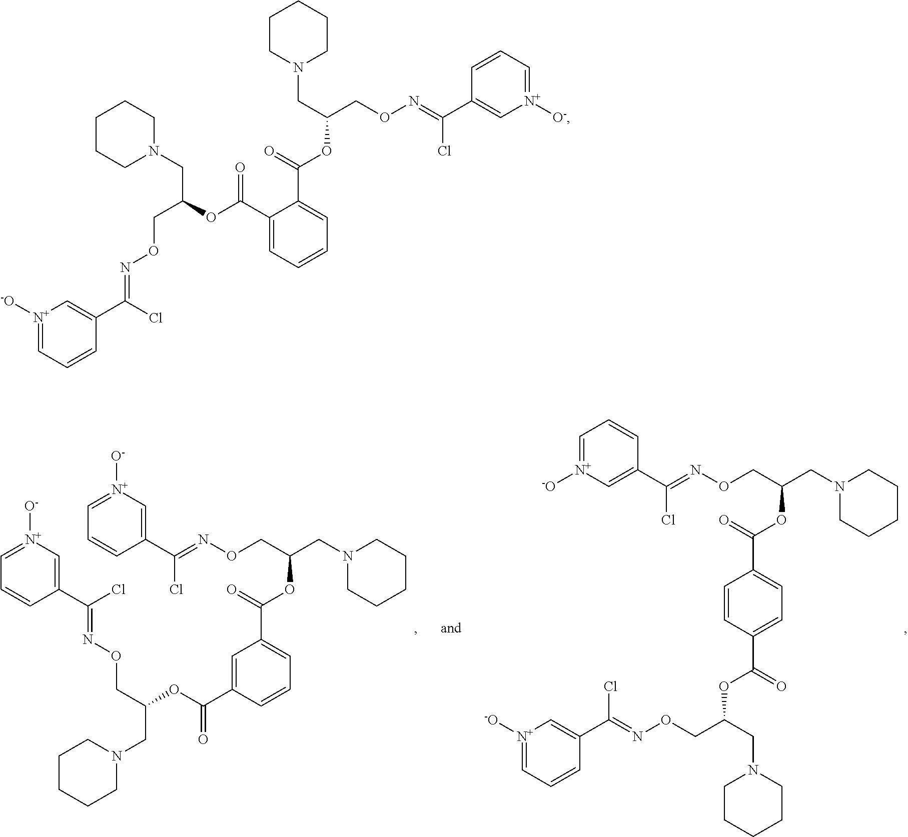

wherein n is 1-50;
or a pharmaceutically acceptable salt thereof.

62. The composition of 61, wherein the compound of Formula IC is a compound having a structure selected from the group consisting of:

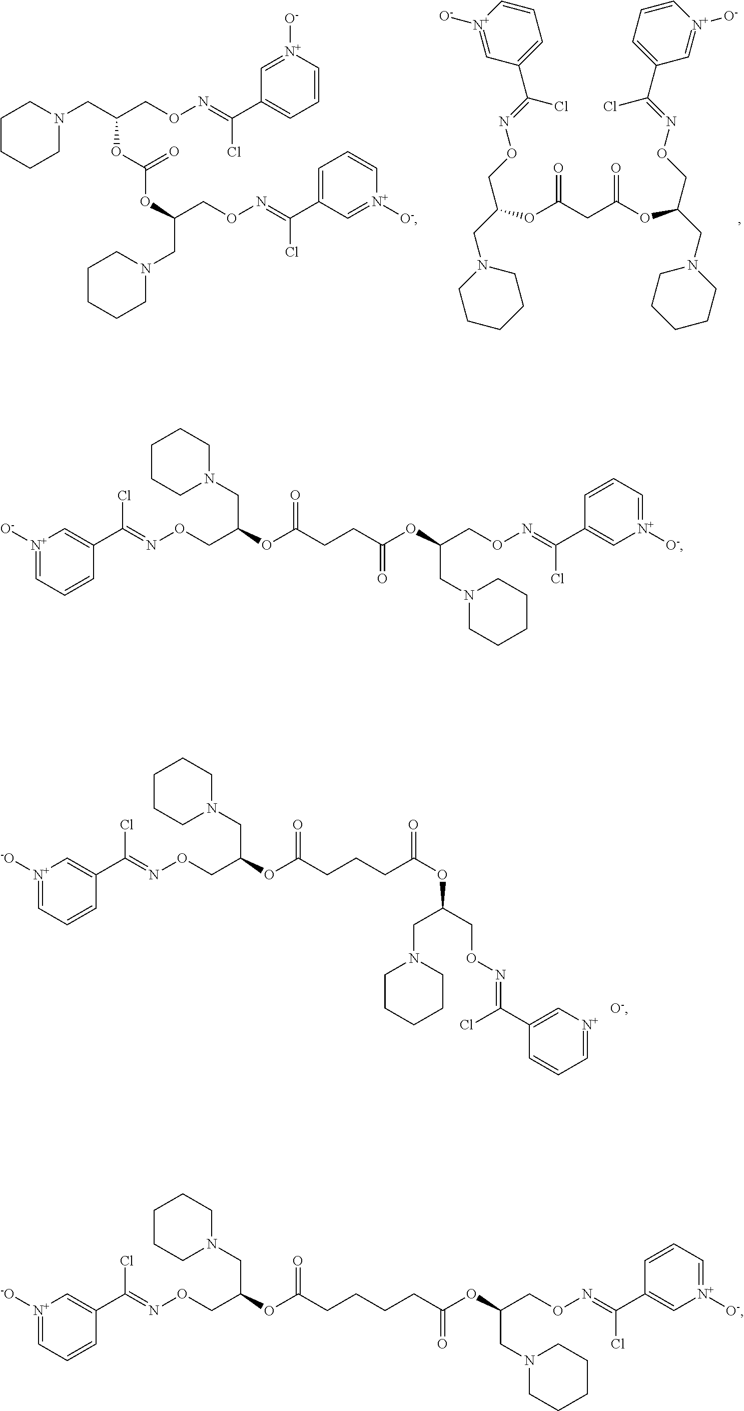

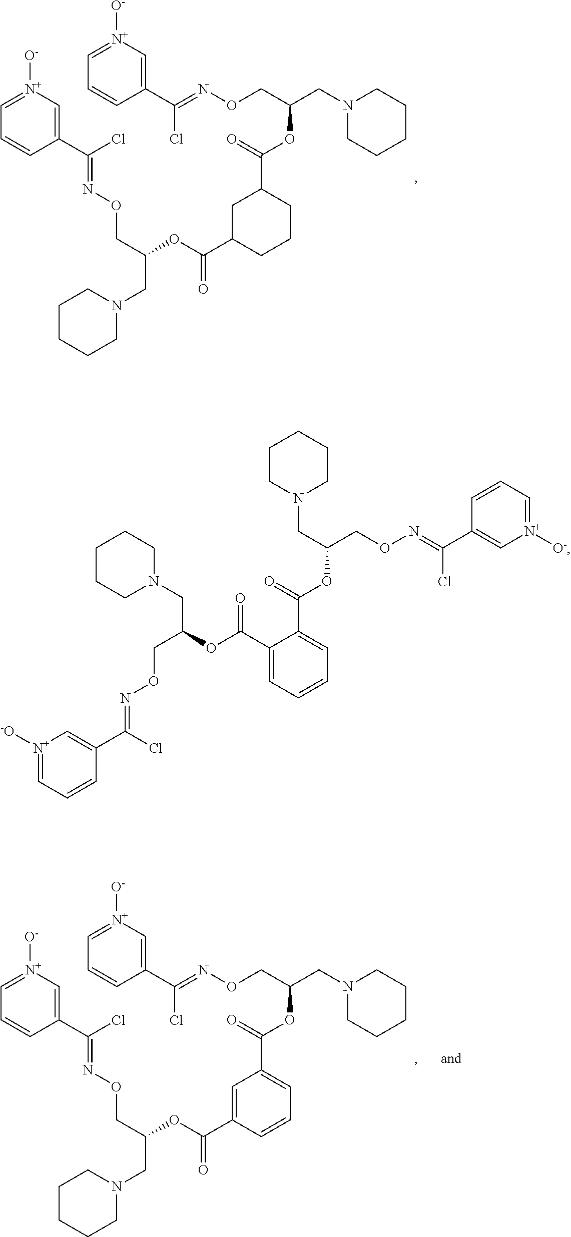

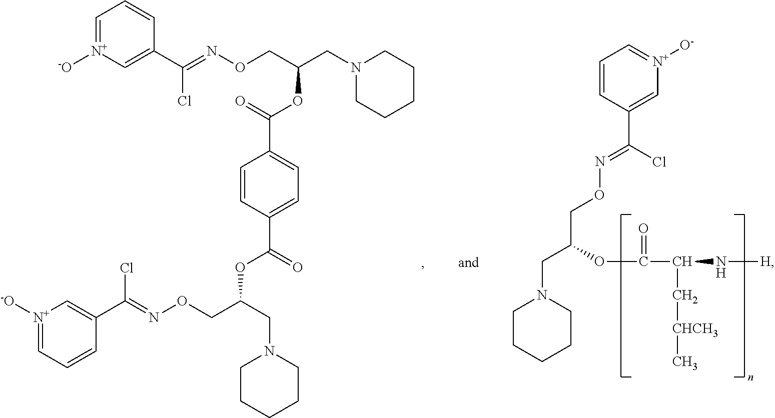

wherein n is 1-50;
or a pharmaceutically acceptable salt thereof.

63. The composition of 55, wherein the compound of Formula I is a compound having a structure of Formula ID:

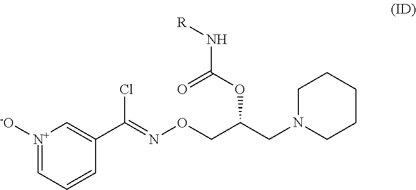

wherein R is selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

64. The composition of 63, wherein the compound of Formula ID is a compound having a structure selected from the group consisting of:

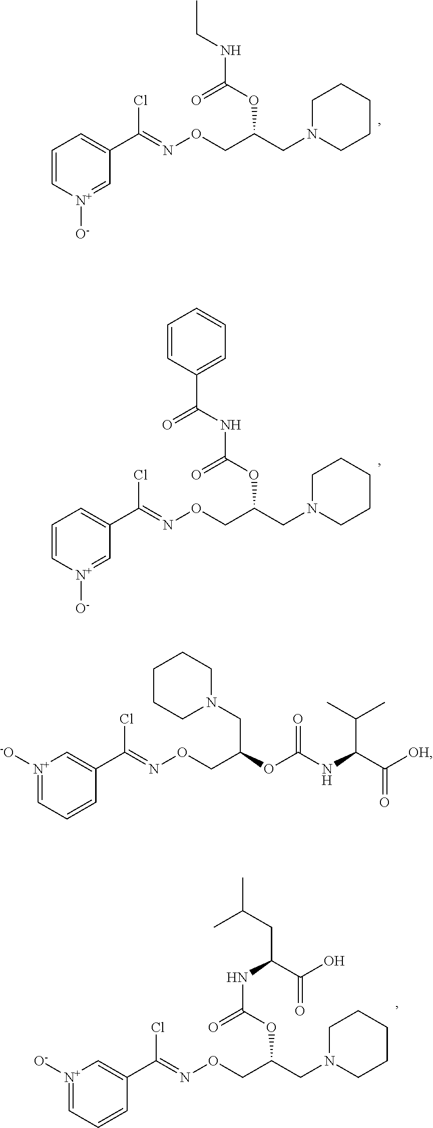

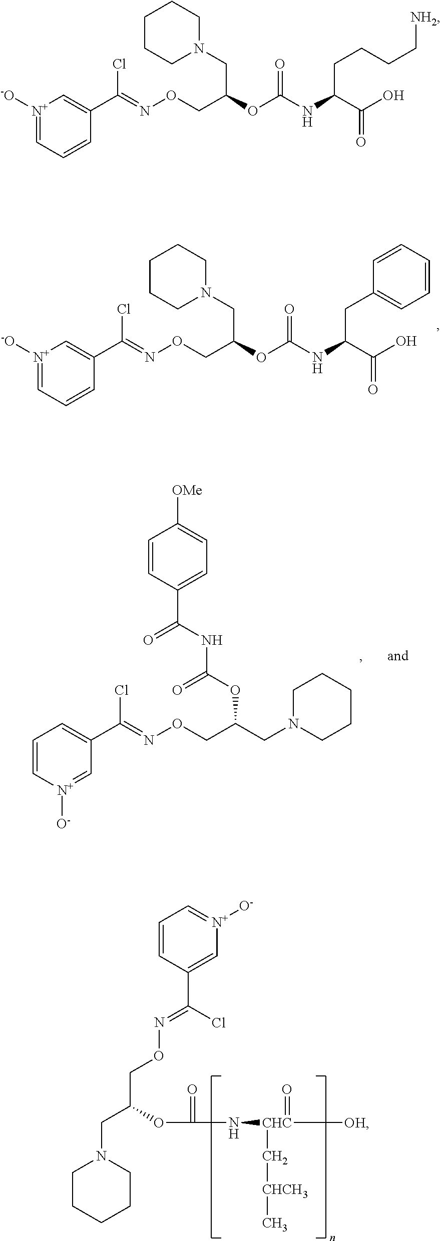

wherein n is 1-50;
or a pharmaceutically acceptable salt thereof.

65. A composition comprising:
a therapeutically effective amount of a compound of Formula II:

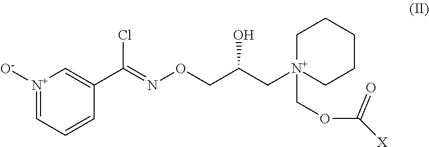

wherein X is selected from the group consisting of R, [O—R], [NR¹R²], and [NRH]
wherein O is oxygen, N is nitrogen, H is hydrogen, and
R, R¹ and R² are independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol
or a pharmaceutically acceptable salt thereof.

66. The composition of 65, wherein the compound of Formula II is selected from the group consisting of:

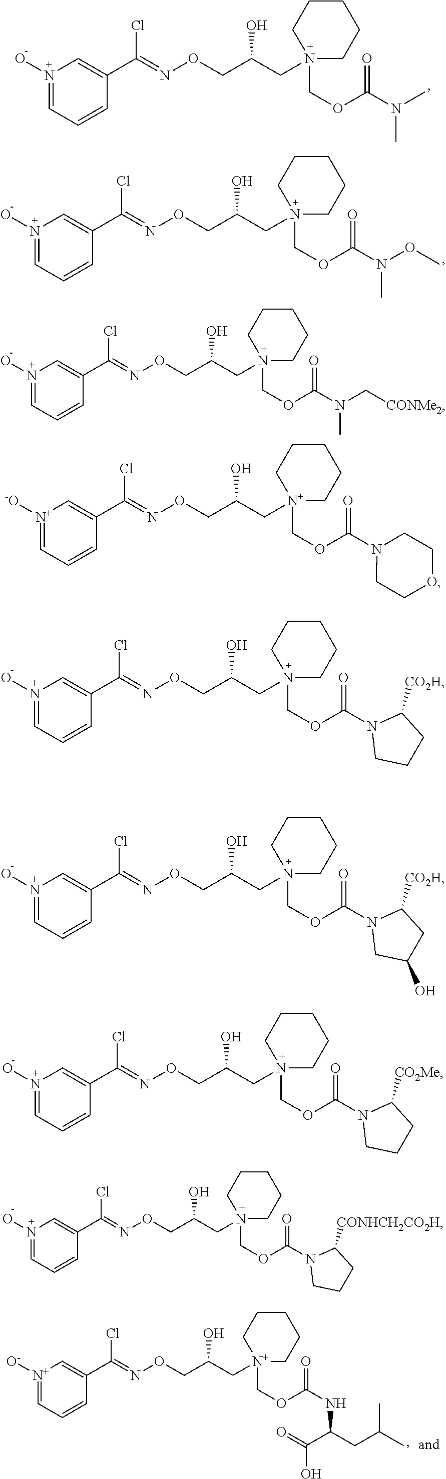

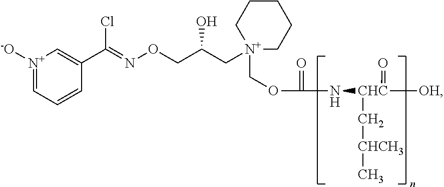

wherein n is 1-50;
or a pharmaceutically acceptable salt thereof

67. A composition comprising:
a therapeutically effective amount of a compound having a structure of Formula III:

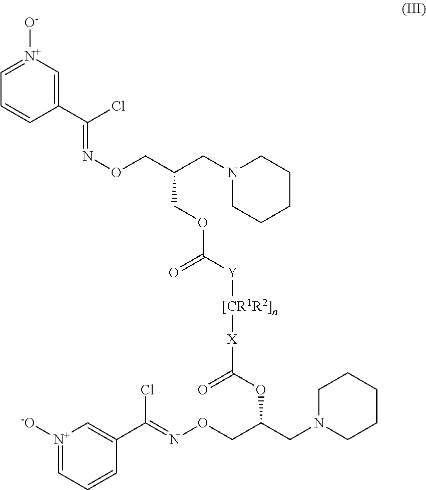

wherein X is selected from the group consisting of O, N, or absent,
Y is selected from the group consisting of O, N, or absent,
n is 1-10,
R¹ and R² are selected independently from each other and for each CR¹R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof; and
a release controlling agent.

68. A composition comprising:
a therapeutically effective amount of a compound having a structure of Formula IV:

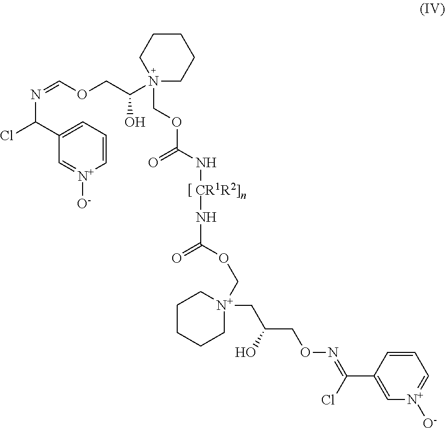

n is 1-10,
R¹ and R² are selected independently from each other and for each CR₁R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof; and
a release controlling agent.

69. A composition comprising:
a therapeutically effective amount of a compound having a structure of Formula V

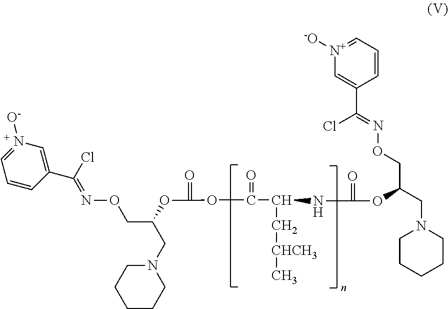

wherein n is 1-50,
or a pharmaceutically acceptable salt thereof; and
a release controlling agent.

70. A composition comprising:
a therapeutically effective amount of a compound having a structure of Formula VI:

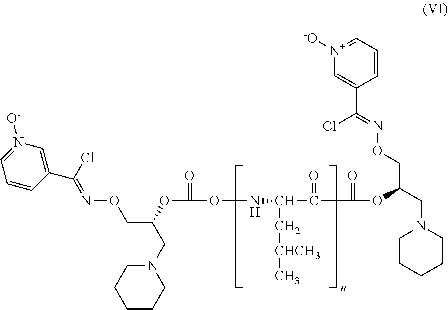

wherein n is 1-50;
or a pharmaceutically acceptable salt thereof; and
a release controlling agent.

71. A composition comprising:
a therapeutically effective amount of a compound having a structure of Formula VII:

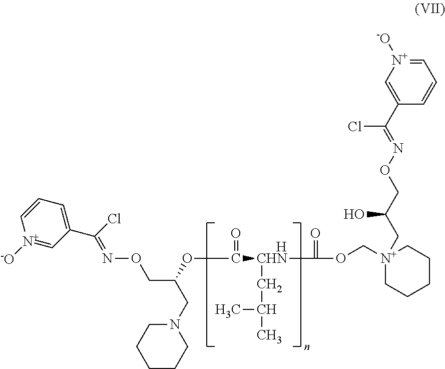

wherein n is 1-50,
or a pharmaceutically acceptable salt thereof; and
a release controlling agent.

72. A composition comprising:
a therapeutically effective amount of a compound having a structure selected from the group consisting of:

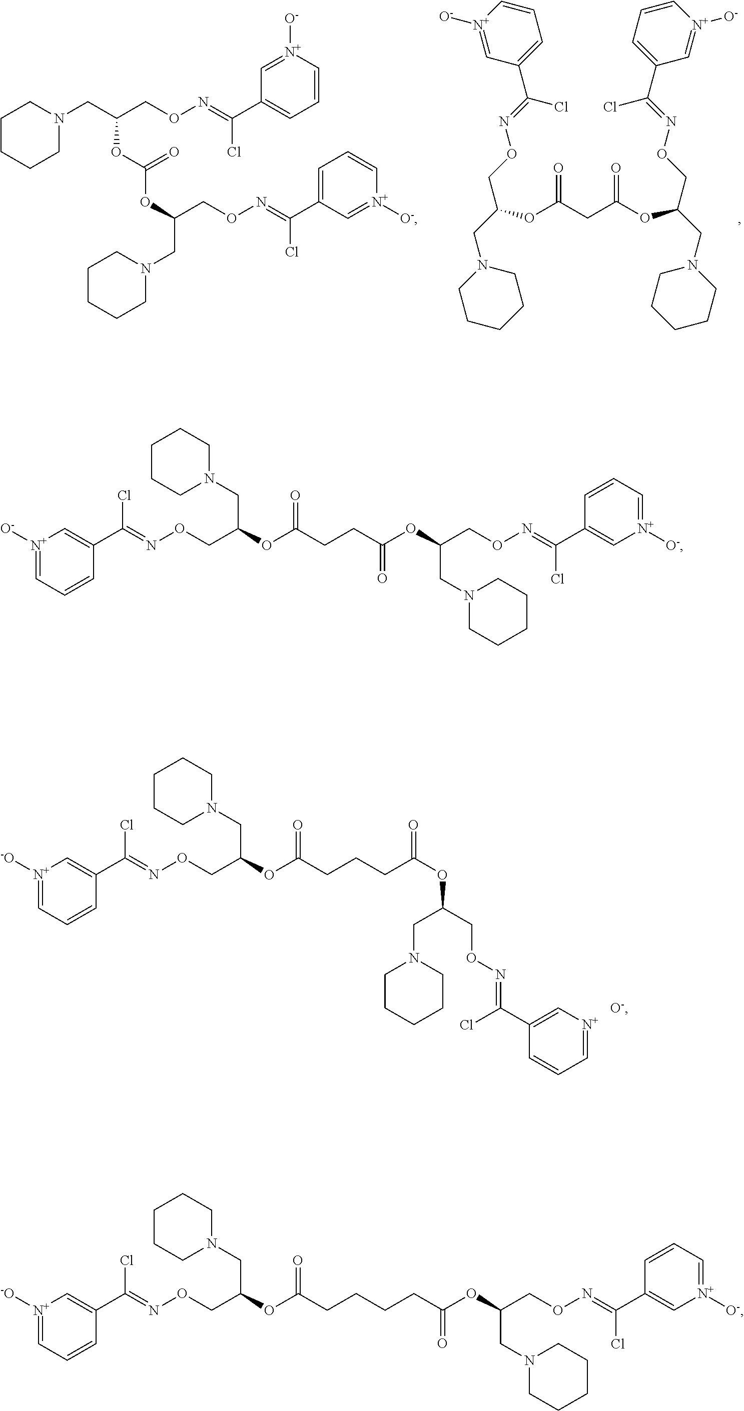

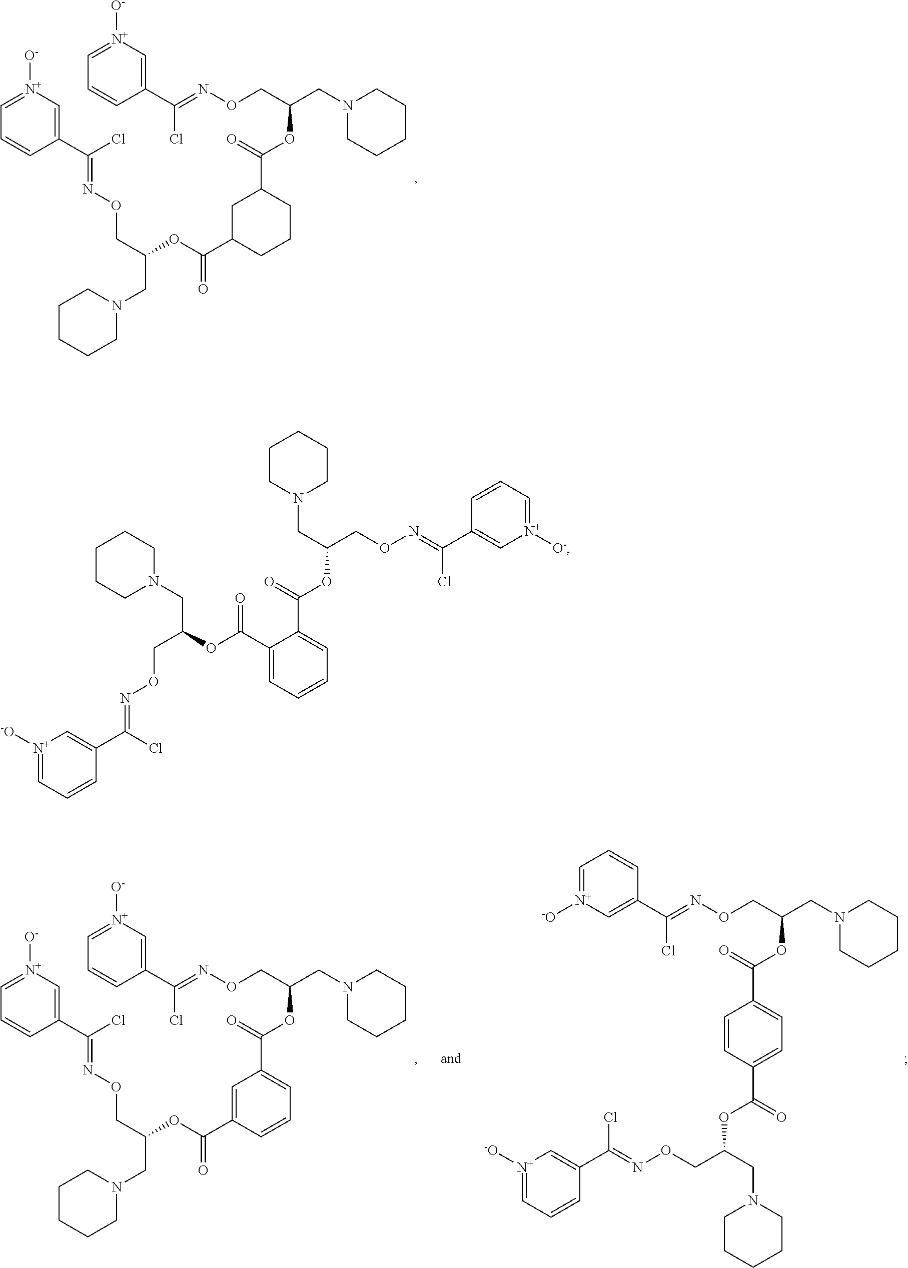

or a pharmaceutically acceptable salt thereof; and
a release controlling agent.

73. A composition comprising:
a therapeutically effective amount of a compound having a structure selected from the group consisting of:

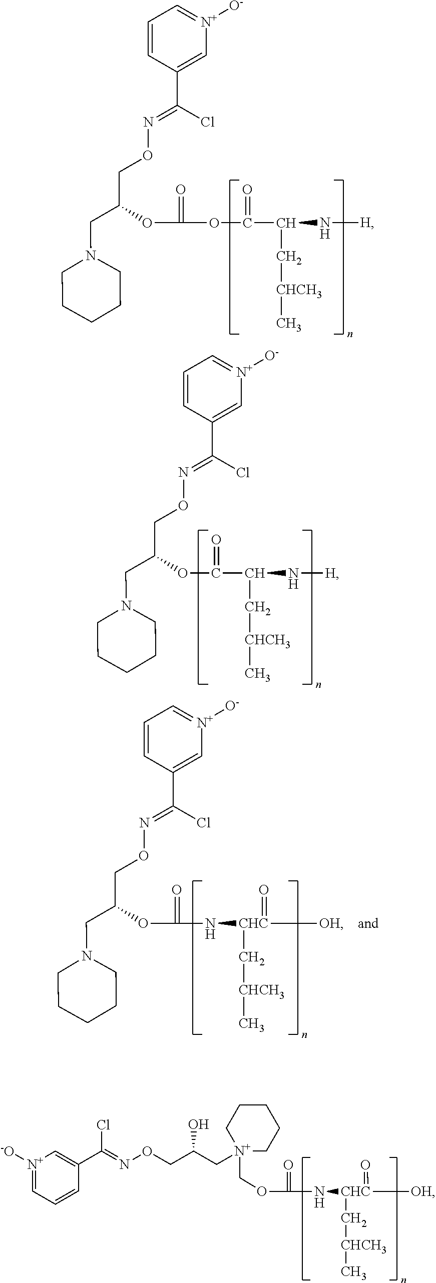

wherein n is 1-50;
or a pharmaceutically acceptable salt thereof; and
a release controlling agent.

74. The composition of 55-73, wherein the pharmaceutically acceptable salt is selected from: sulfate, citrate, acetate, oxalate, chloride, hydrochloride, bromide, hydrobromide, iodide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

75. The composition of 55-74, wherein the compound of Formula I to Formula VII is present as a stereoisomer and comprises a sub-structure selected from the group consisting of (+)-(R)—N-[2-hydroxy-3-(1-piperidinyl)-propoxy]-pyridine-1-oxide-3-carboximidoyl chloride, and (−)-(S)—N-[2-hydroxy-3-(1-piperidinyl)-propoxy]-pyridine-1-oxide-3-carboximidoyl chloride, (Z)—(R)—N-[2-hydroxy-3-(1-piperidinyl)-propoxy]-pyridine-1-oxide-3-carboximidoyl chloride, (E)-(R)—N-[2-hydroxy-3-(1 piperidinyl)-propoxy]-pyridine-1-oxide-3-carboximidoyl chloride, (Z)—(S)—N-[2-hydroxy-3-(1-piperidinyl)-propoxy]-pyridine-1-oxide-3-carboximidoyl chloride; and (E)-(S)—N-[2-hydroxy-3-(1-piperidinyl)-propoxy]-pyridine-1-oxide-3-carboximidoyl chloride.

76. The composition of 55-75, wherein the compound of Formula I to Formula VII is present as a racemate.

77. The composition of 55-76, wherein the compound of Formula 1 to Formula VII is present in a form selected from the group consisting of polymorphs, crystals, and combinations thereof.

78. The composition of 55-77, wherein the composition further comprises at least one excipient.

79. The composition of 78, wherein the at least one excipient is selected from the group consisting of antiadherents, binders, coatings, disintegrants, gel forming agents, fillers, flavors and colors, glidants, lubricants, preservatives, sorbents and sweeteners.

80. The composition of 55-79, wherein the at least one additional active pharmaceutical ingredient is selected from the group consisting of arimoclomol bimoclomol, miglustat, eliglustat, N-acetyl-L-leucine and 2-hydroxypropyl-β-cyclodextrin, and combinations thereof.

81. The composition of 80, wherein the at least one additional active pharmaceutical ingredient is selected from arimoclomol, miglustat, N-acetyl-L-leucine, and combinations thereof.

82. The composition of 81, wherein the at least one additional active pharmaceutical ingredient is a combination of arimoclomol, miglustat and N-acetyl-L-leucine.

83. The composition of 55-82, wherein the composition is formulated for oral administration.

84. The composition of 83, wherein the composition is a solid oral dosage formulation.

85. The composition of 84, wherein the solid oral dosage formulation is selected from the group consisting of a sublingual, a gummy, a chewable tablet, a rapidly dissolving tablet, a tablet, a capsule, a caplet, a troche, a lozenge, an oral powder, a solution, a thin strip, an oral thin film (OTF), an oral strip, and a rectal film.

86. The composition of 55-85, wherein the composition additionally comprises at least one release controlling agent selected from the group consisting of a polymer coating, a polymer matrix, or a combination thereof.

87. The composition of 86, wherein the at least one extended-release agent is a polymer coating selected from the group consisting of polyethylene oxides (PEO), hydroxypropyl cellulose (HPC), methylcellulose (MC), hydroxypropyl methylcellulose (HPMC), hydroxyethyl cellulose (HEC), ethylcellulose (EC), and sodium carboxymethyl cellulose (CMC)

88. The composition of 86, wherein the at least one extended-release agent is a polymer matrix selected from the group consisting of hydroxypropylmethylcellulose (HPMC), ethylcellulose (EC), methylcellulose, hypromellose acetate succinate, hypromellose phthalate, cellulose acetate, glycerin monostearate, glyceryl monooleate, glyceryl palmitate, glyceryl behenate, hydrogenated vegetable oil, guar gum, polyvinyl alcohol, alginates, xanthan gum, carnauba wax, yellow wax, white wax, zein, carrageenan, carbomers and agar.

89. The composition of 86-88, wherein the at least one extended-release agent is a combination of a polymer coating and a polymer matrix.

90. The composition of 55-83, wherein the composition is a liquid oral dosage formulation.

91. The composition of 90, wherein the liquid oral dosage formulation is selected from the group consisting of syrups, emulsions, solutions, and suspensions.

92. The composition of 55-91, wherein the therapeutically effective amount of the compound of Formula I to Formula VII ranges from about 1 to about 5000 mg per day.

93. The composition of 70, wherein the therapeutically effective amount of the compound of Formula I to Formula VII is administered as a unit dose.

94. A method of treating a neurodegenerative or metabolic disease or disorder in a patient in need thereof, the method comprising:
administering to the patient in need thereof a composition comprising a therapeutically effective amount of a compound having a structure of Formula I:

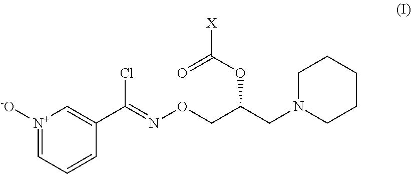

wherein X is selected from the group consisting of R, [O—R], [NR¹R²], and [NRH]
wherein O is oxygen, N is nitrogen, H is hydrogen,
and R, R¹ and R² are independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol,
or a pharmaceutically acceptable salt thereof.

95. The method of 94, wherein the compound of Formula I is a compound having a structure of Formula IA:

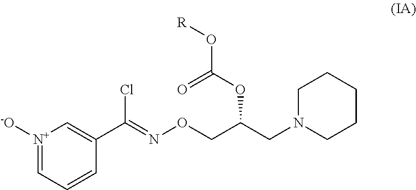

wherein R is selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol; or
a pharmaceutically acceptable salt thereof.

96. The method of 95, wherein the compound of Formula IA has a structure selected from the group consisting of:

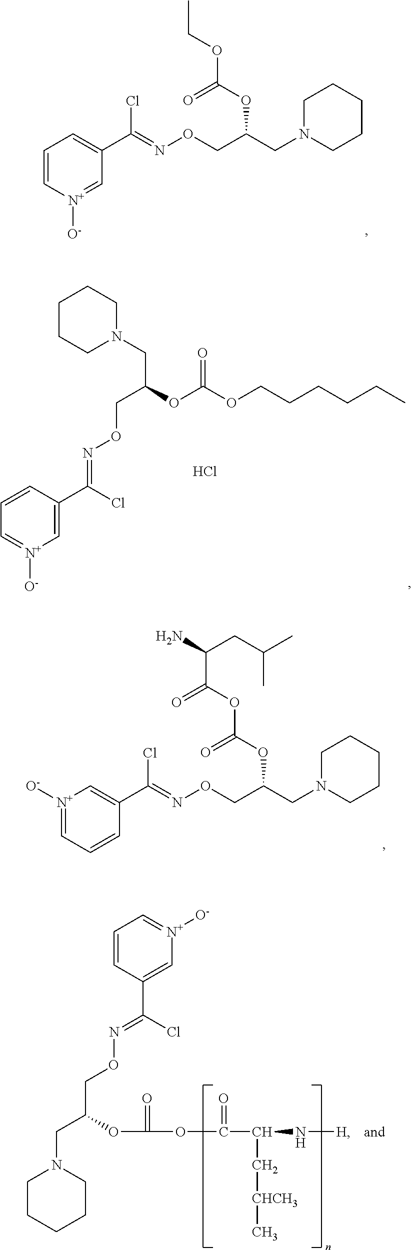

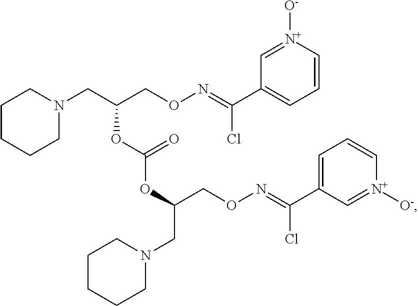

wherein n is 1-50
or a pharmaceutically acceptable salt thereof.

97. The method of 94, wherein the compound of Formula I is a compound having a structure of Formula IB:

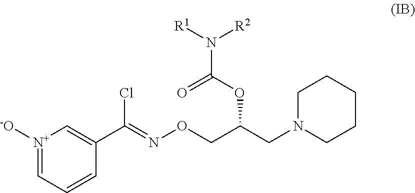

wherein R¹ and R2 are independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

98. The method of 97, wherein the compound of Formula IB has a structure selected from the group consisting of:

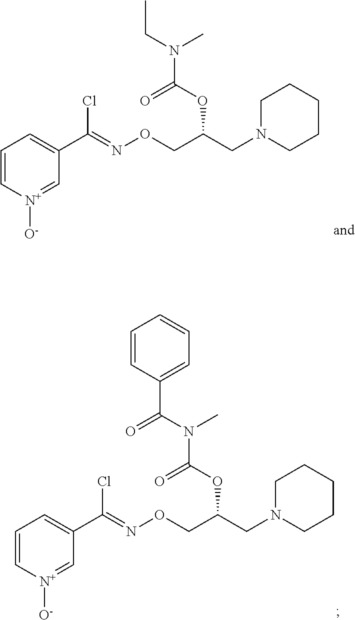 [SMILES: CCN(C)C(=O)O[C@@H](CO/N=C(\Cl)c1ccc[n+]([O-])c1)CN1CCCCC1 ; CN(C(=O)O[C@@H](CO/N=C(\Cl)c1ccc[n+]([O-])c1)CN1CCCCC1)C(=O)c1ccccc1]

or a pharmaceutically acceptable salt thereof.

99. The method of 94, wherein the compound of Formula I is a compound of Formula IC:

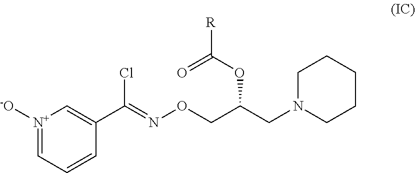

wherein R is selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

100. The method of 99, wherein the compound of Formula IC has a structure selected from the group consisting of:

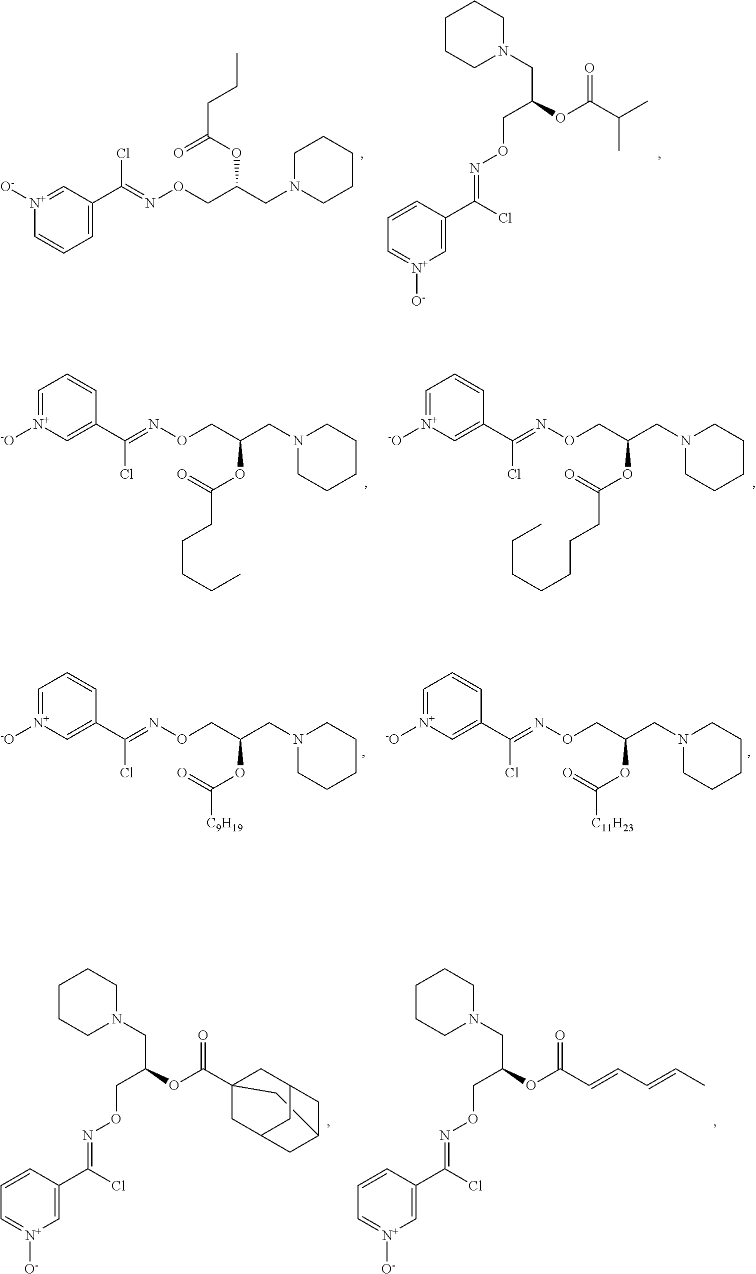

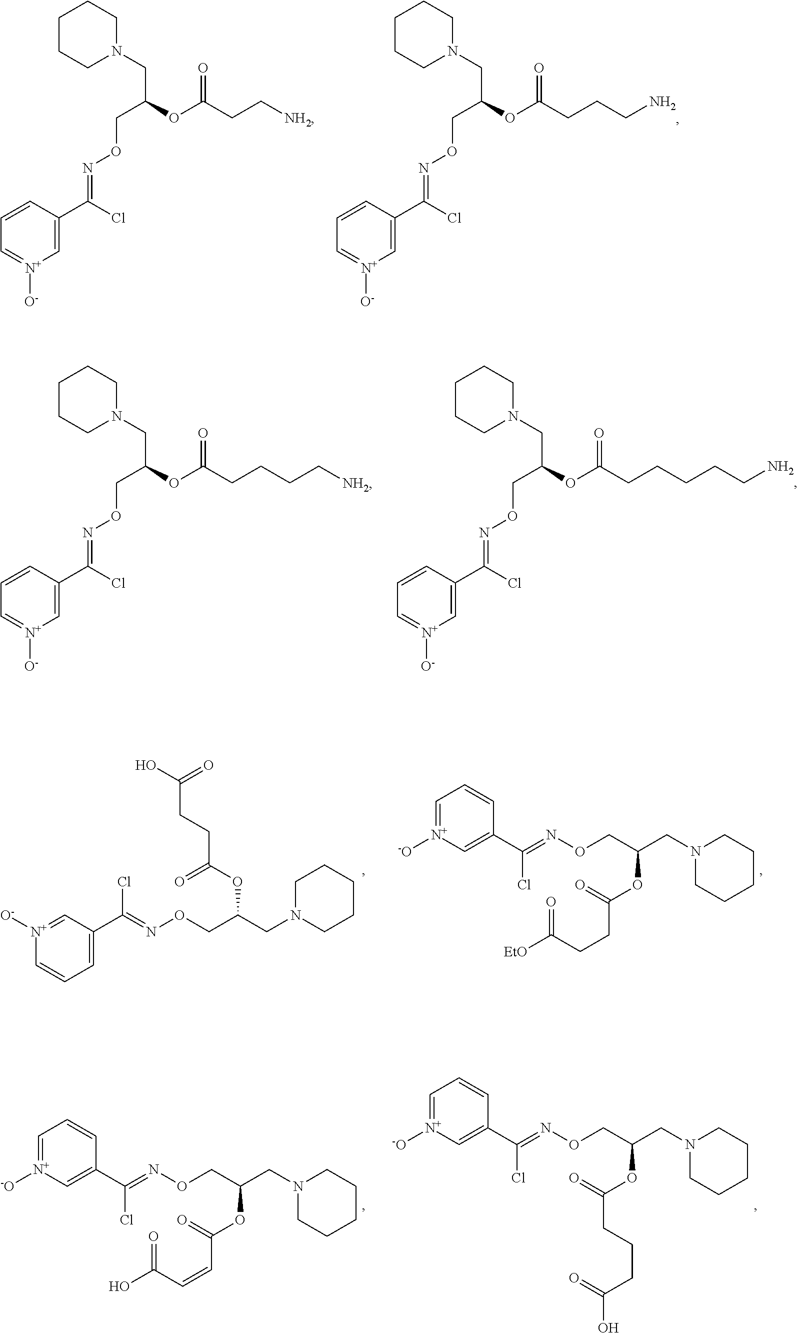

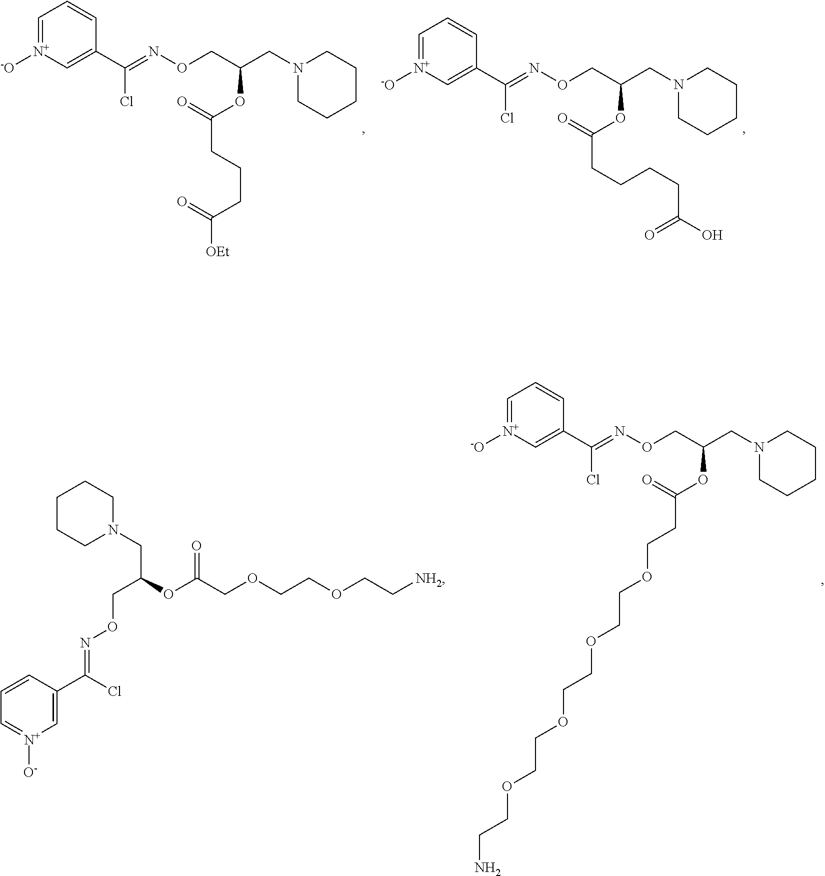

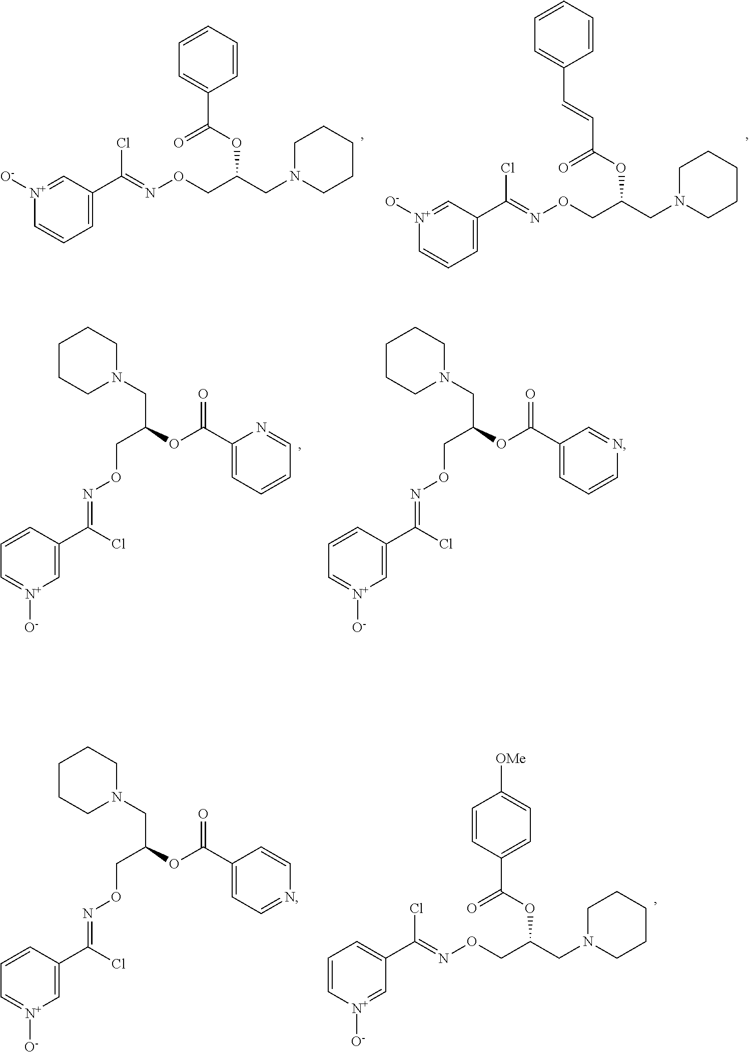

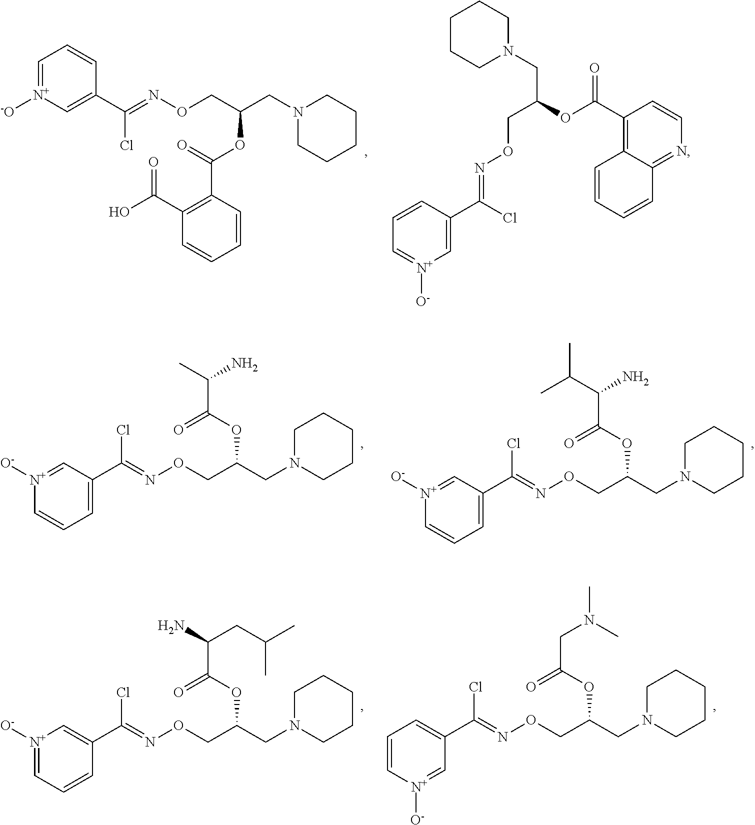

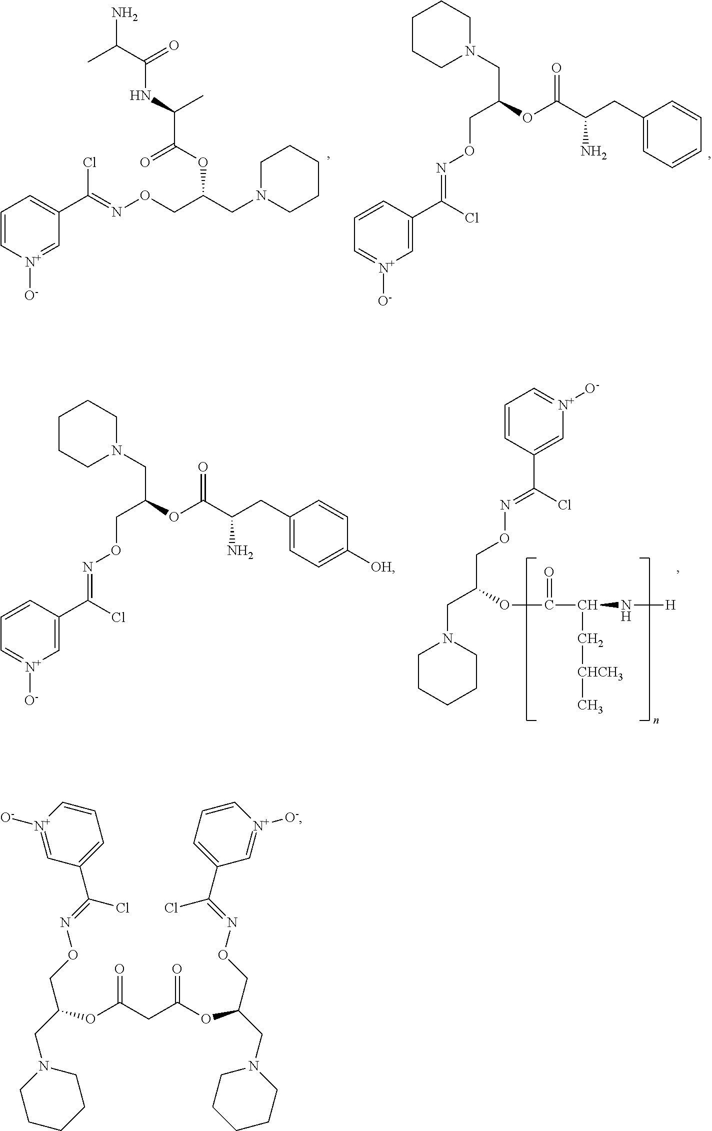

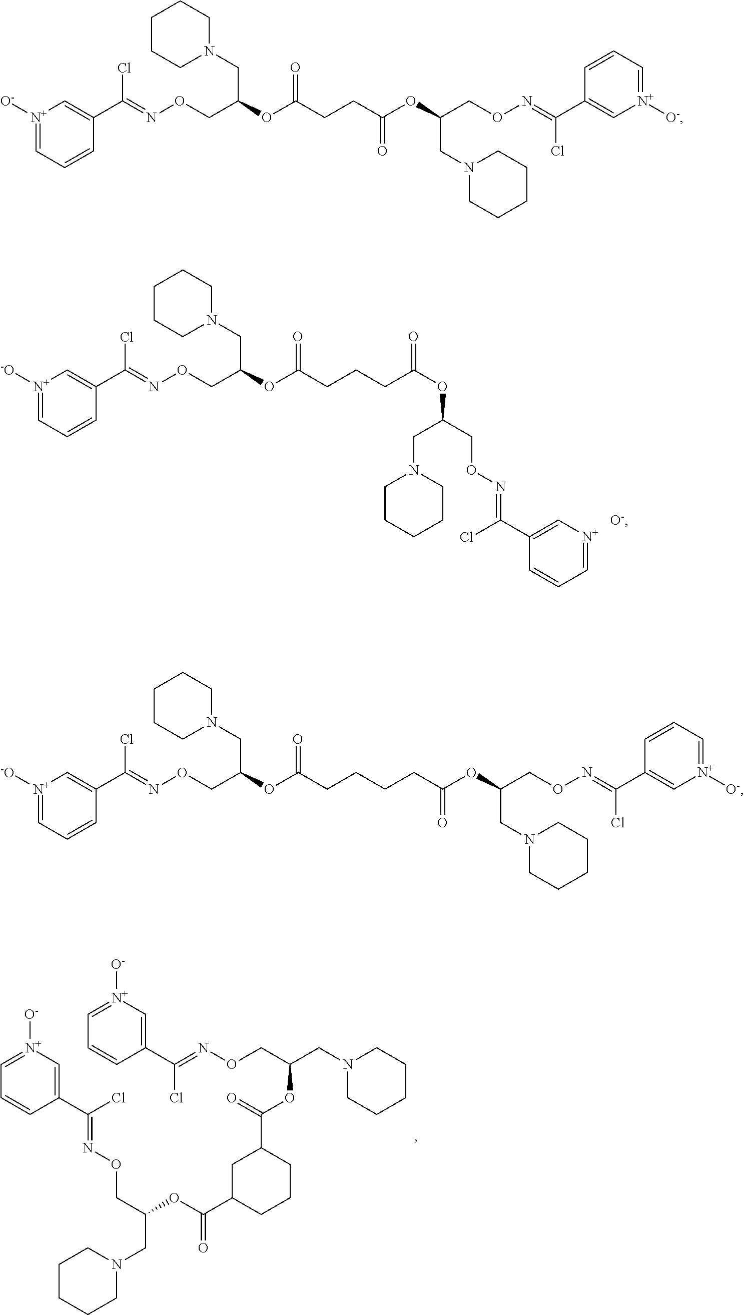

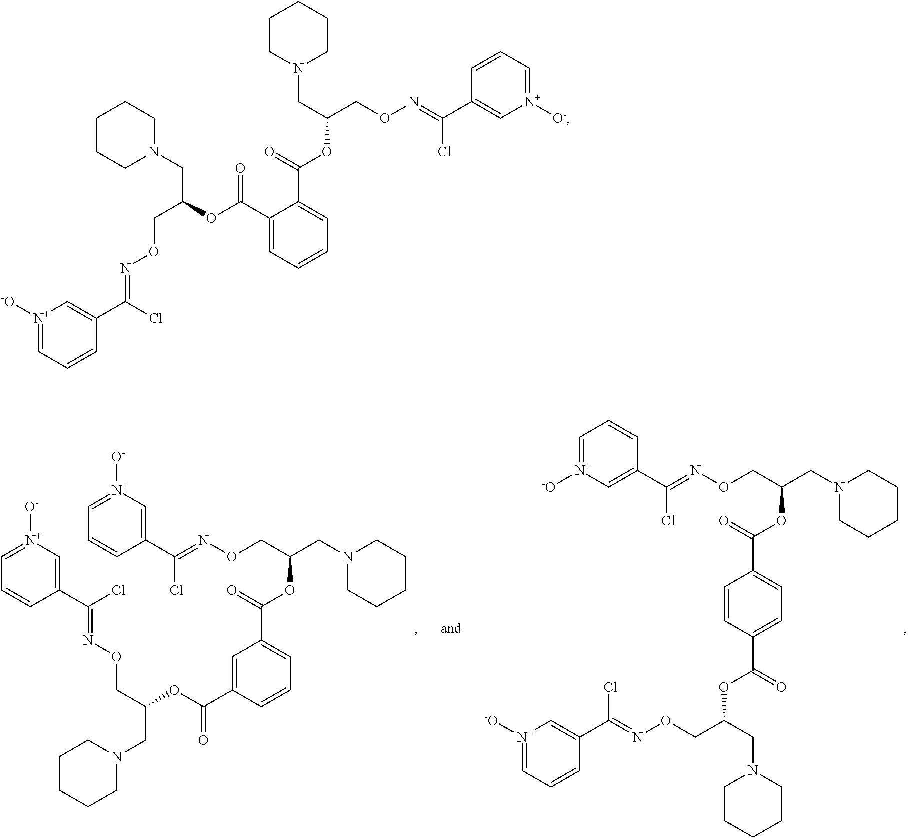

wherein n is 1-50;
or a pharmaceutically acceptable salt thereof.

101. The method of 100, wherein the compound of Formula IC is selected from the group consisting of:

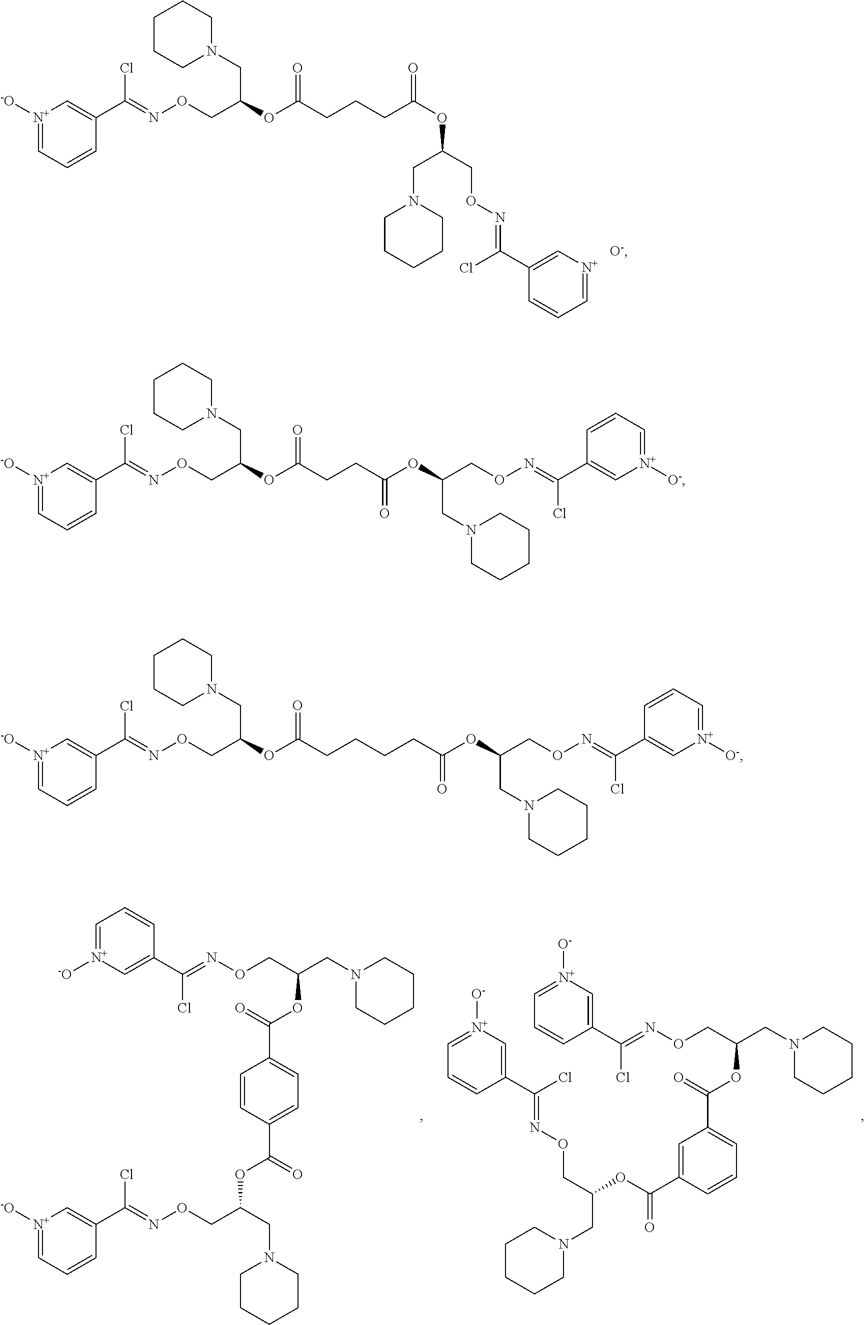

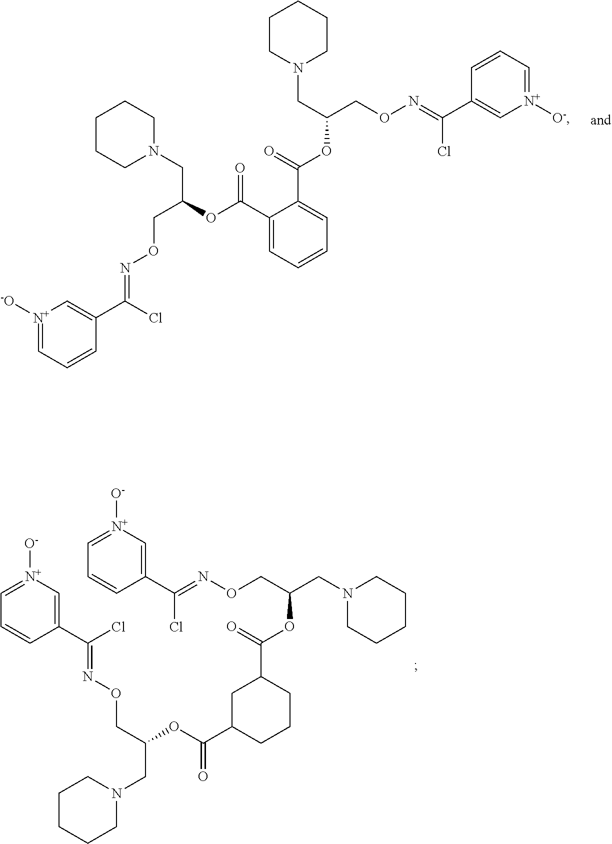

or a pharmaceutically acceptable salt thereof.

102. The method of 94, wherein the compound of Formula I is a compound of Formula ID:

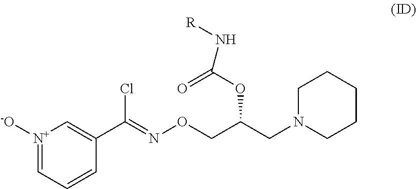

wherein R is selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

103. The method of 102, wherein the compound of Formula ID is a compound having a structure selected from the group consisting of:

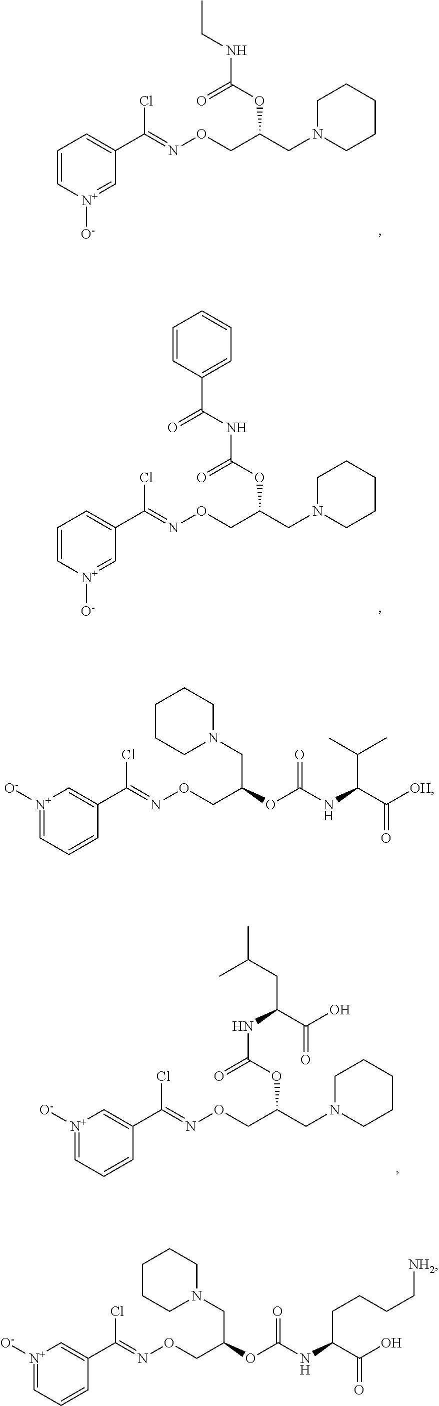

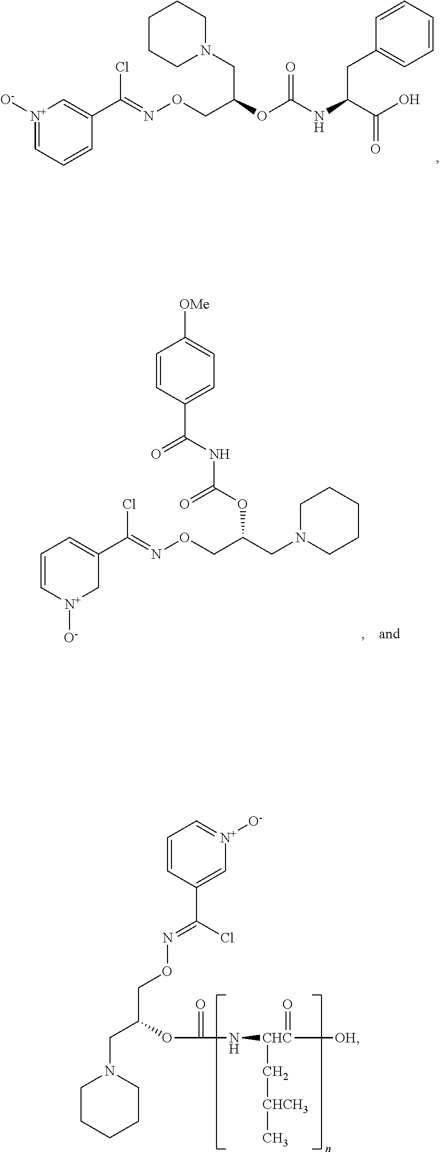

wherein n is 1-50;
or a pharmaceutically acceptable salt thereof.

105. A method of treating a neurodegenerative or metabolic disease or disorder in a patient in need thereof, the method comprising:
administering to the patient in need thereof a composition comprising a therapeutically effective amount of a compound having a structure of Formula II:

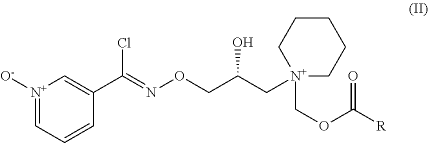

wherein R is independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

106. The compound of 105, wherein the compound of Formula II has a structure selected from the group consisting of:

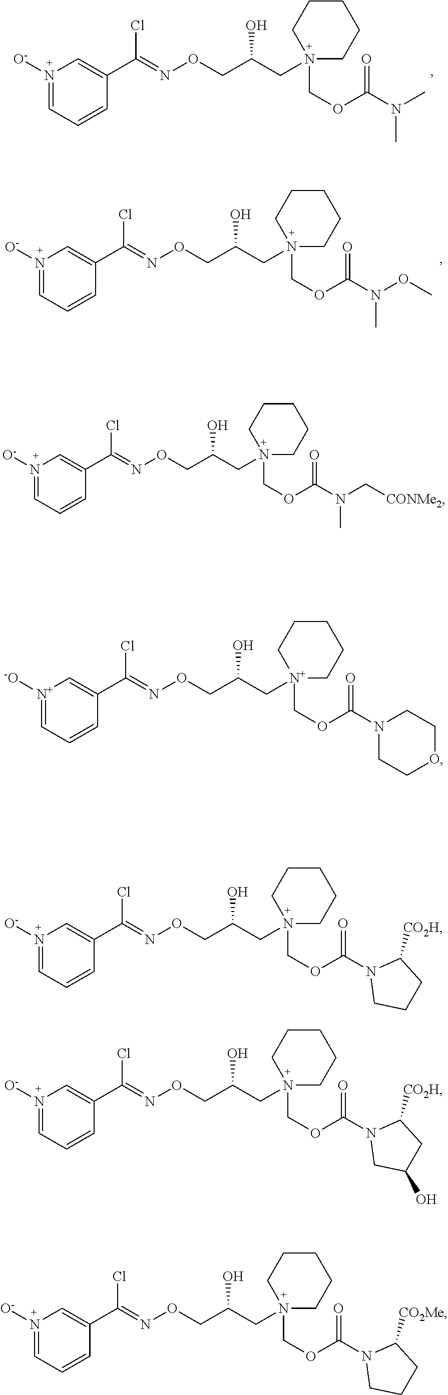

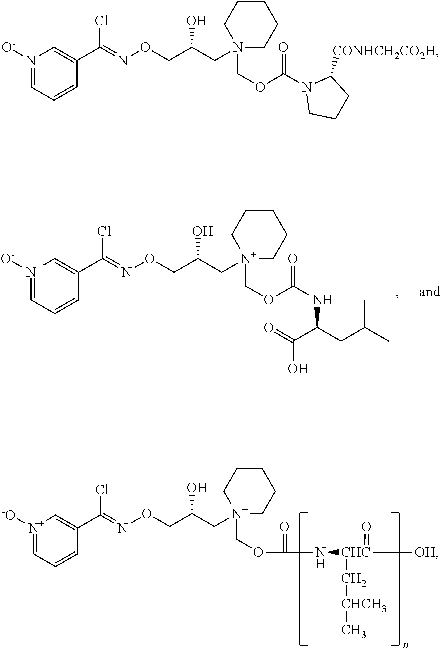

wherein n is 1-50;
or a pharmaceutically acceptable salt thereof

107. A method of treating a neurodegenerative or metabolic disease or disorder in a patient in need thereof, the method comprising:
administering to the patient in need thereof a composition comprising a therapeutically effective amount of a compound having a structure of Formula III:

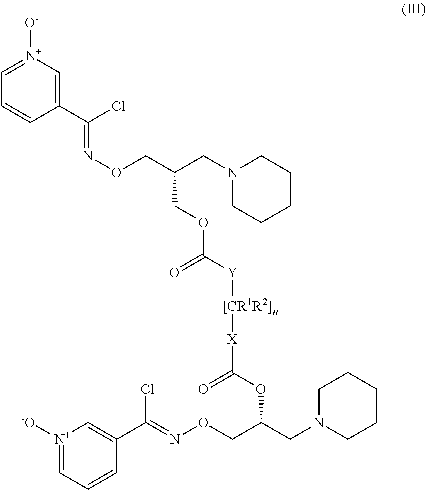

wherein X is selected from the group consisting of O, N, or absent,
Y is selected from the group consisting of O, N, or absent,
n is 1-10,
R¹ and R² are selected independently from each other and for each CR¹R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

108. A method of treating a neurodegenerative or metabolic disease or disorder in a patient in need thereof, the method comprising:
administering to the patient in need thereof a composition comprising a therapeutically effective amount of a compound having a structure of Formula IV:

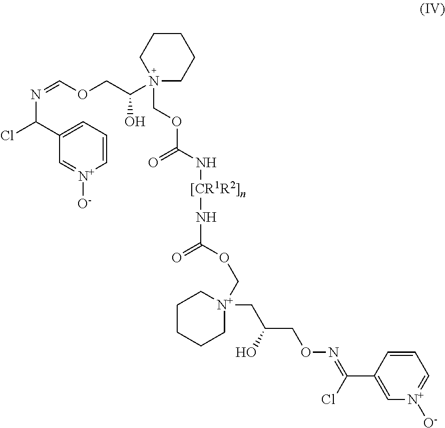

n is 1-10,
R¹ and R² are selected independently from each other and for each CR₁R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

109. A method of treating a neurodegenerative or metabolic disease or disorder in a patient in need thereof, the method comprising:
administering to the patient in need thereof a composition comprising a therapeutically effective amount of a compound having a structure of Formula V

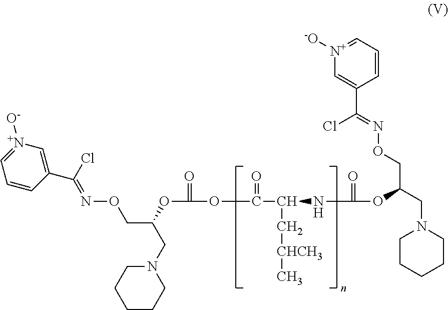

wherein n is 1-50,
or a pharmaceutically acceptable salt thereof.

110. A method of treating a neurodegenerative or metabolic disease or disorder in a patient in need thereof, the method comprising:
administering to the patient in need thereof a composition comprising a therapeutically effective amount of a compound having a structure of Formula VI:

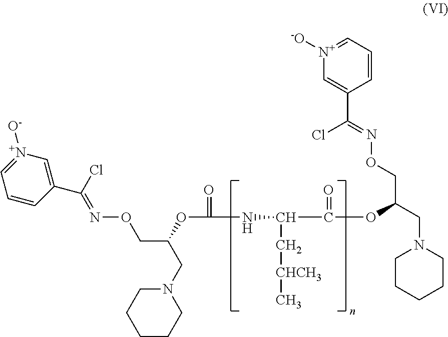

wherein n is 1-50;
or a pharmaceutically acceptable salt thereof.

111. A method of treating a neurodegenerative or metabolic disease or disorder in a patient in need thereof, the method comprising:
administering to the patient in need thereof a composition comprising a therapeutically effective amount of a compound having a structure of Formula VII:

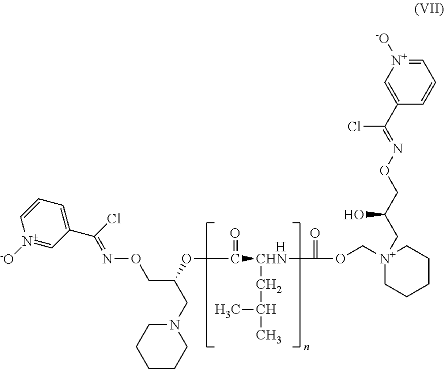

wherein n is 1-50,
or a pharmaceutically acceptable salt thereof.

112. A method of treating a neurodegenerative or metabolic disease or disorder in a patient in need thereof, the method comprising:
administering to the patient in need thereof a composition comprising a therapeutically effective amount of a compound having a structure selected from the group consisting of:

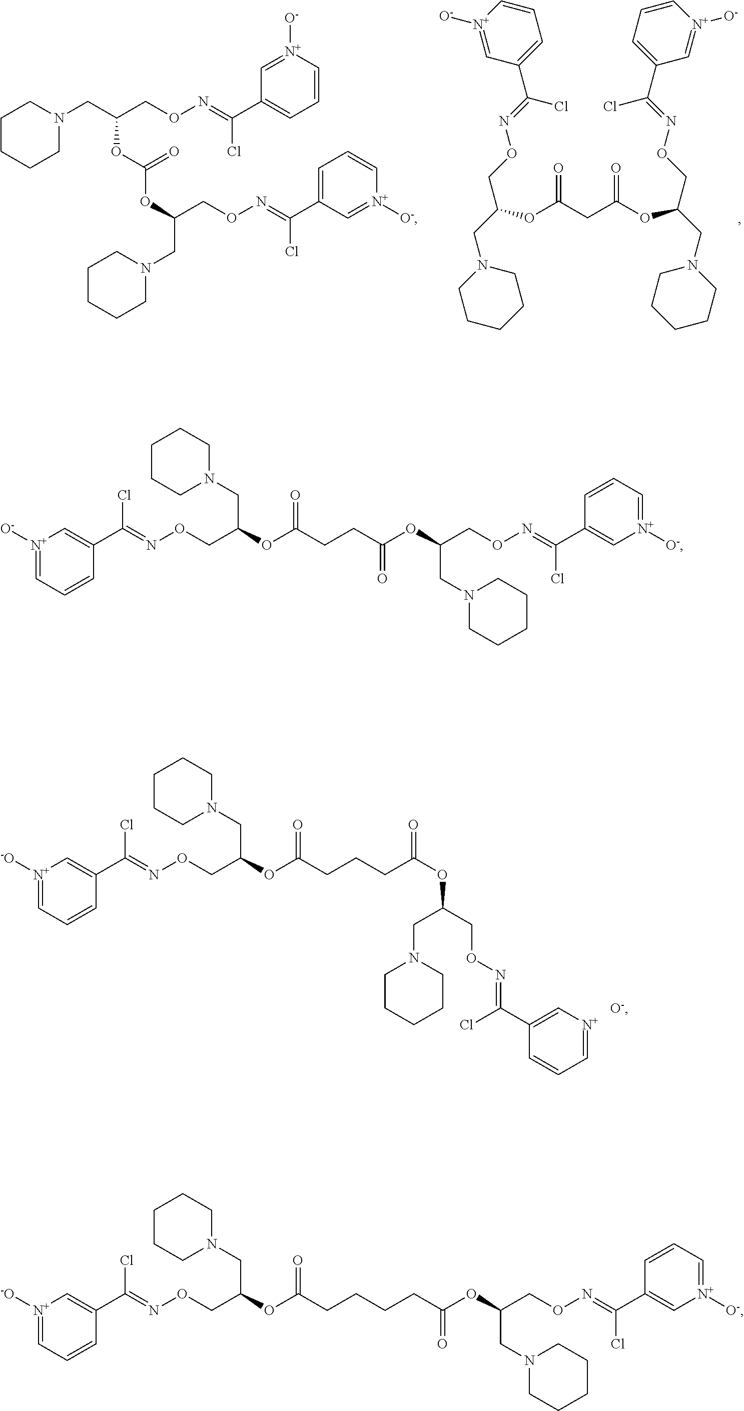

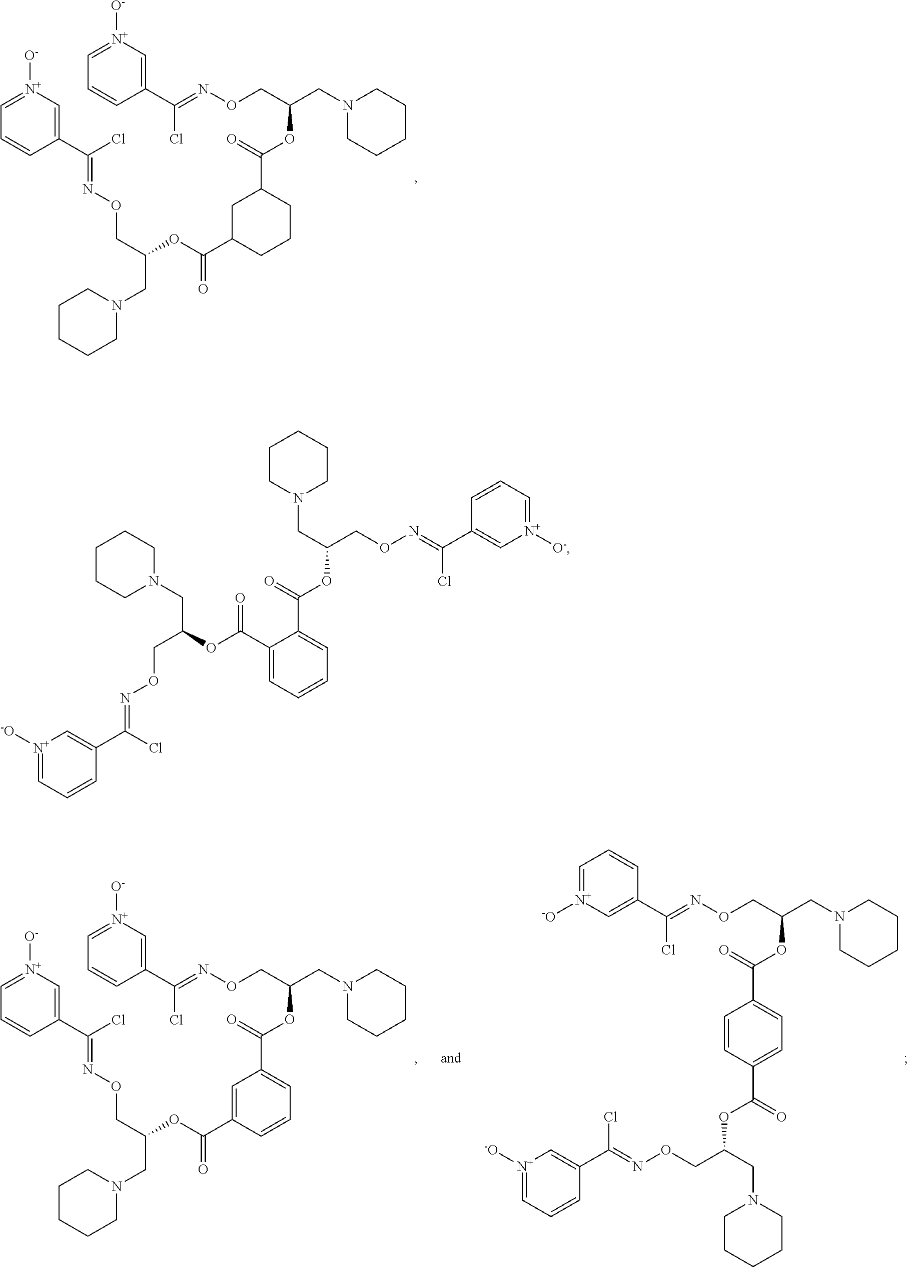

or a pharmaceutically acceptable salt thereof.

113. A method of treating a neurodegenerative or metabolic disease or disorder in a patient in need thereof, the method comprising:
administering to the patient in need thereof a composition comprising a therapeutically effective amount of a compound having a structure selected from the group consisting of:

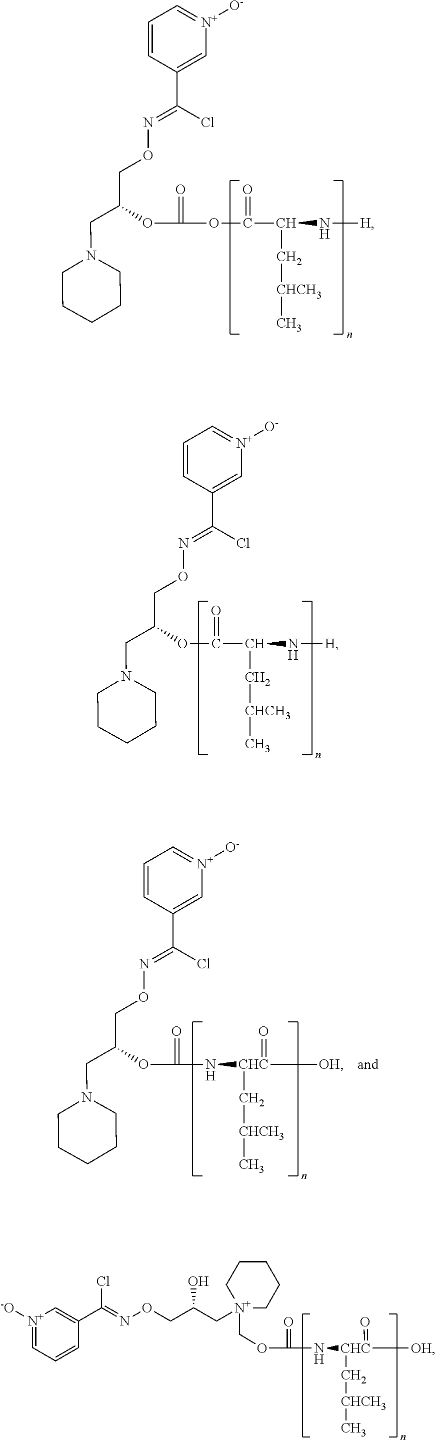

wherein n is 1-50;
or a pharmaceutically acceptable salt thereof.

114. The method of 94-113, wherein the pharmaceutically acceptable salt is selected from: sulfate, citrate, acetate, oxalate, chloride, hydrochloride, bromide, hydrobromide, iodide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

115. The method of 94-114, wherein the neurodegenerative or metabolic disease or disorder is a lysosomal storage disorder.

116. The method of 115, wherein the lysosomal storage disorder is selected from the group consisting of sphingolipidoses, mucopolysaccharidoses (MPS), lysosomal glycogen storage disorders, oligosaccharidoses, mucolipidoses, lipid storage disorders, glycoproteinoses, neuronal ceroid lipofuscinoses (NCL), peroxisomal biogenesis disorders (Zellweger spectrum disorders), and other lysosomal storage disorders.

117. The method of 116, wherein the sphingolipidosis is selected from the group consisting of Gaucher disease type 1, Gaucher disease type 2, Gaucher disease type 3, Fabry disease, Niemann-pick disease type A, Niemann-pick disease type B, Niemann-pick disease type C, Tay-Sachs disease, Sandhoff disease, GM1 gangliosidosis, metachromatic leukodystrophy (MLD), Krabbe disease, and Farber lipogranulomatosis.

118. The method of 116, wherein the mucopolysaccharidosis is selected from the group consisting of MIPS I, Hurler syndrome, Hurler-Scheie syndrome, Scheie syndrome, MPS II, Hunter syndrome), MPS III, Sanfilippo syndrome type A, Sanfilippo syndrome type B, Sanfilippo syndrome type C, Sanfilippo syndrome type D, MPS IV, Morquio syndrome type A, Morquio syndrome type B), MPS VI, Maroteaux-Lamy syndrome, MPS VII, Sly syndrome, MPS IX, and Natowicz syndrome.

119. The method of 116, wherein the lysosomal glycogen storage disorder is selected from the group consisting of Pompe disease and glycogen storage disease type II.

120. The method of 116, wherein the oligosaccharidosis is selected from the group consisting of Schindler disease, Sialidosis Type I, Sialidosis type II, aspartylglucosaminuria, alpha-mannosidosis, beta-mannosidosis, and fucosidosis.

121. The method of 116, wherein the mucolipidosis is selected from the group consisting of mucolipidosis II, I-cell disease, mucolipidosis III, pseudo-Hurler polydystrophy, and mucolipidosis IV.

122. The method of 116, wherein the lipid storage disorder is selected from the group consisting of Wolman disease, cholesteryl ester storage disease, and Farber disease.

123. The method of 116, wherein the glycoproteinosis is selected from the group consisting of alpha-mannosidosis, beta-mannosidosis, fucosidosis, aspartylglucosaminuria.

124. The method of 116, wherein the neuronal ceroid lipofuscinosis is selected from the group consisting of infantile NCL, CLN1, CLN5, late infantile NCL, CLN2, juvenile NCL, CLN3, adult NCL, Kufs disease, CLN4, congenital NCL, and CLN10.

125. The method of 116, wherein the peroxisomal biogenesis disorder is selected from the group consisting of Zellweger syndrome, neonatal adrenoleukodystrophy, and infantile Refsum disease.

126. The method of 116, wherein the other lysosomal storage disorder is selected from the group consisting of Danon disease, cystinosis, sialic acid storage disease, Salla disease, lysosomal acid lipase deficiency (LAL-D), multiple sulfatase deficiency, pycnodysostosis, galactosialidosis, hyaluronidase deficiency (MPS IX), Hermansky-Pudlak syndrome, and pseudoxanthoma elasticum (PXE).

127. The method of 117, wherein the Niemann Pick disease Type C is Niemann Pick disease Type C 1 or Niemann Pick disease Type C 2.

128. The method of 94-125, wherein the therapeutically effective amount of a compound of Formula I to Formula VII is between 1 mg and 5000 mg per day.

129. The method of 128, wherein the therapeutically effective amount of a compound of Formula I to Formula VII is between 500 mg and 2000 mg.

130. The method of 94-129, wherein the therapeutically effective amount is administered in 1 to 3 unit doses per day.

131. The method of 130, wherein the therapeutically effective amount is administered in 1 unit dose per day.

132. The method of 94-131, wherein the unit dose is an oral formulation.

133. The method of 132, wherein the oral formulation is a solid oral formulation.

134. The method of 133, wherein the solid oral formulation is selected from the group consisting of a tablet, a capsule, a powder, a film, a lozenge, and a micro-capsule.

135. The method of 132, wherein the composition is a liquid oral dosage formulation.

136. The composition of 135, wherein the liquid oral dosage formulation is selected from the group consisting of syrups, emulsions, solutions, and suspensions.

137. The method of 94-136, wherein the method comprises administering to the patient in need thereof a composition comprising a therapeutically effective amount of at least one additional active pharmaceutical agent selected from the group consisting of: arimoclomol, bimoclomol. miglustat, eligustat, N-acetyl-L-leucine 2-hydroxypropyl-β-cyclodextrin, and combinations thereof.

138. The method of 94-137, wherein the patient in need thereof is aged 2 years or less.

139. A method of treating Niemann-Pick Disease, type C, in a patient in need thereof, the method comprising:
administering to the patient in need thereof a composition comprising a therapeutically effective amount of a compound of Formula I:

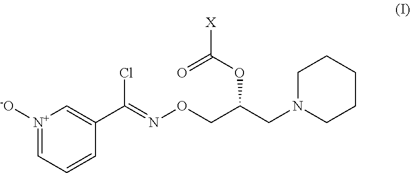

wherein X is selected from the group consisting of R, [O—R], [NR¹R²], and [NRH],
wherein O is oxygen, N is nitrogen, H is hydrogen, and
R, R¹ and R² are independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof; and
administering to the patient in need thereof a composition comprising a therapeutically effective amount of miglustat, or a pharmaceutically acceptable salt thereof.

140. A method of treating Niemann-Pick Disease, type C, in a patient in need thereof, the method comprising:
administering to the patient in need thereof a composition comprising a therapeutically effective amount of a compound of Formula II:

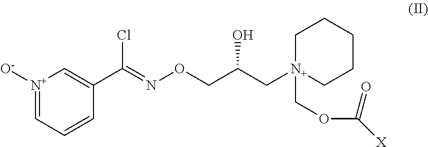

wherein X is selected from the group consisting of R, [O—R], [NR¹R²], and [NRH],
wherein O is oxygen, N is nitrogen, H is hydrogen, and
R, R¹ and R² are independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof; and
administering to the patient in need thereof a composition comprising a therapeutically effective amount of miglustat, or a pharmaceutically acceptable salt thereof.

141. A method of treating Niemann-Pick Disease, type C, in a patient in need thereof, the method comprising:
administering to the patient in need thereof a composition comprising a therapeutically effective amount of a compound of Formula III:

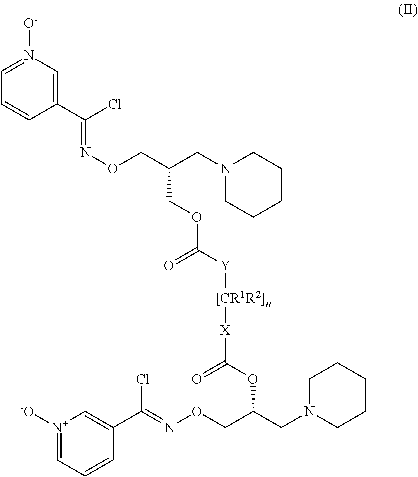

wherein X is selected from the group consisting of R, [O—R], [NR¹R²], and [NRH],
wherein O is oxygen, N is nitrogen, H is hydrogen, and
wherein X is selected from the group consisting of O, N, or absent,
Y is selected from the group consisting of O, N, or absent,
n is 1-10,
R¹ and R² are selected independently from each other and for each CR¹R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof; and
administering to the patient in need thereof a composition comprising a therapeutically effective amount of miglustat, or a pharmaceutically acceptable salt thereof.

142. A method of treating Niemann-Pick Disease, type C, in a patient in need thereof, the method comprising:
administering to the patient in need thereof a composition comprising a therapeutically effective amount of a compound of Formula IV:

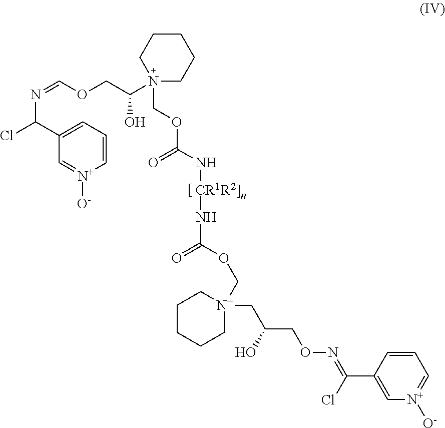

n is 1-10,
R¹ and R² are selected independently from each other and for each CR₁R² unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof; and
administering to the patient in need thereof a composition comprising a therapeutically effective amount of miglustat, or a pharmaceutically acceptable salt thereof.

143. A method of treating Niemann-Pick Disease, type C, in a patient in need thereof, the method comprising:
administering to the patient in need thereof a composition comprising a therapeutically effective amount of a compound of Formula V:

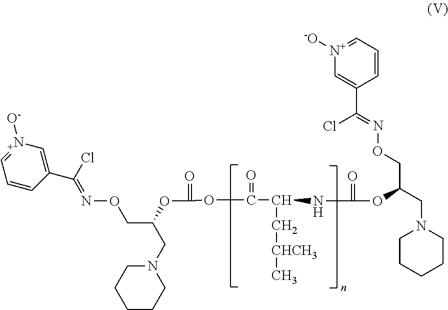

wherein n is 1-50,
or a pharmaceutically acceptable salt thereof; and
administering to the patient in need thereof a composition comprising a therapeutically effective amount of miglustat, or a pharmaceutically acceptable salt thereof.

144. A method of treating Niemann-Pick Disease, type C, in a patient in need thereof, the method comprising:
administering to the patient in need thereof a composition comprising a therapeutically effective amount of a compound of Formula VI:

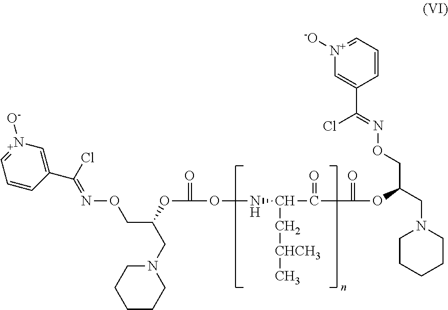

wherein n is 1-50,
or a pharmaceutically acceptable salt thereof; and
administering to the patient in need thereof a composition comprising a therapeutically effective amount of miglustat, or a pharmaceutically acceptable salt thereof.

145. A method of treating Niemann-Pick Disease, type C, in a patient in need thereof, the method comprising:
administering to the patient in need thereof a composition comprising a therapeutically effective amount of a compound of Formula VII:

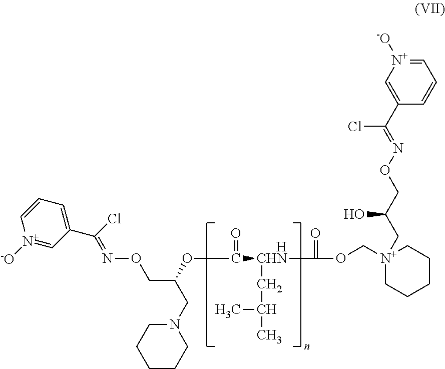

wherein n is 1-50,
or a pharmaceutically acceptable salt thereof; and
administering to the patient in need thereof a composition comprising a therapeutically effective amount of miglustat, or a pharmaceutically acceptable salt thereof.

146. The method of 139-145, wherein the patient in need thereof is 2 years of age or less.

The presently described technology is now described in such full, clear, concise and exact terms as to enable any person skilled in the art to which it pertains, to practice the same. It is to be understood that the foregoing describes preferred embodiments of the technology and that modifications may be made therein without departing from the spirit or scope of the invention as set forth in the appended claims.

1-148. (canceled)

149. A compound having a structure of Formula I: 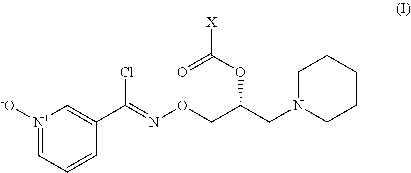
wherein X is selected from the group consisting of R, [O—R], [NR¹R²], and [NRH]
wherein O is oxygen, N is nitrogen, H is hydrogen, and
R, R¹ and R² are independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol,
or a pharmaceutically acceptable salt thereof.

150. The compound of claim 149, wherein the compound is a compound having a structure of Formula IA: 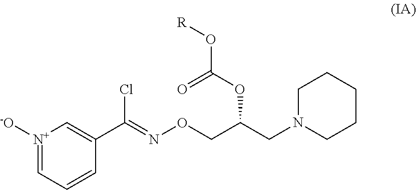
wherein R is selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol.

151. The compound of claim 150, wherein the compound of Formula IA has a structure selected from the group consisting of: 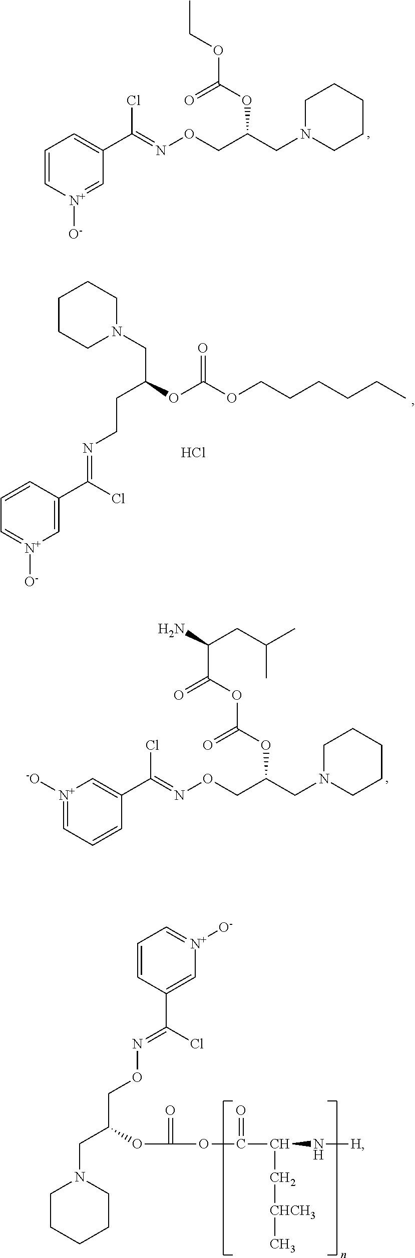 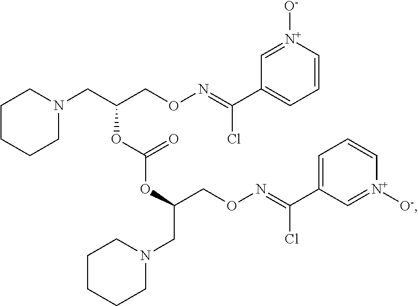
wherein n is 1-50;
or a pharmaceutically acceptable salt thereof.

152. The compound of claim 149, wherein the compound is a compound having a structure of Formula IB: 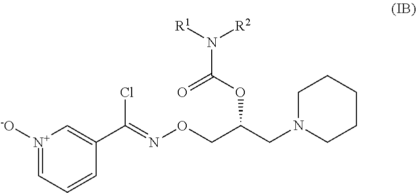
wherein R¹ and R² are independently selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

153. The compound of claim 152, wherein the compound of Formula IB has a structure selected from the group consisting of: 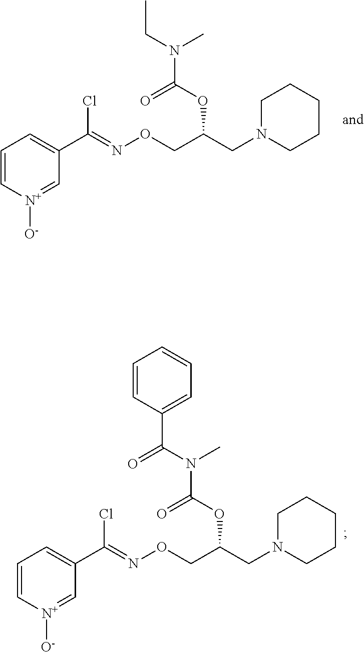 [SMILES: CCN(C)C(=O)O[C@@H](CO/N=C(\Cl)c1ccc[n+]([O-])c1)CN1CCCCC1 ; CN(C(=O)O[C@@H](CO/N=C(\Cl)c1ccc[n+]([O-])c1)CN1CCCCC1)C(=O)c1ccccc1]
or a pharmaceutically acceptable salt thereof.

154. The compound of claim 149, wherein the compound of Formula I is a compound of Formula IC: 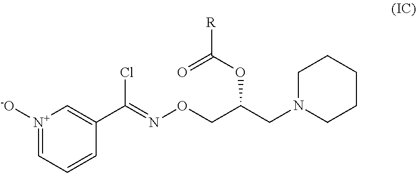
wherein R is selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

155. The compound of claim 154, wherein the compound of Formula IC has a structure selected from the group consisting of: 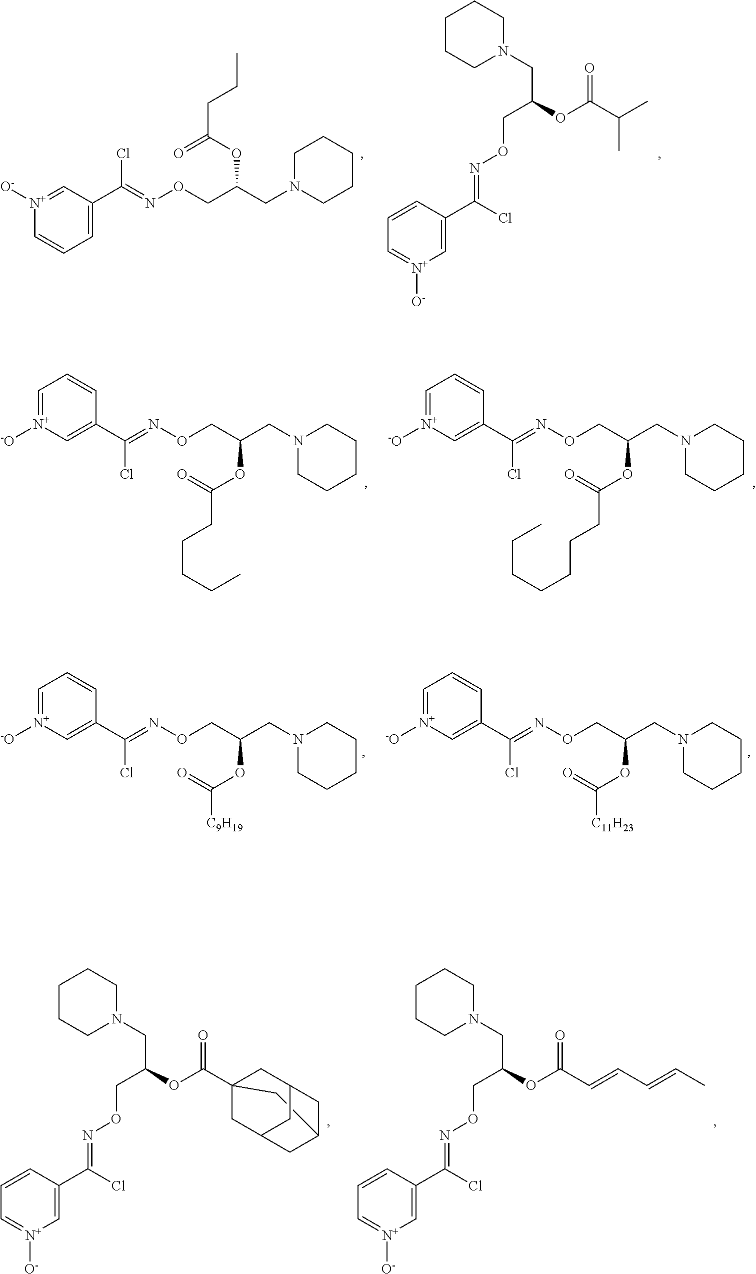 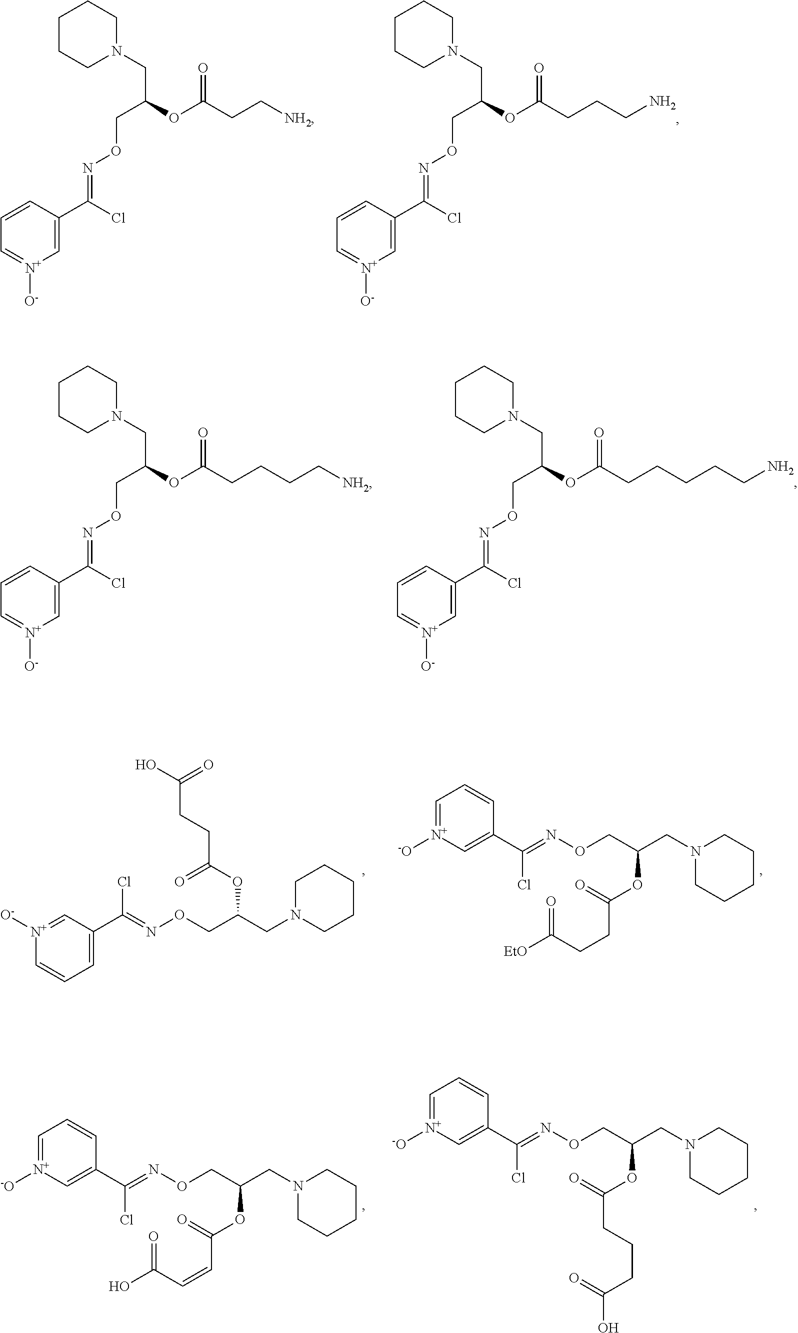 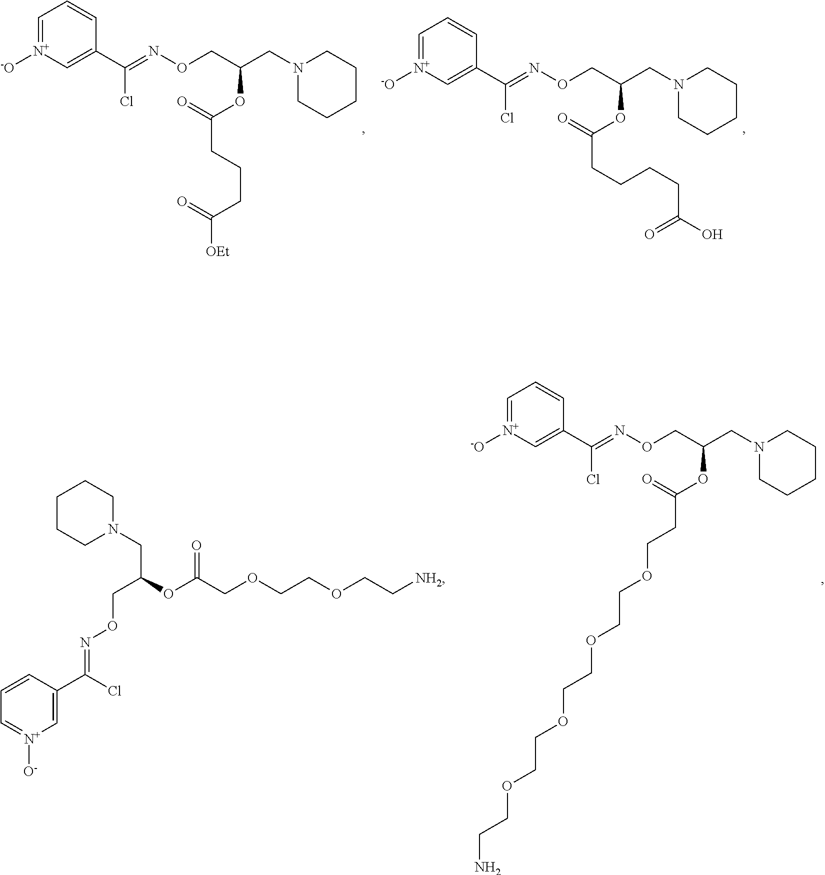 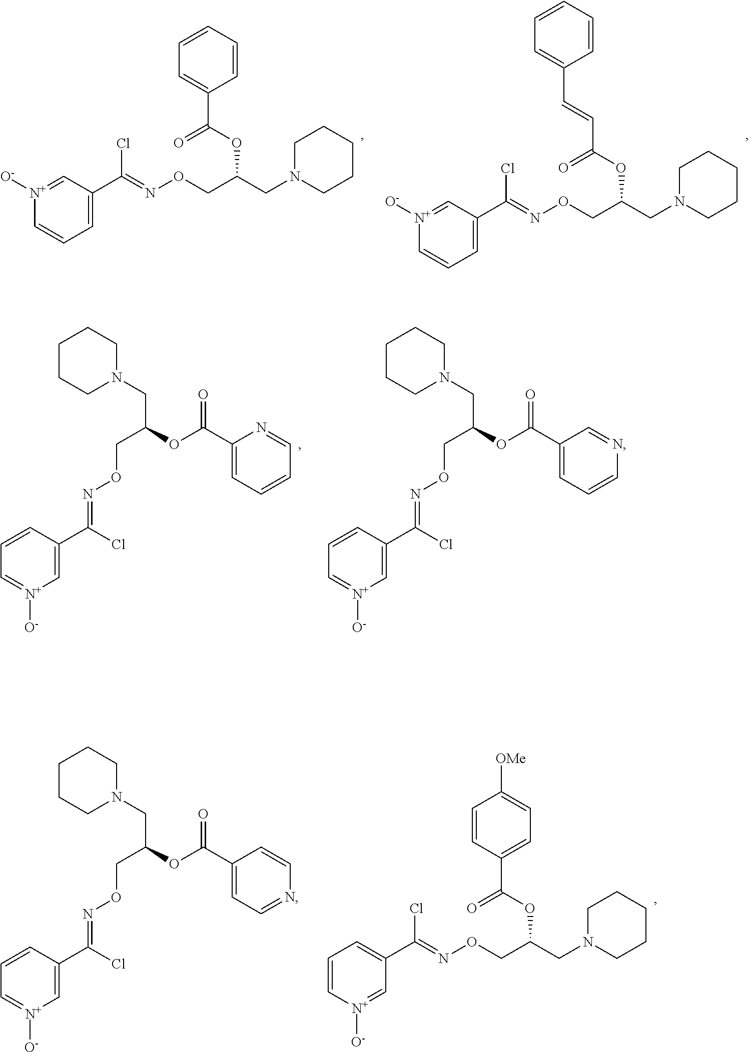 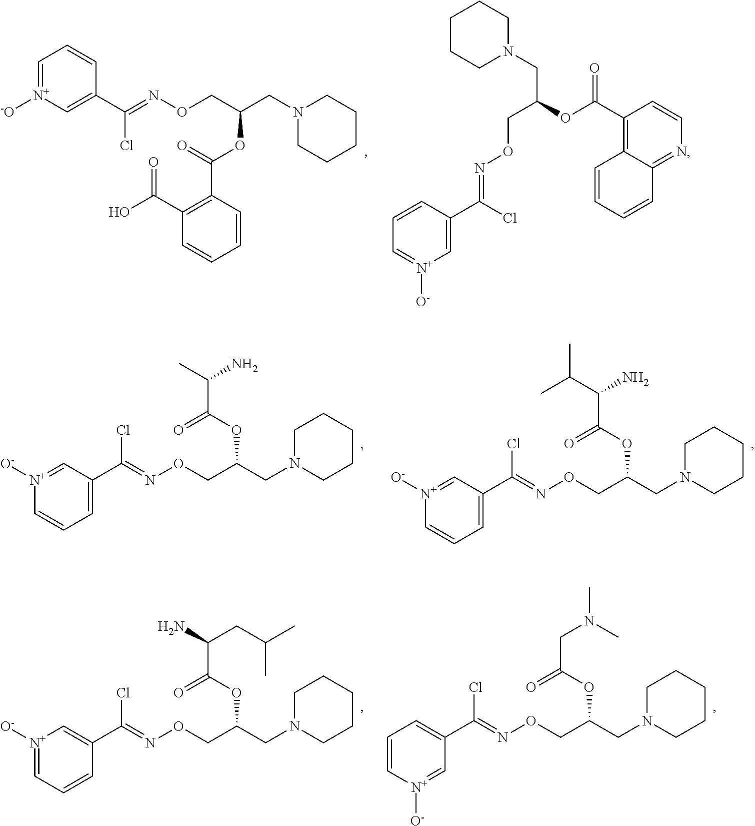 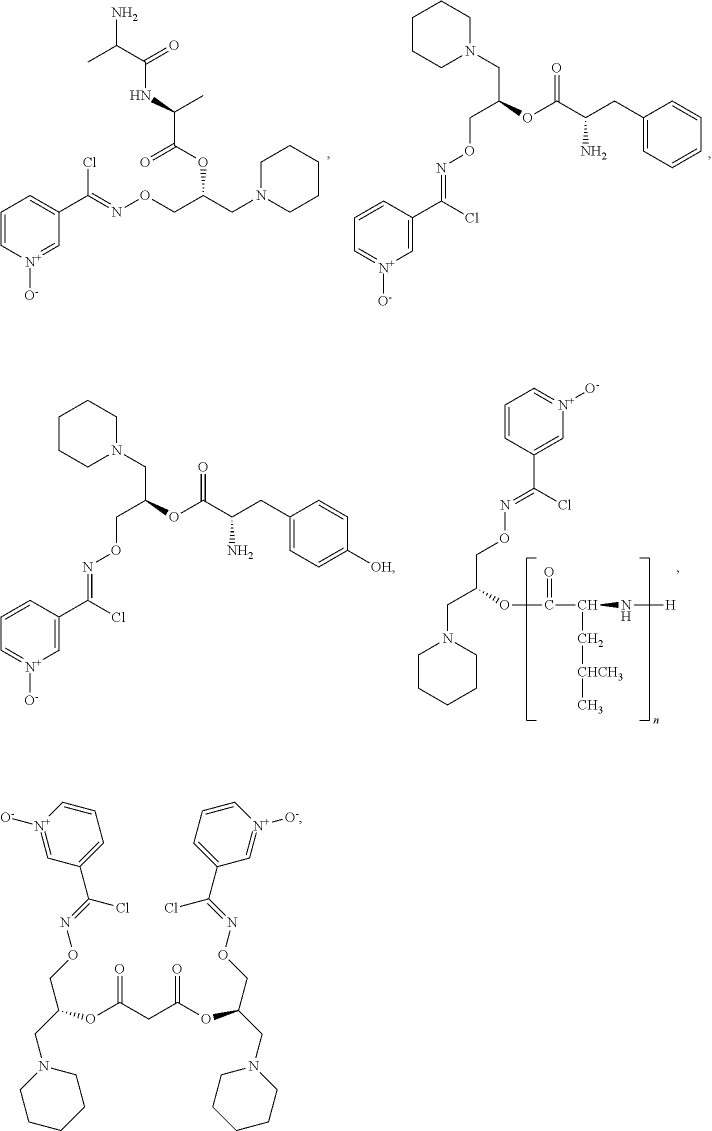 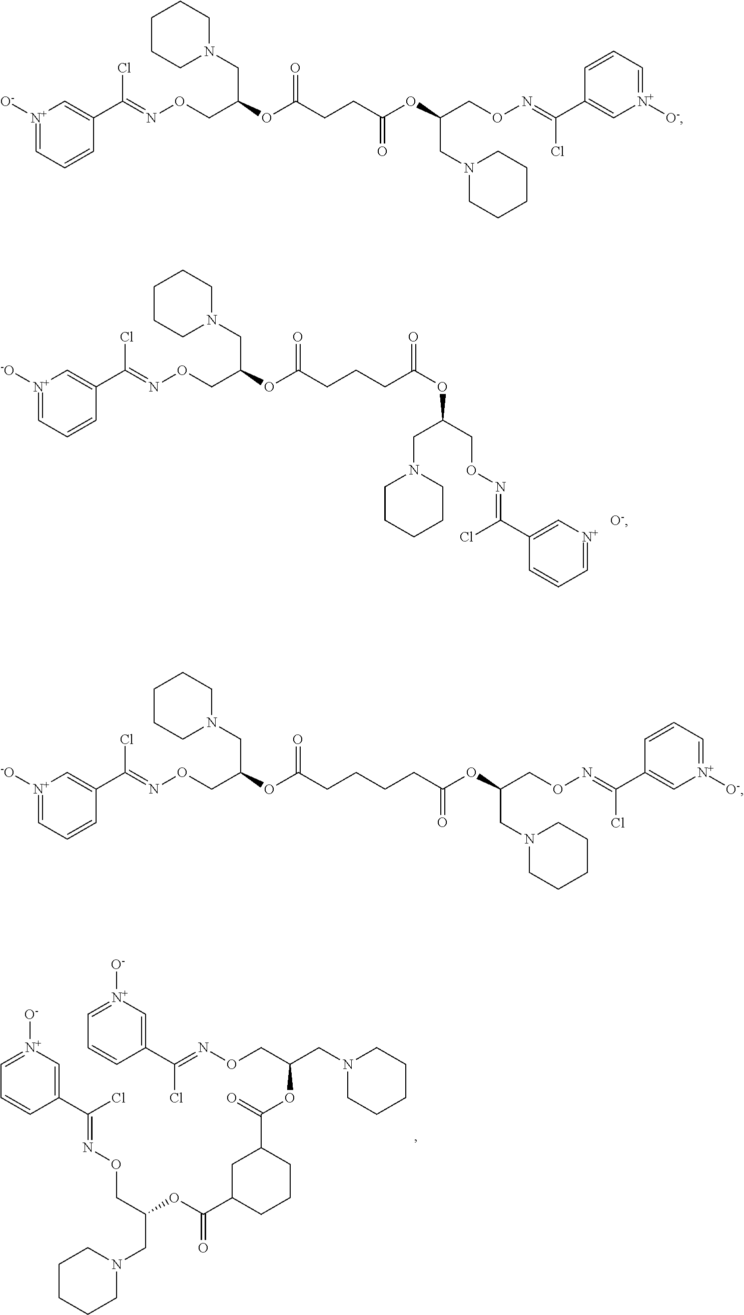 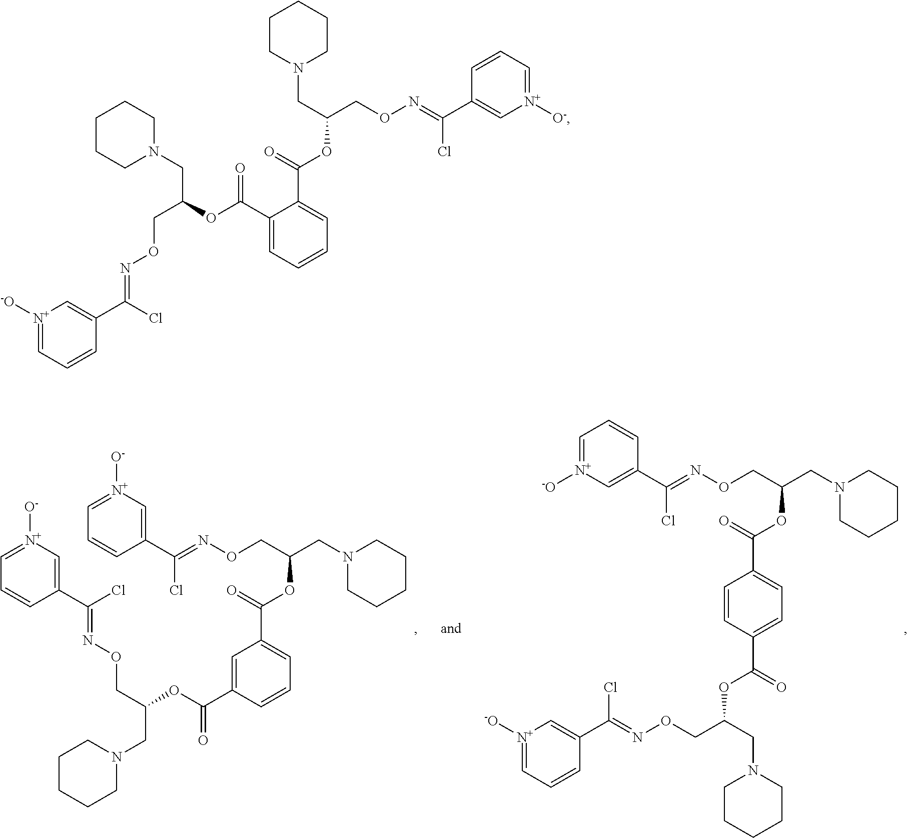
wherein n is 1-50;
or a pharmaceutically acceptable salt thereof.

156. The compound of claim 155, wherein the compound of Formula IC has a structure selected from the group consisting of: 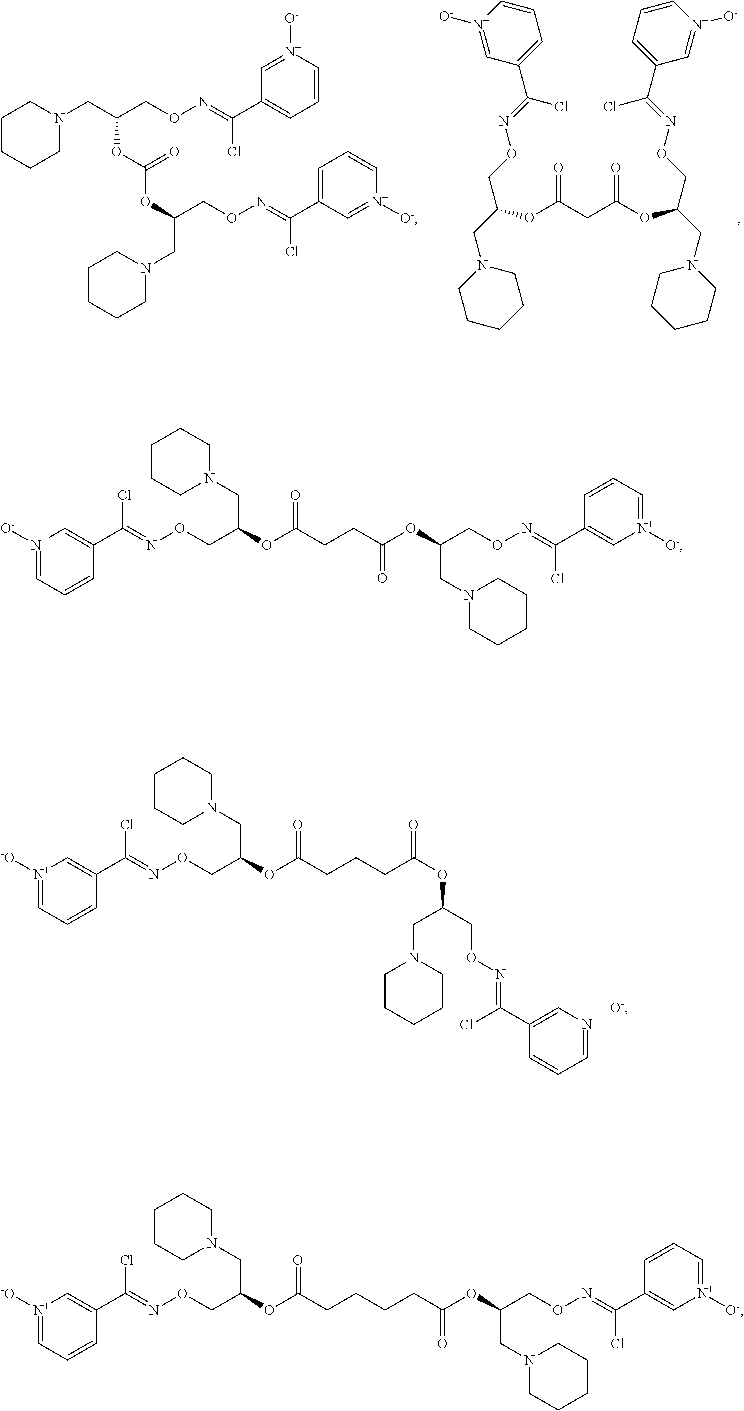 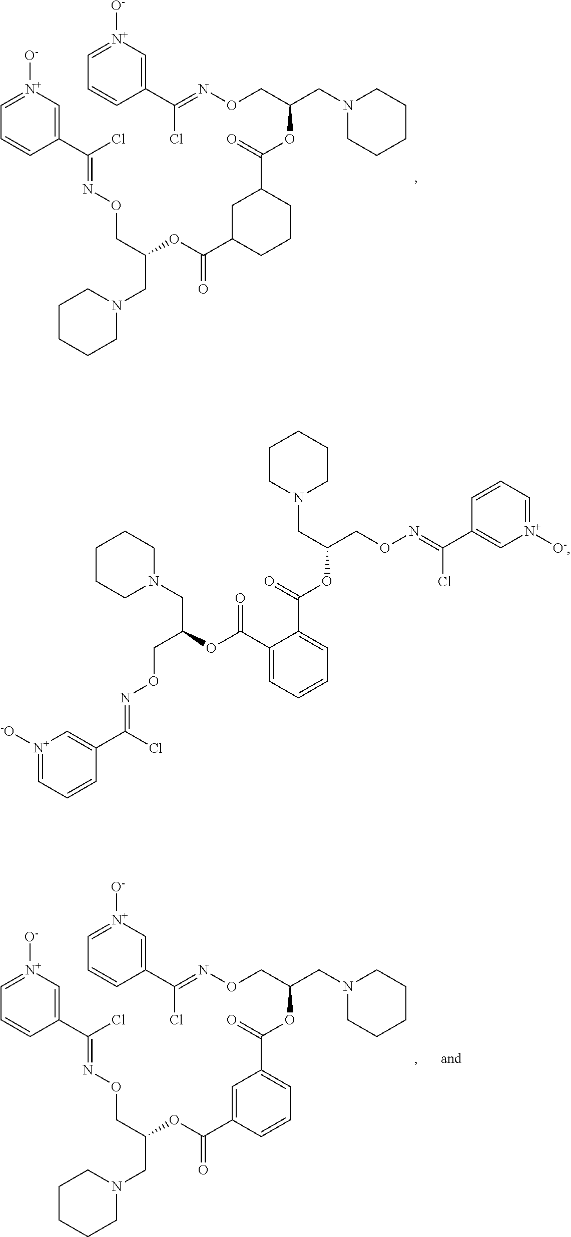 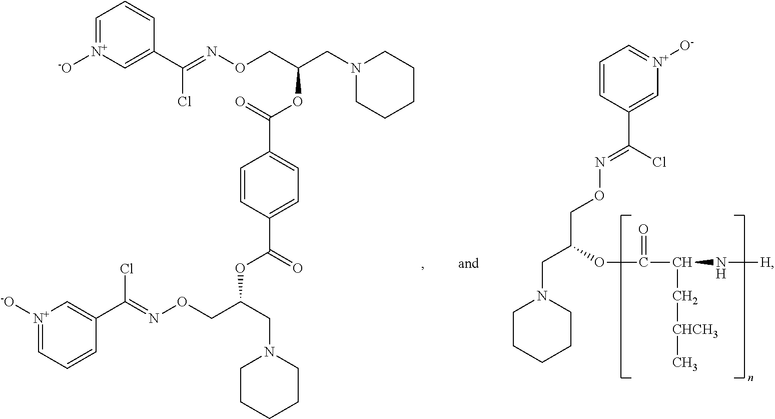
wherein n is 1-50;
or a pharmaceutically acceptable salt thereof.

157. The compound of claim 149, wherein the compound of Formula I is a compound of Formula ID: 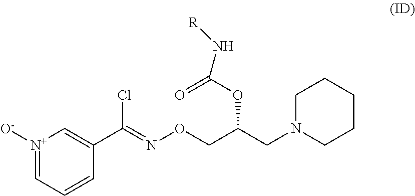
wherein R is selected from the group consisting of alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

158. The compound of claim 158, wherein the compound of Formula ID has a structure selected from the group consisting of: 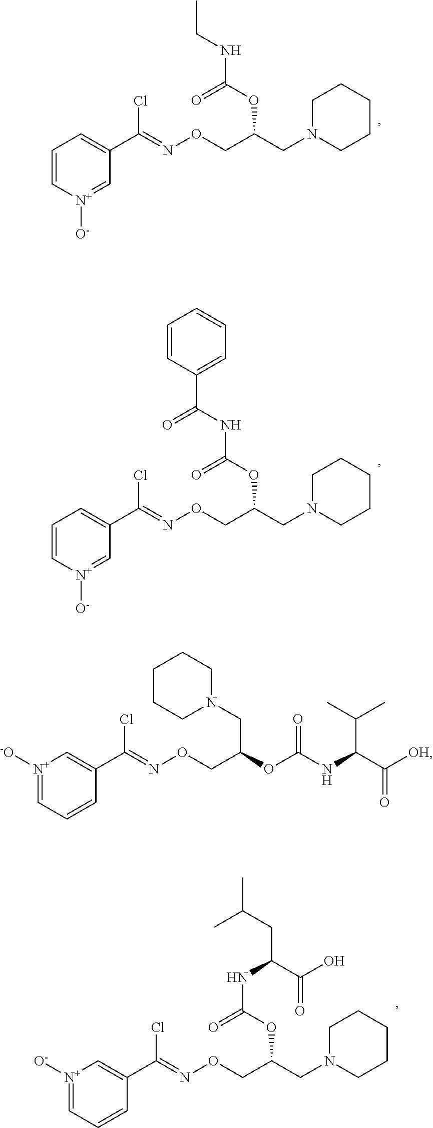 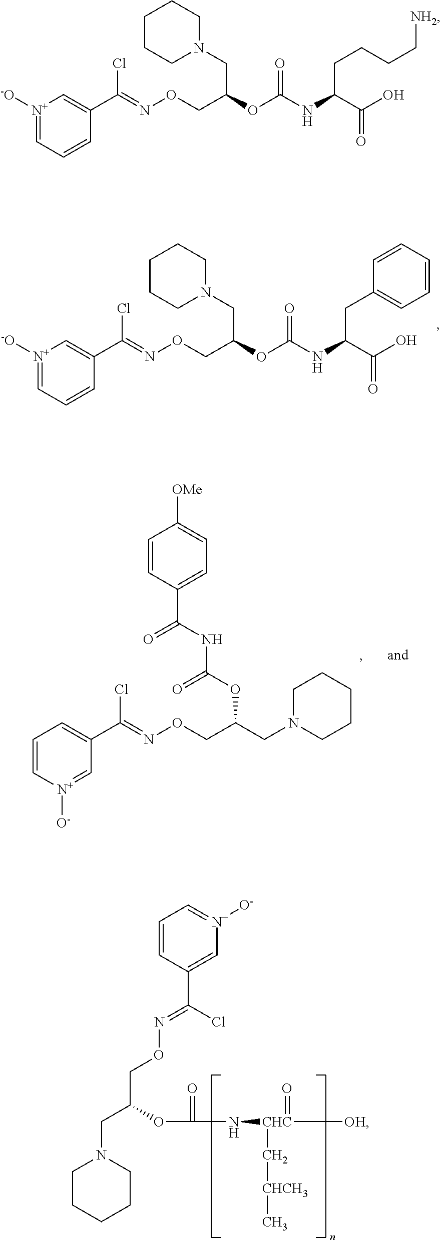
wherein n is 1-50;
or a pharmaceutically acceptable salt thereof.

159. The compound of claim 149, wherein the pharmaceutically acceptable salt is selected from: sulfate, citrate, acetate, oxalate, chloride, hydrochloride, bromide, hydrobromide, iodide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

160. A compound having a structure of Formula II: 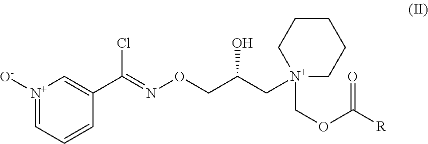
wherein R¹ and R² are independently selected from the group consisting of H, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

161. The compound of claim 160, wherein the compound of Formula II is selected from the group consisting of 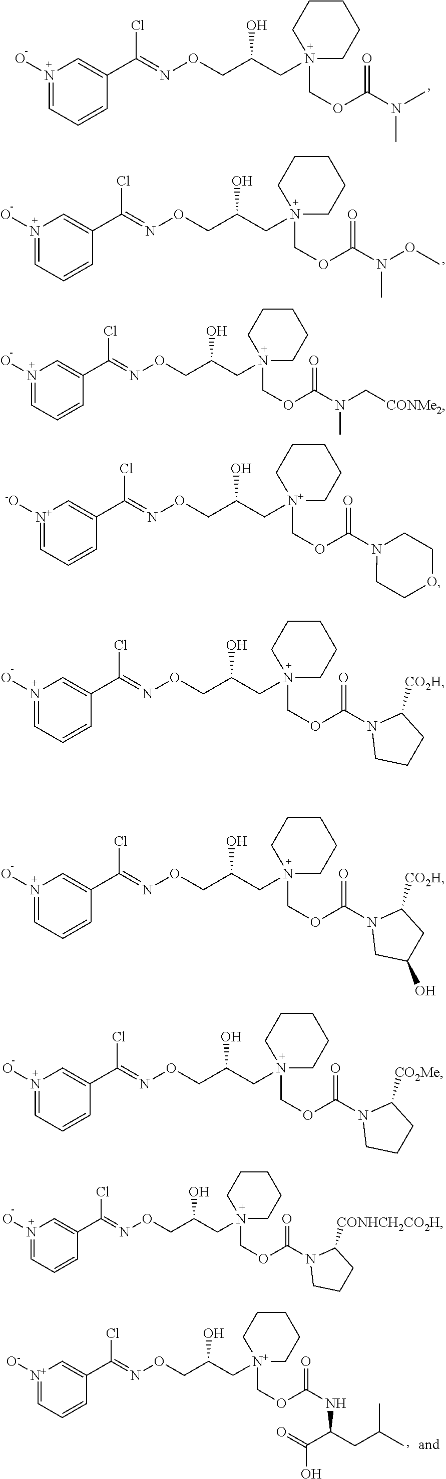 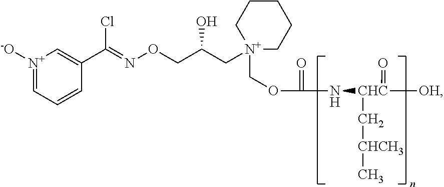
wherein n is 1-50;
or a pharmaceutically acceptable salt thereof.

162. The compound of claim 160, wherein the pharmaceutically acceptable salt is selected from: sulfate, citrate, acetate, oxalate, chloride, hydrochloride, bromide, hydrobromide, iodide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.

163. A compound having a structure of Formula III: 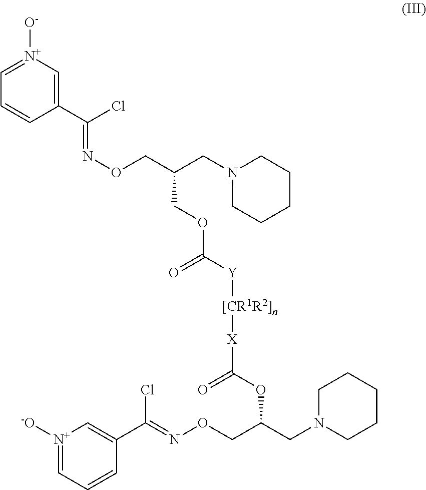
wherein X is selected from the group consisting of O, N, or absent,
Y is selected from the group consisting of O, N, or absent,
n is 1-10,
R¹ and R² are selected independently from each other and for each CR1R2 unit from the group consisting of hydrogen, alkenyl, alkenylaminocarbonyl, alkoxy, alkoxycarbonyl, alkyl, alkylamino, alkylaminocarbonyl, alkylammonium, alkylcarbonyl, alkylcarbonylamino, alkylcarbonyloxy, alkylsulfinyl, alkylsulfonyl, alkylthio, alkynyl, alkynylaminocarbonyl, aryl, substituted aryl, arylalkenyl, arylalkoxy, arylalkyl, arylalkynyl, arylamino, arylaminocarbonyl, arylammonium, arylazo, arylcarbonyl, arylcarbonylamino, arylcarbonyloxy, arylcycloalkyl, aryloxy, aryloxyalkyl, arylsulfinyl, arylsulfinylalkyl, arylsulfonyl, arylsulfonylamino, arylthio, arylthioalkyl, cycloalkenyl, cycloalkenylalkyl, cycloalkyl, cycloalkylalkyl, cycloalkylamino, cycloalkyloxy, cycloalkynyl, cycloheteroalkyl, cycloheteroalkylalkyl, haloalkoxy, haloalkyl, heteroaryl, heteroarylalkenyl, heteroarylalkyl, heteroarylamino, heteroarylcarbonyl, heteroarylcarbonylamino, heteroaryloxo, heteroaryloxy, heteroarylsulfinyl, heteroarylsulfonyl, heteroarylthio, polycycloalkenyl, polycycloalkenylalkyl, polycycloalkyl, polycycloalkylalkyl, and polyethylene glycol;
or a pharmaceutically acceptable salt thereof.

164. The compound of claim 163, wherein the pharmaceutically acceptable salt is selected from: sulfate, citrate, acetate, oxalate, chloride, hydrochloride, bromide, hydrobromide, iodide, hydroiodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisate, fumarate, gluconate, glucuronate, saccharate, formate, benzoate, glutamate, methanesulfonate, ethanesulfonate, benzenesulfonate, p-toluenesulfonate, and pamoate.
